(19) United States
(12) Patent Application Publication
(10) Pub. No.: US 2026/0242478 A1
(43) Pub. Date: Aug. 20, 2026

(54) ANTIBODIES FOR TARGETING PSMA AND CD28 AND USES THEREOF

(71) Applicant: Janux Therapeutics, Inc., San Diego, CA (US)

(72) Inventors: David CAMPBELL, San Diego, CA (US); Thomas R. DIRAIMONDO, San Diego, CA (US)

(21) Appl. No.: 19/659,963

(22) Filed: Apr. 27, 2026

Related U.S. Application Data
(63) Continuation of application No. PCT/US24/54248, filed on Nov. 1, 2024.
(60) Provisional application No. 63/595,921, filed on Nov. 3, 2023.

(51) Int. Cl. C07K 16/28; A61K 45/06; A61P 35/00; C07K 14/705; C07K 16/30

(52) U.S. Cl. CPC C07K 16/2818; A61K 45/06; A61P 35/00; C07K 14/705; C07K 16/3069; C07K 2317/31; C07K 2317/55; C07K 2317/565; C07K 2317/622; C07K 2317/92; C07K 2319/50; C07K 2319/70

(57) ABSTRACT

Disclosed herein are isolated polypeptide and polypeptide complex compositions that comprise a first antigen recognizing molecule that binds to CD28 and a second antigen recognizing molecule that binds to prostate-specific membrane antigen (PSMA).

### CROSS-REFERENCE

The present application is a continuation of International Patent Application No. PCT/US24/54248, filed Nov. 1, 2024, which claims the benefit of U.S. Provisional Application No. 63/595,921 filed on Nov. 3, 2023 which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 25, 2024 is named 52426-764_3011SL.xml and is 4,007,427 bytes in size.

### SUMMARY

Disclosed herein, in some embodiments, are isolated polypeptide or polypeptide complexes that comprise a first antigen recognizing molecule that binds to CD28 and a second antigen recognizing molecule that binds to prostate-specific membrane antigen (PSMA). In some embodiments, the first antigen recognizing molecule that binds to CD28 comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6. In some embodiments, the anti-CD28 heavy chain variable domain comprises an amino acid sequence with at least 80%, 85%, 90%, 95%, 98%, 99%, or 100% sequence identity to SEQ ID NO: 7, and the anti-CD28 light chain variable domain comprises an amino acid sequence with at least 80%, 85%, 90%, 95%, 98%, 99%, or 100% sequence identity to SEQ ID NO: 8.

In some embodiments, the anti-CD28 antibody comprises a single chain variable fragment (scFv), a single domain antibody, a Fab, or a Fab′. In some embodiments, the anti-CD28 antibody comprises the scFv. In some embodiments, the anti-CD28 antibody comprises a single chain variable fragment (scFv) with an engineered disulfide. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 9. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 10. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 11. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 12.

In some embodiments, the anti-CD28 antibody comprises the Fab. In some embodiments, the Fab comprises an amino acid sequence with at least 80%, 85%, 90%, 95%, 98%, 99%, or 100% sequence identity to SEQ ID NO: 696 and an amino acid sequence with at least 80%, 85%, 90%, 95%, 98%, 99%, or 100% sequence identity to SEQ ID NO: 697.

In some embodiments, the anti-CD28 antibody comprises the CrossFab. In some embodiments, the CrossFab comprises an amino acid sequence with at least 80%, 85%, 900, 95%, 98%, 99%, or 100% sequence identity to SEQ ID NO: 698 and an amino acid sequence with at least 80%, 85%, 90%, 95%, 98%, 99%, or 100% sequence identity to SEQ ID NO: 699. In some embodiments, the CrossFab comprises an amino acid sequence with at least 80%, 85%, 90%, 95%, 98%, 99%, or 100% sequence identity to SEQ ID NO: 700 and an amino acid sequence with at least 80%, 85%, 90%, 95%, 98%, 99%, or 100% sequence identity to SEQ ID NO: 701.

In some embodiments, the second antigen recognizing molecule that binds to PSMA comprises an anti-PSMA antibody. In some embodiments, the anti-PSMA antibody comprises an anti-PSMA heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDRL: SEQ ID NO: 13, HC-CDR2: SEQ ID NO: 14, and HC-CDR3: SEQ ID NO: 15 and the anti-PSMA antibody comprises an anti-PSMA light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprise amino acid sequences of LC-CDR1: SEQ ID NO: 16, LC-CDR2: (EA), and LC-CDR3: SEQ ID NO: 18. In some embodiments, the anti-PSMA heavy chain variable domain comprises an amino acid sequence with at least 80%, 85%, 90%, 95%, 98%, 99%, or 100% sequence identity to SEQ ID NO: 20, and the anti-PSMA light chain variable domain comprises an amino acid sequence with at least 80%, 85%, 90%, 95%, 98%, 99%, or 100% sequence identity to SEQ ID NO: 19. In some embodiments, the anti-PSMA antibody comprises a single chain variable fragment (scFv), a single domain antibody, a Fab, a Fab′, or a CrossFab. In some embodiments, the anti-PSMA antibody comprises the Fab or Fab′. In some embodiments, the anti-PSMA antibody comprises the Fab or Fab′, and the Fab or Fab′ comprises an amino acid sequence with at least 80%, 85%, 90°%, 95%, 98%, 99%, or 100% sequence identity to SEQ ID NO: 19 and an amino acid sequence with at least at least 80%, 85%, 90%, 95%, 98%, 99%, or 100% sequence identity to SEQ ID NO: 20. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 702 and an amino acid sequence with at least at least 80%, 85%, 90%, 95%, 98%, 99%, or 100% sequence identity to SEQ ID NO: 703. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 704 and an amino acid sequence with at least at least 80%, 85%, 90%, 95%, 98%, 99%, or 100% sequence identity to SEQ ID NO: 705.

In some embodiments, the anti-CD28 antibody comprises a scFv and the anti-PSMA antibody comprises a Fab or Fab′. In some embodiments, the scFv and the Fab or Fab′ are connected through a linker and the Fab or Fab′ comprise a Fab heavy chain polypeptide and a Fab light chain polypeptide. In some embodiments, the linker connects the scFv to the Fab heavy chain polypeptide. In some embodiments, the linker connects the scFv to the Fab light chain polypeptide. In some embodiments, the linker connects the scFv to the N-term of the Fab heavy chain polypeptide. In some embodiments, the linker connects the scFv to the C-term of the Fab heavy chain polypeptide. In some embodiments, the linker connects the scFv to the N-term of the Fab light chain polypeptide. In some embodiments, the linker connects the scFv to the C-term of the Fab light heavy chain polypeptide. In some embodiments, the linker connects the anti-CD28 heavy chain variable domain of the scFv to the Fab heavy chain polypeptide. In some embodiments, the linker connects the anti-CD28 heavy chain variable domain of the scFv to the Fab light chain polypeptide. In some embodiments, the linker connects the anti-CD28 heavy chain variable domain of the scFv to the N-term of the Fab heavy chain polypeptide. In some embodiments, the linker connects the anti-CD28 heavy chain variable domain of the scFv to the C-term of the Fab heavy chain polypeptide. In some embodiments, the linker connects the anti-CD28 heavy chain variable domain of the scFv to the N-term of the Fab light chain polypeptide. In some embodiments, the linker connects the anti-CD28 heavy chain variable domain of the scFv to the C-term of the Fab light chain polypeptide. In some embodiments, the linker connects the anti-CD28 light chain variable domain of the scFv to the Fab heavy chain polypeptide. In some embodiments, the linker connects the anti-CD28 light chain variable domain of the scFv to the Fab light chain polypeptide. In some embodiments, the linker connects the anti-CD28 light chain variable domain of the scFv to the N-term of the Fab heavy chain polypeptide. In some embodiments, the linker connects the anti-CD28 light chain variable domain of the scFv to the C-term of the Fab heavy chain polypeptide. In some embodiments, the linker connects the anti-CD28 light chain variable domain of the scFv to the N-term of the Fab light chain polypeptide. In some embodiments, the linker connects the anti-CD28 light chain variable domain of the scFv to the C-term of the Fab light chain polypeptide.

In some embodiments, the anti-CD28 antibody comprises a Fab or Fab′ and the anti-PSMA antibody comprises a Fab or Fab′. In some embodiments, the Fab or Fab′ of the anti-CD28 antibody and the Fab or Fab′ of the anti-PSMA antibody are connected through a linker and the Fab or Fab′ of the anti-PSMA antibody comprise a Fab heavy chain polypeptide and a Fab light chain polypeptide. In some embodiments, the linker connects the Fab or Fab′ of the anti-CD28 antibody to the Fab heavy chain polypeptide of the anti-PSMA antibody. In some embodiments, the linker connects the Fab or Fab′ of the anti-CD28 antibody to the Fab light chain polypeptide of the anti-PSMA antibody. In some embodiments, the linker connects the Fab or Fab′ of the anti-CD28 antibody to the N-term of the Fab heavy chain polypeptide of the anti-PSMA antibody. In some embodiments, the linker connects the Fab or Fab′ of the anti-CD28 antibody to the C-term of the Fab heavy chain polypeptide of the anti-PSMA antibody. In some embodiments, the linker connects the Fab or Fab′ of the anti-CD28 antibody to the N-term of the Fab light chain polypeptide of the anti-PSMA antibody. In some embodiments, the linker connects the Fab or Fab′ of the anti-CD28 antibody to the C-term of the Fab light chain polypeptide of the anti-PSMA antibody. In some embodiments, the linker connects a Fab heavy chain polypeptide of the anti-CD28 antibody to the Fab heavy chain polypeptide of the anti-PSMA antibody. In some embodiments, the linker connects a Fab heavy chain polypeptide of the anti-CD28 antibody to the Fab light chain polypeptide of the anti-PSMA antibody. In some embodiments, the linker connects a Fab heavy chain polypeptide of the anti-CD28 antibody to the N-term of the Fab heavy chain polypeptide of the anti-PSMA antibody. In some embodiments, the linker connects a Fab heavy chain polypeptide of the anti-CD28 antibody to the C-term of the Fab heavy chain polypeptide of the anti-PSMA antibody. In some embodiments, the linker connects a Fab heavy chain polypeptide of the anti-CD28 antibody to the N-term of the Fab light chain polypeptide of the anti-PSMA antibody. In some embodiments, the linker connects a Fab heavy chain polypeptide of the anti-CD28 antibody to the C-term of the Fab light chain polypeptide of the anti-PSMA antibody. In some embodiments, the linker connects a Fab light chain polypeptide of the anti-CD28 antibody to the Fab heavy chain polypeptide of the anti-PSMA antibody. In some embodiments, the linker connects a Fab light chain polypeptide of the anti-CD28 antibody to the Fab light chain polypeptide of the anti-PSMA antibody. In some embodiments, the linker connects a Fab light chain polypeptide of the anti-CD28 antibody to the N-term of the Fab heavy chain polypeptide of the anti-PSMA antibody. In some embodiments, the linker connects a Fab light chain polypeptide of the anti-CD28 antibody to the N-term of the Fab light chain polypeptide of the anti-PSMA antibody. In some embodiments, the linker connects a Fab light chain polypeptide of the anti-CD28 antibody to the C-term of the Fab light chain polypeptide of the anti-PSMA antibody.

In some embodiments, the anti-CD28 antibody comprises a CrossFab and the anti-PSMA antibody comprises a Fab or Fab′. In some embodiments, the CrossFab and the Fab or Fab′ are connected through a linker and the Fab or Fab′ of the anti-PSMA antibody comprise a Fab heavy chain polypeptide and a Fab light chain polypeptide. In some embodiments, the linker connects the CrossFab to the Fab heavy chain polypeptide of the anti-PSMA antibody. In some embodiments, the linker connects the CrossFab to the Fab light chain polypeptide of the anti-PSMA antibody. In some embodiments, the linker connects the CrossFab to the N-term of the Fab heavy chain polypeptide of the anti-PSMA antibody. In some embodiments, the linker connects the CrossFab to the C-term of the Fab heavy chain polypeptide of the anti-PSMA antibody. In some embodiments, the linker connects the CrossFab to the N-term of the Fab light chain polypeptide of the anti-PSMA antibody. In some embodiments, the linker connects the CrossFab to the C-term of the Fab light chain polypeptide of the anti-PSMA antibody. In some embodiments, the linker connects a Fab heavy chain polypeptide of the CrossFab to the Fab heavy chain polypeptide of the anti-PSMA antibody. In some embodiments, the linker connects a Fab heavy chain polypeptide of the CrossFab to the Fab light chain polypeptide of the anti-PSMA antibody. In some embodiments, the linker connects a Fab heavy chain polypeptide of the CrossFab to the N-term of the Fab heavy chain polypeptide of the anti-PSMA antibody. In some embodiments, the linker connects a Fab heavy chain polypeptide of the CrossFab to the C-term of the Fab heavy chain polypeptide of the anti-PSMA antibody. In some embodiments, the linker connects a Fab heavy chain polypeptide of the Cross-Fab to the N-term of the Fab light chain polypeptide of the anti-PSMA antibody. In some embodiments, the linker connects a Fab heavy chain polypeptide of the CrossFab to the C-term of the Fab light chain polypeptide of the anti-PSMA antibody. In some embodiments, the linker connects a Fab light chain polypeptide of the CrossFab to the Fab heavy chain polypeptide of the anti-PSMA antibody. In some embodiments, the linker connects a Fab light chain polypeptide of the CrossFab to the Fab light chain polypeptide of the anti-PSMA antibody. In some embodiments, the linker connects a Fab light chain polypeptide of the CrossFab to the N-term of the Fab heavy chain polypeptide of the anti-PSMA antibody. In some embodiments, the linker connects a Fab light chain polypeptide of the CrossFab to the C-term of the Fab heavy chain polypeptide of the anti-PSMA antibody. In some embodiments, the linker connects a Fab light chain polypeptide of the CrossFab to the N-term of the Fab light chain polypeptide of the anti-PSMA antibody. In some embodiments, the linker connects a Fab light chain polypeptide of the CrossFab to the C-term of the Fab light chain polypeptide of the anti-PSMA antibody.

In some embodiments, the anti-CD28 antibody comprises a Fab or Fab′ and the anti-PSMA antibody comprises a CrossFab.

In some embodiments, the linker is at least 5 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker is 15 amino acids in length. In some embodiments, the linker comprises an amino acid sequence of SEQ ID NO: 21 (GGGGSGGGGSGGGGS) or SEQ ID NO: 22 (GGGGS).

In some embodiments, the isolated polypeptide or polypeptide complex comprises amino acid sequences with at least 80%, 85%, 90%, 95%, 98%, 99%, or 100% sequence identity to SEQ ID NO: 23 and SEQ ID NO: 24. In some embodiments, the isolated polypeptide or polypeptide complex comprises amino acid sequences with at least 80%, 85%, 90%, 95%, 98%, 99%, or 100% sequence identity to SEQ ID NO: 25 and SEQ ID NO: 26. In some embodiments, the isolated polypeptide or polypeptide complex comprises amino acid sequences with at least 80%, 85%, 90%, 95%, 98%, 99%, or 100% sequence identity to SEQ ID NO: 27 and SEQ ID NO: 28. In some embodiments, the isolated polypeptide or polypeptide complex comprises amino acid sequences with at least 80%, 85%, 90%, 95%, 98%, 99%, or 100% sequence identity to SEQ ID NO: 29 and SEQ ID NO: 30. In some embodiments, the isolated polypeptide or polypeptide complex comprises amino acid sequences with at least 80%, 85%, 90%, 95%, 98%, 99%, or 100% sequence identity to SEQ ID NO: 31 and SEQ ID NO: 32. In some embodiments, the isolated polypeptide or polypeptide complex comprises the amino acid sequences of SEQ ID NO: 25 and SEQ ID NO: 26. In some embodiments, the isolated polypeptide or polypeptide complex comprises amino acid sequences with at least 80%, 85%, 90%, 95%, 98%, 99%, or 100% sequence identity to SEQ ID NO: 706, and SEQ ID NO: 707. In some embodiments, the isolated polypeptide or polypeptide complex comprises amino acid sequences with at least 80%, 85%, 90%, 95%, 98%, 99%, or 100% sequence identity to SEQ ID NO: 708, SEQ ID NO: 709, and SEQ ID NO: 710. In some embodiments, the isolated polypeptide or polypeptide complex comprises amino acid sequences with at least 80%, 85%, 90%, 95/a, 98%, 99%, or 100% sequence identity to SEQ ID NO: 711, SEQ ID NO: 712, and SEQ ID NO: 713. In some embodiments, the isolated polypeptide or polypeptide complex comprises amino acid sequences with at least 80%, 85%, 90%, 95%, 98%, 99%, or 100% sequence identity to SEQ ID NO: 714, SEQ ID NO: 715, and SEQ ID NO: 716. In some embodiments, the isolated polypeptide or polypeptide complex comprises amino acid sequences with at least 80%, 85%, 90%, 95%, 98%, 99%, or 100% sequence identity to SEQ ID NO: 717, SEQ ID NO: 718, and SEQ ID NO: 719. In some embodiments, the isolated polypeptide or polypeptide complex comprises amino acid sequences with at least 80%, 85%, 90%, 95%, 98%, 99%, or 100% sequence identity to SEQ ID NO: 720, SEQ ID NO: 721, and SEQ ID NO: 722. In some embodiments, the isolated polypeptide or polypeptide complex comprises amino acid sequences with at least 80%, 85%, 90%, 95%, 98%, 99%, or 100% sequence identity to SEQ ID NO: 723, SEQ ID NO: 724, and SEQ ID NO: 725. In some embodiments, the isolated polypeptide or polypeptide complex comprises amino acid sequences with at least 80%, 85, 90%, 95%, 98/o, 99%, or 100% sequence identity to SEQ ID NO: 726, SEQ ID NO: 727, and SEQ ID NO: 728. In some embodiments, the isolated polypeptide or polypeptide complex comprises amino acid sequences with at least 80%, 85%, 90%, 95%, 98%, 99%, or 100% sequence identity to SEQ ID NO: 729, SEQ ID NO: 730, and SEQ ID NO: 731.

In some embodiments, the isolated polypeptide or polypeptide complex is human or humanized. In some embodiments, the isolated polypeptide or polypeptide complex comprises a peptide that is linked to the anti-CD28 antibody, wherein the peptide impairs binding of the anti-CD28 antibody to CD28. In some embodiments, the isolated polypeptide or polypeptide complex comprises a configuration according to Formula I: A₂A₁-L₁-P₁-H₁, wherein A₂ comprises the anti-PSMA antibody, A₁ comprises the anti-CD28 antibody, L₁ comprises a linking moiety that connects A₁ to P₁ and is a substrate for a tumor specific protease, P₁ comprises the peptide that impairs binding of the anti-CD28 antibody to CD28, and H₁ comprises a half-life extending molecule.

In some embodiments, A₂ further comprises P₂ and L₂, wherein P₂ comprises a peptide that binds to A₂; and L₂ comprises a linking moiety that connects A₂ to P₂ and is a substrate for a tumor specific protease. In some embodiments, the polypeptide or polypeptide complex is according to Formula Ia: P₂-L₂-A₂-A₁-L₁-P₁-H₁. In some embodiments, the anti-CD28 antibody comprises a scFv and the anti-PSMA antibody comprises a Fab or Fab′ and the Fab or Fab′ comprise a Fab heavy chain polypeptide and a Fab light chain polypeptide. In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

In some embodiments, P₁ is bound to A₁ through ionic interactions, electrostatic interactions, hydrophobic interactions, Pi-stacking interactions, or H-bonding interactions, or a combination thereof. In some embodiments, P₁ has less than 70% sequence homology to CD28. In some embodiments, P₂ impairs binding of A₂ to PSMA. In some embodiments, P₂ is bound to A₂ through ionic interactions, electrostatic interactions, hydrophobic interactions, Pi-stacking interactions, or H-bonding interactions, or a combination thereof. In some embodiments, P₂ is bound to A₂ at or near an antigen binding site. In some embodiments, P₂ has less than 70% sequence homology to PSMA. In some embodiments, P₁ or P₂ comprises a peptide sequence of at least 10 amino acids in length. In some embodiments, P₁ or P₂ comprises a peptide sequence of at least 10 amino acids in length and no more than 20 amino acids in length. In some embodiments, P₁ or P₂ comprises a peptide sequence of at least 16 amino acids in length. In some embodiments, P₁ or P₂ comprises a peptide sequence of no more than 40 amino acids in length. In some embodiments, P₁ or P₂ comprises at least two cysteine amino acid residues. In some embodiments. P₁ or P₂ comprises a cyclic peptide or a linear peptide. In some embodiments, P₁ or P₂ comprises a cyclic peptide or a linear peptide. In some embodiments, P₁ or P₂ comprises a cyclic peptide. In some embodiments, P₁ or P₂ comprises a linear peptide. In some embodiments, P₁ comprises at least two cysteine amino acid residues.

In some embodiments, P₁ comprises an amino acid sequence according to any one of SEQ ID NOs: 33 to 106, or 732-813. In some embodiments, P₁ comprises the amino acid sequence of SEQ ID NO: 73 or a peptide sequence that has 1, 2, or 3 amino acid substitutions, additions, or deletions relative to the amino acid sequence of SEQ ID NO: 73. In some embodiments, P₁ comprises the amino acid sequence of SEQ ID NO: 73. In some embodiments, P₁ comprises the amino acid sequence of SEQ ID NO: 103 or a peptide sequence that has 1, 2, or 3 amino acid substitutions, additions, or deletions relative to the amino acid sequence of SEQ ID NO: 103. In some embodiments, P₁ comprises the amino acid sequence of SEQ ID NO: 103. In some embodiments, P₁ comprises an amino acid sequence according to X₁-X₂-X₃-C-X₄-X₅-X₆ X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₁ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L. In some embodiments, X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₁ is selected from L, M, and R; X₈ is W; X₉ is selected from H, N, D, A, S, and V; X₁₀ is selected from E, V, L, D, and H; X₁₁ is selected from F, Y, and L; and X₁₂ is selected from N, A, F, S, and Y. In some embodiments, X₁ is M; X₂ is selected from D and H; X₃ is W; X₄ is P; X₅ is selected from R, T, and I; X₆ is selected from E, D, and Y; X₁ is selected from L, M, and R; X₈ is W; X₉ is selected from H, N, D, and V; X₁₀ is selected from E, V, L, D, and H: X₁₁ is F; and Xn₂ is selected from N, A, and F. In some embodiments, P₁ comprises an amino acid sequence according to SEQ ID NO: 41 or an amino acid sequence that has 1, 2, or 3 amino acid mutations, substitutions, or deletions relative to SEQ ID NO: 41. In some embodiments, P₁ comprises an amino acid sequence according to SEQ ID NO: 41. In some embodiments, P₁ comprises an amino acid sequence selected from any one of SEQ ID NOs: 41, 73, 103, and 179-603.

In some embodiments, P₁ comprises an amino acid sequence according to: J₁-J₂-W-C-J₃-J₄-J₅-J₆-J₇-J₈-J₉-C-J₁₀-J₁₁, wherein J₁ is selected from A, R, N, D, Q, E, G, H, I, L, K, F, P, S, T, W, Y, and V; J₂ is selected from D, P, and Y; J₃ is selected from P and Q; J₄ is selected from R, L, and I; J₄ is selected from R. L, and I: J₅ is selected from D, H, Y, and I; J₅ is selected from D, H, Y, and I; J₆ is selected from L, S, and R; J₇ is selected from W and G: J₅ is selected from V, A, D, H, L, and N; J₉ is selected from H, L, E, and D: J₁₀ is selected from F, Y, and L; and J₁₁ is selected from A, N, H, and T. In some embodiments, J₁ is selected from A, R, N, D, Q, E, G, H, K, F, P, S, T, W, and Y. In some embodiments, J₁ is selected from R, Q, E, H I, L, K, F, T, W, Y, and V; J₂ is D; J₃ is P: J is P: J₄ is R; J₅ is D; J₆ is L; J₇ is W; J₈ is V; J₉ is H: J₁₀ is F; and J₁₁ is A. In some embodiments. J₁ is selected from R, Q, E, L, K, T, and W: J₂ is D: J₃ is P: J₄ is R; J₅ is D; J₆ is L; J₇ is W; J₈ is V; J₉ is H, J₁₀ is F; and J₁₁ is A. In some embodiments, J₁ is L or I; J₂ is D or Y; J₃ is P: J₄ is R, L₁ or I; J₅ is H, Y, or D; J₆ is L or R; J₇ is W; J₈ is A, D, H, or N; J₉ is L, E, or D; J₁₀ is F or Y; and J₁₁ is N or H. In some embodiments, J₁ is L or I; J₂ is D or Y: J₃ is P: J₄ is R or I; J₅ is H, Y, or D; J₆ is L or R: J₇ is W; J₈ is A, D, or N; J₉ is L, E, or D; J₁₀ is For Y; and J₁₁ is N.

In some embodiments, P₁ comprises an amino acid sequence according to: Z₁-C-Z₂-Z₃-Z₄-Z₅-Z₆-Z₇-Z₈-Z₉-Z₁₀-Z₁₁-C-Z₁₂, wherein Z, is selected from Y, H, and A; Z₂ is selected from D, E, A, Q, S, V, M, H, L, F, T, W, and N; Z₃ is selected from G, E, S, and A; Z₄ is selected from H, L, W, and A; Z₈ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A: Z₁₀ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A. In some embodiments, Z₁ is selected from Y and H; Z₂ is selected from D, E, A, Q, S, V, M, H, L, F, T, W, and N; Z₃ is selected from G, E, and S; Z₄ is selected from H, L, and W: Z₈ is selected from L, S, A, T, I, V, M, R, E, D, F, H, K, Q, N, Y, W, and G: Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N: Z₈ is selected from Y, H, F, W, N, and Q; Z₉ is selected from M, I, L, R, V, K, F, Q, Y, and W: Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Z₁ is selected from Y and H; Z₂ is selected from D, E, A, Q, S, V, M, H, L, F, T, W, and N: Z₃ is selected from G, E, and S; Z₄ is selected from H, L, and W; Z₈ is selected from L, S, A, T, I, V, M, R, E, D, F, H, K, Q, N, Y, W, and G; Z₆ is selected from P, A, E, Q, S, L, W, G, V, D, K, I, M, R T, F, H, N, and Y; Z₇ is selected from E, Q, I, M, V, A, L, R, W, P, D, T, K, S, G, H, and N; Z₈ is selected from Y, H, F, W, N, and Q; Z₉ is selected from M, I, L, R, V, K, F, Q, Y, and W: Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Z, is Y; Z₂ is selected from D, E, A, and Q; Z₃ is G; Z₄ is H; Z₈ is selected from L, S, A, T, I, V, and M; Z₆ is selected from P, A, E, Q, S, L, W, G, V, and D: Z, is selected from E, Q, I, M, V, A, and L; Z₈ is selected from Y and H; Z₉ is selected from M, I, and L: Z₁₀ is selected from A, Q, S, W, E, L, G, D, T, M, and N: Z₁₁ is K; and Z₁₂ is selected from L and M. In some embodiments, Z₁ is Y; Z₂ is selected from D, E, and A: Z₃ is G; Z₄ is H; Z₄ is H; Z₈ is selected from L, S, A, and T; Z₆ is selected from P, A, E, Q, S, L, and W: Z, is selected from E, Q, I, and M; Z₈ is Y; Z₉ is selected from M, I, and L; Z₁₀ is selected from A. Q, S, W, E, L, G, and D Z₁₁ is K; and Z₁₂ is L. In some embodiments, Z, is Y: Z₂ is selected from D and E; Z₃ is G; Z₄ is H; Z₅ is selected from L, S, and A; Z₆ is selected from P, A, E, Q, S, and L; Z, is selected from E, Q, and I; Z₈ is Y; Z₉ is selected from M, I, and L: Z₁₀ is selected from A, Q, S, W, E, L, G, and D; Z₁₁ is K; and Z₂ is L.

In some embodiments. P₁ comprises the amino acid sequence of SEQ ID NO: 784. In some embodiments, P, consists of the amino acid sequence of SEQ ID NO: 784. In some embodiments, P, comprises the amino acid sequence of SEQ ID NO: 744. In some embodiments, P, consists of the amino acid sequence of SEQ ID NO: 744. In some embodiments, P₁ comprises an amino acid sequence according to any one of SEQ ID NOs: 774-793. In some embodiments, P₁ comprises an amino acid sequence according to any one of SEQ ID NOs: 764-773.

In some embodiments, P₁ comprises an amino acid sequence according to: U₁-C-U₂-U₃-U₄-U₅-U₆-U₇-U₈-U₉-U₁₀-U₁₁-C-U₁₂, wherein: U, is selected from T, S, L, M, W, Q, P, R, G, N, I, Y, D, E, V, H, and A; U₂ is selected from A, S, T, K, E, R, G, N, W, and D; U₃ is selected from A, E, P, D, G, W, Q, V, and F; U₄ is selected from G, V, F, D, E, R, and A; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, I, and A; U₈ is selected from P, S, T, A, E, K, Q, N, V, G, H, L, D, Y, I, M, and R; U₉ is selected from W, L, and A: U₁₀ is selected from P, S, R, L, E, and A; U₁₁ is selected from L, A, T, M, V, W, Y, E, Q, I, F, S, P, K, R, D, H, N, and G; and U₂ is selected from L, F, V, M, I, Y, E, W, and A. In some embodiments, U, is selected from T, S, L, M, W, Q, P, R, G, N, I, Y, D, E, V, and H; U₂ is selected from A, S, T, K, E, R, G, N, W, and D: U₃ is selected from A, E, P, D, G, W, Q, V, and F: U₄ is selected from G, V, F, D, E, and R: U₅ is selected from A, F, V, S, L, P, and M; U₆ is selected from T, S, H, M, A, N, P, G, Q, D, Y, V, L, E, K, F, and R; U₇ is selected from Y, W, F, L, N, T, Q, and I: U₈ is selected from P, S, T, A, E, K, Q, N, V, G, H, L, D, Y, I, M, and R; U₉ is selected from W, L, and A; U₁₀ is selected from P, S, R, L, and E; U₁₁ is selected from L, A, T, M, V, W, Y, E, Q, I, F, S, P, K, R, D, H, N, and G; and U₂ is selected from L, F, V, M, I, Y, E, and W. In some embodiments, U₁ is selected from T, S, L, M, and W; U₂ is selected from A, S, and T; U₃ is selected from A and E; U₄ is selected from G, V, and F; U₈ is selected from A, F, and V; U₆ is selected from T, S, H, M, A, and N; U₇ is selected from Y, W, and F: U₈ is selected from P, S, T, A, E, K, Q, N, and V; U₉ is selected from W and L; U₁₀ is selected from P and S; U₁ is selected from L, A, T, M, V, W, Y, E, Q, I, F, and S; and U₁₂ is selected from L, F, V, and M. In some embodiments, U, is selected from T and S; U₂ is selected from A and S; U₃ is A; U₄ is selected from G and V; U₅ is selected from A and F; U₆ is selected from T, S, and H; U₇ is selected from Y and W; U₈ is selected from P, S, T, and A; U₉ is W; U₁₀ is P; U₁₁ is selected from L, A, T, M, V, W, and Y; and U₁₂ is selected from L and F.

In some embodiments, P₁ comprises the amino acid sequence of SEQ ID NO: 749. In some embodiments, P₁ comprises the amino acid sequence of SEQ ID NO: 749. In some embodiments, P, comprises an amino acid sequence according to any one of SEQ ID NOs: 756-763. In some embodiments, P₁ comprises an amino acid sequence according to any one of SEQ ID NOs: 794-813. In some embodiments, P₁ comprises an amino acid sequence according to any one of SEQ ID NOs: 732-2002.

In some embodiments, P₂ comprises the amino acid sequence of any one of SEQ ID NOs: 2003-4329. In some embodiments, P₂ comprises the amino acid sequence of SEQ ID NO: 2025. In some embodiments, P₂ consists of the amino acid sequence of SEQ ID NO: 2025. In some embodiments. P₂ comprises the amino acid sequence according to any one of SEQ ID NOs: 2087-2094. In some embodiments, P₂ comprises the amino acid sequence of SEQ ID NO: 2026. In some embodiments, P₂ consists of the amino acid sequence of SEQ ID NO: 2026. In some embodiments, P₂ comprises the amino acid sequence according to any one of SEQ ID NOs: 2097-2106. In some embodiments, P₂ comprises the amino acid sequence according to any one of SEQ ID NOs: 2107-2133. In some embodiments. P₂ comprises the amino acid sequence of SEQ ID NO: 2120. In some embodiments. P₂ consists of the amino acid sequence of SEQ ID NO: 2120.

In some embodiments, P₂ comprises an amino acid sequence according to B₁-B₂-B₃-B₄-C-B₅-P-B₆-W-B₇-C-B-B₉-B₁₀(SEQ ID NO: 4470), wherein: B, is selected from V, E, L, D, I, G, M, S, P, T, A, F, W, Y, Q, H, N, K, and R; B₂ is selected from E, V, D, T, S, L, G, P, A, M, I, Q, H, F, Y, N, W, R, and K; B₃ is selected from K, P, R, I, N, H, V, M, A, L, Q, T, S, G, F, Y, E, W, and D; B₄ is selected from W, L, M, R, V, Y, A, K, I, S, Q, F, H, E, T, N, G, and D: B₅ is selected from I, V, T, K, R, E, S, Q M, L, F, A, N, and H; B₆ is selected from A, E, S, P, Q, T, L, D, M, V, R, K, N, I, H, W, Y, F, and G; B₇ is selected from M, L, I, A, V, F, G, and K; B₈ is selected from E, S, T, A, V, D, Q, L, N, I, M, Y, H, F, W, G, K, R, and P; B₉ is selected from G, V, A, S, W, E, D, M, T, L, F, H, Q, N, R, I, Y, P, and K; and B₁₀ is selected from F, L, M, S, I, V, D, Q, T, A, N, Y, W, E, R, H, P, G, and K.

In some embodiments, L₁ is bound to N-terminus of A₁. In some embodiments, L₁ is bound to C-terminus of A₁. In some embodiments, L₂ is bound to N-terminus of A₂. In some embodiments, L₂ is bound to C-terminus of A₂. In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids. In some embodiments, L₁ or L₂ has a formula comprising (G₂S)ₙ, wherein n is an integer from 1 to 3 (SEQ ID NO: 605). In some embodiments, L₁ or L₂ has a formula selected from the group consisting of (G₂S)ₙ, (GS)ₙ, (GSGGS)ₙ (SEQ ID NO: 606), (GGGS)ₙ(SEQ ID NO: 607).(GGGGS).(SEQ ID NO: 608). and (GSSGGS). (SEQ ID NO: 609), wherein n is an integer of at least 1.

In some embodiments, P₁ becomes unbound from A₁ when L₁ is cleaved by the tumor specific protease thereby exposing A₁ to CD28. In some embodiments, P₂ becomes unbound from A₂ when L₂ is cleaved by the tumor specific protease thereby exposing A₂ to PSMA. In some embodiments, the tumor specific protease is selected from the group consisting of a matrix metalloprotease (MMP), serine protease, cysteine protease, threonine protease, and aspartic protease. In some embodiments, the matrix metalloprotease comprises MMP2, MMP7, MMP9, MMP13, or MMP14. In some embodiments, the serine protease comprises matriptase (MTSP1), urokinase, or hepsin. In some embodiments, L₁ or L₂ comprises a urokinase cleavable amino acid sequence, a matriptase cleavable amino acid sequence, matrix metalloprotease cleavable amino acid sequence, or a legumain cleavable amino acid sequence. In some embodiments, L₁ or L₂ comprises an amino acid sequence according to SEQ ID NO: 110. In some embodiments, L₁ or L₂ comprises an amino acid sequence according to any one of SEQ ID NOs: 107-137 and 604. In some embodiments, wherein L₁ or L₂ comprises an amino acid sequence of Linker 25 (ISSGLLSGRSDAG) (SEQ ID NO: 132), Linker 26 (AAGLLAPPGGLSGRSDAG) (SEQ ID NO: 133), Linker 27 (SPLGLSGRSDAG) (SEQ ID NO: 134), or Linker 28 (LSGRSDAGSPLGLAG) (SEQ ID NO: 135), or an amino acid sequence that has 1, 2, or 3 amino acid substitutions, additions, or deletions relative to the amino acid sequence of Linker 25, Linker 26, Linker 27, or Linker 28.

In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, sIgA, IgM or IgD. In some embodiments, the serum protein is albumin. In some embodiments, the polypeptide is an antibody. In some embodiments, the antibody comprises a single domain antibody, a single chain variable fragment, or a Fab. In some embodiments, the single domain antibody comprises a single domain antibody that binds to albumin. In some embodiments, the single domain antibody is a human or humanized antibody. In some embodiments, the single domain antibody is 645gH lgL 1. In some embodiments, the single domain antibody is 645dsgH5gL4. In some embodiments, the single domain antibody is 23-13-A01-sc02. In some embodiments, the single domain antibody is AlOm3 or a fragment thereof. In some embodiments, the single domain antibody is DOM7r-31. In some embodiments, the single domain antibody is DOM7 h-11-15. In some embodiments, the single domain antibody is Alb-1, Alb-8, or Alb-23. In some embodiments, the single domain antibody is 10E. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 142, HC-CDR2: SEQ ID NO: 143, and HC-CDR3: SEQ ID NO: 144. In some embodiments, the single domain antibody comprises an amino acid sequence with at least 80%, 85%, 90%, 95%, 99%, or 100% identity to SEQ ID NO: 141. In some embodiments, the single domain antibody comprises an amino acid sequence with at least 80%, 85%, 90%, 95%, 99%, or 100% identity to SEQ ID NO: 146. In some embodiments, the single domain antibody comprises an amino acid sequence with at least 80%, 85%, 90%, 95%, 99%, or 100% identity to SEQ ID NO: 145. In some embodiments, the single domain antibody comprises an amino acid sequence with at least 80%, 85%, 90%, 95%, 99%, or 100% identity to SEQ ID NO: 4448. In some embodiments, the single domain antibody is SA21. In some embodiments, the polypeptide or polypeptide complex comprises a modified amino acid, a non-natural amino acid, a modified non-natural amino acid, or a combination thereof. In some embodiments, the modified amino acid or modified non-natural amino acid comprises a post-translational modification.

In some embodiments, H₁ comprises a linking moiety (L₃) that connects H₁ to P₁. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS) (SEQ ID NO: 606), (GGGS)ₙ(SEQ ID NO: 607),(GGGGS)ₙ (SEQ ID NO: 608). and (GSSGGS)ₙ (SEQ ID NO: 609), wherein n is an integer of at least 1. In some embodiments, L₃ comprises an amino acid sequence according to GGGGSGGGSGG (SEQ ID NO: 610).

In some embodiments, the polypeptide or polypeptide complex comprises amino acid sequences with at least 95% sequence identity to SEQ ID NOs: 147 and 148. In some embodiments, the polypeptide or polypeptide complex comprises amino acid sequences with at least 95% sequence identity to SEQ ID NOs: 149 and 150. In some embodiments, the polypeptide or polypeptide complex comprises amino acid sequences with at least 95% sequence identity to SEQ ID NOs: 151 and 152. In some embodiments, the polypeptide or polypeptide complex comprises amino acid sequences with at least 95% sequence identity to SEQ ID NOs: 153 and 154. In some embodiments, the polypeptide or polypeptide complex comprises amino acid sequences with at least 95% sequence identity to SEQ ID NOs: 155 and 156. In some embodiments, the polypeptide or polypeptide complex comprises amino acid sequences with at least 95% sequence identity to SEQ ID NOs: 157 and 158. In some embodiments, the polypeptide or polypeptide complex comprises amino acid sequences with at least 95% sequence identity to SEQ ID NOs: 159 and 160. In some embodiments, the polypeptide or polypeptide complex comprises the amino acid sequences of SEQ ID NO: 159 and SEQ ID NO: 160. In some embodiments, the polypeptide or polypeptide complex comprises amino acid sequences with at least 95% sequence identity to SEQ ID NOs: 161 and 162. In some embodiments, the polypeptide or polypeptide complex comprises amino acid sequences with at least 95% sequence identity to SEQ ID NOs: 163 and 164. In some embodiments, the polypeptide or polypeptide complex comprises amino acid sequences with at least 95% sequence identity to SEQ ID NOs: 165 and 166. In some embodiments, the polypeptide or polypeptide complex comprises amino acid sequences with at least 95% sequence identity to SEQ ID NOs: 167 and 168. In some embodiments, the polypeptide or polypeptide complex comprises amino acid sequences with at least 95% sequence identity to SEQ ID NOs: 169 and 170. In some embodiments, the polypeptide or polypeptide complex comprises amino acid sequences with at least 95% sequence identity to SEQ ID NOs: 171 and 172. In some embodiments, the polypeptide or polypeptide complex comprises the amino acid sequences of SEQ ID NO: 171 and SEQ ID NO: 172. In some embodiments, the polypeptide or polypeptide complex comprises amino acid sequences with at least 95% sequence identity to SEQ ID NOs: 173 and 174. In some embodiments, the polypeptide or polypeptide complex comprises amino acid sequences with at least 95% sequence identity to SEQ ID NOs: 175 and 176. In some embodiments, the polypeptide or polypeptide complex comprises amino acid sequences with at least 95% sequence identity to SEQ ID NOs: 177 and 178.

In some embodiments, the polypeptide or polypeptide complex comprises amino acid sequences with at least 80%, 85%, 90%, 95%, 99%, or 100% sequence identity to SEQ ID NO: 4330 and 4331. In some embodiments, the polypeptide or polypeptide complex comprises amino acid sequences with at least 80%, 85%, 90%, 95%, 99%, or 100% sequence identity to SEQ ID NO: 4332 and 4333. In some embodiments, the polypeptide or polypeptide complex comprises amino acid sequences with at least 80%, 85%, 90%, 95%, 99%, or 100% sequence identity to SEQ ID NO: 4334 and 4335. In some embodiments, the polypeptide or polypeptide complex comprises amino acid sequences with at least 80%, 85%, 90%, 95%, 99%, or 100% sequence identity to SEQ ID NO: 4336 and 4337.

In some embodiments, the polypeptide or polypeptide complex comprises amino acid sequences with at least 80%, 85%, 90%, 95%, 99%, or 100% sequence identity to SEQ ID NO: 4338 and 4339. In some embodiments, the polypeptide or polypeptide complex comprises amino acid sequences with at least 80%, 85%, 90%, 95%, 99%, or 100% sequence identity to SEQ ID NO: 4340 and 4341. In some embodiments, the polypeptide or polypeptide complex comprises amino acid sequences with at least 80%, 85%, 90%, 95%, 99%, or 100% sequence identity to SEQ ID NO: 4342 and 4343. In some embodiments, the polypeptide or polypeptide complex comprises amino acid sequences with at least 80%, 85%, 90%, 95%, 99%, or 100% sequence identity to SEQ ID NO: 4344 and 4345. In some embodiments, the polypeptide or polypeptide complex comprises amino acid sequences with at least 80%, 85%, 90%, 95%, 99%, or 100% sequence identity to SEQ ID NO: 4346 and 4347. In some embodiments, the polypeptide or polypeptide complex comprises amino acid sequences with at least 80%, 85%, 90%, 95%, 99%, or 100% sequence identity to SEQ ID NO: 4348 and 4349. In some embodiments, the polypeptide or polypeptide complex comprises amino acid sequences with at least 80%, 85%, 90%, 95%, 99%, or 100% sequence identity to SEQ ID NO: 4350 and 4351. In some embodiments, the polypeptide or polypeptide complex comprises amino acid sequences with at least 80%, 85%, 90%, 95%, 99%, or 100% sequence identity to SEQ ID NO: 4352 and 4353. In some embodiments, the polypeptide or polypeptide complex comprises amino acid sequences with at least 80%, 85%, 90%, 95%, 99%, or 100% sequence identity to SEQ ID NO: 4352 and 4353. In some embodiments, the polypeptide or polypeptide complex comprises amino acid sequences with at least 80%, 85%, 90%, 95%, 99%, or 100% sequence identity to SEQ ID NO: 4354 and 4355. In some embodiments, the polypeptide or polypeptide complex comprises amino acid sequences with at least 80%, 85%, 90%, 95%, 99%, or 100% sequence identity to SEQ ID NO: 4356 and 4357. In some embodiments, the polypeptide or polypeptide complex comprises amino acid sequences with at least 80%, 85%, 90%, 95%, 99%, or 100% sequence identity to SEQ ID NO: 4358 and 4359. In some embodiments, the polypeptide or polypeptide complex comprises amino acid sequences with at least 80%, 85%, 90%, 95%, 99%, or 100% sequence identity to SEQ ID NO: 4360 and 4361. In some embodiments, the polypeptide or polypeptide complex comprises amino acid sequences with at least 80%, 85%, 90%, 95%, 99%, or 100/6 sequence identity to SEQ ID NO: 4362 and 4363. In some embodiments, the polypeptide or polypeptide complex comprises amino acid sequences with at least 80%, 85%, 90%, 95%, 99%, or 100% sequence identity to SEQ ID NO: 4364 and 4365. In some embodiments, the polypeptide or polypeptide complex comprises amino acid sequences with at least 80%, 85%, 90%, 95%, 99%, or 100% sequence identity to SEQ ID NO: 4366 and 4367. In some embodiments, the polypeptide or polypeptide complex comprises amino acid sequences with at least 80%, 85%, 90%, 95%, 99%, or 100% sequence identity to SEQ ID NO: 4368 and 4369. In some embodiments, the polypeptide or polypeptide complex comprises amino acid sequences with at least 80%, 85%, 90%, 95%, 99%, or 100% sequence identity to SEQ ID NO: 4370 and 4371. In some embodiments, the polypeptide or polypeptide complex comprises amino acid sequences with at least 80%, 85%, 90%, 95%, 99%, or 100% sequence identity to SEQ ID NO: 4372 and 4373. In some embodiments, the polypeptide or polypeptide complex comprises amino acid sequences with at least 80%, 85%, 90%, 95%, 99%, or 100% sequence identity to SEQ ID NO: 4374 and 4375. In some embodiments, the polypeptide or polypeptide complex comprises amino acid sequences with at least 80%, 85%, 90%, 95%, 99%, or 100% sequence identity to SEQ ID NO: 4376 and 4377. In some embodiments, the polypeptide or polypeptide complex comprises amino acid sequences with at least 80%, 85%, 90%, 95%, 99%, or 100% sequence identity to SEQ ID NO: 4378 and 4379. In some embodiments, the polypeptide or polypeptide complex comprises amino acid sequences with at least 80%, 85%, 90%, 95%, 99%, or 100% sequence identity to SEQ ID NO: 4380 and 4381. In some embodiments, the polypeptide or polypeptide complex comprises amino acid sequences with at least 80%, 85%, 90%, 95%, 99%, or 100% sequence identity to SEQ ID NO: 4382 and 4383. In some embodiments, the polypeptide or polypeptide complex comprises amino acid sequences with at least 80%, 85%, 90%, 95%, 99%, or 100% sequence identity to SEQ ID NO: 4384 and 4385. In some embodiments, the polypeptide or polypeptide complex comprises amino acid sequences with at least 80%, 85%, 90%, 95%, 99%, or 100% sequence identity to SEQ ID NO: 4386 and 4387.

In some embodiments, the polypeptide or polypeptide complex comprises amino acid sequences with at least 80%, 85%, 90%, 95%, 99%, or 100% sequence identity to SEQ ID NO: 4386 and 4387. In some embodiments, the polypeptide or polypeptide complex comprises amino acid sequences with at least 80%, 85%, 90%, 95%, 99%, or 100% sequence identity to SEQ ID NO: 4388 and 4389. In some embodiments, the polypeptide or polypeptide complex comprises amino acid sequences with at least 80%, 85%, 90%, 95%, 99%, or 100% sequence identity to SEQ ID NO: 4390 and 4391. In some embodiments, the polypeptide or polypeptide complex comprises amino acid sequences with at least 80%, 85%, 90%, 95%, 99%, or 100% sequence identity to SEQ ID NO: 4392 and 4393. In some embodiments, the polypeptide or polypeptide complex comprises amino acid sequences with at least 80%, 85%, 90%, 95%, 99%, or 100% sequence identity to SEQ ID NO: 4394 and 4395. In some embodiments, the polypeptide or polypeptide complex comprises amino acid sequences with at least 80%, 85%, 90%, 95%, 99%, or 100% sequence identity to SEQ ID NO: 4394 and 4395. In some embodiments, the polypeptide or polypeptide complex comprises amino acid sequences with at least 80%, 85%, 90%, 95%, 99%, or 100% sequence identity to SEQ ID NO: 4396 and 4397. In some embodiments, the polypeptide or polypeptide complex comprises amino acid sequences with at least 80%, 85%, 90%, 95%, 99%, or 100% sequence identity to SEQ ID NO: 4398 and 4399. In some embodiments, the polypeptide or polypeptide complex comprises amino acid sequences with at least 80%, 85%, 90%, 95%, 99%, or 100% sequence identity to SEQ ID NO: 4400 and 4401. In some embodiments, the polypeptide or polypeptide complex comprises amino acid sequences with at least 80%, 85%, 90%, 95%, 99%, or 100% sequence identity to SEQ ID NO: 4402 and 4403. In some embodiments, the polypeptide or polypeptide complex comprises amino acid sequences with at least 80%, 85%, 90%, 95%, 99%, or 100% sequence identity to SEQ ID NO: 4404 and 4405. In some embodiments, the polypeptide or polypeptide complex comprises amino acid sequences with at least 80%, 85%, 90%, 95%, 99%, or 100% sequence identity to SEQ ID NO: 4406 and 4407. In some embodiments, the polypeptide or polypeptide complex comprises amino acid sequences with at least 80%, 85%, 90%, 95%, 99%, or 100% sequence identity to SEQ ID NO: 4408 and 4409. In some embodiments, the polypeptide or polypeptide complex comprises amino acid sequences with at least 80%, 85%, 90%, 95%, 99%, or 100% sequence identity to SEQ ID NO: 4410 and 4411. In some embodiments, the polypeptide or polypeptide complex comprises amino acid sequences with at least 80%, 85%, 90%, 95%, 99%, or 100% sequence identity to SEQ ID NO: 4412 and 4413. In some embodiments, the polypeptide or polypeptide complex comprises amino acid sequences with at least 80%, 85%, 90%, 85%, 99%, or 100% sequence identity to SEQ ID NO: 4414 and 4415. In some embodiments, the polypeptide or polypeptide complex comprises amino acid sequences with at least 80%, 85%, 90%, 95%, 99%, or 100% sequence identity to SEQ ID NO: 4416 and 4417. In some embodiments, the polypeptide or polypeptide complex comprises amino acid sequences with at least 80%, 85%, 90%, 95%, 99%, or 100% sequence identity to SEQ ID NO: 4418 and 4419. In some embodiments, the polypeptide or polypeptide complex comprises amino acid sequences with at least 80%, 85%, 90%, 95%, 99%, or 100% sequence identity to SEQ ID NO: 4420 and 4421. In some embodiments, the polypeptide or polypeptide complex comprises amino acid sequences with at least 80%, 85%, 90%, 95%, 99%, or 100% sequence identity to SEQ ID NO: 4422 and 4423. In some embodiments, the polypeptide or polypeptide complex comprises amino acid sequences with at least 80%, 85%, 90%, 95%, 99%, or 100% sequence identity to SEQ ID NO: 4424 and 4425. In some embodiments, the polypeptide or polypeptide complex comprises amino acid sequences with at least 80%, 85%, 90%, 95%, 99%, or 100% sequence identity to SEQ ID NO: 4426 and 4427. In some embodiments, the polypeptide or polypeptide complex comprises amino acid sequences with at least 80%, 85%, 90%, 95%, 99%, or 100% sequence identity to SEQ ID NO: 4428 and 4429. In some embodiments, the polypeptide or polypeptide complex comprises amino acid sequences with at least 80%, 85%, 90%, 95%, 99%, or 100% sequence identity to SEQ ID NO: 4430 and 4431. In some embodiments, the polypeptide or polypeptide complex comprises amino acid sequences with at least 80%, 85%, 90%, 95%, 99%, or 100% sequence identity to SEQ ID NO: 4432 and 4433. In some embodiments, the polypeptide or polypeptide complex comprises amino acid sequences with at least 80%, 85%, 90%, 95%, 99%, or 100% sequence identity to SEQ ID NO: 4434 and 4435. In some embodiments, the polypeptide or polypeptide complex comprises amino acid sequences with at least 80%, 85%, 90%, 95%, 99%, or 100% sequence identity to SEQ ID NO: 4436 and 4437. In some embodiments, the polypeptide or polypeptide complex comprises amino acid sequences with at least 80%, 85%, 90%, 95%, 99%, or 100% sequence identity to SEQ ID NO: 4438 and 4439. In some embodiments, the polypeptide or polypeptide complex comprises amino acid sequences with at least 80%, 85%, 90%, 95%, 99%, or 100% sequence identity to SEQ ID NO: 4440 and 4441. In some embodiments, the polypeptide or polypeptide complex comprises amino acid sequences with at least 80%, 85%, 90%, 95%, 99%, or 100% sequence identity to SEQ ID NO: 4442 and 4443. In some embodiments, the polypeptide or polypeptide complex comprises amino acid sequences with at least 80%, 85%, 90%, 95%, 99%, or 100% sequence identity to SEQ ID NO: 4444 and 4445. In some embodiments, the polypeptide or polypeptide complex comprises amino acid sequences with at least 80%, 85%, 90%, 95%, 99%, or 100% sequence identity to SEQ ID NO: 4446 and 4447.

Disclosed herein, in some embodiments, are isolated polypeptide or polypeptide complexes according to Formula II: L₁ₐ-P₁ₐ-H₁ₐ wherein: L₁ₐ comprises a tumor specific protease-cleaved linking moiety that when uncleaved connects P₁ₐ to an anti-CD28 antibody and the anti-CD28 antibody is connected to an anti-PSMA antibody; P₁ₐ comprises a peptide that impairs binding of the anti-CD28 antibody to CD28, and H₁ₐ comprises a half-life extending molecule. In some embodiments, P₁ₐ when L₁ₐ is uncleaved impairs binding of the anti-CD28 antibody to CD28. In some embodiments, the anti-CD28 antibody comprises a human or humanized antibody.

In some embodiments. H₁ₐ comprises a polymer. In some embodiments, the polymer is polyethylene glycol (PEG). In some embodiments, H₁ₐ comprises albumin. In some embodiments, H₁ₐ comprises an Fc domain. In some embodiments, the albumin is serum albumin. In some embodiments, the albumin is human serum albumin. In some embodiments, H₁ₐ comprises a polypeptide, a ligand, or a small molecule. In some embodiments, the polypeptide, the ligand or the small molecule binds a serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, sIgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the antibody comprises a single domain antibody that binds to albumin. In some embodiments, the antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is AlOm3 or a fragment thereof. In some embodiments, the single domain antibody is DOM7r-31. In some embodiments, the single domain antibody is DOM7 h-11-15. In some embodiments, the single domain antibody is Alb-1, Alb-8, or Alb-23. In some embodiments, the single domain antibody is 10E. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 142, HC-CDR2: SEQ ID NO: 143, and HC-CDR3: SEQ ID NO: 144. In some embodiments, the single domain antibody comprises an amino acid sequence with at least 80%, 85%, 90%, 95%, 99%, or 100% identity to SEQ ID NO: 141. In some embodiments, the single domain antibody comprises an amino acid sequence with at least 80%, 85%, 900, 95%, 99%, or 100% identity to SEQ ID NO: 146. In some embodiments, the single domain antibody comprises an amino acid sequence with at least 80%, 85%, 90%, 95%, 99%, or 100% identity to SEQ ID NO: 145. In some embodiments, the single domain antibody comprises an amino acid sequence with at least 80%, 85%, 90%, 95%, 99%, or 100% identity to SEQ ID NO: 4448. In some embodiments, the single domain antibody is SA21.

In some embodiments, H₁ₐ comprises a linking moiety (L₂ₐ) that connects H₁ₐ to P₁ₐ. In some embodiments, L₂ₐ is a peptide sequence having at least 5 to no more than 50 amino acids. In some embodiments, L₂ₐ is a peptide sequence having at least 10 to no more than 30 amino acids. In some embodiments, L₂ₐ is a peptide sequence having at least 10 amino acids. In some embodiments, L₂ₐ is a peptide sequence having at least 18 amino acids. In some embodiments, L₂ₐ is a peptide sequence having at least 26 amino acids. In some embodiments, L₂ₐ has a formula selected from the group consisting of (G₂S)ₙ, (GS)ₙ, (GSGGS)ₙ (SEQ ID NO: 606), (GGGS),(SEQ ID NO: 607), (GGGGS)ₙ (SEQ ID NO: 608), and (GSSGGS)ₙ(SEQ ID NO: 609), wherein n is an integer of at least 1. In some embodiments, L₂ₐ comprises an amino acid sequence according to SEQ ID NO: 110.

In some embodiments, P₁ₐ has less than 70% sequence homology to CD28. In some embodiments, P₁ₐ comprises a peptide sequence of at least 10 amino acids in length. In some embodiments, P₁ₐ comprises a peptide sequence of at least 10 amino acids in length and no more than 20 amino acids in length. In some embodiments, P₁ₐ comprises a peptide sequence of at least 16 amino acids in length. In some embodiments, P₁ₐ comprises a peptide sequence of no more than 40 amino acids in length. In some embodiments, P₁ₐ comprises at least two cysteine amino acid residues. In some embodiments, Pt, comprises a cyclic peptide or a linear peptide. In some embodiments, P₁ₐ comprises a cyclic peptide. In some embodiments, P₁₈ comprises a linear peptide. In some embodiments, P₁₈ comprises an amino acid sequence according to any one of SEQ ID NOs: 33 to 106, or 732-813. In some embodiments, P₁ₐ comprises the amino acid sequence of SEQ ID NO: 73. In some embodiments, P₁ₐ comprises the amino acid sequence of SEQ ID NO: 784. In some embodiments, P, comprises the amino acid sequence of SEQ ID NO: 73 or a peptide sequence that has 1, 2, or 3 amino acid substitutions, additions, or deletions relative to the amino acid sequence of SEQ ID NO: 73. In some embodiments, P₁ₐ comprises the amino acid sequence of SEQ ID NO: 73. In some embodiments, P₁ₐ comprises the amino acid sequence of SEQ ID NO: 103 or a peptide sequence that has 1, 2, or 3 amino acid substitutions, additions, or deletions relative to the amino acid sequence of SEQ ID NO: 103. In some embodiments, P₁ₐ comprises the amino acid sequence of SEQ ID NO: 103. In some embodiments, P₁ₐ comprises an amino acid sequence according to X₁-X₂-X₃-C-X₄-X₈-X₆-X₇-X₈-X₉-X₁₀-C-X₁₁-X, wherein X₁ is selected from M, I, L, and V; X₂ is selected from D, H, N, A, F, S, T, Y, and V; X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V; X₁ is selected from L, M, R, S, Q, and H; X₈ is selected from W and Q; X is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L. In some embodiments, X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₁ is selected from L, M, and R; X₈ is W; X₉ is selected from H, N, D, A, S, and V; X₁₀ is selected from E, V, L, D, and H; X₁₁ is selected from F, Y, and L; and X is selected from N, A, F, S, and Y. In some embodiments, X₁ is M; X₂ is selected from D and H: X₃ is W: X₄ is P: X₅ is selected from R, T, and I; X₆ is selected from E, D, and Y; X₁ is selected from L, M, and R; X₈ is W; X₉ is selected from H, N, D, and V; X₁₀ is selected from E, V, L, D, and H: X₁₁ is F; and X₁₂ is selected from N, A, and F. In some embodiments, P₁ₐ comprises an amino acid sequence according to SEQ ID NO: 41 or an amino acid sequence that has 1, 2, or 3 amino acid mutations, substitutions, or deletions relative to SEQ ID NO: 41. In some embodiments, P₁ₐ comprises an amino acid sequence according to SEQ ID NO: 41. In some embodiments, P₁ₐ comprises an amino acid sequence selected from any one of SEQ ID NOs: 41, 73, 103, and 179-603.

In some embodiments, P₁, comprises an amino acid sequence according to: J₁-J₂-W-C-J₃-J₄-J₅-J₆-J₇-J₈-J₉-C-J₁₀-J₁₁, wherein J₁ is selected from A, R, N, D, Q, E, G, H, I, L, K, F, P, S, T, W, Y, and V; J₂ is selected from D, P, and Y; J₃ is selected from P and Q; J₄ is selected from R, L, and I; J₄ is selected from R. L, and I: J₅ is selected from D, H, Y, and I; J₅ is selected from D, H, Y, and I; J₆ is selected from L, S, and R; J₇ is selected from W and G: J₅ is selected from V, A, D, H, L, and N; J₉ is selected from H, L, E, and D: J₁₀ is selected from F, Y, and L; and J₁₁ is selected from A, N, H, and T. In some embodiments, J₁ is selected from A, R, N, D, Q, E, G, H, K, F, P, S, T, W, and Y. In some embodiments, J₁ is selected from R, Q, EH, I, L, K, F, T, W, Y, and V; J₂ is D; J₃ is P: J₃ is P: J₄ is R; J₅ is D; J₆ is L; J₇ is W; J₅ is V; J₉ is H: J₁₀ is F; and J₁₁ is A. In some embodiments, J₁ is selected from R, Q, E, L, K, T, and W: J₂ is D: J₃ is P: J₄ is R; J₅ is D; J₆ is L; J₇ is W: J₅ is V; J₉ is H; J₁₀ is F; and J₁₁ is A. In some embodiments, J₁ is L or I; J₂ is D or Y: J₃ is P: J₄ is R, L₁ or I: J₅ is H, Y, or D; J₆ is L or R; J₁₁ is W; J₅ is A, D, H, or N: J₉ is L, E, or D; J₁₀ is F or Y; and J₁₁ is N or H. In some embodiments, J₁ is L or I; J₂ is D or Y: J₃ is P: J₄ is R or I; J₅ is H. Y. or D; J₆ is L or R: J₇ is W; J is A, D, or N; J₉ is L, E, or D; J₁₀ is For Y; and J₁₁ is N.

In some embodiments, P₁ₐ comprises an amino acid sequence according to: Z₁-C-Z₂-Z₃-Z₄-Z₅-Z₁-Z₇-Z₈-Z₉-Z₁₀-Z₁₁-C-Z₁₂, wherein Z, is selected from Y, H, and A; Z₂ is selected from D, E, A, Q, S, V, M, H, L, F, T, W, and N; Z₃ is selected from G, E, S, and A; Z₂, is selected from H, L, W, and A; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A; Z₉ is selected from M, I, L, R, V, K, F, Q, Y, W, and A: Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A. In some embodiments, Z₁ is selected from Y and H; Z₂ is selected from D, E, A, Q, S, V, M, H, L, F, T, W, and N; Z₃ is selected from G, E, and S; Z₄ is selected from H, L, and W; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, and Q: Z₉ is selected from M, I, L, R, V, K, F, Q, Y, and W; Z₁₀ is selected from A, Q, S, W, E, L, G, D, T, M, N, V, H, R, Y, K, P. F, and I; Z₁₁ is selected from K, Q, N, H, E, and P; and Z₁ is selected from L, M, I, F, V, Y, Q, D, and T. In some embodiments, Z₁ is selected from Y and H; Z₂ is selected from D, E, A, Q, S, V, M, H, L, F, T, W, and N; Z₃ is selected from G, E, and S: Z₄ is selected from H, L, and W: Z₈ is selected from L, S, A, T, I, V, M, R, E, D, F, H, K, Q, N, Y, W, and G; Z, is selected from P, A, E, Q, S, L, W, G, V, D, K, I, M, R, T, F, H, N, and Y; Z₇ is selected from E, Q, I, I, V, A, L, R, W, P, D, T, K, S, G, H, and N; Z₈ is selected from Y. H, F, W, N, and Q; Z₉ is selected from M, I, L, R, V, K, F, Q, Y, and W; Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Zi is Y; Z₂ is selected from D, E, A, and Q: Z₃ is G; Z₄ is H; Z₅ is selected from L, S, A, T, I, V, and M: Z₆ is selected from P, A, E, Q, S, L, W, G, V, and D; Z, is selected from E, Q, I, M, V, A, and L; Z₈ is selected from Y and H; Z₉ is selected from M, I, and L; Z₁₀ is selected from A, Q, S, W, E, L, G, D, T, M, and N; Z₁ is K; and Z₁ is selected from L and M. In some embodiments, Z₁ is Y; Z₂ is selected from D, E, and A: Z₃ is G; Z₄ is H; Z₄ is H; Z₈ is selected from L, S, A, and T; Z_{b} is selected from P, A, E, Q, S, L, and W: Z₇ is selected from E, Q, I, and M; Z₈ is Y; Z₉ is selected from M, I, and L; Z₁₀ is selected from A, Q, S, W, E, L, G, and D: Z, is K; and Z₁₂ is L. In some embodiments, Z₁ is Y: Z₂ is selected from D and E; Z₃ is G; Z₄ is H; Z₅ is selected from L, S, and A; Z₆ is selected from P, A, E, Q, S, and L; Z, is selected from E, Q, and I; Z₈ is Y; Z₉ is selected from M, I, and L: Z₁₀ is selected from A, Q, S, W, E, L, G, and D; Z₁₁ is K; and Z₁₂ is L.

In some embodiments, P₁ₐ comprises the amino acid sequence of SEQ ID NO: 784. In some embodiments, P₁ₐ consists of the amino acid sequence of SEQ ID NO: 784. In some embodiments, Pia comprises the amino acid sequence of SEQ ID NO: 744. In some embodiments, P₁ₐ consists of the amino acid sequence of SEQ ID NO: 744. In some embodiments, P₁ₐ comprises an amino acid sequence according to any one of SEQ ID NOs: 774-793. In some embodiments. P₁ₐ comprises an amino acid sequence according to any one of SEQ ID NOs: 764-773.

In some embodiments, P₁ₐ comprises an amino acid sequence according to: U₁-C-U₂-U₃-U₄-U₅-U₆-U₇-U₈-U₉-U₁₀-U₁₁-C-U₁₂, wherein: U, is selected from T, S, L, M, W, Q, P, R, G, N, I, Y, D, E, V, H, and A; U₂ is selected from A, S, T, K, E, R, G, N, W, and D; U₃ is selected from A. E, P, D, G, W, Q, V, and F; U₄ is selected from G, V, F, D, E, R, and A; U₈ is selected from A, F, V, S, L, P, and M: U₆ is selected from T, S. H, M, A. N. P, G, Q, D, Y, V, L, I, E, K, F, and R; U₇ is selected from Y, W, F, L, N, T, Q, I, and A: U₈ is selected from P, S, T, A. E, K, Q, N, V, G, H, L, D, Y, I, M, and R; U₉ is selected from W, L, and A; U₁₀ is selected from P, S, R L, E, and A; U₁₁ is selected from L, A, T, M, V, W, Y, E, Q, I, F, S, P, K, R, D, H, N, and G; and U₁₂ is selected from L, F, V, M, I, Y, E, W, and A. In some embodiments, U₁ is selected from T, S, L, M, W, Q, PR. G, N, Y, D, E, V, and H; U₂ is selected from A. S. T, K, E, R, G, N, W, and D; U₃ is selected from A. E, P, D, G, W, Q, V, and F; U₄ is selected from G, V, F, D, E, and R; U₈ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, and I; U₈ is selected from P, S, T, A, E, K, Q, N, V, G, H, L, D, Y, I, M, and R; U₉ is selected from W, L, and A: U₁₀ is selected from P, S, R, L, and E; U₁₁ is selected from L, A, T, M, V, W, Y, E, Q, I, F, S, P, K, R, D, H, N, and G: and U₁₂ is selected from L, F, V, M, I, Y, E, and W. In some embodiments. U₁ is selected from T, S, L, M, and W. U₂ is selected from A, S, and T; U₃ is selected from A and E: U₄ is selected from G. V, and F; US is selected from A, F, and V; U₆ is selected from T, S, H, M, A, and N; U₇ is selected from Y, W, and F; U₈ is selected from P, S, T, A, E, K, Q, N, and V; U₉ is selected from W and L: U₁₀ is selected from P and S; U₁₁ is selected from L, A, T, M, V, W, Y, E, Q, I, F, and S; and U₁₂ is selected from L, F, V, and M. In some embodiments, U₁ is selected from T and S; U₂ is selected from A and S: U₃ is A; U₄ is selected from G and V; U₈ is selected from A and F; U₆ is selected from T, S, and H: U₇ is selected from Y and W; U₈ is selected from P, S, T, and A: U₉ is W; U₁n is P; U₁₁ is selected from L, A, T, M, V, W, and Y; and U₁₂ is selected from L and F.

In some embodiments. P₁ₐ comprises the amino acid sequence of SEQ ID NO: 749. In some embodiments, P₁ₐ comprises the amino acid sequence of SEQ ID NO: 749. In some embodiments, P₁ₐ comprises an amino acid sequence according to any one of SEQ ID NOs: 756-763. In some embodiments, P₁ₐ comprises an amino acid sequence according to any one of SEQ ID NOs: 794-813. In some embodiments, P₁ₐ comprises an amino acid sequence according to any one of SEQ ID NOs: 732-2002.

Disclosed herein, in some embodiments, are isolated recombinant nucleic acid molecules encoding the polypeptide or polypeptide complex of any one of the embodiments disclosed herein.

Disclosed herein, in some embodiments, are pharmaceutical compositions comprising: (a) the polypeptide or polypeptide complex of any one of the embodiments disclosed herein; and (b) a pharmaceutically acceptable excipient.

Disclosed herein, in some embodiments, are methods of treating cancer in a subject in need thereof comprising administering to the subject the polypeptide or polypeptide complex of any one of the embodiments disclosed herein. In some embodiments, the cancer has cells that express PSMA. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, are methods of treating prostate cancer in a subject need in need thereof comprising administering to the subject an isolated polypeptide or polypeptide complex as described herein. In some embodiments, are methods of treating metastatic castrate-resistant prostate cancer (mCRPC) in a subject need in need thereof comprising administering to the subject an isolated polypeptide or polypeptide complex as described herein.

Disclosed herein, in some embodiments, are methods of treating cancer in a subject in need thereof comprising administering to the subject a polypeptide or polypeptide complex that comprises a first antigen recognizing molecule that binds to CD28 and a second antigen recognizing molecule that binds to prostate-specific membrane antigen. In some embodiments, the first antigen recognizing molecule that binds to CD28 comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6. In some embodiments, the anti-CD28 heavy chain variable domain comprises an amino acid sequence with at least 80%, 85%, 90%, 95%, 98%, 99/a, or 100% sequence identity to SEQ ID NO: 7, and wherein the anti-CD28 light chain variable domain comprises an amino acid sequence with at least 80%, 85%, 90%, 95%, 98%, 99/o, or 100% sequence identity to SEQ ID NO: 8. In some embodiments, the anti-CD28 antibody comprises a single chain variable fragment (scFv), a single domain antibody, a Fab, or a Fab′. In some embodiments, the anti-CD28 antibody comprises the scFv. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 9. In some embodiments, the anti-CD28 antibody comprises the scFv, and the scFv comprises an amino acid sequence with at least 80%, 85%, 90%, 95%, 98/a, 99%, or 100% sequence identity to SEQ ID NO: 10. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 11. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 12.

In some embodiments, the second antigen recognizing molecule that binds to PSMA comprises an anti-PSMA antibody. In some embodiments, the anti-PSMA antibody comprises an anti-PSMA heavy chain variable domain that comprises complementarity determining regions (CDRs): HC-CDR 1, HC-CDR2, and HC-CDR3, wherein the HC-CDR1, the HC-CDR2, and the HC-CDR3 comprise amino acid sequences of HC-CDR1: SEQ ID NO: 13, HC-CDR2: SEQ ID NO: 14, and HC-CDR3: SEQ ID NO: 15 and the anti-PSMA antibody comprises an anti-PSMA light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprise an amino acid sequence of LC-CDR1: SEQ ID NO: 16, LC-CDR2: (EA), and LC-CDR3: SEQ ID NO: 18. In some embodiments, the anti-PSMA heavy chain variable domain comprises an amino acid sequence with at least 80%, 85%, 90%, 95%, 98%, 99%, or 100% sequence identity to SEQ ID NO: 20, and the anti-PSMA light chain variable domain comprises an amino acid sequence with at least 80%, 85%, 90%, 95%, 98%, 99%, or 100% sequence identity to SEQ ID NO: 19. In some embodiments, the anti-PSMA antibody comprises a single chain variable fragment (scFv), a single domain antibody, a Fab, or a Fab′. In some embodiments, the anti-PSMA antibody comprises the Fab or Fab′. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 19 and an amino acid sequence with at least at least 80%, 85%, 90%, 95%, 98%, 99%, or 100% sequence identity to SEQ ID NO: 20.

In some embodiments, the polypeptide or polypeptide complex induces T cell mediated cytotoxicity of tumor cells. In some embodiments, the cancer has cells that express PSMA. In some embodiments, the cancer is a solid tumor cancer. In some embodiments, the cancer is lung, breast, cervical, ovarian, colorectal, pancreatic or gastric cancer.

In some embodiments, the polypeptide or polypeptide complex is administered in combination with an anti-cancer therapy. In some embodiments, the polypeptide or polypeptide complex and the anti-cancer therapy are administered in the same pharmaceutical composition. In some embodiments, the polypeptide or polypeptide complex and the anti-cancer therapy are administered as separate pharmaceutical compositions. In some embodiments, the anti-cancer therapy comprises a small molecule, a cell-based therapy, or an antibody-based therapy.

In some embodiments, the antibody-based therapy is a T cell engager. In some embodiments, the T cell engager comprises a formula according to: D-L₀-E (Formula II), wherein D comprises an effector cell binding domain that binds to an effector cell antigen, E comprises a tumor antigen binding domain that binds to a tumor antigen, and L₀ comprises a linker that connects D to E. In some embodiments, D comprises a single chain variable fragment, a single domain antibody, or a Fab fragment. In some embodiments, D comprises the single chain variable fragment. In some embodiments, E comprises a single chain variable fragment, a single domain antibody, or a Fab fragment. In some embodiments, E comprises the Fab fragment.

In some embodiments, the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, Fl 11-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31. 15865, 15865v12, 15865v16, and 15865v19. In some embodiments, the effector cell binding domain comprises an amino acid sequence according to SEQ ID NOs: 611-624.

In some embodiments, the tumor antigen comprises epidermal growth factor receptor (EGFR), prostate-specific membrane antigen (PSMA), or tumor-associated calcium signal transducer 2 (referred to herein after as TROP2). In some embodiments, the tumor antigen comprises EGFR In some embodiments, the tumor antigen binding domain comprises an amino acid sequence according to SEQ ID NOs: 629-638. In some embodiments, the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 632; HC-CDR2: SEQ ID NO: 633; HC-CDR3: SEQ ID NO: 634; and wherein the LC-CDR1, the LC-CDR2, and the LC-CDR3 comprise: LC-CDR1: SEQ ID NO: 629; LC-CDR2: (YAS); and LC-CDR3: SEQ ID NO: 631. In some embodiments, the tumor antigen comprises EGFR, and the T cell engager comprises amino acid sequences with at least 95% sequence identity according to SEQ ID NOs: 639 and 640. In some embodiments, the tumor antigen comprises EGFR, and the T cell engager comprises amino acid sequences according to SEQ ID NOs: 641 and 642.

In some embodiments, the tumor antigen comprises TROP2. In some embodiments, the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 659; HC-CDR2: SEQ ID NO: 660; HC-CDR3: SEQ ID NO: 661, 662, or 663; and wherein the LC-CDR1, the LC-CDR2, and the LC-CDR3 comprise:LC-CDR 1: SEQ ID NO: 664; LC-CDR2: (SAS); and LC-CDR3: SEQ ID NO: 666. In some embodiments, the tumor antigen comprises TROP2, and the T cell engager comprises amino acid sequences with at least 95% sequence identity according to SEQ ID NOs: 673 and 674. In some embodiments, the tumor antigen comprises TROP2, and the T cell engager comprises amino acid sequences according to SEQ ID NOs: 675 and 676, or SEQ ID NOs: 677 and 678. In some embodiments, the tumor antigen binding domain comprises an amino acid sequence according to SEQ ID NOs: 659-668.

In some embodiments, the tumor antigen comprises PSMA. In some embodiments, the tumor antigen binding domain comprises an amino acid sequence according to SEQ ID NOs: 647-654. In some embodiments, the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 647; HC-CDR2: SEQ ID NO: 648; HC-CDR3: SEQ ID NO: 649; and wherein the LC-CDR1, the LC-CDR2, and the LC-CDR3 comprise:LC-CDR 1: SEQ ID NO: 650; LC-CDR2: SEQ ID NO: 651; and LC-CDR3: SEQ ID NO: 652. In some embodiments, the tumor antigen comprises PSMA, and the T cell engager comprises amino acid sequences with at least 95% sequence identity according to SEQ ID NOs: 655 and 656.

In some embodiments, the T cell engager molecule is selectively activated in tumor microenvironments. In some embodiments, the T cell engager is according to the following subformula: P₃-L₃-D-L₀-E (Formula IIa) wherein D comprises a CD3 binding domain; E comprises the tumor antigen binding domain; L comprises the linker that connects D to E; P₃ comprises a peptide that binds to D and L₃ comprises a linking moiety that connects D to P₃ and is a substrate for a tumor specific protease. In some embodiments, the T cell engager is according to the following subformula: D-L₀-E-Lₐ₁P₄ (Formula IIb) wherein D comprises a CD3 binding domain; E comprises the tumor antigen binding domain; L₀ comprises the linker that connects D to E; P₄ comprises a peptide that binds to E and L₄ comprises a linking moiety that connects E to P₄ and is a substrate for a tumor specific protease. In some embodiments, the T cell engager is according to the following subformula: P₃-L₃-D-L₀-E-Lₐ₁-P₄, (Formula IIc) wherein D comprises a CD3 binding domain; E comprises the tumor antigen binding domain; L₀ comprises the linker that connects D to E: P₃ comprises a peptide that binds to D and L₃ comprises a linking moiety that connects D to P₃ and is a substrate for a tumor specific protease; P₄ comprises a peptide that binds to E and L₄ comprises a linking moiety that connects E to P₄ and is a substrate for a tumor specific protease.

In some embodiments, the T cell engager comprises H₁ In some embodiments, H₁ comprises a sequence according to SEQ ID NO: 141 or SEQ ID NO: 146. In some embodiments, H₁ comprises a single domain antibody. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140.

In some embodiments, L₃ or L₄ is a peptide sequence having at least 5 to no more than 50 amino acids. In some embodiments, L₃ or L₄ is a peptide sequence having at least 10 to no more than 30 amino acids. In some embodiments, L₃ or L₄ is a peptide sequence having at least 10 amino acids. In some embodiments, L₃ or L₄ is a peptide sequence having at least 18 amino acids. In some embodiments. L₃ or L₄ is a peptide sequence having at least 26 amino acids. In some embodiments, L₃ or L₄ has a formula comprising (G₂S)ₙ, wherein n is an integer from 1 to 3 (SEQ ID NO: 605). In some embodiments, L₃ or IU has a formula comprising (G₂S)ₙ, wherein n is an integer of at least 1. In some embodiments, L₃ or L₄ has a formula selected from the group consisting of (G₂S)ₙ, (GS)ₙ, (GSGGS)ₙ (SEQ ID NO: 606), (GGGS)ₙ (SEQ ID NO: 607).(GGGGS)ₙ (SEQ ID NO: 608), and (GSSGGS), (SEQ ID NO: 609), wherein n is an integer of at least 1.

In some embodiments, the tumor specific protease is selected from the group consisting of metalloprotease, serine protease, cysteine protease, threonine protease, and aspartic protease. In some embodiments, L₃ or L₄ comprises a urokinase cleavable amino acid sequence, a matriptase cleavable amino acid sequence, or a matrix metalloprotease cleavable amino acid sequence. In some embodiments, L₃ or L₄ comprises a sequence according to any one of SEQ ID NOs: 21-22 and 107-136. In some embodiments, L₃ is bound to N-terminus of D. In some embodiments, L₃ is bound to C-terminus of D. In some embodiments, IU is bound to N-terminus of E. In some embodiments, L₄ is bound to C-terminus of E.

In some embodiments, P₃ becomes unbound from D when L₃ is cleaved by the tumor specific protease thereby exposing D to CD3. In some embodiments, P₄ becomes unbound from E when L₄ is cleaved by the tumor specific protease thereby exposing E to the tumor antigen. In some embodiments. P₃ impairs binding of D to CD3. In some embodiments, P₃ is bound to D through ionic interactions, electrostatic interactions, hydrophobic interactions, Pi-stacking interactions, and H-bonding interactions, or a combination thereof. In some embodiments, P₃ is bound to D at or near an antigen binding site. In some embodiments, P₃ becomes unbound from D when L₃ is cleaved by the tumor specific protease thereby exposing D to CD3.

In some embodiments, P₃ has less than 70% sequence identity to CD3. In some embodiments, P₃ has less than 70% sequence identity to CD3. In some embodiments, P₃ has less than 85% sequence identity to CD3. In some embodiments, P₃ has less than 90% sequence identity to CD3. In some embodiments, P₃ has less than 95% sequence identity to CD3. In some embodiments, P₃ has less than 98% sequence identity to CD3. In some embodiments, P₃ has less than 99% sequence identity to CD3. In some embodiments, P₃ comprises the amino acid sequence according to SEQ ID NO: 625 or SEQ ID NO: 626. In some embodiments, P₃ comprises a de novo amino acid sequence that shares less than 10% sequence identity to CD3.

In some embodiments, P₄ impairs binding of E to the tumor antigen. In some embodiments, P₄ is bound to E through ionic interactions, electrostatic interactions, hydrophobic interactions, Pi-stacking interactions, and H-bonding interactions, or a combination thereof. In some embodiments, P₄ is bound to E at or near an antigen binding site. In some embodiments, P₄ becomes unbound from E when L₄ is cleaved by the tumor specific protease thereby exposing E to the tumor antigen. In some embodiments, P₄ has less than 70% sequence identity to the tumor antigen. In some embodiments, P₄ has less than 80% sequence identity to the tumor antigen. In some embodiments, P₄ has less than 85% sequence identity to the tumor antigen. In some embodiments, P₄ has less than 90% sequence identity to the tumor antigen. In some embodiments, P₄ has less than 95% sequence identity to the tumor antigen. In some embodiments, P₄ comprises a de novo amino acid sequence that shares less than 10% sequence identity to the tumor antigen.

In some embodiments, P₃ or P₄ comprises a peptide sequence of at least 5 amino acids in length. In some embodiments, P₃ or P₄ comprises a peptide sequence of at least 6 amino acids in length. In some embodiments, P₃ or P₄ comprises a peptide sequence of at least 10 amino acids in length. In some embodiments, P₃ or P₄ comprises a peptide sequence of at least 10 amino acids in length and no more than 20 amino acids in length. In some embodiments, P₃ or P₄ comprises a peptide sequence of at least 16 amino acids in length. In some embodiments, P₃ or P₄ comprises a peptide sequence of no more than 40 amino acids in length. In some embodiments, P₃ or P₄ comprises at least two cysteine amino acid residues. In some embodiments, P₃ or P₄ comprises a cyclic peptide or a linear peptide. In some embodiments, P₃ or P₄ comprises a cyclic peptide. In some embodiments, P₃ or P₄ comprises a linear peptide.

In some embodiments, P₄ comprises the amino acid sequence according to SEQ ID NO: 645. In some embodiments, the tumor antigen comprises EGFR, and the T cell engager comprises the amino acid sequences of SEQ ID NOs: 643 and 644.

In some embodiments, P₄ comprises the amino acid sequence according to any one of SEQ ID NOs: 693-695. In some embodiments, the tumor antigen comprises TROP2, and the T cell engager comprises the amino acid sequences of SEQ ID NOs: 679 and 680, SEQ ID NOs: 681 and 682, SEQ ID NOs: 683 and 684, SEQ ID NOs: 685 and 686, SEQ ID NOs: 687 and 688, SEQ ID NOs: 689 and 690, and SEQ ID NOs: 691 and 692. In some embodiments, the tumor antigen comprises TROP2, and the T cell engager comprises the amino acid sequences of SEQ ID NOs: 4452 and 4453, SEQ ID NOs: 4454 and 4455, SEQ ID NOs: 4456 and 4457, SEQ ID NOs: 4458 and 4459, SEQ ID NOs: 4460 and 4461, SEQ ID NOs: 4462 and 4463, or SEQ ID NOs. 4464 and 4465.

In some embodiments, the tumor antigen comprises PSMA, and the T cell engager comprises the amino acid sequences of SEQ ID NOs: 657 and 658.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a cartoon configuration of a multispecific antibody construct that targets PSMA and CD28 that is administered in combination with a T cell engager (TCE) that targets CD3 and the tumor associated antigen EGFR The density (copies per cell) of PSMA and EGFR on LNCaP tumor cells used in the assay of FIG. 1B is shown in the table.

FIG. 1B shows tumor cell killing of LNCaP tumor cells by EGFR TCE-2 alone, different concentrations of EGFR TCE-2 in combination with Ab-2 (a multispecific antibody targeting PSMA and CD28), and Ab-2 alone as measured using an in vitro hPBMC and tumor cell co-culture assay. EC₅₀ₛ for tumor cell killing are shown in the table.

FIG. 2A illustrates a cartoon configuration of a multispecific antibody construct that targets PSMA and CD28 that is administered in combination with a TCE that targets CD3 and the tumor associated antigen PSMA. The density (copies per cell) of PSMA on LNCaP tumor cells used in the assay of FIG. 2B is shown in the table.

FIG. 2B shows tumor cell killing of LNCaP tumor cells by PSMA TCE-1 alone and different concentrations of PSMA TCE-1 in combination with Ab-2 (a multispecific antibody targeting PSMA and CD28) as measured using an in vitro hPBMC and tumor cell co-culture assay. EC₅₀ₛ for tumor cell killing are shown in the table.

FIG. 3 shows tumor cell killing of LNCaP tumor cells by EGFR TCE-2, EGFR TCE-2 in combination with non-masked Ab-2, EGFR TCE-2 in combination with PC-2 having a peptide mask for CD28, and EGFR TCE-2 in combination with PC-2 after cleavage of the peptide mask with membrane-type serine protease 1 (MTSP-1). EC50s for tumor cell killing are shown in the table.

FIG. 4A illustrates a cartoon configuration of a multispecific antibody construct that targets PSMA and CD28 that is administered in combination with a TCE that targets CD3 and the tumor associated antigen TROP-2. The density (copies per cell) of PSMA and TROP-2 on recombinant PSMA expressing DU-145 cells used in the assays of FIGS. 4B-4C is shown in the table.

FIG. 4B shows IL-2 and IFNγ cytokine production with no stimulation (“no stim”), stimulation with TROP2 TCE-1 (“TROP2xCD3”), and stimulation with a combination of TROP2 TCE-1 and Ab-2 (“TROP2xCD3+PSMAxCD28”) as measured using an in vitro T cell activation assay using PBMCs from a first donor.

FIG. 4C shows IL-2 and IFNγ cytokine production with no stimulation (“no stim”), stimulation with TROP2 TCE-1 (“TROP2xCD3”), and stimulation with a combination of TROP2 TCE-1 and Ab-2 (“TROP2xCD3+PSMAxCD28”) as measured using an in vitro T cell activation assay using PBMCs from a second donor.

FIG. 5A illustrates a cartoon configuration of binding of a multispecific antibody targeting PSMA and CD28 to cytomegalovirus (CMV) peptide-loaded DU-145 cells expressing PSMA and CMV positive PBMCs. The density (copies per cell) of PSMA on the CMV peptide-loaded DU-145 cells used in the assays of FIGS. 5B-5C is shown in the table.

FIG. 5B shows dose-dependent enhancement of IL-2 production with Ab-2 in a tumor cell/PBMC mixed lymphocyte reaction (MLR) system using PBMCs from a first donor.

FIG. 5C shows dose-dependent enhancement of IL-2 production with Ab-2 in a tumor cell/PBMC MLR system using PBMCs from a second donor.

FIG. 6 shows a schematic representation of the tumor-specific activity of a multispecific antibody of the present disclosure having a CD28 binding domain (CD28 BD), a PSMA binding domain (PSMA BD), a peptide mask (CD28 mask) connected to the CD28 binding domain by a cleavable linker, a peptide mask (PSMA mask) connected to the PSMA binding domain by a cleavable linker, and a half-life extending moiety (e.g., an albumin binding domain). In the tumor microenvironment (TME), cleavage of the cleavable linkers by tumor proteases releases the peptide masks and the half-life extending moiety, thereby activating the multispecific antibody for CD28 stimulation in tumor tissue and promoting rapid clearance of the multispecific antibody in healthy tissue.

FIG. 7A shows tumor cell killing of LNCaP tumor cells by EGFR TCE-1 alone, different concentrations of an EGFRTCE-1 in combination with Ab-1, and Ab-1 alone as measured using an in vitro hPBMC and tumor cell co-culture assay.

FIG. 7B shows tumor cell killing of LNCaP tumor cells by EGFR TCE-1 alone, different concentrations of EGFR TCE-1 in combination with Ab-2, and Ab-2 alone as measured using an in vitro hPBMC and tumor cell co-culture assay.

FIG. 8 shows tumor cell killing of LNCaP tumor cells by EGFR TCE-1 alone and EGFR TCE-1 in combination with Ab-2 as measured using an in vitro hPBMC and tumor cell co-culture assay.

FIG. 9 shows tumor cell killing of LNCaP tumor cells by a PSMA TCE-1 alone, PSMA TCE-1 in combination with Ab-2, and PSMA TCE-1 in combination with Ab-4 as measured using an in vitro tumor cell co-culture assay.

FIGS. 10A-10C show tumor cell killing of different cancer cell lines with different PSMA expression with PSMA TCE-1 alone or PSMA TCE-1 in the presence of Ab-2 as measured using an in vitro tumor cell co-culture assay.

FIGS. 11A-11B show tumor cell killing of different cancer cell lines with different PSMA and EGFR expression with EGFR TCE-1 alone or EGFR TCE-1 in the presence of Ab-2 as measured using an in vitro tumor cell co-culture assay.

FIG. 12 shows tumor cell killing of LNCaP cancer cells with PSMA TCE-1 in combination with Ab-4 and PSMA TCE-1 in combination with PC-9 as measured using an in vitro tumor cell co-culture assay.

FIG. 13 shows tumor cell killing of LNCaP cancer cells with PSMA TCE-1 in combination with Ab-4, PSMA TCE-1 in combination with PC-66, and PSMA TCE-1 in combination with PC-66 after MMP9 protease treatment as measured using an in vitro tumor cell co-culture assay.

FIG. 14 shows tumor cell killing of LNCaP cancer cells with a combination of Ab-2 and PSMA TCE-1, and a combination of PC-7 and PSMA TRACTr-1 as measured using an in vitro tumor cell killing assay.

FIG. 15 shows tumor cell killing of LNCaP cancer cells with PSMA TCE-1 alone and a combination of Ab-4 and PSMA TCE-1 as measured using an in vitro tumor cell killing assay.

FIG. 16 shows tumor cell killing of LNCaP cancer cells with combinations of PSMA TCE-1 and Ab-4, PSMA TCE-1 and PC-25, PSMA TCE-1 and PC-33, and PSMA TCE-1 and PC-42.

FIG. 17 shows tumor cell killing of LNCaP cancer cells with combinations PSMA TCE-1 and Ab-2, PSMA TCE-1 and PC-50, PSMA TCE-1 and PC-58, and PSMA TCE-1 and PC-66.

FIG. 18 shows tumor cell killing of LNCaP cancer cells with combinations PSMA TCE-1 and Ab-2, PSMA TCE-1 and PC-25 with MMP9 protease treatment, PSMA TCE-1 and PC-33 with MMP9 protease treatment, and PSMA TCE-1 and PC-42 with MMP9 protease treatment.

FIG. 19 shows tumor cell killing of LNCaP cancer cells with combinations PSMA TCE-1 and Ab-2, PSMA TCE-1 and PC-50 with MMP9 protease treatment, PSMA TCE-1 and PC-58 with MMP9 protease treatment, and PSMA TCE-1 and PC-66 with MMP9 protease treatment.

FIG. 20 shows production of interferon gamma (IFNγ), interleukin-2 (IL-2), and tumor necrosis factor (TNF) following treatment with PSMA TCE-1+Ab-2 and PSMA-TRACTr-1+PC-7 as measured using an in vitro T cell activation assay using PBMCs.

FIG. 21 illustrates PSMA binding by Ab-4, PC-66, and PC-66 with MMP9 treatment as measured by ELISA.

FIG. 22 illustrates CD28 binding by Ab-4, PC-66, and PC-66 with MMP9 treatment as measured by ELISA.

FIG. 23 illustrates binding of Ab-15 to peptides as measured by ELI.

FIG. 24 illustrates binding of Ab-15 to peptides as measured by ELISA.

FIG. 25 illustrates binding of Ab-15 to peptides as measured by ELISA

FIG. 26 illustrates biolayer interferometry (BLI) titration data for Ab-15 binding to peptide-41.

FIG. 27 illustrates BLI titration data for Ab-15 binding to peptide-53.

FIG. 28 illustrates BLI titration data for Ab-15 binding to peptide-56.

FIG. 29 illustrates BLI titration data for Ab-15 binding to peptide-57.

FIG. 30 illustrates BLI titration data for Ab-15 binding to peptide-62.

FIG. 31 illustrates BLI titration data for Ab-15 binding to peptide-66.

FIG. 32 illustrates BLI titration data for Ab-15 binding to peptide-71.

FIG. 33 illustrates BLI titration data for Ab-15 binding to peptide-41.

FIG. 34 illustrates BLI titration data for Ab-15 binding to peptide-288.

FIG. 35 illustrates BLI titration data for Ab-15 binding to peptide-289.

FIG. 36 illustrates inhibition of Ab-15 binding to CD28 by peptides of the present disclosure.

FIG. 37 illustrates inhibition of Ab-15 binding to CD28 by peptides of the present disclosure.

FIG. 38 illustrates inhibition of Ab-15 binding to CD28 by peptides of the present disclosure.

FIG. 39 illustrates binding of anti-CD28 Fab to peptides as measured by ELISA.

FIG. 40 illustrates binding of anti-CD28 Fab to peptides as measured by ELISA.

FIG. 41 illustrates binding of anti-CD28 Fab to peptides as measured by ELISA.

FIG. 42 illustrates binding of anti-CD28 Fab to peptides as measured by ELISA.

FIG. 43 illustrates inhibition of anti-CD28 Fab binding to CD28 by peptides of the present disclosure.

FIG. 44 illustrates inhibition of anti-CD28 Fab binding to CD28 by peptides of the present disclosure.

FIG. 45 illustrates inhibition of anti-CD28 Fab binding to CD28 by peptides of the present disclosure.

FIG. 46 illustrates inhibition of anti-CD28 Fab binding to CD28 by peptides of the present disclosure.

FIG. 47 illustrates inhibition of anti-CD28 scFv disulfide to CD28 by peptides of the present disclosure.

FIG. 48 illustrates BLI titration data for anti-CD28 Fab binding to peptide-87.

FIG. 49 illustrates BLI titration data for anti-CD28 Fab binding to peptide-120.

FIG. 50 illustrates BLI titration data for anti-CD28 Fab binding to peptide-121.

FIG. 51 illustrates BLI titration data for anti-CD28 Fab binding to peptide-126.

FIG. 52 illustrates BLI titration data for anti-CD28 Fab binding to peptide-127.

FIG. 53 illustrates BLI titration data for anti-CD28 Fab binding to peptide-132.

FIG. 54 illustrates BLI titration data for anti-CD28 Fab binding to peptide-134.

FIG. 55 illustrates the core sequence motif of peptide-87 sequences generated using WebLogo 3.7.12.

FIG. 56 illustrates the core sequence motif of anti-CD28 Fab peptide-92 sequences generated using WebLogo 3.7.12.

FIG. 57 illustrates a schematic for identifying peptides that can be attached to anti-CD28 multispecific antibodies for selective activation in tumor microenvironments. The schematic illustrates a directed evolution and phage display technology to identify peptides that block antigen recognition by antigen binding domains.

FIG. 58 illustrates anti-CD28 scFv binding to peptides measured by ELISA.

FIG. 59 illustrates anti-CD28 scFv binding to peptides measured by ELISA.

FIGS. 60-61 illustrate that peptides inhibit anti-CD28 scFv from binding to CD28 antigen as measured by ELISA.

FIGS. 62-63 illustrate kinetic binding of anti-CD28 scFv to pepides as measured by Octet.

FIGS. 64A-64B illustrate binding of anti-CD28 scFv to Ala scan peptides of Peptide-9.

FIGS. 65A-65B illustrate inhibition of anti-CD28 scFv binding to CD28 by Ala scan peptides of Peptide-9.

FIG. 66 illustrates the core sequence motif of optimized anti-CD28 scFv Peptide-9 sequences generated using WebLogo 3.7.4.

FIGS. 67-69 illustrate peptides that inhibit the anti-CD28 scFv from binding the CD28 antigen measured by ELISA.

FIGS. 70A-70U illustrate kinetic binding of anti-CD28 scFv binding to peptides as measured by Octet.

FIGS. 71-72 illustrate binding of PSMA Fab to peptides as measured by ELISA.

FIGS. 73-74 illustrate inhibition of PSMA Fab binding to PSMA by peptides of the present disclosure.

FIG. 75 illustrates BLI titration data for PSMA Fab binding to peptide-179.

FIG. 76 illustrates BLI titration data for PSMA Fab binding to peptide-219.

FIG. 77 illustrates BLI titration data for PSMA Fab binding to peptide-225.

FIG. 78 illustrates BLI titration data for PSMA Fab binding to peptide-227.

FIG. 79 illustrates BLI titration data for PSMA Fab binding to peptide-230.

FIG. 80 illustrates BLI titration data for PSMA Fab binding to peptide-232.

FIG. 81 illustrates BLI titration data for PSMA Fab binding to peptide-233.

FIG. 82 illustrates BLI titration data for PSMA Fab binding to peptide-235.

FIG. 83 illustrates BLI titration data for PSMA Fab binding to peptide-236.

FIG. 84 illustrates BLI titration data for PSMA Fab binding to peptide-239.

FIG. 85 illustrates BLI titration data for PSMA Fab binding to peptide-180.

FIG. 86 illustrates BLI titration data for PSMA Fab binding to peptide-263.

FIG. 87 illustrates BLI titration data for PSMA Fab binding to peptide-267.

FIG. 88 illustrates BLI titration data for PSMA Fab binding to peptide-268.

FIG. 89 illustrates BLI titration data for PSMA Fab binding to peptide-270.

FIG. 90 illustrates BLI titration data for PSMA Fab binding to peptide-271.

FIG. 91 illustrates BLI titration data for PSMA Fab binding to peptide-274.

FIG. 92 illustrates BLI titration data for PSMA Fab binding to peptide-277.

FIG. 93 illustrates BLI titration data for PSMA Fab binding to peptide-282.

FIG. 94 illustrates BLI titration data for PSMA Fab binding to peptide-283.

FIG. 95 illustrates BLI titration data for PSMA Fab binding to peptide-284.

FIG. 96 illustrates BLI titration data for PSMA Fab binding to peptide-285.

FIG. 97 illustrates BLI titration data for PSMA Fab binding to peptide-286.

FIG. 98 illustrates the core sequence motif of anti-PSMA Fab peptide-180 sequences generated using Weblogo 3.7.12.

FIG. 99 illustrates titration data for PSMA binding by peptide complexes of the present disclosure.

FIG. 100 illustrates titration data for PSMA binding by peptide complexes of the present disclosure following treatment with matrix metalloproteinase 9.

FIG. 101 illustrates titration data for CD28 binding by peptide complexes of the present disclosure.

FIG. 102 illustrates titration data for CD28 binding by peptide complexes of the present disclosure following treatment with matrix metalloproteinase 9.

### DETAILED DESCRIPTION

Multispecific antibodies combine the benefits of different binding specificities derived from two or more antibodies into a single composition. Multispecific antibodies, such as T-cell engagers (TCEs) for redirecting T cells to cancers have shown promise in both pre-clinical and clinical studies. This approach relies on binding of one antigen interacting portion of the antibody to a tumor-associated antigen or marker, while a second antigen interacting portion can bind to an effector cell antigen on a T cell, such as cluster of differentiation 3 (CD3), which then triggers cytotoxic activity. While TCEs have displayed potent anti-tumor activity in some cancers. TCE therapeutics may face challenges such as off-target toxicity in healthy tissue and slow clearance.

One such tumor-associated antigen is PSMA. Prostate-specific membrane antigen (PSMA), also known as glutamate carboxypeptidase II (GCPII), N-acetyl-L-aspartyl-L-glutamate peptidase I (NAALADasc I), or NAAG peptidase is an enzyme that in humans is encoded by the FOLH1 (folate hydrolase 1) gene. PSMA is a zinc metalloenzyme that resides in membranes. Most of the enzyme resides in the extracellular space. Human PSMA is highly expressed in the prostate, roughly a hundred times greater than in most other tissues. In some prostate cancers, PSMA is the second-most upregulated gene product, with an 8- to 12-fold increase over levels in noncancerous prostate cells.

Activation of T cells is a highly regulated process that typically requires two signaling events for full functionality: the first signal (signal 1) is initiated upon binding of the major histocompatibility complex (MHC)-antigen complex, which helps distinguish “self” from “non-self” to the T cell receptor (TCR), and the second costimulatory signal (signal 2) occurs through activation of a costimulatory receptor, such as CD28. While the first signal activates a T cell and triggers T cell mediated toxicity of the recognized cell, if the T cell does not receive a second costimulatory signal it can lead to T cell tolerance whereby the T cells continue to recognize the tumor antigen but do not mount an immune response against the tumor cell. The second costimulatory signal prevents T cell tolerance, and further activates the T cell to enhance T cell cytotoxicity towards the targeted cell. CD3-targeted T cell engagers (TCE) create signal 1 through TCE signaling without the need for a TCR/MHC interaction.

Disclosed herein are multispecific antibodies comprising a CD28 binding domain and a PSMA binding domain. Applicant has found that costimulation with the multispecific antibodies of the present disclosure enhances CD3-targeted TCE cytolytic activity. Peptide masks that impair binding of the CD28 binding domain to CD28 and peptide masks that impair binding of the PSMA binding domain to PSMA are attached to the multispecific antibodies via cleavable linkers to block CD28-mediated costimulation. Selective cleavage of the cleavable linkers by tumor-specific proteases of the tumor microenvironement restores the CD28 costimulatory activity of the multispecific antibodies to prevent off-target toxicity in healthy tissue. A half-life extending moiety is attached to the multispecific antibodies via the cleavable linker such that cleavage of the cleavable linker by tumor-specific proteases releases the half-life extending moiety to promote rapid clearance. The use tumor protease-cleavable linkers provides the benefit of targeted costimulatory activity in tumor microenvironments while minimizing off-target effects in healthy, nontumorous tissue.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzvmology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CHI) of the heavy chain. Fab fragments differ from Fab′ fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “CrossFab configuration” as used herein is a Fab molecule wherein the variable domains or the constant domains of the Fab heavy and light chain are exchanged (replaced by each other) (see, e.g., WO2009/080252A 1 and WO2017/055388A2). For example, a Fab molecule in a CrossFab configuration may comprise a peptide chain composed of the variable light chain domain and the heavy chain constant domain (VL-CH1 in an N- to C-terminal direction), and a peptide chain composed of the variable heavy chain domain and the light chain constant domain (VH-CL in an N- to C-terminal direction).

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL1 or vice versa. This protein retains the specificity of the original antibody, despite removal of the constant regions and the introduction of the linker, scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

The term “multispecific” means that the antibody is able to specifically bind to two or more distinct antigenic determinants for example two or more binding sites each formed by a pair of an antibody heavy chain variable domain (VH) and an antibody light chain variable domain (VL), or in the case of a single domain antibody a single variable domain, binding to different antigens.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L 1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L₃, and FR-L₄). The precise amino acid sequence boundaries of a given CDR or FR can be readily determined using any of a number of well-known schemes, including those described by Kabat et al. (1991), “Sequences of Proteins of Immunological Interest.” 5th Ed. Public Health Service, National Institutes of Health, Bethesda, MD (“Kabat” numbering scheme), Al-Lazikani et al., (1997) JMB 273,927-948 (“Chothia” numbering scheme): MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 Jan;27(1):55-77 (“IMGT” numbering scheme); Honegger A and Pluckthun A, “Yet another numbering scheme for immunoglobulin variable domains: an automatic modeling and analysis tool,” J Mol Biol, 2001 Jan 8;309(3):657-70, (“Aho” numbering scheme); and Whitelegg N R and Rees A R. “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

### Isolated Polypeptide or Polypeptide Complexes

Disclosed herein, in some embodiments, are isolated polypeptide or polypeptide complexes that comprise a first antigen recognizing molecule that binds to CD28 and a second antigen recognizing molecule that binds to prostate-specific membrane antigen (PSMA). In some embodiments, the first antigen recognizing molecule that binds to CD28 comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6. In some embodiments, the anti-CD28 heavy chain variable domain comprises an amino acid sequence with at least 80%, 85%, 90%, 95%, 98%, 99%, or 100% sequence identity to SEQ ID NO: 7, and the anti-CD28 light chain variable domain comprises an amino acid sequence with at least 80%, 85%, 90%, 95%, 98%, 99%, or 100% sequence identity to SEQ ID NO: 8. In some embodiments, the anti-CD28 heavy chain variable domain comprises the amino acid sequence of SEQ ID NO: 7, and the anti-CD28 light chain variable domain comprises the amino acid sequence of SEQ ID NO: 8.

TABLE 1
anti-CD28 sequences. CDR sequences are underlined and
were determined using IMGT definition.

| Construct | | SEQ ID |
|---|---|---|
| Description | Amino Acid Sequence | NO: |
| anti-CD28: HC: CDR1 | GYTFTSYY | 1 |
| anti-CD28: HC: CDR2 | IYPGNVNT | 2 |
| anti-CD28: HC: CDR3 | TRSHYGLDWNFDV | 3 |
| anti-CD28: LC: CDR1 | QNIYVW | 4 |
| anti-CD28: LC: CDR2 | KAS | |
| anti-CD28: LC: CDR3 | QQGQTYPYT | 6 |
| anti-CD28: HC | QVQLVQSGAEVKKPGASVKVSCKASGYTFTSYYI | 7 |
| | HWVRQAPGQGLEWIGSIYPGNVNTNYNEKFKDRA | |
| | TLTVDTSISTAYMELSRLRSDDTAVYFCTRSHYG | |
| | LDWNFDVWGQGTTVTVSS | |
| anti-CD28: LC | DIQMTQSPSSLSASVGDRVTITCHASQNIYVWLN | 8 |
| | WYQQKPGKAPKLLIYKASNLHTGVPSRFSGSGSG | |
| | TDFTLTISSLQPEDFATYYCQQGQTYPYTFGGGT | |
| | KVEIK | |
| Anti-CD28 scFv | QVQLVQSGAEVKKPGASVKVSCKASGYTFTSYYI | 9 |
| (VH - linker 1 - VL) | HWVRQAPGQGLEWIGSIYPGNVNTNYNEKFKDRA | |
| | TLTVDTSISTAYMELSRLRSDDTAVYFCTRSHYG | |
| | LDWNFDVWGQGTTVTVSSGGGGSGGGGSGGGGSD | |
| | IQMTQSPSSLSASVGDRVTITCHASQNIYVWLNW | |
| | YQQKPGKAPKLLIYKASNLHTGVPSRFSGSGSGT | |
| | DFTLTISSLQPEDFATYYCQQGQTYPYTFGGGTK | |
| | VEIK | |
| Anti-CD28 scFv | QVQLVQSGAEVKKPGASVKVSCKASGYTFTSYYI | 10 |
| (VH - linker 1 - VL) | HWVRQAPGQCLEWIGSIYPGNVNTNYNEKFKDRA | |
| Disulfide mutation: | TLTVDTSISTAYMELSRLRSDDTAVYFCTRSHYG | |
| G44C, G235C | LDWNFDVWGQGTTVTVSSGGGGSGGGGSGGGGSD | |
| | IQMTQSPSSLSASVGDRVTITCHASQNIYVWLNW | |
| | YQQKPGKAPKLLIYKASNLHTGVPSRFSGSGSGT | |
| | DFTLTISSLQPEDFATYYCQQGQTYPYTFGCGTK | |
| | VEIK | |
| Anti-CD28 scFv | DIQMTQSPSSLSASVGDRVTITCHASQNIYVWLN | 11 |
| (VL - linker 1 - VH) | WYQQKPGKAPKLLIYKASNLHTGVPSRFSGSGSG | |
| | TDFTLTISSLQPEDFATYYCQQGQTYPYTFGGGT | |
| | KVEIKGGGGSGGGGSGGGGSQVQLVQSGAEVKKP | |
| | GASVKVSCKASGYTFTSYYIHWVRQAPGQGLEWI | |
| | GSIYPGNVNTNYNEKFKDRATLTVDTSISTAYME | |
| | LSRLRSDDTAVYFCTRSHYGLDWNFDVWGQGTTV | |
| | TVSS | |
| Anti-CD28 scFv | DIQMTQSPSSLSASVGDRVTITCHASQNIYVWLN | 12 |
| (VL - linker 1 - VH) | WYQQKPGKAPKLLIYKASNLHTGVPSRFSGSGSG | |
| Disulfide mutation: | TDFTLTISSLQPEDFATYYCQQGQTYPYTFGCGT | |
| G100C, G166C | KVEIKGGGGSGGGGSGGGGSQVQLVQSGAEVKKP | |
| | GASVKVSCKASGYTFTSYYIHWVRQAPGQCLEWI | |
| | GSIYPGNVNTNYNEKFKDRATLTVDTSISTAYME | |
| | LSRLRSDDTAVYFCTRSHYGLDWNFDVWGQGTTV | |
| | TVSS | |
| Anti-CD28 Fab LC | DIQMTQSPSSLSASVGDRVTITCHASQNIYVWLN | 696 |
| | WYQQKPGKAPKLLIYKASNLHTGVPSRFSGSGSG | |
| | TDFTLTISSLQPEDFATYYCQQGQTYPYTFGGGT | |
| | KVEIKRTVAAPSVFIFPPSDEQLKSGTASVVCLL | |
| | NNFYPREAKVQWKVDNALQSGNSQESVTEQDSKD | |
| | STYSLSSTLTLSKADYEKHKVYACEVTHQGLSSP | |
| | VTKSFNRGEC | |
| Anti-CD28 Fab HC | QVQLVQSGAEVKKPGASVKVSCKASGYTFTSYYI | 697 |
| | HWVRQAPGQGLEWIGSIYPGNVNTNYNEKFKDRA | |
| | TLTVDTSISTAYMELSRLRSDDTAVYFCTRSHYG | |
| | LDWNFDVWGQGTTVTVSSASTKGPSVFPLAPSSK | |
| | STSGGTAALGCLVKDYFPEPVTVSWNSGALTSGV | |
| | HTFPAVLQSSGLYSLSSVVTVPSSSLGTQTYICN | |
| | VNHKPSNTKVDKKVEPKSC | |
| Anti-CD28 CrossFab | DIQMTQSPSSLSASVGDRVTITCHASQNIYVWLN | 698 |
| LC | WYQQKPGKAPKLLIYKASNLHTGVPSRFSGSGSG | |
| LC mutations: | TDFTLTISSLQPEDFATYYCQQGQTYPYTFGGGT | |
| SS added | KVEIKSSASTKGPSVFPLAPSSKSTSGGTAALGC | |
| | LVKDYFPEPVTVSWNSGALTSGVHTFPAVLQSSG | |
| | LYSLSSVVTVPSSSLGTQTYICNVNHKPSNTKVD | |
| | KKVEPKSC | |
| Anti-CD28 CrossFab | QVQLVQSGAEVKKPGASVKVSCKASGYTFTSYYI | 699 |
| HC | HWVRQAPGQGLEWIGSIYPGNVNTNYNEKFKDRA | |
| HC mutations: | TLTVDTSISTAYMELSRLRSDDTAVYFCTRSHYG | |
| AS replaced RT | LDWNFDVWGQGTTVTVSSASVAAPSVFIFPPSDE | |
| | QLKSGTASVVCLLNNFYPREAKVQWKVDNALQSG | |
| | NSQESVTEQDSKDSTYSLSSTLTLSKADYEKHKV | |
| | YACEVTHQGLSSPVTKSFNRGEC | |
| Anti-CD28 CrossFab | DIQMTQSPSSLSASVGDRVTITCHASQNIYVWLN | 700 |
| EE/RK LC | WYQQKPGKAPKLLIYKASNLHTGVPSRFSGSGSG | |
| LC mutations: | TDFTLTISSLQPEDFATYYCQQGQTYPYTFGGGT | |
| SS added; K139E; | KVEIKSSASTKGPSVFPLAPSSKSTSGGTAALGC | |
| K205E | LVEDYFPEPVTVSWNSGALTSGVHTFPAVLQSSG | |
| | LYSLSSVVTVPSSSLGTQTYICNVNHKPSNTKVD | |
| | EKVEPKSC | |
| Anti-CD28 CrossFab | QVQLVQSGAEVKKPGASVKVSCKASGYTFTSYYI | 701 |
| EE/RK HC | HWVRQAPGQGLEWIGSIYPGNVNTNYNEKFKDRA | |
| HC mutations: | TLTVDTSISTAYMELSRLRSDDTAVYFCTRSHYG | |
| AS replaced RT; | LDWNFDVWGQGTTVTVSSASVAAPSVFIFPPSDR | |
| E136R; Q137K | KLKSGTASVVCLLNNFYPREAKVQWKVDNALQSG | |
| | NSQESVTEQDSKDSTYSLSSTLTLSKADYEKHKV | |
| | YACEVTHQGLSSPVTKSFNRGEC | |

In some embodiments, the anti-CD28 antibody comprises a single chain variable fragment (scFv), a single domain antibody, a Fab, a Fab′, or a CrossFab. In some embodiments, the anti-CD28 antibody comprises the scFv. In some embodiments, the anti-CD28 antibody comprises a single chain variable fragment (scFv) with an engineered disulfide. In some embodiments, the anti-CD28 antibody comprises the scFv, and the scFv comprises an amino acid sequence with at least 80%, 85%, 90%, 95%, 98%, 990, or 100% sequence identity to SEQ ID NO: 9. In some embodiments, the anti-CD28 antibody comprises the scFv, and the scFv comprises the amino acid sequence of SEQ ID NO: 9. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 10. In some embodiments, the anti-CD28 antibody comprises the scFv, and the scFv comprises the amino acid sequence of SEQ ID NO: 10. In some embodiments, the anti-CD28 antibody comprises the scFv, and the scFv comprises an amino acid sequence with at least 80%, 85%, 90%, 95%, 98/a, 99%, or 100% sequence identity to SEQ ID NO: 11. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 12. In some embodiments, the anti-CD28 antibody comprises the scFv, and the scFv comprises the amino acid sequence of SEQ ID NO: 12.

In some embodiments, the anti-CD28 antibody comprises the Fab. In some embodiments, the Fab comprises an amino acid sequence with at least 80%, 85%, 99%, 95%, 98%, 99%, or 100% sequence identity to SEQ ID NO: 696 and an amino acid sequence with at least 80%, 85%, 90%, 95%, 98%, 99%, or 100% sequence identity to SEQ ID NO: 697. In some embodiments, the anti-CD28 antibody comprises the CrossFab. In some embodiments, the CrossFab comprises an amino acid sequence with at least 80%, 85%, 90%, 95%, 98%, 99%, or 100% sequence identity to SEQ ID NO: 698 and an amino acid sequence with at least 80%, 85%, 90%, 95%, 98%, 99%, or 100% sequence identity to SEQ ID NO: 699. In some embodiments, the CrossFab comprises an amino acid sequence with at least 80%, 85%, 90%, 95%, 98%, 99%, or 100% sequence identity to SEQ ID NO: 700 and an amino acid sequence with at least 80%, 85%, 90%, 95%, 98%, 99%, or 100% sequence identity to SEQ ID NO: 701. 11881 In some embodiments, the second antigen recognizing molecule that binds to PSMA comprises an anti-PSMA antibody. In some embodiments, the anti-PSMA antibody comprises an anti-PSMA heavy chain variable domain that comprises complementarity determining regions (CDRs): HC-CDR1, HC-CDR2, and HC-CDR3, wherein the HC-CDR, the HC-CDR2, and the HC-CDR3 comprise amino acid sequences of HC-CDR: SEQ ID NO: 13, HC-CDR2: SEQ ID NO: 14, and HC-CDR3: SEQ ID NO: 15 and the anti-PSMA antibody comprises an an-PSMA light chain variable domain that comprises complementarity determining regions (CDRs): LC-CDR1, LC-CDR2, and LC-CDR6, wherein the LC-CDR1, the LC-CDR2, and the LC-CDR3 of the light chain variable domain comprise an amino acid sequence of LC-CDR 1: SEQ ID NO: 16, LC-CDR2: (EA), and LC-CDR3: SEQ ID NO: 18. In some embodiments, the anti-PSMA heavy chain variable domain comprises an amino acid sequence with at least 80%, 85%, 90%, 95%, 98%, 99%, or 100% sequence identity to SEQ ID NO: 20, and the an-PSMA light chain variable domain comprises an amino acid sequence with at least 80%, 85%, 90%, 95%, 98%, 99%, or 100% sequence identity to SEQ ID NO: 19. In some embodiments, the anti-PSMA heavy chain variable domain comprises the amino acid sequence of SEQ ID NO: 20, and the anti-PSMA light chain variable domain comprises the amino acid sequence of NSEQ ID NO: 19.

TABLE 2
anti-PSMA sequences. CDR sequences are underlined and
were determined using IMGT definition.

| Construct | | SEQ ID |
|---|---|---|
| Description | Amino Acid Sequence | NO: |
| PSMA: HC: CDR1 | GFAFSRYG | 13 |
| PSMA: HC: CDR2 | IWYDGSNK | 14 |
| PSMA: HC: CDR3 | ARGGDFLYYYYYGMDV | 15 |
| PSMA: LC: CDR1 | QGISNY | 16 |
| PSMA: LC: CDR2 | EA | |
| PSMA: LC: CDR3 | QNYNSAPFT | 18 |
| 006 PSMA Fab LC | DIQMTQSPSSLSASVGDRVTITCRASQGISNYLAWYQQKT | 19 |
| | GKVPKFLIYEASTLQSGVPSRFSGGGSGTDFTLTISSLQP | |
| | EDVATYYCQNYNSAPFTFGPGTKVDIKRTVAAPSVFIFPP | |
| | SDEQLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |
| 006 PSMA Fab HC | QVQLVESGGGVVQPGRSLRLSCAASGFAFSRYGMHWVRQA | 20 |
| | PGKGLEWVAVIWYDGSNKYYADSVKGRFTISRDNSKNTQY | |
| | LQMNSLRAEDTAVYYCARGGDFLYYYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSLG | |
| | TQTYICNVNHKPSNTKVDKKVEPKSC | |
| PSMA Fab LC | DIQMTQSPSSLSASVGDRVTITCRASQGISNYLAWYQQKT | 702 |
| R19S mutant | GKVPKFLIYEASTLQSGVPSRFSGGGSGTDFTLTISSLQP | |
| | EDVATYYCQNYNSAPFTFGPGTKVDIKRTVAAPSVFIFPP | |
| | SDEQLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |
| PSMA Fab HC | QVQLVESGGGVVQPGRSLSLSCAASGFAFSRYGMHWVRQA | 703 |
| R19S mutant | PGKGLEWVAVIWYDGSNKYYADSVKGRFTISRDNSKNTQY | |
| R19S | LQMNSLRAEDTAVYYCARGGDFLYYYYYGMDVWGQGTTVT | |
| | VSSASTKGPSVFPLAPSSKSTSGGTAALGCLVKDYFPEPV | |
| | TVSWNSGALTSGVHTFPAVLQSSGLYSLSSVVTVPSSSLG | |
| | TQTYICNVNHKPSNTKVDKKVEPKSC | |
| PSMA Fab LC | DIQMTQSPSSLSASVGDRVTITCRASQGISNYLAWYQQKT | 704 |
| EE/RK mutant | GKVPKFLIYEASTLQSGVPSRFSGGGSGTDFTLTISSLQP | |
| E123R; Q124K | EDVATYYCQNYNSAPFTFGPGTKVDIKRTVAAPSVFIFPP | |
| | SDRKLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |
| PSMA Fab HC | QVQLVESGGGVVQPGRSLSLSCAASGFAFSRYGMHWVRQA | 705 |
| EE/RK mutant | PGKGLEWVAVIWYDGSNKYYADSVKGRFTISRDNSKNTQY | |
| K153E; K219E | LQMNSLRAEDTAVYYCARGGDFLYYYYYGMDVWGQGTTVT | |
| | VSSASTKGPSVFPLAPSSKSTSGGTAALGCLVEDYFPEPV | |
| | TVSWNSGALTSGVHTFPAVLQSSGLYSLSSVVTVPSSSLG | |
| | TQTYICNVNHKPSNTKVDEKVEPKSC | |

In some embodiments, the anti-PSMA antibody comprises a single chain variable fragment (scFv), a single domain antibody, a Fab, a Fab′, or a CrossFab. In some embodiments, the anti-PSMA antibody comprises the Fab or Fab′. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 19 and an amino acid sequence with at least at least 80%, 85%, 90%, 95%, 98%, 99%, or 100% sequence identity to SEQ ID NO: 20. In some embodiments, the anti-PSMA antibody comprises the Fab or Fab′, and the Fab or Fab′ comprises the amino acid sequences of SEQ ID NO: 19 and SEQ ID NO: 20. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 702 and an amino acid sequence with at least at least 80%, 85%, 90%, 95%, 98%, 99%, or 100% sequence identity to SEQ ID NO: 703. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 704 and an amino acid sequence with at least at least 80%, 85%, 90%, 95%, 98%, 99%, or 100% sequence identity to SEQ ID NO: 705.

In some embodiments, the anti-CD28 antibody comprises a scFv and the anti-PSMA antibody comprises a Fab or Fab′. In some embodiments, the scFv and the Fab or Fab′ are connected through a linker and the Fab or Fab′ comprise a Fab heavy chain polypeptide and a Fab light chain polypeptide. In some embodiments, the linker connects the scFv to the Fab heavy chain polypeptide. In some embodiments, the linker connects the scFv to the Fab light chain polypeptide. In some embodiments, the linker connects the scFv to the N-term of the Fab heavy chain polypeptide. In some embodiments, the linker connects the scFv to the C-term of the Fab heavy chain polypeptide. In some embodiments, the linker connects the scFv to the N-term of the Fab light chain polypeptide. In some embodiments, the linker connects the scFv to the C-term of the Fab light chain polypeptide. In some embodiments, the linker connects the anti-CD28 heavy chain variable domain of the scFv to the Fab heavy chain polypeptide. In some embodiments, the linker connects the anti-CD28 heavy chain variable domain of the scFv to the Fab light chain polypeptide.

In some embodiments, the linker connects the anti-CD28 heavy chain variable domain of the scFv to the N-term of the Fab heavy chain polypeptide. In some embodiments, the linker connects the anti-CD28 heavy chain variable domain of the scFv to the C-term of the Fab heavy chain polypeptide. In some embodiments, the linker connects the anti-CD28 heavy chain variable domain of the scFv to the N-term of the Fab light chain polypeptide. In some embodiments, the linker connects the anti-CD28 heavy chain variable domain of the scFv to the C-term of the Fab light chain polypeptide. In some embodiments, the linker connects the anti-CD28 light chain variable domain of the scFv to the Fab heavy chain polypeptide. In some embodiments, the linker connects the anti-CD28 light chain variable domain of the scFv to the Fab light chain polypeptide. In some embodiments, the linker connects the anti-CD28 light chain variable domain of the scFv to the N-term of the Fab heavy chain polypeptide. In some embodiments, the linker connects the anti-CD28 light chain variable domain of the scFv to the C-term of the Fab heavy chain polypeptide. In some embodiments, the linker connects the anti-CD28 light chain variable domain of the scFv to the N-term of the Fab light chain polypeptide. In some embodiments, the linker connects the anti-CD28 light chain variable domain of the scFv to the C-term of the Fab light chain polypeptide.

In some embodiments, the anti-CD28 antibody comprises a Fab or Fab′ and the anti-PSMA antibody comprises a Fab or Fab′. In some embodiments, the Fab or Fab′ of the anti-CD28 antibody and the Fab or Fab′ of the anti-PSMA antibody are connected through a linker and the Fab or Fab′ of the anti-PSMA antibody comprise a Fab heavy chain polypeptide and a Fab light chain polypeptide. In some embodiments, the linker connects the Fab or Fab′ of the anti-CD28 antibody to the Fab heavy chain polypeptide of the anti-PSMA antibody. In some embodiments, the linker connects the Fab or Fab′ of the anti-CD28 antibody to the Fab light chain polypeptide of the anti-PSMA antibody. In some embodiments, the linker connects the Fab or Fab′ of the anti-CD28 antibody to the N-term of the Fab heavy chain polypeptide of the anti-PSMA antibody. In some embodiments, the linker connects the Fab or Fab′ of the anti-CD28 antibody to the C-term of the Fab heavy chain polypeptide of the anti-PSMA antibody. In some embodiments, the linker connects the Fab or Fab′ of the anti-CD28 antibody to the N-term of the Fab light chain polypeptide of the anti-PSMA antibody. In some embodiments, the linker connects the Fab or Fab′ of the anti-CD28 antibody to the C-term of the Fab light chain polypeptide of the anti-PSMA antibody. In some embodiments, the linker connects a Fab heavy chain polypeptide of the anti-CD28 antibody to the Fab heavy chain polypeptide of the anti-PSMA antibody. In some embodiments, the linker connects a Fab heavy chain polypeptide of the anti-CD28 antibody to the Fab light chain polypeptide of the anti-PSMA antibody. In some embodiments, the linker connects a Fab heavy chain polypeptide of the anti-CD28 antibody to the N-term of the Fab heavy chain polypeptide of the anti-PSMA antibody. In some embodiments, the linker connects a Fab heavy chain polypeptide of the anti-CD28 antibody to the C-term of the Fab heavy chain polypeptide of the anti-PSMA antibody. In some embodiments, the linker connects a Fab heavy chain polypeptide of the anti-CD28 antibody to the N-term of the Fab light chain polypeptide of the anti-PSMA antibody. In some embodiments, the linker connects a Fab heavy chain polypeptide of the anti-CD28 antibody to the C-term of the Fab light chain polypeptide of the anti-PSMA antibody. In some embodiments, the linker connects a Fab light chain polypeptide of the anti-CD28 antibody to the Fab heavy chain polypeptide of the anti-PSMA antibody. In some embodiments, the linker connects a Fab light chain polypeptide of the anti-CD28 antibody to the Fab light chain polypeptide of the anti-PSMA antibody. In some embodiments, the linker connects a Fab light chain polypeptide of the anti-CD28 antibody to the N-term of the Fab heavy chain polypeptide of the anti-PSMA antibody. In some embodiments, the linker connects a Fab light chain polypeptide of the anti-CD28 antibody to the N-term of the Fab light chain polypeptide of the anti-PSMA antibody. In some embodiments, the linker connects a Fab light chain polypeptide of the anti-CD28 antibody to the C-term of the Fab light chain polypeptide of the anti-PSMA antibody.

In some embodiments, the anti-CD28 antibody comprises a CrossFab and the anti-PSMA antibody comprises a Fab or Fab′. In some embodiments, the CrossFab and the Fab or Fab′ are connected through a linker and the Fab or Fab′ of the anti-PSMA antibody comprise a Fab heavy chain polypeptide and a Fab light chain polypeptide. In some embodiments, the linker connects the CrossFab to the Fab heavy chain polypeptide of the anti-PSMA antibody. In some embodiments, the linker connects the CrossFab to the Fab light chain polypeptide of the anti-PSMA antibody. In some embodiments, the linker connects the CrossFab to the N-term of the Fab heavy chain polypeptide of the anti-PSMA antibody. In some embodiments, the linker connects the CrossFab to the C-term of the Fab heavy chain polypeptide of the anti-PSMA antibody. In some embodiments, the linker connects the CrossFab to the N-term of the Fab light chain polypeptide of the anti-PSMA antibody. In some embodiments, the linker connects the CrossFab to the C-term of the Fab light chain polypeptide of the anti-PSMA antibody. In some embodiments, the linker connects a Fab heavy chain polypeptide of the CrossFab to the Fab heavy chain polypeptide of the anti-PSMA antibody. In some embodiments, the linker connects a Fab heavy chain polypeptide of the CrossFab to the Fab light chain polypeptide of the anti-PSMA antibody. In some embodiments, the linker connects a Fab heavy chain polypeptide of the CrossFab to the N-term of the Fab heavy chain polypeptide of the anti-PSMA antibody. In some embodiments, the linker connects a Fab heavy chain polypeptide of the CrossFab to the C-term of the Fab heavy chain polypeptide of the anti-PSMA antibody. In some embodiments, the linker connects a Fab heavy chain polypeptide of the Cross-Fab to the N-term of the Fab light chain polypeptide of the anti-PSMA antibody. In some embodiments, the linker connects a Fab heavy chain polypeptide of the CrossFab to the C-term of the Fab light chain polypeptide of the anti-PSMA antibody. In some embodiments, the linker connects a Fab light chain polypeptide of the CrossFab to the Fab heavy chain polypeptide of the anti-PSMA antibody. In some embodiments, the linker connects a Fab light chain polypeptide of the CrossFab to the Fab light chain polypeptide of the anti-PSMA antibody. In some embodiments, the linker connects a Fab light chain polypeptide of the CrossFab to the N-term of the Fab heavy chain polypeptide of the anti-PSMA antibody. In some embodiments, the linker connects a Fab light chain polypeptide of the CrossFab to the C-term of the Fab heavy chain polypeptide of the anti-PSMA antibody. In some embodiments, the linker connects a Fab light chain polypeptide of the CrossFab to the N-term of the Fab light chain polypeptide of the anti-PSMA antibody. In some embodiments, the linker connects a Fab light chain polypeptide of the CrossFab to the C-term of the Fab light chain polypeptide of the anti-PSMA antibody.

In some embodiments, the anti-CD28 antibody comprises a Fab or Fab′ and the anti-PSMA antibody comprises a CrossFab.

In some embodiments, the linker is at least 5 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker is 15 amino acids in length. In some embodiments, the linker comprises an amino acid sequence of SEQ ID NO: 21 (GGGGSGGGGSGGGGS) or SEQ ID NO: 22 (GGGGS).

TABLE 3
Linker sequences

| Construct | Amino Acid | SEQ ID |
|---|---|---|
| Description | Sequence | NO: |
| Linker 1 | GGGGSGGGGSGGGGS | 21 |
| Linker 2 | GGGGS | 22 |

Polypeptide Sequences that Bind to CD28 and PSMA

In some embodiments, the isolated polypeptide or polypeptide complex comprises amino acid sequences with at least 80%, 85%, 90%, 95%, 98%, 99%, or 100% sequence identity to SEQ ID NO: 23 and SEQ ID NO: 24. In some embodiments, the isolated polypeptide or polypeptide complex comprises amino acid sequences with at least 80%, 85%, 90%, 95%, 98%, 99%, or 100% sequence identity to SEQ ID NO: 25 and SEQ ID NO: 26. In some embodiments, the isolated polypeptide or polypeptide complex comprises amino acid sequences with at least 80%, 85%, 90%, 95%, 98%, 99%, or 100% sequence identity to SEQ ID NO: 27 and SEQ ID NO: 28. In some embodiments, the isolated polypeptide or polypeptide complex comprises amino acid sequences with at least 80%, 85%, 90%, 95%, 98%, 99%, or 100% sequence identity to SEQ ID NO: 29 and SEQ ID NO: 30. In some embodiments, the isolated polypeptide or polypeptide complex comprises amino acid sequences with at least 80%, 85%, 90% 95%, 98%, 99%, or 100% sequence identity to SEQ ID NO: 31 and SEQ ID NO: 32. In some embodiments, the isolated polypeptide or polypeptide complex comprises the amino acid sequences of SEQ ID NO: 25 and SEQ ID NO: 26. In some embodiments, the isolated polypeptide or polypeptide complex comprises amino acid sequences with at least 80%, 85%, 90%, 95%, 98%, 99%, or 100% sequence identity to SEQ ID NO: 706, and SEQ ID NO: 707. In some embodiments, the isolated polypeptide or polypeptide complex comprises amino acid sequences with at least 80%, 85%, 90%, 95%, 98%, 99%, or 100% sequence identity to SEQ ID NO: 708, SEQ ID NO: 709, and SEQ ID NO: 710. In some embodiments, the isolated polypeptide or polypeptide complex comprises amino acid sequences with at least 80%, 85%, 90%, 95%, 98%, 99%, or 100% sequence identity to SEQ ID NO: 711, SEQ ID NO: 712, and SEQ ID NO: 713. In some embodiments, the isolated polypeptide or polypeptide complex comprises amino acid sequences with at least 80%, 85%, 90%, 95%, 98%, 99%, or 100% sequence identity to SEQ ID NO: 714, SEQ ID NO: 715, and SEQ ID NO: 716. In some embodiments, the isolated polypeptide or polypeptide complex comprises amino acid sequences with at least 80%, 85%, 90%, 95%, 98%, 99%, or 100% sequence identity to SEQ ID NO: 717, SEQ ID NO: 718, and SEQ ID NO: 719. In some embodiments, the isolated polypeptide or polypeptide complex comprises amino acid sequences with at least 80%, 85%, 90%, 95%, 98%, 99%, or 100% sequence identity to SEQ ID NO: 720, SEQ ID NO: 721, and SEQ ID NO: 722. In some embodiments, the isolated polypeptide or polypeptide complex comprises amino acid sequences with at least 80%, 85%, 90SEQ, 95%, 98%, 99% or 100% sequence identity to SEQ ID NO: 723, SEQ ID NO: 724, and SEQ ID NO: 725. In some embodiments, the isolated polypeptide or polypeptide complex comprises amino acid sequences with at least 80%, 85%, 90%, 95%, 98%, 99%, or 100% sequence identity to SEQ ID NO: 726, SEQ ID NO: 727, and SEQ ID NO: 728. In some embodiments, the isolated polypeptide or polypeptide complex comprises amino acid sequences with at least 80%, 85%, 90%, 95%, 98%, 99%, or 100% sequence identity to SEQ ID NO: 729, SEQ ID NO: 730, and SEQ ID NO: 731.

TABLE 4
Polypeptide Complexes that Bind to CD28 and PSMA

| Construct | | SEQ ID |
|---|---|---|
| Description | Amino Acid Sequence | NO: |
| Ab-1 (Fab LC) | DIQMTQSPSSLSASVGDRVTITCRASQGISNYL | 23 |
| wt CD28 scFv | AWYQQKTGKVPKFLIYEASTLQSGVPSRFSGGG | |
| (Vh-linker-VI) | SGTDFTLTISSLQPEDVATYYCQNYNSAPFTFG | |
| | PGTKVDIKRTVAAPSVFIFPPSDEQLKSGTASV | |
| | VCLLNNFYPREAKVQWKVDNALQSGNSQESVTE | |
| | QDSKDSTYSLSSTLTLSKADYEKHKVYACEVTH | |
| | QGLSSPVTKSFNRGEC | |
| Ab-1 (Fab HC) | QVQLVQSGAEVKKPGASVKVSCKASGYTFTSYY | 24 |
| wt CD28 scFv | IHWVRQAPGQGLEWIGSIYPGNVNTNYNEKFKD | |
| (Vh-linker-VI) | RATLTVDTSISTAYMELSRLRSDDTAVYFCTRS | |
| | HYGLDWNFDVWGQGTTVTVSSGGGGSGGGGSGG | |
| | GGSDIQMTQSPSSLSASVGDRVTITCHASQNIY | |
| | VWLNWYQQKPGKAPKLLIYKASNLHTGVPSRFS | |
| | GSGSGTDFTLTISSLQPEDFATYYCQQGQTYPY | |
| | TFGGGTKVEIKGGGGSQVQLVESGGGVVQPGRS | |
| | LRLSCAASGFAFSRYGMHWVRQAPGKGLEWVAV | |
| | IWYDGSNKYYADSVKGRFTISRDNSKNTQYLQM | |
| | NSLRAEDTAVYYCARGGDFLYYYYYGMDVWGQG | |
| | TTVTVSSASTKGPSVFPLAPSSKSTSGGTAALG | |
| | CLVKDYFPEPVTVSWNSGALTSGVHTFPAVLQS | |
| | SGLYSLSSVVTVPSSSLGTQTYICNVNHKPSNT | |
| | KVDKKVEPKSC | |
| Ab-2 (Fab LC) | QVQLVQSGAEVKKPGASVKVSCKASGYTFTSYY | 25 |
| wt CD28 scFv | IHWVRQAPGQGLEWIGSIYPGNVNTNYNEKFKD | |
| (Vh-linker-VI) | RATLTVDTSISTAYMELSRLRSDDTAVYFCTRS | |
| | HYGLDWNFDVWGQGTTVTVSSGGGGSGGGGSGG | |
| | GGSDIQMTQSPSSLSASVGDRVTITCHASQNIY | |
| | VWLNWYQQKPGKAPKLLIYKASNLHTGVPSRFS | |
| | GSGSGTDFTLTISSLQPEDFATYYCQQGQTYPY | |
| | TFGGGTKVEIKGGGGSDIQMTQSPSSLSASVGD | |
| | RVTITCRASQGISNYLAWYQQKTGKVPKFLIYE | |
| | ASTLQSGVPSRFSGGGSGTDFTLTISSLQPEDV | |
| | ATYYCQNYNSAPFTFGPGTKVDIKRTVAAPSVF | |
| | IFPPSDEQLKSGTASVVCLLNNFYPREAKVQWK | |
| | VDNALQSGNSQESVTEQDSKDSTYSLSSTLTLS | |
| | KADYEKHKVYACEVTHQGLSSPVTKSFNRGEC | |
| Ab-2 (Fab HC) | QVQLVESGGGVVQPGRSLRLSCAASGFAFSRYG | 26 |
| wt CD28 scFv | MHWVRQAPGKGLEWVAVIWYDGSNKYYADSVKG | |
| (Vh-linker-VI) | RFTISRDNSKNTQYLQMNSLRAEDTAVYYCARG | |
| | GDFLYYYYYGMDVWGQGTTVTVSSASTKGPSVF | |
| | PLAPSSKSTSGGTAALGCLVKDYFPEPVTVSWN | |
| | SGALTSGVHTFPAVLQSSGLYSLSSVVTVPSSS | |
| | LGTQTYICNVNHKPSNTKVDKKVEPKSC | |
| Ab-3 (Fab LC) | QVQLVQSGAEVKKPGASVKVSCKASGYTFTSYY | 27 |
| Disulfide CD28 scFv | IHWVRQAPGQCLEWIGSIYPGNVNTNYNEKFKD | |
| G235C | RATLTVDTSISTAYMELSRLRSDDTAVYFCTRS | |
| (Vh-linker V1) | HYGLDWNFDVWGQGTTVTVSSGGGGSGGGGSGG | |
| | GGSDIQMTQSPSSLSASVGDRVTITCHASQNIY | |
| | VWLNWYQQKPGKAPKLLIYKASNLHTGVPSRFS | |
| | GSGSGTDFTLTISSLQPEDFATYYCQQGQTYPY | |
| | TFGCGTKVEIKGGGGSDIQMTQSPSSLSASVGD | |
| | RVTITCRASQGISNYLAWYQQKTGKVPKFLIYE | |
| | ASTLQSGVPSRFSGGGSGTDFTLTISSLQPEDV | |
| | ATYYCQNYNSAPFTFGPGTKVDIKRTVAAPSVF | |
| | IFPPSDEQLKSGTASVVCLLNNFYPREAKVQWK | |
| | VDNALQSGNSQESVTEQDSKDSTYSLSSTLTLS | |
| | KADYEKHKVYACEVTHQGLSSPVTKSFNRGEC | |
| Ab-3 (Fab HC) | QVQLVESGGGVVQPGRSLRLSCAASGFAFSRYG | 28 |
| Disulfide CD28 scFv | MHWVRQAPGKGLEWVAVIWYDGSNKYYADSVKG | |
| G44C | RFTISRDNSKNTQYLQMNSLRAEDTAVYYCARG | |
| (Vh-linkerVI) | GDFLYYYYYGMDVWGQGTTVTVSSASTKGPSVF | |
| | PLAPSSKSTSGGTAALGCLVKDYFPEPVTVSWN | |
| | SGALTSGVHTFPAVLQSSGLYSLSSVVTVPSSS | |
| | LGTQTYICNVNHKPSNTKVDKKVEPKSC | |
| Ab-4 (Fab LC) | DIQMTQSPSSLSASVGDRVTITCHASQNIYVWL | 29 |
| wt CD28 scFv | NWYQQKPGKAPKLLIYKASNLHTGVPSRFSGSG | |
| (VI-linker-Vh) | SGTDFTLTISSLQPEDFATYYCQQGQTYPYTFG | |
| | GGTKVEIKGGGGSGGGGSGGGGSQVQLVQSGAE | |
| | VKKPGASVKVSCKASGYTFTSYYIHWVRQAPGQ | |
| | GLEWIGSIYPGNVNTNYNEKFKDRATLTVDTSI | |
| | STAYMELSRLRSDDTAVYFCTRSHYGLDWNFDV | |
| | WGQGTTVTVSSGGGGSDIQMTQSPSSLSASVGD | |
| | RVTITCRASQGISNYLAWYQQKTGKVPKFLIYE | |
| | ASTLQSGVPSRFSGGGSGTDFTLTISSLQPEDV | |
| | ATYYCQNYNSAPFTFGPGTKVDIKRTVAAPSVF | |
| | IFPPSDEQLKSGTASVVCLLNNFYPREAKVQWK | |
| | VDNALQSGNSQESVTEQDSKDSTYSLSSTLTLS | |
| | KADYEKHKVYACEVTHQGLSSPVTKSFNRGEC | |
| Ab-4 (Fab HC) | QVQLVESGGGVVQPGRSLRLSCAASGFAFSRYG | 30 |
| wt CD28 scFv | MHWVRQAPGKGLEWVAVIWYDGSNKYYADSVKG | |
| (VI-linker-Vh) | RFTISRDNSKNTQYLQMNSLRAEDTAVYYCARG | |
| | GDFLYYYYYGMDVWGQGTTVTVSSASTKGPSVF | |
| | PLAPSSKSTSGGTAALGCLVKDYFPEPVTVSWN | |
| | SGALTSGVHTFPAVLQSSGLYSLSSVVTVPSSS | |
| | LGTQTYICNVNHKPSNTKVDKKVEPKSC | |
| Ab-5 (Fab LC) | DIQMTQSPSSLSASVGDRVTITCHASQNIYVWL | 31 |
| Disulfide CD28 scFv | NWYQQKPGKAPKLLIYKASNLHTGVPSRFSGSG | |
| (VI-linker-Vh) | SGTDFTLTISSLQPEDFATYYCQQGQTYPYTFG | |
| G100C | CGTKVEIKGGGGSGGGGSGGGGSQVQLVQSGAE | |
| | VKKPGASVKVSCKASGYTFTSYYIHWVRQAPGQ | |
| | CLEWIGSIYPGNVNTNYNEKFKDRATLTVDTSI | |
| | STAYMELSRLRSDDTAVYFCTRSHYGLDWNFDV | |
| | WGQGTTVTVSSGGGGSDIQMTQSPSSLSASVGD | |
| | RVTITCRASQGISNYLAWYQQKTGKVPKFLIYE | |
| | ASTLQSGVPSRFSGGGSGTDFTLTISSLQPEDV | |
| | ATYYCQNYNSAPFTFGPGTKVDIKRTVAAPSVF | |
| | IFPPSDEQLKSGTASVVCLLNNFYPREAKVQWK | |
| | VDNALQSGNSQESVTEQDSKDSTYSLSSTLTLS | |
| | KADYEKHKVYACEVTHQGLSSPVTKSFNRGEC | |
| Ab-5 (Fab HC) | QVQLVESGGGVVQPGRSLRLSCAASGFAFSRYG | 32 |
| Disulfide CD28 scFv | MHWVRQAPGKGLEWVAVIWYDGSNKYYADSVKG | |
| (VI-linker-Vh) | RFTISRDNSKNTQYLQMNSLRAEDTAVYYCARG | |
| G166C | GDFLYYYYYGMDVWGQGTTVTVSSASTKGPSVF | |
| | PLAPSSKSTSGGTAALGCLVKDYFPEPVTVSWN | |
| | SGALTSGVHTFPAVLQSSGLYSLSSVVTVPSSS | |
| | LGTQTYICNVNHKPSNTKVDKKVEPKSC | |
| Ab-6 (Chain 1) | QVQLVQSGAEVKKPGASVKVSCKASGYTFTSYY | 706 |
| PSMA Fab HC: R19S | IHWVRQAPGQGLEWIGSIYPGNVNTNYNEKFKD | |
| | RATLTVDTSISTAYMELSRLRSDDTAVYFCTRS | |
| | HYGLDWNFDVWGQGTTVTVSSGGGGSGGGGSGG | |
| | GGSDIQMTQSPSSLSASVGDRVTITCHASQNIY | |
| | VWLNWYQQKPGKAPKLLIYKASNLHTGVPSRFS | |
| | GSGSGTDFTLTISSLQPEDFATYYCQQGQTYPY | |
| | TFGGGTKVEIKGGGGSDIQMTQSPSSLSASVGD | |
| | RVTITCRASQGISNYLAWYQQKTGKVPKFLIYE | |
| | ASTLQSGVPSRFSGGGSGTDFTLTISSLQPEDV | |
| | ATYYCQNYNSAPFTFGPGTKVDIKRTVAAPSVF | |
| | IFPPSDEQLKSGTASVVCLLNNFYPREAKVQWK | |
| | VDNALQSGNSQESVTEQDSKDSTYSLSSTLTLS | |
| | KADYEKHKVYACEVTHQGLSSPVTKSFNRGEC | |
| Ab-6 (Chain 2) | QVQLVESGGGVVQPGRSLSLSCAASGFAFSRYG | 707 |
| PSMA Fab HC: R19S | MHWVRQAPGKGLEWVAVIWYDGSNKYYADSVKG | |
| | RFTISRDNSKNTQYLQMNSLRAEDTAVYYCARG | |
| | GDFLYYYYYGMDVWGQGTTVTVSSASTKGPSVF | |
| | PLAPSSKSTSGGTAALGCLVKDYFPEPVTVSWN | |
| | SGALTSGVHTFPAVLQSSGLYSLSSVVTVPSSS | |
| | LGTQTYICNVNHKPSNTKVDKKVEPKSC | |
| Ab-7 (Chain 1) | QVQLVQSGAEVKKPGASVKVSCKASGYTFTSYY | 708 |
| PSMA Fab HC: R19S; | IHWVRQAPGQGLEWIGSIYPGNVNTNYNEKFKD | |
| CD28 LC: CrossFab SS | RATLTVDTSISTAYMELSRLRSDDTAVYFCTRS | |
| added, K139E, K205E; | HYGLDWNFDVWGQGTTVTVSSASVAAPSVFIFP | |
| CD28 HC: CrossFab AS | PSDRKLKSGTASVVCLLNNFYPREAKVQWKVDN | |
| replaced RT, E136R, Q137K | ALQSGNSQESVTEQDSKDSTYSLSSTLTLSKAD | |
| linker connects CHI(CD28 | YEKHKVYACEVTHQGLSSPVTKSFNRGEC | |
| crossFab EE/RK) -- VL(PSMA | | |
| Fab) | | |
| Ab-7 (Chain 2) | DIQMTQSPSSLSASVGDRVTITCHASQNIYVWL | 709 |
| PSMA Fab HC: R19S | NWYQQKPGKAPKLLIYKASNLHTGVPSRFSGSG | |
| CD28 LC: CrossFab SS | SGTDFTLTISSLQPEDFATYYCQQGQTYPYTFG | |
| added, K139E, K205E; | GGTKVEIKSSASTKGPSVFPLAPSSKSTSGGTA | |
| CD28 HC: CrossFab AS | ALGCLVEDYFPEPVTVSWNSGALTSGVHTFPAV | |
| replaced RT, E136R, Q137K | LQSSGLYSLSSVVTVPSSSLGTQTYICNVNHKP | |
| linker connects CHI(CD28 | SNTKVDEKVEPKSCDGGGGSGGGGSDIQMTQSP | |
| crossFab EE/RK) -- VL(PSMA | SSLSASVGDRVTITCRASQGISNYLAWYQQKTG | |
| Fab) | KVPKFLIYEASTLQSGVPSRFSGGGSGTDFTLT | |
| | ISSLQPEDVATYYCQNYNSAPFTFGPGTKVDIK | |
| | RTVAAPSVFIFPPSDEQLKSGTASVVCLLNNFY | |
| | PREAKVQWKVDNALQSGNSQESVTEQDSKDSTY | |
| | SLSSTLTLSKADYEKHKVYACEVTHQGLSSPVT | |
| | KSFNRGEC | |
| Ab-7 (Chain 3) | QVQLVESGGGVVQPGRSLSLSCAASGFAFSRYG | 710 |
| PSMA Fab HC: R19S | MHWVRQAPGKGLEWVAVIWYDGSNKYYADSVKG | |
| CD28 LC: CrossFab SS | RFTISRDNSKNTQYLQMNSLRAEDTAVYYCARG | |
| added, K139E, K205E; | GDFLYYYYYGMDVWGQGTTVTVSSASTKGPSVF | |
| CD28 HC: CrossFab AS | PLAPSSKSTSGGTAALGCLVKDYFPEPVTVSWN | |
| replaced RT, E136R, Q137K | SGALTSGVHTFPAVLQSSGLYSLSSVVTVPSSS | |
| linker connects CHI(CD28 | LGTQTYICNVNHKPSNTKVDKKVEPKSC | |
| crossFab EE/RK) -- VL(PSMA | | |
| Fab) | | |
| Ab-8 (Chain 1) | DIQMTQSPSSLSASVGDRVTITCHASQNIYVWL | 711 |
| PSMA Fab HC: R19S; | NWYQQKPGKAPKLLIYKASNLHTGVPSRFSGSG | |
| CD28 LC: CrossFab; SS | SGTDFTLTISSLQPEDFATYYCQQGQTYPYTFG | |
| added, K139E, K205E; | GGTKVEIKSSASTKGPSVFPLAPSSKSTSGGTA | |
| CD28 HC: CrossFab AS | ALGCLVEDYFPEPVTVSWNSGALTSGVHTFPAV | |
| replaced RT, E136R, Q137K; | LQSSGLYSLSSVVTVPSSSLGTQTYICNVNHKP | |
| linker connects CL(CD28 | SNTKVDEKVEPKSC | |
| crossFab EE/RK) -- VL(PSMA | | |
| Fab) | | |
| Ab-8 (Chain 2) | QVQLVQSGAEVKKPGASVKVSCKASGYTFTSYY | 712 |
| PSMA Fab HC: R19S; | IHWVRQAPGQGLEWIGSIYPGNVNTNYNEKFKD | |
| CD28 LC: CrossFab, SS | RATLTVDTSISTAYMELSRLRSDDTAVYFCTRS | |
| added, K139E, K205E; | HYGLDWNFDVWGQGTTVTVSSASVAAPSVFIFP | |
| CD28 HC: CrossFab AS | PSDRKLKSGTASVVCLLNNFYPREAKVQWKVDN | |
| replaced RT, E136R, Q137K; | ALQSGNSQESVTEQDSKDSTYSLSSTLTLSKAD | |
| linker connects CL(CD28 | YEKHKVYACEVTHQGLSSPVTKSFNRGECDGGG | |
| crossFab EE/RK) -- VL(PSMA | GSGGGGSDIQMTQSPSSLSASVGDRVTITCRAS | |
| Fab) | QGISNYLAWYQQKTGKVPKFLIYEASTLQSGVP | |
| | SRFSGGGSGTDFTLTISSLQPEDVATYYCQNYN | |
| | SAPFTFGPGTKVDIKRTVAAPSVFIFPPSDEQL | |
| | KSGTASVVCLLNNFYPREAKVQWKVDNALQSGN | |
| | SQESVTEQDSKDSTYSLSSTLTLSKADYEKHKV | |
| | YACEVTHQGLSSPVTKSFNRGEC | |
| Ab-8 (Chain 3) | QVQLVESGGGVVQPGRSLSLSCAASGFAFSRYG | 713 |
| PSMA Fab HC: R19S; | MHWVRQAPGKGLEWVAVIWYDGSNKYYADSVKG | |
| CD28 LC: CrossFab, SS | RFTISRDNSKNTQYLQMNSLRAEDTAVYYCARG | |
| added, K139E, K205E; | GDFLYYYYYGMDVWGQGTTVTVSSASTKGPSVF | |
| CD28 HC: CrossFab AS | PLAPSSKSTSGGTAALGCLVKDYFPEPVTVSWN | |
| replaced RT, E136R, Q137K; | SGALTSGVHTFPAVLQSSGLYSLSSVVTVPSSS | |
| linker connects CL(CD28 | LGTQTYICNVNHKPSNTKVDKKVEPKSC | |
| crossFab EE/RK) -- VL(PSMA | | |
| Fab) | | |
| Ab-9 (Chain 1) | DIQMTQSPSSLSASVGDRVTITCHASQNIYVWL | 714 |
| PSMA Fab HC: R19S | NWYQQKPGKAPKLLIYKASNLHTGVPSRFSGSG | |
| CD28 LC: CrossFab; SS | SGTDFTLTISSLQPEDFATYYCQQGQTYPYTFG | |
| added; K139E; K205E; | GGTKVEIKSSASTKGPSVFPLAPSSKSTSGGTA | |
| CD28 HC: CrossFab AS | ALGCLVEDYFPEPVTVSWNSGALTSGVHTFPAV | |
| replaced RT; E136R; Q137K; | LQSSGLYSLSSVVTVPSSSLGTQTYICNVNHKP | |
| linker connects CL(CD28 | SNTKVDEKVEPKSC | |
| crossFab EE/RK) -- | | |
| VH(PSMA Fab) | | |
| Ab-9 (Chain 2) | QVQLVQSGAEVKKPGASVKVSCKASGYTFTSYY | 715 |
| PSMA Fab HC: R19S | IHWVRQAPGQGLEWIGSIYPGNVNTNYNEKFKD | |
| CD28 LC: CrossFab; SS | RATLTVDTSISTAYMELSRLRSDDTAVYFCTRS | |
| added; K139E; K205E; | HYGLDWNFDVWGQGTTVTVSSASVAAPSVFIFP | |
| CD28HC: CrossFab AS | PSDRKLKSGTASVVCLLNNFYPREAKVQWKVDN | |
| replaced RT; E136R; Q137K; | ALQSGNSQESVTEQDSKDSTYSLSSTLTLSKAD | |
| linker connects CL(CD28 | YEKHKVYACEVTHQGLSSPVTKSFNRGECDGGG | |
| crossFab EE/RK) -- | GSGGGGSQVQLVESGGGVVQPGRSLSLSCAASG | |
| VH(PSMA Fab) | FAFSRYGMHWVRQAPGKGLEWVAVIWYDGSNKY | |
| | YADSVKGRFTISRDNSKNTQYLQMNSLRAEDTA | |
| | VYYCARGGDFLYYYYYGMDVWGQGTTVTVSSAS | |
| | TKGPSVFPLAPSSKSTSGGTAALGCLVKDYFPE | |
| | PVTVSWNSGALTSGVHTFPAVLQSSGLYSLSSV | |
| | VTVPSSSLGTQTYICNVNHKPSNTKVDKKVEPK | |
| | SC | |
| Ab-9 (Chain 3) | DIQMTQSPSSLSASVGDRVTITCRASQGISNYL | 716 |
| PSMA Fab HC: R19S | AWYQQKTGKVPKFLIYEASTLQSGVPSRFSGGG | |
| CD28 LC: CrossFab; SS | SGTDFTLTISSLQPEDVATYYCQNYNSAPFTFG | |
| added; K139E; K205E; | PGTKVDIKRTVAAPSVFIFPPSDEQLKSGTASV | |
| CD28HC: CrossFab AS | VCLLNNFYPREAKVQWKVDNALQSGNSQESVTE | |
| replaced RT; E136R; Q137K; | QDSKDSTYSLSSTLTLSKADYEKHKVYACEVTH | |
| linker connects CL(CD28 | QGLSSPVTKSFNRGEC | |
| crossFab EE/RK) -- | | |
| VH(PSMA Fab) | | |
| Ab-10 (Chain 1) | QVQLVQSGAEVKKPGASVKVSCKASGYTFTSYY | 717 |
| PSMA Fab HC: R19S | IHWVRQAPGQGLEWIGSIYPGNVNTNYNEKFKD | |
| CD28 LC: CrossFab; SS | RATLTVDTSISTAYMELSRLRSDDTAVYFCTRS | |
| added; K139E; K205E; | HYGLDWNFDVWGQGTTVTVSSASVAAPSVFIFP | |
| CD28 HC: CrossFab AS | PSDRKLKSGTASVVCLLNNFYPREAKVQWKVDN | |
| replaced RT; E136R; Q137K; | ALQSGNSQESVTEQDSKDSTYSLSSTLTLSKAD | |
| linker connects CHI(CD28 | YEKHKVYACEVTHQGLSSPVTKSFNRGEC | |
| crossFab EE/RK) -- | | |
| VH(PSMA Fab) | | |
| Ab-10 (Chain 2) | DIQMTQSPSSLSASVGDRVTITCHASQNIYVWL | 718 |
| PSMA Fab HC: R19S | NWYQQKPGKAPKLLIYKASNLHTGVPSRFSGSG | |
| CD28 LC: CrossFab; SS | SGTDFTLTISSLQPEDFATYYCQQGQTYPYTFG | |
| added; K139E; K205E; | GGTKVEIKSSASTKGPSVFPLAPSSKSTSGGTA | |
| CD28 HC: CrossFab AS | ALGCLVEDYFPEPVTVSWNSGALTSGVHTFPAV | |
| replaced RT; E136R; Q137K; | LQSSGLYSLSSVVTVPSSSLGTQTYICNVNHKP | |
| linker connects CHI(CD28 | SNTKVDEKVEPKSCDGGGGSGGGGSQVQLVESG | |
| crossFab EE/RK) -- | GGVVQPGRSLSLSCAASGFAFSRYGMHWVRQAP | |
| VH(PSMA Fab) | GKGLEWVAVIWYDGSNKYYADSVKGRFTISRDN | |
| | SKNTQYLQMNSLRAEDTAVYYCARGGDFLYYYY | |
| | YGMDVWGQGTTVTVSSASTKGPSVFPLAPSSKS | |
| | TSGGTAALGCLVKDYFPEPVTVSWNSGALTSGV | |
| | HTFPAVLQSSGLYSLSSVVTVPSSSLGTQTYIC | |
| | NVNHKPSNTKVDKKVEPKSC | |
| Ab-10 (Chain 3) | DIQMTQSPSSLSASVGDRVTITCRASQGISNYL | 719 |
| PSMA Fab HC: R19S | AWYQQKTGKVPKFLIYEASTLQSGVPSRFSGGG | |
| CD28 LC: CrossFab; SS | SGTDFTLTISSLQPEDVATYYCQNYNSAPFTFG | |
| added; K139E; K205E; | PGTKVDIKRTVAAPSVFIFPPSDEQLKSGTASV | |
| CD28 HC: CrossFab AS | VCLLNNFYPREAKVQWKVDNALQSGNSQESVTE | |
| replaced RT; E136R; Q137K; | QDSKDSTYSLSSTLTLSKADYEKHKVYACEVTH | |
| linker connects CHI(CD28 | QGLSSPVTKSFNRGEC | |
| crossFab EE/RK) -- | | |
| VH(PSMA Fab) | | |
| Ab-11 (Chain 1) | DIQMTQSPSSLSASVGDRVTITCHASQNIYVWL | 720 |
| PSMA Fab LC: E123R; | NWYQQKPGKAPKLLIYKASNLHTGVPSRFSGSG | |
| Q124K | SGTDFTLTISSLQPEDFATYYCQQGQTYPYTFG | |
| PSMA Fab HC: R19S; | GGTKVEIKSSASTKGPSVFPLAPSSKSTSGGTA | |
| K153E; K219E | ALGCLVKDYFPEPVTVSWNSGALTSGVHTFPAV | |
| CD28 LC: CrossFab; SS | LQSSGLYSLSSVVTVPSSSLGTQTYICNVNHKP | |
| added | SNTKVDKKVEPKSC | |
| CD28 HC: CrossFab; AS | | |
| replaced RT | | |
| linker connects CL(CD28 | | |
| crossFab) -- VL(PSMA Fab | | |
| EE/RK | | |
| Ab-11 (Chain 2) | QVQLVQSGAEVKKPGASVKVSCKASGYTFTSYY | 721 |
| PSMA Fab LC: E123R; | IHWVRQAPGQGLEWIGSIYPGNVNTNYNEKFKD | |
| Q124K | RATLTVDTSISTAYMELSRLRSDDTAVYFCTRS | |
| PSMA Fab HC: R19S; | HYGLDWNFDVWGQGTTVTVSSASVAAPSVFIFP | |
| K153E; K219E | PSDEQLKSGTASVVCLLNNFYPREAKVQWKVDN | |
| CD28 LC: CrossFab; SS | ALQSGNSQESVTEQDSKDSTYSLSSTLTLSKAD | |
| added | YEKHKVYACEVTHQGLSSPVTKSFNRGECDGGG | |
| CD28 HC: CrossFab; AS | GSGGGGSDIQMTQSPSSLSASVGDRVTITCRAS | |
| replaced RT | QGISNYLAWYQQKTGKVPKFLIYEASTLQSGVP | |
| linker connects CL(CD28 | SRFSGGGSGTDFTLTISSLQPEDVATYYCQNYN | |
| crossFab) -- VL(PSMA Fab | SAPFTFGPGTKVDIKRTVAAPSVFIFPPSDRKL | |
| EE/RK | KSGTASVVCLLNNFYPREAKVQWKVDNALQSGN | |
| | SQESVTEQDSKDSTYSLSSTLTLSKADYEKHKV | |
| | YACEVTHQGLSSPVTKSFNRGEC | |
| Ab-11 (Chain 3) | QVQLVESGGGVVQPGRSLSLSCAASGFAFSRYG | 722 |
| PSMA Fab LC: E123R; | MHWVRQAPGKGLEWVAVIWYDGSNKYYADSVKG | |
| Q124K | RFTISRDNSKNTQYLQMNSLRAEDTAVYYCARG | |
| PSMA Fab HC: R19S; | GDFLYYYYYGMDVWGQGTTVTVSSASTKGPSVF | |
| K153E; K219E | PLAPSSKSTSGGTAALGCLVEDYFPEPVTVSWN | |
| CD28 LC: CrossFab; SS | SGALTSGVHTFPAVLQSSGLYSLSSVVTVPSSS | |
| added | LGTQTYICNVNHKPSNTKVDEKVEPKSC | |
| CD28 HC: CrossFab; AS | | |
| replaced RT | | |
| linker connects CL(CD28 | | |
| crossFab) -- VL(PSMA Fab | | |
| EE/RK | | |
| Ab-12 (Chain 1) | QVQLVQSGAEVKKPGASVKVSCKASGYTFTSYY | 723 |
| PSMA Fab LC: E123R; | IHWVRQAPGQGLEWIGSIYPGNVNTNYNEKFKD | |
| Q124K | RATLTVDTSISTAYMELSRLRSDDTAVYFCTRS | |
| PSMA Fab HC: R19S; | HYGLDWNFDVWGQGTTVTVSSASVAAPSVFIFP | |
| K153E; K219E | PSDEQLKSGTASVVCLLNNFYPREAKVQWKVDN | |
| CD28 LC: CrossFab; SS | ALQSGNSQESVTEQDSKDSTYSLSSTLTLSKAD | |
| added | YEKHKVYACEVTHQGLSSPVTKSFNRGEC | |
| CD28 HC: CrossFab; AS | | |
| replaced RT; | | |
| linker connects CHI(CD28 | | |
| crossFab) -- VH(PSMA Fab | | |
| EE/RK) | | |
| Ab-12 (Chain 2) | DIQMTQSPSSLSASVGDRVTITCHASQNIYVWL | 724 |
| PSMA Fab LC: E123R; | NWYQQKPGKAPKLLIYKASNLHTGVPSRFSGSG | |
| Q124K | SGTDFTLTISSLQPEDFATYYCQQGQTYPYTFG | |
| PSMA Fab HC: R19S; | GGTKVEIKSSASTKGPSVFPLAPSSKSTSGGTA | |
| K153E; K219E | ALGCLVKDYFPEPVTVSWNSGALTSGVHTFPAV | |
| CD28 LC: CrossFab; SS | LQSSGLYSLSSVVTVPSSSLGTQTYICNVNHKP | |
| added | SNTKVDKKVEPKSCDGGGGSGGGGSQVQLVESG | |
| CD28 HC: CrossFab; AS | GGVVQPGRSLSLSCAASGFAFSRYGMHWVRQAP | |
| replaced RT; | GKGLEWVAVIWYDGSNKYYADSVKGRFTISRDN | |
| linker connects CHI(CD28 | SKNTQYLQMNSLRAEDTAVYYCARGGDFLYYYY | |
| crossFab) -- VH(PSMA Fab | YGMDVWGQGTTVTVSSASTKGPSVFPLAPSSKS | |
| EE/RK) | TSGGTAALGCLVEDYFPEPVTVSWNSGALTSGV | |
| | HTFPAVLQSSGLYSLSSVVTVPSSSLGTQTYIC | |
| | NVNHKPSNTKVDEKVEPKSC | |
| Ab-12 (Chain 3) | DIQMTQSPSSLSASVGDRVTITCRASQGISNYL | 725 |
| PSMA Fab LC: E123R; | AWYQQKTGKVPKFLIYEASTLQSGVPSRFSGGG | |
| Q124K | SGTDFTLTISSLQPEDVATYYCQNYNSAPFTFG | |
| PSMA Fab HC: R19S; | PGTKVDIKRTVAAPSVFIFPPSDRKLKSGTASV | |
| K153E; K219E | VCLLNNFYPREAKVQWKVDNALQSGNSQESVTE | |
| CD28 LC: CrossFab; SS | QDSKDSTYSLSSTLTLSKADYEKHKVYACEVTH | |
| added | QGLSSPVTKSFNRGEC | |
| CD28 HC: CrossFab; AS | | |
| replaced RT; | | |
| linker connects CHI(CD28 | | |
| crossFab) -- VH(PSMA Fab | | |
| EE/RK) | | |
| Ab-13 (Chain 1) | DIQMTQSPSSLSASVGDRVTITCRASQGISNYL | 726 |
| PSMA Fab LC: E123R; | AWYQQKTGKVPKFLIYEASTLQSGVPSRFSGGG | |
| Q124K | SGTDFTLTISSLQPEDVATYYCQNYNSAPFTFG | |
| PSMA Fab HC: R19S; | PGTKVDIKRTVAAPSVFIFPPSDRKLKSGTASV | |
| K153E; K219E | VCLLNNFYPREAKVQWKVDNALQSGNSQESVTE | |
| CD28 LC: CrossFab; SS | QDSKDSTYSLSSTLTLSKADYEKHKVYACEVTH | |
| added | QGLSSPVTKSFNRGECDGGGGSGGGGSQVQLVQ | |
| CD28 HC: CrossFab; AS | SGAEVKKPGASVKVSCKASGYTFTSYYIHWVRQ | |
| replaced RT | APGQGLEWIGSIYPGNVNTNYNEKFKDRATLTV | |
| linker connects CL(PSMA | DTSISTAYMELSRLRSDDTAVYFCTRSHYGLDW | |
| Fab EE/RK) -- VH(CD28 | NFDVWGQGTTVTVSSASVAAPSVFIFPPSDEQL | |
| CrossFab) | KSGTASVVCLLNNFYPREAKVQWKVDNALQSGN | |
| | SQESVTEQDSKDSTYSLSSTLTLSKADYEKHKV | |
| | YACEVTHQGLSSPVTKSFNRGEC | |
| Ab-13 (Chain 2) | QVQLVESGGGVVQPGRSLSLSCAASGFAFSRYG | 727 |
| PSMA Fab LC: E123R; | MHWVRQAPGKGLEWVAVIWYDGSNKYYADSVKG | |
| Q124K | RFTISRDNSKNTQYLQMNSLRAEDTAVYYCARG | |
| PSMA Fab HC: R19S; | GDFLYYYYYGMDVWGQGTTVTVSSASTKGPSVF | |
| K153E; K219E | PLAPSSKSTSGGTAALGCLVEDYFPEPVTVSWN | |
| CD28 LC: CrossFab; SS | SGALTSGVHTFPAVLQSSGLYSLSSVVTVPSSS | |
| added | LGTQTYICNVNHKPSNTKVDEKVEPKSC | |
| CD28 HC: CrossFab; AS | | |
| replaced RT | | |
| linker connects CL(PSMA | | |
| Fab EE/RK) -- VH(CD28 | | |
| CrossFab) | | |
| Ab-13 (Chain 3) | DIQMTQSPSSLSASVGDRVTITCHASQNIYVWLN | 728 |
| PSMA Fab LC: E123R: | WYQQKPGKAPKLLIYKASNLHTGVPSRFSGSGSG | |
| Q124K | TDFTLTISSLQPEDFATYYCQQGQTYPYTFGGGT | |
| PSMA Fab HC: R19S; | KVEIKSSASTKGPSVFPLAPSSKSTSGGTAALGC | |
| K153E; K219E | LVKDYFPEPVTVSWNSGALTSGVHTFPAVLQSSG | |
| CD28 LC: CrossFab; SS | LYSLSSVVTVPSSSLGTQTYICNVNHKPSNTKVD | |
| added | KKVEPKSC | |
| CD28 HC: CrossFab; AS | | |
| replaced RT | | |
| linker connects CL(PSMA | | |
| Fab EE/RK) -- VH(CD28 | | |
| CrossFab) | | |
| Ab-14 (Chain 1) | DIQMTQSPSSLSASVGDRVTITCRASQGISNYLA | 729 |
| PSMA Fab LC: E123R; | WYQQKTGKVPKFLIYEASTLQSGVPSRFSGGGSG | |
| Q124K | TDFTLTISSLQPEDVATYYCQNYNSAPFTFGPGT | |
| PSMA Fab HC: R19S; | KVDIKRTVAAPSVFIFPPSDRKLKSGTASVVCLL | |
| K153E; K219E | NNFYPREAKVQWKVDNALQSGNSQESVTEQDSKD | |
| CD28 LC: CrossFab; SS | STYSLSSTLTLSKADYEKHKVYACEVTHQGLSSP | |
| added | VTKSFNRGEC | |
| CD28 HC: CrossFab; AS | | |
| replaced RT | | |
| linker connects CHI(PSMA | | |
| Fab EE/RK) -- VL(CD28 | | |
| CrossFab) | | |
| Ab-14 (Chain 2) | QVQLVESGGGVVQPGRSLSLSCAASGFAFSRYGM | 730 |
| PSMA Fab LC: E123R: | HWVRQAPGKGLEWVAVIWYDGSNKYYADSVKGRF | |
| Q124K | TISRDNSKNTQYLQMNSLRAEDTAVYYCARGGDF | |
| PSMA Fab HC: R19S; | LYYYYYGMDVWGQGTTVTVSSASTKGPSVFPLAP | |
| K153E; K219E | SSKSTSGGTAALGCLVEDYFPEPVTVSWNSGALT | |
| CD28 LC: CrossFab; SS | SGVHTFPAVLQSSGLYSLSSVVTVPSSSLGTQTY | |
| added | ICNVNHKPSNTKVDEKVEPKSCDGGGGSGGGGSD | |
| CD28 HC: CrossFab; AS | IQMTQSPSSLSASVGDRVTITCHASQNIYVWLNW | |
| replaced RT | YQQKPGKAPKLLIYKASNLHTGVPSRFSGSGSGT | |
| linker connects CHI(PSMA | DFTLTISSLQPEDFATYYCQQGQTYPYTFGGGTK | |
| Fab EE/RK) -- VL(CD28 | VEIKSSASTKGPSVFPLAPSSKSTSGGTAALGCL | |
| CrossFab) | VKDYFPEPVTVSWNSGALTSGVHTFPAVLQSSGL | |
| | YSLSSVVTVPSSSLGTQTYICNVNHKPSNTKVDK | |
| | KVEPKSC | |
| Ab-14 (Chain 3) | QVQLVQSGAEVKKPGASVKVSCKASGYTFTSYYI | 731 |
| PSMA Fab LC: E123R; | HWVRQAPGQGLEWIGSIYPGNVNTNYNEKFKDRA | |
| Q124K | TLTVDTSISTAYMELSRLRSDDTAVYFCTRSHYG | |
| PSMA Fab HC: R19S; | LDWNFDVWGQGTTVTVSSASVAAPSVFIFPPSDE | |
| K153E; K219E | QLKSGTASVVCLLNNFYPREAKVQWKVDNALQSG | |
| CD28 LC: CrossFab; SS | NSQESVTEQDSKDSTYSLSSTLTLSKADYEKHKV | |
| added | YACEVTHQGLSSPVTKSFNRGEC | |
| CD28 HC: CrossFab; AS | | |
| replaced RT | | |
| linker connects CHI(PSMA | | |
| Fab EE/RK) -- VL(CD28 | | |
| CrossFab) | | |

In some embodiments, the isolated polypeptide or poly peptide complex is human or humanized. In some embodiments, the isolated polypeptide or polypeptide complex comprises a peptide that is linked to the anti-CD28 antibody, wherein the peptide impairs binding of the anti-CD28 antibody to CD28. In some embodiments, the isolated polypeptide or polypeptide complex comprises a configuration according to Formula I: A₂-A₁-L₁-P₁-H₁, wherein A₂ comprises the anti-PSMA antibody, A₁ comprises the anti-CD28 antibody, L₁ comprises a linking moiety that connects A₁ to P, and is a substrate for a tumor specific protease, P₁ comprises the peptide that impairs binding of the anti-CD28 antibody to CD28, and H₁ comprises a half-life extending molecule. In some embodiments, A₂ further comprises P₂ and L₂, wherein P₂ comprises a peptide that binds to A₂; and L₂ comprises a linking moiety that connects A₂ to P₂ and is a substrate for a tumor specific protease. In some embodiments, the polypeptide or polypeptide complex is according to Formula Ia: P₂-L₂-A₂-A₁-L₁-P₁-H₁.

In some embodiments, the anti-CD28 antibody comprises a scFv and the anti-PSMA antibody comprises a Fab or Fab′ and the Fab or Fab′ comprise a Fab heavy chain polypeptide and a Fab light chain polypeptide. In some embodiments, the Fab heavy chain polypeptide of A₂ is bound to the scFv heavy chain polypeptide of A₁ and L₂ is bound to the Fab light chain polypeptide of A₂. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

P₁ and P₂

In some embodiments, P₁ is bound to A₁ through ionic interactions, electrostatic interactions, hydrophobic interactions, Pi-stacking interactions, or H-bonding interactions, or a combination thereof. In some embodiments, P, has less than 70% sequence homology to CD28. In some embodiments, P₂ impairs binding of A₂ to PSMA. In some embodiments, P₂ is bound to A₂ through ionic interactions, electrostatic interactions, hydrophobic interactions, Pi-stacking interactions, or H-bonding interactions, or a combination thereof. In some embodiments, P₂ is bound to A₂ at or near an antigen binding site. In some embodiments, P₂ has less than 70% sequence homology to PSMA. In some embodiments, P₁ or P₂ comprises a peptide sequence of at least 10 amino acids in length. In some embodiments, P₁ or P₂ comprises a peptide sequence of at least 10 amino acids in length and no more than 20 amino acids in length. In some embodiments, P₁ or P₂ comprises a peptide sequence of at least 16 amino acids in length. In some embodiments, P₁ or P₂ comprises a peptide sequence of no more than 40 amino in length. In some embodiments, P₁ or P₂ comprises at least two cysteine amino acid residues. In some embodiments, P₁ or P₂ comprises a cyclic peptide or a linear peptide. In some embodiments, P₁ or P₂ comprises a cyclic peptide. In some embodiments, P₁ or P₂ comprises a linear peptide. In some embodiments, P₁ comprises at least two cysteine amino acid residues.

In some embodiments. P₁ comprises an amino acid sequence according to any one of SEQ ID NOs: 33 to 106, or 732-813. In some embodiments, P₁ comprises the amino acid sequence of SEQ ID NO: 33. In some embodiments, P₁ comprises the amino acid sequence of SEQ ID NO: 34. In some embodiments. P, comprises the amino acid sequence of SEQ ID NO: 35. In some embodiments, P₁ comprises the amino acid sequence of SEQ ID NO: 36. In some embodiments, P₁ comprises the amino acid sequence of SEQ ID NO: 37. In some embodiments, P₁ comprises the amino acid sequence of SEQ ID NO: 38. In some embodiments, P₁ comprises the amino acid sequence of SEQ ID NO: 39. In some embodiments, P, comprises the amino acid sequence of SEQ ID NO: 40. In some embodiments, P₁ comprises the amino acid sequence of SEQ ID NO: 41. In some embodiments. P₁ comprises the amino acid sequence of SEQ ID NO: 42. In some embodiments, P₁ comprises the amino acid sequence of SEQ ID NO: 43. In some embodiments, P₁ comprises the amino acid sequence of SEQ ID NO: 44. In some embodiments, P₁ comprises the amino acid sequence of SEQ ID NO: 45. In some embodiments, P₁ comprises the amino acid sequence of SEQ ID NO: 46. In some embodiments, P₁ comprises the amino acid sequence of SEQ ID NO: 47. In some embodiments, P₁ comprises the amino acid sequence of SEQ ID NO: 48. In some embodiments, P₁ comprises the amino acid sequence of SEQ ID NO: 49. In some embodiments, P₁ comprises the amino acid sequence of SEQ ID NO: 50. In some embodiments, P₁ comprises the amino acid sequence of SEQ ID NO: 51. In some embodiments, P₁ comprises the amino acid sequence of SEQ ID NO: 52. In some embodiments, P₁ comprises the amino acid sequence of SEQ ID NO: 53. In some embodiments, P₁ comprises the amino acid sequence of SEQ ID NO: 54. In some embodiments, P, comprises the amino acid sequence of SEQ ID NO: 55. In some embodiments, P₁ comprises the amino acid sequence of SEQ ID NO: 56. In some embodiments, P₁ comprises the amino acid sequence of SEQ ID NO: 57. In some embodiments, P₁ comprises the amino acid sequence of SEQ ID NO: 58. In some embodiments, P₁ comprises the amino acid sequence of SEQ ID NO: 59. In some embodiments, P, comprises the amino acid sequence of SEQ ID NO: 60.

In some embodiments, P₁ comprises the amino acid sequence of SEQ ID NO: 61. In some embodiments, P₁ comprises the amino acid sequence of SEQ ID NO: 62. In some embodiments, P, comprises the amino acid sequence of SEQ ID NO: 63. In some embodiments, P₁ comprises the amino acid sequence of SEQ ID NO: 64. In some embodiments, P₁ comprises the amino acid sequence of SEQ ID NO: 65. In some embodiments, P₁ comprises the amino acid sequence of SEQ ID NO: 66. In some embodiments, P₁ comprises the amino acid sequence of SEQ ID NO: 67. In some embodiments. P₁ comprises the amino acid sequence of SEQ ID NO: 68. In some embodiments, P₁ comprises the amino acid sequence of SEQ ID NO: 69. In some embodiments, P₁ comprises the amino acid sequence of SEQ ID NO: 70. In some embodiments, P₁ comprises the amino acid sequence of SEQ ID NO: 71. In some embodiments, P₁ comprises the amino acid sequence of SEQ ID NO: 72. In some embodiments, P, comprises the amino acid sequence of SEQ ID NO: 73. In some embodiments, P₁ comprises the amino acid sequence of SEQ ID NO: 74. In some embodiments, P₁ comprises the amino acid sequence of SEQ ID NO: 75. In some embodiments, P₁ comprises the amino acid sequence of SEQ ID NO: 76. In some embodiments, P₁ comprises the amino acid sequence of SEQ ID NO: 77. In some embodiments. P₁ comprises the amino acid sequence of SEQ ID NO: 78. In some embodiments, P₁ comprises the amino acid sequence of SEQ ID NO: 79. In some embodiments, P₁ comprises the amino acid sequence of SEQ ID NO: 80. In some embodiments, P₁ comprises the amino acid sequence of SEQ ID NO: 81. In some embodiments, P₁ comprises the amino acid sequence of SEQ ID NO: 82. In some embodiments, P, comprises the amino acid sequence of SEQ ID NO: 83. In some embodiments, P₁ comprises the amino acid sequence of SEQ ID NO: 84. In some embodiments, P₁ comprises the amino acid sequence of SEQ ID NO: 85. In some embodiments, P₁ comprises the amino acid sequence of SEQ ID NO: 86. In some embodiments, P₁ comprises the amino acid sequence of SEQ ID NO: 87. In some embodiments. P₁ comprises the amino acid sequence of SEQ ID NO: 88. In some embodiments, P₁ comprises the amino acid sequence of SEQ ID NO: 89. In some embodiments, P₁ comprises the amino acid sequence of SEQ ID NO: 90. In some embodiments, P₁ comprises the amino acid sequence of SEQ ID NO: 91. In some embodiments, P₁ comprises the amino acid sequence of SEQ ID NO: 92. In some embodiments, P₁ comprises the amino acid sequence of SEQ ID NO: 93. In some embodiments, P₁ comprises the amino acid sequence of SEQ ID NO: 94. In some embodiments, P₁ comprises the amino acid sequence of SEQ ID NO: 95. In some embodiments, P₁ comprises the amino acid sequence of SEQ ID N): 96. In some embodiments, P₁ comprises the amino acid sequence of SEQ ID NO: 97. In some embodiments, P, comprises the amino acid sequence of SEQ ID NO: 98. In some embodiments, P₁ comprises the amino acid sequence of SEQ ID NO: 99. In some embodiments, P₁ comprises the amino acid sequence of SEQ ID NO: 100. In some embodiments, P₁ comprises the amino acid sequence of SEQ ID NO: 101. In some embodiments, P₁ comprises the amino acid sequence of SEQ ID NO: 102. In some embodiments. P₁ comprises the amino acid sequence of SEQ ID NO: 103. In some embodiments, P₁ comprises the amino acid sequence of SEQ ID NO: 104. In some embodiments, P₁ comprises the amino acid sequence of SEQ ID NO: 105. In some embodiments, P₁ comprises the amino acid sequence of SEQ ID NO: 106.

TABLE 5
CD28 Peptide Mask Sequences

| Construct | Amino Acid Sequence | |
|---|---|---|
| Description | (N to C) | SEQ ID NO: |
| Peptide-1 | YWYCSPSIVRCVLV | 33 |
| Peptide-2 | LICKSGSILILCAQ | 34 |
| Peptide-3 | SPCGLFQWMEICEF | 35 |
| Peptide-4 | SFCGLFLDLWICEF | 36 |
| Peptide-5 | DNCYVIWGFEWQCR | 37 |
| Peptide-6 | VNCMRVHRTLTWCV | 38 |
| Peptide-7 | FCTPREWSFLNFVC | 39 |
| Peptide-8 | CFAYLWIDSWIRVC | 40 |
| Peptide-9 | MDWCPRERWVDCFF | 41 |
| Peptide-10 | GQHCATSMWRYCMF | 42 |
| Peptide-11 | WICDKSGSIMLCRA | 43 |
| Peptide-12 | GYCHYWGDMVMWCG | 44 |
| Peptide-13 | DNCHYIWGFEWQCG | 45 |
| Peptide-14 | IDCIMVHMVKPWCF | 46 |
| Peptide-15 | NCQPWYWNMFAFGC | 47 |
| Peptide-16 | NCQPWYWNMIAFGC | 48 |
| Peptide-17 | GCFTWSQRTFPFTC | 49 |
| Peptide-18 | CFYAEYYDQVYSFC | 50 |
| Peptide-19 | ADWCPRERWVDCFF | 51 |
| Peptide-20 | MAWCPRERWVDCFF | 52 |
| Peptide-21 | MDACPRERWVDCFF | 53 |
| Peptide-22 | MDWCARERWVDCFF | 54 |
| Peptide-23 | MDWCPAERWVDCFF | 55 |
| Peptide-24 | MDWCPRARWVDCFF | 56 |
| Peptide-25 | MDWCPREAWVDCFF | 57 |
| Peptide-26 | MDWCPRERAVDCFF | 58 |
| Peptide-27 | MDWCPRERWADCFF | 59 |
| Peptide-28 | MDWCPRERWVACFF | 60 |
| Peptide-29 | MDWCPRERWVDCAF | 61 |
| Peptide-30 | MDWCPRERWVDCFA | 62 |
| Peptide-31 | MDWCPIDLWNECFF | 63 |
| Peptide-32 | MDWCPIHLWHVCFN | 64 |
| Peptide-33 | MDWCPIYLWSECFN | 65 |
| Peptide-34 | MNWCPKDIWYLCFN | 66 |
| Peptide-35 | MDWCPLHMWHECFS | 67 |
| Peptide-36 | MDWCPLYLWNECFN | 68 |
| Peptide-37 | MDWCPRDLWDLCFA | 69 |
| Peptide-38 | MDWCPRDLWHECFA | 70 |
| Peptide-39 | MDWCPRDLWHLCFS | 71 |
| Peptide-40 | MDWCPRDLWSECFF | 72 |
| Peptide-41 | MDWCPRDLWVHCFA | 73 |
| Peptide-42 | MDWCPRDMWDECFA | 74 |
| Peptide-43 | MDWCPRDMWSECFA | 75 |
| Peptide-44 | MDWCPRDMWSVCFS | 76 |
| Peptide-45 | MDWCPRFMWDECFN | 77 |
| Peptide-46 | MDWCPRHMWNYCFA | 78 |
| Peptide-47 | MDWCPRSLWHECFA | 79 |
| Peptide-48 | MDWCPRYLWHVCFA | 80 |
| Peptide-49 | MHWCPVDLWYLCYN | 81 |
| Peptide-50 | MDWCPVHLWSVCFA | 82 |
| Peptide~52 | ADWCPRDLWVHCFA | 84 |
| Peptide-53 | RDWCPRDLWVHCFA | 85 |
| Peptide-54 | NDWCPRDLWVHCFA | 86 |
| Peptide-55 | DDWCPRDLWVHCFA | 87 |
| Peptide-56 | QDWCPRDLWVHCFA | 88 |
| Peptide-57 | EDWCPRDLWVHCFA | 89 |
| Peptide-58 | GDWCPRDLWVHCFA | 90 |
| Peptide-59 | HDWCPRDLWVHCFA | 91 |
| Peptide-60 | IDWCPRDLWVHCFA | 92 |
| Peptide-61 | LDWCPRDLWVHCFA | 93 |
| Peptide-62 | KDWCPRDLWVHCFA | 94 |
| Peptide-63 | FDWCPRDLWVHCFA | 95 |
| Peptide-64 | PDWCPRDLWVHCFA | 96 |
| Peptide-65 | SDWCPRDLWVHCFA | 97 |
| Peptide-66 | TDWCPRDLWVHCFA | 98 |
| Peptide-67 | WDWCPRDLWVHCFA | 99 |
| Peptide-68 | YDWCPRDLWVHCFA | 100 |
| Peptide-69 | VDWCPRDLWVHCFA | 101 |
| Peptide-70 | LDWCPRHLWALCFN | 102 |
| Peptide-71 | IDWCPRYLWDECYN | 103 |
| Peptide-72 | IDWCPLHLWHECYH | 104 |
| Peptide-73 | VPWCQIISGLECLT | 105 |
| Peptide-74 | IYWCPIDRWNDCYN | 106 |
| Peptide-75 | YWSCFQIPFTCFYF | 732 |
| Peptide-76 | WECLKYTEANACTY | 733 |
| Peptide-77 | FMCLPQNPHPLCFK | 734 |
| Peptide-78 | PDCQTILFKPWCVR | 735 |
| Peptide-79 | ACDTYFAGMINECF | 736 |
| Peptide-80 | GCQYPWAFDAMSCP | 737 |
| Peptide-81 | WCWDYKLWVPTQCP | 738 |
| Peptide-82 | WCWDYREWVPMTCP | 739 |
| Peptide-83 | ECHWSMWAVGHACF | 740 |
| Peptide-84 | YCAGHSPINIGKCL | 741 |
| Peptide-85 | WCWDYKLWVPMTCP | 742 |
| Peptide-86 | DCWWMFESYPGRCI | 743 |
| Peptide-87 | YCAGHAWQYINKCL | 744 |
| Peptide-88 | WCWYYREWVPMTCP | 745 |
| Peptide-89 | YWYCSPSIVRCVLV | 746 |
| Peptide-90 | PDCHTILFKPWCVR | 747 |
| Peptide-91 | WICDKSGSIMLCRA | 748 |
| Peptide-92 | SCAAGASYAWPYCL | 749 |
| Peptide-93 | DCPWLSGFSRPLLC | 750 |
| Peptide-94 | YCAGMSTIQLWLKC | 751 |
| Peptide-95 | YCAGHMPQYEVKCL | 752 |
| Peptide-96 | YCAGHTEEHYVKCM | 753 |
| Peptide-97 | YCMGHSHEHIWKCL | 754 |
| Peptide-98 | YCAGHSHEYLAKCE | 755 |
| Peptide-99 | ACAAGASYAWPYCL | 756 |
| Peptide-100 | SCAAAASYAWPYCL | 757 |
| Peptide-101 | SCAAGAAYAWPYCL | 758 |
| Peptide-102 | SCAAGASAAWPYCL | 759 |
| Peptide-103 | SCAAGASYAAPYCL | 760 |
| Peptide-104 | SCAAGASYAWAYCL | 761 |
| Peptide-105 | SCAAGASYAWPACL | 762 |
| Peptide-106 | SCAAGASYAWPYCA | 763 |
| Peptide-107 | ACAGHAWQYINKCL | 764 |
| Peptide-108 | YCAAHAWQYINKCL | 765 |
| Peptide-109 | YCAGAAWQYINKCL | 766 |
| Peptide-110 | YCAGHAAQYINKCL | 767 |
| Peptide-111 | YCAGHAWAYINKCL | 768 |
| Peptide-112 | YCAGHAWQAINKCL | 769 |
| Peptide-113 | YCAGHAWQYANKCL | 770 |
| Peptide-114 | YCAGHAWQYIAKCL | 771 |
| Peptide-115 | YCAGHAWQYINACL | 772 |
| Peptide-116 | YCAGHAWQYINKCA | 773 |
| Peptide-117 | YCEGHSPEYILKCL | 774 |
| Peptide-118 | YCEGHRPEYILKCL | 775 |
| Peptide-119 | YCEGHEGIYFWKCL | 776 |
| Peptide-120 | YCEGHLPEYIEKCL | 777 |
| Peptide-121 | YCEGHAPEYIAKCL | 778 |
| Peptide-122 | YCEGHAPEYIRKCL | 779 |
| Peptide-123 | YCEGHAPEYIWKCL | 780 |
| Peptide-124 | YCEGHSQENIWKCL | 781 |
| Peptide-125 | YCEGHLPEYLGKCL | 782 |
| Peptide-126 | YCEGHLPEYREKCL | 783 |
| Peptide-127 | YCEGHLPEYLAKCL | 784 |
| Peptide-128 | YCEGHSQEYLWKCL | 785 |
| Peptide-129 | YCEGHSSEYIQKCL | 786 |
| Peptide-130 | HCSGHHWLQVWKCF | 787 |
| Peptide-131 | YCWSHSPPFIKKCY | 788 |
| Peptide-132 | YCEGHLPEYRNKCL | 789 |
| Peptide-133 | YCEGHLPEYLEKCV | 790 |
| Peptide-134 | YCEGHLPEYIQKCL | 791 |
| Peptide-135 | YCEGHLPEYIWKCL | 792 |
| Peptide-136 | YCEGHAPEYIGKCL | 793 |
| Peptide-137 | TCSAGATWPWPICL | 794 |
| Peptide-138 | LCAAGASWPWPVCL | 795 |
| Peptide-139 | TCSAGASYPWPLCL | 796 |
| Peptide-140 | TCAAGAAWPWPQCL | 797 |
| Peptide-141 | TCSAGATYTWPKCF | 798 |
| Peptide-142 | TCAAGATWSWPACF | 799 |
| Peptide-143 | TCAAGAAWGWPLCF | 800 |
| Peptide-144 | LCAAGATYPWPLCL | 801 |
| Peptide-145 | TCSAGAAYPWPLCL | 802 |
| Peptide-146 | TCSAGAAYSWPACF | 803 |
| Peptide-147 | LCAAGATYPWPTCL | 804 |
| Peptide-148 | SCSAGAHYPWPQCF | 805 |
| Peptide-149 | TCSAGATYEWPRCL | 806 |
| Peptide-150 | TCSAGAHYPWPICL | 807 |
| Peptide-151 | SCSAGAAYYWPWCL | 808 |
| Peptide-152 | TCSAGATWAWPLCF | 809 |
| Peptide-153 | TCAAGATYLWPTCL | 810 |
| Peptide-154 | TCAAGAHYPWPACL | 811 |
| Peptide-155 | LCAAGFTYPWPLCL | 812 |
| Peptide-156 | LCKPGVPYKWPTCL | 813 |
| Peptide-288 | Met(O)-DWCPRDLWVHCFA | 4449 |
| Peptide-289 | Met(O)₂-DWCPRDLWVHCFA | 4450 |
| Peptide-290 | MDWCPRERWVDCFF | 4451 |

¹Met(O) is methionine sulfoxide; Met(O)₂ is methionine
sulfone.

In some embodiments, P₁ comprises the amino acid sequence of SEQ ID NO: 73 or a peptide sequence that has 1, 2, or 3 amino acid substitutions, additions, or deletions relative to the amino acid sequence of SEQ ID NO: 73. In some embodiments, P₁ comprises the amino acid sequence of SEQ ID NO: 73. In some embodiments, P₁ comprises the amino acid sequence of SEQ ID NO. 103 or a peptide sequence that has 1, 2, or 3 amino acid substitutions, additions, or deletions relative to the amino acid sequence of SEQ ID NO: 103. In some embodiments. P₁ comprises the amino acid sequence of SEQ ID NO: 103.

In some embodiments, P₁ comprises an amino acid sequence according to X₁-X₂-X₃-C-X₄-X₅-X₆-X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F: X₄ is selected from P, A, and L; X₈ is selected from R, T, I, M, S, K, L, V, W, F, A, P, and D, X₅, is selected from E, D, Y, H, S, F, A, N, R, S, Q, and H, X₈ is selected from Wand Q; X₉ is selected from H, N, D, A, S, Y, T, F, V, L, and I; X₁₀ is selected from E, V, L, D, Y, R, Q, H, F, K, A, M, and N; X₁₁ is selected from F, Y, LW, and V; and X₁₂ is selected from N, A, F, S, Y, H, D, T, and L. In some embodiments, X₁ is selected from M, I, and L; X₁ is selected from D, H, N, and A, X₃ is W; X₄ is P, X₈ is selected from R, E, D, Y, H, S, and F: X₇ is selected from L, M, and R, X₈ is W; X₉ is selected from H, N, D, A, S, and V; X₁₀ is selected from E, V, L, D, and H, X₁₁ is selected from F, Y, and L; and X₁₂ is selected from N, A, F, S, and Y. In some embodiments, X₁ is M; X₁ is selected from D and K, X₃ is W; X₄ is P; X₈ is selected from R, T, and I; X₆ is selected from E, D, and Y; X₇ is selected from L, M, and R; X₈ is W; X₉ is selected from H, N, D, and V; X₁₀ is selected from E, V, L, D, and H, X₁₁ is F; and X₁₂ is selected from N, A, and F.

In some embodiments, Pₘ comprises an amino acid sequence according to SEQ D NO: 41 or an amino acid sequence that has 1, 2, or 3 amino acid mutations, substitutions, or deletions relative to SEQ ID NO: 41. In some embodiments, P₁ comprises an amino acid sequence according to SEQ ID NO: 41. In some embodiments, P₁ comprises an amino acid sequence selected from any one of SEQ ID NOs: 41, 73, 103, and 179-603.

TABLE 6
Phage panning results of Anti-CD28 scFv Peptide-9 library sequences.
( ) indicates same amino acid as in anti-CD28 scFv
Peptide-9 corresponding position.
Peptide Sequences (P₁ or P₁ₐ)

| | | | | | | | | | | | | | | | | | | CD28 | |
|---|---|---|---|---|---|---|---|---|---|---|---|---|---|---|---|---|---|---|---|
| | Clonical | | | | | | | | | | | | | | | | | scFv | |
| | phage | Amino acid position sequence | | | | | | | | | | | | | | Back- | CD28 | signal in | SEQ |
| | peptide | | | | | | | | | | 1 | 1 | 1 | 1 | 1 | ground | scFv | presence | ID |
| ID | sequence | 1 | 2 | 3 | 4 | 5 | 6 | 7 | 8 | 9 | 0 | 1 | 2 | 3 | 4 | signal | signal | of CD28 | NO: |
| Phage- | MDWCPRER | M | D | W | C | P | R | E | R | W | V | D | C | F | F | 0.084 | 2.042 | 0.489 | 41 |
| 9 | WVDCFF | | | | | | | | | | | | | | | | | | |
| Phage- | MDWCPIDL | | | | | | I | D | L | | N | E | | | | 0.066 | 2.515 | 0.187 | 179 |
| 19 | WNECEF | | | | | | | | | | | | | | | | | | |
| Phage- | MDWCPIHL | | | | | | I | H | L | | H | V | | | N | 0.082 | 2.526 | 0.167 | 180 |
| 20 | WHVCFN | | | | | | | | | | | | | | | | | | |
| Phage- | MDWCPIYL | | | | | | I | Y | L | | S | E | | | N | 0,075 | 2.635 | 0.513 | 181 |
| 21 | WSECFN | | | | | | | | | | | | | | | | | | |
| Phage- | MNWCPKDI | | N | | | | K | D | I | | Y | L | | | N | 0.073 | 2.625 | 0.168 | 182 |
| 22 | WYLCFN | | | | | | | | | | | | | | | | | | |
| Phage- | MDWCPLHM | | | | | | L | H | M | | H | E | | | S | 0.086 | 2.511 | 0.151 | 183 |
| 23 | WHECFS | | | | | | | | | | | | | | | | | | |
| Phage- | MDWCPLYL | | | | | | L | Y | L | | N | E | | | N | 0.065 | 2.612 | 0.247 | 184 |
| 24 | WNECFN | | | | | | | | | | | | | | | | | | |
| Phage- | MDWCPRDL | | | | | | | D | L | | D | L | | | A | 0.078 | 2.696 | 0.219 | 185 |
| 25 | WDLCFA | | | | | | | | | | | | | | | | | | |
| Phage- | MDWCPRDL | | | | | | | D | L | | H | E | | | A | 0.063 | 2.710 | 0.277 | 186 |
| 26 | WHECFA | | | | | | | | | | | | | | | | | | |
| Phage- | MDWCPRDL | | | | | | | D | L | | H | L | | | S | 0.059 | 2.592 | 0.290 | 187 |
| 27 | WHLCFS | | | | | | | | | | | | | | | | | | |
| Phage- | MDWCPRDL | | | | | | | D | L | | S | E | | | | 0.068 | 2.574 | 0.218 | 188 |
| 28 | WSECFF | | | | | | | | | | | | | | | | | | |
| Phage- | MDWCPRDL | | | | | | | D | L | | | H | | | A | 0.062 | 2.554 | 0.179 | 73 |
| 29 | WVHCFA | | | | | | | | | | | | | | | | | | |
| Phage- | MDWCPRDM | | | | | | | D | M | | D | E | | | A | 0.116 | 2.593 | 0.250 | 189 |
| 30 | WDECFA | | | | | | | | | | | | | | | | | | |
| Phage- | MDWCPRDM | | | | | | | D | M | | S | E | | | A | 0.069 | 2.701 | 0.293 | 190 |
| 31 | WSECFA | | | | | | | | | | | | | | | | | | |
| Phage- | MDWCPRDM | | | | | | | D | M | | S | V | | | A | 0.062 | 2.619 | 0.207 | 191 |
| 32 | WSVCES | | | | | | | | | | | | | | | | | | |
| Phage- | MDWCPRFM | | | | | | | F | M | | D | E | | | N | 0.079 | 2.680 | 0.256 | 192 |
| 33 | WDECFN | | | | | | | | | | | | | | | | | | |
| Phage- | MDWCPRHM | | | | | | | H | M | | N | Y | | | A | 0.089 | 2.712 | 0.242 | 193 |
| 34 | WNYCFA | | | | | | | | | | | | | | | | | | |
| Phage- | MDWCPRSL | | | | | | | S | L | | H | E | | | A | 0.064 | 2.544 | 0.255 | 194 |
| 35 | WHECFA | | | | | | | | | | | | | | | | | | |
| Phage- | MDWCPRYL | | | | | | | Y | L | | H | V | | | A | 0.072 | 2.475 | 0.226 | 195 |
| 36 | WHVCFA | | | | | | | | | | | | | | | | | | |
| Phage- | MHWCPVDL | | H | | | | V | D | L | | Y | L | | Y | N | 0.079 | 2.659 | 0.417 | 196 |
| 37 | WYLCYN | | | | | | | | | | | | | | | | | | |
| Phage- | MDWCPVHL | | | | | | V | H | L | | S | V | | | A | 0.072 | 2.650 | 0.302 | 197 |
| 38 | WSVCFA | | | | | | | | | | | | | | | | | | |
| Phage- | MDWCPMHL | | | | | | M | H | L | | H | Q | | | N | 0.073 | 2.555 | 0.299 | 198 |
| 39 | WHQCFN | | | | | | | | | | | | | | | | | | |
| Phage- | MDWCPIDM | | | | | | I | D | M | | D | Q | | | N | 0.061 | 2.661 | 0.297 | 199 |
| 40 | WDQCFN | | | | | | | | | | | | | | | | | | |
| Phage- | MAWCPRDK | | A | | | | | D | K | | S | E | | | S | 0.062 | 0.853 | 0.147 | 200 |
| 41 | WSECFS | | | | | | | | | | | | | | | | | | |
| Phage- | MDWCPRHL | | | | | | | H | L | | | H | | | N | 0.071 | 2.505 | 0.254 | 201 |
| 42 | WVHCFN | | | | | | | | | | | | | | | | | | |
| Phage- | MDWCPRAI | | | | | | | A | L | | H | E | | | Y | 0.115 | 2.499 | 0.252 | 202 |
| 43 | WHECFY | | | | | | | | | | | | | | | | | | |
| Phage- | MDWCPIAL | | | | | | I | A | L | | A | E | | | N | 0.111 | 2.458 | 0.224 | 203 |
| 44 | WAECFN | | | | | | | | | | | | | | | | | | |
| Phage- | MDWCPRPL | | | | | | | P | L | | H | E | | | S | 0.078 | 2.346 | 0.121 | 204 |
| 45 | WHECFS | | | | | | | | | | | | | | | | | | |
| Phage- | MHWCPIDL | | H | | | | I | D | L | | A | E | | Y | A | 0.082 | 2.278 | 0.225 | 205 |
| 46 | WAECYA | | | | | | | | | | | | | | | | | | |
| Phage- | MAWCPVYL | | A | | | | V | Y | L | | H | E | | | N | 0.094 | 2.273 | 0.351 | 206 |
| 47 | WHECFN | | | | | | | | | | | | | | | | | | |
| Phage- | IDWCPRYL | I | | | | | | Y | L | | D | E | | Y | N | 0.061 | 2.243 | 0.153 | 103 |
| 48 | WDECYN | | | | | | | | | | | | | | | | | | |
| Phage- | MSWCPIHL | | S | | | | I | H | L | | N | E | | | N | 0.083 | 2.057 | 0.284 | 207 |
| 49 | WNECFN | | | | | | | | | | | | | | | | | | |
| Phage- | MDWCPPYL | | | | | | P | Y | L | | N | V | | | S | 0.063 | 1.639 | 0.100 | 208 |
| 50 | WNVCFS | | | | | | | | | | | | | | | | | | |
| Phage- | MDWCPMDL | | | | | | M | D | L | | D | Y | | | N | 0.071 | 1.635 | 0.107 | 209 |
| 51 | WDYCFN | | | | | | | | | | | | | | | | | | |
| Phage- | MDWCPINL | | | | | | I | N | L | | D | E | | | S | 0.062 | 1.498 | 0.096 | 210 |
| 52 | WDECFS | | | | | | | | | | | | | | | | | | |
| Phage- | MDWCPMHL | | | | | | M | H | L | | N | K | | | N | 0.071 | 1.043 | 0.122 | 211 |
| 53 | WNKCFN | | | | | | | | | | | | | | | | | | |
| Phage- | MNWCPRDM | | N | | | | | D | M | | Y | Q | | | N | 0.068 | 1.948 | 0.177 | 212 |
| 54 | WYQCFN | | | | | | | | | | | | | | | | | | |
| Phage- | QDWCPRHM | Q | | | | | | H | M | | S | F | | | H | 0.138 | 0.074 | 0.081 | 213 |
| 55 | WSFCFH | | | | | | | | | | | | | | | | | | |
| Phage- | MHWCPMDQ | | H | | | | M | D | Q | | S | N | | | N | 0.062 | 0.515 | 0.084 | 214 |
| 56 | WSNCFN | | | | | | | | | | | | | | | | | | |
| Phage- | MDWCPRHR | | | | | | | H | | | | | | | Y | 0.073 | 0.478 | 0.110 | 215 |
| 57 | WVDCFY | | | | | | | | | | | | | | | | | | |
| Phage- | MHWCPIDR | | H | | | | I | D | | | A | | | | N | 0.074 | 0.287 | 0.077 | 216 |
| 58 | WADCFN | | | | | | | | | | | | | | | | | | |
| Phage- | MDWCPRDS | | | | | | | D | S | | H | L | | | A | 0.073 | 2.446 | 0.147 | 217 |
| 59 | WHLCFA | | | | | | | | | | | | | | | | | | |
| Phage- | MDWCPRTL | | | | | | | T | L | | Y | L | | | N | 0.508 | 2.593 | 1.198 | 218 |
| 60 | WYLCFN | | | | | | | | | | | | | | | | | | |
| Phage- | MVSCPTTM | | V | S | | | T | T | M | | N | R | | | N | 0.079 | 1.329 | 0.072 | 219 |
| 61 | WNRCFN | | | | | | | | | | | | | | | | | | |
| Phage- | MDWCPSYL | | | | | | S | Y | L | | N | E | | | | 0.065 | 1.995 | 0.133 | 220 |
| 62 | WNECFF | | | | | | | | | | | | | | | | | | |
| Phage- | MDWCPRAL | | | | | | | A | L | | A | E | | | N | 0.068 | 2.676 | 0.339 | 221 |
| 64 | WAECFN | | | | | | | | | | | | | | | | | | |
| Phage- | MDWCPMYL | | | | | | M | Y | L | | N | E | | | N | 0.064 | 2.609 | 0.471 | 222 |
| 65 | WNECFN | | | | | | | | | | | | | | | | | | |
| Phage- | MDWCPRYL | | | | | | | Y | L | | N | E | | | Y | 0.079 | 2.537 | 0.358 | 223 |
| 66 | WNECFY | | | | | | | | | | | | | | | | | | |
| Phage- | MNWCPTAL | | N | | | | T | A | L | | H | V | | | N | 0.067 | 1.644 | 0.169 | 224 |
| 67 | WHVCFN | | | | | | | | | | | | | | | | | | |
| Phage- | MDWCPMYM | | | | | | M | Y | M | | Y | E | | | A | 0.080 | 2.479 | 0.358 | 225 |
| 68 | WYECFA | | | | | | | | | | | | | | | | | | |
| Phage- | MDWCPIHM | | | | | | I | H | M | | A | | | | A | 0.065 | 2.457 | 0.143 | 226 |
| 69 | WADCFA | | | | | | | | | | | | | | | | | | |
| Phage- | ITWCPSSM | I | T | | | | S | S | M | | N | R | | | I | 0.065 | 0.379 | 0.110 | 227 |
| 70 | WNRCFI | | | | | | | | | | | | | | | | | | |
| Phage- | MDWCPRYL | | | | | | | Y | L | | H | E | | | A | 0.071 | 2.628 | 0.429 | 228 |
| 71 | WHECFA | | | | | | | | | | | | | | | | | | |
| Phage- | MFWCPTTM | | F | | | | T | T | M | | N | R | | | T | 0.111 | 0.662 | 0.338 | 229 |
| 72 | WNRCFT | | | | | | | | | | | | | | | | | | |
| Phage- | MDWCPRAL | | | | | | | A | L | | H | E | | | N | 0.071 | 2.686 | 0.401 | 230 |
| 73 | WHECFN | | | | | | | | | | | | | | | | | | |
| Phage- | MDWCPRDM | | | | | | | D | M | | L | F | | Y | N | 0.097 | 2.707 | 1.023 | 231 |
| 74 | WLFCYN | | | | | | | | | | | | | | | | | | |
| Phage- | MDWCPRSH | | | | | | | S | H | | H | V | | Y | N | 0.072 | 1.951 | 0.281 | 232 |
| 75 | WHVCYN | | | | | | | | | | | | | | | | | | |
| Phage- | MDWCPRYL | | | | | | | Y | L | | S | | | | A | 0.084 | 2.727 | 1.530 | 233 |
| 76 | WSDCFA | | | | | | | | | | | | | | | | | | |
| Phage- | MDWCPFYL | | | | | | F | Y | L | | D | E | | | N | 0.092 | 2.672 | 0.291 | 234 |
| 77 | WDECFN | | | | | | | | | | | | | | | | | | |
| Phage- | MDWCPRHL | | | | | | | H | L | | H | E | | | N | 0.070 | 2.654 | 0.704 | 235 |
| 78 | WHECFN | | | | | | | | | | | | | | | | | | |
| Phage- | MDWCPRDL | | | | | | | D | L | | H | E | | | S | 0.066 | 2.649 | 0.382 | 236 |
| 79 | WHECFS | | | | | | | | | | | | | | | | | | |
| Phage- | MDWCPMYL | | | | | | M | Y | L | | N | E | | | S | 0.071 | 2.641 | 0.411 | 237 |
| 80 | WNECFS | | | | | | | | | | | | | | | | | | |
| Phage- | MDWCPREL | | | | | | | F | L | | S | V | | | N | 0.515 | 2.637 | 1.151 | 238 |
| 81 | WSVCFN | | | | | | | | | | | | | | | | | | |
| Phage- | MDWCPRDL | | | | | | | D | L | | T | E | | | A | 0.060 | 2.634 | 0.151 | 239 |
| 82 | WTECFA | | | | | | | | | | | | | | | | | | |
| Phage- | MDWCPREL | | | | | | | F | L | | D | E | | | N | 0.065 | 2.629 | 0.483 | 240 |
| 83 | WDECFN | | | | | | | | | | | | | | | | | | |
| Phage- | MDWCPKYL | | | | | | K | Y | L | | S | V | | | | 0.184 | 2.622 | 0.826 | 241 |
| 84 | WSVCFF | | | | | | | | | | | | | | | | | | |
| Phage- | MDWCPMDL | | | | | | M | D | L | | Y | Q | | | N | 0.063 | 2.617 | 0.187 | 242 |
| 85 | WYQCFN | | | | | | | | | | | | | | | | | | |
| Phage- | MDWCPRHL | | | | | | | H | L | | A | E | | | | 0.066 | 2.611 | 0.292 | 243 |
| 86 | WAECFF | | | | | | | | | | | | | | | | | | |
| Phage- | MDWCPKDL | | | | | | K | D | L | | Y | L | | | A | 0.200 | 2.608 | 0.213 | 244 |
| 87 | WYLCFA | | | | | | | | | | | | | | | | | | |
| Phage- | MDWCPIHL | | | | | | I | H | L | | H | Y | | | N | 0.078 | 2.602 | 0.453 | 245 |
| 88 | WHYCFN | | | | | | | | | | | | | | | | | | |
| Phage- | MDWCPRAL | | | | | | | A | L | | N | V | | | N | 0.065 | 2.598 | 0.472 | 246 |
| 89 | WNVCFN | | | | | | | | | | | | | | | | | | |
| Phage- | MDWCPIDL | | | | | | I | D | L | | H | L | | | Y | 0.062 | 2.590 | 0.488 | 247 |
| 90 | WHLCFY | | | | | | | | | | | | | | | | | | |
| Phage- | MDWCPRDL | | | | | | | D | L | | F | L | | Y | N | 0.060 | 2.587 | 0.424 | 248 |
| 91 | WELCYN | | | | | | | | | | | | | | | | | | |
| Phage- | MDWCPRHL | | | | | | | H | L | | H | E | | | | 0.364 | 2.583 | 0.373 | 249 |
| 92 | WHECFF | | | | | | | | | | | | | | | | | | |
| Phage- | MDWCPRYL | | | | | | | Y | L | | T | V | | | S | 0.115 | 2.582 | 0.372 | 250 |
| 93 | WTVCFS | | | | | | | | | | | | | | | | | | |
| Phage- | MDWCPRDL | | | | | | | D | L | | S | L | | | Y | 0.065 | 2.573 | 0.246 | 251 |
| 94 | WSLCFY | | | | | | | | | | | | | | | | | | |
| Phage- | MDWCPREL | | | | | | | F | L | | S | E | | | N | 0.062 | 2.571 | 0.227 | 252 |
| 95 | WSECFN | | | | | | | | | | | | | | | | | | |
| Phage- | MDWCPRTL | | | | | | | T | L | | A | Y | | | N | 0.070 | 2.571 | 0.584 | 253 |
| 96 | WAYCFN | | | | | | | | | | | | | | | | | | |
| Phage- | MDWCPKDL | | | | | | K | D | L | | D | Y | | | A | 0.078 | 2.568 | 0.165 | 254 |
| 97 | WDYCFA | | | | | | | | | | | | | | | | | | |
| Phage- | MDWCPKYL | | | | | | K | Y | L | | D | V | | | N | 0.096 | 2.566 | 0.422 | 255 |
| 98 | WDVCFN | | | | | | | | | | | | | | | | | | |
| Phage- | MDWCPRYL | | | | | | | Y | L | | N | M | | | H | 0.114 | 2.564 | 0.404 | 256 |
| 99 | WNMCFH | | | | | | | | | | | | | | | | | | |
| Phage- | MDWCPRYL | | | | | | | Y | L | | T | E | | | N | 0.062 | 2.560 | 0.549 | 257 |
| 100 | WTECFN | | | | | | | | | | | | | | | | | | |
| Phage- | MDWCPRSL | | | | | | | S | L | | H | Y | | | A | 0,140 | 2.552 | 0.338 | 258 |
| 101 | WHYCFA | | | | | | | | | | | | | | | | | | |
| Phage- | MDWCPRYL | | | | | | | Y | L | | A | E | | | Y | 0.063 | 2.552 | 0.354 | 259 |
| 102 | WAECFY | | | | | | | | | | | | | | | | | | |
| Phage- | MDWCPRDL | | | | | | | D | L | | H | E | | | N | 0.074 | 2.552 | 0.218 | 260 |
| 103 | WHECFN | | | | | | | | | | | | | | | | | | |
| Phage- | MDWCPRDL | | | | | | | D | L | | D | L | | | | 0.076 | 2.549 | 0.156 | 261 |
| 104 | WDLCFE | | | | | | | | | | | | | | | | | | |
| Phage- | MDWCPRYL | | | | | | | Y | L | | N | V | | | N | 0.087 | 2.546 | 0.570 | 262 |
| 105 | WNVCFN | | | | | | | | | | | | | | | | | | |
| Phage- | MDWCPIYL | | | | | | I | Y | L | | D | E | | | N | 0.061 | 2.546 | 0.215 | 263 |
| 106 | WDECFN | | | | | | | | | | | | | | | | | | |
| Phage- | MDWCPRDL | | | | | | | D | L | | A | E | | | N | 0.061 | 2.537 | 0.207 | 264 |
| 107 | WAECFN | | | | | | | | | | | | | | | | | | |
| Phage- | MDWCPRAL | | | | | | | A | L | | H | E | | | T | 0.075 | 2.536 | 0.191 | 265 |
| 108 | WHECFT | | | | | | | | | | | | | | | | | | |
| Phage- | MDWCPKNL | | | | | | K | N | L | | H | V | | | N | 0.081 | 2.530 | 0.248 | 266 |
| 109 | WHVCFN | | | | | | | | | | | | | | | | | | |
| Phage- | MDWCPRYL | | | | | | | Y | L | | D | E | | | N | 0.069 | 2.529 | 0.283 | 267 |
| 110 | WDECFN | | | | | | | | | | | | | | | | | | |
| Phage- | MDWCPFYL | | | | | | F | Y | L | | N | E | | | Y | 0.099 | 2.528 | 0.594 | 268 |
| 111 | WNECFY | | | | | | | | | | | | | | | | | | |
| Phage- | MHWCPRAL | | H | | | | | A | L | | D | V | | Y | N | 0.077 | 2.528 | 0.240 | 269 |
| 112 | WDVCYN | | | | | | | | | | | | | | | | | | |
| Phage- | MDWCPRDL | | | | | | | D | L | | N | V | | | | 0.164 | 2.520 | 0.137 | 270 |
| 113 | WNVCFF | | | | | | | | | | | | | | | | | | |
| Phage- | MDWCPRYL | | | | | | | Y | L | | F | E | | | A | 0.066 | 2.504 | 0.210 | 271 |
| 114 | WFECFA | | | | | | | | | | | | | | | | | | |
| Phage- | MDWCPRYL | | | | | | | Y | L | | H | E | | | N | 0.105 | 2.490 | 0.421 | 272 |
| 115 | WHECFN | | | | | | | | | | | | | | | | | | |
| Phage- | MDWCPRDL | | | | | | | D | L | | Y | A | | | A | 0.072 | 2.474 | 0.242 | 273 |
| 116 | WYACFA | | | | | | | | | | | | | | | | | | |
| Phage- | MDWCPRYL | | | | | | | Y | L | | F | E | | | S | 0.079 | 2.462 | 0.235 | 274 |
| 117 | WFECFS | | | | | | | | | | | | | | | | | | |
| Phage- | MDWCPRHL | | | | | | | H | L | | D | E | | | | 0.074 | 2.459 | 0.663 | 275 |
| 118 | WDECFF | | | | | | | | | | | | | | | | | | |
| Phage- | MDWCPRYL | | | | | | | Y | L | | H | M | | Y | S | 0.129 | 2.419 | 1.166 | 276 |
| 119 | WHMCYS | | | | | | | | | | | | | | | | | | |
| Phage- | MDWCPRDL | | | | | | | D | L | | H | A | | | S | 0.074 | 2.414 | 0.173 | 277 |
| 120 | WHACFS | | | | | | | | | | | | | | | | | | |
| Phage- | MDWCPRDL | | | | | | | D | L | | H | V | | | | 0.068 | 2.412 | 0.268 | 2.78 |
| 121 | WHVCFF | | | | | | | | | | | | | | | | | | |
| Phage- | MDWCPRDL | | | | | | | D | L | | D | Q | | Y | A | 0.059 | 2.406 | 0.201 | 279 |
| 122 | WDQCYA | | | | | | | | | | | | | | | | | | |
| Phage- | MDWCPIHL | | | | | | I | H | L | | N | E | | | A | 0.104 | 2.405 | 0.203 | 280 |
| 123 | WNECFA | | | | | | | | | | | | | | | | | | |
| Phage- | MDWCPRPL | | | | | | | P | L | | D | M | | | | 0.063 | 2.403 | 0.138 | 281 |
| 124 | WDMCFF | | | | | | | | | | | | | | | | | | |
| Phage- | MDWCPVSL | | | | | | V | S | L | | H | V | | | Y | 0.107 | 2.401 | 0.409 | 282 |
| 125 | WHVCFY | | | | | | | | | | | | | | | | | | |
| Phage- | MDWCPRFL | | | | | | | F | L | | N | E | | | N | 0.072 | 2.400 | 0.503 | 283 |
| 126 | WNECFN | | | | | | | | | | | | | | | | | | |
| Phage- | MDWCPRAL | | | | | | | A | L | | N | E | | | A | 0.080 | 2.385 | 0.375 | 284 |
| 127 | WNECFA | | | | | | | | | | | | | | | | | | |
| Phage- | MDWCPRDL | | | | | | | D | L | | I | E | | | | 0.084 | 2.372 | 0.137 | 285 |
| 128 | WIECFF | | | | | | | | | | | | | | | | | | |
| Phage- | MDWCPSYL | | | | | | S | Y | L | | T | V | | | A | 0.062 | 2.340 | 0.096 | 286 |
| 129 | WTVCFA | | | | | | | | | | | | | | | | | | |
| Phage- | MDWCPRYL | | | | | | | Y | L | | D | A | | | | 0.066 | 2.337 | 0.168 | 287 |
| 130 | WDACFF | | | | | | | | | | | | | | | | | | |
| Phage- | MDWCPRSL | | | | | | | S | L | | I | Y | | | N | 0.113 | 2.336 | 0.401 | 288 |
| 131 | WIYCFN | | | | | | | | | | | | | | | | | | |
| Phage- | MDWCPTYL | | | | | | T | Y | L | | F | E | | | N | 0.205 | 2.329 | 0.224 | 289 |
| 132 | WFECFN | | | | | | | | | | | | | | | | | | |
| Phage- | MDWCPREL | | | | | | | F | L | | D | E | | | | 0.064 | 2.284 | 0.246 | 290 |
| 133 | WDECFF | | | | | | | | | | | | | | | | | | |
| Phage- | MDWCPSYL | | | | | | S | Y | L | | H | E | | | A | 0.096 | 2.284 | 0.198 | 291 |
| 134 | WHECFA | | | | | | | | | | | | | | | | | | |
| Phage- | MDWCPKEL | | | | | | K | F | L | | H | E | | | S | 0.065 | 2.279 | 0.142 | 292 |
| 135 | WHECFS | | | | | | | | | | | | | | | | | | |
| Phage- | MHWCPIYL | | H | | | | I | Y | L | | D | E | | | N | 0.071 | 2.234 | 0.193 | 293 |
| 136 | WDECFN | | | | | | | | | | | | | | | | | | |
| Phage- | MDWCPRYL | | | | | | | Y | L | | H | E | | | H | 0.101 | 2.193 | 0.308 | 294 |
| 137 | WHECFH | | | | | | | | | | | | | | | | | | |
| Phage- | MDWCPTNL | | | | | | T | N | L | | H | E | | | A | 0.061 | 2.163 | 0.096 | 295 |
| 138 | WHECFA | | | | | | | | | | | | | | | | | | |
| Phage- | MDWCPRDL | | | | | | | D | L | | D | V | | | A | 0.096 | 2.158 | 0.280 | 296 |
| 139 | WDVCFA | | | | | | | | | | | | | | | | | | |
| Phage- | MDWCPMDL | | | | | | M | D | L | | D | V | | | N | 0.090 | 2.149 | 0.263 | 297 |
| 140 | WDVCFN | | | | | | | | | | | | | | | | | | |
| Phage- | MDWCPRSL | | | | | | | S | L | | N | V | | | | 0.106 | 2.131 | 0.356 | 298 |
| 141 | WNVCFF | | | | | | | | | | | | | | | | | | |
| Phage- | MDWCPVIL | | | | | | V | I | L | | D | F | | | N | 0.066 | 2.030 | 0.173 | 299 |
| 142 | WDECFN | | | | | | | | | | | | | | | | | | |
| Phage- | LDWCPLNL | L | | | | | L | N | L | | D | L | | Y | | 0.062 | 2.028 | 0.095 | 300 |
| 143 | WDLCYF | | | | | | | | | | | | | | | | | | |
| Phage- | MDWCPRHL | | | | | | | H | L | | Y | A | | | N | 0.061 | 2.012 | 0.159 | 301 |
| 144 | WYACFN | | | | | | | | | | | | | | | | | | |
| Phage- | MDWCPKHI | | | | | | K | H | L | | I | E | | | A | 0.065 | 1.977 | 0.132 | 302 |
| 145 | WIECFA | | | | | | | | | | | | | | | | | | |
| Phage- | MDWCPRHL | | | | | | | H | L | | S | E | | | Y | 0.136 | 1.918 | 0.199 | 303 |
| 146 | WSECFY | | | | | | | | | | | | | | | | | | |
| Phage- | MHWCPRDL | | H | | | | | D | L | | | V | | | N | 0.070 | 1.834 | 0.112 | 304 |
| 147 | WVVCFN | | | | | | | | | | | | | | | | | | |
| Phage- | MHWCPEYL | | H | | | | E | Y | L | | N | E | | | A | 0.074 | 1.687 | 0.092 | 305 |
| 148 | WNECFA | | | | | | | | | | | | | | | | | | |
| Phage- | MDWCPRDL | | | | | | | D | L | | A | V | | | A | 0.070 | 1.669 | 0.089 | 306 |
| 149 | WAVCFA | | | | | | | | | | | | | | | | | | |
| Phage- | MDWCPRHI | | | | | | | H | L | | N | V | | | S | 0.058 | 1.613 | 0.176 | 307 |
| 150 | WNVCFS | | | | | | | | | | | | | | | | | | |
| Phage- | MDFCPISL | | | F | | | I | S | L | | H | E | | | | 0.081 | 1.585 | 0.129 | 308 |
| 151 | WHECFF | | | | | | | | | | | | | | | | | | |
| Phage- | MDWCPKYL | | | | | | K | Y | L | | D | K | | | H | 0.073 | 1.540 | 0.109 | 309 |
| 152 | WDKCFH | | | | | | | | | | | | | | | | | | |
| Phage- | MDWCPRHL | | | | | | | H | L | | D | L | | | | 0.093 | 1.395 | 0.182 | 310 |
| 153 | WDLCFF | | | | | | | | | | | | | | | | | | |
| Phage- | MDWCPRDL | | | | | | | D | L | | N | V | | | A | 0.114 | 1.382 | 0.145 | 311 |
| 154 | WNVCFA | | | | | | | | | | | | | | | | | | |
| Phage- | MHWCPLHL | | H | | | | L | H | L | | N | E | | Y | H | 0.063 | 1.370 | 0.113 | 312 |
| 155 | WNECYH | | | | | | | | | | | | | | | | | | |
| Phage- | MDWCPKHI | | | | | | K | H | L | | H | Q | | | H | 0.058 | 1.351 | 0.108 | 313 |
| 156 | WH?CFH | | | | | | | | | | | | | | | | | | |
| Phage- | MDWCPRSL | | | | | | | S | L | | S | Y | | | H | 0.066 | 1.335 | 0.152 | 314 |
| 157 | WSYCFH | | | | | | | | | | | | | | | | | | |
| Phage- | MDWCPRYL | | | | | | | Y | L | | T | E | | | | 0.110 | 1.265 | 0.244 | 315 |
| 158 | WTECFE | | | | | | | | | | | | | | | | | | |
| Phage- | MAWCPMNL | | A | | | | M | N | L | | D | Q | | | | 0.070 | 1.202 | 0.145 | 316 |
| 159 | WDQCFF | | | | | | | | | | | | | | | | | | |
| Phage- | MHWCPRAL | | H | | | | | A | L | | H | E | | | N | 0.070 | 1.178 | 0.144 | 317 |
| 160 | WHECFN | | | | | | | | | | | | | | | | | | |
| Phage- | MDWCPRHL | | | | | | | H | L | | D | Q | | | A | 0.150 | 1.122 | 0.144 | 318 |
| 161 | WDQCFA | | | | | | | | | | | | | | | | | | |
| Phage- | MNWCPTDL | | N | | | | T | D | L | | H | E | | | N | 0.087 | 1.093 | 0.095 | 319 |
| 162 | WHECFN | | | | | | | | | | | | | | | | | | |
| Phage- | MEWCPRYL | | F | | | | | Y | L | | H | E | | | N | 0.086 | 1.078 | 0.175 | 320 |
| 163 | WHECFN | | | | | | | | | | | | | | | | | | |
| Phage- | MDWCPKEL | | | | | | K | F | L | | D | L | | | A | 0.100 | 1.075 | 0.139 | 321 |
| 164 | WDLCFA | | | | | | | | | | | | | | | | | | |
| Phage- | MDWCPFYL | | | | | | F | Y | L | | D | E | | | L | 0.070 | 1.024 | 0.141 | 322 |
| 165 | WDECFL | | | | | | | | | | | | | | | | | | |
| Phage- | MDWCPRHL | | | | | | | H | L | | D | L | | | A | 0.061 | 0.925 | 0.096 | 323 |
| 166 | WDLCFA | | | | | | | | | | | | | | | | | | |
| Phage- | MSWCPQDL | | S | | | | Q | D | L | | H | V | | | N | 0.095 | 0.860 | 0.112 | 324 |
| 167 | WHVCFN | | | | | | | | | | | | | | | | | | |
| Phage- | MDWCPKDL | | | | | | K | D | L | | H | E | | | N | 0.073 | 0.762 | 0.100 | 325 |
| 168 | WHECFN | | | | | | | | | | | | | | | | | | |
| Phage- | MDWCPRDL | | | | | | | D | L | | N | V | | | N | 0.100 | 0.740 | 0.108 | 326 |
| 169 | WNVCFN | | | | | | | | | | | | | | | | | | |
| Phage- | MNWCPSDL | | N | | | | S | D | L | | H | L | | | N | 0.071 | 0.739 | 0.131 | 327 |
| 170 | WHLCFN | | | | | | | | | | | | | | | | | | |
| Phage- | MNWCPSHL | | N | | | | S | H | L | | H | M | | Y | | 0.195 | 0.702 | 0.192 | 328 |
| 171 | WHMCYF | | | | | | | | | | | | | | | | | | |
| Phage- | MDWCPPYL | | | | | | P | Y | L | | Y | E | | | A | 0.068 | 0.692 | 0.094 | 329 |
| 172 | WYECFA | | | | | | | | | | | | | | | | | | |
| Phage- | MDWCPMNL | | | | | | M | N | L | | S | E | | | N | 0.168 | 0.670 | 0.124 | 330 |
| 173 | WSECFN | | | | | | | | | | | | | | | | | | |
| Phage- | MDWCPKHL | | | | | | K | H | L | | N | E | | | N | 0.063 | 0.663 | 0.105 | 331 |
| 174 | WNECFN | | | | | | | | | | | | | | | | | | |
| Phage- | MDWCPAYL | | | | | | A | Y | L | | A | E | | | S | 0.082 | 0.640 | 0.120 | 332 |
| 175 | WAECFS | | | | | | | | | | | | | | | | | | |
| Phage- | MDWCPSDL | | | | | | S | D | L | | H | E | | | H | 0.075 | 0.629 | 0.164 | 333 |
| 176 | WHECFH | | | | | | | | | | | | | | | | | | |
| Phage- | MDWCPVSL | | | | | | V | S | L | | D | H | | | N | 0.075 | 0.616 | 0.100 | 334 |
| 177 | WDHCFN | | | | | | | | | | | | | | | | | | |
| Phage- | LDWCPRDL | L | | | | | | D | L | | H | V | | | | 0.176 | 0.610 | 0.105 | 335 |
| 178 | WHVCFF | | | | | | | | | | | | | | | | | | |
| Phage- | MDWCPWIL | | | | | | W | I | L | | N | E | | | N | 0.141 | 0.581 | 0.137 | 336 |
| 179 | WNECFN | | | | | | | | | | | | | | | | | | |
| Phage- | MHWCPRYL | | H | | | | | Y | L | | D | E | | | N | 0.132 | 0.550 | 0.127 | 337 |
| 180 | WDECFN | | | | | | | | | | | | | | | | | | |
| Phage- | MYWCPRDL | | Y | | | | | D | L | | D | V | | | N | 0.145 | 0.512 | 0.102 | 338 |
| 181 | WDVCFN | | | | | | | | | | | | | | | | | | |
| Phage- | MHWCPRSI | | H | | | | | S | L | | N | E | | Y | | 0.168 | 0.456 | 0.115 | 339 |
| 182 | WNECYF | | | | | | | | | | | | | | | | | | |
| Phage- | IDWCPRDL | I | | | | | | D | L | | A | L | | | N | 0.127 | 0.370 | 0.129 | 340 |
| 183 | WALCFN | | | | | | | | | | | | | | | | | | |
| Phage- | MHWCPINL | | H | | | | I | N | L | | N | E | | | S | 0.081 | 0.316 | 0.098 | 341 |
| 184 | WNECFS | | | | | | | | | | | | | | | | | | |
| Phage- | MERCPREL | | E | R | | | | F | L | | N | E | | | N | 0.132 | 0.216 | 0.093 | 342 |
| 185 | WNECFN | | | | | | | | | | | | | | | | | | |
| Phage- | QDWCPTYL | Q | | | | | T | Y | L | | H | H | | | N | 0.101 | 0.123 | 0.122 | 343 |
| 186 | WHHCFN | | | | | | | | | | | | | | | | | | |
| Phage- | IGKLTLCL | I | G | K | L | T | L | C | L | N | A | | L | V | I | 0.274 | 0.116 | 0.100 | 344 |
| 187 | NADLVI | | | | | | | | | | | | | | | | | | |
| Phage- | MDWCPSYL | | | | | | S | Y | L | | D | Q | | | | 0.100 | 0.083 | 0.079 | 345 |
| 188 | WDQCFF | | | | | | | | | | | | | | | | | | |
| Phage- | VDWCPRYL | V | | | | | | Y | L | | H | V | | Y | N | 0.064 | 0.065 | 0.061 | 346 |
| 189 | WHVCYN | | | | | | | | | | | | | | | | | | |
| Phage- | MDWCPRDM | | | | | | | D | M | | Y | E | | | | 0.099 | 2.687 | 2.259 | 347 |
| 190 | WAECFF | | | | | | | | | | | | | | | | | | |
| Phage- | MDWCPRDM | | | | | | | D | M | | Y | E | | | N | 0.099 | 2.564 | 0.235 | 348 |
| 191 | WYECFN | | | | | | | | | | | | | | | | | | |
| Phage- | DVWCPKYM | D | V | | | | K | Y | M | | S | L | | | N | 0.101 | 2.529 | 0.418 | 349 |
| 192 | WSLCFN | | | | | | | | | | | | | | | | | | |
| Phage- | MDWCPMDM | | | | | | M | D | M | | | N | | | N | 0.059 | 2.516 | 0.277 | 350 |
| 193 | WVNCFN | | | | | | | | | | | | | | | | | | |
| Phage- | MDWCPSDM | | | | | | S | D | M | | H | E | | Y | A | 0.062 | 2.474 | 0.200 | 351 |
| 194 | WHECYA | | | | | | | | | | | | | | | | | | |
| Phage- | MDWCPKHM | | | | | | K | H | M | | F | M | | | N | 0.101 | 2.395 | 0.372 | 352 |
| 195 | WFMCFN | | | | | | | | | | | | | | | | | | |
| Phage- | MDWCPRYM | | | | | | | Y | M | | Y | Q | | | S | 0.079 | 2.364 | 0.357 | 353 |
| 196 | WYQCFS | | | | | | | | | | | | | | | | | | |
| Phage- | MDWCPRHM | | | | | | | H | M | | Y | E | | | | 0.129 | 2.342 | 0.298 | 354 |
| 197 | WYECFF | | | | | | | | | | | | | | | | | | |
| Phage- | MDWCPRAM | | | | | | | A | M | | N | H | | | N | 0.321 | 2.326 | 0.426 | 355 |
| 198 | WNHCFN | | | | | | | | | | | | | | | | | | |
| Phage- | MDWCPRNM | | | | | | | N | M | | A | Q | | | A | 0.102 | 2.315 | 0.209 | 356 |
| 199 | WAQCFA | | | | | | | | | | | | | | | | | | |
| Phage- | MFWCPFDM | | F | | | | F | D | M | | H | F | | | N | 0.150 | 2.292 | 0.469 | 357 |
| 200 | WHFCFN | | | | | | | | | | | | | | | | | | |
| Phage- | MDWCPRDM | | | | | | | D | M | | D | Q | | | D | 0.072 | 2.292 | 0.119 | 358 |
| 201 | WDQCFD | | | | | | | | | | | | | | | | | | |
| Phage- | MFWCPMDM | | F | | | | M | D | M | | D | Q | | | N | 0.091 | 2.260 | 0.207 | 359 |
| 202 | WDQCFN | | | | | | | | | | | | | | | | | | |
| Phage- | MSWCPRDM | | S | | | | | D | M | | F | Y | | Y | A | 0.065 | 2.248 | 0.174 | 360 |
| 203 | WFYCYA | | | | | | | | | | | | | | | | | | |
| Phage- | MDWCPRHM | | | | | | | H | M | | N | V | | | S | 0.067 | 2.245 | 0.130 | 361 |
| 204 | WNVCFS | | | | | | | | | | | | | | | | | | |
| Phage- | MDWCPTDM | | | | | | T | D | M | | H | H | | | L | 0.061 | 2.201 | 0.126 | 362 |
| 205 | WHHCFL | | | | | | | | | | | | | | | | | | |
| Phage- | IHWCPINM | I | H | | | | I | N | M | | D | K | | Y | N | 0.060 | 2.080 | 0.203 | 363 |
| 206 | WDKCYN | | | | | | | | | | | | | | | | | | |
| Phage- | MDWCPRAM | | | | | | | A | M | | H | E | | | Y | 0.069 | 1.915 | 0.129 | 364 |
| 207 | WHECFY | | | | | | | | | | | | | | | | | | |
| Phage- | MDWCPTDM | | | | | | T | D | M | | I | V | | | A | 0.363 | 1.495 | 0.172 | 365 |
| 208 | WIVCFA | | | | | | | | | | | | | | | | | | |
| Phage- | IDWCPQDM | I | | | | | Q | D | M | | F | Y | | | N | 0.069 | 1.416 | 0.160 | 366 |
| 209 | WFYCFN | | | | | | | | | | | | | | | | | | |
| Phage- | MDWCPRDM | | | | | | | D | M | | F | E | | | A | 0.076 | 1.310 | 0.143 | 367 |
| 210 | WFECFA | | | | | | | | | | | | | | | | | | |
| Phage- | MDWCPRNM | | | | | | | N | M | | T | V | | | L | 0.060 | 1.116 | 0.093 | 368 |
| 211 | WTVCFL | | | | | | | | | | | | | | | | | | |
| Phage- | MDWCPRAM | | | | | | | A | M | | D | K | | | | 0.073 | 0.985 | 0.094 | 369 |
| 212 | WDKCFF | | | | | | | | | | | | | | | | | | |
| Phage- | MNWCPSYM | | N | | | | S | Y | M | | D | Q | | | A | 0.125 | 0.854 | 0.105 | 370 |
| 213 | WDQCFA | | | | | | | | | | | | | | | | | | |
| Phage- | MDWCPTYM | | | | | | T | Y | M | | S | E | | | N | 0.086 | 0.754 | 0.107 | 371 |
| 214 | WSECFN | | | | | | | | | | | | | | | | | | |
| Phage- | MDWCPRYM | | | | | | | Y | M | | N | E | | | N | 0.064 | 0.687 | 0.128 | 372 |
| 215 | WNECFN | | | | | | | | | | | | | | | | | | |
| Phage- | MDWCPMNM | | | | | | M | N | M | | Y | Q | | | N | 0.110 | 0.639 | 0.137 | 373 |
| 216 | WYQCFN | | | | | | | | | | | | | | | | | | |
| Phage- | MDWCPWDM | | | | | | W | D | M | | D | K | | | N | 0.101 | 0.586 | 0.108 | 374 |
| 217 | WDKCFN | | | | | | | | | | | | | | | | | | |
| Phage- | TFGCPTTM | T | F | G | | | T | T | M | | N | R | | | A | 0.117 | 0.181 | 0.068 | 375 |
| 218 | WNRCFA | | | | | | | | | | | | | | | | | | |
| Phage- | NYWCPSSM | N | Y | | | | S | S | M | | N | R | | L | H | 0.295 | 0.158 | 0.088 | 376 |
| 219 | WNRCLH | | | | | | | | | | | | | | | | | | |
| Phage- | FDFCPTTM | F | | F | | | T | T | M | | T | Y | | Q | H | 0.084 | 0.086 | 0.081 | 377 |
| 220 | WTYCQH | | | | | | | | | | | | | | | | | | |
| Phage- | TTWCPTSM | T | T | | | | T | S | M | | L | H | | | D | 0.096 | 0.074 | 0.072 | 378 |
| 221 | WLHCFD | | | | | | | | | | | | | | | | | | |
| Phage- | MDWCPRDQ | | | | | | | D | Q | | H | N | | | N | 0.075 | 0.200 | 0.078 | 379 |
| 222 | WHNCFN | | | | | | | | | | | | | | | | | | |
| Phage- | MDWCPRDR | | | | | | | D | | | | | | | | 0.066 | 1.577 | 0.140 | 380 |
| 223 | WVDCFF | | | | | | | | | | | | | | | | | | |
| Phage- | MDWCPKDR | | | | | | K | D | | | N | | | Y | | 0.078 | 1.091 | 0.093 | 381 |
| 224 | WNDCYF | | | | | | | | | | | | | | | | | | |
| Phage- | MDWCPRDR | | | | | | | D | | | A | | | | | 0.069 | 0.392 | 0.089 | 382 |
| 225 | WADCFE | | | | | | | | | | | | | | | | | | |
| Phage- | MDWCPRDR | | | | | | | D | | | I | | | | N | 0.218 | 0.339 | 0.131 | 383 |
| 226 | WIDCFN | | | | | | | | | | | | | | | | | | |
| Phage- | MDWCPRDR | | | | | | | D | | | S | | | | N | 0.074 | 0.102 | 0.074 | 384 |
| 227 | WSDCFN | | | | | | | | | | | | | | | | | | |
| Phage- | ITWCHVIS | I | T | | | H | V | I | S | G | L | E | | W | N | 0.076 | 0.193 | 0.100 | 385 |
| 228 | GLECWN | | | | | | | | | | | | | | | | | | |
| Phage- | VPWCQIIS | V | P | | | Q | I | I | S | G | L | E | | L | T | 0.076 | 0.174 | 0.077 | 386 |
| 229 | GLECLT | | | | | | | | | | | | | | | | | | |
| Phage- | APWCQIIS | A | P | | | Q | I | I | S | G | L | E | | L | T | 0.080 | 0.169 | 0.078 | 387 |
| 230 | GLECLT | | | | | | | | | | | | | | | | | | |
| Phage- | VPWCLIIS | V | P | | | L | I | I | S | G | L | | | L | N | 0.455 | 0.107 | 0.090 | 388 |
| 231 | GLDCIN | | | | | | | | | | | | | | | | | | |
| Phage- | MDWCARFV | | | | | A | | F | V | G | Y | G | | L | D | 0.948 | 0.065 | 0.074 | 389 |
| 232 | GYGCLD | | | | | | | | | | | | | | | | | | |
| Phage- | MTWCPTSF | | T | | | | T | S | F | | N | R | | L | D | 0.142 | 0.392 | 0.405 | 390 |
| 233 | WNRCLD | | | | | | | | | | | | | | | | | | |
| Phage- | MDWCPRAL | | | | | | | A | L | | F | E | | | | 0.111 | 2.668 | 0.331 | 391 |
| 234 | WFECFF | | | | | | | | | | | | | | | | | | |
| Phage- | MDWCPRYL | | | | | | | Y | L | | H | E | | | S | 0.063 | 2.654 | 0.303 | 392 |
| 235 | WHECFS | | | | | | | | | | | | | | | | | | |
| Phage- | MDWCPRDL | | | | | | | D | L | | N | L | | | | 0.084 | 2.639 | 0.436 | 393 |
| 236 | WNLCFF | | | | | | | | | | | | | | | | | | |
| Phage- | MDWCPSYL | | | | | | S | Y | L | | H | E | | | | 0.067 | 2.606 | 0.447 | 394 |
| 237 | WHECFF | | | | | | | | | | | | | | | | | | |
| Phage- | MDWCPPYL | | | | | | P | Y | L | | S | E | | | A | 0.539 | 2.604 | 0.234 | 395 |
| 238 | WSECFA | | | | | | | | | | | | | | | | | | |
| Phage- | MDWCPRYL | | | | | | | Y | L | | H | V | | | N | 0.187 | 2.603 | 0.409 | 396 |
| 239 | WHVCFN | | | | | | | | | | | | | | | | | | |
| Phage- | MDWCPRTL | | | | | | | T | L | | H | V | | | N | 0.317 | 2.601 | 0.551 | 397 |
| 240 | WHVCFN | | | | | | | | | | | | | | | | | | |
| Phage- | MDWCPRHL | | | | | | | H | L | | H | E | | Y | S | 0.066 | 2.584 | 0.374 | 398 |
| 241 | WHECYS | | | | | | | | | | | | | | | | | | |
| Phage- | MDWCPKHI | | | | | | K | H | L | | T | E | | | A | 0.098 | 2.491 | 0.145 | 399 |
| 242 | WTECFA | | | | | | | | | | | | | | | | | | |
| Phage- | MDWCPRHL | | | | | | | H | L | | Y | E | | | N | 0.063 | 2.414 | 0.167 | 400 |
| 243 | WYECFN | | | | | | | | | | | | | | | | | | |
| Phage- | MDWCPRYL | | | | | | | Y | L | | H | E | | | D | 0.071 | 2.066 | 0.177 | 401 |
| 244 | WHECFD | | | | | | | | | | | | | | | | | | |
| Phage- | MDWCPRNL | | | | | | | N | L | | H | L | | | A | 0.082 | 1.397 | 0.164 | 402 |
| 245 | WHLCFA | | | | | | | | | | | | | | | | | | |
| Phage- | MDWCPKHL | | | | | | K | H | L | | N | K | | | N | 0.075 | 1.161 | 0.092 | 403 |
| 246 | WNKCFN | | | | | | | | | | | | | | | | | | |
| Phage- | MDWCPRFM | | | | | | | F | M | | A | E | | | N | 0.073 | 2.603 | 0.341 | 404 |
| 247 | WAECFN | | | | | | | | | | | | | | | | | | |
| Phage- | MDWCPRNM | | | | | | | N | M | | H | H | | | D | 0.205 | 1.597 | 0.524 | 405 |
| 248 | WHHCFD | | | | | | | | | | | | | | | | | | |
| Phage- | MDWCPSDM | | | | | | S | D | M | | A | H | | Y | N | 0.191 | 1.209 | 0.109 | 406 |
| 249 | WAHCYN | | | | | | | | | | | | | | | | | | |
| Phage- | MDWCPKVM | | | | | | K | V | M | | H | Y | | | A | 0.136 | 1.138 | 0.252 | 407 |
| 250 | WHYCFA | | | | | | | | | | | | | | | | | | |
| Phage- | MDWCPRFM | | | | | | | F | M | | S | E | | | S | 0.092 | 1.008 | 0.137 | 408 |
| 251 | WSECFS | | | | | | | | | | | | | | | | | | |
| Phage- | MDWCPRHM | | | | | | | H | M | | I | N | | | A | 0.091 | 0.920 | 0.165 | 409 |
| 252 | WINCFA | | | | | | | | | | | | | | | | | | |
| Phage- | MVGCATSM | | V | G | | A | T | S | M | | N | R | | L | T | 0.256 | 0.770 | 0.746 | 410 |
| 253 | WNRCLT | | | | | | | | | | | | | | | | | | |
| Phage- | MDWCPRYL | | | | | | | Y | L | | S | E | | | A | 0.078 | 2.705 | 0.513 | 411 |
| 254 | WSECFA | | | | | | | | | | | | | | | | | | |
| Phage- | MDWCPRHL | | | | | | | H | L | | S | V | | | N | 0.064 | 2.649 | 0.825 | 412 |
| 255 | WSVCFN | | | | | | | | | | | | | | | | | | |
| Phage- | MDWCPFDL | | | | | | F | D | L | | H | V | | Y | N | 0.089 | 2.602 | 0,651 | 413 |
| 256 | WHVCYN | | | | | | | | | | | | | | | | | | |
| Phage- | MDWCPRFL | | | | | | | F | L | | H | E | | | N | 0.079 | 2.586 | 0.351 | 414 |
| 257 | WHECFN | | | | | | | | | | | | | | | | | | |
| Phage- | MDWCPRSI | | | | | | | S | L | | H | V | | | N | 0.098 | 2.412 | 0.238 | 415 |
| 258 | WHVCFN | | | | | | | | | | | | | | | | | | |
| Phage- | MDWCPRNL | | | | | | | N | L | | H | A | | | N | 0.071 | 1.571 | 0.112 | 416 |
| 259 | WHACFN | | | | | | | | | | | | | | | | | | |
| Phage- | MDWCPVEM | | | | | | V | F | M | | N | E | | | N | 0.079 | 2.661 | 0.356 | 417 |
| 260 | WNECFN | | | | | | | | | | | | | | | | | | |
| Phage- | MDWCPMFM | | | | | | M | F | M | | H | E | | | N | 0.102 | 2.235 | 1.045 | 418 |
| 261 | WHECFN | | | | | | | | | | | | | | | | | | |
| Phage- | MYWCATSM | | Y | | | A | T | S | M | | N | R | | | V | 0.248 | 0.398 | 0.415 | 419 |
| 262 | WNRCFV | | | | | | | | | | | | | | | | | | |
| Phage- | MDWCPRHM | | | | | | | H | M | | H | E | | | A | 0.074 | 2.518 | 0.298 | 420 |
| 263 | WHECFA | | | | | | | | | | | | | | | | | | |
| Phage- | MDWCPRHL | | | | | | | H | L | | H | V | | | N | 0.070 | 2.667 | 0.636 | - |
| 264 | WHVCFN | | | | | | | | | | | | | | | | | | 421 |
| Phage- | MDWCPRFM | | | | | | | F | M | | D | L | | | A | 0.270 | 2.638 | 0.725 | 422 |
| 265 | WDLCFA | | | | | | | | | | | | | | | | | | |
| Phage- | VTSCPTTM | V | T | S | | | T | T | M | | N | R | | | S | 0.396 | 1.220 | 0.546 | 423 |
| 266 | WNRCFS | | | | | | | | | | | | | | | | | | |
| Phage- | MTLCLSVD | | T | L | | L | S | V | D | L | | H | | W | Y | 0.114 | 0.632 | 0.093 | 424 |
| 267 | LVHCWY | | | | | | | | | | | | | | | | | | |
| Phage- | DSFCTWSA | D | S | F | | T | W | S | A | | Q | E | | G | R | 0.072 | 0.130 | 0.101 | 425 |
| 269 | WQECGR | | | | | | | | | | | | | | | | | | |
| Phage- | MDWCPRDF | | | | | | | D | F | | A | F | | | | 0.118 | 2.365 | 0.655 | 426 |
| 270 | WAFCFF | | | | | | | | | | | | | | | | | | |
| Phage- | MDWCPTSE | | | | | | T | S | F | | N | R | | | H | 0.102 | 0.087 | 0.092 | 427 |
| 271 | WNRCFH | | | | | | | | | | | | | | | | | | |
| Phage- | MVWCMSVG | | V | | | M | S | V | G | | A | V | | L | N | 0.289 | 0.271 | 0.284 | 428 |
| 272 | WAVCLN | | | | | | | | | | | | | | | | | | |
| Phage- | IDWCPTAG | I | | | | | T | A | G | | T | Y | | W | | 0.071 | 0.136 | 0.075 | 429 |
| 273 | WTYCWF | | | | | | | | | | | | | | | | | | |
| Phage- | WVSCLRHH | W | V | S | | L | | H | H | | L | E | | | H | 0.781 | 0.723 | 0.858 | 430 |
| 274 | WLECFH | | | | | | | | | | | | | | | | | | |
| Phage- | MDWCPRDH | | | | | | | D | H | | | | | Y | S | 0.172 | 0.671 | 0.092 | 431 |
| 275 | WVDCYS | | | | | | | | | | | | | | | | | | |
| Phage- | IDWCPKIH | I | | | | | K | I | H | | D | L | | Y | | 0.072 | 0.295 | 0.082 | 432 |
| 276 | WDLCYF | | | | | | | | | | | | | | | | | | |
| Phage- | MDWCPRSH | | | | | | | S | H | | S | E | | | S | 0.080 | 0.253 | 0.087 | 433 |
| 277 | WSECFS | | | | | | | | | | | | | | | | | | |
| Phage- | MDWCPRSI | | | | | | | S | I | | Y | L | | | N | 0.383 | 2.548 | 1.337 | 434 |
| 278 | WYLCFN | | | | | | | | | | | | | | | | | | |
| Phage- | MDWCPRYL | | | | | | | Y | L | | A | E | | | N | 0.068 | 2.742 | 1.141 | 435 |
| 279 | WAECFN | | | | | | | | | | | | | | | | | | |
| Phage- | MDWCPRYL | | | | | | | Y | L | | D | Y | | | A | 0.073 | 2.709 | 0.382 | 436 |
| 280 | WDYCFA | | | | | | | | | | | | | | | | | | |
| Phage- | MDWCPRSL | | | | | | | S | L | | F | L | | | N | 1.110 | 2.709 | 1.774 | 437 |
| 281 | WFLCFN | | | | | | | | | | | | | | | | | | |
| Phage- | MDWCPRDL | | | | | | | D | L | | F | A | | | A | 0.060 | 2.658 | 0.794 | 438 |
| 282 | WFACFA | | | | | | | | | | | | | | | | | | |
| Phage- | MDWCPRHL | | | | | | | H | L | | N | E | | | N | 0.079 | 2.650 | 0.393 | 439 |
| 283 | WNECFN | | | | | | | | | | | | | | | | | | |
| Phage- | MDWCPREL | | | | | | | F | L | | A | M | | | A | 2.091 | 2.643 | 2,480 | 440 |
| 284 | WHVCFS | | | | | | | | | | | | | | | | | | |
| Phage- | MDWCPRDL | | | | | | | D | L | | A | M | | | A | 0.071 | 2.627 | 0.889 | 441 |
| 285 | WAMCFA | | | | | | | | | | | | | | | | | | |
| Phage- | MERCPRYL | | E | R | | | | Y | L | | H | M | | Y | S | 0.164 | 2.589 | 0.840 | 442 |
| 286 | WHMCYS | | | | | | | | | | | | | | | | | | |
| Phage- | MDWCPIAL | | | | | | I | A | L | | D | F | | | A | 0.073 | 2.522 | 0.492 | 443 |
| 288 | WDFCFA | | | | | | | | | | | | | | | | | | |
| Phage- | MDWCPRYL | | | | | | | Y | L | | N | V | | | A | 0.070 | 2.513 | 0.280 | 444 |
| 289 | WNVCFA | | | | | | | | | | | | | | | | | | |
| Phage- | MDWCPRDL | | | | | | | D | L | | S | V | | | S | 0.090 | 2.505 | 0.116 | 445 |
| 290 | WSVCFS | | | | | | | | | | | | | | | | | | |
| Phage- | MDWCPRYL | | | | | | | Y | L | | H | V | | | Y | 0.227 | 2.498 | 1.137 | 446 |
| 291 | WHVCFY | | | | | | | | | | | | | | | | | | |
| Phage- | MDWCPRAL | | | | | | | A | L | | D | H | | | | 0.081 | 2.492 | 0.394 | 447 |
| 292 | WDHCFF | | | | | | | | | | | | | | | | | | |
| Phage- | MDWCPRHI | | | | | | | H | L | | F | E | | | N | 0.064 | 2.489 | 0.171 | 448 |
| 293 | WFECFN | | | | | | | | | | | | | | | | | | |
| Phage- | MDWCPRSL | | | | | | | S | L | | D | Y | | | A | 0.115 | 2.488 | 0.235 | 449 |
| 294 | WDYCFA | | | | | | | | | | | | | | | | | | |
| Phage- | MDWCPRHL | | | | | | | H | L | | T | E | | | S | 0.074 | 2.482 | 0.243 | 450 |
| 297 | WTECFS | | | | | | | | | | | | | | | | | | |
| Phage- | MDWCPLYL | | | | | | L | Y | L | | A | E | | | N | 0.094 | 2.476 | 0.193 | 451 |
| 298 | WAECFN | | | | | | | | | | | | | | | | | | |
| Phage- | MDWCPIYL | | | | | | I | Y | L | | A | E | | | N | 0.078 | 2.453 | 0.307 | 452 |
| 299 | WAECFN | | | | | | | | | | | | | | | | | | |
| Phage- | MDWCPRHL | | | | | | | H | L | | F | E | | | | 0.178 | 2.451 | 0.479 | 453 |
| 300 | WFECFF | | | | | | | | | | | | | | | | | | |
| Phage- | MDWCPRYL | | | | | | | Y | L | | H | E | | | | 0.082 | 2.450 | 0.682 | 454 |
| 301 | WHECFF | | | | | | | | | | | | | | | | | | |
| Phage- | MDWCPRYL | | | | | | | Y | L | | H | V | | | D | 0.068 | 2.445 | 0.310 | 455 |
| 302 | WHVCFD | | | | | | | | | | | | | | | | | | |
| Phage- | MDWCPRYL | | | | | | | Y | L | | T | E | | | S | 0.074 | 2.421 | 0.329 | 456 |
| 303 | WTECFS | | | | | | | | | | | | | | | | | | |
| Phage- | MDWCPKEL | | | | | | K | F | L | | D | E | | | A | 0.311 | 2.406 | 0.376 | 457 |
| 304 | WDECFA | | | | | | | | | | | | | | | | | | |
| Phage- | MDWCPRDL | | | | | | | D | L | | T | E | | | S | 0.080 | 2.401 | 0.174 | 458 |
| 305 | WTECFS | | | | | | | | | | | | | | | | | | |
| Phage- | MDWCPRHL | | | | | | | H | L | | N | E | | | A | 0.068 | 2.394 | 0.170 | 459 |
| 306 | WNECFA | | | | | | | | | | | | | | | | | | |
| Phage- | MDWCPRYL | | | | | | | Y | L | | P | V | | | H | 0.179 | 2.392 | 0.602 | 460 |
| 307 | WPVCFH | | | | | | | | | | | | | | | | | | |
| Phage- | MDWCPKSL | | | | | | K | S | L | | A | E | | | N | 0.395 | 2.377 | 0.162 | 461 |
| 308 | WAECFN | | | | | | | | | | | | | | | | | | |
| Phage- | MDWCPMFL | | | | | | M | F | L | | H | E | | | N | 0.170 | 2.375 | 0.591 | 462 |
| 309 | WHECFN | | | | | | | | | | | | | | | | | | |
| Phage- | MDWCPRDL | | | | | | | D | L | | D | E | | | N | 0.066 | 2.374 | 0.248 | 463 |
| 310 | WDECFN | | | | | | | | | | | | | | | | | | |
| Phage- | MDWCPRDL | | | | | | | D | L | | Y | Q | | | N | 0.069 | 2.353 | 0.609 | 464 |
| 311 | WYQCFN | | | | | | | | | | | | | | | | | | |
| Phage- | MDWCPRSL | | | | | | | S | L | | N | Y | | | N | 0.076 | 2.350 | 0.209 | 465 |
| 313 | WNYCFN | | | | | | | | | | | | | | | | | | |
| Phage- | MDWCPIHL | | | | | | I | H | L | | N | E | | | N | 0.074 | 2.347 | 0.157 | 466 |
| 314 | WNECFN | | | | | | | | | | | | | | | | | | |
| Phage- | MDWCPTYL | | | | | | T | Y | L | | H | V | | | S | 0.089 | 2.324 | 0.160 | - |
| 315 | WHVCFS | | | | | | | | | | | | | | | | | | 467 |
| Phage- | MDWCPRSL | | | | | | | S | L | | N | Y | | | A | 0.067 | 2.316 | 0.198 | 468 |
| 316 | WNYCFA | | | | | | | | | | | | | | | | | | |
| Phage- | MDWCPRHL | | | | | | | H | L | | H | E | | | T | 0.103 | 2.297 | 0.320 | 469 |
| 317 | WHECFT | | | | | | | | | | | | | | | | | | |
| Phage- | MDWCPMEL | | | | | | M | F | L | | D | E | | | N | 0.132 | 2.287 | 0.203 | 470 |
| 318 | WDECFN | | | | | | | | | | | | | | | | | | |
| Phage- | MSWCPRDL | | S | | | | | D | L | | H | L | | | | 0.077 | 2.265 | 0.319 | 471 |
| 319 | WHLCFF | | | | | | | | | | | | | | | | | | |
| Phage- | MDWCPTYL | | | | | | T | Y | L | | N | E | | | N | 0.120 | 2.244 | 0.606 | 472 |
| 320 | WNECFN | | | | | | | | | | | | | | | | | | |
| Phage- | MDWCPRDL | | | | | | | D | L | | Y | V | | | A | 0.095 | 2.221 | 0.138 | 473 |
| 321 | WYVCFA | | | | | | | | | | | | | | | | | | |
| Phage- | MDWCPRHL | | | | | | | H | L | | I | E | | | N | 0.339 | 2.214 | 0.376 | 474 |
| 322 | WIECFN | | | | | | | | | | | | | | | | | | |
| Phage- | MDWCPTYL | | | | | | T | Y | L | | N | V | | | A | 0.085 | 2.211 | 0.132 | 475 |
| 323 | WNVCFA | | | | | | | | | | | | | | | | | | |
| Phage- | MDWCPRHL | | | | | | | H | L | | Y | E | | | S | 0.216 | 2.167 | 0.163 | 476 |
| 324 | WYECFS | | | | | | | | | | | | | | | | | | |
| Phage- | MDWCPAYL | | | | | | A | Y | L | | D | E | | | A | 0.096 | 2.166 | 0.163 | 477 |
| 325 | WDECFA | | | | | | | | | | | | | | | | | | |
| Phage- | MFWCPITL | | F | | | | I | T | L | | N | E | | | N | 0.075 | 2.153 | 0.143 | 478 |
| 326 | WNECFN | | | | | | | | | | | | | | | | | | |
| Phage- | MHWCPRDL | | H | | | | | D | L | | H | V | | | N | 0.071 | 2.149 | 0.150 | 479 |
| 327 | WHVCFN | | | | | | | | | | | | | | | | | | |
| Phage- | MDWCPRAL | | | | | | | A | L | | D | H | | | N | 0.323 | 2.145 | 0.293 | 480 |
| 328 | WDHCFN | | | | | | | | | | | | | | | | | | |
| Phage- | MDWCPRAL | | | | | | | A | L | | N | V | | | N | 0.071 | 2.140 | 0.249 | 481 |
| 329 | WNVCFA | | | | | | | | | | | | | | | | | | |
| Phage- | MDWCPREL | | | | | | | F | L | | D | V | | | N | 0.100 | 2.139 | 0.309 | 482 |
| 330 | WDVCFN | | | | | | | | | | | | | | | | | | |
| Phage- | MDWCPRHL | | | | | | | H | L | | D | E | | | Y | 0.098 | 2.127 | 0.146 | 483 |
| 331 | WDECFY | | | | | | | | | | | | | | | | | | |
| Phage- | IDWCPRAL | I | | | | | | A | L | | D | A | | L | A | 0.269 | 2.103 | 0.164 | 484 |
| 332 | WDACLA | | | | | | | | | | | | | | | | | | |
| Phage- | MDWCPTDL | | | | | | T | D | L | | H | E | | | A | 0.078 | 2.071 | 0.154 | 485 |
| 333 | WHECFA | | | | | | | | | | | | | | | | | | |
| Phage- | MDWCPRYL | | | | | | | Y | L | | D | E | | | S | 0.069 | 2.064 | 0.164 | 486 |
| 334 | WDECFS | | | | | | | | | | | | | | | | | | |
| Phage- | MDWCPRFL | | | | | | | F | L | | D | Y | | | A | 0.146 | 2.034 | 0.207 | 487 |
| 335 | WDYCFA | | | | | | | | | | | | | | | | | | |
| Phage- | MDWCPRDL | | | | | | | D | L | | N | W | | | N | 0.139 | 2.026 | 0.390 | 488 |
| 336 | WNWCFN | | | | | | | | | | | | | | | | | | |
| Phage- | MDWCPKPL | | | | | | K | P | L | | H | V | | | A | 0.069 | 2.022 | 0.152 | 489 |
| 337 | WHVCFA | | | | | | | | | | | | | | | | | | |
| Phage- | MDWCPRFL | | | | | | | F | L | | N | E | | | Y | 0.143 | 1.996 | 0.287 | 490 |
| 338 | WNECFY | | | | | | | | | | | | | | | | | | |
| Phage- | MDWCPRTL | | | | | | | T | L | | D | Q | | | | 0.088 | 1.992 | 0.314 | 491 |
| 339 | WDQCFF | | | | | | | | | | | | | | | | | | |
| Phage- | MSWCPIDL | | S | | | | I | D | L | | S | E | | | A | 0.072 | 1.986 | 0.148 | 492 |
| 340 | WSECFA | | | | | | | | | | | | | | | | | | |
| Phage- | MDWCPRHL | | | | | | | H | L | | D | E | | | N | 0.084 | 1.978 | 0.268 | 493 |
| 341 | WDECFN | | | | | | | | | | | | | | | | | | |
| Phage- | MDWCPRNL | | | | | | | N | L | | H | E | | | A | 0.067 | 1.965 | 0.175 | 494 |
| 342 | WHECFA | | | | | | | | | | | | | | | | | | |
| Phage- | MDWCPRDL | | | | | | | D | L | | T | Q | | | | 0.065 | 1.932 | 0.119 | 495 |
| 343 | WTQCFF | | | | | | | | | | | | | | | | | | |
| Phage- | MDWCPRHL | | | | | | | H | L | | | H | | | S | 0.096 | 1.914 | 0.173 | 496 |
| 344 | WVHCFS | | | | | | | | | | | | | | | | | | |
| Phage- | MDFCPRFL | | | F | | | | F | L | | H | E | | | N | 0.063 | 1.908 | 0.225 | 497 |
| 345 | WHECFN | | | | | | | | | | | | | | | | | | |
| Phage- | MDWCPRHL | | | | | | | H | L | | H | A | | | S | 0.073 | 1.826 | 0.167 | 498 |
| 346 | WHACFS | | | | | | | | | | | | | | | | | | |
| Phage- | MDWCPLEL | | | | | | L | F | L | | D | Q | | | N | 0.116 | 1.819 | 0.264 | 499 |
| 347 | WDQCFN | | | | | | | | | | | | | | | | | | |
| Phage- | MAWCPWYL | | A | | | | W | Y | L | | D | E | | | N | 0.200 | 1.810 | 0.267 | 500 |
| 348 | WDECFN | | | | | | | | | | | | | | | | | | |
| Phage- | LDWCPRHL | L | | | | | | H | L | | A | L | | | N | 0.073 | 1.780 | 0.240 | 501 |
| 349 | WALCFN | | | | | | | | | | | | | | | | | | |
| Phage- | MDWCPWFL | | | | | | W | F | L | | N | E | | | N | 0.158 | 1.774 | 0.279 | 502 |
| 350 | WNECFN | | | | | | | | | | | | | | | | | | |
| Phage- | MDWCPMNL | | | | | | M | N | L | | H | E | | | A | 0.080 | 1.742 | 0.160 | 503 |
| 351 | WHECFA | | | | | | | | | | | | | | | | | | |
| Phage- | MDWCPIHL | | | | | | I | H | L | | Y | E | | | N | 0.080 | 1.734 | 0.203 | 504 |
| 352 | WYECFN | | | | | | | | | | | | | | | | | | |
| Phage- | IDWCPLHL | I | | | | | L | H | L | | H | E | | Y | H | 0.067 | 1.693 | 0.182 | 505 |
| 353 | WHECYH | | | | | | | | | | | | | | | | | | |
| Phage- | MDWCPRYL | | | | | | | Y | L | | L | E | | | N | 0.083 | 1.685 | 0.368 | 506 |
| 354 | WLECFN | | | | | | | | | | | | | | | | | | |
| Phage- | MDWCPMYL | | | | | | M | Y | L | | D | E | | | | 0.082 | 1.652 | 0.178 | 507 |
| 356 | WDECFF | | | | | | | | | | | | | | | | | | |
| Phage- | MDWCPRLL | | | | | | | L | L | | H | E | | | N | 0.071 | 1.601 | 0.109 | 508 |
| 357 | WHECFN | | | | | | | | | | | | | | | | | | |
| Phage- | MDWCPPHL | | | | | | P | H | L | | H | E | | | | 0.069 | 1.449 | 0.136 | 509 |
| 358 | WHECFF | | | | | | | | | | | | | | | | | | |
| Phage- | MDWCPRPL | | | | | | | P | L | | H | E | | | A | 0.063 | 1.439 | 0.138 | 510 |
| 359 | WHECFA | | | | | | | | | | | | | | | | | | |
| Phage- | MDWCPMEL | | | | | | M | F | L | | H | E | | | S | 0.092 | 1.435 | 0.193 | 511 |
| 360 | WHECFS | | | | | | | | | | | | | | | | | | |
| Phage- | MHWCPRHL | | H | | | | | H | L | | S | E | | | N | 0.165 | 1.430 | 0.478 | 512 |
| 361 | WSECFN | | | | | | | | | | | | | | | | | | |
| Phage- | MDWCPRIL | | | | | | | I | L | | H | E | | | S | 0.064 | 1.391 | 0.174 | 513 |
| 362 | WHECFS | | | | | | | | | | | | | | | | | | |
| Phage- | MNWCPMHL | | N | | | | M | H | L | | A | E | | | N | 0.087 | 1.390 | 0.140 | 514 |
| 363 | WAECFN | | | | | | | | | | | | | | | | | | |
| Phage- | MDWCPRSL | | | | | | | S | L | | A | Q | | | Y | 0.067 | 1.387 | 0.140 | 515 |
| 364 | WAQCFY | | | | | | | | | | | | | | | | | | |
| Phage- | MDWCPSYL | | | | | | S | Y | L | | P | V | | | N | 0.079 | 1.178 | 0.131 | 516 |
| 365 | WPVCFN | | | | | | | | | | | | | | | | | | |
| Phage- | MHWCPLYL | | H | | | | L | Y | L | | D | E | | | Y | 0.153 | 1.159 | 0.376 | 517 |
| 366 | WDECFY | | | | | | | | | | | | | | | | | | |
| Phage- | MDWCPSEL | | | | | | S | F | L | | Y | E | | | N | 0.063 | 1.130 | 0.104 | 518 |
| 367 | WYECFN | | | | | | | | | | | | | | | | | | |
| Phage- | MSWCPPYL | | S | | | | P | Y | L | | T | V | | Y | N | 0.176 | 1.104 | 0.326 | 519 |
| 368 | WTVCYN | | | | | | | | | | | | | | | | | | |
| Phage- | MHWCPRDL | | H | | | | | D | L | | Y | E | | | A | 0.072 | 0.995 | 0.108 | S20 |
| 369 | WYECFA | | | | | | | | | | | | | | | | | | |
| Phage- | MTWCPAYL | | T | | | | A | Y | L | | H | E | | | N | 0.086 | 0.985 | 0.139 | 521 |
| 370 | WHECFN | | | | | | | | | | | | | | | | | | |
| Phage- | MAWCPRYL | | A | | | | | Y | L | | A | E | | | | 0.510 | 0.918 | 0.216 | 522 |
| 371 | WAECFF | | | | | | | | | | | | | | | | | | |
| Phage- | MDWCPRYL | | | | | | | Y | L | | | | | | A | 0.095 | 0.917 | 0.218 | 523 |
| 372 | WVDCFA | | | | | | | | | | | | | | | | | | |
| Phage- | MDWCPRIL | | | | | | | I | L | | S | | | | N | 0.082 | 0.847 | 0.113 | 524 |
| 373 | WSDCFN | | | | | | | | | | | | | | | | | | |
| Phage- | MAWCPLDL | | A | | | | L | D | L | | D | K | | | Y | 0.079 | 0.770 | 0.141 | 525 |
| 374 | WDKCFY | | | | | | | | | | | | | | | | | | |
| Phage- | MNWCPRAL | | N | | | | | A | L | | H | E | | | L | 0.085 | 0.759 | 0.107 | 526 |
| 375 | WHECFL | | | | | | | | | | | | | | | | | | |
| Phage- | MDWCPRHL | | | | | | | H | L | | T | Y | | | H | 0.109 | 0.757 | 0.207 | 527 |
| 376 | WTYCFH | | | | | | | | | | | | | | | | | | |
| Phage- | MDWCPFDL | | | | | | F | D | L | | L | E | | | N | 0.087 | 0.650 | 0.120 | 528 |
| 377 | WLECFN | | | | | | | | | | | | | | | | | | |
| Phage- | MHWCPLHL | | H | | | | L | H | L | | N | E | | | A | 0.065 | 0.575 | 0.083 | 529 |
| 378 | WNECFA | | | | | | | | | | | | | | | | | | |
| Phage- | MDWCPRNL | | | | | | | N | L | | A | E | | | S | 0.061 | 0.538 | 0.080 | 530 |
| 379 | WAECFS | | | | | | | | | | | | | | | | | | |
| Phage- | MDYCPSYL | | | Y | | | S | Y | L | | H | E | | | A | 0.065 | 0.470 | 0.090 | 531 |
| 380 | WHECFA | | | | | | | | | | | | | | | | | | |
| Phage- | MAWCPRIL | | A | | | | | I | L | | H | Q | | | N | 0.113 | 0.322 | 0.129 | 532 |
| 381 | WHQCFN | | | | | | | | | | | | | | | | | | |
| Phage- | ITWCPTSL | I | T | | | | T | S | L | | N | R | | L | V | 0.077 | 0.242 | 0.089 | 533 |
| 382 | WNRCLV | | | | | | | | | | | | | | | | | | |
| Phage- | LAGCQRDL | L | A | G | | Q | | D | L | A | T | V | | V | I | 0.068 | 0.229 | 0.116 | 534 |
| 383 | ATVCVI | | | | | | | | | | | | | | | | | | |
| Phage- | IMWCPTSL | I | M | | | | T | S | L | | N | R | | V | T | 0.104 | 0.161 | 0.129 | 535 |
| 384 | WNRCVT | | | | | | | | | | | | | | | | | | |
| Phage- | ILRCQMNL | I | L | R | | Q | M | N | L | Q | D | E | | L | N | 0.083 | 0.083 | 0.077 | 536 |
| 385 | QDECLN | | | | | | | | | | | | | | | | | | |
| Phage- | NHWCPTTL | N | H | | | | T | T | L | | N | R | | V | A | 0.293 | 0.078 | 0.093 | 537 |
| 386 | WNRCVA | | | | | | | | | | | | | | | | | | |
| Phage- | MDWCPRHL | | | | | | | H | L | | L | E | | | N | 0.064 | 0.066 | 0.074 | 538 |
| 387 | WLECFN | | | | | | | | | | | | | | | | | | |
| Phage- | MDWCPRFM | | | | | | | F | M | | N | F | | | | 2.200 | 2.621 | 2.539 | 539 |
| 388 | WNFCFF | | | | | | | | | | | | | | | | | | |
| Phage- | MDWCPSDM | | | | | | S | D | M | | A | N | | | | 0.110 | 2.621 | 0.316 | 540 |
| 389 | WANCFF | | | | | | | | | | | | | | | | | | |
| Phage- | IDWCPMHM | I | | | | | M | H | M | | D | F | | Y | N | 0.078 | 2.544 | 0.232 | S41 |
| 390 | WDFCYN | | | | | | | | | | | | | | | | | | |
| Phage- | MDWCPEDM | | | | | | F | D | M | | A | | | | | 0.063 | 2.532 | 0.215 | 542 |
| 391 | WADCFF | | | | | | | | | | | | | | | | | | |
| Phage- | LHWCPTSM | L | H | | | | T | S | M | | T | Y | | Y | Y | 0.688 | 2.475 | 2.456 | 543 |
| 392 | WTYCYY | | | | | | | | | | | | | | | | | | |
| Phage- | MDWCPRDM | | | | | | | D | M | | F | E | | | N | 0.069 | 2.422 | 0.778 | 544 |
| 393 | WFECFN | | | | | | | | | | | | | | | | | | |
| Phage- | MDWCPRYM | | | | | | | Y | M | | S | | | | | 0.082 | 2.280 | 0.377 | S45 |
| 394 | WSDCFE | | | | | | | | | | | | | | | | | | |
| Phage- | MDWCPKDM | | | | | | K | D | M | | A | E | | | N | 0.083 | 2.106 | 0.457 | 546 |
| 395 | WAECFN | | | | | | | | | | | | | | | | | | |
| Phage- | TDWCPRDM | T | | | | | | D | M | | Y | L | | | N | 0.082 | 2.106 | 0.301 | S47 |
| 396 | WYLCFN | | | | | | | | | | | | | | | | | | |
| Phage- | MDWCPRAM | | | | | | | A | M | | D | Y | | | Y | 0.123 | 2.106 | 0.339 | 548 |
| 397 | WDYCFY | | | | | | | | | | | | | | | | | | |
| Phage- | MDWCPRNM | | | | | | | N | M | | N | E | | | | 0.095 | 2.099 | 0.430 | S49 |
| 398 | WNECFF | | | | | | | | | | | | | | | | | | |
| Phage- | MDWCPRSM | | | | | | | S | M | | D | S | | | N | 0.332 | 2.019 | 0.142 | 550 |
| 399 | WDSCFN | | | | | | | | | | | | | | | | | | |
| Phage- | MHWCPTYM | | H | | | | T | Y | M | | S | E | | | A | 0.070 | 1.839 | 0.161 | 551 |
| 400 | WSECFA | | | | | | | | | | | | | | | | | | |
| Phage- | MDWCPLDM | | | | | | L | D | M | | | L | | | A | 0.085 | 1.837 | 0.169 | 552 |
| 401 | WVLCFA | | | | | | | | | | | | | | | | | | |
| Phage- | MDWCPRHM | | | | | | | H | M | | H | E | | | H | 0.067 | 1.815 | 0.171 | 553 |
| 402 | WHECFH | | | | | | | | | | | | | | | | | | |
| Phage- | MSWCPWDM | | S | | | | W | D | M | | N | E | | | A | 0.069 | 1.521 | 0.108 | 554 |
| 403 | WNECFA | | | | | | | | | | | | | | | | | | |
| Phage- | MDWCPRDM | | | | | | | D | M | | T | S | | | N | 0,100 | 1.414 | 0.179 | 555 |
| 404 | WTSCFN | | | | | | | | | | | | | | | | | | |
| Phage- | MDWCPMSM | | | | | | M | S | M | | A | F | | | D | 0.102 | 1.412 | 0.160 | 556 |
| 405 | WAFCFD | | | | | | | | | | | | | | | | | | |
| Phage- | MNWCPIDM | | N | | | | I | D | M | | T | E | | | N | 0.245 | 1.403 | 0.175 | 557 |
| 406 | WTECFN | | | | | | | | | | | | | | | | | | |
| Phage- | MNWCPIHM | | N | | | | I | H | M | | N | Q | | | A | 0.085 | 1.373 | 0.133 | 558 |
| 407 | WNQCFA | | | | | | | | | | | | | | | | | | |
| Phage- | MEWCPKDM | | F | | | | K | D | M | | A | E | | | A | 0.099 | 1.258 | 0.113 | 559 |
| 408 | WAECFA | | | | | | | | | | | | | | | | | | |
| Phage- | LHWCPITM | L | H | | | | I | T | M | | T | Y | | Y | Y | 0.229 | 1.212 | 0.516 | 560 |
| 409 | WTYCYY | | | | | | | | | | | | | | | | | | |
| Phage- | IDWCPRYM | I | | | | | | Y | M | | H | E | | | | 0.098 | 1.178 | 0.164 | 561 |
| 410 | WHECFF | | | | | | | | | | | | | | | | | | |
| Phage- | MSWCPRFM | | S | | | | | F | M | | H | E | | | N | 0.081 | 1.111 | 0.246 | 562 |
| 411 | WHECFN | | | | | | | | | | | | | | | | | | |
| Phage- | QHWCPRDM | Q | H | | | | | D | M | | N | | | Y | A | 0.066 | 1.076 | 0.149 | 563 |
| 412 | WNDCYA | | | | | | | | | | | | | | | | | | |
| Phage- | MDWCPSDM | | | | | | S | D | M | | S | N | | | N | 0.070 | 1.075 | 0.121 | 564 |
| 413 | WSNCFN | | | | | | | | | | | | | | | | | | |
| Phage- | LYWCPRDM | L | Y | | | | | D | M | | A | Y | | Y | S | 0.068 | 1.019 | 0.157 | 565 |
| 414 | WAYCYS | | | | | | | | | | | | | | | | | | |
| Phage- | MDWCPMYM | | | | | | M | Y | M | | H | K | | | H | 0.099 | 0.987 | 0.168 | 566 |
| 415 | WHKCFH | | | | | | | | | | | | | | | | | | |
| Phage- | MDWCPSNM | | | | | | S | N | M | | N | E | | | | 0.072 | 0.983 | 0.136 | 567 |
| 416 | WNECFF | | | | | | | | | | | | | | | | | | |
| Phage- | IYWCPTAM | I | Y | | | | T | A | M | | N | R | | S | A | 0.086 | 0.817 | 0.073 | 568 |
| 417 | WNRCSA | | | | | | | | | | | | | | | | | | |
| Phage- | MYWCPKYM | | Y | | | | K | Y | M | | S | E | | | A | 0.072 | 0.741 | 0.099 | 569 |
| 418 | WSECFA | | | | | | | | | | | | | | | | | | |
| Phage- | VDWCPAHM | V | | | | | A | H | M | | N | E | | Y | N | 0.208 | 0.664 | 0.085 | 570 |
| 419 | WNECYN | | | | | | | | | | | | | | | | | | |
| Phage- | MEWCPSTM | | F | | | | S | T | M | | N | R | | | D | 0.063 | 0.464 | 0.138 | 571 |
| 420 | WNRCFD | | | | | | | | | | | | | | | | | | |
| Phage- | TIFCPSTM | T | I | F | | | S | T | M | | N | R | | W | T | 0.138 | 0.262 | 0.085 | 572 |
| 421 | WNRCWT | | | | | | | | | | | | | | | | | | |
| Phage- | MYWCPINM | | Y | | | | I | N | M | | D | Y | | Y | A | 0.122 | 0.109 | 0.088 | 573 |
| 422 | WDYCYA | | | | | | | | | | | | | | | | | | |
| Phage- | INLCPTPM | I | N | L | | | T | P | M | | N | R | | W | L | 0.119 | 0.103 | 0.101 | 574 |
| 423 | WNRCWL | | | | | | | | | | | | | | | | | | |
| Phage- | ITQCPSTM | I | T | Q | | | S | T | M | | N | R | | S | V | 0.067 | 0.098 | 0.108 | 575 |
| 424 | WNRCSV | | | | | | | | | | | | | | | | | | |
| Phage- | MDLCPTAM | | | L | | | T | A | M | | N | R | | | Y | 0.063 | 0.097 | 0.084 | 576 |
| 425 | WNRCFY | | | | | | | | | | | | | | | | | | |
| Phage- | MFLCPSAM | | F | L | | | S | A | M | | N | R | | | Y | 0.069 | 0.094 | 0.114 | 577 |
| 426 | WNRCFY | | | | | | | | | | | | | | | | | | |
| Phage- | VILCPTTM | V | I | L | | | T | T | M | | N | R | | | H | 0,100 | 0.072 | 0.084 | 578 |
| 427 | WNRCFH | | | | | | | | | | | | | | | | | | |
| Phage- | MNWCPSTM | | N | | | | S | T | M | | N | R | | L | T | 0.067 | 0.065 | 0.070 | 579 |
| 428 | WNRCLT | | | | | | | | | | | | | | | | | | |
| Phage- | IHLCHWVP | I | H | L | | H | W | V | P | | I | K | | S | H | 2.516 | 2.550 | 2.508 | 580 |
| 430 | WIKCSH | | | | | | | | | | | | | | | | | | |
| Phage- | MAWCPADQ | | A | | | | A | D | Q | | H | E | | | N | 0.076 | 1.845 | 0.456 | 581 |
| 431 | WHECFN | | | | | | | | | | | | | | | | | | |
| Phage- | IDWCPIIQ | I | | | | | I | I | Q | G | L | P | | | A | 0.058 | 0.368 | 0.065 | 582 |
| 432 | GLPCFA | | | | | | | | | | | | | | | | | | |
| Phage- | MAWCPWAQ | | A | | | | W | A | Q | F | D | E | | L | A | 0.073 | 0.071 | 0.078 | 583 |
| 433 | FDECLA | | | | | | | | | | | | | | | | | | |
| Phage- | IVWCPTTQ | I | V | | | | T | T | Q | | N | R | | A | T | 0.113 | 0.067 | 0.083 | 584 |
| 434 | WNRCAT | | | | | | | | | | | | | | | | | | |
| Phage- | MDWCPRDR | | | | | | | D | | | | | | Y | A | 0.157 | 1.484 | 0.403 | 585 |
| 435 | WVDCYA | | | | | | | | | | | | | | | | | | |
| Phage- | MDWCPRDR | | | | | | | D | | | A | | | | N | 0.063 | 1.351 | 0.080 | 586 |
| 436 | WADCFN | | | | | | | | | | | | | | | | | | |
| Phage- | MDWCPVDR | | | | | | V | D | | | A | | | | N | 0.071 | 1.156 | 0.315 | 587 |
| 437 | WADCFN | | | | | | | | | | | | | | | | | | |
| Phage- | MDWCPMSR | | | | | | M | S | | | A | F | | | D | 0.071 | 1.000 | 0.115 | 588 |
| 438 | WAFCFD | | | | | | | | | | | | | | | | | | |
| Phage- | MDWCPRDR | | | | | | | D | | | D | V | | | A | 0.073 | 0.924 | 0.096 | 589 |
| 439 | WDVCFA | | | | | | | | | | | | | | | | | | |
| Phage- | MDWCPRDR | | | | | | | D | | | | | | | N | 0.106 | 0.911 | 0.132 | 590 |
| 440 | WVDCFN | | | | | | | | | | | | | | | | | | |
| Phage- | MDWCPRDR | | | | | | | D | | | D | | | | | 0.114 | 0.899 | 0.183 | 591 |
| 441 | WDDCFF | | | | | | | | | | | | | | | | | | |
| Phage- | IYWCPIDR | I | Y | | | | I | D | | | N | | | Y | N | 0.177 | 0.867 | 0.164 | 592 |
| 442 | WNDCYN | | | | | | | | | | | | | | | | | | |
| Phage- | MDWCPMTR | | | | | | M | T | | | N | | | Y | | 0.081 | 0.671 | 0.078 | 593 |
| 443 | WNDCYF | | | | | | | | | | | | | | | | | | |
| Phage- | VNQCTRYR | V | N | Q | | T | | Y | | | A | E | | L | N | 0.465 | 0.566 | 0.135 | 594 |
| 444 | WAECLN | | | | | | | | | | | | | | | | | | |
| Phage- | MDWCPRAR | | | | | | | A | | | H | | | | | 0.097 | 0.497 | 0.138 | 595 |
| 445 | WHDCFE | | | | | | | | | | | | | | | | | | |
| Phage- | MDWCPRDR | | | | | | | D | | | D | | | | N | 0.069 | 0.450 | 0.077 | 596 |
| 446 | WDDCFN | | | | | | | | | | | | | | | | | | |
| Phage- | MDWCPRDR | | | | | | | D | | | D | V | | Y | Y | 0.073 | 0.074 | 0.073 | 597 |
| 447 | WDVCYY | | | | | | | | | | | | | | | | | | |
| Phage- | MAWCPWAS | | A | | | | W | A | S | F | D | E | | L | A | 0.104 | 0.089 | 0.109 | 598 |
| 448 | FDECLA | | | | | | | | | | | | | | | | | | |
| Phage- | MVDCPLIS | | V | D | | | L | I | S | F | D | E | | L | A | 0.246 | 0.070 | 0.076 | 599 |
| 449 | FDECLA | | | | | | | | | | | | | | | | | | |
| Phage- | EDWCPTDV | E | | | | | T | D | V | | P | Y | | | S | 0.100 | 0.711 | 0.157 | 600 |
| 450 | WPYCFS | | | | | | | | | | | | | | | | | | |
| Phage- | MDWCPREW | | | | | | | F | W | | H | E | | Y | A | 0.246 | 2.397 | 0.503 | 601 |
| 451 | WHECYA | | | | | | | | | | | | | | | | | | |
| Phage- | MDWCPRDW | | | | | | | D | W | | H | M | | | N | 0.100 | 2.115 | 0.243 | 602 |
| 452 | WHMCFN | | | | | | | | | | | | | | | | | | |
| Phage- | MDWCPSDY | | | | | | S | D | Y | | Y | V | | | A | 0.065 | 0.707 | 0,080 | 603 |
| 453 | WYVCFA | | | | | | | | | | | | | | | | | | |

TABLE 7
Phage Panning Results of Peptide-87 Library Sequences. (—) indicates
the same amino acid position as in Peptide-87 corresponding position.

| | | | | | | | | | | | | | | | Phase binding ELISA | | | |
|---|---|---|---|---|---|---|---|---|---|---|---|---|---|---|---|---|---|---|
| | | | | | | | | | | | | | | | | | Anti- | |
| | | | | | | | | | | | | | | | | | CD28 | |
| | | | | | | | | | | | | | | | | Anti- | Fab | |
| | | | | | | | | | | | | | | | | CD28 | signal | |
| | | | | | | | | | | | | | | | | Fab | in | |
| | | | | | | | | | | | | | | | | (SEQ ID | the | |
| | | | | | | | | | | | | | | | | NOs: | presence | SEQ |
| | Amino acid position sequence | | | | | | | | | | | | | | Background | 696, 697) | of | ID |
| Phage ID | 1 | 2 | 3 | 4 | 5 | 6 | 7 | 8 | 9 | 10 | 11 | 12 | 13 | 14 | signal | signal | CD28 | NO: |
| Phage-466 | Y | C | A | G | H | A | W | Q | Y | I | N | K | C | L | 0.06 | 0.64 | 0.07 | 744 |
| Phage-454 | — | W | S | C | F | Q | I | P | F | T | C | F | Y | F | 0.18 | 1.44 | 0.27 | 732 |
| Phage-455 | W | E | C | L | K | Y | T | E | A | N | A | C | T | Y | 0.05 | 1.55 | 0.07 | 733 |
| Phage-456 | F | M | C | L | P | Q | N | P | H | P | L | C | F | K | 0.06 | 0.84 | 0.06 | 734 |
| Phage-457 | P | D | C | Q | T | I | L | F | K | P | W | C | V | R | 0.05 | 2.09 | 0.06 | 735 |
| Phage-458 | A | — | D | T | Y | F | A | G | M | — | — | E | — | F | 0.07 | 0.92 | 0.10 | 736 |
| Phage-459 | G | — | Q | Y | P | W | A | F | D | A | M | S | — | P | 0.05 | 2.38 | 0.07 | 737 |
| Phage-460 | W | — | W | D | Y | K | L | W | V | P | T | Q | — | P | 0.11 | 2.27 | 0.14 | 738 |
| Phage-461 | W | — | W | D | Y | R | E | W | V | P | M | T | — | P | 0.06 | 2.30 | 0.08 | 739 |
| Phage-462 | E | — | H | W | S | M | — | A | V | G | H | A | — | F | 0.06 | 1.77 | 0.07 | 740 |
| Phage-463 | — | — | — | — | — | S | P | I | N | — | G | — | — | — | 0.05 | 1.49 | 0.06 | 741 |
| Phage-464 | W | — | W | D | Y | K | L | W | V | P | M | T | — | P | 0.11 | 2.51 | 0.11 | 742 |
| Phage-465 | D | — | W | W | M | F | E | S | — | P | G | R | — | I | 0.06 | 1.65 | 0.07 | 743 |
| Phage-467 | W | — | W | Y | Y | R | E | W | V | P | M | T | — | P | 0.06 | 1.85 | 0.07 | 745 |
| Phage-468 | — | W | Y | C | S | P | S | I | V | R | C | V | L | V | 0.06 | 1.51 | 0.09 | 746 |
| Phage-469 | P | D | C | H | T | I | L | F | K | P | W | Q | V | R | 0.07 | 1.00 | 0.06 | 747 |
| Phage-470 | W | I | C | D | K | S | G | S | I | M | L | C | R | A | 0.06 | 0.91 | 0.06 | 748 |
| Phage-471 | S | — | — | A | G | — | S | Y | A | W | P | Y | — | — | 0.05 | 1.49 | 0.05 | 749 |
| Phage-472 | D | — | P | W | L | S | G | F | S | R | P | L | L | C | 0.09 | 1.27 | 0.07 | 750 |
| Phage-473 | — | — | — | — | M | S | T | I | Q | L | W | L | K | C | 0.06 | 2.16 | 0.09 | 751 |
| Phage-474 | — | — | — | — | — | M | P | — | — | E | V | — | — | — | 0.07 | 0.22 | 0.06 | 752 |
| Phage-475 | — | — | — | — | — | T | E | E | H | Y | V | — | — | M | 0.05 | 0.62 | 0.07 | 753 |
| Phage-476 | — | — | M | — | — | S | H | E | H | — | W | — | — | — | 0.06 | 0.87 | 0.07 | 754 |
| Phage-477 | — | — | — | — | — | S | H | E | — | L | A | — | — | E | 0.05 | 0.86 | 0.06 | 755 |
| Phage-478 | — | — | E | — | — | S | P | E | — | — | L | — | — | — | 0.05 | 1.75 | 0.06 | 774 |
| Phage-479 | — | — | E | — | — | R | P | E | — | — | L | — | — | — | 0.06 | 1.27 | 0.06 | 775 |
| Phage-480 | — | — | E | — | — | E | G | I | — | F | W | — | — | — | 0.05 | 2.07 | 0.06 | 776 |
| Phage-481 | — | — | E | — | — | L | P | E | — | — | E | — | — | — | 0.05 | 2.06 | 0.08 | 777 |
| Phage-482 | — | — | E | — | — | — | P | E | — | — | A | — | — | — | 0.05 | 2.15 | 0.09 | 778 |
| Phage-483 | — | — | E | — | — | — | P | E | — | — | R | — | — | — | 0.06 | 1.63 | 0.08 | 779 |
| Phage-484 | — | — | E | — | — | — | P | E | — | — | W | — | — | — | 0.08 | 1.63 | 0.07 | 780 |
| Phage-485 | — | — | E | — | — | S | Q | E | N | — | W | — | — | — | 0.05 | 1.41 | 0.07 | 781 |
| Phage-486 | — | — | E | — | — | L | P | E | — | L | G | — | — | — | 0.06 | 1.23 | 0.08 | 782 |
| Phage-487 | — | — | E | — | — | L | P | E | — | R | E | — | — | — | 0.06 | 1.96 | 0.07 | 783 |
| Phage-488 | — | — | E | — | — | L | P | E | — | L | A | — | — | — | 0.06 | 2.34 | 0.10 | 784 |
| Phage-489 | — | — | E | — | — | S | Q | E | — | L | W | — | — | — | 0.07 | 2.36 | 0.07 | 785 |
| Phage-490 | — | — | E | — | — | S | S | E | — | — | Q | — | — | — | 0.06 | 2.25 | 0.10 | 786 |
| Phage-491 | H | — | S | — | — | H | — | L | Q | V | W | — | — | F | 0.06 | 2.31 | 0.07 | 787 |
| Phage-492 | — | — | W | S | — | S | P | P | F | — | K | — | — | Y | 0.07 | 2.06 | 0.06 | 788 |
| Phage-493 | — | — | E | — | — | L | P | E | — | R | — | — | — | — | 0.08 | 2.38 | 0.17 | 789 |
| Phage-494 | — | — | E | — | — | L | P | E | — | L | E | — | — | V | 0.07 | 2.11 | 0.06 | 790 |
| Phage-495 | — | — | E | — | — | L | P | E | — | — | Q | — | — | — | 0.06 | 1.90 | 0.08 | 791 |
| Phage-496 | — | — | E | — | — | L | P | E | — | — | W | — | — | — | 0.09 | 2.32 | 0.09 | 792 |
| Phage-497 | — | — | E | — | — | — | P | E | — | — | G | — | — | — | 0.10 | 2.10 | 0.07 | 793 |
| Phage-498 | — | — | D | — | — | S | P | I | — | M | W | — | — | — | 0.08 | 2.47 | 0.09 | 814 |
| Phage-499 | — | — | E | — | — | — | P | E | — | M | A | — | — | — | 0.07 | 2.47 | 0.08 | 815 |
| Phage-500 | — | — | D | — | — | L | P | — | — | M | E | — | — | — | 0.06 | 2.47 | 0.11 | 816 |
| Phage-501 | — | — | — | — | — | — | P | E | — | M | E | — | — | — | 0.06 | 2.45 | 0.09 | 817 |
| Phage-502 | — | — | D | — | — | S | P | E | — | M | W | — | — | — | 0.07 | 2.44 | 0.09 | 818 |
| Phage-503 | — | — | D | — | — | L | P | E | — | M | E | — | — | — | 0.08 | 2.44 | 0.10 | 819 |
| Phage-504 | — | — | E | — | — | L | P | — | — | M | W | — | — | — | 0.11 | 2.44 | 0.17 | 820 |
| Phage-505 | — | — | D | — | — | L | P | E | — | M | — | — | — | — | 0.06 | 2.43 | 0.13 | 821 |
| Phage-506 | — | — | — | — | — | L | P | E | — | M | D | — | — | — | 0.07 | 2.42 | 0.12 | 822 |
| Phage-507 | — | — | D | — | — | S | A | E | H | M | A | — | — | — | 0.07 | 2.41 | 0.08 | 823 |
| Phage-508 | — | — | — | — | — | L | P | — | — | M | A | — | — | — | 0.07 | 2.41 | 0.17 | 824 |
| Phage-509 | — | — | E | — | — | L | P | E | — | M | A | — | — | — | 0.07 | 2.41 | 0.14 | 825 |
| Phage-510 | — | — | E | — | — | L | P | E | — | M | F | — | — | — | 0.10 | 2.40 | 0.12 | 826 |
| Phage-511 | — | — | D | — | — | L | P | — | — | M | A | Q | — | — | 0.06 | 2.40 | 0.20 | 827 |
| Phage-512 | — | — | E | — | — | L | P | — | — | M | Q | — | — | — | 0.06 | 2.39 | 0.12 | 828 |
| Phage-513 | — | — | D | — | — | — | P | E | — | M | M | — | — | — | 0.10 | 2.39 | 0.09 | 829 |
| Phage-514 | — | — | E | — | — | L | P | E | — | M | W | — | — | — | 0.07 | 2.39 | 0.08 | 830 |
| Phage-515 | — | — | E | — | — | — | A | E | — | M | A | — | — | — | 0.07 | 2.38 | 0.08 | 831 |
| Phage-516 | — | — | D | — | — | L | P | E | — | L | A | — | — | — | 0.08 | 2.38 | 0.11 | 832 |
| Phage-517 | — | — | — | — | — | M | P | E | — | M | A | — | — | — | 0.10 | 2.38 | 0.11 | 833 |
| Phage-518 | — | — | D | — | — | L | P | E | — | M | S | — | — | M | 0.10 | 2.38 | 0.14 | 834 |
| Phage-519 | — | — | E | — | — | T | A | E | — | M | Q | — | — | — | 0.08 | 2.38 | 0.08 | 835 |
| Phage-520 | — | — | M | — | — | — | P | E | — | M | A | — | — | — | 0.11 | 2.38 | 0.09 | 836 |
| Phage-521 | — | — | D | — | — | L | P | E | — | M | A | — | — | — | 0.07 | 2.37 | 0.13 | 837 |
| Phage-522 | — | — | D | — | — | S | — | E | — | — | G | — | — | — | 0.08 | 2.37 | 0.07 | 838 |
| Phage-523 | — | — | D | — | — | L | P | E | — | Q | E | — | — | — | 0.06 | 2.37 | 0.10 | 839 |
| Phage-524 | — | — | — | — | — | L | P | — | — | M | S | — | — | — | 0.06 | 2.37 | 0.11 | 840 |
| Phage-525 | — | — | D | — | — | L | P | I | — | M | S | Q | — | — | 0.07 | 2.36 | 0.12 | 841 |
| Phage-526 | — | — | E | — | — | S | P | E | — | M | A | — | — | — | 0.06 | 2.35 | 0.07 | 842 |
| Phage-527 | — | — | D | — | — | L | P | E | — | M | S | — | — | — | 0.07 | 2.35 | 0.14 | 843 |
| Phage-528 | — | — | D | — | — | L | P | — | — | M | A | — | — | — | 0.07 | 2.35 | 0.15 | 844 |
| Phage-529 | — | — | Q | — | — | L | P | M | — | M | A | — | — | — | 0.06 | 2.34 | 0.08 | 845 |
| Phage-530 | — | — | D | — | — | L | P | E | — | M | M | — | — | — | 0.07 | 2.34 | 0.11 | 846 |
| Phage-531 | — | — | D | — | — | S | P | M | — | — | L | — | — | — | 0.07 | 2.34 | 0.08 | 847 |
| Phage-532 | — | — | Q | — | — | L | P | M | — | M | E | — | — | — | 0.09 | 2.34 | 0.09 | 848 |
| Phage-533 | — | — | E | — | — | L | P | — | — | M | A | — | — | — | 0.07 | 2.34 | 0.14 | 849 |
| Phage-534 | — | — | D | — | — | S | P | L | — | M | W | — | — | — | 0.06 | 2.34 | 0.08 | 850 |
| Phage-535 | — | — | D | — | — | L | P | M | — | M | Q | — | — | — | 0.06 | 2.33 | 0.11 | 851 |
| Phage-536 | — | — | D | — | — | L | P | I | — | M | S | — | — | — | 0.07 | 2.32 | 0.08 | 852 |
| Phage-537 | — | — | E | — | — | S | — | A | N | L | G | — | — | — | 0.07 | 2.32 | 0.08 | 853 |
| Phage-538 | — | — | D | — | — | L | P | E | — | R | E | — | — | F | 0.07 | 2.32 | 0.07 | 854 |
| Phage-539 | — | — | E | — | — | L | P | E | — | M | L | — | — | — | 0.06 | 2.32 | 0.09 | 855 |
| Phage-540 | — | — | D | — | — | E | P | — | — | M | L | — | — | — | 0.09 | 2.32 | 0.12 | 856 |
| Phage-541 | — | — | E | — | — | S | P | E | — | M | S | — | — | — | 0.07 | 2.31 | 0.07 | 857 |
| Phage-542 | — | — | E | — | — | T | M | E | — | M | W | — | — | — | 0.06 | 2.31 | 0.07 | 858 |
| Phage-543 | — | — | — | — | — | L | P | E | — | M | P | H | — | I | 0.07 | 2.31 | 0.09 | 859 |
| Phage-544 | — | — | D | — | — | S | P | — | — | — | E | — | — | — | 0.07 | 2.31 | 0.07 | 860 |
| Phage-545 | — | — | D | — | — | S | P | E | — | — | L | — | — | — | 0.06 | 2.31 | 0.09 | 861 |
| Phage-546 | — | — | D | — | — | L | D | E | — | M | A | — | — | — | 0.07 | 2.31 | 0.08 | 862 |
| Phage-547 | — | — | D | — | — | T | L | E | — | M | W | — | — | — | 0.07 | 2.31 | 0.13 | 863 |
| Phage-548 | — | — | E | — | — | L | P | L | — | M | W | — | — | — | 0.07 | 2.30 | 0.07 | 864 |
| Phage-549 | — | — | D | — | — | — | P | E | — | M | A | — | — | — | 0.07 | 2.30 | 0.09 | 865 |
| Phage-550 | — | — | — | — | — | S | P | E | H | M | W | — | — | — | 0.06 | 2.30 | 0.06 | 866 |
| Phage-551 | — | — | E | — | — | S | E | E | — | — | S | — | — | — | 0.07 | 2.30 | 0.08 | 867 |
| Phage-552 | — | — | D | — | — | L | P | I | — | M | A | — | — | — | 0.07 | 2.30 | 0.14 | 868 |
| Phage-553 | — | — | D | — | — | — | P | — | — | M | R | — | — | — | 0.05 | 2.30 | 0.07 | 869 |
| Phage-554 | — | — | D | — | — | — | K | E | — | M | W | — | — | — | 0.07 | 2.30 | 0.07 | 870 |
| Phage-555 | — | — | E | — | — | S | S | E | — | M | L | — | — | — | 0.06 | 2.30 | 0.07 | 871 |
| Phage-556 | — | — | E | — | — | — | A | E | — | M | E | — | — | — | 0.07 | 2.30 | 0.11 | 872 |
| Phage-557 | — | — | E | — | — | S | A | — | — | M | S | — | — | — | 0.07 | 2.29 | 0.07 | 873 |
| Phage-558 | — | — | D | — | — | S | R | E | — | — | A | — | — | — | 0.06 | 2.29 | 0.08 | 874 |
| Phage-559 | — | — | D | — | — | L | P | — | — | M | W | — | — | — | 0.07 | 2.29 | 0.12 | 875 |
| Phage-560 | — | — | D | — | — | V | P | E | — | M | A | — | — | — | 0.08 | 2.29 | 0.07 | 876 |
| Phage-561 | — | — | D | — | — | L | P | M | — | M | M | — | — | — | 0.07 | 2.28 | 0.14 | 877 |
| Phage-562 | — | — | Q | — | — | — | P | E | — | M | A | — | — | — | 0.08 | 2.28 | 0.10 | 878 |
| Phage-563 | — | — | D | — | — | S | Q | E | — | M | M | — | — | — | 0.06 | 2.28 | 0.06 | 879 |
| Phage-564 | — | — | S | — | — | L | P | — | — | M | Q | — | — | — | 0.06 | 2.28 | 0.08 | 880 |
| Phage-565 | — | — | D | — | — | S | S | E | — | M | A | — | — | — | 0.07 | 2.28 | 0.09 | 881 |
| Phage-566 | — | — | D | — | — | — | P | — | — | M | Q | — | — | — | 0.07 | 2.28 | 0.11 | 882 |
| Phage-567 | — | — | E | — | — | L | P | I | — | L | E | — | — | M | 0.06 | 2.27 | 0.10 | 883 |
| Phage-568 | — | — | E | — | — | T | P | E | — | M | Q | — | — | — | 0.07 | 2.27 | 0.07 | 884 |
| Phage-569 | — | — | D | — | — | L | P | — | — | M | M | — | — | — | 0.07 | 2.27 | 0.14 | 885 |
| Phage-570 | — | — | D | — | — | S | T | E | — | L | W | — | — | — | 0.06 | 2.26 | 0.06 | 886 |
| Phage-571 | — | — | E | — | — | L | G | I | — | M | Y | — | — | — | 0.07 | 2.26 | 0.14 | 887 |
| Phage-572 | — | — | D | — | — | — | A | E | — | M | A | — | — | — | 0.07 | 2.26 | 0.13 | 888 |
| Phage-573 | — | — | E | — | — | S | Q | E | — | M | S | — | — | — | 0.07 | 2.26 | 0.07 | 889 |
| Phage-574 | — | — | D | — | — | L | P | E | — | L | W | — | — | — | 0.07 | 2.26 | 0.13 | 890 |
| Phage-575 | — | — | E | — | — | L | P | M | — | M | E | — | — | — | 0.07 | 2.25 | 0.10 | 891 |
| Phage-576 | — | — | D | — | — | L | S | E | — | M | E | — | — | — | 0.07 | 2.25 | 0.08 | 892 |
| Phage-577 | — | — | E | — | — | S | P | L | — | M | W | — | — | — | 0.06 | 2.25 | 0.06 | 893 |
| Phage-578 | — | — | E | — | — | S | P | E | — | M | E | — | — | — | 0.06 | 2.24 | 0.07 | 894 |
| Phage-579 | — | — | — | — | — | L | P | E | — | M | E | — | — | — | 0.08 | 2.24 | 0.12 | 895 |
| Phage-580 | — | — | E | — | — | — | — | E | — | M | A | — | — | — | 0.09 | 2.24 | 0.07 | 896 |
| Phage-581 | — | — | Q | — | — | L | P | E | — | M | — | — | — | — | 0.06 | 2.24 | 0.08 | 897 |
| Phage-582 | — | — | D | — | — | S | Q | E | — | M | A | — | — | — | 0.05 | 2.24 | 0.08 | 898 |
| Phage-583 | — | — | Q | — | — | L | P | I | — | M | E | — | — | — | 0.06 | 2.24 | 0.09 | 899 |
| Phage-584 | — | — | D | — | — | L | P | L | — | L | A | — | — | — | 0.05 | 2.23 | 0.09 | 900 |
| Phage-585 | — | — | D | — | — | L | P | — | — | M | — | — | — | — | 0.05 | 2.23 | 0.10 | 901 |
| Phage-586 | — | — | E | — | — | — | P | M | — | M | W | — | — | — | 0.06 | 2.23 | 0.08 | 902 |
| Phage-587 | — | — | E | — | — | L | P | E | — | M | Q | — | — | M | 0.06 | 2.23 | 0.08 | 903 |
| Phage-588 | — | — | D | — | — | S | A | E | — | M | A | — | — | — | 0.08 | 2.23 | 0.07 | 904 |
| Phage-589 | — | — | E | — | — | S | P | E | — | M | Y | — | — | — | 0.06 | 2.23 | 0.10 | 905 |
| Phage-590 | — | — | D | — | — | S | M | E | — | M | S | — | — | — | 0.08 | 2.22 | 0.09 | 906 |
| Phage-591 | — | — | E | — | — | L | P | M | — | M | D | — | — | — | 0.06 | 2.22 | 0.07 | 907 |
| Phage-592 | — | — | D | — | — | R | G | E | — | L | Q | — | — | — | 0.07 | 2.22 | 0.09 | 908 |
| Phage-593 | — | — | E | — | — | S | P | — | — | M | W | — | — | — | 0.08 | 2.22 | 0.12 | 909 |
| Phage-594 | — | — | E | — | — | — | A | E | — | M | T | — | — | — | 0.07 | 2.22 | 0.08 | 910 |
| Phage-595 | — | — | E | — | — | L | P | — | — | M | K | — | — | — | 0.08 | 2.22 | 0.10 | 911 |
| Phage-596 | — | — | D | — | — | I | E | E | — | R | A | — | — | — | 0.06 | 2.21 | 0.07 | 912 |
| Phage-597 | — | — | E | — | — | — | P | E | — | M | L | — | — | — | 0.06 | 2.21 | 0.07 | 913 |
| Phage-598 | — | — | D | — | — | — | P | E | — | M | E | — | — | M | 0.08 | 2.21 | 0.07 | 914 |
| Phage-599 | — | — | E | — | — | T | V | E | — | M | A | — | — | — | 0.08 | 2.20 | 0.08 | 915 |
| Phage-600 | — | — | D | — | — | K | A | E | — | M | D | — | — | — | 0.07 | 2.20 | 0.07 | 916 |
| Phage-601 | — | — | D | — | — | — | P | E | — | M | L | — | — | — | 0.05 | 2.20 | 0.07 | 917 |
| Phage-602 | — | — | D | — | — | — | P | D | — | M | L | — | — | — | 0.05 | 2.20 | 0.09 | 918 |
| Phage-603 | — | — | D | — | — | L | P | E | — | L | P | — | — | — | 0.07 | 2.19 | 0.09 | 919 |
| Phage-604 | — | — | D | — | — | M | P | E | — | — | A | — | — | — | 0.07 | 2.19 | 0.08 | 920 |
| Phage-605 | — | — | D | — | — | L | P | E | — | L | S | — | — | — | 0.07 | 2.19 | 0.07 | 921 |
| Phage-606 | — | — | — | — | — | L | P | E | — | F | L | — | — | — | 0.07 | 2.19 | 0.08 | 922 |
| Phage-607 | — | — | D | — | — | S | P | L | — | — | W | — | — | — | 0.07 | 2.19 | 0.09 | 923 |
| Phage-608 | — | — | D | — | — | L | P | E | — | M | H | — | — | — | 0.05 | 2.19 | 0.09 | 924 |
| Phage-609 | — | — | D | — | — | L | P | E | — | — | E | — | — | — | 0.06 | 2.19 | 0.07 | 925 |
| Phage-610 | — | — | D | — | — | L | P | — | — | K | A | — | — | — | 0.07 | 2.19 | 0.09 | 926 |
| Phage-611 | — | — | D | — | — | — | V | E | — | M | A | — | — | — | 0.07 | 2.18 | 0.10 | 927 |
| Phage-612 | — | — | D | — | — | S | I | E | — | R | W | — | — | — | 0.06 | 2.18 | 0.06 | 928 |
| Phage-613 | — | — | E | — | — | — | E | E | — | M | Q | — | — | — | 0.06 | 2.18 | 0.06 | 929 |
| Phage-614 | — | — | D | — | — | — | P | E | — | — | Y | — | — | — | 0.06 | 2.18 | 0.06 | 930 |
| Phage-615 | — | — | D | — | — | L | E | E | — | K | Q | — | — | — | 0.07 | 2.18 | 0.08 | 931 |
| Phage-616 | — | — | M | — | — | L | P | — | — | M | Q | — | — | — | 0.09 | 2.18 | 0.11 | 932 |
| Phage-617 | — | — | D | — | — | T | Q | E | — | — | A | — | — | — | 0.05 | 2.17 | 0.06 | 933 |
| Phage-618 | — | — | D | — | — | T | Q | E | — | M | A | — | — | — | 0.08 | 2.17 | 0.07 | 934 |
| Phage-619 | — | — | E | — | — | L | P | V | — | M | W | — | — | — | 0.07 | 2.17 | 0.09 | 935 |
| Phage-620 | — | — | D | — | — | M | P | E | — | M | W | — | — | — | 0.07 | 2.17 | 0.09 | 936 |
| Phage-621 | — | — | — | — | — | S | P | E | — | M | T | — | — | — | 0.05 | 2.17 | 0.09 | 937 |
| Phage-622 | — | — | — | — | — | — | P | E | — | — | Q | — | — | — | 0.06 | 2.17 | 0.09 | 938 |
| Phage-623 | — | — | D | — | — | V | A | E | — | R | D | — | — | — | 0.09 | 2.17 | 0.07 | 939 |
| Phage-624 | — | — | — | — | — | — | P | E | — | M | Q | — | — | — | 0.06 | 2.16 | 0.07 | 940 |
| Phage-625 | — | — | D | — | — | S | N | E | — | M | A | — | — | — | 0.06 | 2.16 | 0.07 | 941 |
| Phage-626 | — | — | E | — | — | L | P | — | — | L | Q | — | — | — | 0.07 | 2.16 | 0.09 | 942 |
| Phage-627 | — | — | D | — | — | V | A | E | — | M | A | — | — | — | 0.15 | 2.16 | 0.07 | 943 |
| Phage-628 | — | — | D | — | — | L | P | E | — | M | W | — | — | I | 0.07 | 2.15 | 0.07 | 944 |
| Phage-629 | — | — | D | — | — | S | A | E | — | M | W | — | — | — | 0.06 | 2.15 | 0.08 | 945 |
| Phage-630 | — | — | D | — | — | S | L | E | — | M | A | — | — | — | 0.05 | 2.15 | 0.06 | 946 |
| Phage-631 | — | — | E | — | — | L | P | E | — | M | S | — | — | — | 0.06 | 2.15 | 0.07 | 947 |
| Phage-632 | — | — | — | — | — | L | P | E | — | M | Q | — | — | — | 0.17 | 2.15 | 0.09 | 948 |
| Phage-633 | — | — | D | — | — | L | G | I | — | M | W | — | — | — | 0.06 | 2.14 | 0.08 | 949 |
| Phage-634 | — | — | E | — | — | S | A | E | — | M | A | — | — | — | 0.05 | 2.14 | 0.06 | 950 |
| Phage-635 | — | — | D | — | — | S | D | E | — | M | A | — | — | — | 0.06 | 2.14 | 0.07 | 951 |
| Phage-636 | — | — | E | — | — | S | L | E | — | — | G | — | — | — | 0.06 | 2.14 | 0.06 | 952 |
| Phage-637 | — | — | D | — | — | Q | P | E | — | M | A | — | — | — | 0.07 | 2.14 | 0.07 | 953 |
| Phage-638 | — | — | — | — | — | L | P | E | — | — | S | — | — | — | 0.06 | 2.14 | 0.08 | 954 |
| Phage-639 | — | — | D | — | — | I | P | I | — | M | S | — | — | — | 0.07 | 2.13 | 0.08 | 955 |
| Phage-640 | — | — | E | — | — | L | P | — | — | L | A | — | — | — | 0.07 | 2.12 | 0.10 | 956 |
| Phage-641 | — | — | — | — | — | L | P | L | — | M | E | — | — | — | 0.06 | 2.12 | 0.06 | 957 |
| Phage-642 | — | — | D | — | — | S | A | E | — | M | M | — | — | — | 0.08 | 2.12 | 0.08 | 958 |
| Phage-643 | — | — | E | — | — | L | P | — | — | — | P | — | — | — | 0.07 | 2.12 | 0.07 | 959 |
| Phage-644 | — | — | D | — | — | — | P | — | — | M | L | — | — | — | 0.07 | 2.12 | 0.09 | 960 |
| Phage-645 | — | — | D | — | — | L | P | V | — | M | A | — | — | — | 0.07 | 2.11 | 0.09 | 961 |
| Phage-646 | — | — | M | — | — | S | E | E | — | M | Q | — | — | — | 0.07 | 2.11 | 0.07 | 962 |
| Phage-647 | — | — | D | — | — | L | P | E | — | M | H | — | — | — | 0.08 | 2.11 | 0.11 | 963 |
| Phage-648 | — | — | D | — | — | L | E | E | — | M | A | — | — | — | 0.07 | 2.11 | 0.09 | 964 |
| Phage-649 | — | — | D | — | — | M | P | E | — | M | L | — | — | — | 0.07 | 2.11 | 0.09 | 965 |
| Phage-650 | — | — | S | — | — | — | P | I | — | M | A | — | — | — | 0.08 | 2.10 | 0.07 | 966 |
| Phage-651 | — | — | — | — | — | T | Q | E | — | M | S | — | — | — | 0.06 | 2.10 | 0.05 | 967 |
| Phage-652 | — | — | E | — | — | S | P | — | — | M | Q | — | — | — | 0.06 | 2.10 | 0.06 | 968 |
| Phage-653 | — | — | D | — | — | V | P | — | — | M | A | — | — | — | 0.08 | 2.10 | 0.07 | 969 |
| Phage-654 | — | — | D | — | — | S | A | E | — | M | S | — | — | — | 0.06 | 2.10 | 0.06 | 970 |
| Phage-655 | — | — | Q | — | — | L | P | — | — | — | A | — | — | — | 0.06 | 2.09 | 0.08 | 971 |
| Phage-656 | — | — | — | — | — | L | P | — | — | M | E | — | — | M | 0.06 | 2.09 | 0.08 | 972 |
| Phage-657 | — | — | — | — | — | L | P | E | H | — | G | — | — | — | 0.05 | 2.09 | 0.07 | 973 |
| Phage-658 | — | — | E | — | — | T | A | E | — | L | Q | — | — | — | 0.07 | 2.09 | 0.10 | 974 |
| Phage-659 | — | — | — | — | — | S | P | E | — | M | G | — | — | M | 0.07 | 2.09 | 0.06 | 975 |
| Phage-660 | — | — | S | — | — | L | P | E | — | M | E | — | — | — | 0.08 | 2.09 | 0.09 | 976 |
| Phage-661 | — | — | — | — | — | L | E | I | — | M | Q | — | — | — | 0.07 | 2.09 | 0.08 | 977 |
| Phage-662 | — | — | M | — | — | L | P | — | — | M | E | — | — | — | 0.06 | 2.08 | 0.08 | 978 |
| Phage-663 | — | — | E | — | — | S | A | E | — | M | W | — | — | — | 0.06 | 2.08 | 0.09 | 979 |
| Phage-664 | — | — | E | — | — | — | P | E | H | M | A | — | — | — | 0.08 | 2.08 | 0.10 | 980 |
| Phage-665 | — | — | D | — | — | — | E | — | — | M | E | — | — | — | 0.06 | 2.08 | 0.07 | 981 |
| Phage-666 | — | — | E | — | — | S | P | E | — | M | S | H | — | F | 0.06 | 2.08 | 0.09 | 982 |
| Phage-667 | — | — | E | — | — | S | M | — | H | M | A | — | — | — | 0.06 | 2.07 | 0.08 | 983 |
| Phage-668 | — | — | D | — | — | — | Q | E | F | M | S | — | — | — | 0.07 | 2.07 | 0.12 | 984 |
| Phage-669 | — | — | — | — | — | L | P | E | — | — | D | — | — | — | 0.06 | 2.07 | 0.08 | 985 |
| Phage-670 | — | — | E | — | — | — | P | — | — | — | Q | — | — | — | 0.06 | 2.07 | 0.07 | 986 |
| Phage-671 | — | — | D | — | — | E | L | R | — | M | A | — | — | — | 0.07 | 2.07 | 0.07 | 987 |
| Phage-672 | — | — | D | — | — | L | P | A | — | M | A | — | — | — | 0.07 | 2.06 | 0.12 | 988 |
| Phage-673 | — | — | E | — | — | S | D | E | — | M | L | — | — | — | 0.07 | 2.06 | 0.07 | 989 |
| Phage-674 | — | — | E | — | — | — | P | E | — | M | M | — | — | I | 0.05 | 2.06 | 0.06 | 990 |
| Phage-675 | — | — | E | — | — | — | D | I | — | M | Q | — | — | — | 0.06 | 2.06 | 0.07 | 991 |
| Phage-676 | — | — | E | — | — | S | A | E | — | M | — | — | — | — | 0.06 | 2.06 | 0.07 | 992 |
| Phage-677 | — | — | D | — | — | — | P | — | — | M | G | — | — | — | 0.08 | 2.06 | 0.08 | 993 |
| Phage-678 | — | — | E | — | — | L | P | I | — | M | D | — | — | — | 0.07 | 2.06 | 0.08 | 994 |
| Phage-679 | — | — | D | — | — | — | I | E | — | M | Q | — | — | — | 0.08 | 2.05 | 0.07 | 995 |
| Phage-680 | — | — | — | — | — | I | E | E | — | M | W | — | — | — | 0.06 | 2.05 | 0.06 | 996 |
| Phage-681 | — | — | Q | — | — | M | P | E | — | M | D | — | — | — | 0.08 | 2.05 | 0.07 | 997 |
| Phage-682 | — | — | D | — | — | — | P | E | — | V | M | — | — | — | 0.08 | 2.05 | 0.10 | 998 |
| Phage-683 | — | — | D | — | — | V | G | E | — | M | Q | — | — | — | 0.10 | 2.05 | 0.08 | 999 |
| Phage-684 | — | — | D | — | — | S | A | — | — | M | H | — | — | — | 0.06 | 2.05 | 0.06 | 1000 |
| Phage-685 | — | — | E | — | — | — | P | E | — | M | Y | — | — | — | 0.05 | 2.05 | 0.07 | 1001 |
| Phage-686 | — | — | D | — | — | L | P | E | — | Q | M | — | — | — | 0.06 | 2.05 | 0.08 | 1002 |
| Phage-687 | — | — | D | — | — | — | S | E | — | M | M | — | — | — | 0.06 | 2.05 | 0.06 | 1003 |
| Phage-688 | — | — | D | — | — | L | I | E | — | M | E | — | — | — | 0.06 | 2.04 | 0.06 | 1004 |
| Phage-689 | — | — | E | — | — | — | I | — | — | M | S | — | — | — | 0.06 | 2.04 | 0.06 | 1005 |
| Phage-690 | — | — | E | — | — | — | P | — | H | M | D | — | — | — | 0.06 | 2.04 | 0.08 | 1006 |
| Phage-691 | — | — | E | — | — | S | P | — | — | M | S | — | — | — | 0.06 | 2.04 | 0.07 | 1007 |
| Phage-692 | — | — | D | — | — | F | R | E | — | M | Q | — | — | — | 0.08 | 2.04 | 0.06 | 1008 |
| Phage-693 | — | — | L | — | — | — | P | — | — | — | Q | — | — | — | 0.08 | 2.04 | 0.14 | 1009 |
| Phage-694 | — | — | D | — | — | S | S | E | — | M | W | — | — | — | 0.06 | 2.03 | 0.06 | 1010 |
| Phage-695 | — | — | S | — | — | L | P | E | — | M | S | — | — | — | 0.06 | 2.03 | 0.09 | 1011 |
| Phage-696 | — | — | E | — | — | — | P | — | — | — | A | — | — | — | 0.06 | 2.03 | 0.08 | 1012 |
| Phage-697 | — | — | D | — | — | S | P | M | — | — | G | — | — | — | 0.08 | 2.03 | 0.07 | 1013 |
| Phage-698 | — | — | — | — | — | L | P | E | — | M | M | — | — | — | 0.05 | 2.03 | 0.11 | 1014 |
| Phage-699 | — | — | E | — | — | R | P | E | — | M | Q | — | — | — | 0.06 | 2.03 | 0.11 | 1015 |
| Phage-700 | — | — | E | — | — | — | R | E | H | M | A | — | — | — | 0.07 | 2.02 | 0.08 | 1016 |
| Phage-701 | — | — | E | — | — | S | P | E | — | M | T | — | — | — | 0.09 | 2.02 | 0.08 | 1017 |
| Phage-702 | — | — | E | — | — | S | I | — | — | M | A | — | — | — | 0.08 | 2.02 | 0.06 | 1018 |
| Phage-703 | — | — | E | — | — | S | L | E | — | M | G | — | — | — | 0.07 | 2.02 | 0.09 | 1019 |
| Phage-704 | — | — | D | — | — | — | P | E | — | M | E | — | — | — | 0.08 | 2.01 | 0.09 | 1020 |
| Phage-705 | — | — | D | — | — | M | G | V | — | Y | W | — | — | — | 0.07 | 2.01 | 0.09 | 1021 |
| Phage-706 | — | — | D | — | — | E | A | — | — | M | S | — | — | — | 0.08 | 2.01 | 0.09 | 1022 |
| Phage-707 | — | — | D | — | — | S | H | E | — | M | E | — | — | — | 0.05 | 2.01 | 0.06 | 1023 |
| Phage-708 | — | — | Q | — | — | L | P | E | — | M | S | — | — | — | 0.06 | 2.01 | 0.06 | 1024 |
| Phage-709 | — | — | E | — | — | L | S | — | — | M | Q | — | — | — | 0.11 | 2.00 | 0.09 | 1025 |
| Phage-710 | — | — | D | — | — | S | P | I | — | M | S | — | — | — | 0.06 | 2.00 | 0.09 | 1026 |
| Phage-711 | — | — | D | — | — | S | D | — | — | M | W | — | — | — | 0.09 | 2.00 | 0.11 | 1027 |
| Phage-712 | — | — | D | — | — | L | P | — | — | M | S | — | — | — | 0.07 | 2.00 | 0.10 | 1028 |
| Phage-713 | — | — | E | — | — | — | A | E | — | M | M | — | — | — | 0.05 | 1.99 | 0.07 | 1029 |
| Phage-714 | — | — | — | — | — | L | P | M | — | M | H | — | — | — | 0.13 | 1.99 | 0.18 | 1030 |
| Phage-715 | — | — | D | — | — | S | Q | E | — | — | A | — | — | — | 0.07 | 1.99 | 0.07 | 1031 |
| Phage-716 | — | — | D | — | — | M | P | — | — | M | S | — | — | — | 0.06 | 1.98 | 0.08 | 1032 |
| Phage-717 | — | — | V | — | — | L | P | — | — | M | E | — | — | — | 0.06 | 1.98 | 0.08 | 1033 |
| Phage-718 | — | — | D | — | — | L | G | I | — | M | T | — | — | — | 0.06 | 1.98 | 0.08 | 1034 |
| Phage-719 | — | — | D | — | — | L | — | E | — | M | D | — | — | — | 0.05 | 1.97 | 0.06 | 1035 |
| Phage-720 | — | — | E | — | — | L | P | M | — | F | L | — | — | M | 0.06 | 1.97 | 0.06 | 1036 |
| Phage-721 | — | — | D | — | — | L | P | I | — | M | T | Q | — | — | 0.06 | 1.97 | 0.08 | 1037 |
| Phage-722 | — | — | E | — | — | L | A | E | — | M | D | — | — | — | 0.07 | 1.96 | 0.07 | 1038 |
| Phage-723 | — | — | D | — | — | L | E | — | — | R | A | — | — | — | 0.06 | 1.96 | 0.08 | 1039 |
| Phage-724 | — | — | D | — | — | L | P | E | — | L | G | — | — | — | 0.07 | 1.96 | 0.08 | 1040 |
| Phage-725 | — | — | — | — | — | S | V | E | — | M | A | — | — | — | 0.07 | 1.96 | 0.08 | 1041 |
| Phage-726 | — | — | D | — | — | S | A | L | — | M | W | — | — | — | 0.11 | 1.96 | 0.08 | 1042 |
| Phage-727 | — | — | D | — | — | S | S | E | — | M | I | — | — | — | 0.06 | 1.95 | 0.06 | 1043 |
| Phage-728 | — | — | D | — | — | L | I | E | — | M | Y | — | — | — | 0.08 | 1.95 | 0.09 | 1044 |
| Phage-729 | — | — | D | — | — | S | A | E | — | M | Q | — | — | — | 0.06 | 1.95 | 0.07 | 1045 |
| Phage-730 | — | — | D | — | — | L | V | E | — | M | S | — | — | — | 0.09 | 1.95 | 0.08 | 1046 |
| Phage-731 | — | — | E | — | — | L | S | — | — | M | E | — | — | — | 0.06 | 1.95 | 0.08 | 1047 |
| Phage-732 | — | — | D | — | — | S | P | E | H | — | R | — | — | — | 0.08 | 1.94 | 0.11 | 1048 |
| Phage-733 | — | — | — | — | — | L | E | I | — | M | — | — | — | — | 0.05 | 1.94 | 0.07 | 1049 |
| Phage-734 | — | — | E | — | — | L | S | E | — | M | A | — | — | — | 0.06 | 1.94 | 0.20 | 1050 |
| Phage-735 | — | — | E | — | — | L | P | I | — | L | S | — | — | — | 0.07 | 1.94 | 0.08 | 1051 |
| Phage-736 | — | — | D | — | — | — | S | E | — | M | W | — | — | — | 0.06 | 1.94 | 0.06 | 1052 |
| Phage-737 | — | — | S | — | — | — | P | E | — | M | A | — | — | — | 0.05 | 1.94 | 0.06 | 1053 |
| Phage-738 | — | — | E | — | — | R | A | E | — | M | E | — | — | — | 0.06 | 1.93 | 0.06 | 1054 |
| Phage-739 | — | — | D | — | — | S | P | D | — | — | W | — | — | — | 0.07 | 1.93 | 0.10 | 1055 |
| Phage-740 | — | — | M | — | — | — | P | E | — | M | E | — | — | — | 0.06 | 1.93 | 0.07 | 1056 |
| Phage-741 | — | — | M | — | — | Q | P | E | — | M | Q | — | — | — | 0.08 | 1.93 | 0.08 | 1057 |
| Phage-742 | — | — | D | — | — | M | P | E | — | M | E | — | — | — | 0.08 | 1.93 | 0.09 | 1058 |
| Phage-743 | — | — | D | — | — | S | A | E | — | L | A | — | — | — | 0.07 | 1.93 | 0.08 | 1059 |
| Phage-744 | — | — | D | — | — | S | P | E | — | M | T | — | — | — | 0.06 | 1.93 | 0.07 | 1060 |
| Phage-745 | — | — | E | — | — | S | M | E | — | M | A | — | — | — | 0.07 | 1.92 | 0.07 | 1061 |
| Phage-746 | — | — | — | — | — | M | P | E | — | — | D | — | — | — | 0.05 | 1.92 | 0.06 | 1062 |
| Phage-747 | — | — | — | — | — | S | P | E | F | M | E | — | — | — | 0.06 | 1.91 | 0.06 | 1063 |
| Phage-748 | — | — | D | — | — | L | N | E | — | M | E | — | — | — | 0.05 | 1.91 | 0.06 | 1064 |
| Phage-749 | — | — | D | — | — | L | K | E | — | Q | E | — | — | — | 0.07 | 1.91 | 0.09 | 1065 |
| Phage-750 | — | — | E | — | — | L | A | — | — | M | W | — | — | — | 0.08 | 1.91 | 0.12 | 1066 |
| Phage-751 | — | — | E | — | — | L | A | V | — | M | Q | — | — | — | 0.08 | 1.91 | 0.10 | 1067 |
| Phage-752 | — | — | E | — | — | S | S | E | — | M | E | — | — | — | 0.05 | 1.91 | 0.06 | 1068 |
| Phage-753 | — | — | D | — | — | L | D | I | — | L | A | — | — | — | 0.07 | 1.91 | 0.08 | 1069 |
| Phage-754 | — | — | E | — | — | S | E | E | — | M | S | — | — | — | 0.06 | 1.91 | 0.07 | 1070 |
| Phage-755 | — | — | D | — | — | S | Q | E | — | M | W | — | — | — | 0.06 | 1.90 | 0.09 | 1071 |
| Phage-756 | — | — | D | — | — | — | P | E | — | M | K | — | — | — | 0.05 | 1.90 | 0.06 | 1072 |
| Phage-757 | — | — | D | — | — | S | K | E | — | M | H | — | — | — | 0.07 | 1.90 | 0.07 | 1073 |
| Phage-758 | — | — | D | — | — | E | P | — | — | L | E | — | — | — | 0.08 | 1.90 | 0.08 | 1074 |
| Phage-759 | — | — | D | — | — | — | E | E | — | M | S | — | — | — | 0.05 | 1.90 | 0.09 | 1075 |
| Phage-760 | — | — | D | — | — | V | E | E | — | L | Y | — | — | — | 0.05 | 1.90 | 0.06 | 1076 |
| Phage-761 | — | — | — | — | — | L | E | E | — | M | D | — | — | — | 0.05 | 1.90 | 0.06 | 1077 |
| Phage-762 | — | — | — | — | — | — | P | — | — | M | E | — | — | — | 0.08 | 1.90 | 0.08 | 1078 |
| Phage-763 | — | — | — | — | — | L | P | — | — | — | A | — | — | — | 0.07 | 1.90 | 0.09 | 1079 |
| Phage-764 | — | — | D | — | — | L | K | E | — | M | A | — | — | — | 0.05 | 1.90 | 0.07 | 1080 |
| Phage-765 | — | — | D | — | — | R | P | E | — | M | E | — | — | — | 0.05 | 1.90 | 0.06 | 1081 |
| Phage-766 | — | — | D | — | — | L | P | E | — | M | G | — | — | — | 0.06 | 1.89 | 0.11 | 1082 |
| Phage-767 | — | — | E | — | — | L | Q | E | — | M | E | — | — | — | 0.06 | 1.89 | 0.10 | 1083 |
| Phage-768 | — | — | E | — | — | I | P | E | — | M | A | — | — | — | 0.06 | 1.89 | 0.06 | 1084 |
| Phage-769 | — | — | D | — | — | S | P | E | — | M | P | — | — | — | 0.06 | 1.89 | 0.08 | 1085 |
| Phage-770 | — | — | D | — | — | L | A | E | — | M | P | — | — | — | 0.08 | 1.89 | 0.07 | 1086 |
| Phage-771 | — | — | E | — | — | S | P | E | — | M | W | — | — | — | 0.05 | 1.89 | 0.09 | 1087 |
| Phage-772 | — | — | D | — | — | I | D | E | — | L | Q | — | — | — | 0.06 | 1.88 | 0.06 | 1088 |
| Phage-773 | — | — | E | — | — | V | A | E | — | M | L | — | — | — | 0.05 | 1.88 | 0.05 | 1089 |
| Phage-774 | — | — | E | — | — | L | S | E | — | M | S | — | — | — | 0.07 | 1.88 | 0.09 | 1090 |
| Phage-775 | — | — | E | — | — | M | S | E | — | M | A | — | — | — | 0.08 | 1.88 | 0.11 | 1091 |
| Phage-776 | — | — | D | — | — | S | — | E | — | —— | Q | — | — | — | 0.06 | 1.88 | 0.07 | 1092 |
| Phage-777 | — | — | E | — | — | S | L | E | — | M | A | — | — | — | 0.07 | 1.88 | 0.08 | 1093 |
| Phage-778 | — | — | E | — | — | S | Y | E | — | M | W | — | — | — | 0.06 | 1.88 | 0.08 | 1094 |
| Phage-779 | — | — | E | — | — | L | P | E | — | M | M | — | — | — | 0.06 | 1.88 | 0.27 | 1095 |
| Phage-780 | — | — | D | — | — | L | P | E | — | M | R | — | — | — | 0.07 | 1.88 | 0.08 | 1096 |
| Phage-781 | — | — | E | — | — | S | P | L | — | M | S | — | — | — | 0.06 | 1.87 | 0.09 | 1097 |
| Phage-782 | — | — | E | — | — | S | — | E | — | — | G | — | — | — | 0.08 | 1.87 | 0.07 | 1098 |
| Phage-783 | — | — | D | — | — | S | A | L | — | M | S | — | — | — | 0.06 | 1.87 | 0.08 | 1099 |
| Phage-784 | — | — | D | — | — | T | G | V | — | F | Y | — | — | — | 0.07 | 1.87 | 0.08 | 1100 |
| Phage-785 | — | — | E | — | — | T | S | E | — | M | — | — | — | — | 0.06 | 1.87 | 0.06 | 1101 |
| Phage-786 | — | — | E | — | — | R | S | E | — | M | A | — | — | — | 0.06 | 1.86 | 0.06 | 1102 |
| Phage-787 | — | — | D | — | — | L | A | M | — | M | D | — | — | — | 0.05 | 1.86 | 0.08 | 1103 |
| Phage-788 | — | — | D | — | — | M | P | E | — | L | A | — | — | — | 0.06 | 1.86 | 0.07 | 1104 |
| Phage-789 | — | — | E | — | — | — | A | E | — | M | S | — | — | — | 0.08 | 1.86 | 0.07 | 1105 |
| Phage-790 | — | — | — | — | — | S | S | E | — | M | W | — | — | — | 0.08 | 1.85 | 0.07 | 1106 |
| Phage-791 | — | — | D | — | — | S | — | A | — | M | W | — | — | — | 0.11 | 1.85 | 0.13 | 1107 |
| Phage-792 | — | — | D | — | — | S | A | A | — | — | M | — | — | — | 0.06 | 1.84 | 0.09 | 1108 |
| Phage-793 | — | — | D | — | — | S | L | — | — | L | G | — | — | — | 0.06 | 1.84 | 0.09 | 1109 |
| Phage-794 | — | — | E | — | — | S | Q | E | — | M | Q | — | — | — | 0.05 | 1.84 | 0.05 | 1110 |
| Phage-795 | — | — | — | — | — | L | P | E | — | L | S | — | — | — | 0.07 | 1.84 | 0.07 | 1111 |
| Phage-796 | — | — | E | — | — | L | Q | I | — | M | S | — | — | — | 0.10 | 1.84 | 0.14 | 1112 |
| Phage-797 | — | — | — | — | — | — | P | E | — | M | F | — | — | M | 0.06 | 1.83 | 0.06 | 1113 |
| Phage-798 | — | — | E | — | — | R | P | E | — | M | A | — | — | — | 0.06 | 1.83 | 0.07 | 1114 |
| Phage-799 | — | — | E | — | — | L | P | E | — | M | R | — | — | — | 0.05 | 1.83 | 0.06 | 1115 |
| Phage-800 | — | — | D | — | — | S | P | A | — | M | A | — | — | — | 0.09 | 1.83 | 0.10 | 1116 |
| Phage-801 | — | — | D | — | — | L | P | M | — | F | A | — | — | M | 0.06 | 1.83 | 0.06 | 1117 |
| Phage-802 | — | — | D | — | — | S | S | E | — | — | V | — | — | — | 0.08 | 1.83 | 0.06 | 1118 |
| Phage-803 | — | — | E | — | — | — | A | E | — | M | W | — | — | — | 0.09 | 1.83 | 0.07 | 1119 |
| Phage-804 | — | — | E | — | — | S | A | E | — | — | F | — | — | — | 0.07 | 1.83 | 0.05 | 1120 |
| Phage-805 | — | — | E | — | — | S | P | M | — | M | Q | — | — | M | 0.07 | 1.83 | 0.06 | 1121 |
| Phage-806 | — | — | D | — | — | S | A | E | — | M | E | — | — | I | 0.06 | 1.83 | 0.07 | 1122 |
| Phage-807 | — | — | D | — | — | L | P | L | — | M | H | — | — | — | 0.05 | 1.82 | 0.07 | 1123 |
| Phage-808 | — | — | E | — | — | — | P | — | — | L | — | — | — | — | 0.09 | 1.82 | 0.08 | 1124 |
| Phage-809 | — | — | D | — | — | — | R | E | — | M | T | — | — | — | 0.06 | 1.82 | 0.06 | 1125 |
| Phage-810 | — | — | D | — | — | T | E | E | — | M | D | — | — | — | 0.07 | 1.82 | 0.09 | 1126 |
| Phage-811 | — | — | D | — | — | S | S | M | — | M | A | — | — | — | 0.05 | 1.82 | 0.06 | 1127 |
| Phage-812 | — | — | — | — | — | S | S | E | — | — | W | — | — | — | 0.06 | 1.82 | 0.06 | 1128 |
| Phage-813 | — | — | E | — | — | S | A | E | F | M | T | — | — | — | 0.06 | 1.81 | 0.06 | 1129 |
| Phage-814 | — | — | Q | — | — | S | A | E | — | M | M | — | — | — | 0.06 | 1.81 | 0.07 | 1130 |
| Phage-815 | — | — | — | — | — | L | P | — | — | M | — | — | — | — | 0.05 | 1.81 | 0.06 | 1131 |
| Phage-816 | — | — | D | — | — | K | A | E | — | M | E | — | — | — | 0.11 | 1.81 | 0.09 | 1132 |
| Phage-817 | — | — | — | — | — | L | P | M | — | M | A | — | — | — | 0.05 | 1.80 | 0.08 | 1133 |
| Phage-818 | — | — | D | — | — | T | R | E | — | — | D | — | — | — | 0.06 | 1.80 | 0.08 | 1134 |
| Phage-819 | — | — | V | — | — | — | P | E | F | M | A | — | — | — | 0.07 | 1.80 | 0.10 | 1135 |
| Phage-820 | — | — | — | — | — | — | E | — | — | M | A | — | — | — | 0.07 | 1.80 | 0.08 | 1136 |
| Phage-821 | — | — | D | — | — | L | S | E | — | M | F | — | — | — | 0.05 | 1.79 | 0.10 | 1137 |
| Phage-822 | — | — | D | — | — | L | G | V | — | M | A | — | — | — | 0.05 | 1.79 | 0.07 | 1138 |
| Phage-823 | — | — | D | — | — | T | S | E | — | — | T | — | — | — | 0.07 | 1.79 | 0.07 | 1139 |
| Phage-824 | — | — | D | — | — | S | P | E | — | — | A | — | — | — | 0.08 | 1.79 | 0.23 | 1140 |
| Phage-825 | — | — | D | — | — | L | A | E | — | M | V | — | — | — | 0.08 | 1.79 | 0.08 | 1141 |
| Phage-826 | — | — | D | — | — | — | P | M | — | M | A | — | — | — | 0.06 | 1.79 | 0.08 | 1142 |
| Phage-827 | — | — | E | — | — | L | P | A | — | M | Q | — | — | — | 0.06 | 1.78 | 0.07 | 1143 |
| Phage-828 | — | — | E | — | — | H | P | — | — | M | A | — | — | — | 0.06 | 1.78 | 0.09 | 1144 |
| Phage-829 | — | — | — | — | — | V | P | E | — | M | A | — | — | — | 0.06 | 1.78 | 0.07 | 1145 |
| Phage-830 | — | — | D | — | — | S | A | E | — | — | T | — | — | — | 0.06 | 1.77 | 0.06 | 1146 |
| Phage-831 | — | — | E | — | — | L | D | E | — | M | W | — | — | — | 0.06 | 1.77 | 0.06 | 1147 |
| Phage-832 | — | — | D | — | L | — | S | E | — | M | S | — | — | — | 0.05 | 1.77 | 0.05 | 1148 |
| Phage-833 | — | — | D | — | — | S | S | E | — | — | A | — | — | — | 0.09 | 1.77 | 0.07 | 1149 |
| Phage-834 | — | — | E | — | — | S | P | — | — | V | Q | — | — | — | 0.05 | 1.77 | 0.06 | 1150 |
| Phage-835 | — | — | E | — | — | S | P | E | — | M | R | — | — | — | 0.07 | 1.77 | 0.08 | 1151 |
| Phage-836 | — | — | D | — | — | L | D | E | — | M | E | — | — | — | 0.06 | 1.76 | 0.08 | 1152 |
| Phage-837 | — | — | — | — | — | L | P | E | — | M | L | — | — | — | 0.07 | 1.76 | 0.09 | 1153 |
| Phage-838 | — | — | E | — | — | L | P | I | — | M | S | — | — | — | 0.07 | 1.76 | 0.09 | 1154 |
| Phage-839 | — | — | V | — | — | L | Q | E | — | M | E | — | — | — | 0.06 | 1.76 | 0.06 | 1155 |
| Phage-840 | — | — | D | — | — | S | P | — | — | M | Q | — | — | — | 0.06 | 1.76 | 0.07 | 1156 |
| Phage-841 | — | — | D | — | — | I | P | E | — | L | A | — | — | — | 0.05 | 1.76 | 0.06 | 1157 |
| Phage-842 | — | — | D | — | — | S | Q | E | — | M | L | — | — | — | 0.05 | 1.76 | 0.05 | 1158 |
| Phage-843 | — | — | D | — | — | V | E | P | — | L | S | — | — | — | 0.07 | 1.76 | 0.10 | 1159 |
| Phage-844 | — | — | D | — | — | K | P | — | — | M | E | — | — | — | 0.06 | 1.76 | 0.08 | 1160 |
| Phage-845 | — | — | D | — | — | I | G | E | — | M | V | — | — | — | 0.06 | 1.76 | 0.66 | 1161 |
| Phage-846 | — | — | E | — | — | L | A | — | — | M | — | — | — | — | 0.08 | 1.76 | 0.07 | 1162 |
| Phage-847 | — | — | D | — | — | V | Q | E | — | M | Q | — | — | — | 0.06 | 1.76 | 0.07 | 1163 |
| Phage-848 | — | — | H | — | — | L | Q | E | — | M | A | — | — | — | 0.08 | 1.76 | 0.07 | 1164 |
| Phage-849 | — | — | D | — | — | S | P | E | — | M | — | — | — | — | 0.06 | 1.75 | 0.06 | 1165 |
| Phage-850 | — | — | D | — | — | — | P | V | — | M | A | — | — | — | 0.06 | 1.75 | 0.06 | 1166 |
| Phage-851 | — | — | E | — | — | S | A | E | — | M | L | — | — | — | 0.07 | 1.75 | 0.07 | 1167 |
| Phage-852 | — | — | D | — | — | H | I | E | — | L | A | — | — | — | 0.07 | 1.75 | 0.10 | 1168 |
| Phage-853 | — | — | S | — | — | L | P | E | — | M | Q | — | — | — | 0.06 | 1.75 | 0.08 | 1169 |
| Phage-854 | — | — | E | — | — | L | P | E | — | M | Q | — | — | — | 0.07 | 1.75 | 0.07 | 1170 |
| Phage-855 | — | — | D | — | — | S | L | — | — | M | D | — | — | — | 0.06 | 1.75 | 0.06 | 1171 |
| Phage-856 | — | — | D | — | — | Y | P | E | — | M | S | — | — | — | 0.07 | 1.75 | 0.06 | 1172 |
| Phage-857 | — | — | D | — | — | — | S | E | — | M | A | — | — | — | 0.06 | 1.75 | 0.09 | 1173 |
| Phage-858 | — | — | D | — | — | — | E | K | — | M | Q | — | — | — | 0.06 | 1.74 | 0.06 | 1174 |
| Phage-859 | — | — | E | — | — | — | I | E | — | L | A | — | — | — | 0.05 | 1.74 | 0.07 | 1175 |
| Phage-860 | — | — | E | — | — | S | A | E | — | M | E | — | — | — | 0.06 | 1.73 | 0.07 | 1176 |
| Phage-861 | — | — | S | — | — | S | A | E | — | M | L | — | — | — | 0.07 | 1.73 | 0.08 | 1177 |
| Phage-862 | — | — | E | — | — | L | P | D | — | M | Q | — | — | — | 0.06 | 1.73 | 0.07 | 1178 |
| Phage-863 | — | — | D | — | — | L | E | E | — | M | V | — | — | M | 0.06 | 1.73 | 0.06 | 1179 |
| Phage-864 | — | — | E | — | — | L | Q | T | — | M | A | — | — | — | 0.08 | 1.73 | 0.07 | 1180 |
| Phage-865 | — | — | — | — | — | S | Q | E | H | — | Q | — | — | — | 0.07 | 1.73 | 0.09 | 1181 |
| Phage-866 | — | — | D | — | — | L | P | E | — | — | F | — | — | — | 0.05 | 1.73 | 0.09 | 1182 |
| Phage-867 | — | — | S | — | — | L | P | E | — | M | G | — | — | — | 0.06 | 1.73 | 0.10 | 1183 |
| Phage-868 | — | — | D | — | — | — | E | E | — | L | W | — | — | — | 0.05 | 1.73 | 0.06 | 1184 |
| Phage-869 | — | — | — | — | — | S | A | E | — | M | A | — | — | — | 0.05 | 1.73 | 0.07 | 1185 |
| Phage-870 | — | — | D | — | — | S | G | V | — | M | W | — | — | — | 0.08 | 1.73 | 0.08 | 1186 |
| Phage-871 | — | — | D | — | — | T | Q | A | — | — | Q | — | — | — | 0.05 | 1.73 | 0.06 | 1187 |
| Phage-872 | — | — | D | — | — | — | I | — | — | — | G | — | — | — | 0.06 | 1.72 | 0.09 | 1188 |
| Phage-873 | — | — | D | — | — | — | A | E | F | M | M | — | — | — | 0.05 | 1.72 | 0.06 | 1189 |
| Phage-874 | — | — | D | — | — | L | S | E | — | M | A | — | — | — | 0.07 | 1.72 | 0.08 | 1190 |
| Phage-875 | — | — | E | — | — | S | E | M | — | M | K | — | — | — | 0.08 | 1.72 | 0.07 | 1191 |
| Phage-876 | — | — | D | — | — | L | Q | I | — | L | W | — | — | — | 0.06 | 1.72 | 0.07 | 1192 |
| Phage-877 | — | — | Q | — | — | L | P | V | — | M | E | — | — | — | 0.06 | 1.72 | 0.06 | 1193 |
| Phage-878 | — | — | D | — | — | — | A | E | — | M | D | — | — | — | 0.05 | 1.72 | 0.07 | 1194 |
| Phage-879 | — | — | E | — | — | S | V | — | — | M | Q | — | — | — | 0.06 | 1.71 | 0.06 | 1195 |
| Phage-880 | — | — | — | — | — | L | P | E | — | L | D | — | — | — | 0.06 | 1.71 | 0.07 | 1196 |
| Phage-881 | — | — | D | — | — | S | R | E | — | M | V | — | — | — | 0.06 | 1.71 | 0.06 | 1197 |
| Phage-882 | — | — | D | — | — | L | V | E | — | R | G | — | — | — | 0.06 | 1.71 | 0.07 | 1198 |
| Phage-883 | — | — | — | — | — | S | A | E | — | M | W | — | — | — | 0.06 | 1.71 | 0.11 | 1199 |
| Phage-884 | — | — | D | — | — | L | Q | I | — | M | H | — | — | — | 0.07 | 1.71 | 0.10 | 1200 |
| Phage-885 | — | — | — | — | — | S | M | E | — | M | A | — | — | — | 0.06 | 1.71 | 0.07 | 1201 |
| Phage-886 | — | — | D | — | — | L | P | E | — | F | L | — | — | — | 0.09 | 1.71 | 0.11 | 1202 |
| Phage-887 | — | — | D | — | — | S | L | A | — | — | A | — | — | — | 0.05 | 1.70 | 0.06 | 1203 |
| Phage-888 | — | — | — | — | — | T | Q | E | — | M | Y | — | — | — | 0.06 | 1.70 | 0.05 | 1204 |
| Phage-889 | — | — | E | — | — | — | Q | E | — | M | S | — | — | — | 0.05 | 1.70 | 0.08 | 1205 |
| Phage-890 | — | — | E | — | — | L | P | — | — | F | M | — | — | — | 0.06 | 1.70 | 0.07 | 1206 |
| Phage-891 | — | — | D | — | — | S | — | E | — | M | E | — | — | — | 0.05 | 1.70 | 0.12 | 1207 |
| Phage-892 | — | — | — | — | — | S | P | L | — | M | L | — | — | — | 0.06 | 1.70 | 0.10 | 1208 |
| Phage-893 | — | — | E | — | — | S | A | E | — | M | Q | — | — | — | 0.08 | 1.70 | 0.29 | 1209 |
| Phage-894 | — | — | D | — | — | L | P | V | — | L | E | — | — | — | 0.05 | 1.69 | 0.06 | 1210 |
| Phage-895 | — | — | — | — | — | L | Q | E | — | M | Q | — | — | — | 0.06 | 1.69 | 0.07 | 1211 |
| Phage-896 | — | — | D | — | — | — | I | E | — | M | A | — | — | — | 0.17 | 1.69 | 0.08 | 1212 |
| Phage-897 | — | — | E | — | — | K | P | E | — | M | Q | — | — | — | 0.06 | 1.69 | 0.06 | 1213 |
| Phage-898 | — | — | D | — | — | — | — | E | — | M | T | — | — | — | 0.10 | 1.69 | 0.35 | 1214 |
| Phage-899 | — | — | E | — | — | S | P | W | — | M | — | — | — | — | 0.06 | 1.68 | 0.06 | 1215 |
| Phage-900 | — | — | D | — | — | S | P | A | — | — | W | — | — | — | 0.08 | 1.68 | 0.08 | 1216 |
| Phage-901 | — | — | D | — | — | S | E | — | — | M | S | — | — | — | 0.06 | 1.68 | 0.07 | 1217 |
| Phage-902 | — | — | D | — | — | — | K | E | — | M | D | — | — | — | 0.08 | 1.68 | 0.08 | 1218 |
| Phage-903 | — | — | D | — | — | S | T | E | — | M | E | — | — | — | 0.06 | 1.68 | 0.08 | 1219 |
| Phage-904 | — | — | D | — | — | S | S | L | — | M | H | — | — | — | 0.06 | 1.67 | 0.07 | 1220 |
| Phage-905 | — | — | D | — | — | S | — | — | — | — | S | — | — | — | 0.07 | 1.67 | 0.05 | 1221 |
| Phage-906 | — | — | E | — | — | — | P | — | — | M | — | — | — | — | 0.08 | 1.67 | 0.08 | 1222 |
| Phage-907 | — | — | D | — | — | — | P | — | W | M | A | — | — | — | 0.08 | 1.67 | 0.10 | 1223 |
| Phage-908 | — | — | D | — | — | S | R | E | — | M | S | — | — | — | 0.07 | 1.67 | 0.10 | 1224 |
| Phage-909 | — | — | D | — | — | — | A | E | — | M | A | — | — | V | 0.06 | 1.67 | 0.07 | 1225 |
| Phage-910 | — | — | E | — | — | — | Q | E | — | M | — | — | — | — | 0.06 | 1.67 | 0.06 | 1226 |
| Phage-911 | — | — | E | — | — | — | P | — | — | M | T | — | — | — | 0.06 | 1.67 | 0.08 | 1227 |
| Phage-912 | — | — | D | — | — | S | A | D | — | M | A | — | — | — | 0.06 | 1.67 | 0.07 | 1228 |
| Phage-913 | — | — | D | — | — | I | G | L | — | L | — | — | — | — | 0.07 | 1.66 | 0.06 | 1229 |
| Phage-914 | — | — | E | — | — | L | P | M | — | M | I | — | — | — | 0.07 | 1.66 | 0.09 | 1230 |
| Phage-915 | — | — | E | — | — | L | P | — | — | M | R | P | — | — | 0.06 | 1.66 | 0.08 | 1231 |
| Phage-916 | — | — | E | — | — | L | S | — | — | M | S | — | — | — | 0.05 | 1.66 | 0.07 | 1232 |
| Phage-917 | — | — | D | — | — | S | P | E | — | — | G | — | — | — | 0.06 | 1.66 | 0.07 | 1233 |
| Phage-918 | — | — | — | — | — | L | P | E | F | M | D | — | — | — | 0.06 | 1.66 | 0.06 | 1234 |
| Phage-919 | — | — | D | — | — | S | R | E | — | M | — | — | — | — | 0.05 | 1.66 | 0.06 | 1235 |
| Phage-920 | — | — | D | — | — | D | V | E | — | M | A | — | — | — | 0.07 | 1.66 | 0.07 | 1236 |
| Phage-921 | — | — | D | — | — | S | E | V | — | M | A | — | — | — | 0.07 | 1.66 | 0.06 | 1237 |
| Phage-922 | — | — | D | — | — | S | L | L | — | L | S | — | — | — | 0.07 | 1.65 | 0.07 | 1238 |
| Phage-923 | — | — | E | — | — | L | A | V | — | M | E | — | — | — | 0.08 | 1.65 | 0.12 | 1239 |
| Phage-924 | — | — | E | — | — | S | L | M | — | M | W | — | — | — | 0.06 | 1.65 | 0.08 | 1240 |
| Phage-925 | — | — | — | — | — | — | A | E | — | M | T | — | — | — | 0.06 | 1.65 | 0.07 | 1241 |
| Phage-926 | — | — | D | — | — | S | V | E | — | M | E | — | — | — | 0.07 | 1.65 | 0.09 | 1242 |
| Phage-927 | — | — | D | — | — | — | Q | E | — | R | L | — | — | — | 0.07 | 1.65 | 0.07 | 1243 |
| Phage-928 | — | — | D | — | — | S | A | E | — | L | L | — | — | — | 0.07 | 1.65 | 0.07 | 1244 |
| Phage-929 | — | — | D | — | — | G | P | — | — | — | Q | — | — | — | 0.06 | 1.64 | 0.07 | 1245 |
| Phage-930 | — | — | E | — | — | I | A | E | — | M | Q | — | — | — | 0.06 | 1.64 | 0.13 | 1246 |
| Phage-931 | — | — | E | — | — | S | A | — | — | M | H | — | — | — | 0.05 | 1.64 | 0.06 | 1247 |
| Phage-932 | — | — | E | — | — | T | A | E | H | M | Q | — | — | — | 0.05 | 1.64 | 0.05 | 1248 |
| Phage-933 | — | — | — | — | — | V | A | E | — | M | A | — | — | — | 0.06 | 1.64 | 0.06 | 1249 |
| Phage-934 | — | — | D | — | — | — | T | E | — | M | Q | — | — | — | 0.05 | 1.64 | 0.10 | 1250 |
| Phage-935 | — | — | E | — | — | M | — | E | — | M | A | — | — | — | 0.08 | 1.64 | 0.13 | 1251 |
| Phage-936 | — | — | D | — | — | D | V | E | — | M | S | — | — | — | 0.06 | 1.64 | 0.05 | 1252 |
| Phage-937 | — | — | — | — | — | S | L | E | — | M | Q | — | — | — | 0.06 | 1.63 | 0.07 | 1253 |
| Phage-938 | — | — | E | — | — | D | L | I | — | M | W | — | — | — | 0.06 | 1.63 | 0.06 | 1254 |
| Phage-939 | — | — | — | — | — | — | P | E | — | M | S | — | — | — | 0.05 | 1.63 | 0.05 | 1255 |
| Phage-940 | — | — | E | — | — | S | S | E | — | M | W | — | — | — | 0.07 | 1.63 | 0.09 | 1256 |
| Phage-941 | — | — | D | — | — | L | D | V | — | R | A | — | — | — | 0.07 | 1.63 | 0.09 | 1257 |
| Phage-942 | — | — | E | — | — | L | E | — | — | M | Q | — | — | — | 0.05 | 1.63 | 0.06 | 1258 |
| Phage-943 | — | — | E | — | — | S | — | V | — | M | A | — | — | — | 0.09 | 1.63 | 0.07 | 1259 |
| Phage-944 | — | — | D | — | — | S | L | E | — | — | G | — | — | — | 0.06 | 1.63 | 0.06 | 1260 |
| Phage-945 | — | — | D | — | — | S | E | A | F | M | Q | — | — | — | 0.07 | 1.63 | 0.07 | 1261 |
| Phage-946 | — | — | — | — | — | L | P | E | — | M | G | — | — | — | 0.05 | 1.63 | 0.07 | 1262 |
| Phage-947 | — | — | — | — | — | L | A | I | — | M | S | — | — | — | 0.08 | 1.63 | 0.08 | 1263 |
| Phage-948 | — | — | D | — | — | L | S | E | — | M | S | — | — | — | 0.05 | 1.62 | 0.06 | 1264 |
| Phage-949 | — | — | E | — | — | V | T | E | — | M | Q | — | — | — | 0.07 | 1.62 | 0.08 | 1265 |
| Phage-950 | — | — | D | — | — | — | Q | E | — | M | D | — | — | — | 0.06 | 1.62 | 0.08 | 1266 |
| Phage-951 | — | — | E | — | — | — | P | — | — | — | M | — | — | — | 0.06 | 1.62 | 0.06 | 1267 |
| Phage-952 | — | — | D | — | — | E | L | K | — | M | L | — | — | — | 0.05 | 1.62 | 0.08 | 1268 |
| Phage-953 | — | — | D | — | — | L | L | E | — | K | A | — | — | — | 0.07 | 1.62 | 0.07 | 1269 |
| Phage-954 | — | — | E | — | — | L | E | E | — | M | A | — | — | — | 0.08 | 1.62 | 0.07 | 1270 |
| Phage-955 | — | — | D | — | — | S | P | E | — | M | L | — | — | — | 0.07 | 1.61 | 0.07 | 1271 |
| Phage-956 | — | — | — | — | — | S | P | — | — | M | E | — | — | — | 0.06 | 1.61 | 0.06 | 1272 |
| Phage-957 | — | — | D | — | — | S | V | K | — | M | A | — | — | — | 0.07 | 1.61 | 0.06 | 1273 |
| Phage-958 | — | — | S | — | — | L | P | E | — | M | W | — | — | — | 0.07 | 1.61 | 0.10 | 1274 |
| Phage-959 | — | — | D | — | — | S | V | E | — | K | L | — | — | M | 0.05 | 1.61 | 0.06 | 1275 |
| Phage-960 | — | — | D | — | — | — | Q | E | — | M | V | — | — | — | 0.05 | 1.61 | 0.06 | 1276 |
| Phage-961 | — | — | — | — | — | S | P | E | — | M | Q | — | — | — | 0.06 | 1.60 | 0.08 | 1277 |
| Phage-962 | — | — | E | — | — | L | P | L | — | M | D | — | — | — | 0.07 | 1.60 | 0.08 | 1278 |
| Phage-963 | — | — | E | — | — | S | A | A | — | M | Q | — | — | — | 0.07 | 1.60 | 0.09 | 1279 |
| Phage-964 | — | — | E | — | — | M | P | E | — | L | Q | — | — | — | 0.05 | 1.60 | 0.10 | 1280 |
| Phage-965 | — | — | D | — | — | E | L | — | — | M | A | — | — | — | 0.07 | 1.60 | 0.08 | 1281 |
| Phage-966 | — | — | D | — | — | R | L | E | — | L | E | — | — | — | 0.06 | 1.60 | 0.06 | 1282 |
| Phage-967 | — | — | — | — | — | L | D | — | — | — | E | — | — | — | 0.06 | 1.60 | 0.10 | 1283 |
| Phage-968 | — | — | D | — | — | — | Q | E | F | — | W | — | — | — | 0.06 | 1.60 | 0.10 | 1284 |
| Phage-969 | — | — | D | — | — | S | E | A | — | M | W | — | — | — | 0.07 | 1.59 | 0.07 | 1285 |
| Phage-970 | — | — | E | — | — | — | D | E | — | M | W | — | — | — | 0.07 | 1.59 | 0.06 | 1286 |
| Phage-971 | — | — | E | — | — | S | V | E | — | — | G | — | — | — | 0.06 | 1.59 | 0.07 | 1287 |
| Phage-972 | — | — | D | — | — | R | V | E | — | M | A | — | — | — | 0.05 | 1.58 | 0.09 | 1288 |
| Phage-973 | — | — | E | — | — | S | — | E | H | M | E | — | — | — | 0.07 | 1.58 | 0.06 | 1289 |
| Phage-974 | — | — | — | — | — | S | S | E | — | — | P | — | — | — | 0.06 | 1.58 | 0.09 | 1290 |
| Phage-975 | — | — | D | — | — | L | P | — | — | L | Q | — | — | T | 0.07 | 1.58 | 0.07 | 1291 |
| Phage-976 | — | — | D | — | — | — | K | E | — | — | Q | — | — | — | 0.06 | 1.58 | 0.06 | 1292 |
| Phage-977 | — | — | E | — | — | L | P | — | — | M | V | — | — | — | 0.07 | 1.58 | 0.07 | 1293 |
| Phage-978 | — | — | Q | — | — | — | D | E | — | M | A | — | — | — | 0.07 | 1.58 | 0.08 | 1294 |
| Phage-979 | — | — | D | — | — | — | E | E | — | L | L | — | — | — | 0.05 | 1.57 | 0.05 | 1295 |
| Phage-980 | — | — | E | — | — | L | G | I | — | M | T | — | — | — | 0.06 | 1.57 | 0.08 | 1296 |
| Phage-981 | — | — | D | — | — | S | — | — | — | M | W | — | — | I | 0.06 | 1.57 | 0.09 | 1297 |
| Phage-982 | — | — | E | — | — | S | E | E | F | M | S | — | — | — | 0.05 | 1.57 | 0.08 | 1298 |
| Phage-983 | — | — | D | — | — | L | P | M | — | M | K | — | — | — | 0.08 | 1.57 | 0.10 | 1299 |
| Phage-984 | — | — | Q | — | — | L | P | — | — | V | E | — | — | — | 0.08 | 1.57 | 0.08 | 1300 |
| Phage-985 | — | — | E | — | — | L | D | M | — | V | W | — | — | — | 0.06 | 1.56 | 0.08 | 1301 |
| Phage-986 | — | — | D | — | — | S | Q | E | — | M | A | H | — | — | 0.06 | 1.56 | 0.07 | 1302 |
| Phage-987 | — | — | D | — | — | S | P | L | — | M | V | — | — | — | 0.05 | 1.56 | 0.06 | 1303 |
| Phage-988 | — | — | D | — | — | F | L | T | — | M | T | — | — | — | 0.06 | 1.56 | 0.07 | 1304 |
| Phage-989 | — | — | — | — | — | S | E | E | — | M | D | — | — | — | 0.05 | 1.55 | 0.05 | 1305 |
| Phage-990 | — | — | D | — | — | S | E | V | — | M | S | — | — | — | 0.06 | 1.55 | 0.06 | 1306 |
| Phage-991 | — | — | E | — | — | T | L | E | — | M | W | — | — | — | 0.07 | 1.55 | 0.33 | 1307 |
| Phage-992 | — | — | D | — | — | S | Q | A | H | M | Q | — | — | — | 0.06 | 1.55 | 0.08 | 1308 |
| Phage-993 | — | — | — | — | — | D | Q | — | — | M | E | — | — | — | 0.14 | 1.55 | 0.23 | 1309 |
| Phage-994 | — | — | D | — | — | L | P | V | — | M | M | — | — | — | 0.06 | 1.55 | 0.07 | 1310 |
| Phage-995 | — | — | — | — | — | N | A | — | — | M | S | — | — | — | 0.06 | 1.55 | 0.05 | 1311 |
| Phage-996 | — | — | D | — | — | T | S | E | — | M | T | — | — | — | 0.06 | 1.54 | 0.06 | 1312 |
| Phage-997 | — | — | E | — | — | — | P | V | — | M | F | — | — | — | 0.05 | 1.54 | 0.08 | 1313 |
| Phage-998 | — | — | E | — | — | L | G | — | — | M | E | — | — | — | 0.06 | 1.54 | 0.08 | 1314 |
| Phage-999 | — | — | D | — | — | N | G | V | — | W | W | — | — | I | 0.06 | 1.53 | 0.07 | 1315 |
| Phage-1000 | — | — | D | — | — | — | P | E | — | L | T | — | — | — | 0.08 | 1.53 | 0.06 | 1316 |
| Phage-1001 | — | — | E | — | — | S | I | — | — | M | T | — | — | — | 0.06 | 1.53 | 0.13 | 1317 |
| Phage-1002 | — | — | D | — | — | L | Q | I | — | M | G | — | — | — | 0.05 | 1.53 | 0.06 | 1318 |
| Phage-1003 | — | — | D | — | — | S | V | E | — | L | D | — | — | — | 0.05 | 1.53 | 0.08 | 1319 |
| Phage-1004 | — | — | D | — | — | — | — | — | — | M | W | — | — | — | 0.09 | 1.53 | 0.07 | 1320 |
| Phage-1005 | — | — | D | — | — | L | P | E | — | L | A | — | — | — | 0.05 | 1.52 | 0.06 | 1321 |
| Phage-1006 | — | — | D | — | — | L | A | V | — | M | A | — | — | — | 0.07 | 1.52 | 0.08 | 1322 |
| Phage-1007 | — | — | — | — | — | L | P | V | — | M | D | — | — | — | 0.09 | 1.52 | 0.08 | 1323 |
| Phage-1008 | — | — | E | — | — | S | A | M | — | — | W | — | — | — | 0.09 | 1.52 | 0.10 | 1324 |
| Phage-1009 | — | — | — | — | — | L | E | E | — | M | W | — | — | — | 0.07 | 1.51 | 0.08 | 1325 |
| Phage-1010 | — | — | D | — | — | S | E | L | — | M | A | — | — | — | 0.06 | 1.51 | 0.06 | 1326 |
| Phage-1011 | — | — | D | — | — | S | A | E | — | L | S | — | — | — | 0.07 | 1.51 | 0.07 | 1327 |
| Phage-1012 | — | — | E | — | — | M | P | — | — | M | E | — | — | — | 0.06 | 1.51 | 0.06 | 1328 |
| Phage-1013 | — | — | E | — | — | S | L | M | — | M | A | — | — | — | 0.06 | 1.50 | 0.06 | 1329 |
| Phage-1014 | — | — | D | — | — | S | Q | E | — | M | E | — | — | — | 0.06 | 1.50 | 0.06 | 1330 |
| Phage-1015 | — | — | D | — | — | K | A | E | — | M | Q | — | — | — | 0.09 | 1.50 | 0.06 | 1331 |
| Phage-1016 | — | — | D | — | — | S | P | — | — | — | Q | — | — | — | 0.07 | 1.50 | 0.07 | 1332 |
| Phage-1017 | — | — | S | — | — | L | P | — | — | L | E | — | — | — | 0.11 | 1.50 | 0.31 | 1333 |
| Phage-1018 | — | — | D | — | — | H | K | E | — | L | L | — | — | — | 0.05 | 1.49 | 0.06 | 1334 |
| Phage-1019 | — | — | D | — | — | L | A | L | H | M | K | — | — | — | 0.08 | 1.49 | 0.08 | 1335 |
| Phage-1020 | — | — | E | — | — | I | M | — | — | M | A | Q | — | — | 0.06 | 1.49 | 0.06 | 1336 |
| Phage-1021 | — | — | D | — | — | L | P | I | — | Q | A | — | — | — | 0.06 | 1.49 | 0.10 | 1337 |
| Phage-1022 | — | — | D | — | — | S | E | — | — | — | A | — | — | — | 0.06 | 1.49 | 0.08 | 1338 |
| Phage-1023 | — | — | D | — | — | L | P | E | — | — | T | — | — | — | 0.06 | 1.49 | 0.06 | 1339 |
| Phage-1024 | — | — | E | — | — | R | P | E | — | M | Q | — | — | V | 0.06 | 1.48 | 0.09 | 1340 |
| Phage-1025 | — | — | — | — | — | T | V | E | N | M | A | — | — | — | 0.07 | 1.48 | 0.07 | 1341 |
| Phage-1026 | — | — | E | — | — | V | L | E | — | R | G | — | — | D | 0.07 | 1.48 | 0.09 | 1342 |
| Phage-1027 | — | — | D | — | — | V | A | E | — | R | H | — | — | — | 0.05 | 1.47 | 0.06 | 1343 |
| Phage-1028 | — | — | E | — | — | L | D | — | — | M | T | — | — | — | 0.06 | 1.47 | 0.08 | 1344 |
| Phage-1029 | — | — | — | — | — | T | Q | E | — | — | E | — | — | — | 0.06 | 1.47 | 0.06 | 1345 |
| Phage-1030 | — | — | D | — | — | L | A | I | — | — | G | — | — | — | 0.07 | 1.47 | 0.08 | 1346 |
| Phage-1031 | — | — | D | — | — | L | E | I | — | M | S | — | — | — | 0.08 | 1.46 | 0.08 | 1347 |
| Phage-1032 | — | — | D | — | — | L | L | E | — | M | S | — | — | — | 0.06 | 1.46 | 0.09 | 1348 |
| Phage-1033 | — | — | D | — | — | S | L | E | — | M | V | — | — | — | 0.06 | 1.46 | 0.06 | 1349 |
| Phage-1034 | — | — | E | — | — | S | L | E | H | — | A | — | — | — | 0.05 | 1.46 | 0.07 | 1350 |
| Phage-1035 | — | — | E | — | — | T | P | V | — | M | P | — | — | — | 0.08 | 1.46 | 0.06 | 1351 |
| Phage-1036 | — | — | Q | — | — | I | E | P | — | L | A | — | — | — | 0.06 | 1.46 | 0.05 | 1352 |
| Phage-1037 | — | — | D | — | — | L | A | I | — | M | W | — | — | — | 0.07 | 1.45 | 0.09 | 1353 |
| Phage-1038 | — | — | D | — | — | — | E | E | — | M | A | — | — | — | 0.06 | 1.45 | 0.06 | 1354 |
| Phage-1039 | — | — | D | — | — | S | L | — | — | M | S | — | — | — | 0.06 | 1.45 | 0.06 | 1355 |
| Phage-1040 | — | — | E | — | — | L | P | — | — | M | T | — | — | I | 0.07 | 1.44 | 0.08 | 1356 |
| Phage-1041 | — | — | — | — | — | L | P | — | — | — | Q | — | — | — | 0.06 | 1.44 | 0.07 | 1357 |
| Phage-1042 | — | — | E | — | — | L | A | I | — | Q | K | — | — | — | 0.06 | 1.44 | 0.06 | 1358 |
| Phage-1043 | — | — | E | — | — | — | V | E | — | M | A | — | — | — | 0.05 | 1.44 | 0.07 | 1359 |
| Phage-1044 | — | — | E | — | — | L | P | I | — | M | E | — | — | — | 0.10 | 1.44 | 0.07 | 1360 |
| Phage-1045 | — | — | E | — | — | S | M | E | — | — | Q | — | — | — | 0.08 | 1.44 | 0.07 | 1361 |
| Phage-1046 | — | — | E | — | — | S | Q | E | — | M | A | — | — | — | 0.05 | 1.44 | 0.06 | 1362 |
| Phage-1047 | — | — | D | — | — | L | P | E | — | M | — | Q | — | — | 0.05 | 1.44 | 0.07 | 1363 |
| Phage-1048 | — | — | D | — | — | N | F | — | — | M | E | — | — | — | 0.07 | 1.44 | 0.06 | 1364 |
| Phage-1049 | — | — | V | — | — | L | P | — | — | M | Q | — | — | — | 0.07 | 1.43 | 0.06 | 1365 |
| Phage-1050 | — | — | D | — | — | L | P | E | — | — | A | — | — | Q | 0.06 | 1.43 | 0.07 | 1366 |
| Phage-1051 | — | — | D | — | — | S | L | E | — | — | E | — | — | — | 0.06 | 1.43 | 0.06 | 1367 |
| Phage-1052 | — | — | L | — | — | S | — | P | — | — | H | — | — | Y | 0.05 | 1.43 | 0.07 | 1368 |
| Phage-1053 | — | — | D | — | — | L | D | E | — | M | L | — | — | — | 0.06 | 1.43 | 0.06 | 1369 |
| Phage-1054 | — | — | D | — | — | T | P | E | — | M | W | — | — | — | 0.06 | 1.43 | 0.06 | 1370 |
| Phage-1055 | — | — | E | — | — | S | Q | — | — | M | Q | — | — | — | 0.06 | 1.42 | 0.08 | 1371 |
| Phage-1056 | — | — | D | — | — | S | P | A | F | — | Q | — | — | — | 0.07 | 1.42 | 0.08 | 1372 |
| Phage-1057 | — | — | D | — | — | S | L | E | — | — | D | — | — | M | 0.07 | 1.42 | 0.09 | 1373 |
| Phage-1058 | — | — | Q | — | — | S | P | N | — | — | T | — | — | F | 0.06 | 1.41 | 0.06 | 1374 |
| Phage-1059 | — | — | D | — | — | — | P | E | — | L | G | — | — | — | 0.06 | 1.41 | 0.06 | 1375 |
| Phage-1060 | — | — | — | — | — | — | E | E | — | L | E | — | — | — | 0.06 | 1.41 | 0.15 | 1376 |
| Phage-1061 | — | — | D | — | — | S | H | E | — | M | Q | — | — | — | 0.06 | 1.41 | 0.06 | 1377 |
| Phage-1062 | — | — | — | — | — | S | A | E | — | M | S | — | — | — | 0.06 | 1.41 | 0.07 | 1378 |
| Phage-1063 | — | — | D | — | — | L | M | E | — | K | A | — | — | — | 0.06 | 1.41 | 0.06 | 1379 |
| Phage-1064 | — | — | E | — | — | S | A | E | — | — | A | — | — | — | 0.07 | 1.41 | 0.09 | 1380 |
| Phage-1065 | — | — | D | — | — | S | P | E | H | M | G | — | — | — | 0.06 | 1.40 | 0.09 | 1381 |
| Phage-1066 | — | — | D | — | — | D | A | R | — | M | E | — | — | — | 0.07 | 1.40 | 0.06 | 1382 |
| Phage-1067 | — | — | D | — | — | S | P | E | — | M | Q | — | — | — | 0.07 | 1.40 | 0.53 | 1383 |
| Phage-1068 | — | — | N | — | — | L | P | E | — | M | S | — | — | I | 0.06 | 1.40 | 0.07 | 1384 |
| Phage-1069 | — | — | S | — | — | L | P | E | — | M | D | — | — | — | 0.05 | 1.39 | 0.07 | 1385 |
| Phage-1070 | — | — | D | — | — | S | L | E | — | M | L | — | — | — | 0.05 | 1.39 | 0.07 | 1386 |
| Phage-1071 | — | — | E | — | — | H | P | W | — | V | Q | — | — | — | 0.07 | 1.39 | 0.07 | 1387 |
| Phage-1072 | — | — | D | — | — | N | — | E | — | M | E | — | — | — | 0.05 | 1.38 | 0.05 | 1388 |
| Phage-1073 | — | — | D | — | — | — | E | — | — | M | S | — | — | — | 0.07 | 1.38 | 0.07 | 1389 |
| Phage-1074 | — | — | D | — | L | F | Q | L | — | M | M | — | — | — | 0.07 | 1.38 | 0.07 | 1390 |
| Phage-1075 | — | — | D | — | — | — | P | E | — | — | Q | — | — | — | 0.07 | 1.38 | 0.06 | 1391 |
| Phage-1076 | — | — | E | — | — | L | D | E | — | L | Q | — | — | — | 0.06 | 1.38 | 0.07 | 1392 |
| Phage-1077 | — | — | D | — | — | L | Q | E | — | M | Q | — | — | — | 0.06 | 1.38 | 0.06 | 1393 |
| Phage-1078 | — | — | D | — | — | Q | P | — | — | M | S | — | — | — | 0.07 | 1.38 | 0.09 | 1394 |
| Phage-1079 | — | — | D | — | — | L | S | E | — | — | G | — | — | — | 0.06 | 1.37 | 0.06 | 1395 |
| Phage-1080 | — | — | E | — | — | S | Q | L | — | M | A | — | — | — | 0.07 | 1.37 | 0.07 | 1396 |
| Phage-1081 | — | — | D | — | — | S | E | T | — | L | W | — | — | — | 0.07 | 1.37 | 0.10 | 1397 |
| Phage-1082 | — | — | E | — | — | L | P | A | — | M | G | — | — | I | 0.07 | 1.37 | 0.09 | 1398 |
| Phage-1083 | — | — | E | — | — | L | P | E | — | M | I | — | — | I | 0.06 | 1.36 | 0.10 | 1399 |
| Phage-1084 | — | — | — | — | — | T | S | E | — | M | S | — | — | — | 0.06 | 1.36 | 0.07 | 1400 |
| Phage-1085 | — | — | D | — | — | M | K | E | — | M | L | — | — | — | 0.05 | 1.36 | 0.06 | 1401 |
| Phage-1086 | — | — | D | — | — | V | K | E | — | M | Y | — | — | — | 0.06 | 1.36 | 0.06 | 1402 |
| Phage-1087 | — | — | D | — | — | L | A | L | — | M | W | — | — | — | 0.11 | 1.36 | 0.17 | 1403 |
| Phage-1088 | — | — | D | — | — | L | S | I | — | M | T | — | — | — | 0.06 | 1.36 | 0.07 | 1404 |
| Phage-1089 | — | — | D | — | — | M | V | — | — | M | A | — | — | — | 0.06 | 1.36 | 0.07 | 1405 |
| Phage-1090 | — | — | D | — | — | S | E | E | F | M | W | — | — | — | 0.07 | 1.35 | 0.06 | 1406 |
| Phage-1091 | — | — | E | — | — | M | E | K | — | M | E | — | — | — | 0.07 | 1.35 | 0.11 | 1407 |
| Phage-1092 | — | — | D | — | — | L | P | R | — | M | A | — | — | — | 0.06 | 1.35 | 0.10 | 1408 |
| Phage-1093 | — | — | D | — | — | L | P | E | — | M | Q | — | — | — | 0.09 | 1.35 | 0.08 | 1409 |
| Phage-1094 | — | — | D | — | — | L | L | — | — | M | H | — | — | — | 0.10 | 1.35 | 0.07 | 1410 |
| Phage-1095 | — | — | D | — | — | — | P | L | — | — | Q | — | — | — | 0.07 | 1.34 | 0.06 | 1411 |
| Phage-1096 | — | — | D | — | — | S | K | E | — | M | A | — | — | — | 0.07 | 1.34 | 0.06 | 1412 |
| Phage-1097 | — | — | E | — | — | S | P | E | — | M | H | — | — | — | 0.07 | 1.34 | 0.09 | 1413 |
| Phage-1098 | — | — | D | — | — | S | E | A | — | — | E | — | — | — | 0.08 | 1.34 | 0.06 | 1414 |
| Phage-1099 | — | — | — | — | — | V | D | — | — | L | W | — | — | — | 0.07 | 1.34 | 0.06 | 1415 |
| Phage-1100 | — | — | — | — | — | — | S | E | — | M | A | — | — | — | 0.07 | 1.33 | 0.08 | 1416 |
| Phage-1101 | — | — | D | — | — | — | P | — | — | M | R | — | — | — | 0.08 | 1.33 | 0.08 | 1417 |
| Phage-1102 | — | — | D | — | — | L | P | L | — | M | Q | — | — | F | 0.06 | 1.33 | 0.10 | 1418 |
| Phage-1103 | — | — | — | — | — | L | P | M | — | M | Q | — | — | — | 0.06 | 1.33 | 0.08 | 1419 |
| Phage-1104 | — | — | S | E | — | Q | I | I | F | — | Q | — | — | — | 0.07 | 1.32 | 0.08 | 1420 |
| Phage-1105 | — | — | E | — | — | L | E | I | — | L | A | — | — | — | 0.06 | 1.32 | 0.07 | 1421 |
| Phage-1106 | — | — | D | — | — | T | A | E | F | — | G | — | — | — | 0.06 | 1.32 | 0.06 | 1422 |
| Phage-1107 | — | — | — | — | — | — | L | E | — | M | G | — | — | — | 0.07 | 1.32 | 0.06 | 1423 |
| Phage-1108 | — | — | E | — | — | L | P | E | — | M | I | — | — | — | 0.06 | 1.32 | 0.06 | 1424 |
| Phage-1109 | — | — | E | — | — | L | P | A | — | M | W | — | — | — | 0.06 | 1.31 | 0.08 | 1425 |
| Phage-1110 | — | — | E | — | — | L | P | M | — | M | Q | — | — | — | 0.07 | 1.31 | 0.08 | 1426 |
| Phage-1111 | — | — | D | — | — | I | P | E | — | M | T | — | — | — | 0.06 | 1.31 | 0.06 | 1427 |
| Phage-1112 | — | — | D | — | — | S | A | E | — | — | A | — | — | — | 0.05 | 1.31 | 0.05 | 1428 |
| Phage-1113 | — | — | E | — | — | S | A | V | — | M | S | — | — | — | 0.06 | 1.31 | 0.06 | 1429 |
| Phage-1114 | — | — | E | — | — | S | L | E | — | M | M | — | — | V | 0.06 | 1.30 | 0.07 | 1430 |
| Phage-1115 | — | — | D | — | — | — | P | — | — | M | A | — | — | I | 0.06 | 1.30 | 0.06 | 1431 |
| Phage-1116 | — | — | Q | — | — | S | V | E | H | M | A | — | — | — | 0.06 | 1.30 | 0.06 | 1432 |
| Phage-1117 | — | — | — | — | — | M | P | I | — | M | T | — | — | — | 0.06 | 1.30 | 0.08 | 1433 |
| Phage-1118 | — | — | D | — | — | R | A | E | — | M | L | — | — | — | 0.06 | 1.29 | 0.08 | 1434 |
| Phage-1119 | — | — | E | — | — | L | V | — | — | M | Q | — | — | — | 0.06 | 1.28 | 0.06 | 1435 |
| Phage-1120 | — | — | M | — | — | L | P | I | — | M | E | — | — | — | 0.05 | 1.28 | 0.05 | 1436 |
| Phage-1121 | — | — | E | — | — | L | P | R | — | M | L | — | — | — | 0.06 | 1.28 | 0.30 | 1437 |
| Phage-1122 | — | — | D | — | — | L | E | V | — | M | D | — | — | — | 0.13 | 1.28 | 0.07 | 1438 |
| Phage-1123 | — | — | D | — | — | S | P | A | — | L | Q | — | — | — | 0.07 | 1.28 | 0.06 | 1439 |
| Phage-1124 | — | — | E | — | — | S | S | — | W | M | W | — | — | — | 0.06 | 1.27 | 0.09 | 1440 |
| Phage-1125 | — | — | E | — | — | F | P | L | — | M | — | — | — | — | 0.06 | 1.27 | 0.08 | 1441 |
| Phage-1126 | — | — | E | — | — | S | T | E | — | M | A | — | — | — | 0.08 | 1.27 | 0.06 | 1442 |
| Phage-1127 | — | — | E | — | — | Q | P | A | — | M | E | — | — | — | 0.05 | 1.27 | 0.06 | 1443 |
| Phage-1128 | — | — | D | — | — | — | E | E | — | R | M | — | — | — | 0.05 | 1.27 | 0.08 | 1444 |
| Phage-1129 | — | — | D | — | — | L | P | V | — | M | T | — | — | — | 0.07 | 1.27 | 0.07 | 1445 |
| Phage-1130 | — | — | D | — | — | S | S | V | — | M | L | — | — | — | 0.08 | 1.26 | 0.06 | 1446 |
| Phage-1131 | — | — | E | — | — | H | R | I | — | M | Q | — | — | — | 0.05 | 1.26 | 0.07 | 1447 |
| Phage-1132 | — | — | E | — | — | S | E | E | — | M | A | — | — | — | 0.06 | 1.26 | 0.07 | 1448 |
| Phage-1133 | — | — | E | — | — | M | S | I | — | M | Q | — | — | — | 0.07 | 1.25 | 0.07 | 1449 |
| Phage-1134 | — | — | E | — | — | L | Q | E | H | M | A | — | — | — | 0.06 | 1.25 | 0.05 | 1450 |
| Phage-1135 | — | — | — | — | L | F | E | E | — | M | S | — | — | — | 0.06 | 1.25 | 0.06 | 1451 |
| Phage-1136 | — | — | E | — | — | L | P | M | — | M | T | — | — | — | 0.05 | 1.25 | 0.06 | 1452 |
| Phage-1137 | — | — | D | — | — | S | E | — | — | M | A | — | — | — | 0.06 | 1.24 | 0.06 | 1453 |
| Phage-1138 | — | — | E | — | — | T | Q | E | — | M | Q | — | — | — | 0.07 | 1.24 | 0.07 | 1454 |
| Phage-1139 | — | — | — | — | — | T | A | E | — | M | A | — | — | — | 0.06 | 1.24 | 0.08 | 1455 |
| Phage-1140 | — | — | E | — | — | L | A | E | — | M | — | — | — | — | 0.06 | 1.24 | 0.07 | 1456 |
| Phage-1141 | — | — | D | — | — | V | E | E | — | M | T | — | — | — | 0.06 | 1.24 | 0.07 | 1457 |
| Phage-1142 | — | — | D | — | — | L | E | — | — | M | L | — | — | — | 0.07 | 1.24 | 0.09 | 1458 |
| Phage-1143 | — | — | E | — | — | I | G | I | — | M | S | — | — | — | 0.07 | 1.23 | 0.07 | 1459 |
| Phage-1144 | — | — | E | — | — | S | T | E | — | — | Q | — | — | — | 0.06 | 1.23 | 0.09 | 1460 |
| Phage-1145 | — | — | D | — | — | S | M | M | — | M | Q | — | — | — | 0.06 | 1.23 | 0.06 | 1461 |
| Phage-1146 | — | — | E | — | — | S | A | E | H | M | P | — | — | — | 0.07 | 1.23 | 0.09 | 1462 |
| Phage-1147 | — | — | V | — | — | Y | P | E | — | M | E | — | — | — | 0.06 | 1.22 | 0.07 | 1463 |
| Phage-1148 | — | — | D | — | — | E | A | R | — | M | Q | — | — | — | 0.09 | 1.21 | 0.07 | 1464 |
| Phage-1149 | — | — | D | — | — | S | Q | E | — | M | S | — | — | — | 0.06 | 1.21 | 0.06 | 1465 |
| Phage-1150 | — | — | H | — | — | L | P | — | — | M | S | — | — | M | 0.07 | 1.21 | 0.06 | 1466 |
| Phage-1151 | — | — | — | — | — | L | D | I | — | L | A | — | — | — | 0.06 | 1.21 | 0.07 | 1467 |
| Phage-1152 | — | — | — | — | — | — | P | E | — | — | S | — | — | — | 0.08 | 1.21 | 0.07 | 1468 |
| Phage-1153 | — | — | D | — | — | S | — | E | — | L | G | — | — | — | 0.05 | 1.20 | 0.05 | 1469 |
| Phage-1154 | — | — | E | — | — | — | A | E | — | L | W | — | — | — | 0.05 | 1.18 | 0.09 | 1470 |
| Phage-1155 | — | — | D | — | — | R | P | E | — | M | S | — | — | — | 0.05 | 1.18 | 0.07 | 1471 |
| Phage-1156 | — | — | E | — | — | M | P | E | — | L | A | — | — | — | 0.06 | 1.18 | 0.07 | 1472 |
| Phage-1157 | — | — | E | — | — | — | G | V | — | M | A | — | — | — | 0.06 | 1.17 | 0.05 | 1473 |
| Phage-1158 | — | — | E | — | — | S | Q | T | — | M | W | — | — | — | 0.07 | 1.17 | 0.08 | 1474 |
| Phage-1159 | — | — | — | — | — | S | S | E | — | — | G | — | — | — | 0.05 | 1.17 | 0.06 | 1475 |
| Phage-1160 | — | — | E | — | — | S | Q | — | — | M | W | — | — | — | 0.05 | 1.17 | 0.05 | 1476 |
| Phage-1161 | — | — | D | — | — | L | P | D | — | M | A | — | — | — | 0.06 | 1.17 | 0.07 | 1477 |
| Phage-1162 | — | — | E | — | — | — | E | — | — | M | E | — | — | — | 0.07 | 1.17 | 0.08 | 1478 |
| Phage-1163 | — | — | E | — | — | L | K | E | — | M | Q | — | — | — | 0.06 | 1.17 | 0.07 | 1479 |
| Phage-1164 | — | — | E | — | — | S | Y | — | — | M | Q | — | — | — | 0.06 | 1.16 | 0.06 | 1480 |
| Phage-1165 | — | — | E | — | — | R | A | E | — | M | S | — | — | — | 0.07 | 1.16 | 0.09 | 1481 |
| Phage-1166 | — | — | D | — | — | S | — | I | — | M | V | — | — | — | 0.06 | 1.16 | 0.06 | 1482 |
| Phage-1167 | — | — | D | — | — | L | S | V | — | M | S | — | — | — | 0.06 | 1.15 | 0.07 | 1483 |
| Phage-1168 | — | — | E | — | — | L | P | — | — | M | S | — | — | — | 0.06 | 1.15 | 0.09 | 1484 |
| Phage-1169 | — | — | E | — | — | V | A | E | — | R | A | — | — | — | 0.06 | 1.15 | 0.09 | 1485 |
| Phage-1170 | — | — | Q | — | — | L | P | — | — | — | E | — | — | — | 0.06 | 1.14 | 0.06 | 1486 |
| Phage-1171 | — | — | E | — | — | — | A | A | — | M | G | — | — | — | 0.06 | 1.14 | 0.08 | 1487 |
| Phage-1172 | — | — | D | — | — | E | Q | — | — | M | E | — | — | — | 0.06 | 1.14 | 0.06 | 1488 |
| Phage-1173 | — | — | D | — | — | L | Q | M | — | M | M | — | — | — | 0.08 | 1.14 | 0.07 | 1489 |
| Phage-1174 | — | — | E | — | — | L | Y | E | — | M | W | — | — | — | 0.06 | 1.13 | 0.07 | 1490 |
| Phage-1175 | — | — | V | — | — | S | E | — | — | M | A | — | — | — | 0.07 | 1.13 | 0.06 | 1491 |
| Phage-1176 | — | — | D | — | — | E | A | E | — | M | W | — | — | — | 0.09 | 1.13 | 0.10 | 1492 |
| Phage-1177 | — | — | E | — | — | — | E | — | — | M | K | — | — | — | 0.06 | 1.12 | 0.07 | 1493 |
| Phage-1178 | — | — | D | — | — | V | V | E | — | M | S | — | — | — | 0.09 | 1.12 | 0.08 | 1494 |
| Phage-1179 | — | — | — | — | — | L | P | — | — | M | H | — | — | — | 0.06 | 1.11 | 0.08 | 1495 |
| Phage-1180 | — | — | D | — | — | I | — | P | W | — | E | — | — | — | 0.06 | 1.11 | 0.11 | 1496 |
| Phage-1181 | — | — | E | — | — | S | S | L | — | L | E | — | — | — | 0.07 | 1.11 | 0.08 | 1497 |
| Phage-1182 | — | — | D | — | — | — | M | E | — | M | E | — | — | — | 0.07 | 1.11 | 0.08 | 1498 |
| Phage-1183 | — | — | E | — | — | L | — | E | — | L | A | — | — | — | 0.06 | 1.10 | 0.05 | 1499 |
| Phage-1184 | — | — | E | — | — | M | G | E | — | M | L | — | — | — | 0.07 | 1.10 | 0.06 | 1500 |
| Phage-1185 | — | — | D | — | — | T | K | E | — | M | W | — | — | — | 0.06 | 1.10 | 0.06 | 1501 |
| Phage-1186 | — | — | — | — | — | — | E | E | — | M | L | — | — | — | 0.05 | 1.09 | 0.07 | 1502 |
| Phage-1187 | — | — | E | — | — | S | A | E | — | — | G | — | — | — | 0.07 | 1.08 | 0.07 | 1503 |
| Phage-1188 | — | — | V | — | — | L | P | E | — | M | L | E | — | M | 0.06 | 1.08 | 0.07 | 1504 |
| Phage-1189 | — | — | D | — | — | S | F | H | — | M | A | — | — | — | 0.06 | 1.08 | 0.06 | 1505 |
| Phage-1190 | — | — | E | — | — | S | E | M | — | M | S | — | — | — | 0.06 | 1.07 | 0.09 | 1506 |
| Phage-1191 | — | — | D | — | — | — | N | A | — | — | W | — | — | — | 0.06 | 1.07 | 0.09 | 1507 |
| Phage-1192 | — | — | E | — | — | I | P | M | — | M | L | — | — | M | 0.06 | 1.06 | 0.09 | 1508 |
| Phage-1193 | — | — | E | — | — | — | Q | E | — | M | A | — | — | — | 0.07 | 1.06 | 0.08 | 1509 |
| Phage-1194 | — | — | D | — | — | — | K | V | — | — | W | — | — | — | 0.06 | 1.06 | 0.08 | 1510 |
| Phage-1195 | — | — | E | — | — | I | P | A | — | M | A | — | — | — | 0.06 | 1.06 | 0.08 | 1511 |
| Phage-1196 | — | — | D | — | — | — | P | I | — | M | Q | — | — | — | 0.06 | 1.06 | 0.06 | 1512 |
| Phage-1197 | — | — | E | — | — | L | — | E | — | M | K | — | — | — | 0.07 | 1.06 | 0.07 | 1513 |
| Phage-1198 | — | — | E | — | — | D | V | — | — | M | Q | — | — | — | 0.07 | 1.06 | 0.08 | 1514 |
| Phage-1199 | — | — | E | — | — | S | S | E | — | L | G | — | — | — | 0.05 | 1.05 | 0.09 | 1515 |
| Phage-1200 | — | — | D | — | — | L | P | R | — | M | E | — | — | M | 0.06 | 1.05 | 0.06 | 1516 |
| Phage-1201 | — | — | D | — | — | S | S | A | — | — | D | — | — | — | 0.09 | 1.05 | 0.06 | 1517 |
| Phage-1202 | — | — | E | — | — | L | P | S | — | L | A | — | — | — | 0.06 | 1.05 | 0.06 | 1518 |
| Phage-1203 | — | — | D | — | — | — | P | E | F | — | R | — | — | — | 0.07 | 1.05 | 0.07 | 1519 |
| Phage-1204 | — | — | E | — | — | — | P | — | — | M | S | — | — | — | 0.06 | 1.04 | 0.08 | 1520 |
| Phage-1205 | — | — | D | — | — | D | A | R | — | M | T | — | — | — | 0.06 | 1.04 | 0.09 | 1521 |
| Phage-1206 | — | — | E | — | — | S | V | E | — | — | L | — | — | — | 0.11 | 1.04 | 0.06 | 1522 |
| Phage-1207 | — | — | E | — | — | S | S | A | — | M | R | — | — | — | 0.06 | 1.04 | 0.06 | 1523 |
| Phage-1208 | — | — | D | — | — | L | A | E | — | — | Q | — | — | — | 0.06 | 1.04 | 0.08 | 1524 |
| Phage-1209 | — | — | — | — | — | S | S | E | — | L | Q | — | — | — | 0.07 | 1.03 | 0.09 | 1525 |
| Phage-1210 | — | — | — | — | — | S | Q | V | — | M | W | — | — | — | 0.05 | 1.02 | 0.06 | 1526 |
| Phage-1211 | — | — | E | — | — | I | E | — | — | M | R | — | — | — | 0.06 | 1.01 | 0.06 | 1527 |
| Phage-1212 | — | — | F | — | — | — | P | E | — | L | L | — | — | — | 0.08 | 1.01 | 0.08 | 1528 |
| Phage-1213 | — | — | E | — | — | L | R | E | — | M | L | — | — | — | 0.06 | 1.01 | 0.07 | 1529 |
| Phage-1214 | — | — | E | — | — | S | S | V | — | L | W | — | — | — | 0.05 | 1.00 | 0.05 | 1530 |
| Phage-1215 | — | — | E | — | — | S | — | I | — | — | A | — | — | — | 0.05 | 1.00 | 0.07 | 1531 |
| Phage-1216 | — | — | D | — | — | L | E | V | — | M | S | — | — | — | 0.06 | 1.00 | 0.09 | 1532 |
| Phage-1217 | — | — | E | — | — | R | P | E | — | M | A | N | — | — | 0.05 | 1.00 | 0.06 | 1533 |
| Phage-1218 | — | — | E | — | — | I | E | P | — | M | Q | — | — | — | 0.05 | 0.99 | 0.06 | 1534 |
| Phage-1219 | — | — | D | — | — | K | P | E | — | M | D | — | — | — | 0.06 | 0.99 | 0.07 | 1535 |
| Phage-1220 | — | — | D | — | — | V | G | A | — | M | W | — | — | — | 0.05 | 0.99 | 0.06 | 1536 |
| Phage-1221 | — | — | E | — | — | S | A | I | — | — | A | — | — | M | 0.09 | 0.98 | 0.11 | 1537 |
| Phage-1222 | — | — | F | — | — | F | P | E | — | M | A | — | — | — | 0.07 | 0.98 | 0.07 | 1538 |
| Phage-1223 | — | — | D | — | — | T | G | M | — | — | S | — | — | — | 0.06 | 0.98 | 0.06 | 1539 |
| Phage-1224 | — | — | S | — | — | S | — | A | — | — | G | — | — | — | 0.08 | 0.98 | 0.07 | 1540 |
| Phage-1225 | — | — | — | — | — | — | A | E | — | M | W | — | — | — | 0.05 | 0.97 | 0.06 | 1541 |
| Phage-1226 | — | — | Q | — | — | F | P | — | — | M | A | — | — | — | 0.05 | 0.97 | 0.07 | 1542 |
| Phage-1227 | — | — | E | — | — | V | P | E | — | L | V | — | — | — | 0.07 | 0.96 | 0.06 | 1543 |
| Phage-1228 | — | — | D | — | — | S | K | E | — | M | S | — | — | — | 0.06 | 0.96 | 0.07 | 1544 |
| Phage-1229 | — | — | D | — | — | L | P | E | — | M | L | — | — | — | 0.06 | 0.96 | 0.09 | 1545 |
| Phage-1230 | — | — | — | — | — | L | D | V | — | M | S | — | — | — | 0.08 | 0.96 | 0.09 | 1546 |
| Phage-1231 | — | — | — | — | — | T | P | E | — | L | D | — | — | — | 0.07 | 0.95 | 0.06 | 1547 |
| Phage-1232 | — | — | E | — | — | D | I | R | — | M | M | — | — | — | 0.08 | 0.95 | 0.08 | 1548 |
| Phage-1233 | — | — | D | — | — | E | E | R | — | M | A | — | — | — | 0.06 | 0.94 | 0.06 | 1549 |
| Phage-1234 | — | — | S | — | — | V | P | E | — | M | E | — | — | — | 0.07 | 0.94 | 0.06 | 1550 |
| Phage-1235 | — | — | D | — | — | I | P | — | — | V | E | — | — | — | 0.06 | 0.94 | 0.08 | 1551 |
| Phage-1236 | — | — | — | — | — | — | E | I | — | M | Q | — | — | — | 0.05 | 0.93 | 0.05 | 1552 |
| Phage-1237 | — | — | E | — | — | — | H | E | — | M | E | — | — | V | 0.06 | 0.93 | 0.07 | 1553 |
| Phage-1238 | — | — | D | — | — | H | A | E | — | M | W | — | — | — | 0.05 | 0.93 | 0.06 | 1554 |
| Phage-1239 | — | — | E | — | — | T | Q | A | — | L | A | — | — | — | 0.07 | 0.93 | 0.10 | 1555 |
| Phage-1240 | — | — | — | — | — | S | A | — | — | M | W | — | — | — | 0.05 | 0.92 | 0.07 | 1556 |
| Phage-1241 | — | — | D | — | — | S | F | E | — | M | A | — | — | — | 0.06 | 0.92 | 0.06 | 1557 |
| Phage-1242 | — | — | D | — | — | S | A | E | — | L | G | — | — | — | 0.07 | 0.92 | 0.07 | 1558 |
| Phage-1243 | — | — | V | — | — | S | P | E | — | — | W | — | — | — | 0.07 | 0.92 | 0.06 | 1559 |
| Phage-1244 | — | — | D | — | — | E | L | M | — | M | E | — | — | — | 0.05 | 0.92 | 0.06 | 1560 |
| Phage-1245 | — | — | — | — | — | L | E | M | — | M | S | — | — | — | 0.06 | 0.91 | 0.07 | 1561 |
| Phage-1246 | — | — | D | — | — | I | K | E | — | M | S | — | — | — | 0.05 | 0.91 | 0.08 | 1562 |
| Phage-1247 | — | — | D | — | — | L | L | M | — | M | E | — | — | — | 0.05 | 0.91 | 0.06 | 1563 |
| Phage-1248 | — | — | E | — | — | D | — | M | — | M | E | — | — | — | 0.07 | 0.91 | 0.07 | 1564 |
| Phage-1249 | — | — | E | — | — | S | Q | E | — | K | A | — | — | F | 0.07 | 0.90 | 0.07 | 1565 |
| Phage-1250 | — | — | D | — | — | V | L | M | — | L | A | — | — | — | 0.07 | 0.90 | 0.09 | 1566 |
| Phage-1251 | — | — | E | — | — | S | S | A | — | M | A | — | — | — | 0.15 | 0.90 | 0.07 | 1567 |
| Phage-1252 | — | — | E | — | — | L | A | A | — | M | Y | — | — | — | 0.05 | 0.89 | 0.07 | 1568 |
| Phage-1253 | — | — | D | — | — | S | T | L | — | M | Q | — | — | — | 0.07 | 0.88 | 0.06 | 1569 |
| Phage-1254 | — | — | D | — | — | T | E | A | — | M | S | — | — | — | 0.07 | 0.88 | 0.06 | 1570 |
| Phage-1255 | — | — | E | — | — | I | S | E | — | M | G | — | — | — | 0.07 | 0.88 | 0.05 | 1571 |
| Phage-1256 | — | — | D | — | — | F | T | W | — | M | A | — | — | — | 0.07 | 0.87 | 0.08 | 1572 |
| Phage-1257 | — | — | D | — | — | I | A | E | — | R | S | — | — | — | 0.11 | 0.87 | 0.07 | 1573 |
| Phage-1258 | — | — | E | — | — | L | S | E | — | — | G | — | — | — | 0.06 | 0.87 | 0.06 | 1574 |
| Phage-1259 | — | — | D | — | — | T | A | — | — | M | W | — | — | — | 0.07 | 0.87 | 0.07 | 1575 |
| Phage-1260 | — | — | — | — | — | S | L | — | — | L | G | — | — | Y | 0.06 | 0.86 | 0.06 | 1576 |
| Phage-1261 | — | — | — | — | — | — | A | I | — | M | A | — | — | — | 0.06 | 0.85 | 0.07 | 1577 |
| Phage-1262 | — | — | D | — | — | S | M | T | — | M | A | — | — | I | 0.06 | 0.84 | 0.06 | 1578 |
| Phage-1263 | — | — | D | — | — | K | — | E | — | L | Q | — | — | — | 0.06 | 0.84 | 0.06 | 1579 |
| Phage-1264 | — | — | D | — | — | D | P | — | — | — | R | — | — | I | 0.08 | 0.83 | 0.06 | 1580 |
| Phage-1265 | — | — | E | — | — | L | P | M | — | M | Q | — | — | M | 0.05 | 0.82 | 0.07 | 1581 |
| Phage-1266 | — | — | D | — | — | S | G | E | — | M | S | — | — | — | 0.06 | 0.82 | 0.06 | 1582 |
| Phage-1267 | — | — | E | — | — | — | R | E | — | M | D | — | — | — | 0.07 | 0.80 | 0.07 | 1583 |
| Phage-1268 | — | — | D | — | — | S | M | E | — | M | A | — | — | — | 0.05 | 0.80 | 0.07 | 1584 |
| Phage-1269 | — | — | D | — | — | S | P | — | — | L | W | — | — | — | 0.06 | 0.80 | 0.05 | 1585 |
| Phage-1270 | — | — | — | — | — | S | A | E | — | — | Q | — | — | — | 0.08 | 0.80 | 0.08 | 1586 |
| Phage-1271 | — | — | — | — | — | T | Q | E | — | M | L | — | — | — | 0.07 | 0.78 | 0.06 | 1587 |
| Phage-1272 | — | — | Q | — | — | S | V | — | — | M | A | — | — | — | 0.06 | 0.77 | 0.06 | 1588 |
| Phage-1273 | — | — | E | — | — | S | S | E | — | M | V | — | — | — | 0.06 | 0.77 | 0.06 | 1589 |
| Phage-1274 | — | — | D | — | — | T | G | V | — | K | W | — | — | I | 0.07 | 0.76 | 0.06 | 1590 |
| Phage-1275 | — | — | — | — | — | I | G | E | — | M | D | — | — | — | 0.06 | 0.75 | 0.58 | 1591 |
| Phage-1276 | — | — | D | — | — | — | G | I | — | V | L | — | — | — | 0.06 | 0.75 | 0.08 | 1592 |
| Phage-1277 | — | — | D | — | — | N | P | V | — | L | A | — | — | — | 0.05 | 0.74 | 0.08 | 1593 |
| Phage-1278 | — | — | E | — | — | — | Q | — | — | M | L | — | — | — | 0.06 | 0.74 | 0.05 | 1594 |
| Phage-1279 | — | — | — | — | — | S | V | — | — | M | Y | — | — | — | 0.05 | 0.74 | 0.06 | 1595 |
| Phage-1280 | — | — | E | — | — | F | G | M | — | M | S | — | — | — | 0.06 | 0.73 | 0.07 | 1596 |
| Phage-1281 | — | — | — | — | — | M | P | — | — | M | S | — | — | — | 0.07 | 0.72 | 0.05 | 1597 |
| Phage-1282 | — | — | Q | — | — | — | L | — | — | L | Q | — | — | — | 0.07 | 0.71 | 0.08 | 1598 |
| Phage-1283 | — | — | — | — | — | L | P | — | — | V | S | — | — | — | 0.06 | 0.70 | 0.06 | 1599 |
| Phage-1284 | — | — | E | — | — | L | P | W | — | M | S | — | — | — | 0.06 | 0.69 | 0.09 | 1600 |
| Phage-1285 | — | — | D | — | — | H | L | E | — | — | D | — | — | — | 0.06 | 0.69 | 0.06 | 1601 |
| Phage-1286 | — | — | D | — | — | L | P | R | F | M | V | — | — | — | 0.05 | 0.69 | 0.07 | 1602 |
| Phage-1287 | — | — | Q | — | — | — | P | W | — | M | V | — | — | — | 0.07 | 0.69 | 0.08 | 1603 |
| Phage-1288 | — | — | E | — | — | M | S | E | — | M | R | — | — | — | 0.07 | 0.69 | 0.10 | 1604 |
| Phage-1289 | — | — | V | — | — | L | P | L | — | M | P | N | — | — | 0.09 | 0.68 | 0.11 | 1605 |
| Phage-1290 | — | — | D | — | — | E | P | — | — | M | T | — | — | — | 0.07 | 0.68 | 0.10 | 1606 |
| Phage-1291 | — | — | E | — | — | L | P | — | — | L | A | — | — | Y | 0.07 | 0.67 | 0.08 | 1607 |
| Phage-1292 | — | — | D | — | — | Y | G | A | — | — | L | — | — | — | 0.05 | 0.67 | 0.06 | 1608 |
| Phage-1293 | — | — | E | — | — | — | A | V | — | M | H | — | — | — | 0.14 | 0.67 | 0.09 | 1609 |
| Phage-1294 | — | — | D | — | — | M | D | A | — | M | S | — | — | — | 0.05 | 0.65 | 0.06 | 1610 |
| Phage-1295 | — | — | M | — | — | S | A | E | — | M | G | — | — | — | 0.06 | 0.64 | 0.06 | 1611 |
| Phage-1296 | — | — | D | — | — | — | — | W | — | M | E | — | — | — | 0.05 | 0.64 | 0.06 | 1612 |
| Phage-1297 | — | — | D | — | — | — | A | W | — | L | Q | — | — | — | 0.06 | 0.64 | 0.07 | 1613 |
| Phage-1298 | — | — | E | — | — | — | Q | M | — | M | L | — | — | — | 0.07 | 0.64 | 0.06 | 1614 |
| Phage-1299 | — | — | E | — | — | S | R | — | — | M | E | — | — | M | 0.05 | 0.63 | 0.33 | 1615 |
| Phage-1300 | — | — | S | — | — | L | P | M | — | M | A | — | — | — | 0.06 | 0.62 | 0.13 | 1616 |
| Phage-1301 | — | — | E | — | — | L | A | V | H | M | S | — | — | — | 0.06 | 0.62 | 0.06 | 1617 |
| Phage-1302 | — | — | D | — | — | I | E | M | — | M | S | — | — | M | 0.06 | 0.61 | 0.06 | 1618 |
| Phage-1303 | — | — | — | — | — | — | L | E | — | M | Q | — | — | M | 0.07 | 0.61 | 0.08 | 1619 |
| Phage-1304 | — | — | E | — | — | I | G | D | — | — | A | — | — | — | 0.05 | 0.61 | 0.36 | 1620 |
| Phage-1305 | — | — | Q | — | — | S | E | L | — | M | S | — | — | — | 0.05 | 0.60 | 0.08 | 1621 |
| Phage-1306 | — | — | D | — | — | T | P | — | F | R | A | — | — | — | 0.06 | 0.59 | 0.07 | 1622 |
| Phage-1307 | — | — | E | — | — | L | Q | E | — | M | G | — | — | — | 0.05 | 0.58 | 0.25 | 1623 |
| Phage-1308 | — | — | D | — | — | V | T | E | — | M | A | — | — | — | 0.06 | 0.58 | 0.07 | 1624 |
| Phage-1309 | — | — | D | — | — | I | M | P | — | L | D | — | — | — | 0.06 | 0.57 | 0.05 | 1625 |
| Phage-1310 | — | — | E | — | — | K | K | E | — | M | L | — | — | — | 0.07 | 0.56 | 0.08 | 1626 |
| Phage-1311 | — | — | E | — | — | V | E | L | — | R | Q | — | — | — | 0.06 | 0.56 | 0.06 | 1627 |
| Phage-1312 | — | — | D | — | — | L | E | T | — | M | Q | — | — | — | 0.05 | 0.53 | 0.08 | 1628 |
| Phage-1313 | — | — | Q | — | — | L | P | I | — | — | Q | — | — | — | 0.06 | 0.51 | 0.07 | 1629 |
| Phage-1314 | — | — | E | — | — | L | D | E | — | L | Q | — | — | M | 0.07 | 0.51 | 0.08 | 1630 |
| Phage-1315 | — | — | — | — | — | T | T | E | — | — | V | — | — | — | 0.06 | 0.50 | 0.05 | 1631 |
| Phage-1316 | — | — | E | — | — | V | P | — | — | M | G | — | — | — | 0.08 | 0.50 | 0.08 | 1632 |
| Phage-1317 | — | — | D | — | — | — | A | M | — | M | S | — | — | — | 0.07 | 0.50 | 0.07 | 1633 |
| Phage-1318 | — | — | E | — | — | L | G | L | — | M | K | — | — | — | 0.06 | 0.49 | 0.06 | 1634 |
| Phage-1319 | — | — | — | — | — | — | Q | E | — | M | R | — | — | — | 0.07 | 0.49 | 0.07 | 1635 |
| Phage-1320 | — | — | — | — | — | I | P | — | — | L | — | — | — | — | 0.07 | 0.49 | 0.07 | 1636 |
| Phage-1321 | — | — | D | — | — | S | T | E | — | M | W | — | — | — | 0.05 | 0.49 | 0.06 | 1637 |
| Phage-1322 | — | — | E | — | — | I | K | D | — | M | T | — | — | — | 0.06 | 0.47 | 0.08 | 1638 |
| Phage-1323 | — | — | — | — | — | D | A | E | — | M | Q | — | — | — | 0.07 | 0.46 | 0.06 | 1639 |
| Phage-1324 | — | — | E | — | — | M | Q | W | — | M | A | — | — | — | 0.08 | 0.45 | 0.09 | 1640 |
| Phage-1325 | — | — | E | — | — | — | P | V | — | M | Q | — | — | — | 0.07 | 0.44 | 0.08 | 1641 |
| Phage-1326 | — | — | D | — | — | D | E | — | — | L | M | — | — | — | 0.09 | 0.44 | 0.07 | 1642 |
| Phage-1327 | — | — | D | — | — | L | P | E | — | M | E | — | — | — | 0.08 | 0.43 | 0.07 | 1643 |
| Phage-1328 | — | — | — | — | — | — | S | E | — | L | Q | — | — | — | 0.05 | 0.43 | 0.06 | 1644 |
| Phage-1329 | — | — | D | — | — | S | Q | I | — | M | S | — | — | — | 0.05 | 0.42 | 0.06 | 1645 |
| Phage-1330 | — | — | D | — | — | W | P | G | — | L | W | — | — | — | 0.07 | 0.42 | 0.06 | 1646 |
| Phage-1331 | — | — | T | — | W | Q | V | A | — | M | Q | N | — | F | 0.06 | 0.42 | 0.06 | 1647 |
| Phage-1332 | — | — | — | — | — | L | D | E | — | M | E | — | — | — | 0.07 | 0.40 | 0.07 | 1648 |
| Phage-1333 | — | — | D | — | — | T | — | M | — | K | Q | — | — | — | 0.06 | 0.40 | 0.08 | 1649 |

TABLE 8
Phage Panning Results of Peptide-92 Library Sequences. (—) indicates
the same amino acid position as in Peptide-92 corresponding position.

| | | | | | | | | | | | | | | | Phage binding ELISA | | | |
|---|---|---|---|---|---|---|---|---|---|---|---|---|---|---|---|---|---|---|
| | | | | | | | | | | | | | | | | | Anti- | |
| | | | | | | | | | | | | | | | | Anti- | CD28 | |
| | | | | | | | | | | | | | | | | CD28 | Fab | |
| | | | | | | | | | | | | | | | | Fab | signal | |
| | | | | | | | | | | | | | | | | (SEQ ID | in the | |
| | | | | | | | | | | | | | | | | NOs: | presence | SEQ |
| | Amino acid position sequence | | | | | | | | | | | | | | Background | 696, 697) | of | ID |
| Phage ID | 1 | 2 | 3 | 4 | 5 | 6 | 7 | 8 | 9 | 10 | 11 | 12 | 13 | 14 | signal | signal | CD28 | NO: |
| Phage-471 | S | C | A | A | G | A | S | Y | A | W | P | Y | C | L | 0.05 | 1.49 | 0.05 | 749 |
| Phage-454 | Y | W | S | C | F | Q | I | P | F | T | C | F | Y | F | 0.18 | 1.44 | 0.27 | 732 |
| Phage-455 | W | E | C | L | K | Y | T | E | — | N | A | C | T | Y | 0.05 | 1.55 | 0.07 | 733 |
| Phage-456 | F | M | C | L | P | Q | N | P | H | P | L | C | F | K | 0.06 | 0.84 | 0.06 | 734 |
| Phage-457 | P | D | C | Q | T | I | L | F | K | P | W | C | V | R | 0.05 | 2.09 | 0.06 | 735 |
| Phage-458 | A | — | D | T | Y | F | A | G | M | I | N | E | — | F | 0.07 | 0.92 | 0.10 | 736 |
| Phage-459 | G | — | Q | Y | P | W | A | F | D | A | M | S | — | P | 0.05 | 2.38 | 0.07 | 737 |
| Phage-460 | W | — | W | D | Y | K | L | W | V | P | T | Q | — | P | 0.11 | 2.27 | 0.14 | 738 |
| Phage-461 | W | — | W | D | Y | R | E | W | V | P | M | T | — | P | 0.06 | 2.30 | 0.08 | 739 |
| Phage-462 | E | — | H | W | S | M | W | A | V | G | H | A | — | F | 0.06 | 1.77 | 0.07 | 740 |
| Phage-463 | Y | — | — | G | H | S | P | I | N | I | G | K | — | — | 0.05 | 1.49 | 0.06 | 741 |
| Phage-464 | W | — | W | D | Y | K | L | W | V | P | M | T | — | P | 0.11 | 2.51 | 0.11 | 742 |
| Phage-465 | D | — | W | W | M | F | E | S | Y | P | G | R | — | I | 0.06 | 1.65 | 0.07 | 743 |
| Phage-466 | Y | — | — | G | H | — | W | Q | Y | I | N | K | — | — | 0.06 | 0.64 | 0.07 | 744 |
| Phage-467 | W | — | W | Y | Y | R | E | W | V | P | M | T | — | P | 0.06 | 1.85 | 0.07 | 745 |
| Phage-468 | Y | W | Y | C | S | P | S | I | V | R | C | V | L | V | 0.06 | 1.51 | 0.09 | 746 |
| Phage-469 | P | D | C | H | T | I | L | F | K | P | W | C | V | R | 0.07 | 1.00 | 0.06 | 747 |
| Phage-470 | W | I | C | D | K | S | G | S | I | M | L | C | R | A | 0.06 | 0.91 | 0.06 | 748 |
| Phage-472 | D | — | P | W | L | S | G | F | S | R | — | L | L | C | 0.09 | 1.27 | 0.07 | 750 |
| Phage-473 | Y | — | — | G | M | S | T | I | Q | L | W | L | K | C | 0.06 | 2.16 | 0.09 | 751 |
| Phage-474 | Y | — | — | G | H | M | P | Q | Y | E | V | K | — | — | 0.07 | 0.22 | 0.06 | 752 |
| Phage-475 | Y | — | — | G | H | T | E | E | H | Y | V | K | — | M | 0.05 | 0.62 | 0.07 | 753 |
| Phage-476 | Y | — | M | G | H | S | H | E | H | I | W | K | — | — | 0.06 | 0.87 | 0.07 | 754 |
| Phage-477 | Y | — | — | G | H | S | H | E | Y | L | A | K | — | E | 0.05 | 0.86 | 0.06 | 755 |
| Phage-1334 | T | — | S | — | — | — | T | W | P | — | — | I | — | — | 0.08 | 2.00 | 0.07 | 794 |
| Phage-1335 | L | — | — | — | — | — | — | W | P | — | — | V | — | — | 0.06 | 2.16 | 0.08 | 795 |
| Phage-1336 | T | — | S | — | — | — | — | — | P | — | — | L | — | — | 0.07 | 2.38 | 0.06 | 796 |
| Phage-1337 | T | — | — | — | — | — | A | W | P | — | — | Q | — | — | 0.06 | 2.23 | 0.08 | 797 |
| Phage-1338 | T | — | S | — | — | — | T | — | T | — | — | K | — | F | 0.06 | 1.87 | 0.06 | 798 |
| Phage-1339 | T | — | — | — | — | — | T | W | S | — | — | A | — | F | 0.07 | 2.23 | 0.07 | 799 |
| Phage-1340 | T | — | — | — | — | — | A | W | G | — | — | L | — | F | 0.08 | 2.30 | 0.07 | 800 |
| Phage-1341 | L | — | — | — | — | — | T | — | P | — | — | L | — | — | 0.05 | 2.39 | 0.07 | 801 |
| Phage-1342 | T | — | S | — | — | — | A | — | P | — | — | L | — | — | 0.09 | 2.36 | 0.08 | 802 |
| Phage-1343 | T | — | S | — | — | — | A | — | S | — | — | A | — | F | 0.07 | 1.89 | 0.15 | 803 |
| Phage-1344 | L | — | — | — | — | — | T | — | P | — | — | T | — | — | 0.06 | 1.71 | 0.06 | 804 |
| Phage-1345 | — | — | S | — | — | — | H | — | P | — | — | Q | — | F | 0.06 | 1.16 | 0.06 | 805 |
| Phage-1346 | T | — | S | — | — | — | T | — | E | — | — | R | — | — | 0.06 | 1.84 | 0.06 | 806 |
| Phage-1347 | T | — | S | — | — | — | H | — | P | — | — | I | — | — | 0.06 | 1.78 | 0.07 | 807 |
| Phage-1348 | — | — | S | — | — | — | A | — | Y | — | — | W | — | — | 0.09 | 1.90 | 0.09 | 808 |
| Phage-1349 | T | — | S | — | — | — | T | W | — | — | — | L | — | F | 0.06 | 2.31 | 0.09 | 809 |
| Phage-1350 | T | — | — | — | — | — | T | — | L | — | — | T | — | — | 0.06 | 1.68 | 0.06 | 810 |
| Phage-1351 | T | — | — | — | — | — | H | — | P | — | — | A | — | — | 0.10 | 1.92 | 0.07 | 811 |
| Phage-1352 | L | — | — | — | — | F | T | — | P | — | — | L | — | — | 0.05 | 1.73 | 0.21 | 812 |
| Phage-1353 | L | — | K | P | — | V | P | — | K | — | — | T | — | — | 0.06 | 1.86 | 0.07 | 813 |
| Phage-1354 | T | — | S | — | — | — | T | W | P | — | — | I | — | — | 0.09 | 2.41 | 0.11 | 1650 |
| Phage-1355 | L | — | S | — | — | — | M | W | P | — | — | T | — | — | 0.08 | 2.39 | 0.08 | 1651 |
| Phage-1356 | L | — | S | — | — | — | M | — | S | — | — | T | — | — | 0.07 | 2.39 | 0.12 | 1652 |
| Phage-1357 | T | — | S | — | — | — | T | W | P | — | — | I | — | — | 0.06 | 2.35 | 0.08 | 1653 |
| Phage-1358 | T | — | — | — | — | — | M | W | — | — | — | V | — | — | 0.10 | 2.32 | 0.09 | 1654 |
| Phage-1359 | M | — | S | — | — | — | M | — | K | — | — | L | — | — | 0.05 | 2.32 | 0.05 | 1655 |
| Phage-1360 | T | — | S | — | — | — | T | — | — | — | — | F | — | — | 0.06 | 2.31 | 0.06 | 1656 |
| Phage-1361 | M | — | — | — | — | — | T | — | P | — | — | A | — | — | 0.07 | 2.30 | 0.06 | 1657 |
| Phage-1362 | — | — | — | — | — | — | M | W | P | — | — | V | — | V | 0.07 | 2.29 | 0.13 | 1658 |
| Phage-1363 | T | — | S | — | — | — | — | — | P | — | — | L | — | — | 0.06 | 2.29 | 0.08 | 1659 |
| Phage-1364 | — | — | K | P | — | V | A | N | Y | — | — | W | — | — | 0.08 | 2.29 | 0.10 | 1660 |
| Phage-1365 | M | — | S | — | — | — | M | — | S | — | — | L | — | — | 0.10 | 2.29 | 0.08 | 1661 |
| Phage-1366 | L | — | — | — | — | — | T | — | P | — | — | F | — | — | 0.06 | 2.28 | 0.07 | 1662 |
| Phage-1367 | M | — | — | — | — | — | N | — | P | — | — | Q | — | — | 0.06 | 2.28 | 0.08 | 1663 |
| Phage-1368 | T | — | S | — | — | — | T | W | S | — | — | M | — | — | 0.07 | 2.26 | 0.09 | 1664 |
| Phage-1369 | L | — | S | — | — | — | M | — | P | — | — | T | — | — | 0.09 | 2.25 | 0.07 | 1665 |
| Phage-1370 | T | — | — | — | — | — | H | — | S | — | — | A | — | F | 0.14 | 2.25 | 0.07 | 1666 |
| Phage-1371 | T | — | — | — | — | — | T | — | P | — | — | S | — | — | 0.06 | 2.25 | 0.06 | 1667 |
| Phage-1372 | R | — | — | — | — | — | — | — | P | — | — | P | — | — | 0.06 | 2.25 | 0.05 | 1668 |
| Phage-1373 | T | — | K | — | — | — | T | — | L | — | — | F | — | — | 0.08 | 2.23 | 0.08 | 1669 |
| Phage-1374 | M | — | — | — | — | F | H | — | P | — | — | A | — | — | 0.06 | 2.23 | 0.10 | 1670 |
| Phage-1375 | T | — | — | — | — | — | N | W | E | — | — | W | — | F | 0.08 | 2.22 | 0.08 | 1671 |
| Phage-1376 | T | — | — | — | — | — | T | — | — | — | — | — | — | V | 0.10 | 2.21 | 0.09 | 1672 |
| Phage-1377 | T | — | — | — | — | — | A | — | P | — | — | L | — | F | 0.12 | 2.18 | 0.11 | 1673 |
| Phage-1378 | T | — | S | — | — | — | — | N | K | L | S | L | — | — | 0.05 | 2.18 | 0.06 | 1674 |
| Phage-1379 | M | — | S | — | — | — | N | W | P | — | — | L | — | F | 0.06 | 2.18 | 0.07 | 1675 |
| Phage-1380 | R | — | S | — | — | F | H | — | P | L | S | L | — | — | 0.08 | 2.18 | 0.06 | 1676 |
| Phage-1381 | N | — | S | — | — | — | T | — | P | — | — | L | — | — | 0.06 | 2.17 | 0.07 | 1677 |
| Phage-1382 | — | — | — | — | — | — | M | — | S | — | — | T | — | — | 0.08 | 2.16 | 0.08 | 1678 |
| Phage-1383 | N | — | S | — | — | — | Q | W | P | — | — | I | — | — | 0.07 | 2.15 | 0.08 | 1679 |
| Phage-1384 | L | — | — | — | — | — | H | — | P | — | — | Q | — | — | 0.07 | 2.15 | 0.07 | 1680 |
| Phage-1385 | T | — | — | — | — | — | H | W | P | — | — | T | — | F | 0.08 | 2.15 | 0.07 | 1681 |
| Phage-1386 | T | — | S | — | — | — | T | — | T | — | S | K | — | F | 0.05 | 2.15 | 0.06 | 1682 |
| Phage-1387 | L | — | N | — | — | — | T | — | S | — | — | T | — | — | 0.05 | 2.14 | 0.06 | 1683 |
| Phage-1388 | T | — | S | — | — | — | T | — | T | — | — | W | — | F | 0.08 | 2.14 | 0.07 | 1684 |
| Phage-1389 | W | — | K | P | — | — | P | — | K | — | — | P | — | F | 0.05 | 2.14 | 0.07 | 1685 |
| Phage-1390 | T | — | — | — | — | — | M | — | T | — | — | L | — | F | 0.06 | 2.14 | 0.09 | 1686 |
| Phage-1391 | T | — | S | — | — | — | T | W | T | — | — | Q | — | — | 0.07 | 2.14 | 0.08 | 1687 |
| Phage-1392 | L | — | — | — | — | — | T | — | — | — | — | L | — | — | 0.08 | 2.13 | 0.07 | 1688 |
| Phage-1393 | M | — | — | E | F | F | N | — | P | — | — | L | — | — | 0.07 | 2.11 | 0.07 | 1689 |
| Phage-1394 | T | — | — | — | — | — | T | — | E | — | — | A | — | — | 0.05 | 2.09 | 0.07 | 1690 |
| Phage-1395 | T | — | S | — | — | — | Y | — | T | — | — | — | — | F | 0.07 | 2.09 | 0.08 | 1691 |
| Phage-1396 | T | — | — | — | V | F | — | — | G | — | — | E | — | F | 0.06 | 2.09 | 0.07 | 1692 |
| Phage-1397 | — | — | S | — | — | — | M | — | T | — | — | T | — | — | 0.09 | 2.09 | 0.06 | 1693 |
| Phage-1398 | Q | — | — | — | — | — | — | — | P | — | — | M | — | — | 0.07 | 2.08 | 0.08 | 1694 |
| Phage-1399 | Y | — | — | — | — | — | T | — | P | — | — | L | — | — | 0.06 | 2.08 | 0.08 | 1695 |
| Phage-1400 | T | — | — | — | — | — | T | — | — | — | — | E | — | — | 0.05 | 2.08 | 0.11 | 1696 |
| Phage-1401 | T | — | S | E | — | — | T | — | T | — | — | W | — | F | 0.06 | 2.05 | 0.06 | 1697 |
| Phage-1402 | T | — | — | — | — | — | H | — | L | — | — | W | — | M | 0.10 | 2.05 | 0.08 | 1698 |
| Phage-1403 | T | — | S | — | — | — | T | — | V | — | — | Q | — | F | 0.08 | 2.05 | 0.07 | 1699 |
| Phage-1404 | G | — | — | — | — | — | T | — | P | — | — | S | — | — | 0.06 | 2.05 | 0.06 | 1700 |
| Phage-1405 | T | — | — | — | — | — | T | — | K | — | — | A | — | — | 0.07 | 2.05 | 0.09 | 1701 |
| Phage-1406 | M | — | S | — | — | — | M | — | P | — | — | T | — | — | 0.06 | 2.04 | 0.08 | 1702 |
| Phage-1407 | T | — | — | — | — | — | — | — | — | — | — | W | — | — | 0.07 | 2.03 | 0.08 | 1703 |
| Phage-1408 | T | — | S | — | — | — | — | W | — | — | — | W | — | F | 0.07 | 2.03 | 0.08 | 1704 |
| Phage-1409 | T | — | — | — | — | — | M | W | H | — | — | V | — | — | 0.07 | 2.03 | 0.09 | 1705 |
| Phage-1410 | T | — | S | — | — | F | H | — | E | — | — | R | — | — | 0.05 | 2.02 | 0.05 | 1706 |
| Phage-1411 | L | — | — | — | — | F | K | — | P | — | — | L | — | — | 0.07 | 2.02 | 0.07 | 1707 |
| Phage-1412 | L | — | E | E | F | — | D | — | P | — | — | M | — | — | 0.05 | 2.01 | 0.06 | 1708 |
| Phage-1413 | T | — | — | — | — | — | T | — | S | — | — | M | — | F | 0.06 | 2.00 | 0.07 | 1709 |
| Phage-1414 | L | — | — | — | — | — | — | — | P | — | — | T | — | — | 0.09 | 2.00 | 0.10 | 1710 |
| Phage-1415 | H | — | — | — | — | — | H | — | P | — | — | A | — | — | 0.07 | 2.00 | 0.12 | 1711 |
| Phage-1416 | T | — | — | — | — | — | N | — | P | — | — | — | — | — | 0.07 | 1.99 | 0.14 | 1712 |
| Phage-1417 | M | — | — | — | — | — | T | — | P | — | — | L | — | — | 0.06 | 1.99 | 0.07 | 1713 |
| Phage-1418 | T | — | — | — | — | — | T | — | S | — | — | W | — | F | 0.09 | 1.99 | 0.07 | 1714 |
| Phage-1419 | T | — | — | — | — | — | H | — | T | — | — | E | — | F | 0.07 | 1.99 | 0.07 | 1715 |
| Phage-1420 | — | — | — | — | V | F | — | — | S | — | — | M | — | F | 0.06 | 1.99 | 0.06 | 1716 |
| Phage-1421 | — | — | — | — | — | — | T | — | S | — | — | M | — | — | 0.07 | 1.99 | 0.09 | 1717 |
| Phage-1422 | L | — | — | E | F | F | N | — | P | — | — | L | — | — | 0.06 | 1.98 | 0.06 | 1718 |
| Phage-1423 | Q | — | S | — | — | — | A | W | P | — | — | F | — | — | 0.07 | 1.97 | 0.08 | 1719 |
| Phage-1424 | M | — | — | — | — | — | H | — | T | — | — | T | — | — | 0.10 | 1.97 | 0.07 | 1720 |
| Phage-1425 | T | — | S | — | — | — | T | — | E | — | — | W | — | M | 0.06 | 1.97 | 0.07 | 1721 |
| Phage-1426 | M | — | S | — | — | — | N | — | P | — | — | W | — | — | 0.07 | 1.97 | 0.08 | 1722 |
| Phage-1427 | T | — | — | — | — | — | T | — | E | — | — | — | — | F | 0.08 | 1.96 | 0.06 | 1723 |
| Phage-1428 | W | — | — | — | V | F | — | — | T | — | S | P | — | — | 0.06 | 1.96 | 0.07 | 1724 |
| Phage-1429 | T | — | — | — | — | — | A | — | T | — | — | A | — | F | 0.06 | 1.96 | 0.06 | 1725 |
| Phage-1430 | T | — | — | — | — | — | T | — | E | — | — | T | — | — | 0.08 | 1.96 | 0.08 | 1726 |
| Phage-1431 | L | — | K | P | — | V | P | N | S | — | — | T | — | — | 0.06 | 1.95 | 0.06 | 1727 |
| Phage-1432 | T | — | S | — | — | — | H | — | P | — | — | D | — | I | 0.06 | 1.95 | 0.07 | 1728 |
| Phage-1433 | P | — | — | — | — | — | M | W | P | — | — | V | — | — | 0.05 | 1.95 | 0.06 | 1729 |
| Phage-1434 | L | — | N | — | — | — | M | — | K | — | — | T | — | — | 0.07 | 1.94 | 0.06 | 1730 |
| Phage-1435 | M | — | S | — | — | — | M | — | P | — | — | L | — | — | 0.07 | 1.93 | 0.07 | 1731 |
| Phage-1436 | T | — | S | — | — | — | M | W | T | — | — | — | — | F | 0.09 | 1.93 | 0.08 | 1732 |
| Phage-1437 | T | — | — | — | — | — | Y | W | T | — | — | A | — | F | 0.09 | 1.93 | 0.09 | 1733 |
| Phage-1438 | T | — | S | — | — | — | G | — | P | — | — | L | — | Y | 0.07 | 1.92 | 0.08 | 1734 |
| Phage-1439 | — | — | S | — | — | — | — | L | Q | L | S | I | — | — | 0.05 | 1.92 | 0.06 | 1735 |
| Phage-1440 | T | — | S | — | — | — | T | W | — | — | — | S | — | F | 0.09 | 1.92 | 0.10 | 1736 |
| Phage-1441 | T | — | S | — | — | — | H | — | V | — | — | M | — | F | 0.07 | 1.91 | 0.17 | 1737 |
| Phage-1442 | T | — | S | — | — | — | T | — | I | — | — | M | — | F | 0.05 | 1.90 | 0.08 | 1738 |
| Phage-1443 | T | — | R | — | — | — | T | — | N | — | — | F | — | — | 0.05 | 1.90 | 0.07 | 1739 |
| Phage-1444 | T | — | S | — | — | — | T | W | P | — | — | I | — | — | 0.07 | 1.89 | 0.08 | 1740 |
| Phage-1445 | T | — | — | — | — | — | — | — | P | — | — | A | — | — | 0.10 | 1.89 | 0.09 | 1741 |
| Phage-1446 | — | — | — | — | V | F | — | F | P | — | S | P | — | — | 0.06 | 1.87 | 0.08 | 1742 |
| Phage-1447 | T | — | S | — | — | F | — | — | S | — | — | A | — | F | 0.05 | 1.86 | 0.07 | 1743 |
| Phage-1448 | T | — | T | — | — | F | — | — | N | — | — | M | — | F | 0.06 | 1.85 | 0.06 | 1744 |
| Phage-1449 | T | — | S | — | — | — | T | — | — | — | — | A | — | Y | 0.11 | 1.84 | 0.14 | 1745 |
| Phage-1450 | T | — | S | — | — | — | T | — | S | — | — | Q | — | — | 0.06 | 1.84 | 0.07 | 1746 |
| Phage-1451 | T | — | S | — | — | — | Y | — | — | — | — | V | — | — | 0.09 | 1.84 | 0.08 | 1747 |
| Phage-1452 | L | — | R | — | — | — | M | W | P | — | — | T | — | — | 0.07 | 1.83 | 0.06 | 1748 |
| Phage-1453 | T | — | S | — | — | — | T | F | H | — | — | Q | — | F | 0.07 | 1.83 | 0.08 | 1749 |
| Phage-1454 | T | — | S | — | — | — | H | — | E | — | — | — | — | — | 0.08 | 1.82 | 0.11 | 1750 |
| Phage-1455 | Q | — | — | — | — | — | — | — | P | — | — | V | — | — | 0.06 | 1.82 | 0.07 | 1751 |
| Phage-1456 | L | — | K | P | — | V | P | — | Q | — | — | T | — | F | 0.06 | 1.82 | 0.05 | 1752 |
| Phage-1457 | T | — | — | — | — | — | M | W | Q | — | — | V | — | — | 0.06 | 1.81 | 0.06 | 1753 |
| Phage-1458 | P | — | S | — | — | — | T | — | — | — | L | — | — | V | 0.06 | 1.81 | 0.06 | 1754 |
| Phage-1459 | — | — | — | — | — | — | M | W | P | — | — | V | — | V | 0.05 | 1.80 | 0.06 | 1755 |
| Phage-1460 | — | — | — | — | — | — | A | — | P | — | — | A | — | — | 0.06 | 1.80 | 0.06 | 1756 |
| Phage-1461 | Y | — | S | — | — | — | Q | W | P | — | — | I | — | — | 0.09 | 1.79 | 0.08 | 1757 |
| Phage-1462 | T | — | — | — | — | — | — | — | Q | — | — | L | — | F | 0.07 | 1.79 | 0.07 | 1758 |
| Phage-1463 | — | — | — | — | — | — | — | — | S | — | — | A | — | F | 0.06 | 1.79 | 0.07 | 1759 |
| Phage-1464 | — | — | S | — | — | F | H | — | P | — | — | I | — | — | 0.05 | 1.79 | 0.07 | 1760 |
| Phage-1465 | L | — | K | P | — | V | P | — | Q | — | — | T | — | — | 0.06 | 1.78 | 0.07 | 1761 |
| Phage-1466 | M | — | T | — | — | — | L | — | N | — | — | T | — | — | 0.06 | 1.78 | 0.09 | 1762 |
| Phage-1467 | T | — | — | — | — | — | H | — | P | — | — | — | — | — | 0.06 | 1.77 | 0.05 | 1763 |
| Phage-1468 | W | — | — | E | F | F | N | — | H | — | — | L | — | F | 0.07 | 1.77 | 0.06 | 1764 |
| Phage-1469 | T | — | S | — | — | F | — | L | Q | L | S | I | — | — | 0.06 | 1.77 | 0.06 | 1765 |
| Phage-1470 | — | — | — | — | — | — | — | — | P | — | — | Q | — | — | 0.07 | 1.77 | 0.11 | 1766 |
| Phage-1471 | L | — | T | — | — | L | A | — | N | — | — | T | — | — | 0.05 | 1.77 | 0.05 | 1767 |
| Phage-1472 | G | — | — | — | — | — | H | — | P | — | — | F | — | — | 0.06 | 1.76 | 0.06 | 1768 |
| Phage-1473 | T | — | — | — | — | — | V | W | E | — | — | M | — | F | 0.07 | 1.76 | 0.07 | 1769 |
| Phage-1474 | T | — | — | — | — | — | N | — | P | — | — | L | — | — | 0.05 | 1.75 | 0.08 | 1770 |
| Phage-1475 | M | — | — | — | — | — | M | — | — | — | — | T | — | — | 0.06 | 1.75 | 0.09 | 1771 |
| Phage-1476 | T | — | — | — | — | — | — | F | P | — | — | W | — | — | 0.06 | 1.75 | 0.06 | 1772 |
| Phage-1477 | — | — | — | — | V | F | P | — | P | — | — | P | — | F | 0.05 | 1.75 | 0.06 | 1773 |
| Phage-1478 | T | — | S | — | — | — | M | — | P | — | — | W | — | V | 0.06 | 1.75 | 0.08 | 1774 |
| Phage-1479 | W | — | — | — | V | F | — | — | S | — | — | L | — | — | 0.05 | 1.75 | 0.06 | 1775 |
| Phage-1480 | G | — | — | — | — | F | H | — | P | — | — | L | — | — | 0.05 | 1.74 | 0.08 | 1776 |
| Phage-1481 | L | — | E | E | F | F | H | — | S | — | — | V | — | — | 0.06 | 1.74 | 0.06 | 1777 |
| Phage-1482 | T | — | — | — | — | — | A | W | P | — | — | H | — | — | 0.05 | 1.73 | 0.06 | 1778 |
| Phage-1483 | Q | — | — | — | — | — | M | W | T | — | — | A | — | F | 0.06 | 1.73 | 0.07 | 1779 |
| Phage-1484 | M | — | S | — | — | — | T | — | P | — | — | M | — | — | 0.07 | 1.73 | 0.06 | 1780 |
| Phage-1485 | T | — | — | — | — | — | T | — | P | — | — | S | — | — | 0.06 | 1.73 | 0.05 | 1781 |
| Phage-1486 | T | — | — | — | — | — | V | W | G | — | — | L | — | F | 0.06 | 1.73 | 0.06 | 1782 |
| Phage-1487 | T | — | — | — | — | — | H | — | — | — | — | E | — | F | 0.06 | 1.72 | 0.06 | 1783 |
| Phage-1488 | R | — | R | — | — | F | — | — | N | — | — | — | — | M | 0.05 | 1.72 | 0.06 | 1784 |
| Phage-1489 | W | — | — | — | — | — | T | W | P | — | — | L | — | I | 0.14 | 1.70 | 0.17 | 1785 |
| Phage-1490 | R | — | — | — | — | V | M | — | D | — | — | V | — | — | 0.07 | 1.70 | 0.07 | 1786 |
| Phage-1491 | T | — | S | — | — | — | H | W | T | — | — | L | — | F | 0.06 | 1.69 | 0.06 | 1787 |
| Phage-1492 | T | — | S | — | — | — | H | F | — | — | — | M | — | F | 0.06 | 1.69 | 0.07 | 1788 |
| Phage-1493 | — | — | — | — | — | — | H | — | V | — | — | F | — | F | 0.07 | 1.68 | 0.08 | 1789 |
| Phage-1494 | T | — | S | — | — | — | T | — | N | — | — | W | — | F | 0.06 | 1.68 | 0.05 | 1790 |
| Phage-1495 | T | — | S | — | — | — | — | — | P | — | — | V | — | — | 0.06 | 1.67 | 0.07 | 1791 |
| Phage-1496 | W | — | — | — | — | — | M | — | P | — | — | L | — | — | 0.08 | 1.67 | 0.06 | 1792 |
| Phage-1497 | L | — | N | — | — | — | P | — | K | — | — | T | — | — | 0.06 | 1.66 | 0.07 | 1793 |
| Phage-1498 | L | — | S | — | — | — | M | — | P | — | — | L | — | Y | 0.06 | 1.66 | 0.08 | 1794 |
| Phage-1499 | T | — | — | — | — | — | M | W | V | — | — | T | — | — | 0.06 | 1.66 | 0.07 | 1795 |
| Phage-1500 | L | — | — | — | — | — | N | — | P | — | — | A | — | — | 0.06 | 1.66 | 0.07 | 1796 |
| Phage-1501 | — | — | — | — | — | — | T | — | T | — | — | M | — | I | 0.07 | 1.66 | 0.07 | 1797 |
| Phage-1502 | — | — | — | — | — | L | — | — | P | — | — | D | — | — | 0.05 | 1.65 | 0.07 | 1798 |
| Phage-1503 | T | — | — | — | — | — | G | W | — | — | — | F | — | F | 0.07 | 1.65 | 0.09 | 1799 |
| Phage-1504 | M | — | S | — | — | — | T | — | P | — | — | — | — | — | 0.06 | 1.64 | 0.10 | 1800 |
| Phage-1505 | L | — | S | — | — | — | T | — | P | — | — | W | — | M | 0.06 | 1.64 | 0.06 | 1801 |
| Phage-1506 | D | — | W | G | D | — | P | — | P | — | R | V | — | — | 0.06 | 1.64 | 0.07 | 1802 |
| Phage-1507 | W | — | T | — | — | F | — | — | K | L | S | I | — | — | 0.06 | 1.63 | 0.06 | 1803 |
| Phage-1508 | L | — | S | — | — | — | M | — | Q | — | — | T | — | — | 0.06 | 1.63 | 0.06 | 1804 |
| Phage-1509 | T | — | S | — | — | — | N | — | E | — | — | W | — | F | 0.06 | 1.62 | 0.06 | 1805 |
| Phage-1510 | T | — | — | — | — | — | H | — | E | — | — | M | — | Y | 0.08 | 1.61 | 0.12 | 1806 |
| Phage-1511 | — | — | S | — | V | F | — | — | — | — | — | T | — | — | 0.05 | 1.61 | 0.08 | 1807 |
| Phage-1512 | T | — | — | — | — | — | T | W | P | — | — | S | — | — | 0.06 | 1.61 | 0.08 | 1808 |
| Phage-1513 | T | — | — | — | — | — | H | W | I | — | — | W | — | E | 0.07 | 1.61 | 0.07 | 1809 |
| Phage-1514 | T | — | — | — | — | — | G | W | Q | — | — | M | — | F | 0.10 | 1.60 | 0.11 | 1810 |
| Phage-1515 | — | — | — | — | — | — | T | — | E | — | — | R | — | — | 0.06 | 1.60 | 0.07 | 1811 |
| Phage-1516 | G | — | — | — | — | — | N | — | P | — | — | L | — | — | 0.06 | 1.59 | 0.05 | 1812 |
| Phage-1517 | T | — | — | — | — | — | Y | — | — | — | — | M | — | — | 0.06 | 1.59 | 0.07 | 1813 |
| Phage-1518 | — | — | — | — | — | — | P | — | P | — | — | E | — | — | 0.06 | 1.59 | 0.07 | 1814 |
| Phage-1519 | T | — | S | — | — | — | A | — | — | — | — | — | — | — | 0.10 | 1.59 | 0.07 | 1815 |
| Phage-1520 | L | — | — | — | — | — | A | — | P | — | — | M | — | — | 0.06 | 1.59 | 0.06 | 1816 |
| Phage-1521 | — | — | — | — | — | — | T | — | E | — | — | I | — | — | 0.05 | 1.59 | 0.05 | 1817 |
| Phage-1522 | — | — | — | — | — | — | M | W | P | — | — | V | — | V | 0.05 | 1.59 | 0.07 | 1818 |
| Phage-1523 | — | — | — | — | — | — | Q | — | G | — | — | M | — | — | 0.07 | 1.59 | 0.05 | 1819 |
| Phage-1524 | W | — | — | — | — | — | — | — | P | — | — | W | — | — | 0.06 | 1.58 | 0.05 | 1820 |
| Phage-1525 | T | — | T | — | V | F | P | — | E | — | — | R | — | — | 0.06 | 1.57 | 0.07 | 1821 |
| Phage-1526 | — | — | — | — | — | — | T | F | H | — | — | M | — | F | 0.07 | 1.57 | 0.09 | 1822 |
| Phage-1527 | T | — | S | — | — | F | — | — | K | — | — | W | — | — | 0.05 | 1.57 | 0.06 | 1823 |
| Phage-1528 | T | — | — | — | — | — | M | W | T | — | — | E | — | — | 0.07 | 1.57 | 0.07 | 1824 |
| Phage-1529 | T | — | — | — | — | — | T | — | P | — | — | E | — | M | 0.05 | 1.57 | 0.06 | 1825 |
| Phage-1530 | L | — | — | — | — | F | N | — | P | — | — | L | — | — | 0.05 | 1.57 | 0.05 | 1826 |
| Phage-1531 | M | — | T | — | — | — | T | — | P | — | — | A | — | — | 0.08 | 1.56 | 0.07 | 1827 |
| Phage-1532 | — | — | S | — | — | — | A | W | P | — | — | W | — | — | 0.09 | 1.55 | 0.08 | 1828 |
| Phage-1533 | L | — | — | E | — | F | D | — | S | — | — | M | — | — | 0.06 | 1.55 | 0.08 | 1829 |
| Phage-1534 | T | — | S | — | — | — | T | — | S | — | — | F | — | F | 0.07 | 1.55 | 0.07 | 1830 |
| Phage-1535 | — | — | K | — | — | — | P | — | Y | — | — | G | — | F | 0.07 | 1.54 | 0.08 | 1831 |
| Phage-1536 | T | — | S | — | V | F | — | — | T | — | S | N | — | F | 0.05 | 1.54 | 0.06 | 1832 |
| Phage-1537 | Q | — | — | — | — | — | M | W | T | — | — | A | — | F | 0.06 | 1.54 | 0.06 | 1833 |
| Phage-1538 | T | — | — | — | — | — | A | W | E | — | — | I | — | F | 0.06 | 1.54 | 0.06 | 1834 |
| Phage-1539 | T | — | — | — | — | — | M | W | N | — | — | Q | — | Y | 0.07 | 1.54 | 0.07 | 1835 |
| Phage-1540 | — | — | S | — | — | — | M | — | P | — | — | M | — | — | 0.06 | 1.53 | 0.06 | 1836 |
| Phage-1541 | T | — | — | — | — | — | — | W | E | — | — | L | — | F | 0.06 | 1.52 | 0.08 | 1837 |
| Phage-1542 | T | — | S | — | — | — | T | — | V | — | — | D | — | F | 0.06 | 1.52 | 0.09 | 1838 |
| Phage-1543 | T | — | E | — | V | F | I | — | S | — | — | F | — | — | 0.09 | 1.52 | 0.08 | 1839 |
| Phage-1544 | L | — | S | — | — | — | M | W | Q | — | — | T | — | — | 0.05 | 1.51 | 0.05 | 1840 |
| Phage-1545 | L | — | — | E | — | — | D | — | P | — | — | M | — | — | 0.06 | 1.51 | 0.08 | 1841 |
| Phage-1546 | T | — | — | — | — | — | T | W | D | — | — | L | — | F | 0.07 | 1.51 | 0.06 | 1842 |
| Phage-1547 | — | — | — | — | — | — | H | — | K | — | — | V | — | I | 0.06 | 1.51 | 0.06 | 1843 |
| Phage-1548 | I | — | — | — | — | F | I | W | S | — | — | A | — | F | 0.08 | 1.51 | 0.08 | 1844 |
| Phage-1549 | T | — | — | — | — | — | T | — | S | — | — | D | — | F | 0.08 | 1.51 | 0.06 | 1845 |
| Phage-1550 | Q | — | — | — | — | — | M | W | T | — | — | V | — | F | 0.09 | 1.50 | 0.07 | 1846 |
| Phage-1551 | T | — | — | — | — | — | H | — | L | — | — | H | — | — | 0.07 | 1.50 | 0.12 | 1847 |
| Phage-1552 | P | — | — | — | — | — | M | W | — | — | — | — | — | — | 0.06 | 1.49 | 0.05 | 1848 |
| Phage-1553 | N | — | S | — | — | — | T | W | P | — | — | M | — | F | 0.08 | 1.49 | 0.10 | 1849 |
| Phage-1554 | T | — | — | — | — | V | H | F | G | — | — | L | — | F | 0.06 | 1.49 | 0.07 | 1850 |
| Phage-1555 | T | — | — | — | — | — | T | — | T | — | — | T | — | F | 0.05 | 1.48 | 0.05 | 1851 |
| Phage-1556 | T | — | — | — | — | — | — | W | E | — | — | A | — | — | 0.05 | 1.48 | 0.08 | 1852 |
| Phage-1557 | T | — | — | — | — | — | M | — | Q | — | — | E | — | F | 0.05 | 1.48 | 0.06 | 1853 |
| Phage-1558 | T | — | — | — | — | — | H | — | T | — | — | T | — | — | 0.06 | 1.47 | 0.07 | 1854 |
| Phage-1559 | T | — | S | — | — | — | T | — | K | — | — | V | — | — | 0.06 | 1.47 | 0.06 | 1855 |
| Phage-1560 | — | — | S | — | — | — | T | — | E | — | — | — | — | F | 0.06 | 1.47 | 0.06 | 1856 |
| Phage-1561 | T | — | — | — | — | — | N | W | H | — | — | W | — | F | 0.08 | 1.46 | 0.15 | 1857 |
| Phage-1562 | L | — | — | — | — | — | T | — | P | — | — | I | — | — | 0.05 | 1.46 | 0.08 | 1858 |
| Phage-1563 | — | — | S | — | — | — | L | W | K | — | — | V | — | F | 0.06 | 1.45 | 0.07 | 1859 |
| Phage-1564 | T | — | S | — | — | F | — | — | K | — | — | W | — | W | 0.05 | 1.45 | 0.06 | 1860 |
| Phage-1565 | — | — | S | — | — | — | M | — | Q | — | — | L | — | F | 0.06 | 1.44 | 0.09 | 1861 |
| Phage-1566 | Q | — | — | — | — | — | M | W | T | — | — | A | — | F | 0.06 | 1.44 | 0.06 | 1862 |
| Phage-1567 | T | — | S | — | — | — | T | — | M | — | — | A | — | F | 0.09 | 1.44 | 0.10 | 1863 |
| Phage-1568 | T | — | S | — | — | — | T | — | — | — | — | A | — | F | 0.06 | 1.44 | 0.08 | 1864 |
| Phage-1569 | W | — | G | V | — | M | D | W | R | — | — | E | — | — | 0.06 | 1.44 | 0.16 | 1865 |
| Phage-1570 | T | — | E | D | — | F | H | F | S | — | — | E | — | F | 0.08 | 1.44 | 0.07 | 1866 |
| Phage-1571 | Q | — | — | — | — | — | M | W | T | — | — | A | — | F | 0.05 | 1.42 | 0.07 | 1867 |
| Phage-1572 | T | — | S | — | — | — | T | — | T | — | — | S | — | F | 0.07 | 1.42 | 0.06 | 1868 |
| Phage-1573 | W | — | D | Q | F | F | H | — | K | — | — | L | — | — | 0.05 | 1.42 | 0.07 | 1869 |
| Phage-1574 | — | — | — | — | — | — | T | — | V | — | — | M | — | F | 0.06 | 1.42 | 0.07 | 1870 |
| Phage-1575 | T | — | — | — | — | — | H | — | S | — | — | F | — | M | 0.07 | 1.42 | 0.07 | 1871 |
| Phage-1576 | — | — | S | — | — | — | — | L | Q | L | S | Q | — | F | 0.06 | 1.41 | 0.05 | 1872 |
| Phage-1577 | R | — | S | F | R | F | I | T | L | A | E | A | — | V | 0.05 | 1.41 | 0.08 | 1873 |
| Phage-1578 | T | — | S | — | — | — | H | — | P | — | — | I | — | — | 0.06 | 1.41 | 0.08 | 1874 |
| Phage-1579 | T | — | — | — | — | — | Q | F | — | — | — | L | — | — | 0.06 | 1.41 | 0.07 | 1875 |
| Phage-1580 | L | — | S | — | — | F | L | F | Q | — | S | T | — | — | 0.05 | 1.41 | 0.06 | 1876 |
| Phage-1581 | R | — | — | — | — | F | H | — | S | — | — | N | — | F | 0.05 | 1.41 | 0.06 | 1877 |
| Phage-1582 | G | — | — | — | — | — | — | — | P | — | — | A | — | — | 0.05 | 1.41 | 0.06 | 1878 |
| Phage-1583 | P | — | — | — | — | — | M | W | P | — | — | V | — | — | 0.05 | 1.41 | 0.06 | 1879 |
| Phage-1584 | T | — | S | — | — | — | T | — | S | — | — | A | — | — | 0.05 | 1.41 | 0.06 | 1880 |
| Phage-1585 | T | — | — | — | — | — | — | W | N | — | — | A | — | E | 0.07 | 1.41 | 0.07 | 1881 |
| Phage-1586 | L | — | — | — | — | — | H | — | S | — | — | A | — | F | 0.09 | 1.41 | 0.09 | 1882 |
| Phage-1587 | P | — | S | — | — | V | T | — | — | — | L | — | — | V | 0.05 | 1.41 | 0.06 | 1883 |
| Phage-1588 | M | — | — | — | — | — | A | W | — | — | — | L | — | — | 0.06 | 1.40 | 0.07 | 1884 |
| Phage-1589 | T | — | — | — | — | — | — | — | P | — | — | L | — | V | 0.06 | 1.40 | 0.06 | 1885 |
| Phage-1590 | L | — | — | — | — | F | M | — | E | — | — | L | — | F | 0.05 | 1.40 | 0.06 | 1886 |
| Phage-1591 | T | — | S | — | — | — | E | — | T | — | — | L | — | F | 0.07 | 1.40 | 0.08 | 1887 |
| Phage-1592 | — | — | — | — | — | — | A | W | G | — | — | A | — | F | 0.06 | 1.39 | 0.09 | 1888 |
| Phage-1593 | T | — | — | — | — | — | T | W | L | — | — | V | — | — | 0.06 | 1.39 | 0.08 | 1889 |
| Phage-1594 | — | — | S | — | F | F | H | F | H | — | — | A | — | — | 0.08 | 1.38 | 0.06 | 1890 |
| Phage-1595 | T | — | S | — | — | — | N | W | V | — | — | L | — | — | 0.06 | 1.38 | 0.07 | 1891 |
| Phage-1596 | T | — | S | — | — | — | H | W | V | — | — | — | — | — | 0.07 | 1.37 | 0.09 | 1892 |
| Phage-1597 | — | — | — | — | — | — | T | — | G | — | — | E | — | F | 0.06 | 1.36 | 0.07 | 1893 |
| Phage-1598 | — | — | — | — | — | — | T | W | — | — | — | A | — | — | 0.06 | 1.36 | 0.06 | 1894 |
| Phage-1599 | L | — | S | — | — | — | M | W | E | — | — | L | — | — | 0.06 | 1.36 | 0.07 | 1895 |
| Phage-1600 | T | — | S | — | — | — | G | — | P | — | — | T | — | — | 0.06 | 1.36 | 0.06 | 1896 |
| Phage-1601 | T | — | S | — | — | — | A | W | T | — | — | L | — | F | 0.05 | 1.36 | 0.06 | 1897 |
| Phage-1602 | T | — | S | — | — | — | A | — | E | — | — | E | — | F | 0.06 | 1.36 | 0.06 | 1898 |
| Phage-1603 | T | — | S | — | — | — | L | W | E | — | — | M | — | F | 0.06 | 1.35 | 0.06 | 1899 |
| Phage-1604 | P | — | — | — | — | — | H | — | T | — | — | T | — | — | 0.07 | 1.34 | 0.07 | 1900 |
| Phage-1605 | — | — | S | — | — | — | T | — | T | — | — | A | — | — | 0.08 | 1.34 | 0.07 | 1901 |
| Phage-1606 | T | — | S | — | — | — | H | — | S | — | — | K | — | F | 0.06 | 1.33 | 0.07 | 1902 |
| Phage-1607 | — | — | — | — | — | — | H | — | P | — | — | E | — | — | 0.05 | 1.32 | 0.05 | 1903 |
| Phage-1608 | — | — | — | E | V | F | N | — | V | — | — | F | — | F | 0.07 | 1.32 | 0.07 | 1904 |
| Phage-1609 | T | — | — | — | — | — | A | — | T | — | — | V | — | F | 0.05 | 1.32 | 0.08 | 1905 |
| Phage-1610 | — | — | — | — | — | — | M | W | E | — | — | K | — | F | 0.07 | 1.32 | 0.09 | 1906 |
| Phage-1611 | — | — | — | D | — | — | I | W | E | — | — | W | — | F | 0.06 | 1.32 | 0.07 | 1907 |
| Phage-1612 | T | — | — | — | — | — | T | — | E | — | — | S | — | — | 0.06 | 1.32 | 0.09 | 1908 |
| Phage-1613 | — | — | S | — | — | — | M | — | T | — | — | T | — | — | 0.08 | 1.31 | 0.08 | 1909 |
| Phage-1614 | L | — | — | — | — | — | A | W | E | — | — | M | — | F | 0.08 | 1.30 | 0.06 | 1910 |
| Phage-1615 | Q | — | S | — | — | — | T | — | — | — | — | F | — | — | 0.07 | 1.29 | 0.08 | 1911 |
| Phage-1616 | T | — | — | — | — | V | P | — | K | — | — | P | — | — | 0.06 | 1.29 | 0.06 | 1912 |
| Phage-1617 | — | — | — | — | — | — | T | — | T | — | — | F | — | — | 0.07 | 1.29 | 0.07 | 1913 |
| Phage-1618 | R | — | — | — | V | F | — | — | T | — | — | P | — | — | 0.06 | 1.29 | 0.07 | 1914 |
| Phage-1619 | T | — | S | — | — | — | T | — | K | — | — | A | — | F | 0.07 | 1.29 | 0.05 | 1915 |
| Phage-1620 | L | — | — | — | — | — | Y | — | P | — | — | L | — | F | 0.06 | 1.28 | 0.06 | 1916 |
| Phage-1621 | T | — | T | — | — | F | — | — | K | — | — | T | — | — | 0.08 | 1.28 | 0.09 | 1917 |
| Phage-1622 | N | — | S | — | — | — | T | W | P | — | — | M | — | F | 0.05 | 1.27 | 0.06 | 1918 |
| Phage-1623 | — | — | S | — | — | — | T | — | Q | — | — | — | — | F | 0.08 | 1.26 | 0.06 | 1919 |
| Phage-1624 | — | — | — | — | — | — | T | — | S | — | — | A | — | — | 0.07 | 1.25 | 0.07 | 1920 |
| Phage-1625 | N | — | — | — | — | — | M | — | S | — | — | T | — | — | 0.06 | 1.25 | 0.07 | 1921 |
| Phage-1626 | R | — | — | — | — | — | M | W | T | — | — | A | — | F | 0.16 | 1.25 | 0.06 | 1922 |
| Phage-1627 | T | — | — | — | — | — | T | — | M | — | — | V | — | — | 0.05 | 1.24 | 0.07 | 1923 |
| Phage-1628 | — | — | — | — | — | — | T | W | K | — | — | E | — | — | 0.05 | 1.24 | 0.07 | 1924 |
| Phage-1629 | T | — | — | — | — | — | T | W | L | — | — | W | — | — | 0.06 | 1.23 | 0.07 | 1925 |
| Phage-1630 | T | — | — | — | — | — | V | W | T | — | — | I | — | F | 0.06 | 1.23 | 0.05 | 1926 |
| Phage-1631 | T | — | — | D | — | — | F | W | Q | — | — | V | — | — | 0.07 | 1.22 | 0.07 | 1927 |
| Phage-1632 | T | — | S | — | — | — | — | W | G | — | — | W | — | — | 0.06 | 1.22 | 0.07 | 1928 |
| Phage-1633 | T | — | S | — | — | — | T | — | S | — | — | — | — | — | 0.06 | 1.22 | 0.07 | 1929 |
| Phage-1634 | T | — | S | — | — | — | T | — | S | — | — | S | — | F | 0.07 | 1.21 | 0.09 | 1930 |
| Phage-1635 | L | — | — | — | — | — | T | — | P | — | — | D | — | V | 0.06 | 1.21 | 0.05 | 1931 |
| Phage-1636 | T | — | — | — | — | — | N | W | P | — | — | E | — | — | 0.07 | 1.21 | 0.08 | 1932 |
| Phage-1637 | L | — | E | E | F | F | H | — | S | — | — | L | — | — | 0.06 | 1.21 | 0.06 | 1933 |
| Phage-1638 | P | — | S | D | — | V | A | — | P | — | — | — | — | V | 0.07 | 1.20 | 0.06 | 1934 |
| Phage-1639 | — | — | — | — | — | — | H | W | S | — | — | V | — | F | 0.06 | 1.20 | 0.08 | 1935 |
| Phage-1640 | L | — | S | — | — | — | M | — | P | — | — | V | — | — | 0.06 | 1.19 | 0.08 | 1936 |
| Phage-1641 | — | — | S | — | — | — | T | W | — | — | — | Q | — | F | 0.06 | 1.19 | 0.08 | 1937 |
| Phage-1642 | — | — | S | — | V | F | — | — | R | — | — | K | — | — | 0.05 | 1.18 | 0.06 | 1938 |
| Phage-1643 | — | — | S | — | — | — | G | W | — | — | — | M | — | F | 0.06 | 1.16 | 0.08 | 1939 |
| Phage-1644 | — | — | — | — | — | F | D | — | G | — | — | M | — | — | 0.08 | 1.16 | 0.06 | 1940 |
| Phage-1645 | — | — | — | D | — | F | N | — | V | — | — | L | — | F | 0.06 | 1.14 | 0.08 | 1941 |
| Phage-1646 | T | — | — | — | — | — | N | — | S | — | — | A | — | — | 0.06 | 1.14 | 0.08 | 1942 |
| Phage-1647 | Q | — | — | — | — | — | T | — | P | — | — | V | — | — | 0.08 | 1.13 | 0.09 | 1943 |
| Phage-1648 | T | — | S | — | — | — | T | — | T | — | — | V | — | F | 0.06 | 1.13 | 0.06 | 1944 |
| Phage-1649 | T | — | S | — | — | — | H | — | E | — | — | K | — | — | 0.09 | 1.12 | 0.07 | 1945 |
| Phage-1650 | T | — | S | — | — | — | T | — | S | — | — | L | — | F | 0.07 | 1.12 | 0.09 | 1946 |
| Phage-1651 | T | — | — | — | — | — | T | — | S | — | — | S | — | F | 0.06 | 1.10 | 0.07 | 1947 |
| Phage-1652 | T | — | — | — | — | — | N | — | — | — | — | V | — | — | 0.06 | 1.09 | 0.13 | 1948 |
| Phage-1653 | T | — | — | — | — | — | H | — | E | — | — | Q | — | F | 0.06 | 1.08 | 0.06 | 1949 |
| Phage-1654 | T | — | — | — | — | — | T | — | N | — | — | W | — | F | 0.07 | 1.07 | 0.07 | 1950 |
| Phage-1655 | T | — | — | — | — | — | T | W | T | — | — | T | — | M | 0.06 | 1.07 | 0.06 | 1951 |
| Phage-1656 | P | — | — | — | — | — | Q | W | S | — | — | M | — | — | 0.07 | 1.07 | 0.09 | 1952 |
| Phage-1657 | — | — | — | — | — | S | T | W | — | — | — | A | — | — | 0.05 | 1.07 | 0.07 | 1953 |
| Phage-1658 | — | — | — | — | — | F | H | — | E | — | — | Q | — | F | 0.05 | 1.06 | 0.06 | 1954 |
| Phage-1659 | — | — | T | — | — | F | R | I | H | — | — | M | — | F | 0.06 | 1.06 | 0.12 | 1955 |
| Phage-1660 | — | — | — | — | — | — | A | — | — | — | — | W | — | — | 0.06 | 1.05 | 0.07 | 1956 |
| Phage-1661 | M | — | T | — | — | F | — | F | N | — | — | E | — | — | 0.07 | 1.05 | 0.07 | 1957 |
| Phage-1662 | L | — | D | Q | F | F | H | — | N | — | — | A | — | — | 0.06 | 1.04 | 0.05 | 1958 |
| Phage-1663 | — | — | — | — | V | F | — | F | N | — | — | E | — | F | 0.07 | 1.01 | 0.07 | 1959 |
| Phage-1664 | T | — | S | — | — | — | — | — | S | — | — | — | — | M | 0.07 | 1.00 | 0.07 | 1960 |
| Phage-1665 | W | — | S | E | V | F | — | — | P | — | — | A | — | — | 0.06 | 0.99 | 0.06 | 1961 |
| Phage-1666 | — | — | T | — | V | F | — | — | — | L | S | P | — | F | 0.05 | 0.99 | 0.05 | 1962 |
| Phage-1667 | L | — | — | — | V | F | A | — | T | — | — | M | — | F | 0.06 | 0.98 | 0.07 | 1963 |
| Phage-1668 | P | — | — | — | — | — | H | — | V | — | — | V | — | F | 0.07 | 0.97 | 0.06 | 1964 |
| Phage-1669 | T | — | S | — | — | — | T | — | K | — | — | T | — | — | 0.05 | 0.97 | 0.07 | 1965 |
| Phage-1670 | — | — | — | — | — | — | H | W | S | — | — | A | — | — | 0.06 | 0.97 | 0.06 | 1966 |
| Phage-1671 | M | — | — | — | — | — | M | — | N | — | — | L | — | — | 0.07 | 0.95 | 0.09 | 1967 |
| Phage-1672 | — | — | — | — | — | — | — | — | S | — | — | L | — | F | 0.06 | 0.95 | 0.08 | 1968 |
| Phage-1673 | V | — | — | — | — | — | A | — | P | — | — | A | — | — | 0.06 | 0.93 | 0.07 | 1969 |
| Phage-1674 | T | — | S | — | — | — | H | — | P | — | — | M | — | — | 0.07 | 0.93 | 0.06 | 1970 |
| Phage-1675 | T | — | — | — | — | — | T | W | V | — | — | T | — | F | 0.07 | 0.92 | 0.07 | 1971 |
| Phage-1676 | T | — | S | — | — | — | M | — | N | — | — | E | — | F | 0.08 | 0.92 | 0.08 | 1972 |
| Phage-1677 | T | — | S | — | — | — | M | — | S | — | — | L | — | I | 0.06 | 0.91 | 0.08 | 1973 |
| Phage-1678 | Q | — | — | — | — | F | — | — | T | — | — | E | — | Y | 0.06 | 0.90 | 0.11 | 1974 |
| Phage-1679 | W | — | — | — | F | F | H | — | P | — | — | Q | — | F | 0.05 | 0.87 | 0.05 | 1975 |
| Phage-1680 | R | — | K | — | — | V | P | — | K | — | — | M | — | — | 0.06 | 0.87 | 0.06 | 1976 |
| Phage-1681 | T | — | — | — | — | — | T | — | E | — | — | S | — | I | 0.07 | 0.86 | 0.09 | 1977 |
| Phage-1682 | T | — | — | — | — | — | K | — | V | — | — | L | — | F | 0.09 | 0.85 | 0.12 | 1978 |
| Phage-1683 | L | — | E | — | — | F | N | — | D | — | — | R | — | — | 0.06 | 0.85 | 0.07 | 1979 |
| Phage-1684 | T | — | — | — | — | — | — | W | D | — | — | F | — | F | 0.07 | 0.82 | 0.08 | 1980 |
| Phage-1685 | E | — | G | W | E | P | G | Q | H | — | R | V | — | F | 0.06 | 0.82 | 0.06 | 1981 |
| Phage-1686 | — | — | — | — | — | — | T | — | E | — | — | T | — | — | 0.08 | 0.81 | 0.07 | 1982 |
| Phage-1687 | — | — | — | — | — | — | T | — | K | — | — | A | — | F | 0.05 | 0.80 | 0.06 | 1983 |
| Phage-1688 | — | — | S | — | — | — | M | W | L | — | — | E | — | F | 0.05 | 0.80 | 0.06 | 1984 |
| Phage-1689 | W | — | S | — | V | F | — | — | Q | — | — | P | — | F | 0.06 | 0.78 | 0.06 | 1985 |
| Phage-1690 | T | — | S | — | — | — | T | — | K | — | — | M | — | F | 0.06 | 0.77 | 0.07 | 1986 |
| Phage-1691 | T | — | S | — | — | — | T | — | G | — | — | A | — | F | 0.07 | 0.76 | 0.07 | 1987 |
| Phage-1692 | I | — | — | — | — | — | G | W | P | — | — | W | — | — | 0.08 | 0.75 | 0.10 | 1988 |
| Phage-1693 | E | — | S | — | — | — | M | W | G | — | — | A | — | F | 0.06 | 0.73 | 0.09 | 1989 |
| Phage-1694 | T | — | — | — | — | — | T | — | D | — | — | S | — | — | 0.06 | 0.71 | 0.08 | 1990 |
| Phage-1695 | T | — | — | — | — | — | V | W | Q | — | — | T | — | F | 0.06 | 0.71 | 0.06 | 1991 |
| Phage-1696 | P | — | S | — | — | — | T | — | — | — | — | — | — | — | 0.06 | 0.71 | 0.09 | 1992 |
| Phage-1697 | T | — | — | — | — | — | Q | L | E | — | — | W | — | F | 0.09 | 0.70 | 0.07 | 1993 |
| Phage-1698 | G | — | — | — | — | — | E | — | N | — | — | T | — | — | 0.05 | 0.69 | 0.64 | 1994 |
| Phage-1699 | T | — | — | — | — | — | A | — | T | — | — | H | — | F | 0.09 | 0.69 | 0.11 | 1995 |
| Phage-1700 | T | — | S | — | — | — | Q | W | E | — | — | A | — | — | 0.06 | 0.67 | 0.08 | 1996 |
| Phage-1701 | W | — | S | — | V | F | — | — | Q | — | — | V | — | — | 0.07 | 0.61 | 0.06 | 1997 |
| Phage-1702 | — | — | S | — | — | — | — | — | — | — | — | M | — | I | 0.06 | 0.59 | 0.06 | 1998 |
| Phage-1703 | D | — | W | W | D | S | P | — | H | — | R | V | — | — | 0.06 | 0.57 | 0.07 | 1999 |
| Phage-1704 | — | — | — | — | — | — | G | — | G | — | — | Q | — | — | 0.07 | 0.55 | 0.13 | 2000 |
| Phage-1705 | — | — | G | G | — | F | H | — | S | — | — | L | — | F | 0.07 | 0.55 | 0.15 | 2001 |
| Phage-1706 | G | — | — | — | — | F | H | — | T | L | S | T | — | — | 0.05 | 0.42 | 0.05 | 2002 |

In some embodiments, P₁ comprises an amino acid sequence according to: J₁-J₂-W-C-J₃-J₄-J₅-J₆-J₇-J₈-J₉-C-J₁₀-J₁₁, wherein J₁ is selected from A, R, N, D, Q, E, G, H, I, L, K, F, P, S, T, W, Y, and V; J₂ is selected from D, P, and Y; J₃ is selected from P and Q; J₄ is selected from R, L, and I; J₄ is selected from R. L, and I: J₅ is selected from D, H, Y, and I; J₅ is selected from D, H, Y, and I; J₆ is selected from L, S, and R; J₇ is selected from W and G: J₅ is selected from V, A, D, H, L, and N; J₉ is selected from H, L, E, and D: J₁₀ is selected from F, Y, and L; and J₁₁ is selected from A, N, H, and T. In some embodiments, J₁ is selected from A, R, N, D, Q, E, G, H, K, F, P, S, T, W, and Y. In some embodiments, J₁ is selected from R, Q, E, H, I, L, K, F, T, W, Y, and V; J₂ is D; J₃ is P: J is P: J₄ is R; J₅ is D; J is L; J₇ is W; J₈ is V; J₉ is H: J₁₀ is F; and J₁₁ is A. In some embodiments. J₁ is selected from R, Q, E, L, K, T, and W: J₂ is D: J₃ is P: J₄ is R; J₅ is D; J₆ is L; J₇ is W; J₈ is V; J₉ is H; J₁₀ is F; and J₁₁ is A. In some embodiments, J₁ is L or I; J₂ is D or Y; J₃ is P: J₄ is R, L₁ or; J₅ is H, Y, or D; J₆ is L or R; J₇ is W; J₈ is A, D, H, or N; J₉ is L, E, or D; J₁₀ is F or Y; and J₁₁ is N or H. In some embodiments, J₁ is L or I; J₂ is D or Y: J₃ is P: J₄ is R or I; J₅ is H. Y. or D; J₆ is L or R: J₇ is W; J₈ is A, D, or N; J is L, E, or D; J₁1 is For Y; and J₁₁ is N.

In some embodiments, P₁ comprises an amino acid sequence according to: Z₁-C-Z₂-Z₃-Z₄-Z₅-Z₆-Z₇-Z₈-Z₉-Z₁₀-Z₁₁-C-Z₁₂, wherein Z, is selected from Y, H, and A; Z₂ is selected from D, E, A, Q, S, V, M, H, L, F, T, W, and N: Z₃ is selected from G, E, S, and A; Z₄ is selected from H, L, W, and A; Z₈ is selected from L, S, A. T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, Q, and A: Z₁₀ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₁₂ is selected from L, M, I, F, V, Y, Q, D, T, and A. In some embodiments, Z₁ is selected from Y and H: Z₂ is selected from D, E, A, Q, S, V, M, H, L, F, T, W, and N; Z₃ is selected from G, E, and S: Z₄ is selected from H, L, and W: Z₈ is selected from L, S, A, T, I, V, M, R, E, D, F, H, K, Q, N, Y, W, and G: Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N: Z₈ is selected from Y, H, F, W, N, and Q; Z₉ is selected from M, I, L, R, V, K, F, Q, Y, and W: Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Z₁ is selected from Y and H; Z₂ is selected from D, E, A, Q, S, V, M, H, L, F, T, W, and N: Z₃ is selected from G, E, and S; Z₄ is selected from H, L, and W; Z₈ is selected from L, S, A, T, I, V, M, R, E, D, F, H, K, Q, N, Y, W, and G; Z₆ is selected from P, A, E, Q, S, L, W, G, V, D, K, I, M, R T, F, H, N, and Y; Z₇ is selected from E, Q, I, M, V, A, L, R, W, P, D, T, K, S, G, H, and N; Z₈ is selected from Y, H, F, W, N, and Q; Z₉ is selected from M, I, L, R, V, K, F, Q, Y, and W: Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Z, is Y; Z₂ is selected from D, E, A, and Q; Z₃ is G; Z₄ is H; Z₈ is selected from L, S, A, T, I, V, and M; Z₆ is selected from P, A, E, Q, S, L, W, G, V, and D: Z, is selected from E, Q, I, M, V, A, and L; Z₈ is selected from Y and H; Z₉ is selected from M, I, and L: Z₁₀ is selected from A, Q, S, W, E, L, G, D, T, M, and N: Z₁₁ is K; and Z₁₂ is selected from L and M. In some embodiments, Z₁ is Y; Z₂ is selected from D, E, and A: Z₃ is G; Z₄ is H; Z₄ is H; Z₈ is selected from L, S, A, and T; Z₆ is selected from P, A, E, Q, S, L, and W: Z, is selected from E, Q, I, and M; Z₈ is Y; Z₉ is selected from M, I, and L; Z₁₀ is selected from A. Q, S, W, E, L, G, and D: Z₁₁ is K; and Z₁₂ is L. In some embodiments, Z, is Y: Z₂ is selected from D and E; Z₃ is G; Z₄ is H; Z₈ is selected from L, S, and A; Z₆ is selected from P, A, E, Q, S, and L; Z, is selected from E, Q, and I; Z₈ is Y; Z₉ is selected from M, I, and L: Z₁₀ is selected from A, Q, S, W, E, L, G, and D; Z₁₁ is K; and Z₂ is L.

In some embodiments. P₁ comprises the amino acid sequence of SEQ ID NO: 784. In some embodiments, P, consists of the amino acid sequence of SEQ ID NO: 784. In some embodiments, P, comprises the amino acid sequence of SEQ ID NO: 744. In some embodiments, P, consists of the amino acid sequence of SEQ ID NO: 744. In some embodiments, P₁ comprises an amino acid sequence according to any one of SEQ ID NOs: 774-793. In some embodiments, P₁ comprises an amino acid sequence according to any one of SEQ ID NOs: 764-773.

In some embodiments, P₁ comprises an amino acid sequence according to: U₁-C-U₂-U₃-U₄-U₈-U₆-U₇-U₈-U₉-U₁₀-U₁₁-C-U₁₂, wherein: U₁ is selected from T, S, L, M, W, Q, P, R, G, N, I, Y, D, E, V, H, and A; U₂ is selected from A, S, T, K, E, R, G, N, W, and D; U₃ is selected from A, E, P, D, G, W, Q, V, and F: U₄ is selected from G, V, F, D, E, R, and A; U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, I, and A; U₈ is selected from P, S, T, A, E, K, Q, N, V, G, H, L, D, Y, I, IM, and R; U₉ is selected from W, L, and A: U₁₀ is selected from P, S, R, L, E, and A; U₁₁ is selected from L, A, T, M, V, W, Y, E, Q, I, F, S, P, K, R, D, H, N, and G; and U₂ is selected from L, F, V, M, I, Y, E, W, and A. In some embodiments, U₁ is selected from T, S, L, M, W, Q, P, R, G, N, I, Y, D, E, V, and H; U₂ is selected from A, S, T, K, E, R, G, N, W, and D: U₃ is selected from A, E, P, D, G, W, Q, V, and F: U₄ is selected from G, V, F, D, E, and R: U₅ is selected from A, F, V, S, L, P, and M; U₆ is selected from T, S, H, M, A, N, P, G, Q, D, Y, V, L, I, E, K, F, and R; U₇ is selected from Y, W, F, L, N, T, Q, and I: U₈ is selected from P, S, T, A, E, K, Q, N, V, G, H, L, D, Y, I, M, and R: U₉ is selected from W, L, and A; U₁₀ is selected from P, S, R, L, and E; U₁₁ is selected from L, A, T, M, V, W, Y, E, Q, I, F, S, P, K, R, D, H, N, and G; and U₂ is selected from L, F, V, M, I, Y, E, and W. In some embodiments, U₁ is selected from T, S, L, M, and W; U₂ is selected from A, S, and T; U₃ is selected from A and E; U₄ is selected from G, V, and F; U₈ is selected from A, F, and V; U₆ is selected from T, S, H, M, A, and N; U₇ is selected from Y, W, and F: U₈ is selected from P, S, T, A, E, K, Q, N, and V; U₉ is selected from W and L; U₁₀ is selected from P and S; U₁₁ is selected from L, A, T, M, V, W, Y, E, Q, I, F, and S; and U₁₂ is selected from L, F, V, and M. In some embodiments, U, is selected from T and S; U₂ is selected from A and S; U₃ is A; U₄ is selected from G and V; U₅ is selected from A and F; U₆ is selected from T, S, and H; U₇ is selected from Y and W; U₈ is selected from P, S, T, and A; U₉ is W; U₁₀ is P; U₁₁ is selected from L, A, T, M, V, W, and Y; and U₁₂ is selected from L and F.

In some embodiments, P₁ comprises the amino acid sequence of SEQ ID NO: 749. In some embodiments, P₁ comprises the amino acid sequence of SEQ ID NO: 749. In some embodiments, P, comprises an amino acid sequence according to any one of SEQ ID NOs: 756-763. In some embodiments, P₁ comprises an amino acid sequence according to any one of SEQ ID NOs: 794-813. In some embodiments, P₁ comprises an amino acid sequence according to any one of SEQ ID NOs: 732-2002.

In some embodiments, P₂ comprises the amino acid sequence of any one of SEQ ID NOs: 2003-4329. In some embodiments, P₂ comprises the amino acid sequence of SEQ ID NO: 2025. In some embodiments, P₂ consists of the amino acid sequence of SEQ ID NO: 2025. In some embodiments. P₂ comprises the amino acid sequence according to any one of SEQ ID NOs: 2087-2094. In some embodiments, P₂ comprises the amino acid sequence of SEQ ID NO: 2026. In some embodiments, P₂ consists of the amino acid sequence of SEQ ID NO: 2026. In some embodiments, P₂ comprises the amino acid sequence according to any one of SEQ ID NOs: 2097-2106. In some embodiments, P₂ comprises the amino acid sequence according to any one of SEQ ID NOs: 2107-2133. In some embodiments. P₂ comprises the amino acid sequence of SEQ ID NO: 2120. In some embodiments. P₂ consists of the amino acid sequence of SEQ ID NO: 2120.

In some embodiments, P₂ comprises an amino acid sequence according to B₁-B₂-B₃-B₄-C-B₅-P-B₆-W-B₇-C-B-B₉-B₁₀ (SEQ ID NO: 4470), wherein: B, is selected from V, E, L, D, I, G, M, S, P, T, A, F, W, Y, Q, H, N, K, and R; B₂ is selected from E, V, D, T, S, L, G, P, A, M, I, Q, H, F, Y, N, W, R, and K; B₃ is selected from K, P, R, I, N, H, V, M, A, L, Q, T, S, G, F, Y, E, W, and D; B₄ is selected from W, L, M, R, V, Y, A, K, I, S, Q, F, H, E, T, N, G, and D: B₅ is selected from I, V, T, K, R, E, S, Q M, L, F, A, N, and H; B₆ is selected from A, E, S, P, Q, T, L, D, M, V, R, K, N, I, H, W, Y, F, and G; B₇ is selected from M, L, I, A, V, F, G, and K; B₈ is selected from E, S, T, A, V, D, Q, L, N, I, M, Y, H, F, W, G, K, R, and P; B₉ is selected from G, V, A, S, W, E, D, M, T, L, F, H, Q, N, R, I, Y, P, and K; and B₁₀ is selected from F, L, M, S, I, V, D, Q, T, A, N, Y, W, E, R, H, P, G, and K.

TABLE 9
PSMA Peptide Mask Sequences (P2)

| | Amino Acid | |
|---|---|---|
| Construct | Sequence | SEQ ID |
| Description | (N to C) | NO: |
| Peptide-157 | DCFYAADIHWNYCN | 2003 |
| Peptide-158 | YECHQGLFHRWCVN | 2004 |
| Peptide-159 | SCFSFARLEVVPCD | 2005 |
| Peptide-160 | TCLRFADLAIIHCD | 2006 |
| Peptide-161 | NCWWHDFRAGHVVC | 2007 |
| Peptide-162 | IRCVGEGHDLVCIF | 2008 |
| Peptide-163 | SIPCIPRDWLCPIK | 2009 |
| Peptide-164 | ICQWIGATNAWVCY | 2010 |
| Peptide-165 | HLFCWEGSDRCAWV | 2011 |
| Peptide-166 | PLCGTEEVWWECRL | 2012 |
| Peptide-167 | NAHCWLWVPAHCRV | 2013 |
| Peptide-168 | FGHCAGSPVFCAYW | 2014 |
| Peptide-169 | NCFFAADIHWNFCT | 2015 |
| Peptide-170 | FCQWIEATNAWLCY | 2016 |
| Peptide-171 | FCGWMDGDVFWFCG | 2017 |
| Peptide-172 | FCNWVDGDVWVVCK | 2018 |
| Peptide-173 | EGCLTLVDGRAWCW | 2019 |
| Peptide-174 | MEYCSFHRFTCVLW | 2020 |
| Peptide-175 | HCKFHDYHSWYMCY | 2021 |
| Peptide-176 | ACHSLVLKHHLQKC | 2022 |
| Peptide-177 | SCGWLRGEEFGWWC | 2023 |
| Peptide-178 | PDCVYHSFWGYSCR | 2024 |
| Peptide-179 | LMKWCTPRWMCAAE | 2025 |
| Peptide-180 | IAVLCQPNWMCAGF | 2026 |
| Peptide-181 | PELRCVPFWMCSFN | 2027 |
| Peptide-182 | SIPCIPRDWLCPIK | 2028 |
| Peptide-183 | MEYCSFHRFTCVLW | 2029 |
| Peptide-184 | IRCVGEGHDLVCIF | 2030 |
| Peptide-185 | YECHQGLFHRWCVN | 2031 |
| Peptide-186 | FVCWTFGEHIKVCR | 2032 |
| Peptide-187 | ILCGLYEALVSECG | 2033 |
| Peptide-188 | IECAYMTDQRFWCW | 2034 |
| Peptide-189 | HCLGPALGPVDGDC | 2035 |
| Peptide-190 | SCGWLRGEEFGWWC | 2036 |
| Peptide-191 | FVPCIPSHWLCPIW | 2037 |
| Peptide-192 | HCLWHESSGRVLCF | 2038 |
| Peptide-193 | MEYCSFHRFTCVLW | 2039 |
| Peptide-194 | SKLLCVPPWMCYHV | 2040 |
| Peptide-195 | QLPCHYWGEWCPQA | 2041 |
| Peptide-196 | FWECYSSFCRFVEG | 2042 |
| Peptide-197 | PLCGTEEVWWECRL | 2043 |
| Peptide-198 | LTRCVPDWMCYRAV | 2044 |
| Peptide-199 | YCWVDADMWLIFRC | 2045 |
| Peptide-200 | ILPCHWYTSDCPQH | 2046 |
| Peptide-201 | WCWVQDDRRHVVCG | 2047 |
| Peptide-202 | MCQWDELNYWMDCD | 2048 |
| Peptide-203 | QCVWDNFNYWMDCD | 2049 |
| Peptide-204 | QCTWNEKTWWMECE | 2050 |
| Peptide-205 | MCQWDKINYWMDCD | 2051 |
| Peptide-206 | QCVWDLTNYWMNCD | 2052 |
| Peptide-207 | DCHWNKATWWMDCE | 2053 |
| Peptide-208 | MCFVTGWGVPDDCN | 2054 |
| Peptide-209 | QCVWDRGTWWMACD | 2055 |
| Peptide-210 | LCQWYPKFHWMDCD | 2056 |
| Peptide-211 | HCEFHDYQSCDVLY | 2057 |
| Peptide-212 | HCKFHDYHSWYVCY | 2058 |
| Peptide-213 | LCEWWTSHVDAWCA | 2059 |
| Peptide-214 | WEKLCTPAWMCVGF | 2060 |
| Peptide-215 | LEKLCIPAWMCEWL | 2061 |
| Peptide-216 | VLKWCRPAWMCEVL | 2062 |
| Peptide-217 | VGKLCVPEWMCVGL | 2063 |
| Peptide-218 | FVPLCKPAWMCSFV | 2064 |
| Peptide-219 | HVKLCKPAWMCQGL | 2065 |
| Peptide-220 | FEKLCVPKWMCEGI | 2066 |
| Peptide-221 | IDKLCIPFWMCENL | 2067 |
| Peptide-222 | FEKWCTPPWMCSLF | 2068 |
| Peptide-223 | DLRLCKPAWMCYFI | 2069 |
| Peptide-224 | EGIRCTPIWMCEGF | 2070 |
| Peptide-225 | IPKSCIPSWMCTGF | 2071 |
| Peptide-226 | LTKLCIPDWMCSEF | 2072 |
| Peptide-227 | LDNWCKPSWMCTGF | 2073 |
| Peptide-228 | EFKWCIPSWMCHET | 2074 |
| Peptide-229 | VVKACIPTWMCQFV | 2075 |
| Peptide-230 | VTKWCIPPWMCEVT | 2076 |
| Peptide-231 | DLRLCKPAWMCDFI | 2077 |
| Peptide-232 | LDKWCVPKWMCSGL | 2078 |
| Peptide-233 | GEKWCIPDWMCSGF | 2079 |
| Peptide-234 | LEKTCIPTWMCVGF | 2080 |
| Peptide-235 | VEKLCTPIWMCLGF | 2081 |
| Peptide-236 | DVKLCKPEWMCYGL | 2082 |
| Peptide-237 | ISKWCIPEWMCTSP | 2083 |
| Peptide-238 | LEKLCKPAWMCVNF | 2084 |
| Peptide-239 | IDIRCTPIWMCVGF | 2085 |
| Peptide-240 | EPKWCVPRGLCNYD | 2086 |
| Peptide-241 | AMKWCTPRWMCAAE | 2087 |
| Peptide-242 | LAKWCTPRWMCAAE | 2088 |
| Peptide-243 | LMAWCTPRWMCAAE | 2089 |
| Peptide-244 | LMKACTPRWMCAAE | 2090 |
| Peptide-245 | LMKWCAPRWMCAAE | 2091 |
| Peptide-246 | LMKWCTARWMCAAE | 2092 |
| Peptide-247 | LMKWCTPAWMCAAE | 2093 |
| Peptide-248 | LMKWCTPRAMCAAE | 2094 |
| Peptide-249 | LMKWCTPRWACAAE | 2095 |
| Peptide-250 | LMKWCTPRWMC | 2096 |
| Peptide-251 | AAVLCQPNWMCAGF | 2097 |
| Peptide-252 | IAALCQPNWMCAGF | 2098 |
| Peptide-253 | IAVACQPNWMCAGF | 2099 |
| Peptide-254 | IAVLCAPNWMCAGF | 2100 |
| Peptide-255 | IAVLCQANWMCAGF | 2101 |
| Peptide-256 | IAVLCQPAWMCAGF | 2102 |
| Peptide-257 | IAVLCQPNAMCAGF | 2103 |
| Peptide-258 | IAVLCQPNWACAGF | 2104 |
| Peptide-259 | IAVLCQPNWMCAAF | 2105 |
| Peptide-260 | IAVLCQPNWMCAGA | 2106 |
| Peptide-261 | YFVQCRPAWMCEGF | 2107 |
| Peptide-262 | IVPLCKPAWMCKLF | 2108 |
| Peptide-263 | AVKACIPEWMCIGF | 2109 |
| Peptide-264 | VQIQCRPAWMCLGF | 2110 |
| Peptide-265 | AEVKCIPQWMCQGF | 2111 |
| Peptide-266 | TAHHCKPAWMCTGF | 2112 |
| Peptide-267 | FTNYCVPSWMCTGF | 2113 |
| Peptide-268 | VEKVCKPVWMCVGF | 2114 |
| Peptide-269 | EEPLCRPVWMCEGF | 2115 |
| Peptide-270 | ESRLCVPAWMCAGF | 2116 |
| Peptide-271 | EDILCKPKWMCEGF | 2117 |
| Peptide-272 | LHVYCVPEWMCAGF | 2118 |
| Peptide-273 | LEPLCVPRWMCVGF | 2119 |
| Peptide-274 | ITPVCKPLWMCSGL | 2120 |
| Peptide-275 | VEVKCFPLWFCVGL | 2121 |
| Peptide-276 | ITPICKPAWICSGF | 2122 |
| Peptide-277 | VVIKCKPEWMCSGF | 2123 |
| Peptide-278 | PEHLCLPRWMCEGF | 2124 |
| Peptide-279 | YERACIPNWICEGF | 2125 |
| Peptide-280 | TTHVCIPSWMCSGF | 2126 |
| Peptide-281 | VNHWCVPTWMCAGF | 2127 |
| Peptide-282 | IVVKCVPSWMCEGF | 2128 |
| Peptide-283 | TVAYCKPAWMCQGF | 2129 |
| Peptide-284 | TEVRCTPPWMCLGF | 2130 |
| Peptide-285 | VERLCTPAWMCHGF | 2131 |
| Peptide-286 | VLKYCIPDWMCAGI | 2132 |
| Peptide-287 | PERLCTPPWMCLGF | 2133 |

TABLE 10
Phage Panning Results of Peptide-180 Library Sequences.

| | | | | | | | | | | | | | | | Phage binding ELISA | | | |
|---|---|---|---|---|---|---|---|---|---|---|---|---|---|---|---|---|---|---|
| | | | | | | | | | | | | | | | | Anti- | Anti- | |
| | | | | | | | | | | | | | | | | PSMA | PSMA | |
| | | | | | | | | | | | | | | | | Fab | Fab | |
| | | | | | | | | | | | | | | | | (SEQ ID | signal | |
| | | | | | | | | | | | | | | | | NOs: 19, | in the | SEQ |
| | Amino acid position sequence | | | | | | | | | | | | | | Background | 20) | presence | ID |
| Phage ID | 1 | 2 | 3 | 4 | 5 | 6 | 7 | 8 | 9 | 10 | 11 | 12 | 13 | 14 | signal | signal | of PSMA | NO: |
| Phage- | I | A | V | L | C | Q | P | N | W | M | C | A | G | F | 0.078 | 0.997 | 0.077 | 2026 |
| 1708 | | | | | | | | | | | | | | | | | | |
| Phage- | L | M | K | W | — | T | — | R | — | — | — | — | A | E | 0.222 | 1.043 | 0.232 | 2025 |
| 1707 | | | | | | | | | | | | | | | | | | |
| Phage- | P | E | L | R | — | V | — | F | — | — | — | S | F | N | 0.118 | 0.622 | 0.064 | 2027 |
| 1709 | | | | | | | | | | | | | | | | | | |
| Phage- | S | I | P | C | I | P | R | D | — | L | — | P | I | K | 0.068 | 1.889 | 0.096 | 2028 |
| 1710 | | | | | | | | | | | | | | | | | | |
| Phage- | M | E | Y | C | S | F | H | R | F | T | — | V | L | W | 0.101 | 1.878 | 0.117 | 2029 |
| 1711 | | | | | | | | | | | | | | | | | | |
| Phage- | — | R | C | V | G | E | G | H | D | L | V | C | I | — | 0.073 | 2.364 | 0.072 | 2030 |
| 1712 | | | | | | | | | | | | | | | | | | |
| Phage- | Y | E | C | H | Q | G | L | F | H | R | W | C | V | N | 0.099 | 0.759 | 0.074 | 2031 |
| 1713 | | | | | | | | | | | | | | | | | | |
| Phage- | F | V | C | W | T | F | G | E | H | I | K | V | C | R | 0.210 | 2.512 | 0.205 | 2032 |
| 1714 | | | | | | | | | | | | | | | | | | |
| Phage- | — | L | C | G | L | Y | E | A | L | V | S | E | C | G | 0.067 | 2.491 | 0.063 | 2033 |
| 1715 | | | | | | | | | | | | | | | | | | |
| Phage- | — | E | C | A | Y | M | T | D | Q | R | F | W | C | W | 0.060 | 1.934 | 0.066 | 2034 |
| 1716 | | | | | | | | | | | | | | | | | | |
| Phage- | H | C | L | G | P | A | L | G | P | V | D | G | D | C | 0.063 | 2.329 | 0.076 | 2035 |
| 1717 | | | | | | | | | | | | | | | | | | |
| Phage- | S | C | G | W | L | R | G | E | E | F | G | W | W | C | 0.077 | 0.648 | 0.071 | 2036 |
| 1718 | | | | | | | | | | | | | | | | | | |
| Phage- | D | C | F | Y | A | A | D | I | H | W | N | Y | C | N | 0.0514 | 1.8787 | 1.1521 | 2003 |
| 1719 | | | | | | | | | | | | | | | | | | |
| Phage- | Y | E | C | H | Q | G | L | F | H | R | W | C | V | N | 0.0684 | 0.6878 | 0.073 | 2004 |
| 1720 | | | | | | | | | | | | | | | | | | |
| Phage- | S | C | F | S | F | A | R | L | E | V | V | P | C | D | 0.0498 | 1.2593 | 0.0555 | 2005 |
| 1721 | | | | | | | | | | | | | | | | | | |
| Phage- | T | C | L | R | F | A | D | L | A | I | I | H | C | D | 0.0576 | 1.8193 | 0.0697 | 2006 |
| 1722 | | | | | | | | | | | | | | | | | | |
| Phage- | N | C | W | W | H | D | F | R | A | G | H | V | V | C | 0.2274 | 2.0504 | 0.0972 | 2007 |
| 1723 | | | | | | | | | | | | | | | | | | |
| Phage- | — | R | C | V | G | E | G | H | D | L | V | C | I | — | 0.0689 | 2.125 | 0.081 | 2008 |
| 1724 | | | | | | | | | | | | | | | | | | |
| Phage- | S | I | P | C | I | P | R | D | — | L | — | P | I | K | 0.0543 | 1.7989 | 0.239 | 2009 |
| 1725 | | | | | | | | | | | | | | | | | | |
| Phage- | — | C | Q | W | I | G | A | T | N | A | W | V | C | Y | 0.062 | 1.8247 | 0.3259 | 2010 |
| 1726 | | | | | | | | | | | | | | | | | | |
| Phage- | H | L | F | C | W | E | G | S | D | R | — | — | W | V | 0.0983 | 1.4028 | 0.0753 | 2011 |
| 1727 | | | | | | | | | | | | | | | | | | |
| Phage- | P | L | C | G | T | E | E | V | — | W | E | C | R | L | 0.0697 | 1.5671 | 0.0694 | 2012 |
| 1728 | | | | | | | | | | | | | | | | | | |
| Phage- | N | — | H | C | W | L | W | V | P | A | H | C | R | V | 0.0611 | 1.3543 | 0.0672 | 2013 |
| 1729 | | | | | | | | | | | | | | | | | | |
| Phage- | F | G | H | C | A | G | S | P | V | F | — | — | Y | W | 0.0525 | 1.0636 | 0.054 | 2014 |
| 1730 | | | | | | | | | | | | | | | | | | |
| Phage- | N | C | F | F | A | A | D | I | H | W | N | F | C | T | 0.0512 | 1.4806 | 0.1797 | 2015 |
| 1731 | | | | | | | | | | | | | | | | | | |
| Phage- | F | C | Q | W | I | E | A | T | N | A | W | L | C | Y | 0.0553 | 1.065 | 0.1188 | 2016 |
| 1732 | | | | | | | | | | | | | | | | | | |
| Phage- | F | C | G | W | M | D | G | D | V | F | W | F | C | G | 0.0605 | 0.428 | 0.1447 | 2017 |
| 1733 | | | | | | | | | | | | | | | | | | |
| Phage- | F | C | N | W | V | D | G | D | V | W | V | V | C | K | 0.0496 | 0.5102 | 0.0607 | 2018 |
| 1734 | | | | | | | | | | | | | | | | | | |
| Phage- | E | G | C | — | T | L | V | D | G | R | A | W | C | W | 0.0509 | 1.0481 | 0.0521 | 2019 |
| 1735 | | | | | | | | | | | | | | | | | | |
| Phage- | M | E | Y | C | S | F | H | R | F | T | — | V | L | W | 0.0992 | 1.7441 | 0.1199 | 2020 |
| 1736 | | | | | | | | | | | | | | | | | | |
| Phage- | H | C | K | F | H | D | Y | H | S | W | Y | M | C | Y | 0.0581 | 1.584 | 0.1175 | 2021 |
| 1737 | | | | | | | | | | | | | | | | | | |
| Phage- | A | C | H | S | L | V | L | K | H | H | L | q | K | C | 0.0546 | 1.1802 | 0.0624 | 2022 |
| 1738 | | | | | | | | | | | | | | | | | | |
| Phage- | S | C | G | W | L | R | G | E | E | F | G | W | W | C | 0.0606 | 0.8909 | 0.0625 | 2023 |
| 1739 | | | | | | | | | | | | | | | | | | |
| Phage- | P | D | C | V | Y | H | S | F | — | G | Y | S | C | R | 0.054 | 0.5833 | 0.0641 | 2024 |
| 1740 | | | | | | | | | | | | | | | | | | |
| Phage- | F | V | P | C | I | P | S | H | — | L | — | P | I | W | 0.0847 | 1.0458 | 0.2917 | 2037 |
| 1741 | | | | | | | | | | | | | | | | | | |
| Phage- | H | C | L | W | H | E | S | S | G | R | V | L | C | — | 0.0521 | 2.1539 | 0.0678 | 2038 |
| 1742 | | | | | | | | | | | | | | | | | | |
| Phage- | M | E | Y | C | S | F | H | R | F | T | — | V | L | W | 0.1145 | 1.9307 | 0.116 | 2039 |
| 1743 | | | | | | | | | | | | | | | | | | |
| Phage- | S | K | L | — | — | V | — | P | — | — | — | Y | H | V | 0.0735 | 0.8057 | 0.0794 | 2040 |
| 1744 | | | | | | | | | | | | | | | | | | |
| Phage- | Q | L | P | C | H | Y | W | G | E | W | — | P | Q | A | 0.0498 | 1.386 | 0.0537 | 2041 |
| 1745 | | | | | | | | | | | | | | | | | | |
| Phage- | F | W | E | C | Y | S | S | F | C | R | F | V | E | G | 0.0919 | 1.4607 | 0.1391 | 2042 |
| 1746 | | | | | | | | | | | | | | | | | | |
| Phage- | P | L | C | G | T | E | E | V | — | W | E | C | R | L | 0.0598 | 1.7838 | 0.0826 | 2043 |
| 1747 | | | | | | | | | | | | | | | | | | |
| Phage- | L | T | R | C | V | P | D | W | M | C | Y | R | A | V | 0.0778 | 1.1747 | 0.0708 | 2044 |
| 1748 | | | | | | | | | | | | | | | | | | |
| Phage- | Y | C | W | V | D | A | D | M | — | L | I | F | R | C | 0.0566 | 1.013 | 0.2749 | 2045 |
| 1749 | | | | | | | | | | | | | | | | | | |
| Phage- | — | L | P | C | H | W | Y | T | S | D | — | P | Q | H | 0.0869 | 1.631 | 0.1169 | 2046 |
| 1750 | | | | | | | | | | | | | | | | | | |
| Phage- | W | C | W | V | Q | D | D | R | R | H | V | V | C | G | 0.0537 | 1.1859 | 0.0549 | 2047 |
| 1751 | | | | | | | | | | | | | | | | | | |
| Phage- | M | C | Q | W | D | E | L | — | Y | W | M | D | C | D | 0.0572 | 1.6492 | 0.0565 | 2048 |
| 1752 | | | | | | | | | | | | | | | | | | |
| Phage- | Q | C | — | W | D | N | F | — | Y | W | M | D | C | D | 0.0585 | 0.7718 | 0.0667 | 2049 |
| 1753 | | | | | | | | | | | | | | | | | | |
| Phage- | Q | C | T | W | N | E | K | T | — | W | M | E | C | E | 0.0483 | 1.3501 | 0.0568 | 2050 |
| 1754 | | | | | | | | | | | | | | | | | | |
| Phage- | M | C | Q | W | D | K | I | — | Y | W | M | D | C | D | 0.0699 | 1.8117 | 0.049 | 2051 |
| 1755 | | | | | | | | | | | | | | | | | | |
| Phage- | Q | C | — | W | D | L | T | — | Y | W | M | N | C | D | 0.0478 | 1.3041 | 0.057 | 2052 |
| 1756 | | | | | | | | | | | | | | | | | | |
| Phage- | D | C | H | W | N | K | A | T | — | W | M | D | C | E | 0.0545 | 1.1767 | 0.0828 | 2053 |
| 1757 | | | | | | | | | | | | | | | | | | |
| Phage- | M | C | F | V | T | G | W | G | V | P | D | D | C | N | 0.0564 | 0.6382 | 0.0694 | 2054 |
| 1758 | | | | | | | | | | | | | | | | | | |
| Phage- | Q | C | — | W | D | R | G | T | — | W | M | — | C | D | 0.0525 | 0.4143 | 0.052 | 2055 |
| 1759 | | | | | | | | | | | | | | | | | | |
| Phage- | L | C | Q | W | Y | P | K | F | H | W | M | D | C | D | 0.0575 | 0.6113 | 0.0695 | 2056 |
| 1760 | | | | | | | | | | | | | | | | | | |
| Phage- | H | C | E | F | H | D | Y | Q | S | C | D | V | L | Y | 0.0538 | 1.8922 | 0.1259 | 2057 |
| 1761 | | | | | | | | | | | | | | | | | | |
| Phage- | H | C | K | F | H | D | Y | H | S | W | Y | V | C | Y | 0.054 | 2.3079 | 0.0994 | 2058 |
| 1762 | | | | | | | | | | | | | | | | | | |
| Phage- | L | C | E | W | W | T | S | H | V | D | A | W | C | A | 0.0508 | 0.5751 | 0.1839 | 2059 |
| 1763 | | | | | | | | | | | | | | | | | | |
| Phage- | W | E | K | — | — | T | — | A | — | — | — | V | — | — | 0.0628 | 2.4378 | 0.1052 | 2060 |
| 1764 | | | | | | | | | | | | | | | | | | |
| Phage- | L | E | K | — | — | I | — | A | — | — | — | E | W | L | 0.0866 | 2.4002 | 0.1842 | 2061 |
| 1765 | | | | | | | | | | | | | | | | | | |
| Phage- | V | L | K | W | — | R | — | A | — | — | — | E | V | L | 0.1368 | 2.3796 | 0.2186 | 2062 |
| 1766 | | | | | | | | | | | | | | | | | | |
| Phage- | V | G | K | — | — | V | — | E | — | — | — | V | — | L | 0.0567 | 2.2458 | 0.063 | 2063 |
| 1767 | | | | | | | | | | | | | | | | | | |
| Phage- | F | V | P | — | — | K | — | A | — | — | — | S | F | V | 0.0722 | 2.2019 | 0.1114 | 2064 |
| 1768 | | | | | | | | | | | | | | | | | | |
| Phage- | H | V | K | — | — | K | — | A | — | — | — | Q | — | L | 0.0793 | 2.178 | 0.0831 | 2065 |
| 1769 | | | | | | | | | | | | | | | | | | |
| Phage- | F | E | K | — | — | V | — | K | — | — | — | E | — | I | 0.0594 | 2.1734 | 0.0601 | 2066 |
| 1770 | | | | | | | | | | | | | | | | | | |
| Phage- | — | D | K | — | — | I | — | F | — | — | — | E | N | L | 0.0771 | 2.1578 | 0.0699 | 2067 |
| 1771 | | | | | | | | | | | | | | | | | | |
| Phage- | F | E | K | W | — | T | — | P | — | — | — | S | L | — | 0.179 | 2.1128 | 0.2025 | 2068 |
| 1772 | | | | | | | | | | | | | | | | | | |
| Phage- | D | L | R | — | — | K | — | A | — | — | — | Y | F | I | 0.0514 | 2.1122 | 0.0751 | 2069 |
| 1773 | | | | | | | | | | | | | | | | | | |
| Phage- | E | G | I | R | — | T | — | I | — | — | — | E | — | — | 0.0589 | 2.0886 | 0.0778 | 2070 |
| 1774 | | | | | | | | | | | | | | | | | | |
| Phage- | — | P | K | S | — | I | — | S | — | — | — | T | — | — | 0.061 | 2.0848 | 0.1006 | 2071 |
| 1775 | | | | | | | | | | | | | | | | | | |
| Phage- | L | T | K | — | — | I | — | D | — | — | — | S | E | — | 0.0578 | 2.0675 | 0.0876 | 2072 |
| 1776 | | | | | | | | | | | | | | | | | | |
| Phage- | L | D | N | W | — | K | — | S | — | — | — | T | — | — | 0.0569 | 2.0469 | 0.0633 | 2073 |
| 1777 | | | | | | | | | | | | | | | | | | |
| Phage- | E | F | K | W | — | I | — | S | — | — | — | H | E | T | 0.0817 | 2.0202 | 0.0857 | 2074 |
| 1778 | | | | | | | | | | | | | | | | | | |
| Phage- | V | V | K | A | — | I | — | T | — | — | — | Q | F | V | 0.0806 | 2.0184 | 0.0901 | 2075 |
| 1779 | | | | | | | | | | | | | | | | | | |
| Phage- | V | T | K | W | — | I | — | P | — | — | — | E | V | T | 0.0613 | 1.9988 | 0.0864 | 2076 |
| 1780 | | | | | | | | | | | | | | | | | | |
| Phage- | D | L | R | — | — | K | — | A | — | — | — | D | F | I | 0.0659 | 1.945 | 0.0601 | 2077 |
| 1781 | | | | | | | | | | | | | | | | | | |
| Phage- | L | D | K | W | — | V | — | K | — | — | — | S | — | L | 0.0595 | 1.9161 | 0.0742 | 2078 |
| 1782 | | | | | | | | | | | | | | | | | | |
| Phage- | G | E | K | W | — | I | — | D | — | — | — | S | — | — | 0.0609 | 1.9101 | 0.1476 | 2079 |
| 1783 | | | | | | | | | | | | | | | | | | |
| Phage- | L | E | K | T | — | I | — | T | — | — | — | V | — | — | 0.0552 | 1.8796 | 0.0732 | 2080 |
| 1784 | | | | | | | | | | | | | | | | | | |
| Phage- | V | E | K | — | — | T | — | I | — | — | — | L | — | — | 0.0504 | 1.8405 | 0.0705 | 2081 |
| 1785 | | | | | | | | | | | | | | | | | | |
| Phage- | D | V | K | — | — | K | — | E | — | — | — | Y | — | L | 0.0677 | 1.7541 | 0.0637 | 2082 |
| 1786 | | | | | | | | | | | | | | | | | | |
| Phage- | — | S | K | W | — | I | — | E | — | — | — | T | S | P | 0.0864 | 1.732 | 0.1074 | 2083 |
| 1787 | | | | | | | | | | | | | | | | | | |
| Phage- | L | E | K | — | — | K | — | A | — | — | — | V | N | — | 0.0742 | 1.6615 | 0.1071 | 2084 |
| 1788 | | | | | | | | | | | | | | | | | | |
| Phage- | — | D | I | R | — | T | — | I | — | — | — | V | — | — | 0.0546 | 1.5724 | 0.0801 | 2085 |
| 1789 | | | | | | | | | | | | | | | | | | |
| Phage- | E | P | K | W | — | V | — | R | G | L | — | N | Y | D | 0.0536 | 1.1395 | 0.0587 | 2086 |
| 1790 | | | | | | | | | | | | | | | | | | |
| Phage- | Y | F | — | Q | — | R | — | A | — | — | — | E | — | — | 0.168 | 2.5138 | 0.3386 | 2107 |
| 1791 | | | | | | | | | | | | | | | | | | |
| Phage- | — | V | P | — | — | K | — | A | — | — | — | K | L | — | 0.0747 | 2.48 | 0.2205 | 2108 |
| 1792 | | | | | | | | | | | | | | | | | | |
| Phage- | A | V | K | A | — | I | — | E | — | — | — | I | — | — | 0.0527 | 2.4411 | 0.1065 | 2109 |
| 1793 | | | | | | | | | | | | | | | | | | |
| Phage- | V | Q | I | Q | — | R | — | A | — | — | — | L | — | — | 0.0821 | 2.4399 | 0.1273 | 2110 |
| 1794 | | | | | | | | | | | | | | | | | | |
| Phage- | A | E | — | K | — | I | — | Q | — | — | — | Q | — | — | 0.0703 | 2.4261 | 0.0852 | 2111 |
| 1795 | | | | | | | | | | | | | | | | | | |
| Phage- | T | — | H | H | — | K | — | A | — | — | — | T | — | — | 0.0682 | 2.4226 | 0.1032 | 2112 |
| 1796 | | | | | | | | | | | | | | | | | | |
| Phage- | F | T | N | Y | — | V | — | S | — | — | — | T | — | — | 0.073 | 2.4218 | 0.1292 | 2113 |
| 1797 | | | | | | | | | | | | | | | | | | |
| Phage- | V | E | K | V | — | K | — | V | — | — | — | V | — | — | 0.0599 | 2.412 | 0.1557 | 2114 |
| 1798 | | | | | | | | | | | | | | | | | | |
| Phage- | E | E | P | — | — | R | — | V | — | — | — | E | — | — | 0.0612 | 2.4107 | 0.117 | 2115 |
| 1799 | | | | | | | | | | | | | | | | | | |
| Phage- | E | S | R | — | — | V | — | A | — | — | — | — | — | — | 0.0561 | 2.4088 | 0.1029 | 2116 |
| 1800 | | | | | | | | | | | | | | | | | | |
| Phage- | E | D | I | — | — | K | — | K | — | — | — | E | — | — | 0.0651 | 2.3955 | 0.1091 | 2117 |
| 1801 | | | | | | | | | | | | | | | | | | |
| Phage- | L | H | — | Y | — | V | — | E | — | — | — | — | — | — | 0.0577 | 2.3837 | 0.1094 | 2118 |
| 1802 | | | | | | | | | | | | | | | | | | |
| Phage- | L | E | P | — | — | V | — | R | — | — | — | V | — | — | 0.0702 | 2.3639 | 0.1115 | 2119 |
| 1803 | | | | | | | | | | | | | | | | | | |
| Phage- | — | T | P | V | — | K | — | L | — | — | — | S | — | L | 0.0518 | 2.3503 | 0.0764 | 2120 |
| 1804 | | | | | | | | | | | | | | | | | | |
| Phage- | V | E | — | K | — | F | — | L | — | F | — | V | — | L | 0.0613 | 2.2278 | 0.0923 | 2121 |
| 1805 | | | | | | | | | | | | | | | | | | |
| Phage- | — | T | P | I | — | K | — | A | — | I | — | S | — | — | 0.0542 | 2.1995 | 0.0631 | 2122 |
| 1806 | | | | | | | | | | | | | | | | | | |
| Phage- | V | V | I | K | — | K | — | E | — | — | — | S | — | — | 0.0667 | 2.1826 | 0.0946 | 2123 |
| 1807 | | | | | | | | | | | | | | | | | | |
| Phage- | P | E | H | — | — | L | — | R | — | — | — | E | — | — | 0.0638 | 1.7768 | 0.1347 | 2124 |
| 1808 | | | | | | | | | | | | | | | | | | |
| Phage- | Y | E | R | A | — | I | — | — | — | I | — | E | — | — | 0.0584 | 1.0347 | 0.0842 | 2125 |
| 1809 | | | | | | | | | | | | | | | | | | |
| Phage- | T | T | H | V | — | I | — | S | — | — | — | S | — | — | 0.0595 | 2.1457 | 0.0668 | 2126 |
| 1810 | | | | | | | | | | | | | | | | | | |
| Phage- | V | N | H | W | — | V | — | T | — | — | — | — | — | — | 0.1365 | 2.4053 | 0.186 | 2127 |
| 1811 | | | | | | | | | | | | | | | | | | |
| Phage- | — | V | — | K | — | V | — | S | — | — | — | E | — | — | 0.0556 | 2.4023 | 0.1024 | 2128 |
| 1812 | | | | | | | | | | | | | | | | | | |
| Phage- | T | V | A | Y | — | K | — | A | — | — | — | Q | — | — | 0.0622 | 2.3696 | 0.1082 | 2129 |
| 1813 | | | | | | | | | | | | | | | | | | |
| Phage- | T | E | — | R | — | T | — | P | — | — | — | L | — | — | 0.0779 | 2.3619 | 0.0981 | 2130 |
| 1814 | | | | | | | | | | | | | | | | | | |
| Phage- | V | E | R | — | — | T | — | A | — | — | — | H | — | — | 0.0678 | 2.358 | 0.2142 | 2131 |
| 1815 | | | | | | | | | | | | | | | | | | |
| Phage- | V | L | K | Y | — | I | — | D | — | — | — | — | — | I | 0.0931 | 2.2448 | 0.0978 | 2132 |
| 1816 | | | | | | | | | | | | | | | | | | |
| Phage- | P | E | R | — | — | T | — | P | — | — | — | L | — | — | 0.0738 | 2.2396 | 0.0917 | 2133 |
| 1817 | | | | | | | | | | | | | | | | | | |
| Phage- | M | E | R | W | — | V | — | D | — | — | — | — | T | — | 0.0594 | 2.4655 | 0.0578 | 2134 |
| 1818 | | | | | | | | | | | | | | | | | | |
| Phage- | A | L | K | — | — | I | — | F | — | — | — | E | — | L | 0.0608 | 2.4373 | 0.1286 | 2135 |
| 1819 | | | | | | | | | | | | | | | | | | |
| Phage- | M | V | K | W | — | T | — | A | — | — | — | E | M | L | 0.0845 | 2.412 | 0.1132 | 2136 |
| 1820 | | | | | | | | | | | | | | | | | | |
| Phage- | — | V | K | R | — | E | — | M | — | — | — | — | — | — | 0.0727 | 2.3766 | 0.2635 | 2137 |
| 1821 | | | | | | | | | | | | | | | | | | |
| Phage- | W | E | N | W | — | T | — | A | — | — | — | V | — | — | 0.0849 | 2.3708 | 0.0932 | 2138 |
| 1822 | | | | | | | | | | | | | | | | | | |
| Phage- | T | E | I | R | — | T | — | Q | — | — | — | L | — | — | 0.0655 | 2.3701 | 0.0871 | 2139 |
| 1823 | | | | | | | | | | | | | | | | | | |
| Phage- | V | E | K | M | — | R | — | A | — | — | — | W | V | M | 0.147 | 2.365 | 0.2138 | 2140 |
| 1824 | | | | | | | | | | | | | | | | | | |
| Phage- | — | S | K | — | — | T | — | P | — | — | — | E | M | L | 0.0517 | 2.3305 | 0.0582 | 2141 |
| 1825 | | | | | | | | | | | | | | | | | | |
| Phage- | W | E | N | W | — | — | — | A | — | — | — | T | — | L | 0.0618 | 2.3297 | 0.0691 | 2142 |
| 1826 | | | | | | | | | | | | | | | | | | |
| Phage- | V | L | K | — | — | V | — | P | — | — | — | M | A | Q | 0.0773 | 2.319 | 0.0968 | 2143 |
| 1827 | | | | | | | | | | | | | | | | | | |
| Phage- | M | D | N | W | — | T | — | E | — | — | — | L | — | — | 0.0593 | 2.3025 | 0.065 | 2144 |
| 1828 | | | | | | | | | | | | | | | | | | |
| Phage- | M | D | K | W | — | T | — | P | — | — | — | D | W | L | 0.0562 | 2.2999 | 0.1361 | 2145 |
| 1829 | | | | | | | | | | | | | | | | | | |
| Phage- | E | P | K | W | — | T | — | A | — | — | — | E | W | M | 0.0584 | 2.2935 | 0.0904 | 2146 |
| 1830 | | | | | | | | | | | | | | | | | | |
| Phage- | — | I | P | W | — | K | — | A | — | — | — | S | — | M | 0.1454 | 2.2879 | 0.2751 | 2147 |
| 1831 | | | | | | | | | | | | | | | | | | |
| Phage- | M | G | K | W | — | I | — | E | — | — | — | N | V | S | 0.0602 | 2.2856 | 0.0652 | 2148 |
| 1832 | | | | | | | | | | | | | | | | | | |
| Phage- | D | I | P | W | — | I | — | P | — | — | — | — | — | S | 0.0679 | 2.2782 | 0.1118 | 2149 |
| 1833 | | | | | | | | | | | | | | | | | | |
| Phage- | V | E | R | M | — | M | — | A | — | — | — | D | M | — | 0.0824 | 2.2773 | 0.0862 | 2150 |
| 1834 | | | | | | | | | | | | | | | | | | |
| Phage- | Y | E | K | W | — | V | — | D | — | — | — | S | M | I | 0.059 | 2.2766 | 0.0678 | 2151 |
| 1835 | | | | | | | | | | | | | | | | | | |
| Phage- | W | L | P | M | — | R | — | A | — | — | — | — | — | L | 0.0582 | 2.2727 | 0.0653 | 2152 |
| 1836 | | | | | | | | | | | | | | | | | | |
| Phage- | G | L | N | W | — | T | — | T | — | — | — | Q | — | — | 0.0551 | 2.2615 | 0.0833 | 2153 |
| 1837 | | | | | | | | | | | | | | | | | | |
| Phage- | V | M | K | W | — | I | — | V | — | — | — | G | — | M | 0.1088 | 2.2576 | 0.2914 | 2154 |
| 1838 | | | | | | | | | | | | | | | | | | |
| Phage- | Y | D | I | — | — | R | — | S | — | — | — | S | W | V | 0.0739 | 2.2438 | 0.145 | 2155 |
| 1839 | | | | | | | | | | | | | | | | | | |
| Phage- | M | V | K | W | — | I | — | S | — | — | — | D | S | V | 0.1266 | 2.2324 | 0.1088 | 2156 |
| 1840 | | | | | | | | | | | | | | | | | | |
| Phage- | T | D | K | W | — | I | — | P | — | — | — | M | L | Q | 0.0701 | 2.227 | 0.0865 | 2157 |
| 1841 | | | | | | | | | | | | | | | | | | |
| Phage- | V | E | R | W | — | V | — | E | — | — | — | H | W | I | 0.072 | 2.2268 | 0.0977 | 2158 |
| 1842 | | | | | | | | | | | | | | | | | | |
| Phage- | L | D | R | W | — | I | — | E | — | — | — | E | M | — | 0.0618 | 2.2204 | 0.0928 | 2159 |
| 1843 | | | | | | | | | | | | | | | | | | |
| Phage- | T | D | K | — | — | I | — | E | — | — | — | I | N | W | 0.061 | 2.2201 | 0.0602 | 2160 |
| 1844 | | | | | | | | | | | | | | | | | | |
| Phage- | F | T | N | W | — | T | — | E | — | — | — | S | — | — | 0.0575 | 2.2135 | 0.0826 | 2161 |
| 1845 | | | | | | | | | | | | | | | | | | |
| Phage- | P | S | M | R | — | I | — | E | — | — | — | — | M | L | 0.0607 | 2.2019 | 0.0824 | 2162 |
| 1846 | | | | | | | | | | | | | | | | | | |
| Phage- | S | M | H | W | — | T | — | K | — | — | — | E | — | L | 0.088 | 2.1979 | 0.1473 | 2163 |
| 1847 | | | | | | | | | | | | | | | | | | |
| Phage- | A | I | P | W | — | I | — | A | — | — | — | Q | V | — | 0.1841 | 2.1845 | 0.2337 | 2164 |
| 1848 | | | | | | | | | | | | | | | | | | |
| Phage- | F | S | H | W | — | I | — | E | — | — | — | S | T | S | 0.1015 | 2.1763 | 0.1582 | 2165 |
| 1849 | | | | | | | | | | | | | | | | | | |
| Phage- | P | T | K | — | — | I | — | E | — | — | — | Y | V | R | 0.0544 | 2.1736 | 0.0692 | 2166 |
| 1850 | | | | | | | | | | | | | | | | | | |
| Phage- | S | E | M | — | — | K | — | S | — | — | — | L | — | — | 0.0574 | 2.1735 | 0.0726 | 2167 |
| 1851 | | | | | | | | | | | | | | | | | | |
| Phage- | W | D | H | W | — | T | — | A | — | — | — | E | F | V | 0.0566 | 2.1713 | 0.0692 | 2168 |
| 1852 | | | | | | | | | | | | | | | | | | |
| Phage- | M | E | K | W | — | I | — | E | — | — | — | L | H | T | 0.0686 | 2.1688 | 0.0816 | 2169 |
| 1853 | | | | | | | | | | | | | | | | | | |
| Phage- | W | E | K | W | — | T | — | Q | — | — | — | E | L | I | 0.1161 | 2.167 | 0.1824 | 2170 |
| 1854 | | | | | | | | | | | | | | | | | | |
| Phage- | M | E | K | W | — | T | — | S | — | — | — | V | — | — | 0.0777 | 2.165 | 0.1483 | 2171 |
| 1855 | | | | | | | | | | | | | | | | | | |
| Phage- | M | G | N | W | — | I | — | T | — | — | — | T | — | — | 0.0985 | 2.1646 | 0.1618 | 2172 |
| 1856 | | | | | | | | | | | | | | | | | | |
| Phage- | V | M | K | W | — | I | — | S | — | — | — | S | Q | D | 0.1088 | 2.1633 | 0.1651 | 2173 |
| 1857 | | | | | | | | | | | | | | | | | | |
| Phage- | V | D | K | W | — | T | — | I | — | — | — | E | — | — | 0.0683 | 2.1593 | 0.0951 | 2174 |
| 1858 | | | | | | | | | | | | | | | | | | |
| Phage- | V | E | K | W | — | V | — | T | — | — | — | D | W | V | 0.0674 | 2.1585 | 0.0833 | 2175 |
| 1859 | | | | | | | | | | | | | | | | | | |
| Phage- | G | D | N | W | — | K | — | E | — | — | — | I | — | — | 0.0992 | 2.1579 | 0.0978 | 2176 |
| 1860 | | | | | | | | | | | | | | | | | | |
| Phage- | A | V | K | — | — | I | — | E | — | — | — | V | — | Q | 0.0645 | 2.1495 | 0.0676 | 2177 |
| 1861 | | | | | | | | | | | | | | | | | | |
| Phage- | L | D | K | W | — | I | — | E | — | — | — | F | D | Y | 0.0607 | 2.1438 | 0.0678 | 2178 |
| 1862 | | | | | | | | | | | | | | | | | | |
| Phage- | D | F | R | W | — | I | — | E | — | — | — | T | — | Y | 0.0625 | 2.1413 | 0.1097 | 2179 |
| 1863 | | | | | | | | | | | | | | | | | | |
| Phage- | — | M | K | W | — | I | — | E | — | — | — | Y | F | — | 0.0664 | 2.1398 | 0.1 | 2180 |
| 1864 | | | | | | | | | | | | | | | | | | |
| Phage- | E | I | T | R | — | I | — | A | — | — | — | D | F | W | 0.0626 | 2.1289 | 0.081 | 2181 |
| 1865 | | | | | | | | | | | | | | | | | | |
| Phage- | W | E | H | W | — | R | — | A | — | — | — | E | S | L | 0.1163 | 2.1233 | 0.1341 | 2182 |
| 1866 | | | | | | | | | | | | | | | | | | |
| Phage- | W | E | N | R | — | T | — | A | — | — | — | K | — | — | 0.0516 | 2.1204 | 0.0837 | 2183 |
| 1867 | | | | | | | | | | | | | | | | | | |
| Phage- | M | E | K | — | — | K | — | A | — | — | — | S | M | L | 0.056 | 2.1138 | 0.0627 | 2184 |
| 1868 | | | | | | | | | | | | | | | | | | |
| Phage- | S | I | K | M | — | I | — | Q | — | — | — | V | M | Q | 0.1003 | 2.1113 | 0.1787 | 2185 |
| 1869 | | | | | | | | | | | | | | | | | | |
| Phage- | E | F | I | W | — | R | — | A | — | — | — | M | D | V | 0.063 | 2.1054 | 0.0681 | 2186 |
| 1870 | | | | | | | | | | | | | | | | | | |
| Phage- | H | V | K | W | — | I | — | A | — | — | — | E | A | S | 0.0687 | 2.1032 | 0.0972 | 2187 |
| 1871 | | | | | | | | | | | | | | | | | | |
| Phage- | V | M | K | W | — | I | — | E | — | — | — | Q | H | T | 0.0897 | 2.0938 | 0.1991 | 2188 |
| 1872 | | | | | | | | | | | | | | | | | | |
| Phage- | V | E | H | W | — | I | — | A | — | — | — | E | W | M | 0.0668 | 2.0905 | 0.1196 | 2189 |
| 1873 | | | | | | | | | | | | | | | | | | |
| Phage- | S | P | K | W | — | T | — | E | — | — | — | S | H | L | 0.0658 | 2.0884 | 0.127 | 2190 |
| 1874 | | | | | | | | | | | | | | | | | | |
| Phage- | F | V | P | W | — | I | — | A | — | — | — | — | E | N | 0.0907 | 2.0848 | 0.1111 | 2191 |
| 1875 | | | | | | | | | | | | | | | | | | |
| Phage- | V | V | K | — | — | I | — | E | — | — | — | — | E | W | 0.0666 | 2.0838 | 0.1149 | 2192 |
| 1876 | | | | | | | | | | | | | | | | | | |
| Phage- | — | E | R | W | — | I | — | D | — | — | — | H | V | W | 0.0805 | 2.0772 | 0.1257 | 2193 |
| 1877 | | | | | | | | | | | | | | | | | | |
| Phage- | V | E | I | — | — | K | — | K | — | — | — | E | F | W | 0.082 | 2.0697 | 0.1091 | 2194 |
| 1878 | | | | | | | | | | | | | | | | | | |
| Phage- | S | L | H | W | — | I | — | A | — | — | — | D | F | V | 0.1363 | 2.0666 | 0.1853 | 2195 |
| 1879 | | | | | | | | | | | | | | | | | | |
| Phage- | V | L | M | — | — | K | — | A | — | — | — | M | — | — | 0.1882 | 2.0662 | 0.3749 | 2196 |
| 1880 | | | | | | | | | | | | | | | | | | |
| Phage- | L | E | K | W | — | T | — | A | — | — | — | E | M | L | 0.065 | 2.0648 | 0.0814 | 2197 |
| 1881 | | | | | | | | | | | | | | | | | | |
| Phage- | V | T | P | W | — | T | — | S | — | — | — | F | — | — | 0.0683 | 2.0627 | 0.072 | 2198 |
| 1882 | | | | | | | | | | | | | | | | | | |
| Phage- | V | N | K | — | — | T | — | E | — | — | — | V | — | M | 0.0732 | 2.0604 | 0.0609 | 2199 |
| 1883 | | | | | | | | | | | | | | | | | | |
| Phage- | M | V | M | R | — | — | — | E | — | — | — | T | — | I | 0.0641 | 2.0588 | 0.1224 | 2200 |
| 1884 | | | | | | | | | | | | | | | | | | |
| Phage- | F | — | K | W | — | I | — | E | — | — | — | S | V | S | 0.1115 | 2.0551 | 0.1194 | 2201 |
| 1885 | | | | | | | | | | | | | | | | | | |
| Phage- | W | E | K | W | — | I | — | A | — | — | — | V | A | D | 0.0912 | 2.0533 | 0.1342 | 2202 |
| 1886 | | | | | | | | | | | | | | | | | | |
| Phage- | L | D | H | W | — | F | — | M | — | — | — | T | — | — | 0.139 | 2.0508 | 0.2334 | 2203 |
| 1887 | | | | | | | | | | | | | | | | | | |
| Phage- | W | S | N | W | — | T | — | E | — | — | — | V | — | — | 0.0678 | 2.0492 | 0.0814 | 2204 |
| 1888 | | | | | | | | | | | | | | | | | | |
| Phage- | D | V | M | W | — | K | — | S | — | — | — | D | W | L | 0.0852 | 2.0442 | 0.1271 | 2205 |
| 1889 | | | | | | | | | | | | | | | | | | |
| Phage- | A | I | K | W | — | I | — | S | — | — | — | V | E | D | 0.0651 | 2.0432 | 0.0876 | 2206 |
| 1890 | | | | | | | | | | | | | | | | | | |
| Phage- | L | E | K | W | — | I | — | A | — | — | — | E | W | S | 0.0676 | 2.0418 | 0.1129 | 2207 |
| 1891 | | | | | | | | | | | | | | | | | | |
| Phage- | V | P | K | — | — | T | — | V | — | — | — | T | — | — | 0.0596 | 2.0416 | 0.0783 | 2208 |
| 1892 | | | | | | | | | | | | | | | | | | |
| Phage- | F | D | K | W | — | I | — | P | — | — | — | Q | A | L | 0.0832 | 2.0393 | 0.112 | 2209 |
| 1893 | | | | | | | | | | | | | | | | | | |
| Phage- | F | E | K | W | — | T | — | A | — | — | — | — | — | W | 0.0692 | 2.0354 | 0.0792 | 2210 |
| 1894 | | | | | | | | | | | | | | | | | | |
| Phage- | W | E | K | M | — | V | — | S | — | — | — | H | F | S | 0.0647 | 2.0349 | 0.0735 | 2211 |
| 1895 | | | | | | | | | | | | | | | | | | |
| Phage- | V | L | K | W | — | T | — | A | — | — | — | E | I | — | 0.16 | 2.034 | 0.1413 | 2212 |
| 1896 | | | | | | | | | | | | | | | | | | |
| Phage- | M | E | K | — | — | I | — | A | — | — | — | D | — | M | 0.056 | 2.033 | 0.2794 | 2213 |
| 1897 | | | | | | | | | | | | | | | | | | |
| Phage- | A | E | K | W | — | F | — | A | — | — | — | E | R | P | 0.0777 | 2.0322 | 0.0803 | 2214 |
| 1898 | | | | | | | | | | | | | | | | | | |
| Phage- | S | V | K | M | — | T | — | A | — | — | — | S | — | — | 0.0546 | 2.0279 | 0.0752 | 2215 |
| 1899 | | | | | | | | | | | | | | | | | | |
| Phage- | V | S | R | — | — | I | — | T | — | — | — | D | Y | S | 0.0642 | 2.0269 | 0.0757 | 2216 |
| 1900 | | | | | | | | | | | | | | | | | | |
| Phage- | T | D | M | W | — | T | — | M | — | — | — | M | — | — | 0.0612 | 2.0257 | 0.0829 | 2217 |
| 1901 | | | | | | | | | | | | | | | | | | |
| Phage- | L | I | P | W | — | I | — | A | — | — | — | T | S | D | 0.0836 | 2.0256 | 0.0863 | 2218 |
| 1902 | | | | | | | | | | | | | | | | | | |
| Phage- | — | D | M | R | — | T | — | — | — | — | — | I | — | — | 0.067 | 2.0252 | 0.081 | 2219 |
| 1903 | | | | | | | | | | | | | | | | | | |
| Phage- | D | V | K | W | — | I | — | A | — | — | — | E | — | — | 0.0523 | 2.0237 | 0.1054 | 2220 |
| 1904 | | | | | | | | | | | | | | | | | | |
| Phage- | E | I | R | — | — | T | — | S | — | — | — | M | — | — | 0.0599 | 2.0228 | 0.0792 | 2221 |
| 1905 | | | | | | | | | | | | | | | | | | |
| Phage- | V | M | K | W | — | I | — | E | — | — | — | Y | M | L | 0.1189 | 2.0212 | 0.2038 | 2222 |
| 1906 | | | | | | | | | | | | | | | | | | |
| Phage- | S | — | H | M | — | I | — | P | — | — | — | L | H | T | 0.093 | 2.018 | 0.1332 | 2223 |
| 1907 | | | | | | | | | | | | | | | | | | |
| Phage- | W | D | N | W | — | I | — | P | — | — | — | I | — | — | 0.1032 | 2.0168 | 0.1865 | 2224 |
| 1908 | | | | | | | | | | | | | | | | | | |
| Phage- | M | M | K | W | — | T | — | A | — | — | — | E | E | A | 0.0581 | 2.0105 | 0.0691 | 2225 |
| 1909 | | | | | | | | | | | | | | | | | | |
| Phage- | G | D | I | — | — | R | — | E | — | — | — | T | — | — | 0.063 | 2.0098 | 0.1114 | 2226 |
| 1910 | | | | | | | | | | | | | | | | | | |
| Phage- | G | D | P | W | — | I | — | A | — | — | — | M | E | — | 0.0631 | 2.0059 | 0.088 | 2227 |
| 1911 | | | | | | | | | | | | | | | | | | |
| Phage- | M | M | K | W | — | I | — | T | — | — | — | E | M | E | 0.0797 | 2.0038 | 0.1316 | 2228 |
| 1912 | | | | | | | | | | | | | | | | | | |
| Phage- | E | S | I | R | — | I | — | L | — | — | — | E | — | — | 0.0635 | 2.0024 | 0.0894 | 2229 |
| 1913 | | | | | | | | | | | | | | | | | | |
| Phage- | V | I | K | W | — | T | — | A | — | — | — | T | A | L | 0.07 | 2.0004 | 0.0733 | 2230 |
| 1914 | | | | | | | | | | | | | | | | | | |
| Phage- | S | F | A | W | — | I | — | P | — | — | — | E | I | T | 0.0795 | 1.998 | 0.1273 | 2231 |
| 1915 | | | | | | | | | | | | | | | | | | |
| Phage- | F | T | I | W | — | I | — | E | — | — | — | L | H | E | 0.0896 | 1.9979 | 0.0999 | 2232 |
| 1916 | | | | | | | | | | | | | | | | | | |
| Phage- | — | E | K | M | — | K | — | A | — | — | — | W | A | D | 0.0726 | 1.9972 | 0.0706 | 2233 |
| 1917 | | | | | | | | | | | | | | | | | | |
| Phage- | E | K | I | W | — | V | — | A | — | — | — | T | W | L | 0.1023 | 1.9943 | 0.1213 | 2234 |
| 1918 | | | | | | | | | | | | | | | | | | |
| Phage- | V | S | K | — | — | I | — | M | — | — | — | E | T | S | 0.0544 | 1.994 | 0.064 | 2235 |
| 1919 | | | | | | | | | | | | | | | | | | |
| Phage- | G | E | Y | W | — | I | — | S | — | — | — | Q | L | Y | 0.1327 | 1.9935 | 0.1786 | 2236 |
| 1920 | | | | | | | | | | | | | | | | | | |
| Phage- | V | D | R | W | — | V | — | V | — | — | — | E | — | L | 0.0516 | 1.9915 | 0.1239 | 2237 |
| 1921 | | | | | | | | | | | | | | | | | | |
| Phage- | L | V | N | W | — | I | — | M | — | — | — | E | — | — | 0.0699 | 1.99 | 0.1071 | 2238 |
| 1922 | | | | | | | | | | | | | | | | | | |
| Phage- | V | E | K | W | — | I | — | T | — | — | — | E | L | S | 0.0626 | 1.989 | 0.0767 | 2239 |
| 1923 | | | | | | | | | | | | | | | | | | |
| Phage- | T | E | K | — | — | I | — | E | — | — | — | F | — | M | 0.0583 | 1.9868 | 0.0644 | 2240 |
| 1924 | | | | | | | | | | | | | | | | | | |
| Phage- | T | E | M | W | — | K | — | A | — | — | — | T | — | — | 0.0578 | 1.9859 | 0.0938 | 2241 |
| 1925 | | | | | | | | | | | | | | | | | | |
| Phage- | F | — | K | W | — | T | — | E | — | — | — | V | — | — | 0.0646 | 1.9857 | 0.125 | 2242 |
| 1926 | | | | | | | | | | | | | | | | | | |
| Phage- | W | E | N | W | — | T | — | A | — | — | — | S | H | M | 0.0713 | 1.9836 | 0.1002 | 2243 |
| 1927 | | | | | | | | | | | | | | | | | | |
| Phage- | E | L | H | — | — | I | — | R | G | — | — | K | F | R | 0.0539 | 1.9799 | 0.0668 | 2244 |
| 1928 | | | | | | | | | | | | | | | | | | |
| Phage- | A | E | K | W | — | I | — | A | — | — | — | E | A | M | 0.0661 | 1.9779 | 0.0668 | 2245 |
| 1929 | | | | | | | | | | | | | | | | | | |
| Phage- | V | Q | N | W | — | I | — | A | — | — | — | Q | — | M | 0.0605 | 1.9756 | 0.0574 | 2246 |
| 1930 | | | | | | | | | | | | | | | | | | |
| Phage- | W | D | R | — | — | T | — | A | — | — | — | T | — | — | 0.0714 | 1.971 | 0.104 | 2247 |
| 1931 | | | | | | | | | | | | | | | | | | |
| Phage- | D | F | K | W | — | T | — | M | — | — | — | E | H | L | 0.0816 | 1.9708 | 0.1062 | 2248 |
| 1932 | | | | | | | | | | | | | | | | | | |
| Phage- | V | V | Q | W | — | I | — | A | — | — | — | — | A | Q | 0.0708 | 1.9667 | 0.0955 | 2249 |
| 1933 | | | | | | | | | | | | | | | | | | |
| Phage- | V | S | I | W | — | R | — | A | — | — | — | E | S | G | 0.0796 | 1.9656 | 0.0809 | 2250 |
| 1934 | | | | | | | | | | | | | | | | | | |
| Phage- | V | E | K | — | — | K | — | I | — | — | — | N | W | Q | 0.0592 | 1.96 | 0.0992 | 2251 |
| 1935 | | | | | | | | | | | | | | | | | | |
| Phage- | W | P | K | W | — | T | — | P | — | — | — | F | Q | D | 0.1158 | 1.9596 | 0.1483 | 2252 |
| 1936 | | | | | | | | | | | | | | | | | | |
| Phage- | F | P | K | W | — | T | — | A | — | — | — | E | R | S | 0.0703 | 1.9589 | 0.0909 | 2253 |
| 1937 | | | | | | | | | | | | | | | | | | |
| Phage- | M | E | K | W | — | I | — | S | — | — | — | D | V | N | 0.0732 | 1.9562 | 0.0785 | 2254 |
| 1938 | | | | | | | | | | | | | | | | | | |
| Phage- | V | E | K | — | — | R | — | S | — | — | — | E | H | M | 0.0612 | 1.9553 | 0.0564 | 2255 |
| 1939 | | | | | | | | | | | | | | | | | | |
| Phage- | W | E | N | W | — | R | — | E | — | — | — | T | — | L | 0.0673 | 1.9547 | 0.0907 | 2256 |
| 1940 | | | | | | | | | | | | | | | | | | |
| Phage- | G | E | K | W | — | I | — | E | — | — | — | T | T | W | 0.0525 | 1.9521 | 0.1347 | 2257 |
| 1941 | | | | | | | | | | | | | | | | | | |
| Phage- | V | E | K | W | — | V | — | Q | — | — | — | G | Q | M | 0.0644 | 1.9512 | 0.0869 | 2258 |
| 1942 | | | | | | | | | | | | | | | | | | |
| Phage- | L | E | K | W | — | I | — | T | — | — | — | E | L | — | 0.1247 | 1.9432 | 0.1752 | 2259 |
| 1943 | | | | | | | | | | | | | | | | | | |
| Phage- | D | G | K | W | — | I | — | P | — | — | — | Q | S | T | 0.0697 | 1.9396 | 0.077 | 2260 |
| 1944 | | | | | | | | | | | | | | | | | | |
| Phage- | W | E | N | W | — | T | — | R | — | — | — | E | — | — | 0.0592 | 1.9395 | 0.1732 | 2261 |
| 1945 | | | | | | | | | | | | | | | | | | |
| Phage- | T | E | M | W | — | K | — | S | — | — | — | Y | — | — | 0.0607 | 1.9372 | 0.1012 | 2262 |
| 1946 | | | | | | | | | | | | | | | | | | |
| Phage- | W | I | N | W | — | T | — | E | — | — | — | M | — | — | 0.1642 | 1.9361 | 0.2399 | 2263 |
| 1947 | | | | | | | | | | | | | | | | | | |
| Phage- | M | D | N | W | — | T | — | S | — | — | — | V | — | — | 0.0533 | 1.9345 | 0.0794 | 2264 |
| 1948 | | | | | | | | | | | | | | | | | | |
| Phage- | D | I | K | — | — | M | — | E | — | — | — | L | — | — | 0.0683 | 1.9342 | 0.0792 | 2265 |
| 1949 | | | | | | | | | | | | | | | | | | |
| Phage- | A | R | R | S | — | V | F | R | R | G | — | V | F | M | 0.0479 | 1.9328 | 0.0701 | 2266 |
| 1950 | | | | | | | | | | | | | | | | | | |
| Phage- | A | E | H | W | — | I | — | S | — | — | — | — | — | — | 0.0605 | 1.9318 | 0.1043 | 2267 |
| 1951 | | | | | | | | | | | | | | | | | | |
| Phage- | M | P | K | W | — | T | — | A | — | — | — | — | H | Q | 0.083 | 1.9292 | 0.0835 | 2268 |
| 1952 | | | | | | | | | | | | | | | | | | |
| Phage- | P | V | K | W | — | I | — | T | — | — | — | — | P | V | 0.0686 | 1.9242 | 0.0978 | 2269 |
| 1953 | | | | | | | | | | | | | | | | | | |
| Phage- | H | E | K | W | — | I | — | E | — | — | — | V | V | T | 0.0538 | 1.921 | 0.0642 | 2270 |
| 1954 | | | | | | | | | | | | | | | | | | |
| Phage- | G | E | I | W | — | I | — | P | — | — | — | Y | A | P | 0.0749 | 1.9208 | 0.0825 | 2271 |
| 1955 | | | | | | | | | | | | | | | | | | |
| Phage- | F | — | K | W | — | T | — | V | — | — | — | — | — | L | 0.1906 | 1.9196 | 0.2806 | 2272 |
| 1956 | | | | | | | | | | | | | | | | | | |
| Phage- | L | E | N | W | — | I | — | A | — | — | — | S | — | — | 0.0832 | 1.9192 | 0.0656 | 2273 |
| 1957 | | | | | | | | | | | | | | | | | | |
| Phage- | D | G | K | W | — | I | — | A | — | — | — | V | D | M | 0.0578 | 1.9185 | 0.0672 | 2274 |
| 1958 | | | | | | | | | | | | | | | | | | |
| Phage- | W | D | K | W | — | I | — | E | — | — | — | E | S | M | 0.0855 | 1.9153 | 0.0718 | 2275 |
| 1959 | | | | | | | | | | | | | | | | | | |
| Phage- | F | E | K | W | — | I | — | D | — | — | — | Q | V | M | 0.0912 | 1.9141 | 0.0817 | 2276 |
| 1960 | | | | | | | | | | | | | | | | | | |
| Phage- | E | G | I | W | — | T | — | A | — | — | — | M | — | — | 0.0619 | 1.9137 | 0.0798 | 2277 |
| 1961 | | | | | | | | | | | | | | | | | | |
| Phage- | V | V | K | W | — | L | — | P | — | — | — | T | D | — | 0.0557 | 1.9134 | 0.0829 | 2278 |
| 1962 | | | | | | | | | | | | | | | | | | |
| Phage- | L | V | K | W | — | T | — | A | — | — | — | E | F | I | 0.1063 | 1.9118 | 0.0856 | 2279 |
| 1963 | | | | | | | | | | | | | | | | | | |
| Phage- | L | E | M | W | — | V | — | A | — | — | — | S | — | — | 0.0561 | 1.9101 | 0.0771 | 2280 |
| 1964 | | | | | | | | | | | | | | | | | | |
| Phage- | M | V | K | W | — | K | — | A | — | — | — | D | V | I | 0.0641 | 1.9096 | 0.078 | 2281 |
| 1965 | | | | | | | | | | | | | | | | | | |
| Phage- | W | D | K | W | — | R | — | A | — | — | — | E | S | T | 0.1056 | 1.9079 | 0.0851 | 2282 |
| 1966 | | | | | | | | | | | | | | | | | | |
| Phage- | D | V | K | M | — | I | — | A | — | — | — | S | F | D | 0.0602 | 1.9052 | 0.0712 | 2283 |
| 1967 | | | | | | | | | | | | | | | | | | |
| Phage- | Y | I | K | W | — | T | — | S | — | — | — | E | A | S | 0.0812 | 1.903 | 0.1128 | 2284 |
| 1968 | | | | | | | | | | | | | | | | | | |
| Phage- | V | E | K | W | — | K | — | T | — | — | — | E | M | V | 0.0799 | 1.9023 | 0.0976 | 2285 |
| 1969 | | | | | | | | | | | | | | | | | | |
| Phage- | E | F | K | W | — | T | — | S | — | — | — | — | H | L | 0.0633 | 1.8995 | 0.1284 | 2286 |
| 1970 | | | | | | | | | | | | | | | | | | |
| Phage- | V | M | N | W | — | T | — | V | — | — | — | Q | — | — | 0.0744 | 1.899 | 0.1153 | 2287 |
| 1971 | | | | | | | | | | | | | | | | | | |
| Phage- | P | W | H | W | — | I | — | E | — | — | — | — | E | Y | 0.0975 | 1.8982 | 0.083 | 2288 |
| 1972 | | | | | | | | | | | | | | | | | | |
| Phage- | F | D | K | W | — | I | — | E | — | — | — | — | V | L | 0.0912 | 1.8964 | 0.0682 | 2289 |
| 1973 | | | | | | | | | | | | | | | | | | |
| Phage- | L | V | K | W | — | I | — | E | — | — | — | E | V | L | 0.15 | 1.8953 | 0.0751 | 2290 |
| 1974 | | | | | | | | | | | | | | | | | | |
| Phage- | F | D | N | W | — | K | — | S | — | — | — | — | — | L | 0.0774 | 1.8932 | 0.0863 | 2291 |
| 1975 | | | | | | | | | | | | | | | | | | |
| Phage- | T | M | H | W | — | I | — | Q | — | — | — | E | T | G | 0.0671 | 1.893 | 0.2503 | 2292 |
| 1976 | | | | | | | | | | | | | | | | | | |
| Phage- | M | — | Q | W | — | T | — | A | — | — | — | S | — | — | 0.0827 | 1.8924 | 0.0721 | 2293 |
| 1977 | | | | | | | | | | | | | | | | | | |
| Phage- | S | E | R | W | — | I | — | T | — | — | — | Q | E | T | 0.0629 | 1.8921 | 0.0819 | 2294 |
| 1978 | | | | | | | | | | | | | | | | | | |
| Phage- | L | P | K | W | — | T | — | E | — | — | — | I | — | — | 0.0639 | 1.8902 | 0.1131 | 2295 |
| 1979 | | | | | | | | | | | | | | | | | | |
| Phage- | L | D | K | W | — | I | — | P | — | — | — | Y | S | I | 0.0569 | 1.8855 | 0.0602 | 2296 |
| 1980 | | | | | | | | | | | | | | | | | | |
| Phage- | p | P | I | — | — | I | — | Q | — | — | — | S | P | T | 0.0599 | 1.8849 | 0.0785 | 2297 |
| 1981 | | | | | | | | | | | | | | | | | | |
| Phage- | V | G | M | — | — | K | — | S | — | — | — | D | W | L | 0.0628 | 1.8833 | 0.0813 | 2298 |
| 1982 | | | | | | | | | | | | | | | | | | |
| Phage- | — | F | E | W | — | V | — | A | — | — | — | E | S | R | 0.0633 | 1.8831 | 0.1036 | 2299 |
| 1983 | | | | | | | | | | | | | | | | | | |
| Phage- | V | V | K | M | — | R | — | A | — | — | — | D | L | V | 0.0621 | 1.8822 | 0.0833 | 2300 |
| 1984 | | | | | | | | | | | | | | | | | | |
| Phage- | L | E | N | W | — | I | — | R | — | — | — | H | — | — | 0.1694 | 1.8813 | 0.1522 | 2301 |
| 1985 | | | | | | | | | | | | | | | | | | |
| Phage- | Y | E | K | W | — | T | — | P | — | — | — | E | M | L | 0.0668 | 1.8788 | 0.0833 | 2302 |
| 1986 | | | | | | | | | | | | | | | | | | |
| Phage- | V | E | K | W | — | T | — | P | — | — | — | — | — | W | 0.0563 | 1.8746 | 0.0659 | 2303 |
| 1987 | | | | | | | | | | | | | | | | | | |
| Phage- | L | D | N | W | E | R | — | L | — | — | — | T | — | — | 0.0727 | 1.873 | 0.0836 | 2304 |
| 1988 | | | | | | | | | | | | | | | | | | |
| Phage- | E | P | K | — | — | T | — | T | — | — | — | I | — | — | 0.0648 | 1.8685 | 0.1176 | 2305 |
| 1989 | | | | | | | | | | | | | | | | | | |
| Phage- | L | M | K | W | — | I | — | E | — | — | — | V | D | S | 0.0577 | 1.8679 | 0.0631 | 2306 |
| 1990 | | | | | | | | | | | | | | | | | | |
| Phage- | L | V | K | W | — | T | — | S | — | — | — | I | N | L | 0.0984 | 1.8656 | 0.0883 | 2307 |
| 1991 | | | | | | | | | | | | | | | | | | |
| Phage- | P | V | K | W | — | I | — | D | — | — | — | — | Y | L | 0.069 | 1.8629 | 0.1149 | 2308 |
| 1992 | | | | | | | | | | | | | | | | | | |
| Phage- | E | V | K | W | — | T | — | P | — | — | — | M | F | D | 0.0621 | 1.8628 | 0.073 | 2309 |
| 1993 | | | | | | | | | | | | | | | | | | |
| Phage- | F | D | K | W | — | I | — | T | — | — | — | E | L | V | 0.0636 | 1.8624 | 0.1452 | 2310 |
| 1994 | | | | | | | | | | | | | | | | | | |
| Phage- | M | G | K | W | — | I | — | P | — | — | — | V | V | D | 0.0917 | 1.8604 | 0.1232 | 2311 |
| 1995 | | | | | | | | | | | | | | | | | | |
| Phage- | G | S | N | W | — | K | — | A | — | — | — | E | I | M | 0.0585 | 1.8597 | 0.0723 | 2312 |
| 1996 | | | | | | | | | | | | | | | | | | |
| Phage- | N | F | H | — | — | T | — | Q | — | — | — | T | — | — | 0.0685 | 1.8557 | 0.0973 | 2313 |
| 1997 | | | | | | | | | | | | | | | | | | |
| Phage- | — | D | K | W | — | I | — | A | — | — | — | V | S | N | 0.0841 | 1.8553 | 0.0815 | 2314 |
| 1998 | | | | | | | | | | | | | | | | | | |
| Phage- | — | P | K | W | — | T | — | A | — | — | — | E | S | M | 0.0575 | 1.8552 | 0.0609 | 2315 |
| 1999 | | | | | | | | | | | | | | | | | | |
| Phage- | H | E | M | W | — | R | — | A | — | — | — | D | — | — | 0.0509 | 1.8521 | 0.0635 | 2316 |
| 2000 | | | | | | | | | | | | | | | | | | |
| Phage- | E | V | K | W | — | T | — | A | — | — | — | E | — | — | 0.054 | 1.8518 | 0.0605 | 2317 |
| 2001 | | | | | | | | | | | | | | | | | | |
| Phage- | L | E | K | — | — | I | — | S | — | — | — | E | — | M | 0.0601 | 1.8515 | 0.0942 | 2318 |
| 2002 | | | | | | | | | | | | | | | | | | |
| Phage- | F | E | K | W | — | V | — | I | — | — | — | R | D | S | 0.1312 | 1.8508 | 0.1606 | 2319 |
| 2003 | | | | | | | | | | | | | | | | | | |
| Phage- | — | D | N | W | — | R | — | L | — | — | — | H | — | — | 0.0691 | 1.8492 | 0.0907 | 2320 |
| 2004 | | | | | | | | | | | | | | | | | | |
| Phage- | V | T | K | W | — | I | — | E | — | — | — | E | Y | I | 0.0526 | 1.8487 | 0.0658 | 2321 |
| 2005 | | | | | | | | | | | | | | | | | | |
| Phage- | L | P | H | W | — | T | — | L | — | — | — | E | — | — | 0.0582 | 1.8483 | 0.0658 | 2322 |
| 2006 | | | | | | | | | | | | | | | | | | |
| Phage- | Q | D | M | W | — | I | — | P | — | — | — | W | A | Q | 0.0732 | 1.8464 | 0.1257 | 2323 |
| 2007 | | | | | | | | | | | | | | | | | | |
| Phage- | H | P | K | W | — | T | — | A | — | — | — | V | M | L | 0.0606 | 1.8451 | 0.0691 | 2324 |
| 2008 | | | | | | | | | | | | | | | | | | |
| Phage- | E | F | — | K | — | V | — | A | — | — | — | E | W | T | 0.059 | 1.8417 | 0.0768 | 2325 |
| 2009 | | | | | | | | | | | | | | | | | | |
| Phage- | L | Q | K | — | — | I | — | E | — | — | — | L | N | M | 0.0535 | 1.8416 | 0.0655 | 2326 |
| 2010 | | | | | | | | | | | | | | | | | | |
| Phage- | D | T | H | M | — | R | — | L | — | — | — | T | — | L | 0.07 | 1.841 | 0.0778 | 2327 |
| 2011 | | | | | | | | | | | | | | | | | | |
| Phage- | G | E | K | W | — | I | — | P | — | — | — | I | T | N | 0.0747 | 1.8392 | 0.0697 | 2328 |
| 2012 | | | | | | | | | | | | | | | | | | |
| Phage- | L | E | K | W | — | I | — | A | — | — | — | E | V | — | 0.072 | 1.8391 | 0.0967 | 2329 |
| 2013 | | | | | | | | | | | | | | | | | | |
| Phage- | P | E | R | W | — | I | — | V | — | — | — | E | S | G | 0.07 | 1.8312 | 0.0731 | 2330 |
| 2014 | | | | | | | | | | | | | | | | | | |
| Phage- | V | V | Q | — | — | S | — | V | — | — | — | E | — | — | 0.0734 | 1.8298 | 0.086 | 2331 |
| 2015 | | | | | | | | | | | | | | | | | | |
| Phage- | P | I | R | — | — | I | — | E | — | — | — | E | M | V | 0.0577 | 1.8274 | 0.0575 | 2332 |
| 2016 | | | | | | | | | | | | | | | | | | |
| Phage- | M | V | K | W | — | F | — | E | — | — | — | W | T | D | 0.083 | 1.8274 | 0.1357 | 2333 |
| 2017 | | | | | | | | | | | | | | | | | | |
| Phage- | L | D | K | W | — | T | — | S | — | — | — | Y | — | M | 0.0792 | 1.8253 | 0.1065 | 2334 |
| 2018 | | | | | | | | | | | | | | | | | | |
| Phage- | — | V | K | W | — | T | — | P | — | — | — | T | I | Y | 0.1573 | 1.8253 | 0.1533 | 2335 |
| 2019 | | | | | | | | | | | | | | | | | | |
| Phage- | V | I | K | — | — | S | — | P | — | — | — | I | — | L | 0.0675 | 1.8243 | 0.0946 | 2336 |
| 2020 | | | | | | | | | | | | | | | | | | |
| Phage- | T | E | I | W | — | I | — | P | — | — | — | D | — | Q | 0.0642 | 1.8237 | 0.0686 | 2337 |
| 2021 | | | | | | | | | | | | | | | | | | |
| Phage- | Q | L | N | W | — | I | — | E | — | — | — | Q | — | L | 0.0621 | 1.8234 | 0.1244 | 2338 |
| 2022 | | | | | | | | | | | | | | | | | | |
| Phage- | A | L | M | W | — | I | — | Q | — | | — | Q | D | Q | 0.0839 | 1.8231 | 0.1018 | 2339 |
| 2023 | | | | | | | | | | | | | | | | | | |
| Phage- | V | Q | K | W | — | I | — | A | — | — | — | D | Y | V | 0.077 | 1.8177 | 0.0734 | 2340 |
| 2024 | | | | | | | | | | | | | | | | | | |
| Phage- | E | L | K | R | — | T | — | S | — | — | — | E | — | I | 0.0545 | 1.8176 | 0.059 | 2341 |
| 2025 | | | | | | | | | | | | | | | | | | |
| Phage- | L | E | K | W | — | T | — | L | — | — | — | V | — | I | 0.0695 | 1.8162 | 0.078 | 2342 |
| 2026 | | | | | | | | | | | | | | | | | | |
| Phage- | E | L | Q | R | — | T | — | W | — | — | — | F | D | V | 0.0718 | 1.8147 | 0.0689 | 2343 |
| 2027 | | | | | | | | | | | | | | | | | | |
| Phage- | — | I | K | W | — | I | — | E | — | — | — | Y | M | H | 0.075 | 1.8139 | 0.1042 | 2344 |
| 2028 | | | | | | | | | | | | | | | | | | |
| Phage- | E | G | K | W | — | I | — | P | — | — | — | E | R | N | 0.0562 | 1.8131 | 0.0762 | 2345 |
| 2029 | | | | | | | | | | | | | | | | | | |
| Phage- | F | E | K | W | — | I | — | S | — | — | — | E | N | A | 0.0923 | 1.809 | 0.0725 | 2346 |
| 2030 | | | | | | | | | | | | | | | | | | |
| Phage- | V | E | K | W | — | I | — | S | — | — | — | — | E | V | 0.0608 | 1.8084 | 0.086 | 2347 |
| 2031 | | | | | | | | | | | | | | | | | | |
| Phage- | D | I | K | W | — | I | — | P | — | — | — | Q | V | E | 0.0597 | 1.8075 | 0.0691 | 2348 |
| 2032 | | | | | | | | | | | | | | | | | | |
| Phage- | W | P | M | — | — | I | — | E | — | — | — | D | — | L | 0.0797 | 1.804 | 0.0977 | 2349 |
| 2033 | | | | | | | | | | | | | | | | | | |
| Phage- | — | V | K | W | — | T | — | E | — | — | — | V | — | — | 0.0672 | 1.8036 | 0.1541 | 2350 |
| 2034 | | | | | | | | | | | | | | | | | | |
| Phage- | S | L | N | W | — | I | — | E | — | — | — | E | — | — | 0.0697 | 1.8034 | 0.0793 | 2351 |
| 2035 | | | | | | | | | | | | | | | | | | |
| Phage- | E | — | K | W | — | I | — | A | — | — | — | E | D | A | 0.0576 | 1.7994 | 0.0685 | 2352 |
| 2036 | | | | | | | | | | | | | | | | | | |
| Phage- | A | S | P | M | — | I | — | E | — | — | — | S | — | V | 0.0644 | 1.7986 | 0.0687 | 2353 |
| 2037 | | | | | | | | | | | | | | | | | | |
| Phage- | M | E | R | W | — | K | — | A | — | — | — | — | D | M | 0.0634 | 1.7975 | 0.0809 | 2354 |
| 2038 | | | | | | | | | | | | | | | | | | |
| Phage- | D | L | P | W | — | I | — | A | — | — | — | M | — | L | 0.0521 | 1.7969 | 0.0665 | 2355 |
| 2039 | | | | | | | | | | | | | | | | | | |
| Phage- | V | H | K | W | — | I | — | S | — | — | — | E | A | P | 0.0658 | 1.7946 | 0.0914 | 2356 |
| 2040 | | | | | | | | | | | | | | | | | | |
| Phage- | Q | M | K | W | — | T | — | E | — | — | — | V | — | M | 0.056 | 1.7922 | 0.0767 | 2357 |
| 2041 | | | | | | | | | | | | | | | | | | |
| Phage- | M | D | H | W | — | T | — | P | — | — | — | E | — | W | 0.0587 | 1.7921 | 0.0642 | 2358 |
| 2042 | | | | | | | | | | | | | | | | | | |
| Phage- | V | P | K | W | — | T | — | S | — | — | — | E | S | T | 0.0607 | 1.7908 | 0.0787 | 2359 |
| 2043 | | | | | | | | | | | | | | | | | | |
| Phage- | E | V | K | W | — | T | — | E | — | — | — | — | F | L | 0.0614 | 1.7868 | 0.0578 | 2360 |
| 2044 | | | | | | | | | | | | | | | | | | |
| Phage- | — | D | K | W | — | I | — | M | — | — | — | E | M | S | 0.0612 | 1.7863 | 0.0708 | 2361 |
| 2045 | | | | | | | | | | | | | | | | | | |
| Phage- | E | V | H | R | — | I | — | A | — | — | — | T | T | P | 0.0701 | 1.7851 | 0.0934 | 2362 |
| 2046 | | | | | | | | | | | | | | | | | | |
| Phage- | V | D | H | W | — | T | — | A | — | — | — | D | — | — | 0.0677 | 1.7838 | 0.1061 | 2363 |
| 2047 | | | | | | | | | | | | | | | | | | |
| Phage- | V | — | K | W | — | T | — | P | — | — | — | — | W | L | 0.1343 | 1.783 | 0.1974 | 2364 |
| 2048 | | | | | | | | | | | | | | | | | | |
| Phage- | V | M | P | W | — | I | — | Q | — | — | — | V | — | E | 0.066 | 1.7811 | 0.0873 | 2365 |
| 2049 | | | | | | | | | | | | | | | | | | |
| Phage- | E | — | I | W | — | V | — | K | — | — | — | E | — | M | 0.0607 | 1.7808 | 0.0786 | 2366 |
| 2050 | | | | | | | | | | | | | | | | | | |
| Phage- | V | P | K | W | — | T | — | A | — | — | — | N | W | N | 0.0648 | 1.7778 | 0.12 | 2367 |
| 2051 | | | | | | | | | | | | | | | | | | |
| Phage- | F | P | Q | W | — | I | — | I | — | — | — | E | H | T | 0.0616 | 1.7773 | 0.065 | 2368 |
| 2052 | | | | | | | | | | | | | | | | | | |
| Phage- | V | V | H | W | — | I | — | E | — | — | — | E | R | I | 0.0693 | 1.777 | 0.098 | 2369 |
| 2053 | | | | | | | | | | | | | | | | | | |
| Phage- | V | D | K | W | — | I | — | Q | — | — | — | D | D | Q | 0.0608 | 1.7749 | 0.1156 | 2370 |
| 2054 | | | | | | | | | | | | | | | | | | |
| Phage- | A | P | K | — | — | I | — | — | — | — | — | V | S | M | 0.1319 | 1.7748 | 0.0746 | 2371 |
| 2055 | | | | | | | | | | | | | | | | | | |
| Phage- | W | L | N | W | — | T | — | E | — | — | — | N | — | — | 0.0959 | 1.7744 | 0.1468 | 2372 |
| 2056 | | | | | | | | | | | | | | | | | | |
| Phage- | V | E | K | W | — | T | — | P | — | — | — | S | L | — | 0.0569 | 1.7736 | 0.1 | 2373 |
| 2057 | | | | | | | | | | | | | | | | | | |
| Phage- | A | W | K | S | — | I | — | S | — | — | — | E | V | Q | 0.0701 | 1.773 | 0.0942 | 2374 |
| 2058 | | | | | | | | | | | | | | | | | | |
| Phage- | D | T | M | R | — | V | — | E | — | — | — | T | — | — | 0.06 | 1.7719 | 0.066 | 2375 |
| 2059 | | | | | | | | | | | | | | | | | | |
| Phage- | E | H | I | W | — | K | — | D | — | — | — | S | — | — | 0.0638 | 1.7708 | 0.0871 | 2376 |
| 2060 | | | | | | | | | | | | | | | | | | |
| Phage- | E | M | K | W | — | I | — | A | — | — | — | Q | S | H | 0.0609 | 1.7706 | 0.0858 | 2377 |
| 2061 | | | | | | | | | | | | | | | | | | |
| Phage- | E | G | K | W | — | T | — | I | — | — | — | Q | — | — | 0.068 | 1.7706 | 0.1427 | 2378 |
| 2062 | | | | | | | | | | | | | | | | | | |
| Phage- | P | I | K | — | — | T | — | S | — | — | — | I | — | — | 0.0727 | 1.7683 | 0.1075 | 2379 |
| 2063 | | | | | | | | | | | | | | | | | | |
| Phage- | Y | L | H | S | — | T | — | A | — | — | — | T | — | — | 0.0629 | 1.7677 | 0.0668 | 2380 |
| 2064 | | | | | | | | | | | | | | | | | | |
| Phage- | A | — | K | M | — | V | — | W | — | — | — | E | V | M | 0.0579 | 1.7661 | 0.0753 | 2381 |
| 2065 | | | | | | | | | | | | | | | | | | |
| Phage- | V | S | I | M | — | K | — | M | — | — | — | S | — | T | 0.075 | 1.7657 | 0.072 | 2382 |
| 2066 | | | | | | | | | | | | | | | | | | |
| Phage- | P | V | K | W | — | I | — | S | — | — | — | S | V | D | 0.0626 | 1.7647 | 0.0738 | 2383 |
| 2067 | | | | | | | | | | | | | | | | | | |
| Phage- | S | E | K | W | — | I | — | D | — | — | — | Q | V | W | 0.059 | 1.7643 | 0.0929 | 2384 |
| 2068 | | | | | | | | | | | | | | | | | | |
| Phage- | Y | D | H | W | — | T | — | M | — | — | — | H | — | M | 0.0777 | 1.7638 | 0.0688 | 2385 |
| 2069 | | | | | | | | | | | | | | | | | | |
| Phage- | F | E | M | W | — | T | — | S | — | — | — | D | — | — | 0.0605 | 1.7625 | 0.0932 | 2386 |
| 2070 | | | | | | | | | | | | | | | | | | |
| Phage- | — | G | P | W | — | I | — | P | — | — | — | D | H | R | 0.0868 | 1.7624 | 0.0893 | 2387 |
| 2071 | | | | | | | | | | | | | | | | | | |
| Phage- | Q | G | N | W | — | K | — | A | — | — | — | S | — | — | 0.0724 | 1.7622 | 0.0983 | 2388 |
| 2072 | | | | | | | | | | | | | | | | | | |
| Phage- | V | — | K | M | — | I | — | S | — | — | — | — | E | Q | 0.0533 | 1.7605 | 0.0548 | 2389 |
| 2073 | | | | | | | | | | | | | | | | | | |
| Phage- | V | G | N | W | — | K | — | A | — | — | — | E | M | M | 0.0733 | 1.7601 | 0.0681 | 2390 |
| 2074 | | | | | | | | | | | | | | | | | | |
| Phage- | A | D | K | W | — | T | — | T | — | — | — | Q | W | L | 0.0634 | 1.7594 | 0.0745 | 2391 |
| 2075 | | | | | | | | | | | | | | | | | | |
| Phage- | A | D | K | W | — | I | — | E | — | — | — | E | M | N | 0.0701 | 1.7566 | 0.0844 | 2392 |
| 2076 | | | | | | | | | | | | | | | | | | |
| Phage- | A | E | K | — | — | I | — | P | — | — | — | M | L | S | 0.0813 | 1.7556 | 0.101 | 2393 |
| 2077 | | | | | | | | | | | | | | | | | | |
| Phage- | — | E | K | W | — | I | — | E | — | — | — | E | Y | T | 0.0589 | 1.7539 | 0.0826 | 2394 |
| 2078 | | | | | | | | | | | | | | | | | | |
| Phage- | D | W | R | — | — | I | — | E | — | — | — | E | R | L | 0.079 | 1.7529 | 0.0875 | 2395 |
| 2079 | | | | | | | | | | | | | | | | | | |
| Phage- | W | D | N | W | — | T | — | L | — | — | — | Q | — | — | 0.063 | 1.7523 | 0.0794 | 2396 |
| 2080 | | | | | | | | | | | | | | | | | | |
| Phage- | Q | V | N | W | — | I | — | P | — | — | — | — | — | L | 0.0662 | 1.7498 | 0.0898 | 2397 |
| 2081 | | | | | | | | | | | | | | | | | | |
| Phage- | P | E | K | — | — | I | — | D | — | — | — | H | W | D | 0.0913 | 1.7484 | 0.0951 | 2398 |
| 2082 | | | | | | | | | | | | | | | | | | |
| Phage- | W | S | N | W | — | T | — | R | — | V | — | K | T | I | 0.0712 | 1.748 | 0.0752 | 2399 |
| 2083 | | | | | | | | | | | | | | | | | | |
| Phage- | S | E | H | W | — | V | — | S | — | — | — | Y | — | — | 0.0615 | 1.7473 | 0.0957 | 2400 |
| 2084 | | | | | | | | | | | | | | | | | | |
| Phage- | E | S | K | W | — | T | — | P | — | — | — | M | T | D | 0.0672 | 1.747 | 0.0945 | 2401 |
| 2085 | | | | | | | | | | | | | | | | | | |
| Phage- | V | H | Q | M | — | — | — | W | — | — | — | W | E | T | 0.0788 | 1.7462 | 0.0749 | 2402 |
| 2086 | | | | | | | | | | | | | | | | | | |
| Phage- | F | E | K | W | — | I | — | S | — | — | — | S | — | P | 0.0828 | 1.7456 | 0.097 | 2403 |
| 2087 | | | | | | | | | | | | | | | | | | |
| Phage- | V | E | K | W | — | I | — | Q | — | — | — | — | E | M | 0.059 | 1.7432 | 0.0696 | 2404 |
| 2088 | | | | | | | | | | | | | | | | | | |
| Phage- | V | E | K | W | — | I | — | T | — | — | — | Q | A | M | 0.0679 | 1.7416 | 0.0844 | 2405 |
| 2089 | | | | | | | | | | | | | | | | | | |
| Phage- | — | V | K | W | — | V | — | E | — | — | — | E | — | T | 0.0693 | 1.741 | 0.0687 | 2406 |
| 2090 | | | | | | | | | | | | | | | | | | |
| Phage- | E | T | R | W | — | V | — | E | — | — | — | Y | — | I | 0.0583 | 1.7394 | 0.0943 | 2407 |
| 2091 | | | | | | | | | | | | | | | | | | |
| Phage- | F | E | K | W | — | I | — | A | — | — | — | — | A | V | 0.0728 | 1.7389 | 0.1119 | 2408 |
| 2092 | | | | | | | | | | | | | | | | | | |
| Phage- | V | E | T | W | — | T | — | R | — | — | — | E | — | — | 0.0659 | 1.737 | 0.0881 | 2409 |
| 2093 | | | | | | | | | | | | | | | | | | |
| Phage- | V | V | H | W | — | I | — | S | — | — | — | T | E | N | 0.0876 | 1.7367 | 0.0941 | 2410 |
| 2094 | | | | | | | | | | | | | | | | | | |
| Phage- | W | G | K | — | — | I | — | E | — | — | — | T | T | Q | 0.0819 | 1.7364 | 0.0687 | 2411 |
| 2095 | | | | | | | | | | | | | | | | | | |
| Phage- | P | D | H | W | — | I | — | S | — | — | — | M | S | Q | 0.093 | 1.735 | 0.0974 | 2412 |
| 2096 | | | | | | | | | | | | | | | | | | |
| Phage- | Q | G | I | M | — | I | — | E | — | — | — | S | A | I | 0.0513 | 1.7333 | 0.0664 | 2413 |
| 2097 | | | | | | | | | | | | | | | | | | |
| Phage- | — | P | K | — | — | K | — | S | — | — | — | L | F | Y | 0.1284 | 1.7332 | 0.1852 | 2414 |
| 2098 | | | | | | | | | | | | | | | | | | |
| Phage- | L | G | N | W | — | I | — | E | — | — | — | Y | — | M | 0.0693 | 1.7327 | 0.078 | 2415 |
| 2099 | | | | | | | | | | | | | | | | | | |
| Phage- | — | N | M | W | — | I | — | A | — | — | — | Q | N | T | 0.0905 | 1.7323 | 0.1275 | 2416 |
| 2100 | | | | | | | | | | | | | | | | | | |
| Phage- | E | — | K | W | — | I | — | A | — | — | — | S | — | V | 0.0515 | 1.7323 | 0.0654 | 2417 |
| 2101 | | | | | | | | | | | | | | | | | | |
| Phage- | M | E | R | W | — | I | — | E | — | — | — | — | T | T | 0.0563 | 1.7322 | 0.0637 | 2418 |
| 2102 | | | | | | | | | | | | | | | | | | |
| Phage- | S | E | N | W | — | I | — | S | — | — | — | Q | — | — | 0.0784 | 1.7306 | 0.0813 | 2419 |
| 2103 | | | | | | | | | | | | | | | | | | |
| Phage- | G | V | N | W | — | I | — | D | — | — | — | V | — | M | 0.0616 | 1.7294 | 0.0692 | 2420 |
| 2104 | | | | | | | | | | | | | | | | | | |
| Phage- | E | W | K | W | — | T | — | S | — | — | — | E | D | A | 0.0622 | 1.7291 | 0.0652 | 2421 |
| 2105 | | | | | | | | | | | | | | | | | | |
| Phage- | Q | E | K | W | — | I | — | M | — | — | — | E | S | L | 0.078 | 1.7282 | 0.0796 | 2422 |
| 2106 | | | | | | | | | | | | | | | | | | |
| Phage- | V | H | N | W | — | I | — | S | — | — | — | E | — | L | 0.0554 | 1.7266 | 0.0717 | 2423 |
| 2107 | | | | | | | | | | | | | | | | | | |
| Phage- | S | — | M | — | — | K | — | Q | — | — | — | D | — | — | 0.0545 | 1.7237 | 0.0821 | 2424 |
| 2108 | | | | | | | | | | | | | | | | | | |
| Phage- | L | E | N | W | — | K | — | A | — | — | — | R | — | S | 0.0657 | 1.7217 | 0.0678 | 2425 |
| 2109 | | | | | | | | | | | | | | | | | | |
| Phage- | F | E | K | W | — | I | — | P | — | — | — | — | V | H | 0.0776 | 1.7216 | 0.1382 | 2426 |
| 2110 | | | | | | | | | | | | | | | | | | |
| Phage- | V | V | K | W | — | S | — | P | — | — | — | D | F | L | 0.064 | 1.7212 | 0.0713 | 2427 |
| 2111 | | | | | | | | | | | | | | | | | | |
| Phage- | V | D | P | — | — | R | — | A | — | — | — | F | — | M | 0.0586 | 1.7211 | 0.0801 | 2428 |
| 2112 | | | | | | | | | | | | | | | | | | |
| Phage- | M | N | T | W | — | K | — | A | — | — | — | E | — | L | 0.0628 | 1.7208 | 0.127 | 2429 |
| 2113 | | | | | | | | | | | | | | | | | | |
| Phage- | S | E | R | W | — | I | — | E | — | — | — | S | M | P | 0.0527 | 1.7208 | 0.0625 | 2430 |
| 2114 | | | | | | | | | | | | | | | | | | |
| Phage- | G | E | I | W | — | I | — | R | — | — | — | E | S | — | 0.0566 | 1.7201 | 0.0672 | 2431 |
| 2115 | | | | | | | | | | | | | | | | | | |
| Phage- | W | E | N | W | — | T | — | S | — | — | — | S | V | L | 0.0887 | 1.7195 | 0.1179 | 2432 |
| 2116 | | | | | | | | | | | | | | | | | | |
| Phage- | E | I | K | W | — | V | — | Q | — | — | — | E | N | Q | 0.071 | 1.7165 | 0.0643 | 2433 |
| 2117 | | | | | | | | | | | | | | | | | | |
| Phage- | E | M | I | W | — | R | — | A | — | — | — | E | — | I | 0.069 | 1.7157 | 0.0702 | 2434 |
| 2118 | | | | | | | | | | | | | | | | | | |
| Phage- | Y | — | K | W | — | I | — | E | — | — | — | — | V | S | 0.0791 | 1.7154 | 0.12 | 2435 |
| 2119 | | | | | | | | | | | | | | | | | | |
| Phage- | E | I | H | — | — | K | — | P | — | — | — | D | W | L | 0.0666 | 1.715 | 0.1091 | 2436 |
| 2120 | | | | | | | | | | | | | | | | | | |
| Phage- | A | E | K | W | — | I | — | P | — | — | — | E | W | V | 0.0691 | 1.7145 | 0.1046 | 2437 |
| 2121 | | | | | | | | | | | | | | | | | | |
| Phage- | V | T | A | W | — | I | — | E | — | — | — | V | — | V | 0.0736 | 1.714 | 0.0929 | 2438 |
| 2122 | | | | | | | | | | | | | | | | | | |
| Phage- | E | I | K | W | — | R | — | G | — | — | — | K | V | R | 0.0581 | 1.7116 | 0.0583 | 2439 |
| 2123 | | | | | | | | | | | | | | | | | | |
| Phage- | — | L | K | A | — | I | — | D | — | — | — | E | — | S | 0.0658 | 1.7111 | 0.0604 | 2440 |
| 2124 | | | | | | | | | | | | | | | | | | |
| Phage- | E | F | K | W | — | T | — | P | — | — | — | Q | W | D | 0.0644 | 1.7103 | 0.1011 | 2441 |
| 2125 | | | | | | | | | | | | | | | | | | |
| Phage- | S | D | K | W | — | I | — | P | — | — | — | E | V | A | 0.0599 | 1.7096 | 0.0724 | 2442 |
| 2126 | | | | | | | | | | | | | | | | | | |
| Phage- | W | D | K | W | — | I | — | E | — | — | — | V | T | Q | 0.0616 | 1.7083 | 0.1 | 2443 |
| 2127 | | | | | | | | | | | | | | | | | | |
| Phage- | D | G | L | — | — | R | — | L | — | — | — | Q | F | L | 0.0708 | 1.7082 | 0.0771 | 2444 |
| 2128 | | | | | | | | | | | | | | | | | | |
| Phage- | P | L | P | W | — | I | — | V | — | — | — | S | M | I | 0.106 | 1.7067 | 0.1609 | 2445 |
| 2129 | | | | | | | | | | | | | | | | | | |
| Phage- | L | V | K | W | — | T | — | P | — | — | — | E | S | Q | 0.0645 | 1.7064 | 0.0701 | 2446 |
| 2130 | | | | | | | | | | | | | | | | | | |
| Phage- | L | M | K | W | — | I | — | D | — | — | — | S | H | V | 0.0755 | 1.7051 | 0.0763 | 2447 |
| 2131 | | | | | | | | | | | | | | | | | | |
| Phage- | D | — | N | W | — | K | — | T | — | — | — | — | — | — | 0.1202 | 1.7031 | 0.109 | 2448 |
| 2132 | | | | | | | | | | | | | | | | | | |
| Phage- | F | E | M | — | — | T | — | P | — | — | — | V | — | L | 0.0626 | 1.7024 | 0.0639 | 2449 |
| 2133 | | | | | | | | | | | | | | | | | | |
| Phage- | E | G | K | W | — | I | — | E | — | — | — | S | A | S | 0.0768 | 1.7022 | 0.0777 | 2450 |
| 2134 | | | | | | | | | | | | | | | | | | |
| Phage- | T | E | R | W | — | V | — | A | — | — | — | T | L | V | 0.0628 | 1.7021 | 0.0741 | 2451 |
| 2135 | | | | | | | | | | | | | | | | | | |
| Phage- | V | Y | K | W | — | T | — | P | — | — | — | T | Q | M | 0.0722 | 1.6991 | 0.0779 | 2452 |
| 2136 | | | | | | | | | | | | | | | | | | |
| Phage- | V | L | K | W | — | I | — | T | — | — | — | E | T | Q | 0.0716 | 1.697 | 0.0908 | 2453 |
| 2137 | | | | | | | | | | | | | | | | | | |
| Phage- | V | Q | K | W | — | I | — | E | — | — | — | — | V | A | 0.0511 | 1.6967 | 0.0584 | 2454 |
| 2138 | | | | | | | | | | | | | | | | | | |
| Phage- | L | E | K | W | — | T | — | S | — | — | — | I | — | I | 0.0675 | 1.6954 | 0.0773 | 2455 |
| 2139 | | | | | | | | | | | | | | | | | | |
| Phage- | E | H | K | W | — | I | — | S | — | — | — | — | D | M | 0.079 | 1.6944 | 0.1078 | 2456 |
| 2140 | | | | | | | | | | | | | | | | | | |
| Phage- | E | M | K | — | — | R | — | A | — | — | — | E | Y | M | 0.0636 | 1.6928 | 0.0727 | 2457 |
| 2141 | | | | | | | | | | | | | | | | | | |
| Phage- | — | P | K | W | — | T | — | V | — | — | — | E | — | M | 0.0624 | 1.6925 | 0.0647 | 2458 |
| 2142 | | | | | | | | | | | | | | | | | | |
| Phage- | V | W | H | W | — | T | — | P | — | — | — | N | A | E | 0.1156 | 1.6924 | 0.1415 | 2459 |
| 2143 | | | | | | | | | | | | | | | | | | |
| Phage- | A | E | K | W | — | I | — | — | — | — | — | E | A | G | 0.0599 | 1.6921 | 0.0588 | 2460 |
| 2144 | | | | | | | | | | | | | | | | | | |
| Phage- | M | V | N | W | — | T | — | P | — | — | — | W | — | — | 0.161 | 1.6911 | 0.194 | 2461 |
| 2145 | | | | | | | | | | | | | | | | | | |
| Phage- | S | — | P | W | — | I | — | E | — | — | — | F | S | L | 0.0697 | 1.6911 | 0.1013 | 2462 |
| 2146 | | | | | | | | | | | | | | | | | | |
| Phage- | V | E | K | W | — | I | — | Q | — | — | — | V | S | Q | 0.0657 | 1.6881 | 0.0673 | 2463 |
| 2147 | | | | | | | | | | | | | | | | | | |
| Phage- | — | M | K | — | — | M | — | P | — | — | — | — | A | M | 0.0762 | 1.6825 | 0.0907 | 2464 |
| 2148 | | | | | | | | | | | | | | | | | | |
| Phage- | — | T | N | W | — | K | — | A | — | — | — | E | V | S | 0.0627 | 1.6803 | 0.1135 | 2465 |
| 2149 | | | | | | | | | | | | | | | | | | |
| Phage- | V | H | I | W | — | I | — | Q | — | — | — | M | D | R | 0.1186 | 1.6791 | 0.2024 | 2466 |
| 2150 | | | | | | | | | | | | | | | | | | |
| Phage- | Y | E | I | R | — | I | — | P | — | — | — | — | F | D | 0.0649 | 1.6753 | 0.1167 | 2467 |
| 2151 | | | | | | | | | | | | | | | | | | |
| Phage- | — | E | K | W | — | T | — | A | — | — | — | Y | — | I | 0.0569 | 1.675 | 0.077 | 2468 |
| 2152 | | | | | | | | | | | | | | | | | | |
| Phage- | E | H | K | W | — | I | — | D | — | — | — | M | V | A | 0.0663 | 1.6699 | 0.0728 | 2469 |
| 2153 | | | | | | | | | | | | | | | | | | |
| Phage- | L | D | K | W | — | T | — | E | — | — | — | W | — | I | 0.0554 | 1.6696 | 0.0536 | 2470 |
| 2154 | | | | | | | | | | | | | | | | | | |
| Phage- | E | G | R | W | — | I | — | S | — | — | — | V | — | V | 0.0645 | 1.6695 | 0.0956 | 2471 |
| 2155 | | | | | | | | | | | | | | | | | | |
| Phage- | V | S | K | W | — | T | — | E | — | — | — | T | — | — | 0.0559 | 1.6685 | 0.0826 | 2472 |
| 2156 | | | | | | | | | | | | | | | | | | |
| Phage- | D | V | I | R | — | T | — | D | — | — | — | T | — | — | 0.0569 | 1.6642 | 0.064 | 2473 |
| 2157 | | | | | | | | | | | | | | | | | | |
| Phage- | F | E | R | W | — | I | — | A | — | — | — | Q | S | D | 0.0597 | 1.663 | 0.066 | 2474 |
| 2158 | | | | | | | | | | | | | | | | | | |
| Phage- | E | T | K | W | — | F | — | P | — | — | — | — | — | S | 0.0588 | 1.6625 | 0.3374 | 2475 |
| 2159 | | | | | | | | | | | | | | | | | | |
| Phage- | A | M | H | W | — | I | — | K | — | — | — | E | T | S | 0.0999 | 1.659 | 0.141 | 2476 |
| 2160 | | | | | | | | | | | | | | | | | | |
| Phage- | E | M | K | W | — | I | — | D | — | — | — | — | A | G | 0.0724 | 1.6581 | 0.0716 | 2477 |
| 2161 | | | | | | | | | | | | | | | | | | |
| Phage- | E | V | K | — | — | R | — | P | — | — | — | E | I | — | 0.0504 | 1.6567 | 0.0633 | 2478 |
| 2162 | | | | | | | | | | | | | | | | | | |
| Phage- | G | S | H | W | — | I | — | P | — | — | — | Q | M | S | 0.056 | 1.6562 | 0.0821 | 2479 |
| 2163 | | | | | | | | | | | | | | | | | | |
| Phage- | D | I | R | W | — | I | — | D | — | — | — | T | E | T | 0.0557 | 1.6547 | 0.0725 | 2480 |
| 2164 | | | | | | | | | | | | | | | | | | |
| Phage- | V | V | P | W | — | I | — | V | — | — | — | N | S | Q | 0.0859 | 1.6492 | 0.0957 | 2481 |
| 2165 | | | | | | | | | | | | | | | | | | |
| Phage- | Y | E | K | W | — | T | — | A | — | — | — | E | Y | Q | 0.0522 | 1.6489 | 0.0648 | 2482 |
| 2166 | | | | | | | | | | | | | | | | | | |
| Phage- | V | Q | K | W | — | V | — | E | — | — | — | G | S | W | 0.0536 | 1.6487 | 0.0837 | 2483 |
| 2167 | | | | | | | | | | | | | | | | | | |
| Phage- | G | E | K | W | — | I | — | E | — | — | — | V | H | L | 0.0646 | 1.6478 | 0.065 | 2484 |
| 2168 | | | | | | | | | | | | | | | | | | |
| Phage- | E | S | M | — | — | R | — | V | — | — | — | V | — | — | 0.0627 | 1.6473 | 0.0711 | 2485 |
| 2169 | | | | | | | | | | | | | | | | | | |
| Phage- | D | E | N | W | — | K | — | L | — | — | — | K | — | — | 0.0684 | 1.6466 | 0.1054 | 2486 |
| 2170 | | | | | | | | | | | | | | | | | | |
| Phage- | G | D | R | — | — | V | — | E | — | — | — | E | M | L | 0.0633 | 1.6465 | 0.0685 | 2487 |
| 2171 | | | | | | | | | | | | | | | | | | |
| Phage- | M | — | M | M | — | I | — | P | — | — | — | D | T | M | 0.0702 | 1.6464 | 0.0889 | 2488 |
| 2172 | | | | | | | | | | | | | | | | | | |
| Phage- | E | N | K | W | — | I | — | T | — | — | — | — | H | E | 0.0521 | 1.6421 | 0.0646 | 2489 |
| 2173 | | | | | | | | | | | | | | | | | | |
| Phage- | Y | V | N | W | — | T | — | E | — | — | — | V | — | — | 0.0586 | 1.6412 | 0.0598 | 2490 |
| 2174 | | | | | | | | | | | | | | | | | | |
| Phage- | V | I | P | W | — | I | — | E | — | — | — | T | A | S | 0.0655 | 1.6392 | 0.0692 | 2491 |
| 2175 | | | | | | | | | | | | | | | | | | |
| Phage- | V | E | H | W | — | T | — | R | — | — | — | R | — | — | 0.0655 | 1.6388 | 0.0605 | 2492 |
| 2176 | | | | | | | | | | | | | | | | | | |
| Phage- | S | L | H | — | — | I | — | Q | — | — | — | G | I | S | 0.0786 | 1.6388 | 0.0806 | 2493 |
| 2177 | | | | | | | | | | | | | | | | | | |
| Phage- | — | T | K | W | — | T | — | A | — | — | — | — | H | L | 0.0884 | 1.6388 | 0.0904 | 2494 |
| 2178 | | | | | | | | | | | | | | | | | | |
| Phage- | E | P | K | — | — | R | — | A | — | — | — | V | — | M | 0.0854 | 1.6379 | 0.0704 | 2495 |
| 2179 | | | | | | | | | | | | | | | | | | |
| Phage- | L | I | K | W | — | T | — | P | — | — | — | E | Q | L | 0.0633 | 1.6377 | 0.0655 | 2496 |
| 2180 | | | | | | | | | | | | | | | | | | |
| Phage- | L | D | K | — | — | I | — | S | — | — | — | W | S | H | 0.0654 | 1.6364 | 0.0713 | 2497 |
| 2181 | | | | | | | | | | | | | | | | | | |
| Phage- | H | E | K | — | — | I | — | W | — | — | — | E | D | H | 0.0704 | 1.6352 | 0.0868 | 2498 |
| 2182 | | | | | | | | | | | | | | | | | | |
| Phage- | V | H | M | R | — | I | — | E | — | — | — | Q | V | — | 0.1637 | 1.6349 | 0.071 | 2499 |
| 2183 | | | | | | | | | | | | | | | | | | |
| Phage- | T | E | K | W | — | I | — | E | — | — | — | — | H | Q | 0.0643 | 1.6332 | 0.0839 | 2500 |
| 2184 | | | | | | | | | | | | | | | | | | |
| Phage- | W | S | K | S | — | T | — | A | — | — | — | E | — | — | 0.0645 | 1.633 | 0.0843 | 2501 |
| 2185 | | | | | | | | | | | | | | | | | | |
| Phage- | E | F | K | W | — | I | — | A | — | — | — | T | A | A | 0.066 | 1.6322 | 0.0998 | 2502 |
| 2186 | | | | | | | | | | | | | | | | | | |
| Phage- | E | I | K | W | — | T | — | A | — | — | — | E | — | — | 0.0623 | 1.6315 | 0.0762 | 2503 |
| 2187 | | | | | | | | | | | | | | | | | | |
| Phage- | L | S | H | W | — | S | — | Q | — | — | — | S | — | — | 0.0689 | 1.6315 | 0.0932 | 2504 |
| 2188 | | | | | | | | | | | | | | | | | | |
| Phage- | W | Y | K | W | — | T | — | A | — | — | — | E | F | T | 0.1438 | 1.6308 | 0.145 | 2505 |
| 2189 | | | | | | | | | | | | | | | | | | |
| Phage- | E | H | H | W | — | I | — | K | — | — | — | N | E | V | 0.0682 | 1.6293 | 0.0711 | 2506 |
| 2190 | | | | | | | | | | | | | | | | | | |
| Phage- | V | D | N | W | — | V | — | R | — | — | — | D | — | — | 0.06 | 1.6289 | 0.0922 | 2507 |
| 2191 | | | | | | | | | | | | | | | | | | |
| Phage- | M | T | P | W | — | K | — | A | — | — | — | Q | — | I | 0.0788 | 1.6288 | 0.0994 | 2508 |
| 2192 | | | | | | | | | | | | | | | | | | |
| Phage- | E | S | H | — | — | I | — | A | — | — | — | D | V | Q | 0.0742 | 1.6277 | 0.0973 | 2509 |
| 2193 | | | | | | | | | | | | | | | | | | |
| Phage- | H | — | M | W | — | I | — | A | — | — | — | E | N | L | 0.0547 | 1.6272 | 0.0809 | 2510 |
| 2194 | | | | | | | | | | | | | | | | | | |
| Phage- | L | Y | K | W | — | M | — | A | — | — | — | E | Q | K | 0.0532 | 1.6238 | 0.0702 | 2511 |
| 2195 | | | | | | | | | | | | | | | | | | |
| Phage- | L | — | D | W | — | R | — | V | — | — | — | E | — | — | 0.1457 | 1.6232 | 0.2873 | 2512 |
| 2196 | | | | | | | | | | | | | | | | | | |
| Phage- | L | E | K | W | — | I | — | S | — | — | — | D | — | M | 0.0664 | 1.6214 | 0.0637 | 2513 |
| 2197 | | | | | | | | | | | | | | | | | | |
| Phage- | D | V | N | W | — | T | — | P | — | — | — | W | — | — | 0.0746 | 1.6213 | 0.0743 | 2514 |
| 2198 | | | | | | | | | | | | | | | | | | |
| Phage- | V | E | K | W | — | I | — | S | — | — | — | Q | E | L | 0.0551 | 1.6198 | 0.0642 | 2515 |
| 2199 | | | | | | | | | | | | | | | | | | |
| Phage- | G | E | I | W | — | I | — | A | — | — | — | N | T | G | 0.058 | 1.6178 | 0.0549 | 2516 |
| 2200 | | | | | | | | | | | | | | | | | | |
| Phage- | E | Y | N | W | — | T | — | R | — | — | — | K | — | — | 0.0537 | 1.6159 | 0.0617 | 2517 |
| 2201 | | | | | | | | | | | | | | | | | | |
| Phage- | H | D | K | W | — | I | — | E | — | — | — | I | S | L | 0.0584 | 1.6151 | 0.075 | 2518 |
| 2202 | | | | | | | | | | | | | | | | | | |
| Phage- | M | E | R | W | — | I | — | E | — | — | — | E | R | T | 0.0786 | 1.6134 | 0.072 | 2519 |
| 2203 | | | | | | | | | | | | | | | | | | |
| Phage- | D | L | K | — | — | K | — | E | — | — | — | N | — | M | 0.0648 | 1.6126 | 0.0815 | 2520 |
| 2204 | | | | | | | | | | | | | | | | | | |
| Phage- | V | D | R | — | — | T | — | E | — | — | — | S | — | — | 0.0624 | 1.6126 | 0.0763 | 2521 |
| 2205 | | | | | | | | | | | | | | | | | | |
| Phage- | L | E | K | W | — | I | — | A | — | — | — | — | V | L | 0.1159 | 1.6113 | 0.078 | 2522 |
| 2206 | | | | | | | | | | | | | | | | | | |
| Phage- | P | T | K | W | — | I | — | E | — | — | — | S | V | I | 0.0543 | 1.6094 | 0.0587 | 2523 |
| 2207 | | | | | | | | | | | | | | | | | | |
| Phage- | A | L | H | W | — | I | — | E | — | — | — | S | S | M | 0.0766 | 1.6077 | 0.1027 | 2524 |
| 2208 | | | | | | | | | | | | | | | | | | |
| Phage- | Q | D | H | W | — | V | — | Q | — | — | — | E | M | L | 0.0587 | 1.6055 | 0.07 | 2525 |
| 2209 | | | | | | | | | | | | | | | | | | |
| Phage- | T | H | N | W | — | T | — | Q | — | — | — | D | — | — | 0.0686 | 1.6043 | 0.0628 | 2526 |
| 2210 | | | | | | | | | | | | | | | | | | |
| Phage- | P | G | K | W | — | I | — | A | — | — | — | S | L | V | 0.07 | 1.601 | 0.1571 | 2527 |
| 2211 | | | | | | | | | | | | | | | | | | |
| Phage- | T | E | K | W | — | I | — | S | — | — | — | E | I | S | 0.0663 | 1.5994 | 0.0837 | 2528 |
| 2212 | | | | | | | | | | | | | | | | | | |
| Phage- | V | I | P | — | — | S | — | P | — | — | — | F | M | S | 0.0568 | 1.5984 | 0.0631 | 2529 |
| 2213 | | | | | | | | | | | | | | | | | | |
| Phage- | P | E | K | W | — | I | — | E | — | — | — | D | A | L | 0.0788 | 1.5953 | 0.0578 | 2530 |
| 2214 | | | | | | | | | | | | | | | | | | |
| Phage- | G | V | K | W | — | T | — | P | — | — | — | E | V | A | 0.0818 | 1.5929 | 0.0672 | 2531 |
| 2215 | | | | | | | | | | | | | | | | | | |
| Phage- | W | — | Q | W | — | T | — | S | — | — | — | E | — | — | 0.0752 | 1.591 | 0.0929 | 2532 |
| 2216 | | | | | | | | | | | | | | | | | | |
| Phage- | E | V | K | W | — | I | — | S | — | — | — | E | S | E | 0.0576 | 1.5894 | 0.0904 | 2533 |
| 2217 | | | | | | | | | | | | | | | | | | |
| Phage- | V | P | K | Q | — | V | — | E | — | — | — | S | — | — | 0.071 | 1.5885 | 0.0779 | 2534 |
| 2218 | | | | | | | | | | | | | | | | | | |
| Phage- | S | E | N | W | — | I | — | A | — | — | — | S | — | — | 0.0867 | 1.5857 | 0.1111 | 2535 |
| 2219 | | | | | | | | | | | | | | | | | | |
| Phage- | E | P | K | W | — | T | — | E | — | — | — | L | N | L | 0.0533 | 1.5844 | 0.0915 | 2536 |
| 2220 | | | | | | | | | | | | | | | | | | |
| Phage- | G | H | Q | W | — | I | — | Q | — | — | — | V | — | L | 0.0576 | 1.5801 | 0.0809 | 2537 |
| 2221 | | | | | | | | | | | | | | | | | | |
| Phage- | T | E | K | W | — | I | — | Q | — | — | — | E | A | T | 0.0945 | 1.5774 | 0.0783 | 2538 |
| 2222 | | | | | | | | | | | | | | | | | | |
| Phage- | F | L | K | W | — | T | — | E | — | — | — | S | D | V | 0.088 | 1.5773 | 0.1279 | 2539 |
| 2223 | | | | | | | | | | | | | | | | | | |
| Phage- | A | I | K | W | — | T | — | P | — | — | — | D | W | H | 0.0734 | 1.5759 | 0.131 | 2540 |
| 2224 | | | | | | | | | | | | | | | | | | |
| Phage- | L | F | K | V | — | T | — | S | — | — | — | S | — | — | 0.0611 | 1.5757 | 0.0813 | 2541 |
| 2225 | | | | | | | | | | | | | | | | | | |
| Phage- | L | — | Y | W | — | T | — | A | — | — | — | E | — | — | 0.0674 | 1.5737 | 0.1122 | 2542 |
| 2226 | | | | | | | | | | | | | | | | | | |
| Phage- | S | E | L | W | — | I | — | P | — | — | — | N | F | L | 0.0985 | 1.5722 | 0.0684 | 2543 |
| 2227 | | | | | | | | | | | | | | | | | | |
| Phage- | L | V | K | W | — | I | — | Q | — | — | — | E | Q | A | 0.0589 | 1.571 | 0.0712 | 2544 |
| 2228 | | | | | | | | | | | | | | | | | | |
| Phage- | L | L | K | W | — | T | — | P | — | — | — | E | L | H | 0.0718 | 1.5707 | 0.0932 | 2545 |
| 2229 | | | | | | | | | | | | | | | | | | |
| Phage- | T | D | M | W | — | I | — | P | — | — | — | E | R | I | 0.0725 | 1.5691 | 0.0846 | 2546 |
| 2230 | | | | | | | | | | | | | | | | | | |
| Phage- | L | S | K | W | — | I | — | E | — | — | — | — | V | M | 0.0694 | 1.5657 | 0.0848 | 2547 |
| 2231 | | | | | | | | | | | | | | | | | | |
| Phage- | Q | L | N | W | — | R | — | A | — | — | — | E | — | — | 0.0619 | 1.5654 | 0.1277 | 2548 |
| 2232 | | | | | | | | | | | | | | | | | | |
| Phage- | E | I | Q | W | — | T | — | S | — | — | — | P | W | L | 0.0648 | 1.5653 | 0.0668 | 2549 |
| 2233 | | | | | | | | | | | | | | | | | | |
| Phage- | M | P | N | W | — | T | — | T | — | — | — | Q | — | — | 0.0505 | 1.5653 | 0.0682 | 2550 |
| 2234 | | | | | | | | | | | | | | | | | | |
| Phage- | F | N | K | W | — | V | — | A | — | — | — | S | D | M | 0.1089 | 1.563 | 0.0946 | 2551 |
| 2235 | | | | | | | | | | | | | | | | | | |
| Phage- | P | R | K | W | — | I | — | A | — | — | — | E | I | I | 0.0741 | 1.5618 | 0.0733 | 2552 |
| 2236 | | | | | | | | | | | | | | | | | | |
| Phage- | V | S | A | W | — | I | — | P | — | — | — | G | T | N | 0.069 | 1.561 | 0.0801 | 2553 |
| 2237 | | | | | | | | | | | | | | | | | | |
| Phage- | D | F | K | W | — | I | — | T | — | — | — | E | V | Y | 0.0613 | 1.5607 | 0.0768 | 2554 |
| 2238 | | | | | | | | | | | | | | | | | | |
| Phage- | H | P | N | W | — | T | — | P | — | — | — | L | — | L | 0.0764 | 1.5601 | 0.0699 | 2555 |
| 2239 | | | | | | | | | | | | | | | | | | |
| Phage- | G | T | M | W | — | I | — | P | — | — | — | S | A | Q | 0.0769 | 1.5599 | 0.0766 | 2556 |
| 2240 | | | | | | | | | | | | | | | | | | |
| Phage- | V | V | K | W | — | I | — | T | — | — | — | E | N | S | 0.0674 | 1.5594 | 0.0889 | 2557 |
| 2241 | | | | | | | | | | | | | | | | | | |
| Phage- | L | M | N | W | — | I | — | P | — | — | — | S | A | Q | 0.0812 | 1.5589 | 0.0907 | 2558 |
| 2242 | | | | | | | | | | | | | | | | | | |
| Phage- | L | M | K | W | — | I | — | Q | — | — | — | Q | L | D | 0.0717 | 1.5582 | 0.2491 | 2559 |
| 2243 | | | | | | | | | | | | | | | | | | |
| Phage- | L | V | K | — | — | I | — | E | — | — | — | S | E | G | 0.0605 | 1.5566 | 0.0711 | 2560 |
| 2244 | | | | | | | | | | | | | | | | | | |
| Phage- | S | E | K | — | — | I | — | E | — | — | — | — | Q | A | 0.0531 | 1.5553 | 0.0686 | 2561 |
| 2245 | | | | | | | | | | | | | | | | | | |
| Phage- | G | E | I | W | — | T | — | K | — | — | — | D | — | — | 0.0694 | 1.5549 | 0.0716 | 2562 |
| 2246 | | | | | | | | | | | | | | | | | | |
| Phage- | N | I | I | R | — | T | — | A | — | — | — | D | — | — | 0.0587 | 1.5528 | 0.0619 | 2563 |
| 2247 | | | | | | | | | | | | | | | | | | |
| Phage- | L | E | N | W | — | K | — | A | — | — | — | W | — | M | 0.0694 | 1.5514 | 0.0811 | 2564 |
| 2248 | | | | | | | | | | | | | | | | | | |
| Phage- | — | I | K | W | — | T | — | E | — | — | — | V | — | — | 0.0584 | 1.5499 | 0.0748 | 2565 |
| 2249 | | | | | | | | | | | | | | | | | | |
| Phage- | — | D | K | W | — | I | — | A | — | — | — | E | S | T | 0.0575 | 1.5492 | 0.1055 | 2566 |
| 2250 | | | | | | | | | | | | | | | | | | |
| Phage- | G | T | N | W | — | T | — | A | — | — | — | — | — | — | 0.0567 | 1.5478 | 0.061 | 2567 |
| 2251 | | | | | | | | | | | | | | | | | | |
| Phage- | A | D | K | W | — | I | — | E | — | — | — | Q | Q | D | 0.0537 | 1.5471 | 0.0665 | 2568 |
| 2252 | | | | | | | | | | | | | | | | | | |
| Phage- | N | E | R | W | — | I | — | S | — | — | — | Q | E | A | 0.0527 | 1.5469 | 0.0574 | 2569 |
| 2253 | | | | | | | | | | | | | | | | | | |
| Phage- | P | — | K | W | — | I | — | M | — | — | — | Q | V | D | 0.0867 | 1.5458 | 0.0779 | 2570 |
| 2254 | | | | | | | | | | | | | | | | | | |
| Phage- | V | E | L | — | — | K | — | P | — | — | — | S | — | L | 0.0711 | 1.5444 | 0.0931 | 2571 |
| 2255 | | | | | | | | | | | | | | | | | | |
| Phage- | P | V | N | W | — | K | — | P | — | — | — | E | M | W | 0.0673 | 1.544 | 0.087 | 2572 |
| 2256 | | | | | | | | | | | | | | | | | | |
| Phage- | M | E | M | W | — | R | — | A | — | — | — | — | H | S | 0.0747 | 1.5437 | 0.0767 | 2573 |
| 2257 | | | | | | | | | | | | | | | | | | |
| Phage- | M | M | K | W | — | T | — | E | — | — | — | — | M | V | 0.0607 | 1.5394 | 0.0753 | 2574 |
| 2258 | | | | | | | | | | | | | | | | | | |
| Phage- | D | M | R | W | — | I | — | A | — | — | — | E | D | R | 0.0524 | 1.5389 | 0.0623 | 2575 |
| 2259 | | | | | | | | | | | | | | | | | | |
| Phage- | V | D | R | W | — | I | — | E | — | — | — | N | E | L | 0.059 | 1.5387 | 0.084 | 2576 |
| 2260 | | | | | | | | | | | | | | | | | | |
| Phage- | T | E | K | W | — | I | — | A | — | — | — | Q | V | S | 0.0888 | 1.5377 | 0.0738 | 2577 |
| 2261 | | | | | | | | | | | | | | | | | | |
| Phage- | P | N | I | W | — | T | — | L | — | — | — | E | — | L | 0.0705 | 1.5369 | 0.0772 | 2578 |
| 2262 | | | | | | | | | | | | | | | | | | |
| Phage- | Y | S | H | — | — | I | — | S | — | — | — | S | W | T | 0.0855 | 1.5364 | 0.1108 | 2579 |
| 2263 | | | | | | | | | | | | | | | | | | |
| Phage- | A | L | K | W | — | I | — | D | — | — | — | S | Q | P | 0.0669 | 1.5356 | 0.0749 | 2580 |
| 2264 | | | | | | | | | | | | | | | | | | |
| Phage- | G | D | R | W | — | I | — | E | — | — | — | G | V | I | 0.0567 | 1.5355 | 0.0664 | 2581 |
| 2265 | | | | | | | | | | | | | | | | | | |
| Phage- | E | M | H | W | — | I | — | A | — | — | — | P | D | G | 0.0546 | 1.5353 | 0.0572 | 2582 |
| 2266 | | | | | | | | | | | | | | | | | | |
| Phage- | — | T | K | — | — | T | — | P | — | — | — | T | M | V | 0.0546 | 1.5325 | 0.0558 | 2583 |
| 2267 | | | | | | | | | | | | | | | | | | |
| Phage- | A | E | N | W | — | R | — | S | — | — | — | E | — | — | 0.0802 | 1.5288 | 0.0717 | 2584 |
| 2268 | | | | | | | | | | | | | | | | | | |
| Phage- | F | — | K | W | — | T | — | Q | — | — | — | S | A | M | 0.1268 | 1.5282 | 0.2251 | 2585 |
| 2269 | | | | | | | | | | | | | | | | | | |
| Phage- | V | V | K | W | — | S | — | P | — | — | — | E | Y | — | 0.0672 | 1.5269 | 0.0886 | 2586 |
| 2270 | | | | | | | | | | | | | | | | | | |
| Phage- | H | Q | N | W | — | I | — | A | — | — | — | H | — | M | 0.0572 | 1.5253 | 0.0592 | 2587 |
| 2271 | | | | | | | | | | | | | | | | | | |
| Phage- | W | D | M | R | — | T | — | I | — | — | — | E | — | — | 0.0572 | 1.525 | 0.0882 | 2588 |
| 2272 | | | | | | | | | | | | | | | | | | |
| Phage- | G | I | N | W | — | I | — | P | — | — | — | Q | S | E | 0.0713 | 1.5248 | 0.1189 | 2589 |
| 2273 | | | | | | | | | | | | | | | | | | |
| Phage- | Y | T | N | W | — | I | — | S | — | — | — | E | — | — | 0.061 | 1.5216 | 0.0789 | 2590 |
| 2274 | | | | | | | | | | | | | | | | | | |
| Phage- | G | E | K | W | — | I | — | S | — | — | — | E | I | T | 0.0597 | 1.5204 | 0.0796 | 2591 |
| 2275 | | | | | | | | | | | | | | | | | | |
| Phage- | L | E | K | W | — | T | — | P | — | — | — | M | — | — | 0.0549 | 1.5198 | 0.0948 | 2592 |
| 2276 | | | | | | | | | | | | | | | | | | |
| Phage- | — | I | K | W | — | K | — | L | — | — | — | E | S | I | 0.0987 | 1.5193 | 0.1037 | 2593 |
| 2277 | | | | | | | | | | | | | | | | | | |
| Phage- | — | E | K | W | — | I | — | Q | — | — | — | S | A | A | 0.0564 | 1.5183 | 0.0739 | 2594 |
| 2278 | | | | | | | | | | | | | | | | | | |
| Phage- | — | P | R | M | — | T | — | A | — | — | — | E | W | S | 0.0674 | 1.5139 | 0.0738 | 2595 |
| 2279 | | | | | | | | | | | | | | | | | | |
| Phage- | F | — | I | — | — | K | — | M | — | — | — | E | H | I | 0.0595 | 1.5129 | 0.0895 | 2596 |
| 2280 | | | | | | | | | | | | | | | | | | |
| Phage- | M | P | K | W | — | I | — | T | — | — | — | E | S | T | 0.0575 | 1.5105 | 0.054 | 2597 |
| 2281 | | | | | | | | | | | | | | | | | | |
| Phage- | A | V | K | — | — | T | — | M | — | — | — | M | — | — | 0.0795 | 1.5103 | 0.0984 | 2598 |
| 2282 | | | | | | | | | | | | | | | | | | |
| Phage- | G | G | K | W | — | K | — | S | — | — | — | F | — | M | 0.067 | 1.5103 | 0.1079 | 2599 |
| 2283 | | | | | | | | | | | | | | | | | | |
| Phage- | M | I | N | W | — | T | — | A | — | — | — | T | — | — | 0.0675 | 1.5101 | 0.1007 | 2600 |
| 2284 | | | | | | | | | | | | | | | | | | |
| Phage- | E | P | K | W | — | I | — | Q | — | — | — | E | R | — | 0.069 | 1.51 | 0.0654 | 2601 |
| 2285 | | | | | | | | | | | | | | | | | | |
| Phage- | A | E | K | W | — | I | — | P | — | — | — | V | N | D | 0.0769 | 1.5074 | 0.0683 | 2602 |
| 2286 | | | | | | | | | | | | | | | | | | |
| Phage- | V | E | K | W | — | T | — | R | — | — | — | E | — | — | 0.066 | 1.5059 | 0.0841 | 2603 |
| 2287 | | | | | | | | | | | | | | | | | | |
| Phage- | H | V | K | W | — | T | — | A | — | — | — | V | S | N | 0.07 | 1.5053 | 0.079 | 2604 |
| 2288 | | | | | | | | | | | | | | | | | | |
| Phage- | E | L | N | W | — | T | — | P | — | — | — | N | P | M | 0.0572 | 1.5034 | 0.0786 | 2605 |
| 2289 | | | | | | | | | | | | | | | | | | |
| Phage- | V | D | K | Q | — | R | — | A | — | — | — | E | I | W | 0.0552 | 1.4998 | 0.0653 | 2606 |
| 2290 | | | | | | | | | | | | | | | | | | |
| Phage- | V | E | K | W | — | I | — | A | — | — | — | Q | Q | S | 0.0763 | 1.4997 | 0.0698 | 2607 |
| 2291 | | | | | | | | | | | | | | | | | | |
| Phage- | N | S | N | W | — | I | — | S | — | — | — | — | D | I | 0.066 | 1.4992 | 0.0741 | 2608 |
| 2292 | | | | | | | | | | | | | | | | | | |
| Phage- | V | E | K | W | — | I | — | P | — | — | — | V | N | S | 0.0648 | 1.499 | 0.0666 | 2609 |
| 2293 | | | | | | | | | | | | | | | | | | |
| Phage- | — | H | M | — | — | S | — | P | — | — | — | V | — | L | 0.0727 | 1.4982 | 0.0564 | 2610 |
| 2294 | | | | | | | | | | | | | | | | | | |
| Phage- | D | V | M | W | — | I | — | P | — | — | — | E | H | R | 0.0787 | 1.4965 | 0.1062 | 2611 |
| 2295 | | | | | | | | | | | | | | | | | | |
| Phage- | N | V | K | W | — | T | — | P | — | — | — | F | L | D | 0.0601 | 1.4963 | 0.0656 | 2612 |
| 2296 | | | | | | | | | | | | | | | | | | |
| Phage- | T | H | K | W | — | I | — | V | — | — | — | E | H | Q | 0.0753 | 1.4957 | 0.0929 | 2613 |
| 2297 | | | | | | | | | | | | | | | | | | |
| Phage- | W | E | N | W | — | T | — | E | — | — | — | N | — | — | 0.0575 | 1.4939 | 0.0767 | 2614 |
| 2298 | | | | | | | | | | | | | | | | | | |
| Phage- | M | D | N | W | — | T | — | P | — | — | — | H | — | — | 0.0635 | 1.4932 | 0.0898 | 2615 |
| 2299 | | | | | | | | | | | | | | | | | | |
| Phage- | W | V | Q | W | — | I | — | P | — | — | — | E | P | S | 0.103 | 1.4928 | 0.1403 | 2616 |
| 2300 | | | | | | | | | | | | | | | | | | |
| Phage- | S | M | K | W | — | T | — | A | — | — | — | E | R | L | 0.1168 | 1.4902 | 0.1151 | 2617 |
| 2301 | | | | | | | | | | | | | | | | | | |
| Phage- | F | — | N | W | — | T | — | S | — | — | — | N | F | T | 0.1515 | 1.4849 | 0.152 | 2618 |
| 2302 | | | | | | | | | | | | | | | | | | |
| Phage- | L | T | K | W | — | I | — | E | — | — | — | — | V | E | 0.0656 | 1.4846 | 0.1018 | 2619 |
| 2303 | | | | | | | | | | | | | | | | | | |
| Phage- | V | Q | N | W | — | I | — | A | — | — | — | E | — | K | 0.0655 | 1.4841 | 0.0686 | 2620 |
| 2304 | | | | | | | | | | | | | | | | | | |
| Phage- | E | — | K | W | — | T | — | P | — | — | — | M | S | M | 0.0601 | 1.4834 | 0.0593 | 2621 |
| 2305 | | | | | | | | | | | | | | | | | | |
| Phage- | Q | N | T | W | — | I | — | P | — | — | — | E | V | — | 0.068 | 1.4804 | 0.0806 | 2622 |
| 2306 | | | | | | | | | | | | | | | | | | |
| Phage- | Y | D | R | — | — | I | — | M | — | — | — | S | S | V | 0.0766 | 1.479 | 0.0715 | 2623 |
| 2307 | | | | | | | | | | | | | | | | | | |
| Phage- | E | W | K | W | — | T | — | P | — | — | — | Q | Y | E | 0.0586 | 1.4772 | 0.0874 | 2624 |
| 2308 | | | | | | | | | | | | | | | | | | |
| Phage- | — | E | K | W | — | I | — | L | — | — | — | E | M | Q | 0.0595 | 1.4765 | 0.0726 | 2625 |
| 2309 | | | | | | | | | | | | | | | | | | |
| Phage- | M | E | I | W | — | K | — | A | — | — | — | E | S | T | 0.0769 | 1.4762 | 0.0714 | 2626 |
| 2310 | | | | | | | | | | | | | | | | | | |
| Phage- | P | E | K | W | — | I | — | D | — | — | — | T | V | Y | 0.0696 | 1.4761 | 0.0815 | 2627 |
| 2311 | | | | | | | | | | | | | | | | | | |
| Phage- | E | F | K | M | — | I | — | Q | — | — | — | Q | H | Q | 0.0566 | 1.4746 | 0.0622 | 2628 |
| 2312 | | | | | | | | | | | | | | | | | | |
| Phage- | L | E | K | W | — | M | — | P | — | — | — | S | — | T | 0.0776 | 1.4726 | 0.0701 | 2629 |
| 2313 | | | | | | | | | | | | | | | | | | |
| Phage- | F | V | N | W | — | I | — | T | — | — | — | P | E | I | 0.1963 | 1.4712 | 0.2332 | 2630 |
| 2314 | | | | | | | | | | | | | | | | | | |
| Phage- | E | — | R | W | — | T | — | P | — | — | — | — | Y | M | 0.0772 | 1.4678 | 0.0724 | 2631 |
| 2315 | | | | | | | | | | | | | | | | | | |
| Phage- | G | M | L | A | — | T | — | A | — | — | — | E | — | — | 0.0856 | 1.4657 | 0.0923 | 2632 |
| 2316 | | | | | | | | | | | | | | | | | | |
| Phage- | M | E | Q | W | — | I | — | Q | — | — | — | S | — | — | 0.0596 | 1.4568 | 0.0911 | 2633 |
| 2317 | | | | | | | | | | | | | | | | | | |
| Phage- | L | E | K | W | — | T | — | V | — | — | — | E | — | V | 0.0627 | 1.456 | 0.0702 | 2634 |
| 2318 | | | | | | | | | | | | | | | | | | |
| Phage- | S | D | N | W | — | K | — | K | — | — | — | E | T | I | 0.0635 | 1.4548 | 0.1155 | 2635 |
| 2319 | | | | | | | | | | | | | | | | | | |
| Phage- | V | S | N | W | — | I | — | R | G | L | — | T | Q | M | 0.0592 | 1.4541 | 0.0665 | 2636 |
| 2320 | | | | | | | | | | | | | | | | | | |
| Phage- | L | D | K | W | — | I | — | P | — | — | — | E | T | V | 0.0504 | 1.454 | 0.0597 | 2637 |
| 2321 | | | | | | | | | | | | | | | | | | |
| Phage- | L | E | K | W | — | I | — | T | — | — | — | E | M | T | 0.064 | 1.4533 | 0.0679 | 2638 |
| 2322 | | | | | | | | | | | | | | | | | | |
| Phage- | W | D | H | W | — | T | — | A | — | — | — | F | N | A | 0.0803 | 1.4512 | 0.1191 | 2639 |
| 2323 | | | | | | | | | | | | | | | | | | |
| Phage- | H | S | H | W | — | I | — | P | — | — | — | T | F | V | 0.1487 | 1.451 | 0.1622 | 2640 |
| 2324 | | | | | | | | | | | | | | | | | | |
| Phage- | V | E | K | — | — | I | — | H | — | — | — | E | A | S | 0.0617 | 1.4507 | 0.0647 | 2641 |
| 2325 | | | | | | | | | | | | | | | | | | |
| Phage- | G | P | T | — | — | I | — | A | — | — | — | S | — | M | 0.0585 | 1.4506 | 0.0802 | 2642 |
| 2326 | | | | | | | | | | | | | | | | | | |
| Phage- | S | V | H | W | — | T | — | P | — | — | — | Q | — | — | 0.0589 | 1.4503 | 0.0771 | 2643 |
| 2327 | | | | | | | | | | | | | | | | | | |
| Phage- | V | D | K | W | — | I | — | A | — | — | — | V | S | A | 0.0636 | 1.4502 | 0.0715 | 2644 |
| 2328 | | | | | | | | | | | | | | | | | | |
| Phage- | P | E | K | W | — | I | — | P | — | — | — | T | N | — | 0.0547 | 1.4495 | 0.065 | 2645 |
| 2329 | | | | | | | | | | | | | | | | | | |
| Phage- | E | V | K | W | — | T | — | S | — | — | — | S | — | L | 0.0537 | 1.4492 | 0.06 | 2646 |
| 2330 | | | | | | | | | | | | | | | | | | |
| Phage- | F | E | H | W | — | T | — | E | — | — | — | L | — | M | 0.0593 | 1.4476 | 0.0623 | 2647 |
| 2331 | | | | | | | | | | | | | | | | | | |
| Phage- | V | V | N | W | — | V | — | E | — | — | — | S | — | — | 0.0514 | 1.447 | 0.0772 | 2648 |
| 2332 | | | | | | | | | | | | | | | | | | |
| Phage- | T | V | K | W | — | I | — | S | — | — | — | Q | Q | S | 0.0947 | 1.4443 | 0.1024 | 2649 |
| 2333 | | | | | | | | | | | | | | | | | | |
| Phage- | E | F | I | R | — | L | — | P | — | — | — | — | — | — | 0.0517 | 1.4439 | 0.0734 | 2650 |
| 2334 | | | | | | | | | | | | | | | | | | |
| Phage- | A | S | N | W | — | I | — | T | — | — | — | E | — | S | 0.0604 | 1.4429 | 0.0671 | 2651 |
| 2335 | | | | | | | | | | | | | | | | | | |
| Phage- | E | P | K | W | — | T | — | A | — | — | — | S | I | V | 0.0654 | 1.4375 | 0.0668 | 2652 |
| 2336 | | | | | | | | | | | | | | | | | | |
| Phage- | M | V | K | — | — | I | — | E | — | — | — | D | R | Q | 0.0645 | 1.4362 | 0.0609 | 2653 |
| 2337 | | | | | | | | | | | | | | | | | | |
| Phage- | W | Y | K | W | — | T | — | A | — | — | — | E | I | S | 0.1377 | 1.4356 | 0.1433 | 2654 |
| 2338 | | | | | | | | | | | | | | | | | | |
| Phage- | E | G | I | S | — | V | — | — | — | — | — | V | — | — | 0.0845 | 1.4356 | 0.1187 | 2655 |
| 2339 | | | | | | | | | | | | | | | | | | |
| Phage- | L | V | H | M | — | K | — | A | — | — | — | T | N | L | 0.105 | 1.4355 | 0.0708 | 2656 |
| 2340 | | | | | | | | | | | | | | | | | | |
| Phage- | G | E | I | W | — | T | — | S | — | — | — | W | — | L | 0.0577 | 1.4349 | 0.0653 | 2657 |
| 2341 | | | | | | | | | | | | | | | | | | |
| Phage- | E | L | M | W | — | I | — | P | — | — | — | E | H | S | 0.0645 | 1.4336 | 0.0888 | 2658 |
| 2342 | | | | | | | | | | | | | | | | | | |
| Phage- | S | D | T | W | — | I | — | A | — | — | — | E | — | R | 0.0602 | 1.4336 | 0.0667 | 2659 |
| 2343 | | | | | | | | | | | | | | | | | | |
| Phage- | L | M | N | W | — | T | — | A | — | — | — | T | — | — | 0.0711 | 1.4335 | 0.0919 | 2660 |
| 2344 | | | | | | | | | | | | | | | | | | |
| Phage- | F | D | M | — | — | N | — | K | — | — | — | E | — | — | 0.0618 | 1.4334 | 0.0643 | 2661 |
| 2345 | | | | | | | | | | | | | | | | | | |
| Phage- | W | D | R | — | — | I | — | P | — | — | — | D | V | M | 0.0695 | 1.4323 | 0.0803 | 2662 |
| 2346 | | | | | | | | | | | | | | | | | | |
| Phage- | S | E | N | W | — | I | — | — | — | — | — | S | — | L | 0.0819 | 1.432 | 0.0725 | 2663 |
| 2347 | | | | | | | | | | | | | | | | | | |
| Phage- | M | E | K | W | — | T | — | S | — | — | — | Q | W | M | 0.0706 | 1.4317 | 0.0828 | 2664 |
| 2348 | | | | | | | | | | | | | | | | | | |
| Phage- | L | S | N | W | — | V | — | L | — | — | — | E | M | V | 0.0782 | 1.4309 | 0.0796 | 2665 |
| 2349 | | | | | | | | | | | | | | | | | | |
| Phage- | M | D | R | W | — | I | — | E | — | — | — | — | R | H | 0.058 | 1.4307 | 0.0537 | 2666 |
| 2350 | | | | | | | | | | | | | | | | | | |
| Phage- | P | M | K | W | — | I | — | E | — | — | — | I | A | S | 0.0611 | 1.4307 | 0.1022 | 2667 |
| 2351 | | | | | | | | | | | | | | | | | | |
| Phage- | — | D | H | W | — | T | — | A | — | — | — | R | — | — | 0.0571 | 1.4296 | 0.0775 | 2668 |
| 2352 | | | | | | | | | | | | | | | | | | |
| Phage- | S | E | E | W | — | I | — | P | — | — | — | L | — | R | 0.0824 | 1.4288 | 0.0756 | 2669 |
| 2353 | | | | | | | | | | | | | | | | | | |
| Phage- | N | D | H | W | — | I | — | P | — | — | — | — | P | Y | 0.058 | 1.4245 | 0.0707 | 2670 |
| 2354 | | | | | | | | | | | | | | | | | | |
| Phage- | F | E | N | W | — | T | — | S | — | — | — | S | — | — | 0.0597 | 1.4216 | 0.0807 | 2671 |
| 2355 | | | | | | | | | | | | | | | | | | |
| Phage- | V | P | K | W | — | T | — | T | — | — | — | I | H | M | 0.0593 | 1.4208 | 0.0649 | 2672 |
| 2356 | | | | | | | | | | | | | | | | | | |
| Phage- | A | E | R | W | — | I | — | S | — | — | — | E | L | A | 0.0719 | 1.4201 | 0.0756 | 2673 |
| 2357 | | | | | | | | | | | | | | | | | | |
| Phage- | P | V | K | W | — | V | — | P | — | — | — | — | A | T | 0.0718 | 1.4197 | 0.0663 | 2674 |
| 2358 | | | | | | | | | | | | | | | | | | |
| Phage- | A | — | N | W | — | T | — | P | — | — | — | L | — | — | 0.0728 | 1.4192 | 0.0888 | 2675 |
| 2359 | | | | | | | | | | | | | | | | | | |
| Phage- | E | E | K | W | — | R | — | A | — | — | — | E | H | A | 0.0587 | 1.4183 | 0.0625 | 2676 |
| 2360 | | | | | | | | | | | | | | | | | | |
| Phage- | — | T | P | W | — | I | — | P | — | — | — | P | — | P | 0.0592 | 1.4162 | 0.0746 | 2677 |
| 2361 | | | | | | | | | | | | | | | | | | |
| Phage- | E | E | K | M | — | K | — | S | — | — | — | Q | L | — | 0.0938 | 1.4133 | 0.09 | 2678 |
| 2362 | | | | | | | | | | | | | | | | | | |
| Phage- | A | E | R | W | — | I | — | D | — | — | — | N | V | I | 0.0573 | 1.4131 | 0.0567 | 2679 |
| 2363 | | | | | | | | | | | | | | | | | | |
| Phage- | G | — | N | — | — | I | — | P | — | — | — | L | S | S | 0.0549 | 1.4115 | 0.0789 | 2680 |
| 2364 | | | | | | | | | | | | | | | | | | |
| Phage- | A | E | K | — | — | K | — | A | — | — | — | N | N | M | 0.069 | 1.4089 | 0.0752 | 2681 |
| 2365 | | | | | | | | | | | | | | | | | | |
| Phage- | — | N | K | W | — | I | — | E | — | — | — | — | A | M | 0.0612 | 1.4079 | 0.0668 | 2682 |
| 2366 | | | | | | | | | | | | | | | | | | |
| Phage- | Y | E | K | W | — | I | — | A | — | — | — | H | V | A | 0.0601 | 1.4067 | 0.0747 | 2683 |
| 2367 | | | | | | | | | | | | | | | | | | |
| Phage- | D | G | — | W | — | V | — | Q | — | — | — | D | — | Y | 0.0582 | 1.4042 | 0.0722 | 2684 |
| 2368 | | | | | | | | | | | | | | | | | | |
| Phage- | G | L | G | W | — | T | — | T | — | — | — | E | — | — | 0.0535 | 1.402 | 0.0727 | 2685 |
| 2369 | | | | | | | | | | | | | | | | | | |
| Phage- | L | E | K | W | — | I | — | S | — | — | — | S | T | L | 0.0833 | 1.4012 | 0.0829 | 2686 |
| 2370 | | | | | | | | | | | | | | | | | | |
| Phage- | V | D | K | W | — | I | — | S | — | — | — | E | F | Y | 0.0618 | 1.4011 | 0.0767 | 2687 |
| 2371 | | | | | | | | | | | | | | | | | | |
| Phage- | Q | V | Q | W | — | I | — | P | — | — | — | L | E | D | 0.0646 | 1.3989 | 0.0887 | 2688 |
| 2372 | | | | | | | | | | | | | | | | | | |
| Phage- | G | E | K | W | — | I | — | T | — | — | — | D | W | T | 0.0589 | 1.3986 | 0.0544 | 2689 |
| 2373 | | | | | | | | | | | | | | | | | | |
| Phage- | M | G | N | W | — | I | — | E | — | — | — | W | — | T | 0.0665 | 1.3968 | 0.0727 | 2690 |
| 2374 | | | | | | | | | | | | | | | | | | |
| Phage- | D | G | K | W | — | I | — | P | — | — | — | — | M | A | 0.075 | 1.3967 | 0.0859 | 2691 |
| 2375 | | | | | | | | | | | | | | | | | | |
| Phage- | P | D | K | W | — | I | — | Q | — | — | — | D | I | T | 0.0759 | 1.3925 | 0.0617 | 2692 |
| 2376 | | | | | | | | | | | | | | | | | | |
| Phage- | H | D | K | — | — | I | — | L | — | — | — | D | — | V | 0.096 | 1.3915 | 0.0834 | 2693 |
| 2377 | | | | | | | | | | | | | | | | | | |
| Phage- | V | V | K | W | — | I | — | T | — | — | — | Q | T | L | 0.0789 | 1.3914 | 0.1009 | 2694 |
| 2378 | | | | | | | | | | | | | | | | | | |
| Phage- | L | H | K | W | — | T | — | A | — | — | — | E | V | P | 0.0698 | 1.3903 | 0.0904 | 2695 |
| 2379 | | | | | | | | | | | | | | | | | | |
| Phage- | V | Q | K | W | — | I | — | P | — | — | — | Q | I | S | 0.0719 | 1.3902 | 0.0907 | 2696 |
| 2380 | | | | | | | | | | | | | | | | | | |
| Phage- | L | G | I | W | — | I | — | E | — | — | — | V | S | A | 0.0815 | 1.3878 | 0.0793 | 2697 |
| 2381 | | | | | | | | | | | | | | | | | | |
| Phage- | D | F | M | R | — | T | — | E | — | — | — | T | — | — | 0.0649 | 1.3866 | 0.0663 | 2698 |
| 2382 | | | | | | | | | | | | | | | | | | |
| Phage- | F | E | H | W | — | T | — | P | — | — | — | F | S | S | 0.0653 | 1.3836 | 0.0713 | 2699 |
| 2383 | | | | | | | | | | | | | | | | | | |
| Phage- | F | Y | K | W | — | T | — | E | — | — | — | T | V | N | 0.1044 | 1.3833 | 0.1384 | 2700 |
| 2384 | | | | | | | | | | | | | | | | | | |
| Phage- | — | P | K | W | — | T | — | V | — | — | — | — | — | — | 0.057 | 1.3833 | 0.0806 | 2701 |
| 2385 | | | | | | | | | | | | | | | | | | |
| Phage- | V | E | K | W | — | K | — | E | — | — | — | T | S | L | 0.0603 | 1.3832 | 0.0692 | 2702 |
| 2386 | | | | | | | | | | | | | | | | | | |
| Phage- | D | — | K | W | — | I | — | D | — | — | — | Y | — | — | 0.0578 | 1.3816 | 0.0838 | 2703 |
| 2387 | | | | | | | | | | | | | | | | | | |
| Phage- | T | I | K | W | — | T | — | P | — | — | — | I | M | N | 0.0822 | 1.3814 | 0.1027 | 2704 |
| 2388 | | | | | | | | | | | | | | | | | | |
| Phage- | W | V | K | W | — | T | — | P | — | — | — | E | W | D | 0.127 | 1.3813 | 0.1226 | 2705 |
| 2389 | | | | | | | | | | | | | | | | | | |
| Phage- | V | F | H | — | — | M | — | P | — | — | — | S | Q | D | 0.0729 | 1.3801 | 0.0997 | 2706 |
| 2390 | | | | | | | | | | | | | | | | | | |
| Phage- | F | P | K | W | — | T | — | S | — | — | — | D | F | V | 0.0603 | 1.3766 | 0.0984 | 2707 |
| 2391 | | | | | | | | | | | | | | | | | | |
| Phage- | S | P | K | W | — | T | — | S | — | — | — | E | L | Q | 0.0681 | 1.3757 | 0.06 | 2708 |
| 2392 | | | | | | | | | | | | | | | | | | |
| Phage- | L | E | K | W | — | R | — | A | — | — | — | N | — | M | 0.0634 | 1.3754 | 0.0594 | 2709 |
| 2393 | | | | | | | | | | | | | | | | | | |
| Phage- | L | E | R | W | — | I | — | D | — | — | — | E | W | P | 0.054 | 1.3747 | 0.078 | 2710 |
| 2394 | | | | | | | | | | | | | | | | | | |
| Phage- | A | S | M | W | — | I | — | P | — | — | — | — | E | D | 0.0619 | 1.3744 | 0.085 | 2711 |
| 2395 | | | | | | | | | | | | | | | | | | |
| Phage- | E | V | K | W | — | I | — | E | — | — | — | M | V | V | 0.0754 | 1.3737 | 0.0647 | 2712 |
| 2396 | | | | | | | | | | | | | | | | | | |
| Phage- | V | V | K | W | — | T | — | A | — | — | — | E | L | Q | 0.0652 | 1.3735 | 0.0757 | 2713 |
| 2397 | | | | | | | | | | | | | | | | | | |
| Phage- | F | G | I | S | — | T | — | S | — | — | — | E | — | — | 0.069 | 1.3732 | 0.0988 | 2714 |
| 2398 | | | | | | | | | | | | | | | | | | |
| Phage- | W | E | K | W | — | T | — | A | — | — | — | — | V | A | 0.0684 | 1.3716 | 0.0716 | 2715 |
| 2399 | | | | | | | | | | | | | | | | | | |
| Phage- | D | V | R | W | — | T | — | P | — | — | — | V | A | M | 0.0792 | 1.3667 | 0.078 | 2716 |
| 2400 | | | | | | | | | | | | | | | | | | |
| Phage- | M | Q | H | W | — | V | — | P | — | — | — | Q | S | L | 0.0674 | 1.3665 | 0.082 | 2717 |
| 2401 | | | | | | | | | | | | | | | | | | |
| Phage- | V | M | K | W | — | T | — | E | — | — | — | Q | V | — | 0.0558 | 1.366 | 0.0622 | 2718 |
| 2402 | | | | | | | | | | | | | | | | | | |
| Phage- | — | E | K | W | — | I | — | Q | — | — | — | G | N | P | 0.0495 | 1.3612 | 0.0574 | 2719 |
| 2403 | | | | | | | | | | | | | | | | | | |
| Phage- | H | T | T | W | — | T | — | A | — | — | — | Q | — | — | 0.0652 | 1.3601 | 0.0895 | 2720 |
| 2404 | | | | | | | | | | | | | | | | | | |
| Phage- | M | E | K | W | — | I | — | S | — | — | — | V | — | M | 0.0663 | 1.36 | 0.0682 | 2721 |
| 2405 | | | | | | | | | | | | | | | | | | |
| Phage- | P | D | K | W | — | I | — | Q | — | — | — | S | Q | M | 0.0666 | 1.36 | 0.0822 | 2722 |
| 2406 | | | | | | | | | | | | | | | | | | |
| Phage- | T | V | K | W | — | V | — | P | — | — | — | S | V | P | 0.0638 | 1.3595 | 0.0764 | 2723 |
| 2407 | | | | | | | | | | | | | | | | | | |
| Phage- | V | D | H | W | — | T | — | E | — | — | — | V | M | M | 0.0739 | 1.3585 | 0.0963 | 2724 |
| 2408 | | | | | | | | | | | | | | | | | | |
| Phage- | Y | I | K | W | — | T | — | P | — | — | — | E | L | M | 0.0877 | 1.3533 | 0.1496 | 2725 |
| 2409 | | | | | | | | | | | | | | | | | | |
| Phage- | M | H | H | W | — | I | — | A | — | — | — | S | T | E | 0.0683 | 1.3509 | 0.0812 | 2726 |
| 2410 | | | | | | | | | | | | | | | | | | |
| Phage- | Q | — | K | W | — | I | — | Q | — | — | — | E | S | Y | 0.0687 | 1.3489 | 0.0648 | 2727 |
| 2411 | | | | | | | | | | | | | | | | | | |
| Phage- | G | E | K | W | — | I | — | D | — | — | — | — | A | A | 0.063 | 1.3442 | 0.0769 | 2728 |
| 2412 | | | | | | | | | | | | | | | | | | |
| Phage- | L | D | M | R | — | T | — | A | — | — | — | E | — | — | 0.0601 | 1.344 | 0.0768 | 2729 |
| 2413 | | | | | | | | | | | | | | | | | | |
| Phage- | V | E | K | W | — | T | — | P | — | — | — | R | F | D | 0.0656 | 1.3415 | 0.074 | 2730 |
| 2414 | | | | | | | | | | | | | | | | | | |
| Phage- | L | W | K | W | — | T | — | P | — | — | — | E | V | S | 0.0702 | 1.3387 | 0.0864 | 2731 |
| 2415 | | | | | | | | | | | | | | | | | | |
| Phage- | S | E | R | W | — | I | — | Q | — | — | — | T | A | P | 0.0609 | 1.3385 | 0.0763 | 2732 |
| 2416 | | | | | | | | | | | | | | | | | | |
| Phage- | F | D | P | W | — | I | — | L | — | — | — | E | — | M | 0.0556 | 1.3376 | 0.0667 | 2733 |
| 2417 | | | | | | | | | | | | | | | | | | |
| Phage- | F | S | K | — | — | I | — | A | — | — | — | S | E | E | 0.0936 | 1.3369 | 0.0956 | 2734 |
| 2418 | | | | | | | | | | | | | | | | | | |
| Phage- | V | — | K | W | — | I | — | S | — | — | — | E | T | V | 0.077 | 1.3341 | 0.0735 | 2735 |
| 2419 | | | | | | | | | | | | | | | | | | |
| Phage- | E | I | K | W | — | I | — | S | — | — | — | N | T | L | 0.0672 | 1.3338 | 0.0772 | 2736 |
| 2420 | | | | | | | | | | | | | | | | | | |
| Phage- | E | F | K | W | — | I | — | K | — | — | — | M | V | D | 0.0909 | 1.3329 | 0.0948 | 2737 |
| 2421 | | | | | | | | | | | | | | | | | | |
| Phage- | W | T | N | T | — | I | — | M | — | — | — | E | — | — | 0.0746 | 1.3326 | 0.0808 | 2738 |
| 2422 | | | | | | | | | | | | | | | | | | |
| Phage- | L | T | N | W | — | T | — | Q | — | — | — | L | — | — | 0.0601 | 1.3318 | 0.0731 | 2739 |
| 2423 | | | | | | | | | | | | | | | | | | |
| Phage- | L | V | N | W | — | T | — | P | — | — | — | W | H | D | 0.0724 | 1.3314 | 0.0827 | 2740 |
| 2424 | | | | | | | | | | | | | | | | | | |
| Phage- | D | E | N | W | — | K | — | L | — | — | — | E | — | — | 0.0812 | 1.3306 | 0.0991 | 2741 |
| 2425 | | | | | | | | | | | | | | | | | | |
| Phage- | L | W | M | S | — | I | — | P | — | — | — | — | N | V | 0.1172 | 1.3305 | 0.1368 | 2742 |
| 2426 | | | | | | | | | | | | | | | | | | |
| Phage- | E | Y | K | W | — | V | — | I | — | — | — | — | Y | D | 0.0684 | 1.33 | 0.0826 | 2743 |
| 2427 | | | | | | | | | | | | | | | | | | |
| Phage- | A | D | R | — | — | I | — | P | — | — | — | E | M | K | 0.0626 | 1.3277 | 0.0685 | 2744 |
| 2428 | | | | | | | | | | | | | | | | | | |
| Phage- | G | E | K | W | — | I | — | E | — | — | — | V | T | T | 0.0558 | 1.3211 | 0.0637 | 2745 |
| 2429 | | | | | | | | | | | | | | | | | | |
| Phage- | E | I | K | W | — | T | — | P | — | — | — | T | I | G | 0.0727 | 1.3207 | 0.1022 | 2746 |
| 2430 | | | | | | | | | | | | | | | | | | |
| Phage- | V | L | K | W | — | T | — | P | — | — | — | W | D | I | 0.0945 | 1.32 | 0.1231 | 2747 |
| 2431 | | | | | | | | | | | | | | | | | | |
| Phage- | E | V | K | W | — | I | — | — | — | — | — | — | A | S | 0.0603 | 1.3191 | 0.077 | 2748 |
| 2432 | | | | | | | | | | | | | | | | | | |
| Phage- | G | D | N | W | — | K | — | A | — | — | — | R | D | M | 0.0603 | 1.3163 | 0.0728 | 2749 |
| 2433 | | | | | | | | | | | | | | | | | | |
| Phage- | E | E | R | W | — | I | — | E | — | — | — | S | R | I | 0.0524 | 1.3158 | 0.0557 | 2750 |
| 2434 | | | | | | | | | | | | | | | | | | |
| Phage- | G | I | N | W | — | T | — | S | — | — | — | T | — | — | 0.0593 | 1.3137 | 0.0711 | 2751 |
| 2435 | | | | | | | | | | | | | | | | | | |
| Phage- | M | P | K | W | — | T | — | S | — | — | — | S | P | S | 0.0579 | 1.3125 | 0.0576 | 2752 |
| 2436 | | | | | | | | | | | | | | | | | | |
| Phage- | E | M | N | W | — | I | — | A | — | — | — | E | — | R | 0.0572 | 1.3116 | 0.0633 | 2753 |
| 2437 | | | | | | | | | | | | | | | | | | |
| Phage- | E | — | K | W | — | I | — | — | — | — | — | E | M | A | 0.0605 | 1.3115 | 0.0622 | 2754 |
| 2438 | | | | | | | | | | | | | | | | | | |
| Phage- | M | P | K | W | — | T | — | S | — | — | — | — | V | Q | 0.0573 | 1.3096 | 0.0845 | 2755 |
| 2439 | | | | | | | | | | | | | | | | | | |
| Phage- | P | — | K | W | — | V | — | S | — | — | — | S | E | I | 0.0561 | 1.3088 | 0.0676 | 2756 |
| 2440 | | | | | | | | | | | | | | | | | | |
| Phage- | V | V | H | W | — | T | — | A | — | — | — | Y | D | M | 0.0554 | 1.3043 | 0.0623 | 2757 |
| 2441 | | | | | | | | | | | | | | | | | | |
| Phage- | L | H | K | — | — | T | — | T | — | — | — | E | — | — | 0.0638 | 1.2988 | 0.0712 | 2758 |
| 2442 | | | | | | | | | | | | | | | | | | |
| Phage- | G | — | I | W | — | I | — | Q | — | — | — | — | W | N | 0.1201 | 1.2966 | 0.1323 | 2759 |
| 2443 | | | | | | | | | | | | | | | | | | |
| Phage- | E | V | K | W | — | I | — | A | — | — | — | V | E | D | 0.0701 | 1.296 | 0.0892 | 2760 |
| 2444 | | | | | | | | | | | | | | | | | | |
| Phage- | L | E | R | W | — | I | — | S | — | — | — | T | E | D | 0.063 | 1.2955 | 0.0674 | 2761 |
| 2445 | | | | | | | | | | | | | | | | | | |
| Phage- | Y | E | K | W | — | T | — | T | — | — | — | S | W | L | 0.1368 | 1.2922 | 0.2176 | 2762 |
| 2446 | | | | | | | | | | | | | | | | | | |
| Phage- | — | E | K | W | — | T | — | P | — | — | — | L | D | H | 0.0621 | 1.291 | 0.0663 | 2763 |
| 2447 | | | | | | | | | | | | | | | | | | |
| Phage- | E | S | K | W | — | I | — | E | — | — | — | E | Q | R | 0.0579 | 1.2884 | 0.0688 | 2764 |
| 2448 | | | | | | | | | | | | | | | | | | |
| Phage- | E | E | P | W | — | I | — | S | — | — | — | — | L | T | 0.0547 | 1.2879 | 0.0689 | 2765 |
| 2449 | | | | | | | | | | | | | | | | | | |
| Phage- | V | V | K | W | — | T | — | P | — | — | — | S | V | W | 0.0858 | 1.2876 | 0.1038 | 2766 |
| 2450 | | | | | | | | | | | | | | | | | | |
| Phage- | W | D | H | — | — | K | — | R | — | — | — | D | — | I | 0.0757 | 1.2809 | 0.0744 | 2767 |
| 2451 | | | | | | | | | | | | | | | | | | |
| Phage- | S | V | K | M | — | I | — | E | — | — | — | E | N | I | 0.0603 | 1.2755 | 0.0646 | 2768 |
| 2452 | | | | | | | | | | | | | | | | | | |
| Phage- | V | E | T | W | — | I | — | H | — | — | — | — | — | — | 0.0632 | 1.2727 | 0.0685 | 2769 |
| 2453 | | | | | | | | | | | | | | | | | | |
| Phage- | P | T | K | — | — | — | — | P | — | — | — | Y | P | S | 0.0585 | 1.2708 | 0.0754 | 2770 |
| 2454 | | | | | | | | | | | | | | | | | | |
| Phage- | S | V | H | E | — | R | — | L | — | — | — | Q | — | — | 0.0636 | 1.27 | 0.073 | 2771 |
| 2455 | | | | | | | | | | | | | | | | | | |
| Phage- | G | E | K | W | — | I | — | S | — | — | — | V | A | V | 0.0549 | 1.2673 | 0.0549 | 2772 |
| 2456 | | | | | | | | | | | | | | | | | | |
| Phage- | V | E | I | — | — | K | — | A | — | — | — | R | L | D | 0.0827 | 1.2673 | 0.062 | 2773 |
| 2457 | | | | | | | | | | | | | | | | | | |
| Phage- | D | T | N | W | — | R | — | A | — | — | — | — | F | A | 0.0541 | 1.267 | 0.0649 | 2774 |
| 2458 | | | | | | | | | | | | | | | | | | |
| Phage- | T | Q | M | W | — | I | — | F | — | — | — | E | V | Q | 0.0758 | 1.2665 | 0.0885 | 2775 |
| 2459 | | | | | | | | | | | | | | | | | | |
| Phage- | E | M | K | W | — | T | — | P | — | — | — | T | E | L | 0.0566 | 1.258 | 0.0656 | 2776 |
| 2460 | | | | | | | | | | | | | | | | | | |
| Phage- | S | V | K | — | — | I | — | D | — | — | — | V | S | E | 0.147 | 1.2563 | 0.2033 | 2777 |
| 2461 | | | | | | | | | | | | | | | | | | |
| Phage- | D | E | I | W | — | I | — | R | — | — | — | D | — | I | 0.0587 | 1.2548 | 0.0834 | 2778 |
| 2462 | | | | | | | | | | | | | | | | | | |
| Phage- | D | G | K | W | — | I | — | E | — | — | — | — | Q | Q | 0.0593 | 1.2484 | 0.0837 | 2779 |
| 2463 | | | | | | | | | | | | | | | | | | |
| Phage- | P | V | K | — | — | K | — | D | — | — | — | W | V | S | 0.0763 | 1.2481 | 0.1055 | 2780 |
| 2464 | | | | | | | | | | | | | | | | | | |
| Phage- | V | M | K | W | — | T | — | Q | — | — | — | E | H | L | 0.0577 | 1.2458 | 0.068 | 2781 |
| 2465 | | | | | | | | | | | | | | | | | | |
| Phage- | V | V | K | W | — | T | — | S | — | — | — | T | — | — | 0.0568 | 1.243 | 0.0837 | 2782 |
| 2466 | | | | | | | | | | | | | | | | | | |
| Phage- | G | D | K | W | — | I | — | D | — | — | — | V | Q | S | 0.0619 | 1.2414 | 0.0692 | 2783 |
| 2467 | | | | | | | | | | | | | | | | | | |
| Phage- | F | E | K | W | — | I | — | A | — | — | — | E | Q | V | 0.06 | 1.2413 | 0.0596 | 2784 |
| 2468 | | | | | | | | | | | | | | | | | | |
| Phage- | V | S | K | W | — | T | — | A | — | — | — | S | A | M | 0.0787 | 1.2403 | 0.099 | 2785 |
| 2469 | | | | | | | | | | | | | | | | | | |
| Phage- | V | D | K | — | — | R | — | A | — | — | — | V | A | S | 0.0637 | 1.2399 | 0.0775 | 2786 |
| 2470 | | | | | | | | | | | | | | | | | | |
| Phage- | M | E | K | W | — | I | — | D | — | — | — | L | — | N | 0.1357 | 1.2397 | 0.0809 | 2787 |
| 2471 | | | | | | | | | | | | | | | | | | |
| Phage- | F | D | K | W | — | I | — | P | — | — | — | V | — | P | 0.0559 | 1.2396 | 0.0689 | 2788 |
| 2472 | | | | | | | | | | | | | | | | | | |
| Phage- | P | E | H | W | — | I | — | P | — | — | — | V | V | H | 0.0917 | 1.2389 | 0.513 | 2789 |
| 2473 | | | | | | | | | | | | | | | | | | |
| Phage- | D | F | H | M | — | T | — | R | — | — | — | E | — | M | 0.0518 | 1.2382 | 0.0622 | 2790 |
| 2474 | | | | | | | | | | | | | | | | | | |
| Phage- | E | H | K | W | — | T | — | L | — | — | — | E | — | L | 0.0706 | 1.2348 | 0.09 | 2791 |
| 2475 | | | | | | | | | | | | | | | | | | |
| Phage- | Y | H | N | W | — | V | — | Q | — | — | — | V | — | L | 0.0788 | 1.2342 | 0.1268 | 2792 |
| 2476 | | | | | | | | | | | | | | | | | | |
| Phage- | V | E | K | W | — | I | — | Q | — | — | — | S | M | D | 0.0613 | 1.2342 | 0.0715 | 2793 |
| 2477 | | | | | | | | | | | | | | | | | | |
| Phage- | E | M | I | R | — | I | — | D | — | — | — | M | S | Y | 0.0593 | 1.2341 | 0.0624 | 2794 |
| 2478 | | | | | | | | | | | | | | | | | | |
| Phage- | G | F | P | M | — | K | — | L | — | — | — | E | N | L | 0.0602 | 1.2317 | 0.0667 | 2795 |
| 2479 | | | | | | | | | | | | | | | | | | |
| Phage- | P | F | K | W | — | T | — | A | — | — | — | E | V | H | 0.0557 | 1.2317 | 0.0594 | 2796 |
| 2480 | | | | | | | | | | | | | | | | | | |
| Phage- | W | S | N | W | — | I | — | P | — | — | — | — | M | N | 0.0583 | 1.2285 | 0.0771 | 2797 |
| 2481 | | | | | | | | | | | | | | | | | | |
| Phage- | V | V | N | W | — | T | — | A | — | — | — | S | — | — | 0.0898 | 1.2277 | 0.0652 | 2798 |
| 2482 | | | | | | | | | | | | | | | | | | |
| Phage- | D | S | R | W | — | I | — | D | — | — | — | E | L | E | 0.0545 | 1.2259 | 0.0935 | 2799 |
| 2483 | | | | | | | | | | | | | | | | | | |
| Phage- | L | V | R | W | — | V | — | E | — | — | — | — | — | M | 0.0663 | 1.2246 | 0.0945 | 2800 |
| 2484 | | | | | | | | | | | | | | | | | | |
| Phage- | V | S | P | — | — | K | — | D | — | — | — | N | — | — | 0.0545 | 1.2241 | 0.0787 | 2801 |
| 2485 | | | | | | | | | | | | | | | | | | |
| Phage- | F | P | H | — | — | I | — | Y | — | — | — | V | R | T | 0.1477 | 1.2235 | 0.1981 | 2802 |
| 2486 | | | | | | | | | | | | | | | | | | |
| Phage- | D | Y | K | W | — | T | — | I | — | — | — | — | — | D | 0.0586 | 1.2227 | 0.0707 | 2803 |
| 2487 | | | | | | | | | | | | | | | | | | |
| Phage- | V | S | I | W | — | I | — | P | — | — | — | V | E | K | 0.0729 | 1.221 | 0.101 | 2804 |
| 2488 | | | | | | | | | | | | | | | | | | |
| Phage- | W | — | K | W | — | T | — | E | — | — | — | V | V | H | 0.1291 | 1.2209 | 0.1499 | 2805 |
| 2489 | | | | | | | | | | | | | | | | | | |
| Phage- | D | F | K | W | — | T | — | P | — | — | — | D | V | — | 0.068 | 1.2185 | 0.0763 | 2806 |
| 2490 | | | | | | | | | | | | | | | | | | |
| Phage- | S | V | K | W | — | T | — | S | — | — | — | E | H | M | 0.0674 | 1.2183 | 0.0739 | 2807 |
| 2491 | | | | | | | | | | | | | | | | | | |
| Phage- | — | E | K | M | — | I | — | A | — | — | — | T | D | K | 0.0532 | 1.2179 | 0.0698 | 2808 |
| 2492 | | | | | | | | | | | | | | | | | | |
| Phage- | M | G | N | W | — | T | — | P | — | — | — | T | — | — | 0.0714 | 1.2178 | 0.0704 | 2809 |
| 2493 | | | | | | | | | | | | | | | | | | |
| Phage- | H | F | K | M | — | V | — | P | — | — | — | L | F | D | 0.0719 | 1.2176 | 0.1014 | 2810 |
| 2494 | | | | | | | | | | | | | | | | | | |
| Phage- | L | E | K | W | — | I | — | L | — | — | — | Q | T | A | 0.0551 | 1.2175 | 0.0777 | 2811 |
| 2495 | | | | | | | | | | | | | | | | | | |
| Phage- | E | S | R | — | — | I | — | A | — | — | — | E | S | Q | 0.0673 | 1.2171 | 0.0772 | 2812 |
| 2496 | | | | | | | | | | | | | | | | | | |
| Phage- | W | P | K | — | — | T | — | P | — | — | — | T | V | D | 0.0893 | 1.2126 | 0.0722 | 2813 |
| 2497 | | | | | | | | | | | | | | | | | | |
| Phage- | G | E | H | W | — | I | — | A | — | — | — | E | K | M | 0.0637 | 1.2083 | 0.0615 | 2814 |
| 2498 | | | | | | | | | | | | | | | | | | |
| Phage- | G | E | K | W | — | I | — | E | — | — | — | Q | D | V | 0.0591 | 1.2077 | 0.0722 | 2815 |
| 2499 | | | | | | | | | | | | | | | | | | |
| Phage- | G | T | N | W | — | K | — | D | — | — | — | V | — | L | 0.0647 | 1.2068 | 0.0855 | 2816 |
| 2500 | | | | | | | | | | | | | | | | | | |
| Phage- | S | D | K | W | — | I | — | V | — | — | — | E | E | S | 0.0631 | 1.2061 | 0.0874 | 2817 |
| 2501 | | | | | | | | | | | | | | | | | | |
| Phage- | E | P | K | M | — | T | — | S | — | — | — | R | — | — | 0.0811 | 1.2034 | 0.0691 | 2818 |
| 2502 | | | | | | | | | | | | | | | | | | |
| Phage- | S | E | T | W | — | I | — | A | — | — | — | L | D | G | 0.0646 | 1.2019 | 0.0745 | 2819 |
| 2503 | | | | | | | | | | | | | | | | | | |
| Phage- | — | P | K | W | — | T | — | S | — | — | — | E | W | V | 0.0655 | 1.2009 | 0.0916 | 2820 |
| 2504 | | | | | | | | | | | | | | | | | | |
| Phage- | L | G | K | W | — | I | — | D | — | — | — | S | — | L | 0.0924 | 1.2007 | 0.1442 | 2821 |
| 2505 | | | | | | | | | | | | | | | | | | |
| Phage- | V | Q | M | — | — | I | — | A | — | — | — | G | M | E | 0.0729 | 1.1965 | 0.1071 | 2822 |
| 2506 | | | | | | | | | | | | | | | | | | |
| Phage- | V | E | R | — | — | K | — | L | — | — | — | Q | E | R | 0.065 | 1.1961 | 0.0748 | 2823 |
| 2507 | | | | | | | | | | | | | | | | | | |
| Phage- | H | P | S | W | — | T | — | A | — | — | — | Q | — | — | 0.0621 | 1.1956 | 0.0911 | 2824 |
| 2508 | | | | | | | | | | | | | | | | | | |
| Phage- | T | Y | K | M | — | I | — | A | — | K | — | — | V | S | 0.0608 | 1.1936 | 0.0792 | 2825 |
| 2509 | | | | | | | | | | | | | | | | | | |
| Phage- | E | V | K | W | — | I | — | D | — | — | — | S | V | I | 0.0683 | 1.1904 | 0.0782 | 2826 |
| 2510 | | | | | | | | | | | | | | | | | | |
| Phage- | L | V | K | — | — | S | — | E | — | — | — | V | — | M | 0.077 | 1.1893 | 0.0667 | 2827 |
| 2511 | | | | | | | | | | | | | | | | | | |
| Phage- | L | G | K | M | — | I | — | L | — | — | — | D | — | L | 0.0751 | 1.1869 | 0.0573 | 2828 |
| 2512 | | | | | | | | | | | | | | | | | | |
| Phage- | S | L | E | — | — | R | — | E | — | — | — | V | — | — | 0.0599 | 1.1868 | 0.0955 | 2829 |
| 2513 | | | | | | | | | | | | | | | | | | |
| Phage- | E | I | K | W | — | T | — | A | — | — | — | — | I | N | 0.0732 | 1.1841 | 0.0972 | 2830 |
| 2514 | | | | | | | | | | | | | | | | | | |
| Phage- | L | T | Q | W | — | I | — | Q | — | — | — | E | — | S | 0.0574 | 1.1831 | 0.0537 | 2831 |
| 2515 | | | | | | | | | | | | | | | | | | |
| Phage- | V | P | K | W | — | T | — | A | — | — | — | T | — | L | 0.0638 | 1.1826 | 0.0721 | 2832 |
| 2516 | | | | | | | | | | | | | | | | | | |
| Phage- | V | D | N | W | — | T | — | M | — | — | — | — | W | I | 0.0855 | 1.1822 | 0.1094 | 2833 |
| 2517 | | | | | | | | | | | | | | | | | | |
| Phage- | — | D | P | R | — | I | — | E | — | — | — | I | — | Y | 0.0538 | 1.1819 | 0.0633 | 2834 |
| 2518 | | | | | | | | | | | | | | | | | | |
| Phage- | V | P | I | W | — | T | — | E | — | — | — | S | — | — | 0.0637 | 1.1799 | 0.0879 | 2835 |
| 2519 | | | | | | | | | | | | | | | | | | |
| Phage- | P | I | P | — | — | I | — | L | — | — | — | E | E | R | 0.0585 | 1.179 | 0.0838 | 2836 |
| 2520 | | | | | | | | | | | | | | | | | | |
| Phage- | D | N | K | W | — | I | — | E | — | — | — | F | A | S | 0.0563 | 1.1784 | 0.0796 | 2837 |
| 2521 | | | | | | | | | | | | | | | | | | |
| Phage- | H | E | R | S | — | I | — | A | — | — | — | W | R | Q | 0.0714 | 1.1779 | 0.0706 | 2838 |
| 2522 | | | | | | | | | | | | | | | | | | |
| Phage- | — | V | R | W | — | T | — | E | — | — | — | V | — | — | 0.0622 | 1.1766 | 0.0674 | 2839 |
| 2523 | | | | | | | | | | | | | | | | | | |
| Phage- | A | V | K | W | — | T | — | A | — | — | — | S | — | — | 0.0524 | 1.1757 | 0.0682 | 2840 |
| 2524 | | | | | | | | | | | | | | | | | | |
| Phage- | V | I | T | W | — | T | — | P | — | — | — | S | — | W | 0.0855 | 1.1727 | 0.0911 | 2841 |
| 2525 | | | | | | | | | | | | | | | | | | |
| Phage- | W | E | K | W | — | T | — | S | — | — | — | S | A | G | 0.0713 | 1.1697 | 0.0743 | 2842 |
| 2526 | | | | | | | | | | | | | | | | | | |
| Phage- | L | G | H | W | — | T | — | P | — | — | — | T | E | S | 0.0613 | 1.1696 | 0.0769 | 2843 |
| 2527 | | | | | | | | | | | | | | | | | | |
| Phage- | Y | E | K | W | — | T | — | A | — | — | — | D | F | V | 0.0604 | 1.1683 | 0.07 | 2844 |
| 2528 | | | | | | | | | | | | | | | | | | |
| Phage- | — | V | K | W | — | T | — | H | — | — | — | S | — | W | 0.1483 | 1.1661 | 0.1368 | 2845 |
| 2529 | | | | | | | | | | | | | | | | | | |
| Phage- | V | T | H | W | — | I | — | E | — | — | — | V | V | E | 0.0602 | 1.1661 | 0.065 | 2846 |
| 2530 | | | | | | | | | | | | | | | | | | |
| Phage- | Y | E | K | W | — | I | — | R | — | — | — | E | A | N | 0.069 | 1.1612 | 0.0742 | 2847 |
| 2531 | | | | | | | | | | | | | | | | | | |
| Phage- | E | H | R | W | — | I | — | V | — | — | — | E | K | S | 0.066 | 1.161 | 0.0675 | 2848 |
| 2532 | | | | | | | | | | | | | | | | | | |
| Phage- | P | V | P | — | — | R | — | P | — | — | — | Q | S | V | 0.0576 | 1.1602 | 0.0797 | 2849 |
| 2533 | | | | | | | | | | | | | | | | | | |
| Phage- | V | P | K | W | — | I | — | V | — | — | — | E | V | M | 0.0848 | 1.1598 | 0.1111 | 2850 |
| 2534 | | | | | | | | | | | | | | | | | | |
| Phage- | D | W | K | W | — | T | — | A | — | — | — | S | T | E | 0.0689 | 1.1585 | 0.1168 | 2851 |
| 2535 | | | | | | | | | | | | | | | | | | |
| Phage- | V | S | P | K | — | I | — | P | — | — | — | S | Y | V | 0.0564 | 1.1574 | 0.0685 | 2852 |
| 2536 | | | | | | | | | | | | | | | | | | |
| Phage- | E | M | K | W | — | I | — | Q | — | — | — | — | D | S | 0.0576 | 1.1571 | 0.0656 | 2853 |
| 2537 | | | | | | | | | | | | | | | | | | |
| Phage- | E | P | K | W | — | V | — | P | — | — | — | — | Y | D | 0.0621 | 1.1567 | 0.0994 | 2854 |
| 2538 | | | | | | | | | | | | | | | | | | |
| Phage- | L | M | N | W | — | I | — | S | — | — | — | D | V | E | 0.0729 | 1.1545 | 0.0733 | 2855 |
| 2539 | | | | | | | | | | | | | | | | | | |
| Phage- | E | M | K | W | — | T | — | E | — | — | — | I | — | L | 0.1184 | 1.1525 | 0.3126 | 2856 |
| 2540 | | | | | | | | | | | | | | | | | | |
| Phage- | P | T | K | W | — | I | — | E | — | — | — | V | E | S | 0.0529 | 1.1504 | 0.0567 | 2857 |
| 2541 | | | | | | | | | | | | | | | | | | |
| Phage- | D | L | K | W | — | F | — | D | — | — | — | Q | V | G | 0.0647 | 1.1504 | 0.0841 | 2858 |
| 2542 | | | | | | | | | | | | | | | | | | |
| Phage- | S | E | H | W | — | I | — | Q | — | — | — | Q | S | V | 0.0802 | 1.1465 | 0.1138 | 2859 |
| 2543 | | | | | | | | | | | | | | | | | | |
| Phage- | P | L | K | W | — | I | — | Q | — | — | — | T | E | A | 0.0531 | 1.1462 | 0.0825 | 2860 |
| 2544 | | | | | | | | | | | | | | | | | | |
| Phage- | L | Y | N | W | — | T | — | A | — | — | — | Y | — | Y | 0.1958 | 1.1453 | 0.2841 | 2861 |
| 2545 | | | | | | | | | | | | | | | | | | |
| Phage- | E | S | H | W | — | I | — | A | — | — | — | E | Q | — | 0.0574 | 1.1444 | 0.0589 | 2862 |
| 2546 | | | | | | | | | | | | | | | | | | |
| Phage- | E | G | K | W | — | I | — | D | — | — | — | — | A | H | 0.0544 | 1.1443 | 0.0598 | 2863 |
| 2547 | | | | | | | | | | | | | | | | | | |
| Phage- | F | E | N | W | — | T | — | K | — | — | — | I | — | V | 0.072 | 1.1432 | 0.1017 | 2864 |
| 2548 | | | | | | | | | | | | | | | | | | |
| Phage- | V | Q | K | W | — | I | — | P | — | — | — | T | H | I | 0.0713 | 1.1368 | 0.0958 | 2865 |
| 2549 | | | | | | | | | | | | | | | | | | |
| Phage- | F | H | E | W | — | I | — | S | — | — | — | F | K | E | 0.0803 | 1.1355 | 0.0984 | 2866 |
| 2550 | | | | | | | | | | | | | | | | | | |
| Phage- | E | P | K | W | — | T | — | P | — | — | — | Q | S | V | 0.101 | 1.1355 | 0.0734 | 2867 |
| 2551 | | | | | | | | | | | | | | | | | | |
| Phage- | A | E | Y | W | — | I | — | R | — | — | — | S | Q | H | 0.0657 | 1.135 | 0.0882 | 2868 |
| 2552 | | | | | | | | | | | | | | | | | | |
| Phage- | G | E | K | A | — | I | — | A | — | — | — | S | E | N | 0.0556 | 1.1278 | 0.0736 | 2869 |
| 2553 | | | | | | | | | | | | | | | | | | |
| Phage- | M | F | K | W | — | T | — | Q | — | — | — | E | T | M | 0.0969 | 1.1253 | 0.1243 | 2870 |
| 2554 | | | | | | | | | | | | | | | | | | |
| Phage- | T | Q | K | — | — | I | — | E | — | — | — | E | A | M | 0.0653 | 1.1244 | 0.0698 | 2871 |
| 2555 | | | | | | | | | | | | | | | | | | |
| Phage- | L | Q | T | W | — | I | — | P | — | — | — | E | — | G | 0.0633 | 1.1243 | 0.0675 | 2872 |
| 2556 | | | | | | | | | | | | | | | | | | |
| Phage- | V | L | K | W | — | T | — | A | — | — | — | T | — | — | 0.0747 | 1.1242 | 0.0805 | 2873 |
| 2557 | | | | | | | | | | | | | | | | | | |
| Phage- | W | E | N | W | — | T | — | T | — | — | — | W | P | N | 0.0648 | 1.1231 | 0.0834 | 2874 |
| 2558 | | | | | | | | | | | | | | | | | | |
| Phage- | W | D | K | W | — | T | — | P | — | — | — | Q | V | H | 0.0599 | 1.1211 | 0.0758 | 2875 |
| 2559 | | | | | | | | | | | | | | | | | | |
| Phage- | G | V | M | W | — | I | — | Q | — | — | — | V | T | A | 0.0765 | 1.1183 | 0.1039 | 2876 |
| 2560 | | | | | | | | | | | | | | | | | | |
| Phage- | Y | — | P | W | — | I | — | L | — | — | — | L | D | R | 0.0939 | 1.114 | 0.1606 | 2877 |
| 2561 | | | | | | | | | | | | | | | | | | |
| Phage- | — | — | K | — | — | T | — | A | — | — | — | E | R | H | 0.0971 | 1.114 | 0.0803 | 2878 |
| 2562 | | | | | | | | | | | | | | | | | | |
| Phage- | N | W | K | W | — | I | — | E | — | — | — | — | A | E | 0.0551 | 1.1129 | 0.0635 | 2879 |
| 2563 | | | | | | | | | | | | | | | | | | |
| Phage- | S | E | I | W | — | I | — | A | — | — | — | Q | S | T | 0.0622 | 1.1121 | 0.0851 | 2880 |
| 2564 | | | | | | | | | | | | | | | | | | |
| Phage- | V | E | K | W | — | I | — | E | — | — | — | W | T | V | 0.061 | 1.1103 | 0.0709 | 2881 |
| 2565 | | | | | | | | | | | | | | | | | | |
| Phage- | V | D | P | W | — | K | — | A | — | — | — | L | — | M | 0.0593 | 1.108 | 0.0597 | 2882 |
| 2566 | | | | | | | | | | | | | | | | | | |
| Phage- | E | P | K | W | — | T | — | P | — | — | — | E | N | — | 0.0605 | 1.1062 | 0.0576 | 2883 |
| 2567 | | | | | | | | | | | | | | | | | | |
| Phage- | Y | D | — | — | — | K | — | D | — | — | — | N | W | N | 0.0698 | 1.1058 | 0.0861 | 2884 |
| 2568 | | | | | | | | | | | | | | | | | | |
| Phage- | A | I | H | W | — | T | — | P | — | — | — | E | P | Q | 0.0685 | 1.1018 | 0.0777 | 2885 |
| 2569 | | | | | | | | | | | | | | | | | | |
| Phage- | E | G | P | W | — | I | — | E | — | — | — | Q | N | S | 0.0695 | 1.0986 | 0.0766 | 2886 |
| 2570 | | | | | | | | | | | | | | | | | | |
| Phage- | — | — | K | W | — | I | — | S | — | — | — | — | — | I | 0.1156 | 1.0982 | 0.139 | 2887 |
| 2571 | | | | | | | | | | | | | | | | | | |
| Phage- | Q | Y | N | W | — | V | — | A | — | — | — | E | I | S | 0.0574 | 1.0958 | 0.0848 | 2888 |
| 2572 | | | | | | | | | | | | | | | | | | |
| Phage- | V | D | T | W | — | T | — | P | — | — | — | W | — | L | 0.0631 | 1.0923 | 0.0695 | 2889 |
| 2573 | | | | | | | | | | | | | | | | | | |
| Phage- | T | F | K | W | — | T | — | A | — | — | — | — | — | — | 0.0631 | 1.0917 | 0.0801 | 2890 |
| 2574 | | | | | | | | | | | | | | | | | | |
| Phage- | E | F | K | W | — | T | — | E | — | — | — | — | M | A | 0.0599 | 1.091 | 0.0845 | 2891 |
| 2575 | | | | | | | | | | | | | | | | | | |
| Phage- | Q | E | K | M | — | I | — | A | — | — | — | S | E | Y | 0.0583 | 1.0863 | 0.0615 | 2892 |
| 2576 | | | | | | | | | | | | | | | | | | |
| Phage- | L | T | N | W | — | T | — | S | — | — | — | Y | — | L | 0.0634 | 1.0853 | 0.0817 | 2893 |
| 2577 | | | | | | | | | | | | | | | | | | |
| Phage- | L | Q | E | W | — | I | — | P | — | — | — | I | K | S | 0.0612 | 1.0853 | 0.0797 | 2894 |
| 2578 | | | | | | | | | | | | | | | | | | |
| Phage- | G | P | K | W | — | T | — | P | — | — | — | W | E | — | 0.0592 | 1.0819 | 0.3313 | 2895 |
| 2579 | | | | | | | | | | | | | | | | | | |
| Phage- | G | V | K | W | — | T | — | A | — | — | — | E | W | T | 0.0602 | 1.0807 | 0.0707 | 2896 |
| 2580 | | | | | | | | | | | | | | | | | | |
| Phage- | S | E | K | W | — | I | — | E | — | — | — | M | Y | A | 0.0491 | 1.076 | 0.0532 | 2897 |
| 2581 | | | | | | | | | | | | | | | | | | |
| Phage- | E | G | K | W | — | I | — | E | — | — | — | I | S | V | 0.068 | 1.0759 | 0.0637 | 2898 |
| 2582 | | | | | | | | | | | | | | | | | | |
| Phage- | E | V | M | W | — | R | — | A | — | — | — | D | — | I | 0.0555 | 1.0748 | 0.0623 | 2899 |
| 2583 | | | | | | | | | | | | | | | | | | |
| Phage- | V | V | K | W | — | K | — | A | — | — | — | E | Q | A | 0.0628 | 1.0709 | 0.0788 | 2900 |
| 2584 | | | | | | | | | | | | | | | | | | |
| Phage- | D | L | K | R | — | I | — | D | — | — | — | — | V | A | 0.06 | 1.0661 | 0.0617 | 2901 |
| 2585 | | | | | | | | | | | | | | | | | | |
| Phage- | E | T | Q | M | — | V | — | Q | — | — | — | W | — | I | 0.0519 | 1.0654 | 0.0571 | 2902 |
| 2586 | | | | | | | | | | | | | | | | | | |
| Phage- | V | G | K | W | — | V | — | M | — | — | — | — | T | D | 0.0731 | 1.0625 | 0.0735 | 2903 |
| 2587 | | | | | | | | | | | | | | | | | | |
| Phage- | M | I | G | W | — | T | — | S | — | — | — | S | — | — | 0.0647 | 1.0594 | 0.0716 | 2904 |
| 2588 | | | | | | | | | | | | | | | | | | |
| Phage- | V | L | N | W | — | T | — | P | — | — | — | L | — | I | 0.0805 | 1.0568 | 0.107 | 2905 |
| 2589 | | | | | | | | | | | | | | | | | | |
| Phage- | M | P | R | W | — | T | — | A | — | — | — | E | A | I | 0.0594 | 1.0541 | 0.063 | 2906 |
| 2590 | | | | | | | | | | | | | | | | | | |
| Phage- | T | D | K | W | — | V | — | E | — | — | — | M | E | — | 0.0535 | 1.0539 | 0.0773 | 2907 |
| 2591 | | | | | | | | | | | | | | | | | | |
| Phage- | H | F | N | W | — | T | — | S | — | — | — | D | V | L | 0.0858 | 1.0534 | 0.1181 | 2908 |
| 2592 | | | | | | | | | | | | | | | | | | |
| Phage- | D | G | I | W | — | T | — | R | — | — | — | E | — | M | 0.0668 | 1.0505 | 0.0865 | 2909 |
| 2593 | | | | | | | | | | | | | | | | | | |
| Phage- | H | P | Q | — | — | I | — | S | — | — | — | N | V | W | 0.0587 | 1.0492 | 0.1048 | 2910 |
| 2594 | | | | | | | | | | | | | | | | | | |
| Phage- | M | V | K | W | — | T | — | P | — | — | — | N | F | E | 0.0557 | 1.0484 | 0.1211 | 2911 |
| 2595 | | | | | | | | | | | | | | | | | | |
| Phage- | M | F | K | W | — | T | — | S | — | — | — | E | R | V | 0.1237 | 1.0452 | 0.1759 | 2912 |
| 2596 | | | | | | | | | | | | | | | | | | |
| Phage- | V | H | K | W | — | T | — | A | — | — | — | E | V | H | 0.0585 | 1.045 | 0.0662 | 2913 |
| 2597 | | | | | | | | | | | | | | | | | | |
| Phage- | L | V | K | W | — | V | — | D | — | — | — | Q | E | T | 0.0867 | 1.0424 | 0.0671 | 2914 |
| 2598 | | | | | | | | | | | | | | | | | | |
| Phage- | L | S | K | W | — | I | — | D | — | — | — | I | S | S | 0.0609 | 1.0394 | 0.0618 | 2915 |
| 2599 | | | | | | | | | | | | | | | | | | |
| Phage- | S | V | T | W | — | I | — | P | — | — | — | E | L | S | 0.0637 | 1.0365 | 0.0673 | 2916 |
| 2600 | | | | | | | | | | | | | | | | | | |
| Phage- | — | E | K | W | — | M | — | S | — | — | — | E | W | N | 0.0568 | 1.0319 | 0.0609 | 2917 |
| 2601 | | | | | | | | | | | | | | | | | | |
| Phage- | E | T | K | — | — | K | — | M | — | — | — | — | L | L | 0.079 | 1.0306 | 0.1255 | 2918 |
| 2602 | | | | | | | | | | | | | | | | | | |
| Phage- | G | M | K | W | — | T | — | E | — | — | — | Q | I | V | 0.1583 | 1.0296 | 0.2804 | 2919 |
| 2603 | | | | | | | | | | | | | | | | | | |
| Phage- | V | W | Q | — | — | I | — | P | — | — | — | Q | F | E | 0.0807 | 1.0287 | 0.1128 | 2920 |
| 2604 | | | | | | | | | | | | | | | | | | |
| Phage- | V | M | K | W | — | T | — | S | — | — | — | E | H | I | 0.0564 | 1.028 | 0.0582 | 2921 |
| 2605 | | | | | | | | | | | | | | | | | | |
| Phage- | V | D | K | W | — | I | — | A | — | — | — | D | R | S | 0.0613 | 1.027 | 0.0604 | 2922 |
| 2606 | | | | | | | | | | | | | | | | | | |
| Phage- | Q | M | K | — | — | I | — | Q | — | — | — | V | D | H | 0.0557 | 1.0262 | 0.0553 | 2923 |
| 2607 | | | | | | | | | | | | | | | | | | |
| Phage- | L | H | K | — | — | I | — | E | — | — | — | E | Y | Y | 0.0541 | 1.0259 | 0.0595 | 2924 |
| 2608 | | | | | | | | | | | | | | | | | | |
| Phage- | M | V | K | W | — | T | — | A | — | — | — | T | L | E | 0.0598 | 1.0241 | 0.0801 | 2925 |
| 2609 | | | | | | | | | | | | | | | | | | |
| Phage- | S | S | M | — | — | I | — | P | — | — | — | E | Q | S | 0.0562 | 1.0235 | 0.0682 | 2926 |
| 2610 | | | | | | | | | | | | | | | | | | |
| Phage- | V | E | K | W | — | I | — | E | — | — | — | E | T | S | 0.0692 | 1.0219 | 0.0624 | 2927 |
| 2611 | | | | | | | | | | | | | | | | | | |
| Phage- | F | — | N | W | — | T | — | S | — | — | — | V | — | M | 0.068 | 1.0218 | 0.0676 | 2928 |
| 2612 | | | | | | | | | | | | | | | | | | |
| Phage- | M | Y | N | — | — | T | — | E | — | — | — | — | — | — | 0.0534 | 1.0185 | 0.0554 | 2929 |
| 2613 | | | | | | | | | | | | | | | | | | |
| Phage- | H | V | N | W | — | T | — | K | — | — | — | S | — | — | 0.0668 | 1.0151 | 0.0894 | 2930 |
| 2614 | | | | | | | | | | | | | | | | | | |
| Phage- | H | E | K | W | — | I | — | P | — | — | — | E | A | N | 0.0786 | 1.0143 | 0.0603 | 2931 |
| 2615 | | | | | | | | | | | | | | | | | | |
| Phage- | Q | D | N | W | — | I | — | R | — | — | — | E | A | M | 0.0694 | 1.0078 | 0.0626 | 2932 |
| 2616 | | | | | | | | | | | | | | | | | | |
| Phage- | Y | T | P | W | — | K | — | A | — | — | — | S | V | S | 0.066 | 1.007 | 0.0806 | 2933 |
| 2617 | | | | | | | | | | | | | | | | | | |
| Phage- | A | F | K | W | — | I | — | — | — | — | — | — | S | Q | 0.076 | 1.0063 | 0.0999 | 2934 |
| 2618 | | | | | | | | | | | | | | | | | | |
| Phage- | M | N | K | W | — | T | — | A | — | — | — | E | V | V | 0.067 | 0.9979 | 0.095 | 2935 |
| 2619 | | | | | | | | | | | | | | | | | | |
| Phage- | — | — | N | W | — | T | — | Q | — | — | — | D | — | — | 0.0802 | 0.9968 | 0.0631 | 2936 |
| 2620 | | | | | | | | | | | | | | | | | | |
| Phage- | V | P | K | W | — | T | — | D | — | — | — | Y | — | M | 0.0659 | 0.9963 | 0.0681 | 2937 |
| 2621 | | | | | | | | | | | | | | | | | | |
| Phage- | F | H | M | W | — | T | — | A | — | — | — | — | D | Q | 0.112 | 0.9961 | 0.1255 | 2938 |
| 2622 | | | | | | | | | | | | | | | | | | |
| Phage- | T | P | K | W | — | T | — | P | — | — | — | Q | H | G | 0.1288 | 0.9937 | 0.0728 | 2939 |
| 2623 | | | | | | | | | | | | | | | | | | |
| Phage- | M | T | Q | W | — | I | — | E | — | — | — | S | E | E | 0.0803 | 0.9897 | 0.0648 | 2940 |
| 2624 | | | | | | | | | | | | | | | | | | |
| Phage- | P | E | T | R | — | I | — | S | — | — | — | S | — | M | 0.07 | 0.9889 | 0.0912 | 2941 |
| 2625 | | | | | | | | | | | | | | | | | | |
| Phage- | V | E | M | — | — | K | — | A | — | — | — | — | N | Q | 0.063 | 0.9859 | 0.0829 | 2942 |
| 2626 | | | | | | | | | | | | | | | | | | |
| Phage- | W | E | K | W | — | T | — | S | G | — | — | D | A | R | 0.0635 | 0.9817 | 0.1059 | 2943 |
| 2627 | | | | | | | | | | | | | | | | | | |
| Phage- | L | N | N | W | — | T | — | L | — | — | — | Q | M | — | 0.1119 | 0.9789 | 0.2011 | 2944 |
| 2628 | | | | | | | | | | | | | | | | | | |
| Phage- | V | V | P | R | — | T | — | P | — | — | — | L | V | S | 0.0829 | 0.9764 | 0.0785 | 2945 |
| 2629 | | | | | | | | | | | | | | | | | | |
| Phage- | V | T | K | — | — | I | — | E | — | — | — | — | S | D | 0.0634 | 0.9745 | 0.0961 | 2946 |
| 2630 | | | | | | | | | | | | | | | | | | |
| Phage- | V | M | S | W | — | V | — | A | — | — | — | E | T | P | 0.0787 | 0.9727 | 0.0614 | 2947 |
| 2631 | | | | | | | | | | | | | | | | | | |
| Phage- | A | E | K | W | — | I | — | E | — | — | — | E | Q | L | 0.0694 | 0.9718 | 0.0717 | 2948 |
| 2632 | | | | | | | | | | | | | | | | | | |
| Phage- | T | H | H | W | — | I | — | A | — | — | — | E | Q | D | 0.082 | 0.9674 | 0.0809 | 2949 |
| 2633 | | | | | | | | | | | | | | | | | | |
| Phage- | T | D | K | — | — | R | — | P | — | — | — | E | L | M | 0.0759 | 0.9657 | 0.065 | 2950 |
| 2634 | | | | | | | | | | | | | | | | | | |
| Phage- | D | M | K | W | — | T | — | A | — | — | — | Q | S | S | 0.0575 | 0.9655 | 0.0701 | 2951 |
| 2635 | | | | | | | | | | | | | | | | | | |
| Phage- | D | D | R | W | — | I | — | T | — | — | — | F | L | D | 0.0652 | 0.9649 | 0.0733 | 2952 |
| 2636 | | | | | | | | | | | | | | | | | | |
| Phage- | T | V | K | W | — | T | — | P | — | — | — | E | E | S | 0.0643 | 0.9644 | 0.0943 | 2953 |
| 2637 | | | | | | | | | | | | | | | | | | |
| Phage- | W | — | K | W | — | T | — | V | — | — | — | Q | — | I | 0.1204 | 0.9609 | 0.1357 | 2954 |
| 2638 | | | | | | | | | | | | | | | | | | |
| Phage- | V | G | N | W | — | T | — | M | — | — | — | Q | A | — | 0.1058 | 0.9608 | 0.0864 | 2955 |
| 2639 | | | | | | | | | | | | | | | | | | |
| Phage- | V | P | K | W | — | T | — | E | — | — | — | I | I | H | 0.0867 | 0.9583 | 0.0705 | 2956 |
| 2640 | | | | | | | | | | | | | | | | | | |
| Phage- | A | E | K | — | — | I | — | D | — | — | — | D | V | Y | 0.0727 | 0.9568 | 0.0771 | 2957 |
| 2641 | | | | | | | | | | | | | | | | | | |
| Phage- | D | L | K | W | — | I | — | S | — | — | — | T | S | T | 0.06 | 0.9565 | 0.0977 | 2958 |
| 2642 | | | | | | | | | | | | | | | | | | |
| Phage- | V | E | K | — | — | K | — | A | — | — | — | N | — | Q | 0.0702 | 0.9557 | 0.0808 | 2959 |
| 2643 | | | | | | | | | | | | | | | | | | |
| Phage- | P | E | R | M | — | V | — | Q | — | — | — | T | V | T | 0.0793 | 0.9557 | 0.1074 | 2960 |
| 2644 | | | | | | | | | | | | | | | | | | |
| Phage- | F | P | K | W | — | T | — | E | — | — | — | S | L | T | 0.0524 | 0.955 | 0.0592 | 2961 |
| 2645 | | | | | | | | | | | | | | | | | | |
| Phage- | Q | P | K | W | — | T | — | D | — | — | — | T | — | I | 0.0777 | 0.9538 | 0.1046 | 2962 |
| 2646 | | | | | | | | | | | | | | | | | | |
| Phage- | L | V | K | W | — | I | — | D | — | — | — | S | L | S | 0.0683 | 0.9518 | 0.0865 | 2963 |
| 2647 | | | | | | | | | | | | | | | | | | |
| Phage- | E | Y | K | W | — | T | — | I | — | — | — | Q | — | N | 0.0595 | 0.9498 | 0.0626 | 2964 |
| 2648 | | | | | | | | | | | | | | | | | | |
| Phage- | T | W | K | W | — | T | — | A | — | — | — | S | V | M | 0.0706 | 0.9474 | 0.1132 | 2965 |
| 2649 | | | | | | | | | | | | | | | | | | |
| Phage- | D | V | K | W | — | I | — | P | — | — | — | P | S | — | 0.0705 | 0.9441 | 0.0771 | 2966 |
| 2650 | | | | | | | | | | | | | | | | | | |
| Phage- | L | Q | N | W | — | V | — | A | — | — | — | E | L | M | 0.0564 | 0.9409 | 0.068 | 2967 |
| 2651 | | | | | | | | | | | | | | | | | | |
| Phage- | S | N | H | — | — | T | — | P | — | — | — | Y | — | — | 0.0545 | 0.9394 | 0.0665 | 2968 |
| 2652 | | | | | | | | | | | | | | | | | | |
| Phage- | E | T | K | W | — | I | — | V | — | — | — | E | H | K | 0.0703 | 0.9341 | 0.1222 | 2969 |
| 2653 | | | | | | | | | | | | | | | | | | |
| Phage- | H | E | K | W | — | T | — | P | — | — | — | E | L | I | 0.0614 | 0.9288 | 0.0717 | 2970 |
| 2654 | | | | | | | | | | | | | | | | | | |
| Phage- | F | V | N | M | — | K | — | A | — | — | — | S | L | L | 0.0925 | 0.9232 | 0.0949 | 2971 |
| 2655 | | | | | | | | | | | | | | | | | | |
| Phage- | A | I | K | W | — | I | — | A | — | — | — | — | Q | A | 0.0574 | 0.92 | 0.0786 | 2972 |
| 2656 | | | | | | | | | | | | | | | | | | |
| Phage- | L | Q | K | W | — | I | — | A | — | — | — | S | S | T | 0.0623 | 0.9197 | 0.0717 | 2973 |
| 2657 | | | | | | | | | | | | | | | | | | |
| Phage- | M | P | K | W | — | T | — | Q | — | — | — | E | M | Y | 0.0641 | 0.9196 | 0.0641 | 2974 |
| 2658 | | | | | | | | | | | | | | | | | | |
| Phage- | G | E | H | W | — | T | — | A | — | — | — | S | V | M | 0.055 | 0.9188 | 0.6798 | 2975 |
| 2659 | | | | | | | | | | | | | | | | | | |
| Phage- | V | E | K | W | — | R | — | S | — | — | — | Q | Q | V | 0.0888 | 0.9139 | 0.09 | 2976 |
| 2660 | | | | | | | | | | | | | | | | | | |
| Phage- | A | P | K | W | — | T | — | S | — | — | — | Q | Q | V | 0.0855 | 0.9077 | 0.1215 | 2977 |
| 2661 | | | | | | | | | | | | | | | | | | |
| Phage- | D | Y | K | W | — | T | — | P | — | — | — | L | V | D | 0.1131 | 0.9037 | 0.083 | 2978 |
| 2662 | | | | | | | | | | | | | | | | | | |
| Phage- | V | V | K | W | — | T | — | A | — | — | — | S | L | N | 0.0712 | 0.9016 | 0.0846 | 2979 |
| 2663 | | | | | | | | | | | | | | | | | | |
| Phage- | A | P | H | — | — | I | — | K | — | — | — | N | P | S | 0.0768 | 0.899 | 0.1047 | 2980 |
| 2664 | | | | | | | | | | | | | | | | | | |
| Phage- | E | F | K | W | — | I | — | M | — | — | — | Q | A | N | 0.0777 | 0.8959 | 0.0753 | 2981 |
| 2665 | | | | | | | | | | | | | | | | | | |
| Phage- | P | P | K | W | — | I | — | A | — | — | — | — | — | M | 0.0648 | 0.8947 | 0.0794 | 2982 |
| 2666 | | | | | | | | | | | | | | | | | | |
| Phage- | V | T | N | W | — | I | — | E | — | — | — | N | — | N | 0.052 | 0.8942 | 0.0615 | 2983 |
| 2667 | | | | | | | | | | | | | | | | | | |
| Phage- | — | T | K | — | — | T | — | P | — | — | — | M | D | V | 0.0577 | 0.8862 | 0.0571 | 2984 |
| 2668 | | | | | | | | | | | | | | | | | | |
| Phage- | E | E | K | W | — | I | — | Y | — | — | — | E | A | V | 0.0653 | 0.8853 | 0.0691 | 2985 |
| 2669 | | | | | | | | | | | | | | | | | | |
| Phage- | A | G | K | — | — | I | — | — | — | — | — | E | E | Q | 0.0939 | 0.8844 | 0.0913 | 2986 |
| 2670 | | | | | | | | | | | | | | | | | | |
| Phage- | W | D | N | W | — | T | — | S | — | — | — | S | E | L | 0.0664 | 0.8832 | 0.0853 | 2987 |
| 2671 | | | | | | | | | | | | | | | | | | |
| Phage- | Q | E | K | W | — | I | — | E | — | — | — | S | V | R | 0.0607 | 0.879 | 0.0823 | 2988 |
| 2672 | | | | | | | | | | | | | | | | | | |
| Phage- | D | W | K | W | — | T | — | A | — | — | — | T | Q | T | 0.0976 | 0.8768 | 0.0698 | 2989 |
| 2673 | | | | | | | | | | | | | | | | | | |
| Phage- | L | D | M | — | — | I | — | A | — | — | — | E | P | R | 0.0716 | 0.8767 | 0.0925 | 2990 |
| 2674 | | | | | | | | | | | | | | | | | | |
| Phage- | E | H | K | W | — | I | — | D | — | — | — | W | E | D | 0.0595 | 0.8765 | 0.0562 | 2991 |
| 2675 | | | | | | | | | | | | | | | | | | |
| Phage- | — | — | K | W | — | T | — | P | — | — | — | — | D | N | 0.0562 | 0.8763 | 0.079 | 2992 |
| 2676 | | | | | | | | | | | | | | | | | | |
| Phage- | S | G | N | W | — | T | — | S | — | — | — | T | — | — | 0.059 | 0.8744 | 0.062 | 2993 |
| 2677 | | | | | | | | | | | | | | | | | | |
| Phage- | N | V | I | R | — | E | — | M | — | — | — | Y | P | S | 0.0577 | 0.8742 | 0.0918 | 2994 |
| 2678 | | | | | | | | | | | | | | | | | | |
| Phage- | A | G | H | W | — | I | — | M | — | — | — | Q | E | D | 0.0636 | 0.8741 | 0.0526 | 2995 |
| 2679 | | | | | | | | | | | | | | | | | | |
| Phage- | D | M | N | W | — | S | — | P | — | — | — | F | — | — | 0.0572 | 0.8733 | 0.0709 | 2996 |
| 2680 | | | | | | | | | | | | | | | | | | |
| Phage- | G | E | K | W | — | T | — | P | — | — | — | M | F | Q | 0.071 | 0.8612 | 0.0755 | 2997 |
| 2681 | | | | | | | | | | | | | | | | | | |
| Phage- | D | V | K | W | — | I | — | S | — | — | — | Q | D | V | 0.0945 | 0.859 | 0.0723 | 2998 |
| 2682 | | | | | | | | | | | | | | | | | | |
| Phage- | E | Y | A | W | — | I | — | E | — | — | — | G | W | I | 0.0859 | 0.8581 | 0.0663 | 2999 |
| 2683 | | | | | | | | | | | | | | | | | | |
| Phage- | S | E | A | W | — | I | — | S | — | — | — | R | — | D | 0.0736 | 0.8566 | 0.0558 | 3000 |
| 2684 | | | | | | | | | | | | | | | | | | |
| Phage- | P | M | K | — | — | S | — | P | — | — | — | F | Y | D | 0.0557 | 0.8499 | 0.0667 | 3001 |
| 2685 | | | | | | | | | | | | | | | | | | |
| Phage- | Q | N | I | R | — | V | — | P | — | — | — | E | N | L | 0.0661 | 0.8442 | 0.0867 | 3002 |
| 2686 | | | | | | | | | | | | | | | | | | |
| Phage- | V | H | Y | W | — | I | — | A | — | — | — | N | K | S | 0.1306 | 0.8406 | 0.1967 | 3003 |
| 2687 | | | | | | | | | | | | | | | | | | |
| Phage- | E | — | H | — | — | K | — | L | — | — | — | E | F | G | 0.0551 | 0.8403 | 0.0723 | 3004 |
| 2688 | | | | | | | | | | | | | | | | | | |
| Phage- | — | K | P | — | — | S | — | P | — | — | — | S | M | Y | 0.0708 | 0.8393 | 0.0721 | 3005 |
| 2689 | | | | | | | | | | | | | | | | | | |
| Phage- | L | V | K | W | — | T | — | T | — | — | — | V | — | M | 0.0841 | 0.8367 | 0.0655 | 3006 |
| 2690 | | | | | | | | | | | | | | | | | | |
| Phage- | — | V | K | W | — | T | — | P | — | — | — | E | R | P | 0.0679 | 0.834 | 0.0651 | 3007 |
| 2691 | | | | | | | | | | | | | | | | | | |
| Phage- | M | E | K | W | — | I | — | Q | — | — | — | E | Q | — | 0.0569 | 0.8336 | 0.0645 | 3008 |
| 2692 | | | | | | | | | | | | | | | | | | |
| Phage- | M | G | N | W | — | T | — | S | — | — | — | E | — | L | 0.0636 | 0.833 | 0.0732 | 3009 |
| 2693 | | | | | | | | | | | | | | | | | | |
| Phage- | G | E | K | W | — | T | — | L | — | — | — | E | W | Y | 0.0738 | 0.832 | 0.0871 | 3010 |
| 2694 | | | | | | | | | | | | | | | | | | |
| Phage- | H | W | R | M | — | V | — | T | — | — | — | H | L | D | 0.166 | 0.8298 | 0.1497 | 3011 |
| 2695 | | | | | | | | | | | | | | | | | | |
| Phage- | E | E | M | W | — | I | — | V | — | — | — | E | H | Q | 0.078 | 0.8287 | 0.0935 | 3012 |
| 2696 | | | | | | | | | | | | | | | | | | |
| Phage- | F | V | K | W | — | T | — | S | — | — | — | E | S | Q | 0.0656 | 0.8277 | 0.0785 | 3013 |
| 2697 | | | | | | | | | | | | | | | | | | |
| Phage- | D | N | K | M | — | I | — | V | — | — | — | E | R | — | 0.0524 | 0.8241 | 0.0685 | 3014 |
| 2698 | | | | | | | | | | | | | | | | | | |
| Phage- | G | S | H | W | — | E | — | L | — | — | — | S | A | — | 0.0751 | 0.8241 | 0.0853 | 3015 |
| 2699 | | | | | | | | | | | | | | | | | | |
| Phage- | — | E | K | W | — | T | — | P | — | — | — | E | S | S | 0.0666 | 0.8238 | 0.1044 | 3016 |
| 2700 | | | | | | | | | | | | | | | | | | |
| Phage- | D | N | M | — | — | R | — | S | — | — | — | E | — | L | 0.0522 | 0.8156 | 0.0762 | 3017 |
| 2701 | | | | | | | | | | | | | | | | | | |
| Phage- | Q | V | P | W | — | T | — | A | — | — | — | E | R | A | 0.0744 | 0.8135 | 0.1262 | 3018 |
| 2702 | | | | | | | | | | | | | | | | | | |
| Phage- | T | I | K | W | — | T | — | S | — | — | — | — | E | I | 0.0662 | 0.8106 | 0.1053 | 3019 |
| 2703 | | | | | | | | | | | | | | | | | | |
| Phage- | V | G | N | W | — | I | — | L | — | — | — | — | A | E | 0.0606 | 0.8104 | 0.0867 | 3020 |
| 2704 | | | | | | | | | | | | | | | | | | |
| Phage- | E | Q | M | W | — | T | — | A | — | — | — | T | — | — | 0.068 | 0.8099 | 0.0782 | 3021 |
| 2705 | | | | | | | | | | | | | | | | | | |
| Phage- | D | Y | K | W | — | T | — | P | — | — | — | Y | S | A | 0.0567 | 0.8043 | 0.0621 | 3022 |
| 2706 | | | | | | | | | | | | | | | | | | |
| Phage- | D | H | K | W | — | T | — | P | — | — | — | — | A | D | 0.0696 | 0.7974 | 0.0877 | 3023 |
| 2707 | | | | | | | | | | | | | | | | | | |
| Phage- | M | D | R | W | — | T | — | A | — | — | — | E | V | H | 0.0607 | 0.7941 | 0.0734 | 3024 |
| 2708 | | | | | | | | | | | | | | | | | | |
| Phage- | P | D | N | W | — | I | — | A | — | — | — | E | R | — | 0.0613 | 0.7929 | 0.0679 | 3025 |
| 2709 | | | | | | | | | | | | | | | | | | |
| Phage- | S | V | K | W | — | T | — | P | — | — | — | T | — | L | 0.0675 | 0.7921 | 0.0767 | 3026 |
| 2710 | | | | | | | | | | | | | | | | | | |
| Phage- | N | V | S | M | — | I | — | — | — | — | — | V | — | L | 0.0703 | 0.7915 | 0.0891 | 3027 |
| 2711 | | | | | | | | | | | | | | | | | | |
| Phage- | W | E | N | W | — | R | — | S | — | — | — | E | D | T | 0.0579 | 0.79 | 0.0568 | 3028 |
| 2712 | | | | | | | | | | | | | | | | | | |
| Phage- | N | T | H | W | — | T | — | S | — | — | — | S | A | W | 0.0807 | 0.7889 | 0.0816 | 3029 |
| 2713 | | | | | | | | | | | | | | | | | | |
| Phage- | H | E | K | W | — | V | — | S | — | — | — | D | D | W | 0.0613 | 0.7839 | 0.0669 | 3030 |
| 2714 | | | | | | | | | | | | | | | | | | |
| Phage- | V | Y | K | W | — | T | — | S | — | — | — | — | D | P | 0.0565 | 0.7791 | 0.0676 | 3031 |
| 2715 | | | | | | | | | | | | | | | | | | |
| Phage- | V | — | N | W | — | T | — | K | — | — | — | E | H | V | 0.0737 | 0.7774 | 0.0631 | 3032 |
| 2716 | | | | | | | | | | | | | | | | | | |
| Phage- | E | Y | T | W | — | K | — | A | — | — | — | D | — | V | 0.103 | 0.7745 | 0.0742 | 3033 |
| 2717 | | | | | | | | | | | | | | | | | | |
| Phage- | T | Q | K | W | — | I | — | A | — | — | — | — | E | R | 0.1072 | 0.7743 | 0.0702 | 3034 |
| 2718 | | | | | | | | | | | | | | | | | | |
| Phage- | D | M | H | W | — | T | — | Q | — | — | — | — | P | L | 0.0499 | 0.7712 | 0.0856 | 3035 |
| 2719 | | | | | | | | | | | | | | | | | | |
| Phage- | D | W | K | W | — | T | — | S | — | — | — | — | Q | K | 0.0587 | 0.769 | 0.0639 | 3036 |
| 2720 | | | | | | | | | | | | | | | | | | |
| Phage- | N | V | K | W | — | V | — | H | — | — | — | E | V | S | 0.0743 | 0.7655 | 0.0649 | 3037 |
| 2721 | | | | | | | | | | | | | | | | | | |
| Phage- | D | Q | K | W | — | I | — | L | — | — | — | E | T | R | 0.0785 | 0.7625 | 0.0727 | 3038 |
| 2722 | | | | | | | | | | | | | | | | | | |
| Phage- | D | Y | K | W | — | T | — | M | — | — | — | E | V | V | 0.0628 | 0.7527 | 0.0761 | 3039 |
| 2723 | | | | | | | | | | | | | | | | | | |
| Phage- | E | W | K | W | — | T | — | P | — | — | — | T | T | N | 0.0742 | 0.7517 | 0.0835 | 3040 |
| 2724 | | | | | | | | | | | | | | | | | | |
| Phage- | M | E | K | M | — | T | — | Q | — | — | — | D | M | W | 0.062 | 0.7492 | 0.0732 | 3041 |
| 2725 | | | | | | | | | | | | | | | | | | |
| Phage- | — | N | K | W | — | T | — | A | — | — | — | D | L | S | 0.1103 | 0.7468 | 0.0937 | 3042 |
| 2726 | | | | | | | | | | | | | | | | | | |
| Phage- | W | Q | K | W | — | T | — | M | — | — | — | E | A | T | 0.0759 | 0.7444 | 0.0785 | 3043 |
| 2727 | | | | | | | | | | | | | | | | | | |
| Phage- | V | N | P | S | — | T | — | S | — | — | — | L | — | L | 0.0615 | 0.7393 | 0.0793 | 3044 |
| 2728 | | | | | | | | | | | | | | | | | | |
| Phage- | L | Y | N | R | — | T | — | A | — | — | — | T | — | — | 0.0591 | 0.7384 | 0.0657 | 3045 |
| 2729 | | | | | | | | | | | | | | | | | | |
| Phage- | E | M | K | W | — | K | — | E | — | — | — | P | W | M | 0.0667 | 0.7332 | 0.0688 | 3046 |
| 2730 | | | | | | | | | | | | | | | | | | |
| Phage- | V | S | N | W | — | K | — | A | — | — | — | G | T | T | 0.0657 | 0.7332 | 0.079 | 3047 |
| 2731 | | | | | | | | | | | | | | | | | | |
| Phage- | W | E | K | W | — | T | — | P | — | — | — | H | D | M | 0.0826 | 0.7327 | 0.0669 | 3048 |
| 2732 | | | | | | | | | | | | | | | | | | |
| Phage- | D | I | K | W | — | T | — | S | — | — | — | P | A | K | 0.1088 | 0.7293 | 0.0862 | 3049 |
| 2733 | | | | | | | | | | | | | | | | | | |
| Phage- | Q | M | P | — | — | I | — | Q | — | — | — | T | N | N | 0.0586 | 0.7233 | 0.0562 | 3050 |
| 2734 | | | | | | | | | | | | | | | | | | |
| Phage- | H | G | K | — | — | T | — | S | — | — | — | N | — | — | 0.062 | 0.7208 | 0.0619 | 3051 |
| 2735 | | | | | | | | | | | | | | | | | | |
| Phage- | — | V | K | W | — | T | — | A | — | — | — | D | Q | Q | 0.0688 | 0.72 | 0.0809 | 3052 |
| 2736 | | | | | | | | | | | | | | | | | | |
| Phage- | T | N | N | W | — | I | — | S | — | — | — | D | I | R | 0.057 | 0.7178 | 0.0629 | 3053 |
| 2737 | | | | | | | | | | | | | | | | | | |
| Phage- | W | E | K | — | — | R | — | A | — | — | — | — | A | Y | 0.0674 | 0.7152 | 0.0754 | 3054 |
| 2738 | | | | | | | | | | | | | | | | | | |
| Phage- | F | E | N | W | — | T | — | Y | — | — | — | S | — | M | 0.0593 | 0.7122 | 0.0884 | 3055 |
| 2739 | | | | | | | | | | | | | | | | | | |
| Phage- | G | N | S | W | — | T | — | Q | — | — | — | T | — | — | 0.0584 | 0.7119 | 0.0596 | 3056 |
| 2740 | | | | | | | | | | | | | | | | | | |
| Phage- | M | S | K | W | — | V | — | T | — | — | — | Q | W | Q | 0.0919 | 0.7106 | 0.0979 | 3057 |
| 2741 | | | | | | | | | | | | | | | | | | |
| Phage- | D | I | K | R | — | T | — | A | — | — | — | S | W | M | 0.0649 | 0.7093 | 0.0807 | 3058 |
| 2742 | | | | | | | | | | | | | | | | | | |
| Phage- | — | M | K | W | — | T | — | T | — | — | — | E | M | G | 0.0847 | 0.7079 | 0.1221 | 3059 |
| 2743 | | | | | | | | | | | | | | | | | | |
| Phage- | F | D | K | W | — | T | — | P | — | — | — | K | V | — | 0.0804 | 0.7076 | 0.1432 | 3060 |
| 2744 | | | | | | | | | | | | | | | | | | |
| Phage- | T | N | K | — | — | I | — | T | — | — | — | S | — | — | 0.0603 | 0.7026 | 0.0715 | 3061 |
| 2745 | | | | | | | | | | | | | | | | | | |
| Phage- | V | V | K | M | — | K | — | L | — | — | — | G | I | S | 0.0591 | 0.7012 | 0.0564 | 3062 |
| 2746 | | | | | | | | | | | | | | | | | | |
| Phage- | S | E | K | W | — | T | — | A | — | — | — | G | D | M | 0.0946 | 0.7001 | 0.0842 | 3063 |
| 2747 | | | | | | | | | | | | | | | | | | |
| Phage- | D | W | K | W | — | I | — | A | — | — | — | S | Q | D | 0.0637 | 0.6974 | 0.0645 | 3064 |
| 2748 | | | | | | | | | | | | | | | | | | |
| Phage- | G | N | L | W | — | S | — | P | — | — | — | F | — | M | 0.0547 | 0.6957 | 0.0732 | 3065 |
| 2749 | | | | | | | | | | | | | | | | | | |
| Phage- | D | V | K | W | — | K | — | R | — | — | — | N | R | D | 0.0949 | 0.6949 | 0.0701 | 3066 |
| 2750 | | | | | | | | | | | | | | | | | | |
| Phage- | E | D | I | W | — | I | — | S | — | — | — | G | V | I | 0.0717 | 0.6944 | 0.0663 | 3067 |
| 2751 | | | | | | | | | | | | | | | | | | |
| Phage- | E | T | S | W | — | R | — | T | — | — | — | E | — | M | 0.0665 | 0.6927 | 0.0587 | 3068 |
| 2752 | | | | | | | | | | | | | | | | | | |
| Phage- | A | N | H | W | — | I | — | S | — | — | — | T | E | V | 0.0621 | 0.6923 | 0.1616 | 3069 |
| 2753 | | | | | | | | | | | | | | | | | | |
| Phage- | A | P | Q | W | — | I | — | P | — | — | — | H | D | Q | 0.0564 | 0.6911 | 0.0708 | 3070 |
| 2754 | | | | | | | | | | | | | | | | | | |
| Phage- | D | P | K | W | — | T | — | S | — | — | — | D | W | R | 0.0773 | 0.6897 | 0.0821 | 3071 |
| 2755 | | | | | | | | | | | | | | | | | | |
| Phage- | A | H | N | W | — | T | — | A | — | — | — | M | W | D | 0.0621 | 0.6819 | 0.0753 | 3072 |
| 2756 | | | | | | | | | | | | | | | | | | |
| Phage- | G | E | I | R | — | S | — | A | — | — | — | D | — | — | 0.0672 | 0.6816 | 0.1043 | 3073 |
| 2757 | | | | | | | | | | | | | | | | | | |
| Phage- | M | V | H | W | — | T | — | S | — | — | — | S | A | L | 0.0986 | 0.6773 | 0.1002 | 3074 |
| 2758 | | | | | | | | | | | | | | | | | | |
| Phage- | M | Q | K | M | — | I | — | T | — | — | — | D | — | L | 0.0779 | 0.6735 | 0.0817 | 3075 |
| 2759 | | | | | | | | | | | | | | | | | | |
| Phage- | P | V | K | M | — | T | — | A | — | — | — | S | E | R | 0.1071 | 0.6698 | 0.0866 | 3076 |
| 2760 | | | | | | | | | | | | | | | | | | |
| Phage- | P | W | K | W | — | T | — | V | — | — | — | Q | — | V | 0.08 | 0.6694 | 0.0937 | 3077 |
| 2761 | | | | | | | | | | | | | | | | | | |
| Phage- | W | I | N | W | — | T | — | Q | — | — | — | — | D | L | 0.1041 | 0.6622 | 0.1295 | 3078 |
| 2762 | | | | | | | | | | | | | | | | | | |
| Phage- | F | E | K | W | — | T | — | T | — | — | — | W | P | Y | 0.0661 | 0.6614 | 0.0829 | 3079 |
| 2763 | | | | | | | | | | | | | | | | | | |
| Phage- | E | N | H | W | — | R | — | M | — | — | — | E | T | I | 0.0677 | 0.6597 | 0.0683 | 3080 |
| 2764 | | | | | | | | | | | | | | | | | | |
| Phage- | H | — | I | W | — | I | — | E | — | — | — | — | F | N | 0.0948 | 0.6576 | 0.1241 | 3081 |
| 2765 | | | | | | | | | | | | | | | | | | |
| Phage- | V | E | K | W | — | T | — | A | — | — | — | S | T | S | 0.0607 | 0.6572 | 0.065 | 3082 |
| 2766 | | | | | | | | | | | | | | | | | | |
| Phage- | D | V | T | W | — | I | — | P | — | — | — | M | Y | N | 0.0855 | 0.6548 | 0.0814 | 3083 |
| 2767 | | | | | | | | | | | | | | | | | | |
| Phage- | A | E | N | W | — | T | — | R | — | — | — | E | — | I | 0.0932 | 0.6547 | 0.2013 | 3084 |
| 2768 | | | | | | | | | | | | | | | | | | |
| Phage- | S | Y | K | W | — | T | — | D | — | — | — | V | A | V | 0.0565 | 0.6513 | 0.0709 | 3085 |
| 2769 | | | | | | | | | | | | | | | | | | |
| Phage- | Q | I | K | W | — | I | — | M | — | — | — | E | — | E | 0.0566 | 0.6508 | 0.0554 | 3086 |
| 2770 | | | | | | | | | | | | | | | | | | |
| Phage- | Q | M | H | W | — | M | — | P | — | — | — | E | — | I | 0.0631 | 0.6502 | 0.0705 | 3087 |
| 2771 | | | | | | | | | | | | | | | | | | |
| Phage- | V | Q | A | W | — | V | — | Q | — | — | — | N | Q | — | 0.0534 | 0.6459 | 0.0631 | 3088 |
| 2772 | | | | | | | | | | | | | | | | | | |
| Phage- | V | P | K | W | — | T | — | S | — | — | — | — | S | N | 0.0502 | 0.6433 | 0.0608 | 3089 |
| 2773 | | | | | | | | | | | | | | | | | | |
| Phage- | S | M | K | M | — | T | — | P | — | — | — | E | F | A | 0.0621 | 0.6407 | 0.0631 | 3090 |
| 2774 | | | | | | | | | | | | | | | | | | |
| Phage- | D | L | K | W | — | V | — | T | — | — | — | N | W | S | 0.0699 | 0.6389 | 0.0692 | 3091 |
| 2775 | | | | | | | | | | | | | | | | | | |
| Phage- | G | P | K | W | — | T | — | T | — | — | — | E | F | S | 0.0747 | 0.6384 | 0.0754 | 3092 |
| 2776 | | | | | | | | | | | | | | | | | | |
| Phage- | G | S | N | W | — | K | — | A | — | — | — | S | V | A | 0.0497 | 0.6342 | 0.0623 | 3093 |
| 2777 | | | | | | | | | | | | | | | | | | |
| Phage- | Q | D | K | W | — | T | — | T | — | — | — | E | — | L | 0.0631 | 0.6308 | 0.0777 | 3094 |
| 2778 | | | | | | | | | | | | | | | | | | |
| Phage- | D | Q | N | W | — | I | — | H | — | — | — | V | — | I | 0.0809 | 0.6249 | 0.0883 | 3095 |
| 2779 | | | | | | | | | | | | | | | | | | |
| Phage- | V | L | N | W | — | T | — | P | — | — | — | S | — | I | 0.0532 | 0.624 | 0.0732 | 3096 |
| 2780 | | | | | | | | | | | | | | | | | | |
| Phage- | G | W | N | W | — | V | — | Q | — | — | — | T | T | S | 0.1021 | 0.623 | 0.1207 | 3097 |
| 2781 | | | | | | | | | | | | | | | | | | |
| Phage- | Q | D | K | M | — | R | — | D | — | — | — | M | N | Q | 0.0612 | 0.6207 | 0.0706 | 3098 |
| 2782 | | | | | | | | | | | | | | | | | | |
| Phage- | V | H | K | M | — | T | — | P | — | — | — | V | T | Q | 0.0672 | 0.6207 | 0.0908 | 3099 |
| 2783 | | | | | | | | | | | | | | | | | | |
| Phage- | D | M | K | — | — | R | — | A | — | — | — | M | H | N | 0.0544 | 0.6206 | 0.0536 | 3100 |
| 2784 | | | | | | | | | | | | | | | | | | |
| Phage- | D | V | K | W | — | T | — | P | — | — | — | L | S | D | 0.0854 | 0.6127 | 0.1003 | 3101 |
| 2785 | | | | | | | | | | | | | | | | | | |
| Phage- | E | E | N | W | — | I | — | V | — | — | — | E | R | L | 0.0796 | 0.6071 | 0.0721 | 3102 |
| 2786 | | | | | | | | | | | | | | | | | | |
| Phage- | L | W | K | S | — | T | — | E | — | — | — | — | E | W | 0.0888 | 0.6061 | 0.0991 | 3103 |
| 2787 | | | | | | | | | | | | | | | | | | |
| Phage- | S | V | K | W | — | T | — | S | — | — | — | T | E | W | 0.0817 | 0.6006 | 0.1044 | 3104 |
| 2788 | | | | | | | | | | | | | | | | | | |
| Phage- | V | G | K | W | — | T | — | E | — | — | — | S | — | — | 0.058 | 0.5995 | 0.0562 | 3105 |
| 2789 | | | | | | | | | | | | | | | | | | |
| Phage- | V | M | K | W | — | T | — | S | — | — | — | — | E | A | 0.067 | 0.5993 | 0.0742 | 3106 |
| 2790 | | | | | | | | | | | | | | | | | | |
| Phage- | W | E | K | W | — | T | — | S | — | — | — | S | — | I | 0.0564 | 0.5975 | 0.0859 | 3107 |
| 2791 | | | | | | | | | | | | | | | | | | |
| Phage- | T | Q | H | W | — | T | — | I | — | — | — | E | — | L | 0.0865 | 0.597 | 0.0819 | 3108 |
| 2792 | | | | | | | | | | | | | | | | | | |
| Phage- | V | T | N | W | — | T | — | P | — | — | — | E | M | L | 0.0676 | 0.5964 | 0.2135 | 3109 |
| 2793 | | | | | | | | | | | | | | | | | | |
| Phage- | T | E | K | W | — | I | — | Q | — | — | — | V | R | T | 0.056 | 0.5919 | 0.0656 | 3110 |
| 2794 | | | | | | | | | | | | | | | | | | |
| Phage- | F | E | K | W | — | T | — | Q | — | — | — | N | S | M | 0.0569 | 0.59 | 0.0699 | 3111 |
| 2795 | | | | | | | | | | | | | | | | | | |
| Phage- | L | N | N | W | — | T | — | A | — | — | — | V | — | Y | 0.0697 | 0.5891 | 0.084 | 3112 |
| 2796 | | | | | | | | | | | | | | | | | | |
| Phage- | E | R | I | W | — | T | — | K | — | — | — | E | T | — | 0.0696 | 0.5887 | 0.0657 | 3113 |
| 2797 | | | | | | | | | | | | | | | | | | |
| Phage- | H | F | K | W | — | I | — | P | — | — | — | L | E | I | 0.0798 | 0.5882 | 0.2982 | 3114 |
| 2798 | | | | | | | | | | | | | | | | | | |
| Phage- | E | S | K | W | — | V | — | T | — | — | — | — | E | N | 0.0516 | 0.5828 | 0.0578 | 3115 |
| 2799 | | | | | | | | | | | | | | | | | | |
| Phage- | D | Y | K | W | — | T | — | P | — | — | — | D | S | R | 0.0795 | 0.58 | 0.0697 | 3116 |
| 2800 | | | | | | | | | | | | | | | | | | |
| Phage- | S | D | N | W | — | K | — | E | — | — | — | V | — | H | 0.0738 | 0.5775 | 0.0873 | 3117 |
| 2801 | | | | | | | | | | | | | | | | | | |
| Phage- | T | P | K | W | — | T | — | A | — | — | — | V | V | S | 0.0829 | 0.5751 | 0.077 | 3118 |
| 2802 | | | | | | | | | | | | | | | | | | |
| Phage- | V | K | H | V | — | M | — | E | — | — | — | Y | W | S | 0.0652 | 0.5704 | 0.0724 | 3119 |
| 2803 | | | | | | | | | | | | | | | | | | |
| Phage- | E | G | K | W | — | T | — | T | — | — | — | Q | — | — | 0.0669 | 0.5703 | 0.0945 | 3120 |
| 2804 | | | | | | | | | | | | | | | | | | |
| Phage- | M | M | H | W | — | T | — | P | — | — | — | E | F | S | 0.0597 | 0.5676 | 0.0626 | 3121 |
| 2805 | | | | | | | | | | | | | | | | | | |
| Phage- | G | — | T | W | — | T | — | T | — | — | — | T | — | — | 0.0609 | 0.5658 | 0.117 | 3122 |
| 2806 | | | | | | | | | | | | | | | | | | |
| Phage- | W | E | N | W | — | I | — | Q | — | — | — | G | T | A | 0.1629 | 0.565 | 0.1415 | 3123 |
| 2807 | | | | | | | | | | | | | | | | | | |
| Phage- | M | E | K | W | — | T | — | R | — | — | — | V | — | M | 0.0767 | 0.5649 | 0.0734 | 3124 |
| 2808 | | | | | | | | | | | | | | | | | | |
| Phage- | V | T | K | — | — | I | — | E | — | — | — | V | I | Q | 0.0543 | 0.5625 | 0.0595 | 3125 |
| 2809 | | | | | | | | | | | | | | | | | | |
| Phage- | M | D | I | W | — | K | — | R | — | — | — | E | E | Y | 0.0644 | 0.5616 | 0.0676 | 3126 |
| 2810 | | | | | | | | | | | | | | | | | | |
| Phage- | V | P | K | W | — | T | — | S | — | — | — | E | F | T | 0.0593 | 0.5601 | 0.0648 | 3127 |
| 2811 | | | | | | | | | | | | | | | | | | |
| Phage- | V | G | P | W | — | I | — | D | — | — | — | D | V | V | 0.0572 | 0.5538 | 0.0755 | 3128 |
| 2812 | | | | | | | | | | | | | | | | | | |
| Phage- | F | S | K | — | — | T | — | P | — | — | — | S | V | S | 0.0586 | 0.5429 | 0.2757 | 3129 |
| 2813 | | | | | | | | | | | | | | | | | | |
| Phage- | P | M | K | W | — | T | — | P | — | — | — | S | Y | S | 0.0793 | 0.5383 | 0.063 | 3130 |
| 2814 | | | | | | | | | | | | | | | | | | |
| Phage- | — | D | K | W | — | V | — | A | — | — | — | S | P | A | 0.0608 | 0.5379 | 0.06 | 3131 |
| 2815 | | | | | | | | | | | | | | | | | | |
| Phage- | G | D | K | W | — | K | — | S | — | — | — | D | F | — | 0.0602 | 0.5263 | 0.072 | 3132 |
| 2816 | | | | | | | | | | | | | | | | | | |
| Phage- | E | — | K | W | — | T | — | P | — | — | — | S | L | V | 0.0615 | 0.5249 | 0.0546 | 3133 |
| 2817 | | | | | | | | | | | | | | | | | | |
| Phage- | S | V | H | M | — | K | — | P | — | — | — | Q | S | M | 0.0587 | 0.5094 | 0.077 | 3134 |
| 2818 | | | | | | | | | | | | | | | | | | |
| Phage- | — | D | K | W | — | K | — | V | — | — | — | — | D | N | 0.06 | 0.5005 | 0.0659 | 3135 |
| 2819 | | | | | | | | | | | | | | | | | | |
| Phage- | F | E | K | W | — | T | — | L | — | — | — | — | D | S | 0.0702 | 0.4999 | 0.0612 | 3136 |
| 2820 | | | | | | | | | | | | | | | | | | |
| Phage- | W | D | N | W | — | T | — | S | — | — | — | S | T | A | 0.0795 | 0.4885 | 0.0814 | 3137 |
| 2821 | | | | | | | | | | | | | | | | | | |
| Phage- | D | I | R | W | — | T | — | P | — | — | — | M | H | L | 0.0539 | 0.4861 | 0.2582 | 3138 |
| 2822 | | | | | | | | | | | | | | | | | | |
| Phage- | G | V | N | W | — | V | — | P | — | — | — | L | N | D | 0.0721 | 0.4839 | 0.0879 | 3139 |
| 2823 | | | | | | | | | | | | | | | | | | |
| Phage- | G | L | P | W | — | V | — | Q | — | — | — | V | V | S | 0.0619 | 0.482 | 0.0721 | 3140 |
| 2824 | | | | | | | | | | | | | | | | | | |
| Phage- | Y | H | K | W | — | T | — | E | — | — | — | V | F | D | 0.073 | 0.4741 | 0.0666 | 3141 |
| 2825 | | | | | | | | | | | | | | | | | | |
| Phage- | S | S | N | W | — | T | — | A | — | — | — | V | T | S | 0.0787 | 0.4702 | 0.0627 | 3142 |
| 2826 | | | | | | | | | | | | | | | | | | |
| Phage- | L | E | K | W | — | I | — | S | Q | L | — | Q | L | D | 0.0876 | 0.4642 | 0.0743 | 3143 |
| 2827 | | | | | | | | | | | | | | | | | | |
| Phage- | G | E | K | W | — | S | — | A | — | — | — | E | W | L | 0.0668 | 0.4586 | 0.0817 | 3144 |
| 2828 | | | | | | | | | | | | | | | | | | |
| Phage- | D | V | K | W | — | T | — | R | — | — | — | E | L | S | 0.0707 | 0.4573 | 0.0913 | 3145 |
| 2829 | | | | | | | | | | | | | | | | | | |
| Phage- | Q | — | I | W | — | I | — | D | — | — | — | V | Q | N | 0.0611 | 0.4539 | 0.1371 | 3146 |
| 2830 | | | | | | | | | | | | | | | | | | |
| Phage- | A | F | P | S | — | I | — | E | — | — | — | S | — | M | 0.0565 | 0.4498 | 0.1488 | 3147 |
| 2831 | | | | | | | | | | | | | | | | | | |
| Phage- | E | F | K | W | — | S | — | A | Q | L | — | V | N | G | 0.0715 | 0.4474 | 0.0526 | 3148 |
| 2832 | | | | | | | | | | | | | | | | | | |
| Phage- | E | V | K | W | — | T | — | Y | — | — | — | I | D | T | 0.058 | 0.4433 | 0.072 | 3149 |
| 2833 | | | | | | | | | | | | | | | | | | |
| Phage- | N | T | N | W | — | T | — | P | — | — | — | E | — | L | 0.0662 | 0.4423 | 0.0664 | 3150 |
| 2834 | | | | | | | | | | | | | | | | | | |
| Phage- | V | — | K | — | — | T | — | A | — | — | — | S | A | M | 0.0643 | 0.4309 | 0.0638 | 3151 |
| 2835 | | | | | | | | | | | | | | | | | | |
| Phage- | D | W | K | — | — | K | — | T | — | — | — | E | S | L | 0.0582 | 0.4304 | 0.0664 | 3152 |
| 2836 | | | | | | | | | | | | | | | | | | |
| Phage- | V | D | R | W | — | T | — | P | — | — | — | D | W | Q | 0.0583 | 0.4289 | 0.0626 | 3153 |
| 2837 | | | | | | | | | | | | | | | | | | |
| Phage- | H | P | K | — | — | T | — | E | — | — | — | V | I | Q | 0.0666 | 0.4237 | 0.1045 | 3154 |
| 2838 | | | | | | | | | | | | | | | | | | |
| Phage- | D | S | H | W | — | M | — | V | — | — | — | M | — | L | 0.0737 | 0.4232 | 0.1564 | 3155 |
| 2839 | | | | | | | | | | | | | | | | | | |
| Phage- | E | P | Q | W | — | I | — | K | — | — | — | D | H | M | 0.0612 | 0.4231 | 0.0608 | 3156 |
| 2840 | | | | | | | | | | | | | | | | | | |
| Phage- | E | S | I | R | — | T | — | A | — | — | — | V | E | M | 0.0625 | 0.4211 | 0.0725 | 3157 |
| 2841 | | | | | | | | | | | | | | | | | | |
| Phage- | P | E | — | M | — | R | — | V | — | — | — | L | — | — | 0.0609 | 2.5071 | 0.1122 | 3158 |
| 2842 | | | | | | | | | | | | | | | | | | |
| Phage- | M | L | P | I | — | K | — | D | — | — | — | — | — | — | 0.0724 | 2.4889 | 0.0942 | 3159 |
| 2843 | | | | | | | | | | | | | | | | | | |
| Phage- | V | V | A | M | — | K | — | A | — | — | — | — | — | L | 0.0591 | 2.4826 | 0.0731 | 3160 |
| 2844 | | | | | | | | | | | | | | | | | | |
| Phage- | W | D | M | — | — | R | — | A | — | — | — | E | — | — | 0.0711 | 2.4806 | 0.1572 | 3161 |
| 2845 | | | | | | | | | | | | | | | | | | |
| Phage- | W | E | — | R | — | V | — | E | — | — | — | V | — | — | 0.0615 | 2.4771 | 0.1189 | 3162 |
| 2846 | | | | | | | | | | | | | | | | | | |
| Phage- | W | M | P | R | — | V | — | E | — | — | — | — | — | — | 0.1667 | 2.4762 | 0.3338 | 3163 |
| 2847 | | | | | | | | | | | | | | | | | | |
| Phage- | V | M | T | V | — | K | — | A | — | — | — | T | — | — | 0.1338 | 2.4678 | 0.2049 | 3164 |
| 2848 | | | | | | | | | | | | | | | | | | |
| Phage- | V | F | P | I | — | K | — | S | — | — | — | V | — | M | 0.0918 | 2.4639 | 0.1743 | 3165 |
| 2849 | | | | | | | | | | | | | | | | | | |
| Phage- | V | N | I | R | — | E | — | A | — | — | — | M | — | — | 0.0727 | 2.4632 | 0.1081 | 3166 |
| 2850 | | | | | | | | | | | | | | | | | | |
| Phage- | D | M | R | — | — | V | — | E | — | — | — | L | — | L | 0.0889 | 2.463 | 0.0789 | 3167 |
| 2851 | | | | | | | | | | | | | | | | | | |
| Phage- | V | M | P | — | — | K | — | A | — | — | — | G | — | — | 0.0869 | 2.458 | 0.1338 | 3168 |
| 2852 | | | | | | | | | | | | | | | | | | |
| Phage- | M | L | P | — | — | V | — | A | — | — | — | S | — | — | 0.061 | 2.4448 | 0.1199 | 3169 |
| 2853 | | | | | | | | | | | | | | | | | | |
| Phage- | V | H | I | M | — | K | — | A | — | | — | L | — | — | 0.0917 | 2.4446 | 0.2464 | 3170 |
| 2854 | | | | | | | | | | | | | | | | | | |
| Phage- | W | E | M | K | — | I | — | E | — | — | — | R | — | — | 0.0794 | 2.4398 | 0.1393 | 3171 |
| 2855 | | | | | | | | | | | | | | | | | | |
| Phage- | V | Q | R | M | — | E | — | S | — | — | — | F | — | — | 0.0631 | 2.4395 | 0.0783 | 3172 |
| 2856 | | | | | | | | | | | | | | | | | | |
| Phage- | L | E | R | A | — | V | — | S | — | — | — | T | — | — | 0.0649 | 2.4377 | 0.0938 | 3173 |
| 2857 | | | | | | | | | | | | | | | | | | |
| Phage- | T | Q | A | M | — | K | — | A | — | — | — | Q | — | — | 0.0585 | 2.4355 | 0.0783 | 3174 |
| 2858 | | | | | | | | | | | | | | | | | | |
| Phage- | W | E | F | V | — | K | — | E | — | — | — | T | — | — | 0.0551 | 2.4284 | 0.1024 | 3175 |
| 2859 | | | | | | | | | | | | | | | | | | |
| Phage- | E | W | I | — | — | K | — | A | — | — | — | D | — | L | 0.051 | 2.4278 | 0.0639 | 3176 |
| 2860 | | | | | | | | | | | | | | | | | | |
| Phage- | W | D | R | — | — | I | — | A | — | — | — | S | — | — | 0.0903 | 2.4265 | 0.197 | 3177 |
| 2861 | | | | | | | | | | | | | | | | | | |
| Phage- | T | G | A | Y | — | V | — | Q | — | — | — | S | — | — | 0.0638 | 2.4259 | 0.1116 | 3178 |
| 2862 | | | | | | | | | | | | | | | | | | |
| Phage- | F | N | — | M | — | R | — | A | — | — | — | D | F | L | 0.1545 | 2.4244 | 0.1578 | 3179 |
| 2863 | | | | | | | | | | | | | | | | | | |
| Phage- | — | E | T | W | — | V | — | R | — | — | — | E | — | — | 0.0693 | 2.4154 | 0.2345 | 3180 |
| 2864 | | | | | | | | | | | | | | | | | | |
| Phage- | M | S | P | — | — | K | — | V | — | — | — | — | — | — | 0.0586 | 2.4042 | 0.0828 | 3181 |
| 2865 | | | | | | | | | | | | | | | | | | |
| Phage- | T | S | P | A | — | V | — | S | — | — | — | V | — | — | 0.0552 | 2.4035 | 0.071 | 3182 |
| 2866 | | | | | | | | | | | | | | | | | | |
| Phage- | V | L | P | M | — | S | — | P | — | — | — | I | — | L | 0.0631 | 2.4011 | 0.0912 | 3183 |
| 2867 | | | | | | | | | | | | | | | | | | |
| Phage- | E | P | I | R | — | V | — | Q | — | — | — | V | — | — | 0.0882 | 2.3995 | 0.0918 | 3184 |
| 2868 | | | | | | | | | | | | | | | | | | |
| Phage- | W | P | Q | — | — | V | — | M | — | — | — | — | — | L | 0.1456 | 2.3994 | 0.1482 | 3185 |
| 2869 | | | | | | | | | | | | | | | | | | |
| Phage- | S | E | F | W | — | V | — | A | — | — | — | E | — | — | 0.0542 | 2.395 | 0.0842 | 3186 |
| 2870 | | | | | | | | | | | | | | | | | | |
| Phage- | L | E | R | V | — | K | — | L | — | — | — | T | — | L | 0.0929 | 2.3948 | 0.1339 | 3187 |
| 2871 | | | | | | | | | | | | | | | | | | |
| Phage- | — | D | I | — | — | R | — | L | — | — | — | N | — | M | 0.0587 | 2.3915 | 0.1133 | 3188 |
| 2872 | | | | | | | | | | | | | | | | | | |
| Phage- | — | I | R | H | — | V | — | M | — | — | — | Q | — | — | 0.1005 | 2.3909 | 0.2237 | 3189 |
| 2873 | | | | | | | | | | | | | | | | | | |
| Phage- | E | T | L | R | — | E | — | M | — | — | — | M | — | — | 0.0988 | 2.3862 | 0.0975 | 3190 |
| 2874 | | | | | | | | | | | | | | | | | | |
| Phage- | S | M | P | R | — | V | — | V | — | — | — | E | — | — | 0.0623 | 2.3851 | 0.1068 | 3191 |
| 2875 | | | | | | | | | | | | | | | | | | |
| Phage- | D | D | I | R | — | K | — | A | — | — | — | M | — | — | 0.0665 | 2.3831 | 0.1268 | 3192 |
| 2876 | | | | | | | | | | | | | | | | | | |
| Phage- | T | L | S | M | — | V | — | T | — | — | — | T | — | — | 0.0958 | 2.3812 | 0.1145 | 3193 |
| 2877 | | | | | | | | | | | | | | | | | | |
| Phage- | E | S | A | R | — | V | — | M | — | — | — | N | — | — | 0.0515 | 2.3803 | 0.0714 | 3194 |
| 2878 | | | | | | | | | | | | | | | | | | |
| Phage- | V | T | — | I | — | K | — | Q | — | — | — | V | — | — | 0.0596 | 2.3795 | 0.0878 | 3195 |
| 2879 | | | | | | | | | | | | | | | | | | |
| Phage- | M | H | L | M | — | K | — | A | — | — | — | — | — | — | 0.0879 | 2.3773 | 0.1182 | 3196 |
| 2880 | | | | | | | | | | | | | | | | | | |
| Phage- | G | D | P | V | — | K | — | T | — | — | — | L | — | — | 0.0774 | 2.3768 | 0.09 | 3197 |
| 2881 | | | | | | | | | | | | | | | | | | |
| Phage- | P | E | R | M | — | I | — | S | — | — | — | E | V | L | 0.0622 | 2.3751 | 0.1001 | 3198 |
| 2882 | | | | | | | | | | | | | | | | | | |
| Phage- | W | D | A | K | — | V | — | R | — | — | — | D | — | — | 0.0611 | 2.3731 | 0.1253 | 3199 |
| 2883 | | | | | | | | | | | | | | | | | | |
| Phage- | E | F | M | R | — | T | — | P | — | — | — | L | — | — | 0.0718 | 2.3706 | 0.1196 | 3200 |
| 2884 | | | | | | | | | | | | | | | | | | |
| Phage- | N | P | P | Y | — | V | — | A | — | — | — | — | — | — | 0.0645 | 2.3695 | 0.0765 | 3201 |
| 2885 | | | | | | | | | | | | | | | | | | |
| Phage- | D | G | I | K | — | V | — | R | — | — | — | D | — | — | 0.0793 | 2.3619 | 0.1463 | 3202 |
| 2886 | | | | | | | | | | | | | | | | | | |
| Phage- | S | L | P | — | — | K | — | L | — | — | — | E | — | — | 0.0537 | 2.3604 | 0.0872 | 3203 |
| 2887 | | | | | | | | | | | | | | | | | | |
| Phage- | V | S | K | V | — | K | — | A | — | — | — | E | — | L | 0.0618 | 2.3593 | 0.104 | 3204 |
| 2888 | | | | | | | | | | | | | | | | | | |
| Phage- | M | — | S | — | — | V | — | S | — | — | — | T | — | — | 0.0603 | 2.3574 | 0.0629 | 3205 |
| 2889 | | | | | | | | | | | | | | | | | | |
| Phage- | Q | E | — | Y | — | K | — | A | — | — | — | D | — | — | 0.0804 | 2.3567 | 0.1038 | 3206 |
| 2890 | | | | | | | | | | | | | | | | | | |
| Phage- | E | — | H | I | — | R | — | L | — | — | — | T | — | — | 0.0716 | 2.3567 | 0.0899 | 3207 |
| 2891 | | | | | | | | | | | | | | | | | | |
| Phage- | E | T | P | F | — | I | — | Q | — | — | — | D | — | — | 0.0551 | 2.3547 | 0.1227 | 3208 |
| 2892 | | | | | | | | | | | | | | | | | | |
| Phage- | A | L | P | Q | — | V | — | A | — | — | — | L | — | — | 0.0578 | 2.349 | 0.0796 | 3209 |
| 2893 | | | | | | | | | | | | | | | | | | |
| Phage- | V | L | K | M | — | E | — | L | — | — | — | V | — | — | 0.061 | 2.3482 | 0.1175 | 3210 |
| 2894 | | | | | | | | | | | | | | | | | | |
| Phage- | Q | — | L | M | — | V | — | E | — | — | — | L | — | — | 0.0873 | 2.3477 | 0.1003 | 3211 |
| 2895 | | | | | | | | | | | | | | | | | | |
| Phage- | — | E | R | M | — | R | — | A | — | — | — | D | — | — | 0.061 | 2.3456 | 0.1881 | 3212 |
| 2896 | | | | | | | | | | | | | | | | | | |
| Phage- | H | D | K | — | — | R | — | A | — | — | — | N | — | — | 0.0771 | 2.3451 | 0.1068 | 3213 |
| 2897 | | | | | | | | | | | | | | | | | | |
| Phage- | N | W | P | V | — | K | — | M | — | — | — | T | — | — | 0.0815 | 2.3442 | 0.155 | 3214 |
| 2898 | | | | | | | | | | | | | | | | | | |
| Phage- | M | E | I | R | — | T | — | P | — | — | — | M | — | — | 0.0649 | 2.3422 | 0.0795 | 3215 |
| 2899 | | | | | | | | | | | | | | | | | | |
| Phage- | P | W | L | M | — | K | — | A | — | — | — | T | — | — | 0.111 | 2.3399 | 0.1829 | 3216 |
| 2900 | | | | | | | | | | | | | | | | | | |
| Phage- | V | T | P | K | — | V | — | M | — | — | — | — | — | — | 0.0554 | 2.3389 | 0.1234 | 3217 |
| 2901 | | | | | | | | | | | | | | | | | | |
| Phage- | Q | T | — | M | — | R | — | A | — | — | — | V | — | — | 0.0721 | 2.3383 | 0.092 | 3218 |
| 2902 | | | | | | | | | | | | | | | | | | |
| Phage- | V | T | P | A | — | V | — | T | — | — | — | Y | — | — | 0.0644 | 2.3367 | 0.1163 | 3219 |
| 2903 | | | | | | | | | | | | | | | | | | |
| Phage- | V | F | P | A | — | V | — | — | — | — | — | V | — | — | 0.0651 | 2.3325 | 0.1286 | 3220 |
| 2904 | | | | | | | | | | | | | | | | | | |
| Phage- | P | S | H | Y | — | V | — | A | — | — | — | — | — | — | 0.0717 | 2.3319 | 0.0875 | 3221 |
| 2905 | | | | | | | | | | | | | | | | | | |
| Phage- | V | E | P | S | — | V | — | Y | — | — | — | L | — | — | 0.0618 | 2.331 | 0.0835 | 3222 |
| 2906 | | | | | | | | | | | | | | | | | | |
| Phage- | A | E | M | R | — | I | — | E | — | — | — | S | — | — | 0.0712 | 2.3305 | 0.1185 | 3223 |
| 2907 | | | | | | | | | | | | | | | | | | |
| Phage- | L | G | L | A | — | I | — | E | — | — | — | V | — | — | 0.0689 | 2.3288 | 0.0789 | 3224 |
| 2908 | | | | | | | | | | | | | | | | | | |
| Phage- | G | E | P | V | — | K | — | A | — | — | — | E | — | — | 0.0594 | 2.3284 | 0.1281 | 3225 |
| 2909 | | | | | | | | | | | | | | | | | | |
| Phage- | A | V | P | — | — | M | — | K | — | — | — | T | — | — | 0.0656 | 2.3282 | 0.0971 | 3226 |
| 2910 | | | | | | | | | | | | | | | | | | |
| Phage- | E | K | P | V | — | V | — | S | — | — | — | T | — | — | 0.0624 | 2.3272 | 0.098 | 3227 |
| 2911 | | | | | | | | | | | | | | | | | | |
| Phage- | A | V | H | Y | — | — | — | S | — | — | — | T | — | — | 0.0757 | 2.3254 | 0.0814 | 3228 |
| 2912 | | | | | | | | | | | | | | | | | | |
| Phage- | G | E | R | W | — | T | — | P | — | — | — | V | — | L | 0.0716 | 2.3234 | 0.1164 | 3229 |
| 2913 | | | | | | | | | | | | | | | | | | |
| Phage- | G | L | A | — | — | K | — | E | — | — | — | I | — | — | 0.0611 | 2.3218 | 0.0815 | 3230 |
| 2914 | | | | | | | | | | | | | | | | | | |
| Phage- | G | G | L | — | — | K | — | A | — | — | — | N | W | L | 0.118 | 2.3211 | 0.1155 | 3231 |
| 2915 | | | | | | | | | | | | | | | | | | |
| Phage- | V | D | R | M | — | V | — | K | — | — | — | E | — | L | 0.0733 | 2.3206 | 0.0971 | 3232 |
| 2916 | | | | | | | | | | | | | | | | | | |
| Phage- | L | D | — | — | — | K | — | T | — | — | — | M | — | — | 0.0596 | 2.3178 | 0.0821 | 3233 |
| 2917 | | | | | | | | | | | | | | | | | | |
| Phage- | L | P | R | I | — | V | — | E | — | — | — | L | — | — | 0.0597 | 2.3165 | 0.0919 | 3234 |
| 2918 | | | | | | | | | | | | | | | | | | |
| Phage- | V | D | P | M | — | I | — | R | — | — | — | S | F | L | 0.066 | 2.3157 | 0.0867 | 3235 |
| 2919 | | | | | | | | | | | | | | | | | | |
| Phage- | L | — | I | M | — | R | — | A | — | — | — | S | — | — | 0.0739 | 2.315 | 0.1117 | 3236 |
| 2920 | | | | | | | | | | | | | | | | | | |
| Phage- | — | S | H | M | — | K | — | V | — | — | — | S | — | L | 0.0659 | 2.3134 | 0.125 | 3237 |
| 2921 | | | | | | | | | | | | | | | | | | |
| Phage- | V | W | P | R | — | E | — | Q | — | — | — | — | — | — | 0.0724 | 2.3132 | 0.0854 | 3238 |
| 2923 | | | | | | | | | | | | | | | | | | |
| Phage- | V | P | K | M | — | T | — | P | — | — | — | W | — | — | 0.0586 | 2.3091 | 0.0934 | 3239 |
| 2923 | | | | | | | | | | | | | | | | | | |
| Phage- | V | G | K | M | — | V | — | E | — | — | — | H | — | — | 0.0626 | 2.3086 | 0.082 | 3240 |
| 2924 | | | | | | | | | | | | | | | | | | |
| Phage- | T | P | I | F | — | V | — | A | — | — | — | S | — | — | 0.1203 | 2.3071 | 0.0946 | 3241 |
| 2925 | | | | | | | | | | | | | | | | | | |
| Phage- | V | D | R | M | — | K | — | T | — | — | — | E | — | — | 0.0642 | 2.3066 | 0.1197 | 3242 |
| 2926 | | | | | | | | | | | | | | | | | | |
| Phage- | — | K | P | F | — | V | — | E | — | — | — | V | A | L | 0.0592 | 2.3065 | 0.1213 | 3243 |
| 2927 | | | | | | | | | | | | | | | | | | |
| Phage- | — | V | P | A | — | K | — | V | — | — | — | D | — | — | 0.0679 | 2.306 | 0.1102 | 3244 |
| 2928 | | | | | | | | | | | | | | | | | | |
| Phage- | N | I | P | H | — | V | — | A | — | — | — | I | — | — | 0.0593 | 2.3055 | 0.1093 | 3245 |
| 2929 | | | | | | | | | | | | | | | | | | |
| Phage- | T | Q | A | — | — | V | — | S | — | — | — | D | — | — | 0.0507 | 2.3032 | 0.0747 | 3246 |
| 2930 | | | | | | | | | | | | | | | | | | |
| Phage- | V | E | Q | M | — | K | — | E | — | — | — | K | — | — | 0.0661 | 2.3016 | 0.1088 | 3247 |
| 2931 | | | | | | | | | | | | | | | | | | |
| Phage- | M | H | M | — | — | R | — | A | — | — | — | S | — | — | 0.1185 | 2.2948 | 0.2099 | 3248 |
| 2932 | | | | | | | | | | | | | | | | | | |
| Phage- | V | E | R | A | — | V | — | V | — | — | — | E | — | — | 0.0624 | 2.2931 | 0.11 | 3249 |
| 2933 | | | | | | | | | | | | | | | | | | |
| Phage- | E | F | N | W | — | R | — | L | — | — | — | V | — | — | 0.1555 | 2.2919 | 0.1566 | 3250 |
| 2934 | | | | | | | | | | | | | | | | | | |
| Phage- | — | H | P | V | — | K | — | D | — | — | — | — | — | — | 0.0748 | 2.2914 | 0.0823 | 3251 |
| 2935 | | | | | | | | | | | | | | | | | | |
| Phage- | — | D | P | M | — | K | — | L | — | — | — | — | — | — | 0.0685 | 2.2906 | 0.1449 | 3252 |
| 2936 | | | | | | | | | | | | | | | | | | |
| Phage- | V | F | P | M | — | K | — | A | — | — | — | L | — | — | 0.1459 | 2.2904 | 0.3194 | 3253 |
| 2937 | | | | | | | | | | | | | | | | | | |
| Phage- | H | V | P | K | — | V | — | Q | — | — | — | I | — | — | 0.1165 | 2.2898 | 0.2015 | 3254 |
| 2938 | | | | | | | | | | | | | | | | | | |
| Phage- | — | S | A | — | — | V | — | D | — | — | — | Y | — | — | 0.0658 | 2.2841 | 0.6963 | 3255 |
| 2939 | | | | | | | | | | | | | | | | | | |
| Phage- | F | D | K | I | — | V | — | T | — | — | — | T | — | — | 0.062 | 2.2812 | 0.1068 | 3256 |
| 2940 | | | | | | | | | | | | | | | | | | |
| Phage- | D | V | R | M | — | K | — | S | — | — | — | I | — | L | 0.0669 | 2.2804 | 0.0754 | 3257 |
| 2941 | | | | | | | | | | | | | | | | | | |
| Phage- | E | L | I | — | — | — | — | R | — | — | — | E | — | — | 0.0506 | 2.2798 | 0.0637 | 3258 |
| 2942 | | | | | | | | | | | | | | | | | | |
| Phage- | E | G | I | Y | — | R | — | A | — | — | — | E | — | — | 0.0512 | 2.2798 | 0.0765 | 3259 |
| 2943 | | | | | | | | | | | | | | | | | | |
| Phage- | L | T | P | M | — | K | — | S | — | — | — | D | — | — | 0.0652 | 2.279 | 0.1049 | 3260 |
| 2944 | | | | | | | | | | | | | | | | | | |
| Phage- | T | V | I | K | — | I | — | A | — | — | — | D | — | — | 0.0589 | 2.2786 | 0.09 | 3261 |
| 2945 | | | | | | | | | | | | | | | | | | |
| Phage- | E | M | R | M | — | T | — | P | — | — | — | W | — | — | 0.0546 | 2.2785 | 0.1123 | 3262 |
| 2946 | | | | | | | | | | | | | | | | | | |
| Phage- | G | D | H | — | — | R | — | S | — | — | — | V | — | — | 0.0555 | 2.2781 | 0.0649 | 3263 |
| 2947 | | | | | | | | | | | | | | | | | | |
| Phage- | F | P | H | M | — | K | — | E | — | — | — | — | — | — | 0.065 | 2.2771 | 0.0876 | 3264 |
| 2948 | | | | | | | | | | | | | | | | | | |
| Phage- | W | D | H | — | — | K | — | D | — | — | — | L | — | — | 0.0564 | 2.2768 | 0.0766 | 3265 |
| 2949 | | | | | | | | | | | | | | | | | | |
| Phage- | S | S | P | I | — | K | — | M | — | — | — | T | — | — | 0.0584 | 2.2752 | 0.0918 | 3266 |
| 2950 | | | | | | | | | | | | | | | | | | |
| Phage- | E | N | I | M | — | E | — | M | — | — | — | I | — | — | 0.0664 | 2.2737 | 0.0839 | 3267 |
| 2951 | | | | | | | | | | | | | | | | | | |
| Phage- | — | M | P | K | — | E | — | I | — | — | — | — | — | — | 0.0598 | 2.2733 | 0.0746 | 3268 |
| 2952 | | | | | | | | | | | | | | | | | | |
| Phage- | V | D | I | Q | — | K | — | L | — | — | — | I | — | M | 0.0554 | 2.2712 | 0.0643 | 3269 |
| 2953 | | | | | | | | | | | | | | | | | | |
| Phage- | T | L | M | R | — | E | — | S | — | — | — | Q | — | — | 0.0807 | 2.2711 | 0.0698 | 3270 |
| 2954 | | | | | | | | | | | | | | | | | | |
| Phage- | T | T | A | Y | — | I | — | P | — | — | — | S | — | — | 0.0816 | 2.2708 | 0.15 | 3271 |
| 2955 | | | | | | | | | | | | | | | | | | |
| Phage- | Y | L | Q | F | — | K | — | V | — | — | — | T | — | — | 0.1279 | 2.2668 | 0.1498 | 3272 |
| 2956 | | | | | | | | | | | | | | | | | | |
| Phage- | A | Y | E | M | — | I | — | A | — | — | — | E | — | — | 0.0529 | 2.2654 | 0.0905 | 3273 |
| 2957 | | | | | | | | | | | | | | | | | | |
| Phage- | E | Q | P | — | — | T | — | Q | — | — | — | W | — | — | 0.0686 | 2.265 | 0.0972 | 3274 |
| 2958 | | | | | | | | | | | | | | | | | | |
| Phage- | P | E | F | V | — | K | — | S | — | — | — | Q | — | — | 0.054 | 2.2649 | 0.0681 | 3275 |
| 2959 | | | | | | | | | | | | | | | | | | |
| Phage- | W | E | Q | R | — | V | — | E | — | — | — | V | — | — | 0.0522 | 2.2635 | 0.0987 | 3276 |
| 2960 | | | | | | | | | | | | | | | | | | |
| Phage- | A | D | P | V | — | R | — | Q | — | — | — | E | — | — | 0.0844 | 2.2621 | 0.1097 | 3277 |
| 2961 | | | | | | | | | | | | | | | | | | |
| Phage- | V | Q | S | — | — | V | — | Y | — | — | — | T | — | — | 0.0577 | 2.2611 | 0.1024 | 3278 |
| 2962 | | | | | | | | | | | | | | | | | | |
| Phage- | N | L | P | — | — | K | — | A | — | — | — | K | W | L | 0.1205 | 2.2608 | 0.1253 | 3279 |
| 2963 | | | | | | | | | | | | | | | | | | |
| Phage- | P | G | P | — | — | V | — | E | — | — | — | I | — | — | 0.0592 | 2.2604 | 0.1138 | 3280 |
| 2964 | | | | | | | | | | | | | | | | | | |
| Phage- | L | F | K | M | — | — | — | S | — | — | — | D | — | — | 0.0517 | 2.2585 | 0.0804 | 3281 |
| 2965 | | | | | | | | | | | | | | | | | | |
| Phage- | Q | T | P | V | — | I | — | E | — | — | — | T | — | — | 0.0736 | 2.2584 | 0.091 | 3282 |
| 2966 | | | | | | | | | | | | | | | | | | |
| Phage- | G | E | R | Y | — | V | — | A | — | — | — | — | — | L | 0.0554 | 2.2577 | 0.1062 | 3283 |
| 2967 | | | | | | | | | | | | | | | | | | |
| Phage- | W | E | A | M | — | — | — | L | — | — | — | T | — | — | 0.0715 | 2.2565 | 0.0968 | 3284 |
| 2968 | | | | | | | | | | | | | | | | | | |
| Phage- | L | D | I | W | — | V | — | A | — | — | — | H | — | — | 0.0523 | 2.2563 | 0.0712 | 3285 |
| 2969 | | | | | | | | | | | | | | | | | | |
| Phage- | V | Y | P | — | — | K | — | S | — | — | — | S | — | L | 0.0644 | 2.2558 | 0.0843 | 3286 |
| 2970 | | | | | | | | | | | | | | | | | | |
| Phage- | D | I | P | I | — | R | — | Q | — | — | — | I | — | — | 0.0781 | 2.2521 | 0.0617 | 3287 |
| 2971 | | | | | | | | | | | | | | | | | | |
| Phage- | G | D | — | Y | — | K | — | A | — | — | — | D | — | — | 0.0527 | 2.2514 | 0.0762 | 3288 |
| 2972 | | | | | | | | | | | | | | | | | | |
| Phage- | V | S | P | — | — | V | — | S | — | — | — | V | — | — | 0.0627 | 2.2497 | 0.108 | 3289 |
| 2973 | | | | | | | | | | | | | | | | | | |
| Phage- | V | G | A | A | — | I | — | D | — | — | — | V | — | — | 0.078 | 2.2462 | 0.0732 | 3290 |
| 2974 | | | | | | | | | | | | | | | | | | |
| Phage- | G | V | P | M | — | V | — | D | — | — | — | H | — | L | 0.0584 | 2.244 | 0.073 | 3291 |
| 2975 | | | | | | | | | | | | | | | | | | |
| Phage- | F | E | M | — | — | K | — | A | — | — | — | S | — | — | 0.0649 | 2.2434 | 0.0857 | 3292 |
| 2976 | | | | | | | | | | | | | | | | | | |
| Phage- | T | E | L | R | — | V | — | S | — | — | — | T | — | — | 0.0775 | 2.2398 | 0.0676 | 3293 |
| 2977 | | | | | | | | | | | | | | | | | | |
| Phage- | F | G | Y | A | — | I | — | — | — | — | — | T | V | — | 0.0813 | 2.239 | 0.1222 | 3294 |
| 2978 | | | | | | | | | | | | | | | | | | |
| Phage- | S | E | I | A | — | R | — | A | — | — | — | Q | W | L | 0.0808 | 2.2385 | 0.1251 | 3295 |
| 2979 | | | | | | | | | | | | | | | | | | |
| Phage- | V | E | H | W | — | R | — | E | — | — | — | — | — | — | 0.0628 | 2.238 | 0.0893 | 3296 |
| 2980 | | | | | | | | | | | | | | | | | | |
| Phage- | D | D | R | M | — | I | — | S | — | — | — | S | — | L | 0.063 | 2.2353 | 0.0851 | 3297 |
| 2981 | | | | | | | | | | | | | | | | | | |
| Phage- | P | M | H | A | — | I | — | M | — | — | — | — | — | — | 0.0634 | 2.2342 | 0.1144 | 3298 |
| 2982 | | | | | | | | | | | | | | | | | | |
| Phage- | P | E | R | M | — | R | — | L | — | — | — | E | — | — | 0.0494 | 2.2306 | 0.1099 | 3299 |
| 2983 | | | | | | | | | | | | | | | | | | |
| Phage- | A | G | I | R | — | V | — | E | — | — | — | L | — | — | 0.0682 | 2.2282 | 0.0784 | 3300 |
| 2984 | | | | | | | | | | | | | | | | | | |
| Phage- | E | E | — | Y | — | V | — | K | — | — | — | L | — | — | 0.0602 | 2.2275 | 0.0871 | 3301 |
| 2985 | | | | | | | | | | | | | | | | | | |
| Phage- | S | Q | P | M | — | I | — | V | — | — | — | R | — | — | 0.0622 | 2.2274 | 0.1093 | 3302 |
| 2986 | | | | | | | | | | | | | | | | | | |
| Phage- | L | L | — | R | — | — | — | E | — | — | — | V | — | — | 0.0564 | 2.2261 | 0.1404 | 3303 |
| 2987 | | | | | | | | | | | | | | | | | | |
| Phage- | F | T | A | Y | — | K | — | A | — | — | — | Q | — | L | 0.0991 | 2.2243 | 0.0917 | 3304 |
| 2988 | | | | | | | | | | | | | | | | | | |
| Phage- | T | V | — | M | — | K | — | S | — | — | — | E | — | — | 0.0643 | 2.224 | 0.083 | 3305 |
| 2989 | | | | | | | | | | | | | | | | | | |
| Phage- | T | T | I | M | — | E | — | T | — | — | — | T | — | — | 0.0655 | 2.2233 | 0.0683 | 3306 |
| 2990 | | | | | | | | | | | | | | | | | | |
| Phage- | F | H | — | R | — | V | — | E | — | — | — | W | — | — | 0.1073 | 2.2232 | 0.1532 | 3307 |
| 2991 | | | | | | | | | | | | | | | | | | |
| Phage- | Y | E | I | R | — | E | — | K | — | — | — | M | — | — | 0.0536 | 2.2228 | 0.0735 | 3308 |
| 2992 | | | | | | | | | | | | | | | | | | |
| Phage- | L | — | H | M | — | I | — | A | — | — | — | — | — | L | 0.0782 | 2.2227 | 0.1037 | 3309 |
| 2993 | | | | | | | | | | | | | | | | | | |
| Phage- | L | Q | S | M | — | R | — | A | — | — | — | T | — | — | 0.0703 | 2.2226 | 0.0839 | 3310 |
| 2994 | | | | | | | | | | | | | | | | | | |
| Phage- | A | I | A | H | — | I | — | Q | — | — | — | S | — | — | 0.0606 | 2.2219 | 0.0878 | 3311 |
| 2995 | | | | | | | | | | | | | | | | | | |
| Phage- | P | E | A | — | — | M | — | P | — | — | — | H | — | — | 0.051 | 2.2212 | 0.0657 | 3312 |
| 2996 | | | | | | | | | | | | | | | | | | |
| Phage- | M | S | P | R | — | V | — | M | — | — | — | D | W | L | 0.0628 | 2.2207 | 0.0821 | 3313 |
| 2997 | | | | | | | | | | | | | | | | | | |
| Phage- | V | V | A | H | — | V | — | S | — | — | — | T | — | — | 0.0755 | 2.2206 | 0.0901 | 3314 |
| 2998 | | | | | | | | | | | | | | | | | | |
| Phage- | — | — | R | M | — | V | — | E | — | — | — | Q | E | L | 0.0606 | 2.2181 | 0.1074 | 3315 |
| 2999 | | | | | | | | | | | | | | | | | | |
| Phage- | E | I | P | V | — | R | — | A | — | — | — | L | — | L | 0.0584 | 2.2173 | 0.0698 | 3316 |
| 3000 | | | | | | | | | | | | | | | | | | |
| Phage | M | M | P | — | — | M | — | T | — | — | — | E | — | — | 0.0545 | 2.217 | 0.075 | 3317 |
| 3001 | | | | | | | | | | | | | | | | | | |
| Phage- | Y | Q | L | — | — | K | — | E | — | — | — | V | — | — | 0.0535 | 2.2143 | 0.0672 | 3318 |
| 3002 | | | | | | | | | | | | | | | | | | |
| Phage- | M | L | P | W | — | V | — | H | — | — | — | D | — | — | 0.0916 | 2.2141 | 0.1321 | 3319 |
| 3003 | | | | | | | | | | | | | | | | | | |
| Phage- | V | E | P | M | — | R | — | A | — | — | — | L | — | — | 0.0854 | 2.2135 | 0.0882 | 3320 |
| 3004 | | | | | | | | | | | | | | | | | | |
| Phage- | A | L | P | M | — | E | — | F | — | — | — | T | — | — | 0.0647 | 2.2132 | 0.0628 | 3321 |
| 3005 | | | | | | | | | | | | | | | | | | |
| Phage- | V | M | — | E | — | R | — | A | — | — | — | N | — | — | 0.0712 | 2.213 | 0.0636 | 3322 |
| 3006 | | | | | | | | | | | | | | | | | | |
| Phage- | M | Y | H | M | — | R | — | S | — | — | — | E | — | — | 0.0675 | 2.2123 | 0.142 | 3323 |
| 3007 | | | | | | | | | | | | | | | | | | |
| Phage- | S | E | — | M | — | V | — | Q | — | — | — | N | — | — | 0.0664 | 2.2108 | 0.0981 | 3324 |
| 3008 | | | | | | | | | | | | | | | | | | |
| Phage- | W | W | P | S | — | V | — | E | — | — | — | — | — | L | 0.167 | 2.2102 | 0.1287 | 3325 |
| 3009 | | | | | | | | | | | | | | | | | | |
| Phage- | G | M | P | Y | — | V | — | E | — | — | — | S | W | L | 0.0538 | 2.21 | 0.0827 | 3326 |
| 3010 | | | | | | | | | | | | | | | | | | |
| Phage- | W | E | P | V | — | K | — | D | — | — | — | N | — | — | 0.0671 | 2.2094 | 0.08 | 3327 |
| 3011 | | | | | | | | | | | | | | | | | | |
| Phage- | Q | W | L | R | — | E | — | E | — | — | — | I | — | — | 0.0683 | 2.2084 | 0.2123 | 3328 |
| 3012 | | | | | | | | | | | | | | | | | | |
| Phage- | S | T | M | M | — | V | — | P | — | — | — | M | — | — | 0.053 | 2.2075 | 0.0775 | 3329 |
| 3013 | | | | | | | | | | | | | | | | | | |
| Phage- | A | E | L | — | — | V | — | T | — | — | — | M | — | — | 0.0659 | 2.2037 | 0.0679 | 3330 |
| 3014 | | | | | | | | | | | | | | | | | | |
| Phage- | P | — | E | W | — | I | — | V | — | — | — | T | — | — | 0.066 | 2.2035 | 0.0732 | 3331 |
| 3015 | | | | | | | | | | | | | | | | | | |
| Phage- | A | N | — | A | — | I | — | P | — | — | — | L | — | — | 0.0685 | 2.2032 | 0.1092 | 3332 |
| 3016 | | | | | | | | | | | | | | | | | | |
| Phage- | V | Q | G | V | — | V | — | S | — | — | — | T | — | — | 0.1008 | 2.199 | 0.0875 | 3333 |
| 3017 | | | | | | | | | | | | | | | | | | |
| Phage- | H | E | A | M | — | R | — | T | — | — | — | H | — | — | 0.0689 | 2.1986 | 0.0722 | 3334 |
| 3018 | | | | | | | | | | | | | | | | | | |
| Phage- | — | E | S | R | — | V | — | E | — | — | — | Y | — | — | 0.0572 | 2.1984 | 0.0726 | 3335 |
| 3019 | | | | | | | | | | | | | | | | | | |
| Phage- | E | E | K | M | — | T | — | Q | — | — | — | S | — | — | 0.0651 | 2.1982 | 0.0688 | 3336 |
| 3020 | | | | | | | | | | | | | | | | | | |
| Phage- | G | G | L | R | — | E | — | M | — | — | — | H | — | — | 0.0721 | 2.1967 | 0.1221 | 3337 |
| 3021 | | | | | | | | | | | | | | | | | | |
| Phage- | D | H | L | M | — | K | — | S | — | — | — | K | — | — | 0.0636 | 2.1947 | 0.0924 | 3338 |
| 3022 | | | | | | | | | | | | | | | | | | |
| Phage- | T | V | P | K | — | V | — | — | — | — | — | D | W | L | 0.068 | 2.1933 | 0.0864 | 3339 |
| 3023 | | | | | | | | | | | | | | | | | | |
| Phage- | — | E | R | — | — | M | — | M | — | — | — | T | — | L | 0.053 | 2.1911 | 0.0679 | 3340 |
| 3024 | | | | | | | | | | | | | | | | | | |
| Phage- | P | L | — | V | — | K | — | A | — | — | — | I | — | — | 0.0679 | 2.191 | 0.0979 | 3341 |
| 3025 | | | | | | | | | | | | | | | | | | |
| Phage- | V | D | — | — | — | V | — | S | — | — | — | S | W | L | 0.059 | 2.1892 | 0.0683 | 3342 |
| 3026 | | | | | | | | | | | | | | | | | | |
| Phage- | V | L | P | V | — | K | — | L | — | — | — | K | — | L | 0.147 | 2.1886 | 0.2341 | 3343 |
| 3027 | | | | | | | | | | | | | | | | | | |
| Phage- | D | M | R | R | — | E | — | I | — | — | — | V | — | — | 0.0718 | 2.1883 | 0.0954 | 3344 |
| 3028 | | | | | | | | | | | | | | | | | | |
| Phage- | D | S | I | M | — | R | — | A | — | — | — | — | — | — | 0.0612 | 2.1875 | 0.0945 | 3345 |
| 3029 | | | | | | | | | | | | | | | | | | |
| Phage- | H | H | Q | — | — | K | — | L | — | — | — | E | — | — | 0.0795 | 2.1861 | 0.13 | 3346 |
| 3030 | | | | | | | | | | | | | | | | | | |
| Phage- | W | E | I | — | — | K | — | E | — | — | — | S | — | — | 0.0638 | 2.1857 | 0.1051 | 3347 |
| 3031 | | | | | | | | | | | | | | | | | | |
| Phage- | E | G | — | V | — | I | — | E | — | — | — | K | — | — | 0.0555 | 2.1846 | 0.0637 | 3348 |
| 3032 | | | | | | | | | | | | | | | | | | |
| Phage- | E | L | L | M | — | R | — | A | — | — | — | D | — | — | 0.0798 | 2.1821 | 0.087 | 3349 |
| 3033 | | | | | | | | | | | | | | | | | | |
| Phage- | Y | — | H | I | — | K | — | S | — | — | — | D | — | — | 0.0719 | 2.1812 | 0.108 | 3350 |
| 3034 | | | | | | | | | | | | | | | | | | |
| Phage- | L | M | P | — | — | V | — | V | — | — | — | S | — | — | 0.0657 | 2.1792 | 0.1063 | 3351 |
| 3035 | | | | | | | | | | | | | | | | | | |
| Phage- | — | I | P | A | — | V | — | P | — | — | — | S | — | — | 0.0769 | 2.179 | 0.2123 | 3352 |
| 3036 | | | | | | | | | | | | | | | | | | |
| Phage- | P | — | — | R | — | E | — | S | — | — | — | Q | — | — | 0.073 | 2.1781 | 0.1606 | 3353 |
| 3037 | | | | | | | | | | | | | | | | | | |
| Phage- | V | E | P | Q | — | R | — | A | — | — | — | Y | — | — | 0.0565 | 2.177 | 0.0668 | 3354 |
| 3038 | | | | | | | | | | | | | | | | | | |
| Phage- | V | H | N | W | — | V | — | S | — | — | — | L | — | L | 0.0952 | 2.1759 | 0.099 | 3355 |
| 3039 | | | | | | | | | | | | | | | | | | |
| Phage- | D | Y | P | — | — | R | — | E | — | — | — | M | — | — | 0.062 | 2.1751 | 0.0813 | 3356 |
| 3040 | | | | | | | | | | | | | | | | | | |
| Phage- | E | H | L | R | — | V | — | D | — | — | — | V | — | — | 0.0569 | 2.1749 | 0.0886 | 3357 |
| 3041 | | | | | | | | | | | | | | | | | | |
| Phage- | Y | T | M | V | — | R | — | A | — | — | — | Q | — | — | 0.0666 | 2.172 | 0.0964 | 3358 |
| 3042 | | | | | | | | | | | | | | | | | | |
| Phage- | P | T | — | Y | — | K | — | E | — | — | — | T | — | — | 0.0572 | 2.1718 | 0.0869 | 3359 |
| 3043 | | | | | | | | | | | | | | | | | | |
| Phage- | S | L | A | V | — | K | — | S | — | — | — | — | — | — | 0.0615 | 2.1714 | 0.0813 | 3360 |
| 3044 | | | | | | | | | | | | | | | | | | |
| Phage- | V | I | G | W | — | V | — | Q | — | — | — | S | — | — | 0.0809 | 2.1713 | 0.1131 | 3361 |
| 3045 | | | | | | | | | | | | | | | | | | |
| Phage- | V | D | K | V | — | R | — | M | — | — | — | — | — | — | 0.1215 | 2.1697 | 0.1262 | 3362 |
| 3046 | | | | | | | | | | | | | | | | | | |
| Phage- | V | G | N | M | — | V | — | E | — | — | — | V | — | — | 0.053 | 2.1687 | 0.0878 | 3363 |
| 3047 | | | | | | | | | | | | | | | | | | |
| Phage- | P | G | H | V | — | R | — | A | — | — | — | E | — | — | 0.0695 | 2.1674 | 0.106 | 3364 |
| 3048 | | | | | | | | | | | | | | | | | | |
| Phage- | V | N | — | K | — | T | — | P | — | — | — | F | — | — | 0.0894 | 2.1619 | 0.0865 | 3365 |
| 3049 | | | | | | | | | | | | | | | | | | |
| Phage- | E | L | Y | K | — | V | — | S | — | — | — | Y | — | — | 0.0626 | 2.1619 | 0.0696 | 3366 |
| 3050 | | | | | | | | | | | | | | | | | | |
| Phage- | P | G | Q | V | — | I | — | E | — | — | — | T | — | — | 0.0638 | 2.1616 | 0.0874 | 3367 |
| 3051 | | | | | | | | | | | | | | | | | | |
| Phage- | D | M | R | — | — | R | — | V | — | — | — | E | — | — | 0.0785 | 2.1606 | 0.1295 | 3368 |
| 3052 | | | | | | | | | | | | | | | | | | |
| Phage- | V | — | P | S | — | V | — | L | — | — | — | V | — | M | 0.0634 | 2.16 | 0.6797 | 3369 |
| 3053 | | | | | | | | | | | | | | | | | | |
| Phage- | D | W | I | R | — | L | — | A | — | — | — | T | — | — | 0.0768 | 2.159 | 0.0848 | 3370 |
| 3054 | | | | | | | | | | | | | | | | | | |
| Phage- | P | V | E | — | — | R | — | A | — | — | — | W | — | — | 0.0617 | 2.1578 | 0.1027 | 3371 |
| 3055 | | | | | | | | | | | | | | | | | | |
| Phage- | — | E | M | M | — | R | — | S | — | — | — | D | W | L | 0.0569 | 2.1575 | 0.0844 | 3372 |
| 3056 | | | | | | | | | | | | | | | | | | |
| Phage- | T | G | P | V | — | I | — | K | — | — | — | F | — | — | 0.0543 | 2.1534 | 0.075 | 3373 |
| 3057 | | | | | | | | | | | | | | | | | | |
| Phage- | D | W | — | R | — | V | — | E | — | — | — | L | — | — | 0.0618 | 2.1507 | 0.1059 | 3374 |
| 3058 | | | | | | | | | | | | | | | | | | |
| Phage- | E | F | A | M | — | R | — | V | — | — | — | W | — | — | 0.148 | 2.1494 | 0.142 | 3375 |
| 3059 | | | | | | | | | | | | | | | | | | |
| Phage- | E | E | K | M | — | V | — | L | — | — | — | S | — | — | 0.0613 | 2.1491 | 0.0787 | 3376 |
| 3060 | | | | | | | | | | | | | | | | | | |
| Phage- | — | T | K | A | — | I | — | E | — | — | — | H | A | — | 0.1069 | 2.1471 | 0.0651 | 3377 |
| 3061 | | | | | | | | | | | | | | | | | | |
| Phage- | V | Q | — | — | — | V | — | S | — | — | — | H | — | — | 0.0557 | 2.1454 | 0.0688 | 3378 |
| 3062 | | | | | | | | | | | | | | | | | | |
| Phage- | V | E | I | Q | — | R | — | S | — | — | — | M | — | — | 0.064 | 2.1401 | 0.0951 | 3379 |
| 3063 | | | | | | | | | | | | | | | | | | |
| Phage- | V | V | P | M | — | S | — | S | — | — | — | Q | — | — | 0.0574 | 2.1389 | 0.0725 | 3380 |
| 3064 | | | | | | | | | | | | | | | | | | |
| Phage- | G | M | — | R | — | V | — | Q | — | — | — | V | — | — | 0.0803 | 2.1361 | 0.111 | 3381 |
| 3065 | | | | | | | | | | | | | | | | | | |
| Phage- | V | E | R | S | — | V | — | S | — | — | — | Y | — | — | 0.057 | 2.1352 | 0.0806 | 3382 |
| 3066 | | | | | | | | | | | | | | | | | | |
| Phage- | L | L | A | E | — | V | A | A | — | — | — | S | — | — | 0.0592 | 2.1329 | 0.0712 | 3383 |
| 3067 | | | | | | | | | | | | | | | | | | |
| Phage- | L | S | H | M | — | R | — | A | — | — | — | E | — | — | 0.0772 | 2.1321 | 0.0938 | 3384 |
| 3068 | | | | | | | | | | | | | | | | | | |
| Phage- | — | S | P | V | — | I | — | S | — | — | — | L | — | L | 0.0627 | 2.1306 | 0.1739 | 3385 |
| 3069 | | | | | | | | | | | | | | | | | | |
| Phage- | L | E | K | A | — | V | — | Q | — | — | — | Y | — | L | 0.0754 | 2.1292 | 0.0938 | 3386 |
| 3070 | | | | | | | | | | | | | | | | | | |
| Phage- | S | P | Y | F | — | I | — | A | — | — | — | H | — | — | 0.0735 | 2.1276 | 0.1213 | 3387 |
| 3071 | | | | | | | | | | | | | | | | | | |
| Phage- | — | M | M | R | — | E | — | V | — | — | — | E | — | — | 0.0623 | 2.1253 | 0.1176 | 3388 |
| 3072 | | | | | | | | | | | | | | | | | | |
| Phage- | W | L | M | R | — | E | — | Q | — | — | — | — | — | — | 0.1233 | 2.1244 | 0.131 | 3389 |
| 3073 | | | | | | | | | | | | | | | | | | |
| Phage- | T | — | S | W | — | K | — | G | G | A | — | K | — | — | 0.0886 | 2.1241 | 0.0801 | 3390 |
| 3074 | | | | | | | | | | | | | | | | | | |
| Phage- | Y | E | Q | — | — | K | — | L | — | — | — | Q | — | — | 0.0508 | 2.1219 | 0.0628 | 3391 |
| 3075 | | | | | | | | | | | | | | | | | | |
| Phage- | D | E | W | I | — | R | — | A | — | — | — | Q | — | — | 0.0578 | 2.1172 | 0.0632 | 3392 |
| 3076 | | | | | | | | | | | | | | | | | | |
| Phage- | V | N | H | — | — | K | — | T | — | — | — | — | — | — | 0.058 | 2.1169 | 0.0765 | 3393 |
| 3077 | | | | | | | | | | | | | | | | | | |
| Phage- | T | Y | M | M | — | V | — | E | — | — | — | Q | — | — | 0.0635 | 2.1137 | 0.1013 | 3394 |
| 3078 | | | | | | | | | | | | | | | | | | |
| Phage- | H | L | M | A | — | K | — | A | — | — | — | L | — | L | 0.0941 | 2.1133 | 0.1346 | 3395 |
| 3079 | | | | | | | | | | | | | | | | | | |
| Phage- | E | W | M | K | — | V | — | L | — | — | — | E | — | I | 0.0888 | 2.1127 | 0.0802 | 3396 |
| 3080 | | | | | | | | | | | | | | | | | | |
| Phage- | T | D | H | — | — | V | — | S | — | — | — | L | — | — | 0.0575 | 2.1106 | 0.0801 | 3397 |
| 3081 | | | | | | | | | | | | | | | | | | |
| Phage- | D | G | P | — | — | V | — | Q | — | — | — | L | W | L | 0.0547 | 2.1101 | 0.0699 | 3398 |
| 3082 | | | | | | | | | | | | | | | | | | |
| Phage- | G | Q | N | — | — | I | — | S | — | — | — | E | — | — | 0.058 | 2.11 | 0.08 | 3399 |
| 3083 | | | | | | | | | | | | | | | | | | |
| Phage- | L | D | A | M | — | R | — | L | — | — | — | S | — | — | 0.073 | 2.1053 | 0.0799 | 3400 |
| 3084 | | | | | | | | | | | | | | | | | | |
| Phage- | L | E | H | M | — | R | — | S | — | — | — | T | — | — | 0.0749 | 2.1036 | 0.0798 | 3401 |
| 3085 | | | | | | | | | | | | | | | | | | |
| Phage- | F | W | A | Q | — | V | — | P | — | — | — | S | — | — | 0.1355 | 2.1033 | 0.1994 | 3402 |
| 3086 | | | | | | | | | | | | | | | | | | |
| Phage- | V | D | L | I | — | R | — | A | — | — | — | S | — | — | 0.0552 | 2.1031 | 0.0726 | 3403 |
| 3087 | | | | | | | | | | | | | | | | | | |
| Phage- | V | V | P | R | — | E | — | M | — | — | — | Q | — | — | 0.0642 | 2.1017 | 0.0982 | 3404 |
| 3088 | | | | | | | | | | | | | | | | | | |
| Phage- | T | D | Q | — | — | K | — | L | — | — | — | T | — | — | 0.0665 | 2.1013 | 0.0815 | 3405 |
| 3089 | | | | | | | | | | | | | | | | | | |
| Phage- | V | S | I | M | — | T | — | L | — | — | — | E | — | — | 0.0813 | 2.1012 | 0.0782 | 3406 |
| 3090 | | | | | | | | | | | | | | | | | | |
| Phage- | T | E | L | A | — | K | — | L | — | — | — | N | — | — | 0.0924 | 2.098 | 0.0691 | 3407 |
| 3091 | | | | | | | | | | | | | | | | | | |
| Phage- | T | G | A | I | — | V | — | E | — | — | — | M | — | — | 0.0714 | 2.0976 | 0.0934 | 3408 |
| 3092 | | | | | | | | | | | | | | | | | | |
| Phage- | V | E | P | Y | — | K | — | A | — | — | — | T | — | — | 0.0602 | 2.0963 | 0.1098 | 3409 |
| 3093 | | | | | | | | | | | | | | | | | | |
| Phage- | G | — | M | V | — | K | — | A | — | — | — | Q | — | — | 0.0779 | 2.0957 | 0.0731 | 3410 |
| 3094 | | | | | | | | | | | | | | | | | | |
| Phage- | Y | D | F | R | — | I | — | S | — | — | — | M | — | I | 0.0545 | 2.0955 | 0.0693 | 3411 |
| 3095 | | | | | | | | | | | | | | | | | | |
| Phage- | Q | G | R | V | — | E | — | S | — | — | — | T | — | — | 0.0532 | 2.0942 | 0.0561 | 3412 |
| 3096 | | | | | | | | | | | | | | | | | | |
| Phage- | M | E | R | M | — | R | — | A | — | — | — | T | A | — | 0.0752 | 2.0931 | 0.1066 | 3413 |
| 3097 | | | | | | | | | | | | | | | | | | |
| Phage- | E | H | I | — | — | R | — | V | — | — | — | E | — | — | 0.0608 | 2.0912 | 0.1012 | 3414 |
| 3098 | | | | | | | | | | | | | | | | | | |
| Phage- | M | G | P | V | — | K | — | A | — | — | — | L | E | — | 0.0736 | 2.0911 | 0.1092 | 3415 |
| 3099 | | | | | | | | | | | | | | | | | | |
| Phage- | P | M | P | V | — | V | — | M | — | — | — | Y | — | — | 0.0643 | 2.0908 | 0.0842 | 3416 |
| 3100 | | | | | | | | | | | | | | | | | | |
| Phage- | — | L | P | Q | — | V | — | A | — | — | — | S | — | — | 0.0649 | 2.0904 | 0.0784 | 3417 |
| 3101 | | | | | | | | | | | | | | | | | | |
| Phage- | D | F | A | Y | — | K | — | E | — | — | — | V | — | — | 0.0804 | 2.0888 | 0.0844 | 3418 |
| 3102 | | | | | | | | | | | | | | | | | | |
| Phage- | G | E | I | — | — | R | — | A | — | — | — | Y | — | — | 0.0708 | 2.0877 | 0.119 | 3419 |
| 3103 | | | | | | | | | | | | | | | | | | |
| Phage- | G | E | — | — | — | R | — | A | — | — | — | S | — | — | 0.0603 | 2.0862 | 0.1104 | 3420 |
| 3104 | | | | | | | | | | | | | | | | | | |
| Phage- | Q | E | I | R | — | E | — | I | — | — | — | W | — | — | 0.074 | 2.0859 | 0.1472 | 3421 |
| 3105 | | | | | | | | | | | | | | | | | | |
| Phage- | N | P | M | Y | — | K | — | S | — | — | — | E | — | — | 0.0772 | 2.0845 | 0.0958 | 3422 |
| 3106 | | | | | | | | | | | | | | | | | | |
| Phage- | P | V | A | V | — | K | — | E | — | — | — | I | — | — | 0.0679 | 2.0841 | 0.1047 | 3423 |
| 3107 | | | | | | | | | | | | | | | | | | |
| Phage- | S | E | — | V | — | V | — | T | — | — | — | T | — | — | 0.0581 | 2.084 | 0.0805 | 3424 |
| 3108 | | | | | | | | | | | | | | | | | | |
| Phage- | V | E | A | M | — | K | — | W | — | — | — | E | — | L | 0.0535 | 2.0813 | 0.0719 | 3425 |
| 3109 | | | | | | | | | | | | | | | | | | |
| Phage- | V | V | Q | — | — | R | — | S | — | — | — | Q | — | — | 0.0796 | 2.0812 | 0.2814 | 3426 |
| 3110 | | | | | | | | | | | | | | | | | | |
| Phage- | — | E | I | M | — | M | — | R | — | — | — | D | — | — | 0.0721 | 2.0806 | 0.1008 | 3427 |
| 3111 | | | | | | | | | | | | | | | | | | |
| Phage- | E | P | Q | M | — | I | — | R | — | — | — | D | — | — | 0.0746 | 2.0803 | 0.1349 | 3428 |
| 3112 | | | | | | | | | | | | | | | | | | |
| Phage- | N | G | — | V | — | — | — | P | — | — | — | F | — | — | 0.0633 | 2.0803 | 0.0794 | 3429 |
| 3113 | | | | | | | | | | | | | | | | | | |
| Phage- | V | P | R | Y | — | V | — | Q | — | — | — | — | A | — | 0.0706 | 2.0782 | 0.0864 | 3430 |
| 3114 | | | | | | | | | | | | | | | | | | |
| Phage- | E | F | P | — | — | V | — | A | — | — | — | S | — | — | 0.064 | 2.0772 | 0.0859 | 3431 |
| 3115 | | | | | | | | | | | | | | | | | | |
| Phage- | F | H | I | M | — | — | — | E | — | — | — | L | — | — | 0.1044 | 2.0758 | 0.1291 | 3432 |
| 3116 | | | | | | | | | | | | | | | | | | |
| Phage- | S | G | A | — | — | V | — | L | — | — | — | T | — | — | 0.0625 | 2.0746 | 0.0979 | 3433 |
| 3117 | | | | | | | | | | | | | | | | | | |
| Phage- | L | E | S | M | — | I | — | P | — | — | — | S | — | L | 0.0593 | 2.0731 | 0.1094 | 3434 |
| 3118 | | | | | | | | | | | | | | | | | | |
| Phage- | D | Y | H | Y | — | E | — | A | — | — | — | F | — | — | 0.057 | 2.0723 | 0.0726 | 3435 |
| 3119 | | | | | | | | | | | | | | | | | | |
| Phage- | N | E | P | W | — | V | — | A | — | — | — | T | — | — | 0.0548 | 2.0717 | 0.0834 | 3436 |
| 3120 | | | | | | | | | | | | | | | | | | |
| Phage- | A | P | — | F | — | K | — | A | — | — | — | P | W | L | 0.0804 | 2.07 | 0.0695 | 3437 |
| 3121 | | | | | | | | | | | | | | | | | | |
| Phage- | G | M | I | Q | — | R | — | A | — | — | — | — | — | L | 0.0614 | 2.0687 | 0.0928 | 3438 |
| 3122 | | | | | | | | | | | | | | | | | | |
| Phage- | G | Y | P | — | — | I | — | E | — | — | — | — | — | — | 0.0707 | 2.0665 | 0.0968 | 3439 |
| 3123 | | | | | | | | | | | | | | | | | | |
| Phage- | S | M | H | W | — | E | — | M | — | — | — | — | — | — | 0.0705 | 2.0663 | 0.0809 | 3440 |
| 3124 | | | | | | | | | | | | | | | | | | |
| Phage- | V | N | — | K | — | V | — | S | — | — | — | S | — | — | 0.0746 | 2.0652 | 0.0739 | 3441 |
| 3125 | | | | | | | | | | | | | | | | | | |
| Phage- | F | E | I | — | — | R | — | L | — | — | — | S | — | — | 0.0627 | 2.0618 | 0.1105 | 3442 |
| 3126 | | | | | | | | | | | | | | | | | | |
| Phage- | L | D | K | R | — | V | — | V | — | — | — | E | — | — | 0.0594 | 2.0607 | 0.0828 | 3443 |
| 3127 | | | | | | | | | | | | | | | | | | |
| Phage- | L | E | L | A | — | K | — | E | — | — | — | V | — | — | 0.1017 | 2.0604 | 0.0801 | 3444 |
| 3128 | | | | | | | | | | | | | | | | | | |
| Phage- | G | H | Q | M | — | V | — | Q | — | — | — | D | A | — | 0.0718 | 2.0604 | 0.0607 | 3445 |
| 3129 | | | | | | | | | | | | | | | | | | |
| Phage- | S | V | G | Y | — | V | — | S | — | — | — | S | — | — | 0.1463 | 2.0596 | 0.075 | 3446 |
| 3130 | | | | | | | | | | | | | | | | | | |
| Phage- | D | — | — | R | — | V | — | S | — | — | — | E | — | — | 0.0734 | 2.0592 | 0.1003 | 3447 |
| 3131 | | | | | | | | | | | | | | | | | | |
| Phage- | T | H | I | I | — | K | — | A | — | — | — | D | A | W | 0.0524 | 2.0584 | 0.0691 | 3448 |
| 3132 | | | | | | | | | | | | | | | | | | |
| Phage- | Y | E | R | S | — | V | — | S | — | — | — | E | — | — | 0.0525 | 2.0576 | 0.066 | 3449 |
| 3133 | | | | | | | | | | | | | | | | | | |
| Phage- | A | E | R | — | — | V | — | A | — | — | — | L | N | L | 0.0558 | 2.0572 | 0.0543 | 3450 |
| 3134 | | | | | | | | | | | | | | | | | | |
| Phage- | H | Q | L | M | — | R | — | A | — | — | — | E | — | — | 0.0843 | 2.0563 | 0.1176 | 3451 |
| 3135 | | | | | | | | | | | | | | | | | | |
| Phage- | V | V | Q | I | — | K | — | S | — | — | — | E | — | — | 0.069 | 2.0558 | 0.1025 | 3452 |
| 3136 | | | | | | | | | | | | | | | | | | |
| Phage- | — | V | P | K | — | — | — | S | — | — | — | F | — | — | 0.0705 | 2.0557 | 0.0991 | 3453 |
| 3137 | | | | | | | | | | | | | | | | | | |
| Phage- | D | T | R | I | — | K | — | E | — | — | — | M | — | M | 0.0665 | 2.0555 | 0.1066 | 3454 |
| 3138 | | | | | | | | | | | | | | | | | | |
| Phage- | T | — | P | V | — | V | — | W | — | — | — | E | — | — | 0.0607 | 2.0548 | 0.0757 | 3455 |
| 3139 | | | | | | | | | | | | | | | | | | |
| Phage- | — | D | T | I | — | R | — | S | — | — | — | L | — | — | 0.0598 | 2.0538 | 0.0788 | 3456 |
| 3140 | | | | | | | | | | | | | | | | | | |
| Phage- | Y | E | R | V | — | E | — | L | — | — | — | T | — | — | 0.057 | 2.0533 | 0.0802 | 3457 |
| 3141 | | | | | | | | | | | | | | | | | | |
| Phage- | A | G | — | Y | — | T | — | M | — | — | — | T | — | — | 0.0617 | 2.0533 | 0.0775 | 3458 |
| 3142 | | | | | | | | | | | | | | | | | | |
| Phage- | S | V | P | — | — | I | — | E | — | — | — | V | — | — | 0.054 | 2.0525 | 0.1058 | 3459 |
| 3143 | | | | | | | | | | | | | | | | | | |
| Phage- | H | L | P | R | — | E | — | A | — | — | — | H | — | — | 0.0583 | 2.0512 | 0.0904 | 3460 |
| 3144 | | | | | | | | | | | | | | | | | | |
| Phage- | — | E | R | S | — | T | — | S | — | — | — | H | — | — | 0.0837 | 2.0511 | 0.072 | 3461 |
| 3145 | | | | | | | | | | | | | | | | | | |
| Phage- | G | P | I | M | — | — | — | R | — | — | — | E | — | — | 0.0575 | 2.0488 | 0.0807 | 3462 |
| 3146 | | | | | | | | | | | | | | | | | | |
| Phage- | F | E | M | F | — | I | — | A | — | — | — | S | — | L | 0.0782 | 2.0459 | 0.0821 | 3463 |
| 3147 | | | | | | | | | | | | | | | | | | |
| Phage- | A | E | Y | M | — | I | — | S | — | — | — | H | — | M | 0.0963 | 2.0454 | 0.0861 | 3464 |
| 3148 | | | | | | | | | | | | | | | | | | |
| Phage- | L | L | P | A | — | K | — | T | — | — | — | T | — | L | 0.1061 | 2.0441 | 0.1352 | 3465 |
| 3149 | | | | | | | | | | | | | | | | | | |
| Phage- | D | — | P | — | — | K | — | Q | — | — | — | S | — | — | 0.1047 | 2.0428 | 0.0854 | 3466 |
| 3150 | | | | | | | | | | | | | | | | | | |
| Phage- | F | P | R | — | — | E | — | L | — | — | — | K | — | — | 0.0643 | 2.0427 | 0.0987 | 3467 |
| 3151 | | | | | | | | | | | | | | | | | | |
| Phage- | W | D | Q | W | — | V | — | K | — | — | — | Q | — | — | 0.1349 | 2.0387 | 0.1965 | 3468 |
| 3152 | | | | | | | | | | | | | | | | | | |
| Phage- | D | M | S | — | — | K | — | P | — | — | — | F | — | — | 0.0618 | 2.0385 | 0.0792 | 3469 |
| 3153 | | | | | | | | | | | | | | | | | | |
| Phage- | P | W | I | V | — | K | — | A | — | — | — | T | — | — | 0.0694 | 2.038 | 0.1184 | 3470 |
| 3154 | | | | | | | | | | | | | | | | | | |
| Phage- | V | S | S | M | — | K | — | A | — | — | — | S | — | — | 0.07 | 2.0376 | 0.0629 | 3471 |
| 3155 | | | | | | | | | | | | | | | | | | |
| Phage- | — | Q | — | M | — | R | — | A | — | — | — | S | — | — | 0.0644 | 2.037 | 0.0959 | 3472 |
| 3156 | | | | | | | | | | | | | | | | | | |
| Phage- | Q | M | P | M | — | R | — | A | — | — | — | M | — | — | 0.0985 | 2.0364 | 0.1055 | 3473 |
| 3157 | | | | | | | | | | | | | | | | | | |
| Phage- | — | V | P | Y | — | M | — | Q | — | — | — | S | — | — | 0.0809 | 2.0359 | 0.107 | 3474 |
| 3158 | | | | | | | | | | | | | | | | | | |
| Phage- | W | M | A | M | — | K | — | V | — | — | — | E | — | — | 0.1183 | 2.035 | 0.163 | 3475 |
| 3159 | | | | | | | | | | | | | | | | | | |
| Phage- | L | — | L | R | — | E | — | S | — | — | — | L | — | — | 0.0726 | 2.0347 | 0.112 | 3476 |
| 3160 | | | | | | | | | | | | | | | | | | |
| Phage- | N | H | — | Q | — | V | — | — | — | — | — | S | — | — | 0.0602 | 2.0312 | 0.1384 | 3477 |
| 3161 | | | | | | | | | | | | | | | | | | |
| Phage- | E | T | A | — | — | R | — | L | — | — | — | D | W | L | 0.0716 | 2.0305 | 0.0692 | 3478 |
| 3162 | | | | | | | | | | | | | | | | | | |
| Phage- | D | I | H | — | — | T | — | — | — | — | — | S | — | — | 0.0693 | 2.0303 | 0.0738 | 3479 |
| 3163 | | | | | | | | | | | | | | | | | | |
| Phage- | M | D | K | I | — | S | — | M | — | — | — | L | — | — | 0.0618 | 2.0273 | 0.0702 | 3480 |
| 3164 | | | | | | | | | | | | | | | | | | |
| Phage- | N | V | H | Y | — | V | — | A | — | — | — | N | — | — | 0.0714 | 2.0272 | 0.1629 | 3481 |
| 3165 | | | | | | | | | | | | | | | | | | |
| Phage- | L | V | R | V | — | T | — | M | — | — | — | Q | — | — | 0.0649 | 2.0268 | 0.058 | 3482 |
| 3166 | | | | | | | | | | | | | | | | | | |
| Phage- | T | Y | P | K | — | I | — | P | — | — | — | T | — | — | 0.0641 | 2.0258 | 0.1147 | 3483 |
| 3167 | | | | | | | | | | | | | | | | | | |
| Phage- | A | D | W | — | — | R | — | A | — | — | — | E | — | L | 0.0527 | 2.0229 | 0.0589 | 3484 |
| 3168 | | | | | | | | | | | | | | | | | | |
| Phage- | V | E | K | — | — | K | — | E | — | — | — | M | — | — | 0.063 | 2.0223 | 0.0909 | 3485 |
| 3169 | | | | | | | | | | | | | | | | | | |
| Phage- | E | V | K | F | — | K | — | A | — | — | — | T | — | — | 0.0515 | 2.0221 | 0.0863 | 3486 |
| 3170 | | | | | | | | | | | | | | | | | | |
| Phage- | G | E | — | F | — | I | — | A | — | — | — | — | — | — | 0.0802 | 2.0217 | 0.1072 | 3487 |
| 3171 | | | | | | | | | | | | | | | | | | |
| Phage- | A | N | L | — | — | I | — | — | — | — | — | S | — | — | 0.0654 | 2.0213 | 0.0969 | 3488 |
| 3172 | | | | | | | | | | | | | | | | | | |
| Phage- | M | D | R | M | — | V | — | E | — | — | — | Y | V | — | 0.0626 | 2.021 | 0.0741 | 3489 |
| 3173 | | | | | | | | | | | | | | | | | | |
| Phage- | V | T | A | K | — | V | — | E | — | — | — | T | — | — | 0.0672 | 2.0207 | 0.0968 | 3490 |
| 3174 | | | | | | | | | | | | | | | | | | |
| Phage- | Y | P | S | M | — | K | — | L | — | — | — | Q | — | — | 0.0702 | 2.0202 | 0.1109 | 3491 |
| 3175 | | | | | | | | | | | | | | | | | | |
| Phage- | V | T | R | M | — | V | — | E | — | — | — | S | — | I | 0.0533 | 2.0193 | 0.0628 | 3492 |
| 3176 | | | | | | | | | | | | | | | | | | |
| Phage- | Y | E | P | V | — | R | — | A | — | — | — | I | — | — | 0.0508 | 2.0181 | 0.0622 | 3493 |
| 3177 | | | | | | | | | | | | | | | | | | |
| Phage- | Y | E | Q | A | — | — | — | P | — | — | — | L | — | — | 0.0619 | 2.0168 | 0.0671 | 3494 |
| 3178 | | | | | | | | | | | | | | | | | | |
| Phage- | Y | M | M | I | — | R | — | A | — | — | — | S | — | L | 0.1033 | 2.0136 | 0.116 | 3495 |
| 3179 | | | | | | | | | | | | | | | | | | |
| Phage- | H | E | R | E | — | I | — | R | — | — | — | T | — | — | 0.0599 | 2.0135 | 0.065 | 3496 |
| 3180 | | | | | | | | | | | | | | | | | | |
| Phage- | G | V | S | M | — | I | — | A | — | — | — | Q | — | — | 0.0589 | 2.0107 | 0.0737 | 3497 |
| 3181 | | | | | | | | | | | | | | | | | | |
| Phage- | — | Q | I | — | — | T | — | T | — | — | — | V | — | — | 0.0622 | 2.0103 | 0.0781 | 3498 |
| 3182 | | | | | | | | | | | | | | | | | | |
| Phage- | E | G | A | I | — | K | — | E | — | — | — | K | — | — | 0.0549 | 2.009 | 0.0737 | 3499 |
| 3183 | | | | | | | | | | | | | | | | | | |
| Phage- | M | V | R | — | — | E | — | E | — | — | — | T | — | — | 0.0891 | 2.0074 | 0.116 | 3500 |
| 3184 | | | | | | | | | | | | | | | | | | |
| Phage- | — | E | K | W | — | F | — | M | — | — | — | E | — | — | 0.0793 | 2.0072 | 0.0806 | 3501 |
| 3185 | | | | | | | | | | | | | | | | | | |
| Phage- | V | S | E | — | — | R | — | A | — | — | — | H | — | — | 0.0756 | 2.0046 | 0.082 | 3502 |
| 3186 | | | | | | | | | | | | | | | | | | |
| Phage- | A | — | P | M | — | I | — | A | — | — | — | H | — | — | 0.0855 | 2.0041 | 0.1365 | 3503 |
| 3187 | | | | | | | | | | | | | | | | | | |
| Phage- | D | M | R | V | — | K | — | E | — | — | — | T | — | L | 0.0732 | 2.0038 | 0.1087 | 3504 |
| 3188 | | | | | | | | | | | | | | | | | | |
| Phage- | L | E | A | — | — | V | — | R | — | — | — | E | — | — | 0.0973 | 2.0024 | 0.096 | 3505 |
| 3189 | | | | | | | | | | | | | | | | | | |
| Phage- | A | D | L | R | — | V | — | E | — | — | — | M | — | I | 0.0672 | 2.0023 | 0.0654 | 3506 |
| 3190 | | | | | | | | | | | | | | | | | | |
| Phage- | Q | H | W | Q | — | V | — | R | — | — | — | D | — | — | 0.0611 | 2.0001 | 0.1035 | 3507 |
| 3191 | | | | | | | | | | | | | | | | | | |
| Phage- | E | V | — | M | — | R | — | L | — | — | — | S | — | — | 0.0798 | 1.9976 | 0.0925 | 3508 |
| 3192 | | | | | | | | | | | | | | | | | | |
| Phage- | G | E | A | V | — | I | — | R | — | — | — | D | — | — | 0.059 | 1.9946 | 0.0937 | 3509 |
| 3193 | | | | | | | | | | | | | | | | | | |
| Phage- | D | M | P | — | — | I | — | A | — | — | — | — | — | — | 0.0522 | 1.994 | 0.0615 | 3510 |
| 3194 | | | | | | | | | | | | | | | | | | |
| Phage- | H | E | H | F | — | R | — | L | — | — | — | T | — | L | 0.0576 | 1.99 | 0.0753 | 3511 |
| 3195 | | | | | | | | | | | | | | | | | | |
| Phage- | V | E | — | R | — | V | — | A | — | — | — | T | — | — | 0.0782 | 1.9893 | 0.0941 | 3512 |
| 3196 | | | | | | | | | | | | | | | | | | |
| Phage- | L | S | P | M | — | V | — | Q | — | — | — | S | — | — | 0.0619 | 1.9861 | 0.1188 | 3513 |
| 3197 | | | | | | | | | | | | | | | | | | |
| Phage- | F | D | R | V | — | K | — | E | — | — | — | N | — | L | 0.0704 | 1.9854 | 0.0655 | 3514 |
| 3198 | | | | | | | | | | | | | | | | | | |
| Phage- | V | T | — | R | — | E | — | A | — | — | — | M | — | — | 0.0603 | 1.9849 | 0.0999 | 3515 |
| 3199 | | | | | | | | | | | | | | | | | | |
| Phage- | V | T | P | — | — | V | — | K | — | — | — | E | L | — | 0.0582 | 1.9822 | 0.0878 | 3516 |
| 3200 | | | | | | | | | | | | | | | | | | |
| Phage- | V | V | P | — | — | I | — | Q | — | — | — | — | V | — | 0.0667 | 1.9806 | 0.0757 | 3517 |
| 3201 | | | | | | | | | | | | | | | | | | |
| Phage- | V | V | — | S | — | I | — | T | — | — | — | V | — | — | 0.0698 | 1.9781 | 0.1034 | 3518 |
| 3202 | | | | | | | | | | | | | | | | | | |
| Phage- | V | S | — | R | — | E | — | E | — | — | — | M | — | — | 0.057 | 1.9771 | 0.0741 | 3519 |
| 3203 | | | | | | | | | | | | | | | | | | |
| Phage- | L | E | R | — | — | — | — | S | — | — | — | T | — | — | 0.0545 | 1.9768 | 0.0739 | 3520 |
| 3204 | | | | | | | | | | | | | | | | | | |
| Phage- | V | V | K | I | — | — | — | D | — | — | — | W | — | — | 0.061 | 1.9767 | 0.0655 | 3521 |
| 3205 | | | | | | | | | | | | | | | | | | |
| Phage- | T | V | — | K | — | V | — | E | — | — | — | E | W | L | 0.0639 | 1.9765 | 0.0606 | 3522 |
| 3206 | | | | | | | | | | | | | | | | | | |
| Phage- | H | D | P | A | — | V | — | S | — | — | — | F | — | — | 0.0525 | 1.9763 | 0.0668 | 3523 |
| 3207 | | | | | | | | | | | | | | | | | | |
| Phage- | P | F | A | Y | — | V | — | Q | — | — | — | E | A | — | 0.0921 | 1.9754 | 0.0639 | 3524 |
| 3208 | | | | | | | | | | | | | | | | | | |
| Phage- | D | H | H | I | — | R | — | A | — | — | — | M | — | L | 0.0579 | 1.9741 | 0.0876 | 3525 |
| 3209 | | | | | | | | | | | | | | | | | | |
| Phage- | D | T | P | K | — | I | — | Q | — | — | — | V | — | — | 0.0604 | 1.9735 | 0.0677 | 3526 |
| 3210 | | | | | | | | | | | | | | | | | | |
| Phage- | P | E | A | M | — | R | — | T | — | — | — | — | — | — | 0.0867 | 1.9729 | 0.0674 | 3527 |
| 3211 | | | | | | | | | | | | | | | | | | |
| Phage- | T | V | P | — | — | K | — | L | — | — | — | Y | — | — | 0.11 | 1.9725 | 0.1836 | 3528 |
| 3212 | | | | | | | | | | | | | | | | | | |
| Phage- | A | I | A | M | — | V | — | S | — | — | — | T | — | L | 0.0529 | 1.9723 | 0.0711 | 3529 |
| 3213 | | | | | | | | | | | | | | | | | | |
| Phage- | M | — | P | Y | — | V | — | S | — | — | — | W | — | — | 0.1009 | 1.9714 | 0.1453 | 3530 |
| 3214 | | | | | | | | | | | | | | | | | | |
| Phage- | L | D | L | R | — | — | — | P | — | — | — | M | — | — | 0.0555 | 1.9713 | 0.0778 | 3531 |
| 3215 | | | | | | | | | | | | | | | | | | |
| Phage- | V | E | P | — | — | V | — | S | — | — | — | Q | — | I | 0.0599 | 1.9709 | 0.076 | 3532 |
| 3216 | | | | | | | | | | | | | | | | | | |
| Phage- | — | E | L | F | — | K | — | A | — | — | — | D | — | — | 0.073 | 1.969 | 0.1108 | 3533 |
| 3217 | | | | | | | | | | | | | | | | | | |
| Phage- | D | V | H | — | — | K | — | S | — | — | — | — | — | — | 0.0524 | 1.9636 | 0.0706 | 3534 |
| 3218 | | | | | | | | | | | | | | | | | | |
| Phage- | D | Q | — | M | — | K | — | I | — | — | — | E | — | — | 0.0579 | 1.9623 | 0.0928 | 3535 |
| 3219 | | | | | | | | | | | | | | | | | | |
| Phage- | — | p | R | A | — | I | — | A | — | — | — | Q | — | — | 0.0662 | 1.9606 | 0.1013 | 3536 |
| 3220 | | | | | | | | | | | | | | | | | | |
| Phage- | L | Q | I | — | — | K | — | Q | — | — | — | E | — | L | 0.066 | 1.959 | 0.0882 | 3537 |
| 3221 | | | | | | | | | | | | | | | | | | |
| Phage- | M | E | L | K | — | V | — | K | — | — | — | — | — | — | 0.0524 | 1.957 | 0.0684 | 3538 |
| 3222 | | | | | | | | | | | | | | | | | | |
| Phage- | P | Q | W | M | — | K | — | E | — | — | — | N | — | — | 0.0602 | 1.9567 | 0.0807 | 3539 |
| 3223 | | | | | | | | | | | | | | | | | | |
| Phage- | — | T | A | — | — | K | — | A | — | — | — | D | — | — | 0.0632 | 1.9559 | 0.1087 | 3540 |
| 3224 | | | | | | | | | | | | | | | | | | |
| Phage- | G | E | — | — | — | R | — | I | — | — | — | N | — | — | 0.0532 | 1.9531 | 0.1231 | 3541 |
| 3225 | | | | | | | | | | | | | | | | | | |
| Phage- | L | T | — | — | — | E | — | A | — | — | — | V | — | — | 0.0508 | 1.9521 | 0.0827 | 3542 |
| 3226 | | | | | | | | | | | | | | | | | | |
| Phage- | P | I | — | K | — | S | — | A | — | — | — | S | — | — | 0.0577 | 1.9512 | 0.0757 | 3543 |
| 3227 | | | | | | | | | | | | | | | | | | |
| Phage- | V | G | H | V | — | L | — | P | — | — | — | T | — | — | 0.0556 | 1.951 | 0.0697 | 3544 |
| 3228 | | | | | | | | | | | | | | | | | | |
| Phage- | — | T | P | — | — | S | — | A | — | — | — | Q | — | L | 0.0555 | 1.9506 | 0.055 | 3545 |
| 3229 | | | | | | | | | | | | | | | | | | |
| Phage- | L | G | I | Y | — | V | — | E | — | — | — | Q | — | — | 0.0756 | 1.9498 | 0.0818 | 3546 |
| 3230 | | | | | | | | | | | | | | | | | | |
| Phage- | V | T | A | Y | — | V | — | P | — | — | — | — | V | — | 0.0694 | 1.9486 | 0.1022 | 3547 |
| 3231 | | | | | | | | | | | | | | | | | | |
| Phage- | — | V | K | A | — | K | — | E | — | — | — | V | — | — | 0.0578 | 1.9476 | 0.0754 | 3548 |
| 3232 | | | | | | | | | | | | | | | | | | |
| Phage- | P | Y | A | M | — | E | — | A | — | — | — | S | — | — | 0.0598 | 1.9475 | 0.0697 | 3549 |
| 3233 | | | | | | | | | | | | | | | | | | |
| Phage- | D | G | I | R | — | V | — | — | — | — | — | V | — | — | 0.0641 | 1.9463 | 0.0969 | 3550 |
| 3234 | | | | | | | | | | | | | | | | | | |
| Phage- | V | E | R | — | — | M | — | E | — | — | — | L | — | — | 0.0762 | 1.946 | 0.1264 | 3551 |
| 3235 | | | | | | | | | | | | | | | | | | |
| Phage- | P | L | P | M | — | V | — | A | — | — | — | — | — | — | 0.0764 | 1.9454 | 0.1327 | 3552 |
| 3236 | | | | | | | | | | | | | | | | | | |
| Phage- | E | Y | H | K | — | I | — | E | — | — | — | — | — | — | 0.057 | 1.9421 | 0.0924 | 3553 |
| 3237 | | | | | | | | | | | | | | | | | | |
| Phage- | W | E | H | R | — | T | — | E | — | — | — | T | — | — | 0.0661 | 1.9396 | 0.0937 | 3554 |
| 3238 | | | | | | | | | | | | | | | | | | |
| Phage- | D | S | — | — | — | V | — | S | — | — | — | H | — | — | 0.0596 | 1.9394 | 0.0834 | 3555 |
| 3239 | | | | | | | | | | | | | | | | | | |
| Phage- | G | F | R | I | — | E | — | E | — | — | — | V | — | — | 0.0694 | 1.9392 | 0.0629 | 3556 |
| 3240 | | | | | | | | | | | | | | | | | | |
| Phage- | M | V | E | R | — | I | — | V | — | — | — | E | — | — | 0.065 | 1.9385 | 0.0937 | 3557 |
| 3241 | | | | | | | | | | | | | | | | | | |
| Phage- | E | T | S | M | — | R | — | S | — | — | — | E | — | — | 0.0569 | 1.9377 | 0.0918 | 3558 |
| 3242 | | | | | | | | | | | | | | | | | | |
| Phage- | V | N | — | R | — | E | — | S | — | — | — | T | — | L | 0.0536 | 1.9373 | 0.0801 | 3559 |
| 3243 | | | | | | | | | | | | | | | | | | |
| Phage- | L | S | A | M | — | V | — | P | — | — | — | T | — | — | 0.0741 | 1.9356 | 0.1379 | 3560 |
| 3244 | | | | | | | | | | | | | | | | | | |
| Phage- | N | I | L | R | — | E | — | M | — | — | — | T | — | — | 0.0902 | 1.9355 | 0.0977 | 3561 |
| 3245 | | | | | | | | | | | | | | | | | | |
| Phage- | Q | D | I | V | — | R | — | S | — | — | — | H | — | — | 0.0579 | 1.9354 | 0.0762 | 3562 |
| 3246 | | | | | | | | | | | | | | | | | | |
| Phage- | G | L | A | — | — | K | — | A | — | — | — | Q | — | — | 0.0561 | 1.9334 | 0.0706 | 3563 |
| 3247 | | | | | | | | | | | | | | | | | | |
| Phage- | V | T | H | S | — | V | — | A | — | — | — | V | — | L | 0.0881 | 1.9313 | 0.091 | 3564 |
| 3248 | | | | | | | | | | | | | | | | | | |
| Phage- | M | M | H | N | — | I | — | Y | — | — | — | — | — | — | 0.0893 | 1.9301 | 0.1006 | 3565 |
| 3249 | | | | | | | | | | | | | | | | | | |
| Phage- | N | V | P | V | — | I | — | D | — | — | — | V | — | — | 0.0706 | 1.9298 | 0.0851 | 3566 |
| 3250 | | | | | | | | | | | | | | | | | | |
| Phage- | G | V | I | A | — | K | — | H | — | — | — | T | — | — | 0.0561 | 1.9297 | 0.085 | 3567 |
| 3251 | | | | | | | | | | | | | | | | | | |
| Phage- | M | P | M | H | — | V | — | S | — | — | — | E | — | — | 0.1007 | 1.9272 | 0.0901 | 3568 |
| 3252 | | | | | | | | | | | | | | | | | | |
| Phage- | S | I | P | K | — | M | — | E | — | — | — | I | — | L | 0.0571 | 1.9261 | 0.0587 | 3569 |
| 3253 | | | | | | | | | | | | | | | | | | |
| Phage- | Y | G | A | — | — | V | — | A | — | — | — | S | — | — | 0.0548 | 1.9257 | 0.0866 | 3570 |
| 3254 | | | | | | | | | | | | | | | | | | |
| Phage- | — | V | P | W | — | K | — | A | — | — | — | — | V | S | 0.09 | 1.9251 | 0.1196 | 3571 |
| 3255 | | | | | | | | | | | | | | | | | | |
| Phage- | D | G | F | I | — | R | — | E | — | — | — | Q | W | L | 0.0586 | 1.9251 | 0.0653 | 3572 |
| 3256 | | | | | | | | | | | | | | | | | | |
| Phage- | L | G | N | — | — | V | — | P | — | — | — | Q | — | — | 0.0676 | 1.9239 | 0.2409 | 3573 |
| 3257 | | | | | | | | | | | | | | | | | | |
| Phage- | Q | P | G | Y | — | V | — | Q | — | — | — | M | — | — | 0.0607 | 1.9232 | 0.0948 | 3574 |
| 3258 | | | | | | | | | | | | | | | | | | |
| Phage- | A | E | M | R | — | I | — | Q | — | — | — | S | — | L | 0.0699 | 1.9205 | 0.1384 | 3575 |
| 3259 | | | | | | | | | | | | | | | | | | |
| Phage- | E | N | — | R | — | E | — | M | — | — | — | K | — | — | 0.0549 | 1.919 | 0.0648 | 3576 |
| 3260 | | | | | | | | | | | | | | | | | | |
| Phage- | P | T | P | V | — | K | — | G | — | — | — | D | L | — | 0.0732 | 1.9174 | 0.0845 | 3577 |
| 3261 | | | | | | | | | | | | | | | | | | |
| Phage- | V | S | I | V | — | V | — | E | — | — | — | S | — | — | 0.0511 | 1.9155 | 0.0721 | 3578 |
| 3262 | | | | | | | | | | | | | | | | | | |
| Phage- | F | F | K | M | — | I | — | E | — | — | — | S | — | — | 0.1149 | 1.9131 | 0.205 | 3579 |
| 3263 | | | | | | | | | | | | | | | | | | |
| Phage- | E | E | N | F | — | V | — | K | — | — | — | E | — | — | 0.0793 | 1.911 | 0.1256 | 3580 |
| 3264 | | | | | | | | | | | | | | | | | | |
| Phage- | E | I | M | — | — | K | — | E | — | — | — | Y | — | — | 0.0764 | 1.9092 | 0.0998 | 3581 |
| 3265 | | | | | | | | | | | | | | | | | | |
| Phage- | E | Y | Q | V | — | K | — | T | — | — | — | T | — | — | 0.0579 | 1.909 | 0.0904 | 3582 |
| 3266 | | | | | | | | | | | | | | | | | | |
| Phage- | L | P | Y | M | — | T | — | I | — | — | — | I | — | — | 0.0953 | 1.9089 | 0.1431 | 3583 |
| 3267 | | | | | | | | | | | | | | | | | | |
| Phage- | V | E | — | K | — | V | — | E | — | — | — | T | — | — | 0.06 | 1.9076 | 0.0868 | 3584 |
| 3268 | | | | | | | | | | | | | | | | | | |
| Phage- | — | S | P | R | — | E | — | L | — | — | — | T | — | L | 0.0619 | 1.9051 | 0.0836 | 3585 |
| 3269 | | | | | | | | | | | | | | | | | | |
| Phage- | L | D | P | M | — | V | — | S | — | — | — | W | — | — | 0.0731 | 1.9033 | 0.0992 | 3586 |
| 3270 | | | | | | | | | | | | | | | | | | |
| Phage- | G | G | — | — | — | — | — | A | — | — | — | I | — | — | 0.143 | 1.9027 | 0.0804 | 3587 |
| 3271 | | | | | | | | | | | | | | | | | | |
| Phage- | — | N | A | W | — | I | — | A | — | — | — | E | — | — | 0.064 | 1.9022 | 0.1365 | 3588 |
| 3272 | | | | | | | | | | | | | | | | | | |
| Phage- | S | M | G | — | — | K | — | P | — | — | — | — | — | — | 0.0713 | 1.9022 | 0.0756 | 3589 |
| 3273 | | | | | | | | | | | | | | | | | | |
| Phage- | M | V | — | R | — | E | — | E | — | — | — | I | — | — | 0.0784 | 1.9013 | 0.0649 | 3590 |
| 3274 | | | | | | | | | | | | | | | | | | |
| Phage- | F | D | R | I | — | K | — | A | — | — | — | I | E | — | 0.0556 | 1.9002 | 0.0573 | 3591 |
| 3275 | | | | | | | | | | | | | | | | | | |
| Phage- | E | F | Q | M | — | V | — | E | — | — | — | T | — | L | 0.0697 | 1.8994 | 0.0838 | 3592 |
| 3276 | | | | | | | | | | | | | | | | | | |
| Phage- | P | — | R | M | — | V | — | A | — | — | — | S | V | L | 0.0665 | 1.8984 | 0.0732 | 3593 |
| 3277 | | | | | | | | | | | | | | | | | | |
| Phage- | L | P | P | M | — | I | — | Q | — | — | — | M | H | — | 0.058 | 1.8983 | 0.0592 | 3594 |
| 3278 | | | | | | | | | | | | | | | | | | |
| Phage- | G | E | P | W | — | I | — | S | — | — | — | Q | — | — | 0.0904 | 1.8974 | 0.091 | 3595 |
| 3279 | | | | | | | | | | | | | | | | | | |
| Phage- | W | N | H | V | — | I | — | T | — | — | — | T | — | M | 0.077 | 1.8962 | 0.0817 | 3596 |
| 3280 | | | | | | | | | | | | | | | | | | |
| Phage- | V | E | R | M | — | K | — | E | — | — | — | F | — | — | 0.0602 | 1.8959 | 0.1021 | 3597 |
| 3281 | | | | | | | | | | | | | | | | | | |
| Phage- | M | E | H | W | — | V | — | A | — | — | — | S | — | — | 0.07 | 1.8948 | 0.2641 | 3598 |
| 3282 | | | | | | | | | | | | | | | | | | |
| Phage- | F | D | H | — | — | I | — | V | — | — | — | K | — | — | 0.0727 | 1.8946 | 0.0828 | 3599 |
| 3283 | | | | | | | | | | | | | | | | | | |
| Phage- | E | L | Q | R | — | I | — | Q | — | — | — | V | — | L | 0.0624 | 1.8943 | 0.0731 | 3600 |
| 3284 | | | | | | | | | | | | | | | | | | |
| Phage- | F | D | T | Y | — | K | — | A | — | — | — | E | — | — | 0.0647 | 1.8931 | 0.0818 | 3601 |
| 3285 | | | | | | | | | | | | | | | | | | |
| Phage- | P | T | L | R | — | E | — | L | — | — | — | — | — | — | 0.0526 | 1.8911 | 0.0911 | 3602 |
| 3286 | | | | | | | | | | | | | | | | | | |
| Phage- | E | S | L | — | — | K | — | K | — | — | — | D | W | L | 0.0671 | 1.8897 | 0.0996 | 3603 |
| 3287 | | | | | | | | | | | | | | | | | | |
| Phage- | V | D | A | Y | — | K | — | A | — | — | — | L | — | — | 0.0853 | 1.8896 | 0.0998 | 3604 |
| 3288 | | | | | | | | | | | | | | | | | | |
| Phage- | E | D | Y | V | — | R | — | T | — | — | — | V | — | — | 0.0712 | 1.8886 | 0.0755 | 3605 |
| 3289 | | | | | | | | | | | | | | | | | | |
| Phage- | M | D | — | — | — | R | — | E | — | — | — | T | — | — | 0.0802 | 1.8881 | 0.0971 | 3606 |
| 3290 | | | | | | | | | | | | | | | | | | |
| Phage- | P | I | P | R | — | — | — | L | — | — | — | H | — | — | 0.083 | 1.8867 | 0.085 | 3607 |
| 3291 | | | | | | | | | | | | | | | | | | |
| Phage- | Y | E | M | R | — | V | — | D | — | — | — | V | — | L | 0.0835 | 1.8864 | 0.0906 | 3608 |
| 3292 | | | | | | | | | | | | | | | | | | |
| Phage- | E | T | L | M | — | I | — | — | — | — | — | D | — | — | 0.0679 | 1.8861 | 0.0837 | 3609 |
| 3293 | | | | | | | | | | | | | | | | | | |
| Phage- | T | D | — | M | — | V | — | Q | — | — | — | D | — | — | 0.0718 | 1.886 | 0.1065 | 3610 |
| 3294 | | | | | | | | | | | | | | | | | | |
| Phage- | T | E | — | M | — | R | — | L | — | — | — | — | — | — | 0.057 | 1.8853 | 0.1146 | 3611 |
| 3295 | | | | | | | | | | | | | | | | | | |
| Phage- | V | V | I | Q | — | — | — | A | — | — | — | D | — | — | 0.0539 | 1.8845 | 0.063 | 3612 |
| 3296 | | | | | | | | | | | | | | | | | | |
| Phage- | V | — | — | M | — | R | — | A | — | — | — | Y | — | — | 0.0569 | 1.8842 | 0.0851 | 3613 |
| 3297 | | | | | | | | | | | | | | | | | | |
| Phage- | E | T | F | K | — | V | — | E | — | — | — | T | — | L | 0.0531 | 1.8838 | 0.0673 | 3614 |
| 3298 | | | | | | | | | | | | | | | | | | |
| Phage- | H | D | A | M | — | K | — | A | — | — | — | — | — | — | 0.068 | 1.8802 | 0.0896 | 3615 |
| 3299 | | | | | | | | | | | | | | | | | | |
| Phage- | V | E | S | M | — | K | — | A | — | — | — | I | — | — | 0.059 | 1.8781 | 0.0872 | 3616 |
| 3300 | | | | | | | | | | | | | | | | | | |
| Phage- | M | P | P | V | — | I | — | R | — | — | — | E | — | — | 0.0675 | 1.8769 | 0.1112 | 3617 |
| 3301 | | | | | | | | | | | | | | | | | | |
| Phage- | M | L | L | Q | — | K | — | S | — | — | — | — | — | — | 0.0793 | 1.8738 | 0.0974 | 3618 |
| 3302 | | | | | | | | | | | | | | | | | | |
| Phage- | G | W | P | V | — | V | — | P | — | — | — | D | — | — | 0.0777 | 1.8738 | 0.0776 | 3619 |
| 3303 | | | | | | | | | | | | | | | | | | |
| Phage- | E | H | Y | A | — | K | — | E | — | — | — | — | — | — | 0.0639 | 1.8735 | 0.0803 | 3620 |
| 3304 | | | | | | | | | | | | | | | | | | |
| Phage- | H | S | A | — | — | I | — | S | — | — | — | Q | — | — | 0.053 | 1.8731 | 0.0729 | 3621 |
| 3305 | | | | | | | | | | | | | | | | | | |
| Phage- | V | D | R | F | — | E | — | I | — | — | — | I | — | — | 0.0538 | 1.8721 | 0.0694 | 3622 |
| 3306 | | | | | | | | | | | | | | | | | | |
| Phage- | E | M | F | R | — | T | — | E | — | — | — | — | — | L | 0.1077 | 1.872 | 0.1871 | 3623 |
| 3307 | | | | | | | | | | | | | | | | | | |
| Phage- | N | W | K | V | — | E | — | T | — | — | — | E | — | — | 0.0694 | 1.8708 | 0.0575 | 3624 |
| 3308 | | | | | | | | | | | | | | | | | | |
| Phage- | V | E | R | A | — | V | — | A | — | — | — | T | — | — | 0.0573 | 1.8706 | 0.0832 | 3625 |
| 3309 | | | | | | | | | | | | | | | | | | |
| Phage- | — | E | K | — | — | K | — | Q | — | — | — | E | — | — | 0.073 | 1.8685 | 0.1294 | 3626 |
| 3310 | | | | | | | | | | | | | | | | | | |
| Phage- | L | E | F | R | — | V | — | E | — | — | — | V | — | — | 0.0744 | 1.8656 | 0.1107 | 3627 |
| 3311 | | | | | | | | | | | | | | | | | | |
| Phage- | D | L | R | W | — | I | — | E | — | — | — | I | — | I | 0.0542 | 1.8643 | 0.0809 | 3628 |
| 3312 | | | | | | | | | | | | | | | | | | |
| Phage- | A | E | H | V | — | R | — | A | — | — | — | T | V | — | 0.0609 | 1.8629 | 0.0585 | 3629 |
| 3313 | | | | | | | | | | | | | | | | | | |
| Phage- | L | L | E | M | — | K | — | H | — | — | — | V | — | — | 0.0657 | 1.8623 | 0.085 | 3630 |
| 3314 | | | | | | | | | | | | | | | | | | |
| Phage- | V | E | I | A | — | K | — | Q | — | — | — | L | — | — | 0.0659 | 1.862 | 0.0766 | 3631 |
| 3315 | | | | | | | | | | | | | | | | | | |
| Phage- | Q | E | F | R | — | V | — | E | — | — | — | I | — | L | 0.0575 | 1.8604 | 0.0577 | 3632 |
| 3316 | | | | | | | | | | | | | | | | | | |
| Phage- | S | S | — | M | — | I | — | Q | — | — | — | S | — | — | 0.0642 | 1.8596 | 0.0899 | 3633 |
| 3317 | | | | | | | | | | | | | | | | | | |
| Phage- | L | P | I | M | — | K | — | Q | — | — | — | N | — | — | 0.0689 | 1.8559 | 0.08 | 3634 |
| 3318 | | | | | | | | | | | | | | | | | | |
| Phage- | L | Q | — | M | — | V | — | R | — | — | — | E | — | — | 0.0541 | 1.8558 | 0.1037 | 3635 |
| 3319 | | | | | | | | | | | | | | | | | | |
| Phage- | V | G | H | V | — | I | — | S | — | — | — | D | — | M | 0.058 | 1.8555 | 0.0683 | 3636 |
| 3320 | | | | | | | | | | | | | | | | | | |
| Phage- | W | V | I | K | — | V | — | S | — | — | — | — | — | L | 0.0816 | 1.8549 | 0.0907 | 3637 |
| 3321 | | | | | | | | | | | | | | | | | | |
| Phage- | — | M | P | K | — | V | — | S | — | — | — | T | — | L | 0.1277 | 1.8543 | 0.1529 | 3638 |
| 3322 | | | | | | | | | | | | | | | | | | |
| Phage- | A | G | S | M | — | T | — | M | — | — | — | T | — | — | 0.0786 | 1.8531 | 0.0744 | 3639 |
| 3323 | | | | | | | | | | | | | | | | | | |
| Phage- | V | G | P | R | — | V | — | E | — | — | — | I | — | L | 0.0648 | 1.8509 | 0.0954 | 3640 |
| 3324 | | | | | | | | | | | | | | | | | | |
| Phage- | M | Y | I | H | — | V | — | E | — | — | — | R | — | — | 0.0893 | 1.8485 | 0.0941 | 3641 |
| 3325 | | | | | | | | | | | | | | | | | | |
| Phage- | Q | V | — | M | — | T | — | E | — | — | — | S | — | — | 0.0558 | 1.848 | 0.0905 | 3642 |
| 3326 | | | | | | | | | | | | | | | | | | |
| Phage- | — | G | — | V | — | K | — | L | — | — | — | H | — | — | 0.0785 | 1.847 | 0.0971 | 3643 |
| 3327 | | | | | | | | | | | | | | | | | | |
| Phage- | V | — | P | I | — | R | — | S | — | — | — | E | W | L | 0.0723 | 1.8442 | 0.0837 | 3644 |
| 3328 | | | | | | | | | | | | | | | | | | |
| Phage- | V | D | R | — | — | S | — | A | — | — | — | L | — | — | 0.0781 | 1.8441 | 0.0785 | 3645 |
| 3329 | | | | | | | | | | | | | | | | | | |
| Phage- | M | E | A | M | — | R | — | A | — | — | — | — | — | — | 0.0747 | 1.844 | 0.0662 | 3646 |
| 3330 | | | | | | | | | | | | | | | | | | |
| Phage- | V | N | E | — | — | I | — | S | — | — | — | N | — | — | 0.0595 | 1.8438 | 0.0845 | 3647 |
| 3331 | | | | | | | | | | | | | | | | | | |
| Phage- | P | E | L | W | — | V | — | S | — | — | — | E | — | — | 0.068 | 1.8434 | 0.0867 | 3648 |
| 3332 | | | | | | | | | | | | | | | | | | |
| Phage- | — | E | F | — | — | K | — | M | — | — | — | N | — | — | 0.0508 | 1.8426 | 0.0658 | 3649 |
| 3333 | | | | | | | | | | | | | | | | | | |
| Phage- | — | D | R | M | — | K | — | V | — | — | — | E | — | — | 0.061 | 1.8423 | 0.1235 | 3650 |
| 3334 | | | | | | | | | | | | | | | | | | |
| Phage- | E | G | R | Y | — | V | — | E | — | — | — | Q | — | — | 0.0605 | 1.8398 | 0.0864 | 3651 |
| 3335 | | | | | | | | | | | | | | | | | | |
| Phage- | — | V | D | I | — | K | — | S | — | — | — | E | — | — | 0.0627 | 1.8366 | 0.0649 | 3652 |
| 3336 | | | | | | | | | | | | | | | | | | |
| Phage- | L | E | — | — | — | K | — | T | — | — | — | V | — | — | 0.0668 | 1.8358 | 0.078 | 3653 |
| 3337 | | | | | | | | | | | | | | | | | | |
| Phage- | V | D | A | — | — | R | — | S | — | — | — | I | — | — | 0.0772 | 1.8353 | 0.0688 | 3654 |
| 3338 | | | | | | | | | | | | | | | | | | |
| Phage- | T | — | S | M | — | K | — | P | — | — | — | L | — | — | 0.0544 | 1.8344 | 0.0684 | 3655 |
| 3339 | | | | | | | | | | | | | | | | | | |
| Phage- | M | H | F | H | — | K | — | A | — | — | — | Q | — | L | 0.0742 | 1.8341 | 0.0998 | 3656 |
| 3340 | | | | | | | | | | | | | | | | | | |
| Phage- | Q | Y | P | V | — | K | — | A | — | — | — | E | — | — | 0.0674 | 1.8335 | 0.0971 | 3657 |
| 3341 | | | | | | | | | | | | | | | | | | |
| Phage- | M | E | P | V | — | K | — | K | — | — | — | Q | — | — | 0.0661 | 1.8334 | 0.07 | 3658 |
| 3342 | | | | | | | | | | | | | | | | | | |
| Phage- | T | — | P | M | — | K | — | E | — | — | — | V | A | L | 0.075 | 1.8319 | 0.0952 | 3659 |
| 3343 | | | | | | | | | | | | | | | | | | |
| Phage- | M | E | R | — | — | M | — | S | — | — | — | G | — | — | 0.0769 | 1.8311 | 0.0822 | 3660 |
| 3344 | | | | | | | | | | | | | | | | | | |
| Phage- | G | E | L | K | — | I | — | P | — | — | — | E | — | — | 0.0702 | 1.8277 | 0.0808 | 3661 |
| 3345 | | | | | | | | | | | | | | | | | | |
| Phage- | S | E | Y | R | — | V | — | — | — | — | — | S | — | — | 0.0714 | 1.8274 | 0.0791 | 3662 |
| 3346 | | | | | | | | | | | | | | | | | | |
| Phage- | Q | S | P | M | — | K | — | D | — | — | — | L | — | — | 0.0597 | 1.8255 | 0.0728 | 3663 |
| 3347 | | | | | | | | | | | | | | | | | | |
| Phage- | Y | E | Q | R | — | V | — | V | — | — | — | E | — | — | 0.067 | 1.8246 | 0.0675 | 3664 |
| 3348 | | | | | | | | | | | | | | | | | | |
| Phage- | H | I | K | E | — | T | — | R | — | — | — | V | — | — | 0.055 | 1.8236 | 0.078 | 3665 |
| 3349 | | | | | | | | | | | | | | | | | | |
| Phage- | G | D | I | R | — | V | — | E | — | — | — | S | — | L | 0.062 | 1.8221 | 0.0749 | 3666 |
| 3350 | | | | | | | | | | | | | | | | | | |
| Phage- | V | R | P | M | — | E | — | V | — | — | — | M | — | — | 0.0661 | 1.822 | 0.0972 | 3667 |
| 3351 | | | | | | | | | | | | | | | | | | |
| Phage- | V | D | H | V | — | R | — | I | — | — | — | I | — | — | 0.0738 | 1.8216 | 0.0934 | 3668 |
| 3352 | | | | | | | | | | | | | | | | | | |
| Phage- | S | E | H | F | — | R | — | A | — | — | — | T | — | — | 0.059 | 1.8213 | 0.0801 | 3669 |
| 3353 | | | | | | | | | | | | | | | | | | |
| Phage- | G | Q | M | Y | — | K | — | S | — | — | — | N | — | — | 0.1118 | 1.8209 | 0.0788 | 3670 |
| 3354 | | | | | | | | | | | | | | | | | | |
| Phage- | — | G | L | — | — | A | — | K | — | — | — | E | — | — | 0.0854 | 1.82 | 0.0676 | 3671 |
| 3355 | | | | | | | | | | | | | | | | | | |
| Phage- | L | S | W | H | — | I | — | E | — | — | — | — | — | — | 0.0614 | 1.8199 | 0.07 | 3672 |
| 3356 | | | | | | | | | | | | | | | | | | |
| Phage- | D | T | I | Q | — | K | — | A | — | — | — | V | — | — | 0.091 | 1.819 | 0.1025 | 3673 |
| 3357 | | | | | | | | | | | | | | | | | | |
| Phage- | V | I | I | K | — | E | — | R | — | — | — | E | — | — | 0.0543 | 1.819 | 0.068 | 3674 |
| 3358 | | | | | | | | | | | | | | | | | | |
| Phage- | V | E | S | W | — | I | — | A | — | — | — | T | — | — | 0.0628 | 1.8187 | 0.0794 | 3675 |
| 3359 | | | | | | | | | | | | | | | | | | |
| Phage- | F | P | — | R | — | V | — | E | — | — | — | S | — | — | 0.0616 | 1.8179 | 0.0748 | 3676 |
| 3360 | | | | | | | | | | | | | | | | | | |
| Phage- | W | M | P | Y | — | V | — | H | — | — | — | D | — | — | 0.0765 | 1.8178 | 0.1188 | 3677 |
| 3361 | | | | | | | | | | | | | | | | | | |
| Phage- | H | D | L | S | — | V | — | R | — | — | — | D | — | — | 0.0563 | 1.8173 | 0.0671 | 3678 |
| 3362 | | | | | | | | | | | | | | | | | | |
| Phage- | E | D | — | — | — | K | — | L | — | — | — | F | — | — | 0.0645 | 1.8171 | 0.0901 | 3679 |
| 3363 | | | | | | | | | | | | | | | | | | |
| Phage- | E | S | N | F | — | V | — | A | — | — | — | T | — | — | 0.0625 | 1.817 | 0.073 | 3680 |
| 3364 | | | | | | | | | | | | | | | | | | |
| Phage- | F | E | M | — | — | R | — | I | — | — | — | T | — | — | 0.1137 | 1.8166 | 0.1397 | 3681 |
| 3365 | | | | | | | | | | | | | | | | | | |
| Phage- | Q | L | P | M | — | V | — | R | — | — | — | D | — | W | 0.0562 | 1.8163 | 0.1291 | 3682 |
| 3366 | | | | | | | | | | | | | | | | | | |
| Phage- | D | H | — | M | — | K | — | K | — | — | — | E | — | — | 0.0632 | 1.8163 | 0.0947 | 3683 |
| 3367 | | | | | | | | | | | | | | | | | | |
| Phage- | — | — | — | M | — | K | — | E | — | — | — | V | — | — | 0.0677 | 1.816 | 0.0964 | 3684 |
| 3368 | | | | | | | | | | | | | | | | | | |
| Phage- | H | T | P | I | — | V | — | V | — | — | — | — | — | — | 0.0581 | 1.8157 | 0.0746 | 3685 |
| 3369 | | | | | | | | | | | | | | | | | | |
| Phage- | L | S | I | K | — | V | — | D | — | — | — | T | — | — | 0.0668 | 1.8133 | 0.09 | 3686 |
| 3370 | | | | | | | | | | | | | | | | | | |
| Phage- | P | H | A | I | — | K | — | A | — | — | — | V | — | L | 0.082 | 1.8133 | 0.0913 | 3687 |
| 3371 | | | | | | | | | | | | | | | | | | |
| Phage- | D | F | A | M | — | R | — | A | — | — | — | G | — | — | 0.0616 | 1.8123 | 0.0683 | 3688 |
| 3372 | | | | | | | | | | | | | | | | | | |
| Phage- | G | H | P | I | — | V | — | S | — | — | — | E | — | — | 0.0633 | 1.8121 | 0.0862 | 3689 |
| 3373 | | | | | | | | | | | | | | | | | | |
| Phage- | — | D | K | M | — | V | — | S | — | — | — | W | — | — | 0.0624 | 1.8119 | 0.0876 | 3690 |
| 3374 | | | | | | | | | | | | | | | | | | |
| Phage- | Q | E | I | Y | — | V | — | E | — | — | — | S | W | — | 0.0581 | 1.8116 | 0.0642 | 3691 |
| 3375 | | | | | | | | | | | | | | | | | | |
| Phage- | E | Y | I | R | — | V | — | S | — | — | — | L | D | L | 0.0561 | 1.8108 | 0.0615 | 3692 |
| 3376 | | | | | | | | | | | | | | | | | | |
| Phage- | H | D | L | R | — | V | — | E | — | — | — | Y | — | L | 0.0553 | 1.8105 | 0.0734 | 3693 |
| 3377 | | | | | | | | | | | | | | | | | | |
| Phage- | D | V | R | — | — | R | — | S | — | — | — | S | — | — | 0.0707 | 1.81 | 0.0682 | 3694 |
| 3378 | | | | | | | | | | | | | | | | | | |
| Phage- | Q | V | P | M | — | K | — | M | — | — | — | — | — | — | 0.0652 | 1.8099 | 0.0921 | 3695 |
| 3379 | | | | | | | | | | | | | | | | | | |
| Phage- | A | E | — | R | — | V | — | Q | — | — | — | F | — | L | 0.0856 | 1.8089 | 0.0952 | 3696 |
| 3380 | | | | | | | | | | | | | | | | | | |
| Phage- | E | M | R | Y | — | V | — | R | — | — | — | E | — | — | 0.057 | 1.8085 | 0.095 | 3697 |
| 3381 | | | | | | | | | | | | | | | | | | |
| Phage- | V | E | P | — | — | R | — | F | — | — | — | V | — | — | 0.0793 | 1.8068 | 0.1265 | 3698 |
| 3382 | | | | | | | | | | | | | | | | | | |
| Phage- | N | G | A | — | — | K | — | V | — | — | — | Q | — | — | 0.0865 | 1.8059 | 0.0702 | 3699 |
| 3383 | | | | | | | | | | | | | | | | | | |
| Phage- | L | E | — | H | — | R | — | S | — | — | — | V | — | — | 0.0551 | 1.8055 | 0.062 | 3700 |
| 3384 | | | | | | | | | | | | | | | | | | |
| Phage- | — | L | P | Q | — | V | — | S | — | — | — | E | — | — | 0.0815 | 1.8052 | 0.106 | 3701 |
| 3385 | | | | | | | | | | | | | | | | | | |
| Phage- | F | T | A | — | — | K | — | M | — | — | — | R | — | — | 0.0735 | 1.8052 | 0.0718 | 3702 |
| 3386 | | | | | | | | | | | | | | | | | | |
| Phage- | F | S | — | R | — | V | — | E | — | — | — | — | — | — | 0.0651 | 1.8048 | 0.0701 | 3703 |
| 3387 | | | | | | | | | | | | | | | | | | |
| Phage- | M | Q | M | M | — | K | — | D | — | — | — | E | — | — | 0.067 | 1.8046 | 0.0755 | 3704 |
| 3388 | | | | | | | | | | | | | | | | | | |
| Phage- | — | G | A | M | — | E | — | K | — | — | — | F | — | — | 0.0869 | 1.8023 | 0.118 | 3705 |
| 3389 | | | | | | | | | | | | | | | | | | |
| Phage- | Q | G | T | — | — | V | — | S | — | — | — | S | — | — | 0.0514 | 1.8021 | 0.0523 | 3706 |
| 3390 | | | | | | | | | | | | | | | | | | |
| Phage- | — | E | A | V | — | K | — | S | — | — | — | — | — | — | 0.0878 | 1.8017 | 0.0703 | 3707 |
| 3391 | | | | | | | | | | | | | | | | | | |
| Phage- | T | E | I | — | — | V | — | E | — | — | — | I | — | L | 0.0769 | 1.8016 | 0.0705 | 3708 |
| 3392 | | | | | | | | | | | | | | | | | | |
| Phage- | E | N | H | Y | — | R | — | A | — | — | — | N | — | — | 0.0855 | 1.8009 | 0.106 | 3709 |
| 3393 | | | | | | | | | | | | | | | | | | |
| Phage- | — | T | — | I | — | K | — | A | — | — | — | E | F | L | 0.0671 | 1.8009 | 0.0613 | 3710 |
| 3394 | | | | | | | | | | | | | | | | | | |
| Phage- | A | S | — | — | — | V | — | A | — | — | — | S | — | — | 0.0766 | 1.8003 | 0.0808 | 3711 |
| 3395 | | | | | | | | | | | | | | | | | | |
| Phage- | G | F | P | W | — | E | — | Q | — | — | — | I | — | — | 0.0764 | 1.7997 | 0.1137 | 3712 |
| 3396 | | | | | | | | | | | | | | | | | | |
| Phage- | F | G | I | Y | — | V | — | E | — | — | — | T | — | L | 0.0809 | 1.7992 | 0.0891 | 3713 |
| 3397 | | | | | | | | | | | | | | | | | | |
| Phage- | — | E | L | M | — | R | — | L | — | — | — | M | — | — | 0.0679 | 1.7992 | 0.0966 | 3714 |
| 3398 | | | | | | | | | | | | | | | | | | |
| Phage- | T | Q | A | — | — | K | — | A | — | — | — | K | — | — | 0.0556 | 1.7991 | 0.0703 | 3715 |
| 3399 | | | | | | | | | | | | | | | | | | |
| Phage- | F | E | R | — | — | I | — | E | — | — | — | S | A | — | 0.0626 | 1.798 | 0.1015 | 3716 |
| 3400 | | | | | | | | | | | | | | | | | | |
| Phage- | F | R | D | Y | — | V | — | P | — | — | — | V | — | M | 0.0798 | 1.7976 | 0.0791 | 3717 |
| 3401 | | | | | | | | | | | | | | | | | | |
| Phage- | Q | F | L | V | — | K | — | S | — | — | — | N | — | — | 0.0584 | 1.7971 | 0.0882 | 3718 |
| 3402 | | | | | | | | | | | | | | | | | | |
| Phage- | V | E | R | Y | — | V | — | Q | — | — | — | D | — | — | 0.0596 | 1.7966 | 0.1175 | 3719 |
| 3403 | | | | | | | | | | | | | | | | | | |
| Phage- | A | — | L | R | — | V | — | T | — | — | — | E | — | — | 0.0707 | 1.7949 | 0.0793 | 3720 |
| 3404 | | | | | | | | | | | | | | | | | | |
| Phage- | Q | L | — | — | — | V | — | E | — | — | — | L | — | L | 0.0949 | 1.7945 | 0.0659 | 3721 |
| 3405 | | | | | | | | | | | | | | | | | | |
| Phage- | A | Q | G | V | — | I | — | Q | — | — | — | D | — | — | 0.0574 | 1.7944 | 0.0715 | 3722 |
| 3406 | | | | | | | | | | | | | | | | | | |
| Phage- | S | E | I | — | — | R | — | A | — | — | — | T | — | — | 0.0591 | 1.7943 | 0.1037 | 3723 |
| 3407 | | | | | | | | | | | | | | | | | | |
| Phage- | G | D | H | Y | — | K | — | D | — | — | — | S | — | — | 0.0602 | 1.7942 | 0.071 | 3724 |
| 3408 | | | | | | | | | | | | | | | | | | |
| Phage- | D | T | F | M | — | R | — | S | — | — | — | N | — | — | 0.0651 | 1.7939 | 0.072 | 3725 |
| 3409 | | | | | | | | | | | | | | | | | | |
| Phage- | V | E | P | — | — | R | — | M | — | — | — | E | — | I | 0.0866 | 1.7915 | 0.1222 | 3726 |
| 3410 | | | | | | | | | | | | | | | | | | |
| Phage- | S | L | A | Q | — | V | — | E | — | — | — | F | — | — | 0.0658 | 1.7902 | 0.1134 | 3727 |
| 3411 | | | | | | | | | | | | | | | | | | |
| Phage- | E | L | — | R | — | I | — | V | — | — | — | Q | — | — | 0.0799 | 1.7895 | 0.3685 | 3728 |
| 3412 | | | | | | | | | | | | | | | | | | |
| Phage- | S | V | K | V | — | K | — | A | — | — | — | — | — | — | 0.0669 | 1.7886 | 0.102 | 3729 |
| 3413 | | | | | | | | | | | | | | | | | | |
| Phage- | D | F | — | R | — | V | — | D | — | — | — | Q | — | — | 0.0588 | 1.7859 | 0.0627 | 3730 |
| 3414 | | | | | | | | | | | | | | | | | | |
| Phage- | M | E | T | W | — | V | — | T | — | — | — | T | — | — | 0.0582 | 1.7856 | 0.0748 | 3731 |
| 3415 | | | | | | | | | | | | | | | | | | |
| Phage- | D | T | W | I | — | K | — | E | — | — | — | M | — | — | 0.1032 | 1.7842 | 0.0802 | 3732 |
| 3416 | | | | | | | | | | | | | | | | | | |
| Phage- | V | E | R | A | — | R | — | V | — | — | — | Q | — | — | 0.0771 | 1.7839 | 0.0907 | 3733 |
| 3417 | | | | | | | | | | | | | | | | | | |
| Phage- | — | M | P | T | — | V | — | Q | — | — | — | N | — | — | 0.0604 | 1.7823 | 0.093 | 3734 |
| 3418 | | | | | | | | | | | | | | | | | | |
| Phage- | M | L | P | Y | — | V | — | Q | — | — | — | M | — | — | 0.0783 | 1.7815 | 0.147 | 3735 |
| 3419 | | | | | | | | | | | | | | | | | | |
| Phage- | N | D | — | K | — | I | — | E | — | — | — | V | — | — | 0.0712 | 1.7812 | 0.0849 | 3736 |
| 3420 | | | | | | | | | | | | | | | | | | |
| Phage- | T | D | P | — | — | I | — | E | — | — | — | Q | — | M | 0.0602 | 1.7812 | 0.0726 | 3737 |
| 3421 | | | | | | | | | | | | | | | | | | |
| Phage- | L | S | T | — | — | I | — | Q | — | — | — | T | — | L | 0.0596 | 1.781 | 0.0813 | 3738 |
| 3422 | | | | | | | | | | | | | | | | | | |
| Phage- | Y | F | Q | M | — | A | — | R | — | — | — | E | — | — | 0.0994 | 1.7784 | 0.0849 | 3739 |
| 3423 | | | | | | | | | | | | | | | | | | |
| Phage- | — | D | Q | I | — | K | — | A | — | — | — | S | — | — | 0.0553 | 1.7776 | 0.0628 | 3740 |
| 3424 | | | | | | | | | | | | | | | | | | |
| Phage- | L | E | I | M | — | R | — | E | — | — | — | S | — | — | 0.076 | 1.7724 | 0.0888 | 3741 |
| 3425 | | | | | | | | | | | | | | | | | | |
| Phage- | M | T | P | R | — | I | — | S | — | — | — | F | — | M | 0.0816 | 1.7711 | 0.086 | 3742 |
| 3426 | | | | | | | | | | | | | | | | | | |
| Phage- | S | S | M | — | — | I | — | T | — | — | — | G | — | — | 0.0545 | 1.7705 | 0.0536 | 3743 |
| 3427 | | | | | | | | | | | | | | | | | | |
| Phage- | T | S | L | R | — | V | — | E | — | — | — | S | — | — | 0.0569 | 1.7704 | 0.0814 | 3744 |
| 3428 | | | | | | | | | | | | | | | | | | |
| Phage- | S | L | P | I | — | K | — | E | — | — | — | V | — | L | 0.0666 | 1.767 | 0.0892 | 3745 |
| 3429 | | | | | | | | | | | | | | | | | | |
| Phage- | V | E | K | M | — | K | — | V | — | — | — | N | — | — | 0.0796 | 1.7666 | 0.1148 | 3746 |
| 3430 | | | | | | | | | | | | | | | | | | |
| Phage- | E | D | I | — | — | K | — | V | — | — | — | I | — | — | 0.0563 | 1.7659 | 0.06 | 3747 |
| 3431 | | | | | | | | | | | | | | | | | | |
| Phage- | S | F | L | R | — | E | — | M | — | — | — | L | — | — | 0.0766 | 1.7651 | 0.0816 | 3748 |
| 3432 | | | | | | | | | | | | | | | | | | |
| Phage- | — | E | P | H | — | V | — | A | — | — | — | T | — | — | 0.0583 | 1.7629 | 0.0805 | 3749 |
| 3433 | | | | | | | | | | | | | | | | | | |
| Phage- | E | I | P | Y | — | V | — | Q | — | — | — | V | A | M | 0.086 | 1.7623 | 0.0788 | 3750 |
| 3434 | | | | | | | | | | | | | | | | | | |
| Phage- | V | L | H | M | — | V | — | L | — | — | — | H | — | L | 0.0809 | 1.7619 | 0.1326 | 3751 |
| 3435 | | | | | | | | | | | | | | | | | | |
| Phage- | V | K | P | V | — | K | — | V | — | — | — | V | — | — | 0.0829 | 1.7616 | 0.1217 | 3752 |
| 3436 | | | | | | | | | | | | | | | | | | |
| Phage- | P | G | P | S | — | V | — | S | — | — | — | V | — | — | 0.0518 | 1.7593 | 0.074 | 3753 |
| 3437 | | | | | | | | | | | | | | | | | | |
| Phage- | P | — | Q | M | — | I | — | Q | — | — | — | N | W | L | 0.0562 | 1.7544 | 0.0635 | 3754 |
| 3438 | | | | | | | | | | | | | | | | | | |
| Phage- | S | P | I | M | — | T | — | D | — | — | — | L | — | — | 0.0552 | 1.7541 | 0.0708 | 3755 |
| 3439 | | | | | | | | | | | | | | | | | | |
| Phage- | P | V | R | V | — | I | — | Q | — | — | — | I | — | — | 0.0776 | 1.7536 | 0.1301 | 3756 |
| 3440 | | | | | | | | | | | | | | | | | | |
| Phage- | E | T | M | I | — | K | — | E | — | — | — | T | — | — | 0.0557 | 1.7526 | 0.0818 | 3757 |
| 3441 | | | | | | | | | | | | | | | | | | |
| Phage- | V | T | P | R | — | I | — | E | — | — | — | — | — | — | 0.0769 | 1.7517 | 0.1062 | 3758 |
| 3442 | | | | | | | | | | | | | | | | | | |
| Phage- | Y | G | L | R | — | E | — | A | — | — | — | Q | — | — | 0.102 | 1.7507 | 0.0879 | 3759 |
| 3443 | | | | | | | | | | | | | | | | | | |
| Phage- | T | P | H | A | — | V | — | D | — | — | — | Y | — | — | 0.0602 | 1.7506 | 0.0843 | 3760 |
| 3444 | | | | | | | | | | | | | | | | | | |
| Phage- | — | T | P | M | — | I | — | A | — | — | — | E | — | V | 0.0534 | 1.7502 | 0.0611 | 3761 |
| 3445 | | | | | | | | | | | | | | | | | | |
| Phage- | M | P | I | Y | — | V | — | E | — | — | — | Q | — | L | 0.0757 | 1.75 | 0.0867 | 3762 |
| 3446 | | | | | | | | | | | | | | | | | | |
| Phage- | Y | N | M | I | — | — | — | Q | — | — | — | S | — | — | 0.0598 | 1.7487 | 0.0661 | 3763 |
| 3447 | | | | | | | | | | | | | | | | | | |
| Phage- | L | E | K | V | — | K | — | M | — | — | — | T | — | — | 0.0624 | 1.7487 | 0.0949 | 3764 |
| 3448 | | | | | | | | | | | | | | | | | | |
| Phage- | E | D | — | R | — | V | — | A | — | — | — | N | — | — | 0.0625 | 1.7475 | 0.0724 | 3765 |
| 3449 | | | | | | | | | | | | | | | | | | |
| Phage- | P | L | K | — | — | I | — | E | — | — | — | V | E | L | 0.0636 | 1.7463 | 0.0747 | 3766 |
| 3450 | | | | | | | | | | | | | | | | | | |
| Phage- | T | I | N | I | — | K | — | Q | — | — | — | I | — | — | 0.0849 | 1.7449 | 0.0893 | 3767 |
| 3451 | | | | | | | | | | | | | | | | | | |
| Phage- | M | E | R | F | — | I | — | S | — | — | — | E | — | L | 0.0578 | 1.7444 | 0.0682 | 3768 |
| 3452 | | | | | | | | | | | | | | | | | | |
| Phage- | F | E | F | R | — | T | — | E | — | — | — | R | — | — | 0.0795 | 1.7435 | 0.0706 | 3769 |
| 3453 | | | | | | | | | | | | | | | | | | |
| Phage- | V | T | M | R | — | T | — | D | — | — | — | V | — | — | 0.0674 | 1.7425 | 0.0737 | 3770 |
| 3454 | | | | | | | | | | | | | | | | | | |
| Phage- | — | E | A | — | — | V | — | D | — | — | — | R | — | — | 0.0583 | 1.7425 | 0.0663 | 3771 |
| 3455 | | | | | | | | | | | | | | | | | | |
| Phage- | H | I | M | Q | — | K | — | A | — | — | — | S | — | L | 0.0633 | 1.7422 | 0.0654 | 3772 |
| 3456 | | | | | | | | | | | | | | | | | | |
| Phage- | V | E | K | M | — | R | — | L | — | — | — | H | — | — | 0.0706 | 1.7418 | 0.0944 | 3773 |
| 3457 | | | | | | | | | | | | | | | | | | |
| Phage- | V | V | A | Q | — | V | — | R | — | — | — | D | — | — | 0.093 | 1.7417 | 0.1035 | 3774 |
| 3458 | | | | | | | | | | | | | | | | | | |
| Phage- | S | P | P | — | — | V | — | V | — | — | — | H | — | — | 0.0835 | 1.7413 | 0.1478 | 3775 |
| 3459 | | | | | | | | | | | | | | | | | | |
| Phage- | N | — | I | M | — | R | — | A | — | — | — | S | — | — | 0.0757 | 1.7411 | 0.0847 | 3776 |
| 3460 | | | | | | | | | | | | | | | | | | |
| Phage- | T | E | L | — | — | T | — | R | — | — | — | E | — | — | 0.1123 | 1.7403 | 0.1629 | 3777 |
| 3461 | | | | | | | | | | | | | | | | | | |
| Phage- | V | L | R | S | — | V | — | E | — | — | — | S | — | — | 0.0651 | 1.7403 | 0.075 | 3778 |
| 3462 | | | | | | | | | | | | | | | | | | |
| Phage- | E | V | L | — | — | K | — | E | — | — | — | T | — | — | 0.0724 | 1.7393 | 0.0669 | 3779 |
| 3463 | | | | | | | | | | | | | | | | | | |
| Phage- | E | I | K | H | — | I | — | E | — | — | — | S | — | — | 0.0712 | 1.7385 | 0.1045 | 3780 |
| 3464 | | | | | | | | | | | | | | | | | | |
| Phage- | E | T | H | F | — | K | — | K | — | — | — | V | — | — | 0.0657 | 1.738 | 0.0927 | 3781 |
| 3465 | | | | | | | | | | | | | | | | | | |
| Phage- | L | E | A | A | — | R | — | M | — | — | — | L | — | — | 0.0603 | 1.7341 | 0.0664 | 3782 |
| 3466 | | | | | | | | | | | | | | | | | | |
| Phage- | W | E | A | R | — | — | — | T | — | — | — | T | — | — | 0.0583 | 1.7336 | 0.0667 | 3783 |
| 3467 | | | | | | | | | | | | | | | | | | |
| Phage- | T | N | P | M | — | V | — | E | — | — | — | Y | — | — | 0.0527 | 1.7319 | 0.0628 | 3784 |
| 3468 | | | | | | | | | | | | | | | | | | |
| Phage- | E | I | — | K | — | V | — | P | — | — | — | S | — | I | 0.0735 | 1.73 | 0.0727 | 3785 |
| 3469 | | | | | | | | | | | | | | | | | | |
| Phage- | V | D | Q | V | — | I | — | D | — | — | — | N | — | — | 0.1695 | 1.7293 | 0.0771 | 3786 |
| 3470 | | | | | | | | | | | | | | | | | | |
| Phage- | S | V | P | S | — | V | — | P | — | — | — | I | — | — | 0.0657 | 1.7291 | 0.0613 | 3787 |
| 3471 | | | | | | | | | | | | | | | | | | |
| Phage- | Q | E | — | W | — | R | — | I | — | — | — | E | — | — | 0.0867 | 1.7285 | 0.1086 | 3788 |
| 3472 | | | | | | | | | | | | | | | | | | |
| Phage- | E | G | T | W | — | R | — | A | — | — | — | D | — | — | 0.0539 | 1.7276 | 0.0819 | 3789 |
| 3473 | | | | | | | | | | | | | | | | | | |
| Phage- | L | E | R | A | — | K | — | M | — | — | — | E | — | — | 0.058 | 1.7275 | 0.0863 | 3790 |
| 3474 | | | | | | | | | | | | | | | | | | |
| Phage- | E | D | R | I | — | E | — | D | — | — | — | — | — | — | 0.0562 | 1.7269 | 0.1051 | 3791 |
| 3475 | | | | | | | | | | | | | | | | | | |
| Phage- | V | S | P | M | — | R | — | A | — | — | — | E | — | L | 0.0563 | 1.7268 | 0.0909 | 3792 |
| 3476 | | | | | | | | | | | | | | | | | | |
| Phage- | V | W | K | T | — | V | — | E | — | — | — | V | — | Y | 0.0753 | 1.7268 | 0.1119 | 3793 |
| 3477 | | | | | | | | | | | | | | | | | | |
| Phage- | S | D | K | F | — | V | — | E | — | — | — | E | W | I | 0.0517 | 1.7246 | 0.0588 | 3794 |
| 3478 | | | | | | | | | | | | | | | | | | |
| Phage- | M | D | A | R | — | V | — | S | — | — | — | I | — | V | 0.0708 | 1.7245 | 0.48 | 3795 |
| 3479 | | | | | | | | | | | | | | | | | | |
| Phage- | E | F | I | K | — | E | — | K | — | — | — | Q | — | — | 0.0686 | 1.7223 | 0.0826 | 3796 |
| 3480 | | | | | | | | | | | | | | | | | | |
| Phage- | D | T | I | A | — | R | — | E | — | — | — | T | — | — | 0.0524 | 1.7217 | 0.0607 | 3797 |
| 3481 | | | | | | | | | | | | | | | | | | |
| Phage- | Y | F | K | H | — | V | — | Q | — | — | — | E | — | — | 0.0663 | 1.7207 | 0.1464 | 3798 |
| 3482 | | | | | | | | | | | | | | | | | | |
| Phage- | — | T | P | A | — | V | — | E | — | — | — | L | — | L | 0.0587 | 1.7192 | 0.0774 | 3799 |
| 3483 | | | | | | | | | | | | | | | | | | |
| Phage- | A | E | R | M | — | V | — | S | — | — | — | Q | — | L | 0.0621 | 1.7192 | 0.0943 | 3800 |
| 3484 | | | | | | | | | | | | | | | | | | |
| Phage- | E | — | I | R | — | V | — | V | — | — | — | Q | — | — | 0.0761 | 1.718 | 0.0989 | 3801 |
| 3485 | | | | | | | | | | | | | | | | | | |
| Phage- | Y | S | Q | M | — | K | — | A | — | — | — | N | — | — | 0.0744 | 1.7173 | 0.1012 | 3802 |
| 3486 | | | | | | | | | | | | | | | | | | |
| Phage- | P | V | A | V | — | K | — | A | — | — | — | E | — | — | 0.0554 | 1.716 | 0.0982 | 3803 |
| 3487 | | | | | | | | | | | | | | | | | | |
| Phage- | — | T | Q | M | — | K | — | A | — | — | — | V | — | L | 0.0811 | 1.7155 | 0.1229 | 3804 |
| 3488 | | | | | | | | | | | | | | | | | | |
| Phage- | L | D | R | M | — | V | — | L | — | — | — | E | — | — | 0.0777 | 1.714 | 0.0991 | 3805 |
| 3489 | | | | | | | | | | | | | | | | | | |
| Phage- | V | N | M | I | — | K | — | A | — | — | — | K | — | — | 0.0606 | 1.7128 | 0.0872 | 3806 |
| 3490 | | | | | | | | | | | | | | | | | | |
| Phage- | L | E | M | V | — | K | — | E | — | — | — | L | — | L | 0.051 | 1.7082 | 0.0996 | 3807 |
| 3491 | | | | | | | | | | | | | | | | | | |
| Phage- | D | I | L | R | — | V | — | E | — | — | — | T | — | — | 0.0796 | 1.7074 | 0.0911 | 3808 |
| 3492 | | | | | | | | | | | | | | | | | | |
| Phage- | Y | P | T | W | — | V | — | T | — | — | — | T | — | M | 0.0592 | 1.7062 | 0.0716 | 3809 |
| 3493 | | | | | | | | | | | | | | | | | | |
| Phage- | D | Y | M | M | — | S | — | D | — | — | — | K | — | — | 0.0607 | 1.703 | 0.0722 | 3810 |
| 3494 | | | | | | | | | | | | | | | | | | |
| Phage- | S | M | A | W | — | V | — | — | — | — | — | T | — | — | 0.0884 | 1.7021 | 0.0897 | 3811 |
| 3495 | | | | | | | | | | | | | | | | | | |
| Phage- | Q | D | M | V | — | R | — | A | — | — | — | T | — | — | 0.062 | 1.7014 | 0.0848 | 3812 |
| 3496 | | | | | | | | | | | | | | | | | | |
| Phage- | V | D | R | I | — | K | — | D | — | — | — | — | — | — | 0.0654 | 1.7001 | 0.091 | 3813 |
| 3497 | | | | | | | | | | | | | | | | | | |
| Phage- | T | V | H | Y | — | T | — | S | — | — | — | T | — | — | 0.0803 | 1.6998 | 0.0866 | 3814 |
| 3498 | | | | | | | | | | | | | | | | | | |
| Phage- | T | H | P | — | — | V | — | T | — | — | — | S | — | — | 0.0807 | 1.6992 | 0.0862 | 3815 |
| 3499 | | | | | | | | | | | | | | | | | | |
| Phage- | Y | D | H | F | — | V | — | R | — | — | — | E | — | L | 0.0573 | 1.6989 | 0.0592 | 3816 |
| 3500 | | | | | | | | | | | | | | | | | | |
| Phage- | P | L | R | Y | — | E | — | A | — | — | — | F | — | — | 0.0534 | 1.6986 | 0.0601 | 3817 |
| 3501 | | | | | | | | | | | | | | | | | | |
| Phage- | W | L | Y | S | — | I | — | Q | — | — | — | T | — | — | 0.0938 | 1.6973 | 0.1273 | 3818 |
| 3502 | | | | | | | | | | | | | | | | | | |
| Phage- | E | — | P | A | — | V | — | V | — | — | — | N | — | — | 0.0543 | 1.6969 | 0.0778 | 3819 |
| 3503 | | | | | | | | | | | | | | | | | | |
| Phage- | N | I | I | H | — | V | — | P | — | — | — | D | — | — | 0.0625 | 1.6967 | 0.0795 | 3820 |
| 3504 | | | | | | | | | | | | | | | | | | |
| Phage- | — | E | I | R | — | K | — | E | — | — | — | T | S | — | 0.0858 | 1.6965 | 0.0708 | 3821 |
| 3505 | | | | | | | | | | | | | | | | | | |
| Phage- | L | T | Q | M | — | K | — | L | — | — | — | — | — | — | 0.0798 | 1.694 | 0.0786 | 3822 |
| 3506 | | | | | | | | | | | | | | | | | | |
| Phage- | A | E | P | V | — | V | — | D | — | — | — | F | — | — | 0.065 | 1.6936 | 0.1035 | 3823 |
| 3507 | | | | | | | | | | | | | | | | | | |
| Phage- | V | D | H | R | — | V | — | S | — | — | — | Y | — | L | 0.0722 | 1.6932 | 0.0745 | 3824 |
| 3508 | | | | | | | | | | | | | | | | | | |
| Phage- | E | Y | M | R | — | E | — | S | — | — | — | Y | — | — | 0.0532 | 1.6926 | 0.0584 | 3825 |
| 3509 | | | | | | | | | | | | | | | | | | |
| Phage- | A | D | I | Y | — | K | — | V | — | — | — | T | — | — | 0.0741 | 1.6914 | 0.0909 | 3826 |
| 3510 | | | | | | | | | | | | | | | | | | |
| Phage- | V | G | H | M | — | K | — | V | — | — | — | — | — | L | 0.0823 | 1.6902 | 0.101 | 3827 |
| 3511 | | | | | | | | | | | | | | | | | | |
| Phage- | T | E | Q | I | — | K | — | A | — | — | — | T | — | L | 0.053 | 1.69 | 0.0536 | 3828 |
| 3512 | | | | | | | | | | | | | | | | | | |
| Phage- | S | V | T | T | — | I | — | P | — | — | — | T | — | — | 0.0658 | 1.6895 | 0.0979 | 3829 |
| 3513 | | | | | | | | | | | | | | | | | | |
| Phage- | V | V | K | H | — | V | — | T | — | — | — | — | — | — | 0.0933 | 1.6877 | 0.0813 | 3830 |
| 3514 | | | | | | | | | | | | | | | | | | |
| Phage- | P | E | L | A | — | I | — | E | — | — | — | I | — | — | 0.053 | 1.6869 | 0.0803 | 3831 |
| 3515 | | | | | | | | | | | | | | | | | | |
| Phage- | S | V | A | Y | — | V | — | T | — | — | — | D | — | L | 0.0603 | 1.6861 | 0.0618 | 3832 |
| 3516 | | | | | | | | | | | | | | | | | | |
| Phage- | M | V | P | R | — | E | — | E | — | — | — | V | — | — | 0.0709 | 1.6854 | 0.0705 | 3833 |
| 3517 | | | | | | | | | | | | | | | | | | |
| Phage- | G | P | K | I | — | V | — | M | — | — | — | D | — | — | 0.0833 | 1.6848 | 0.1164 | 3834 |
| 3518 | | | | | | | | | | | | | | | | | | |
| Phage- | S | — | I | R | — | E | — | L | — | — | — | S | — | L | 0.0561 | 1.6844 | 0.0578 | 3835 |
| 3519 | | | | | | | | | | | | | | | | | | |
| Phage- | E | V | Q | Y | — | V | — | A | — | — | — | E | — | — | 0.0832 | 1.683 | 0.1167 | 3836 |
| 3520 | | | | | | | | | | | | | | | | | | |
| Phage- | P | S | I | S | — | T | — | Q | — | — | — | F | — | — | 0.0672 | 1.6814 | 0.0639 | 3837 |
| 3521 | | | | | | | | | | | | | | | | | | |
| Phage- | A | L | L | S | — | V | — | P | — | — | — | L | — | L | 0.0846 | 1.6792 | 0.0728 | 3838 |
| 3522 | | | | | | | | | | | | | | | | | | |
| Phage- | D | M | A | R | — | I | — | A | — | — | — | T | — | — | 0.0883 | 1.6754 | 0.1062 | 3839 |
| 3523 | | | | | | | | | | | | | | | | | | |
| Phage- | E | L | L | H | — | K | — | M | — | — | — | I | — | L | 0.0686 | 1.6726 | 0.0864 | 3840 |
| 3524 | | | | | | | | | | | | | | | | | | |
| Phage- | Y | P | P | — | — | K | — | E | — | — | — | V | — | L | 0.0544 | 1.6706 | 0.0714 | 3841 |
| 3525 | | | | | | | | | | | | | | | | | | |
| Phage- | L | I | A | — | — | K | — | P | — | — | — | E | W | — | 0.0985 | 1.67 | 0.2133 | 3842 |
| 3526 | | | | | | | | | | | | | | | | | | |
| Phage- | W | F | R | M | — | E | — | S | — | — | — | E | — | — | 0.0639 | 1.6694 | 0.1099 | 3843 |
| 3527 | | | | | | | | | | | | | | | | | | |
| Phage- | G | Y | — | M | — | E | — | R | — | — | — | E | — | L | 0.0867 | 1.6672 | 0.0803 | 3844 |
| 3528 | | | | | | | | | | | | | | | | | | |
| Phage- | V | V | P | W | — | S | — | A | — | — | — | S | — | — | 0.068 | 1.6665 | 0.0752 | 3845 |
| 3529 | | | | | | | | | | | | | | | | | | |
| Phage- | E | E | R | I | — | K | — | Q | — | — | — | S | A | — | 0.0549 | 1.6643 | 0.0804 | 3846 |
| 3530 | | | | | | | | | | | | | | | | | | |
| Phage- | V | S | P | S | — | V | — | P | — | — | — | V | — | — | 0.0628 | 1.6641 | 0.0791 | 3847 |
| 3531 | | | | | | | | | | | | | | | | | | |
| Phage- | V | V | R | H | — | V | — | T | — | — | — | T | — | — | 0.07 | 1.6628 | 0.1022 | 3848 |
| 3532 | | | | | | | | | | | | | | | | | | |
| Phage- | V | E | R | V | — | V | — | Q | — | — | — | S | — | — | 0.0614 | 1.6623 | 0.1003 | 3849 |
| 3533 | | | | | | | | | | | | | | | | | | |
| Phage- | E | S | — | Y | — | I | — | Q | — | — | — | S | — | L | 0.0717 | 1.6606 | 0.0698 | 3850 |
| 3534 | | | | | | | | | | | | | | | | | | |
| Phage- | L | N | A | M | — | V | — | A | — | — | — | T | — | — | 0.0559 | 1.6582 | 0.0634 | 3851 |
| 3535 | | | | | | | | | | | | | | | | | | |
| Phage- | L | E | M | A | — | R | — | M | — | — | — | Q | — | L | 0.0741 | 1.6581 | 0.0725 | 3852 |
| 3536 | | | | | | | | | | | | | | | | | | |
| Phage- | F | E | — | A | — | I | — | Q | — | — | — | D | — | — | 0.0701 | 1.6579 | 0.1181 | 3853 |
| 3537 | | | | | | | | | | | | | | | | | | |
| Phage- | — | — | R | T | — | V | — | A | — | — | — | S | — | — | 0.0701 | 1.6549 | 0.1022 | 3854 |
| 3538 | | | | | | | | | | | | | | | | | | |
| Phage- | D | V | R | M | — | V | — | E | — | — | — | S | — | — | 0.057 | 1.6542 | 0.0753 | 3855 |
| 3539 | | | | | | | | | | | | | | | | | | |
| Phage- | V | K | P | M | — | V | — | E | — | — | — | S | — | L | 0.071 | 1.6535 | 0.0907 | 3856 |
| 3540 | | | | | | | | | | | | | | | | | | |
| Phage- | E | N | L | I | — | K | — | A | — | — | — | V | — | — | 0.0689 | 1.652 | 0.0738 | 3857 |
| 3541 | | | | | | | | | | | | | | | | | | |
| Phage- | L | Y | — | K | — | S | — | E | — | — | — | I | — | — | 0.0737 | 1.65 | 0.0709 | 3858 |
| 3542 | | | | | | | | | | | | | | | | | | |
| Phage- | V | T | P | Q | — | V | — | M | — | — | — | T | — | — | 0.0773 | 1.6499 | 0.1047 | 3859 |
| 3543 | | | | | | | | | | | | | | | | | | |
| Phage- | F | L | Q | A | — | K | — | P | — | — | — | T | — | — | 0.1128 | 1.6485 | 0.1461 | 3860 |
| 3544 | | | | | | | | | | | | | | | | | | |
| Phage- | V | V | K | A | — | I | — | E | — | — | — | S | — | — | 0.0631 | 1.6483 | 0.1252 | 3861 |
| 3545 | | | | | | | | | | | | | | | | | | |
| Phage- | D | — | R | A | — | V | — | E | — | — | — | Y | — | — | 0.0633 | 1.6478 | 0.0805 | 3862 |
| 3546 | | | | | | | | | | | | | | | | | | |
| Phage- | T | E | R | V | — | V | — | D | — | — | — | — | — | — | 0.0569 | 1.6473 | 0.0944 | 3863 |
| 3547 | | | | | | | | | | | | | | | | | | |
| Phage- | T | P | P | F | — | I | — | K | — | — | — | Q | — | — | 0.0744 | 1.6469 | 0.1172 | 3864 |
| 3548 | | | | | | | | | | | | | | | | | | |
| Phage- | M | N | I | M | — | R | — | V | — | — | — | T | — | L | 0.0855 | 1.6467 | 0.0993 | 3865 |
| 3549 | | | | | | | | | | | | | | | | | | |
| Phage- | M | E | R | M | — | V | — | S | — | — | — | T | — | — | 0.0612 | 1.6448 | 0.089 | 3866 |
| 3550 | | | | | | | | | | | | | | | | | | |
| Phage- | — | E | K | V | — | V | — | A | — | — | — | M | — | — | 0.068 | 1.6437 | 0.2382 | 3867 |
| 3551 | | | | | | | | | | | | | | | | | | |
| Phage- | V | D | R | — | — | V | — | — | — | — | — | M | — | L | 0.1184 | 1.6434 | 0.072 | 3868 |
| 3552 | | | | | | | | | | | | | | | | | | |
| Phage- | D | P | E | K | — | M | — | A | — | — | — | S | — | — | 0.1069 | 1.642 | 0.0775 | 3869 |
| 3553 | | | | | | | | | | | | | | | | | | |
| Phage- | A | G | M | M | — | T | — | P | — | — | — | F | — | — | 0.0716 | 1.6414 | 0.0664 | 3870 |
| 3554 | | | | | | | | | | | | | | | | | | |
| Phage- | L | — | — | R | — | I | — | E | — | — | — | Y | — | L | 0.0618 | 1.6412 | 0.0644 | 3871 |
| 3555 | | | | | | | | | | | | | | | | | | |
| Phage- | Q | S | M | M | — | I | — | S | — | — | — | V | — | — | 0.0578 | 1.6405 | 0.0679 | 3872 |
| 3556 | | | | | | | | | | | | | | | | | | |
| Phage- | V | V | P | — | — | V | — | M | — | — | — | S | P | M | 0.0824 | 1.6389 | 0.0867 | 3873 |
| 3557 | | | | | | | | | | | | | | | | | | |
| Phage- | V | E | P | A | — | V | — | L | — | — | — | F | — | L | 0.0539 | 1.6388 | 0.0619 | 3874 |
| 3558 | | | | | | | | | | | | | | | | | | |
| Phage- | T | Y | F | R | — | E | — | S | — | — | — | T | — | L | 0.0635 | 1.6375 | 0.073 | 3875 |
| 3559 | | | | | | | | | | | | | | | | | | |
| Phage- | M | E | R | W | — | I | — | V | — | — | — | Q | — | L | 0.0596 | 1.6374 | 0.0734 | 3876 |
| 3560 | | | | | | | | | | | | | | | | | | |
| Phage- | V | V | N | F | — | K | — | Q | — | — | — | S | — | — | 0.0815 | 1.6372 | 0.0682 | 3877 |
| 3561 | | | | | | | | | | | | | | | | | | |
| Phage- | D | D | I | R | — | E | — | I | — | — | — | T | — | — | 0.0528 | 1.6368 | 0.0657 | 3878 |
| 3562 | | | | | | | | | | | | | | | | | | |
| Phage- | E | H | R | I | — | V | — | Q | — | — | — | T | — | — | 0.0513 | 1.6365 | 0.0676 | 3879 |
| 3563 | | | | | | | | | | | | | | | | | | |
| Phage- | S | P | S | Y | — | V | — | K | — | — | — | V | — | — | 0.055 | 1.6351 | 0.0712 | 3880 |
| 3564 | | | | | | | | | | | | | | | | | | |
| Phage- | E | L | L | K | — | V | — | Q | — | — | — | T | — | — | 0.069 | 1.6314 | 0.1264 | 3881 |
| 3565 | | | | | | | | | | | | | | | | | | |
| Phage- | V | S | T | M | — | V | — | E | — | — | — | G | — | — | 0.0592 | 1.6307 | 0.1218 | 3882 |
| 3566 | | | | | | | | | | | | | | | | | | |
| Phage- | D | — | M | — | — | R | — | S | — | — | — | D | — | — | 0.0658 | 1.6243 | 0.0871 | 3883 |
| 3567 | | | | | | | | | | | | | | | | | | |
| Phage- | P | E | A | Y | — | V | — | S | — | — | — | T | — | — | 0.0819 | 1.6241 | 0.0764 | 3884 |
| 3568 | | | | | | | | | | | | | | | | | | |
| Phage- | V | — | Q | W | — | T | — | S | — | — | — | D | — | — | 0.0693 | 1.6236 | 0.0794 | 3885 |
| 3569 | | | | | | | | | | | | | | | | | | |
| Phage- | V | D | K | A | — | T | — | A | — | — | — | T | — | — | 0.0659 | 1.6232 | 0.0716 | 3886 |
| 3570 | | | | | | | | | | | | | | | | | | |
| Phage- | E | M | P | — | — | I | — | Q | — | — | — | G | — | — | 0.0677 | 1.6218 | 0.0691 | 3887 |
| 3571 | | | | | | | | | | | | | | | | | | |
| Phage- | V | P | T | — | — | K | — | S | — | — | — | Y | — | — | 0.0774 | 1.6217 | 0.0941 | 3888 |
| 3572 | | | | | | | | | | | | | | | | | | |
| Phage- | A | W | M | R | — | E | — | A | — | — | — | V | — | — | 0.0559 | 1.621 | 0.0695 | 3889 |
| 3573 | | | | | | | | | | | | | | | | | | |
| Phage- | E | D | I | K | — | I | — | R | — | — | — | E | — | — | 0.0691 | 1.621 | 0.0923 | 3890 |
| 3574 | | | | | | | | | | | | | | | | | | |
| Phage- | F | I | A | Q | — | — | — | T | — | — | — | T | — | — | 0.0654 | 1.6204 | 0.076 | 3891 |
| 3575 | | | | | | | | | | | | | | | | | | |
| Phage- | E | T | F | T | — | K | — | P | — | — | — | I | — | — | 0.0767 | 1.6202 | 0.1045 | 3892 |
| 3576 | | | | | | | | | | | | | | | | | | |
| Phage- | Y | E | K | — | — | E | — | R | — | — | — | D | — | — | 0.0525 | 1.6197 | 0.0657 | 3893 |
| 3577 | | | | | | | | | | | | | | | | | | |
| Phage- | Y | D | — | V | — | K | — | A | — | — | — | N | — | — | 0.0572 | 1.619 | 0.0676 | 3894 |
| 3578 | | | | | | | | | | | | | | | | | | |
| Phage- | G | M | G | M | — | V | — | T | — | — | — | T | — | M | 0.0799 | 1.6172 | 0.0797 | 3895 |
| 3579 | | | | | | | | | | | | | | | | | | |
| Phage- | Q | L | A | M | — | V | — | L | — | — | — | Y | — | — | 0.0703 | 1.6168 | 0.092 | 3896 |
| 3580 | | | | | | | | | | | | | | | | | | |
| Phage- | L | G | H | W | — | S | — | T | — | — | — | M | — | — | 0.1009 | 1.6157 | 0.1101 | 3897 |
| 3581 | | | | | | | | | | | | | | | | | | |
| Phage- | E | S | Y | T | — | V | — | M | — | — | — | T | — | — | 0.0683 | 1.613 | 0.088 | 3898 |
| 3582 | | | | | | | | | | | | | | | | | | |
| Phage- | N | I | M | R | — | — | — | S | — | — | — | T | — | — | 0.0546 | 1.6125 | 0.064 | 3899 |
| 3583 | | | | | | | | | | | | | | | | | | |
| Phage- | P | S | P | I | — | R | — | E | — | — | — | V | W | L | 0.0681 | 1.6111 | 0.0954 | 3900 |
| 3584 | | | | | | | | | | | | | | | | | | |
| Phage- | T | F | M | R | — | E | — | V | — | — | — | D | — | — | 0.0593 | 1.6098 | 0.0709 | 3901 |
| 3585 | | | | | | | | | | | | | | | | | | |
| Phage- | N | L | G | W | — | V | — | V | — | — | — | E | — | — | 0.1094 | 1.6066 | 0.1012 | 3902 |
| 3586 | | | | | | | | | | | | | | | | | | |
| Phage- | H | E | P | V | — | R | — | E | — | — | — | Q | — | — | 0.0621 | 1.6011 | 0.0717 | 3903 |
| 3587 | | | | | | | | | | | | | | | | | | |
| Phage- | E | S | H | — | — | R | — | A | — | — | — | T | — | — | 0.0776 | 1.6004 | 0.089 | 3904 |
| 3588 | | | | | | | | | | | | | | | | | | |
| Phage- | T | T | L | M | — | T | — | Q | — | — | — | D | — | — | 0.0696 | 1.6 | 0.0718 | 3905 |
| 3589 | | | | | | | | | | | | | | | | | | |
| Phage- | M | P | G | A | — | V | — | L | — | — | — | Y | — | — | 0.0598 | 1.5998 | 0.0685 | 3906 |
| 3590 | | | | | | | | | | | | | | | | | | |
| Phage- | E | E | L | M | — | R | — | V | — | — | — | L | — | — | 0.0848 | 1.5989 | 0.0789 | 3907 |
| 3591 | | | | | | | | | | | | | | | | | | |
| Phage- | V | E | K | M | — | K | — | A | — | — | — | N | V | — | 0.0635 | 1.5979 | 0.0777 | 3908 |
| 3592 | | | | | | | | | | | | | | | | | | |
| Phage- | P | D | P | — | — | V | — | — | — | — | — | I | — | L | 0.0718 | 1.5962 | 0.0686 | 3909 |
| 3593 | | | | | | | | | | | | | | | | | | |
| Phage- | T | E | I | R | — | — | — | Q | — | — | — | H | — | — | 0.0593 | 1.5953 | 0.0698 | 3910 |
| 3594 | | | | | | | | | | | | | | | | | | |
| Phage- | Q | G | — | A | — | I | — | T | — | — | — | T | — | — | 0.0617 | 1.595 | 0.0711 | 3911 |
| 3595 | | | | | | | | | | | | | | | | | | |
| Phage- | L | E | R | — | — | R | — | A | — | — | — | D | — | — | 0.0751 | 1.5874 | 0.1356 | 3912 |
| 3596 | | | | | | | | | | | | | | | | | | |
| Phage- | E | I | P | W | — | V | — | S | — | — | — | N | Y | — | 0.0848 | 1.587 | 0.0904 | 3913 |
| 3597 | | | | | | | | | | | | | | | | | | |
| Phage- | V | E | T | — | — | R | — | A | — | — | — | W | — | — | 0.0727 | 1.5864 | 0.0788 | 3914 |
| 3598 | | | | | | | | | | | | | | | | | | |
| Phage- | Q | D | L | K | — | V | — | S | — | — | — | S | — | — | 0.0672 | 1.5852 | 0.0713 | 3915 |
| 3599 | | | | | | | | | | | | | | | | | | |
| Phage- | W | E | — | R | — | E | — | A | — | — | — | I | — | L | 0.0701 | 1.5851 | 0.0694 | 3916 |
| 3600 | | | | | | | | | | | | | | | | | | |
| Phage- | V | M | H | R | — | E | — | S | — | — | — | W | — | — | 0.0751 | 1.585 | 0.1183 | 3917 |
| 3601 | | | | | | | | | | | | | | | | | | |
| Phage- | H | E | P | V | — | K | — | S | — | — | — | T | — | L | 0.0659 | 1.5848 | 0.0595 | 3918 |
| 3602 | | | | | | | | | | | | | | | | | | |
| Phage- | E | G | L | R | — | V | — | A | — | — | — | — | — | — | 0.0642 | 1.5841 | 0.0633 | 3919 |
| 3603 | | | | | | | | | | | | | | | | | | |
| Phage- | H | V | A | M | — | — | — | T | — | — | — | M | — | — | 0.0845 | 1.5835 | 0.1235 | 3920 |
| 3604 | | | | | | | | | | | | | | | | | | |
| Phage- | W | T | F | K | — | E | — | V | — | — | — | E | — | — | 0.0778 | 1.5827 | 0.1008 | 3921 |
| 3605 | | | | | | | | | | | | | | | | | | |
| Phage- | V | V | — | S | — | T | — | S | — | — | — | T | — | L | 0.0673 | 1.5826 | 0.0718 | 3922 |
| 3606 | | | | | | | | | | | | | | | | | | |
| Phage- | L | D | I | V | — | R | — | S | — | — | — | Y | — | — | 0.0683 | 1.5813 | 0.0774 | 3923 |
| 3607 | | | | | | | | | | | | | | | | | | |
| Phage- | E | T | — | R | — | I | — | S | — | — | — | T | — | L | 0.0668 | 1.5791 | 0.0717 | 3924 |
| 3608 | | | | | | | | | | | | | | | | | | |
| Phage- | S | H | M | V | — | K | — | T | — | — | — | Q | — | — | 0.1175 | 1.5775 | 0.074 | 3925 |
| 3609 | | | | | | | | | | | | | | | | | | |
| Phage- | V | Q | P | A | — | V | — | I | — | — | — | D | — | L | 0.0538 | 1.5774 | 0.0782 | 3926 |
| 3610 | | | | | | | | | | | | | | | | | | |
| Phage- | V | D | R | — | — | M | — | I | — | — | — | E | — | L | 0.0616 | 1.5766 | 0.0788 | 3927 |
| 3611 | | | | | | | | | | | | | | | | | | |
| Phage- | D | P | — | M | — | M | — | R | — | — | — | D | — | — | 0.0741 | 1.5744 | 0.0916 | 3928 |
| 3612 | | | | | | | | | | | | | | | | | | |
| Phage- | E | T | M | R | — | E | — | S | — | — | — | I | — | L | 0.0564 | 1.5712 | 0.0724 | 3929 |
| 3613 | | | | | | | | | | | | | | | | | | |
| Phage- | G | W | E | W | — | I | — | E | — | — | — | T | — | L | 0.0613 | 1.5706 | 0.0625 | 3930 |
| 3614 | | | | | | | | | | | | | | | | | | |
| Phage- | L | D | R | — | — | L | — | Q | — | — | — | V | — | M | 0.0587 | 1.5688 | 0.0704 | 3931 |
| 3615 | | | | | | | | | | | | | | | | | | |
| Phage- | E | S | R | — | — | V | — | M | — | — | — | S | — | — | 0.0597 | 1.5688 | 0.0707 | 3932 |
| 3616 | | | | | | | | | | | | | | | | | | |
| Phage- | D | F | I | R | — | S | — | I | — | — | — | D | — | — | 0.0577 | 1.5682 | 0.0807 | 3933 |
| 3617 | | | | | | | | | | | | | | | | | | |
| Phage- | V | V | A | V | — | K | — | L | — | — | — | E | A | — | 0.0521 | 1.5679 | 0.062 | 3934 |
| 3618 | | | | | | | | | | | | | | | | | | |
| Phage- | A | D | H | M | — | V | — | Q | — | — | — | S | — | — | 0.0597 | 1.5675 | 0.1027 | 3935 |
| 3619 | | | | | | | | | | | | | | | | | | |
| Phage- | D | E | R | M | — | V | — | E | — | — | — | M | — | M | 0.0603 | 1.567 | 0.0714 | 3936 |
| 3620 | | | | | | | | | | | | | | | | | | |
| Phage- | H | D | Y | A | — | V | — | K | — | — | — | E | — | L | 0.0575 | 1.5659 | 0.0818 | 3937 |
| 3621 | | | | | | | | | | | | | | | | | | |
| Phage- | Y | M | M | K | — | I | — | E | — | — | — | T | — | L | 0.0649 | 1.5574 | 0.0662 | 3938 |
| 3622 | | | | | | | | | | | | | | | | | | |
| Phage- | — | V | K | F | — | I | — | Q | — | — | — | D | — | — | 0.0941 | 1.5574 | 0.1475 | 3939 |
| 3623 | | | | | | | | | | | | | | | | | | |
| Phage- | D | D | R | V | — | R | — | D | — | — | — | N | — | — | 0.0655 | 1.5558 | 0.0665 | 3940 |
| 3624 | | | | | | | | | | | | | | | | | | |
| Phage- | D | W | — | M | — | K | — | E | — | — | — | — | — | — | 0.0753 | 1.554 | 0.0618 | 3941 |
| 3625 | | | | | | | | | | | | | | | | | | |
| Phage- | A | G | Y | A | — | V | — | S | — | — | — | E | — | — | 0.0589 | 1.5527 | 0.105 | 3942 |
| 3626 | | | | | | | | | | | | | | | | | | |
| Phage- | L | — | — | K | — | E | — | A | — | — | — | T | — | — | 0.0675 | 1.5514 | 0.0802 | 3943 |
| 3627 | | | | | | | | | | | | | | | | | | |
| Phage- | V | H | P | M | — | T | — | L | — | — | — | V | — | — | 0.0797 | 1.5501 | 0.0993 | 3944 |
| 3628 | | | | | | | | | | | | | | | | | | |
| Phage- | D | L | M | M | — | V | — | A | — | — | — | E | — | — | 0.0749 | 1.5495 | 0.0929 | 3945 |
| 3629 | | | | | | | | | | | | | | | | | | |
| Phage- | E | L | H | — | — | I | — | S | — | — | — | T | — | L | 0.0595 | 1.5434 | 0.1036 | 3946 |
| 3630 | | | | | | | | | | | | | | | | | | |
| Phage- | L | V | N | Y | — | I | — | P | — | — | — | — | — | — | 0.0716 | 1.5425 | 0.093 | 3947 |
| 3631 | | | | | | | | | | | | | | | | | | |
| Phage- | E | L | I | — | — | R | — | S | — | — | — | T | — | L | 0.0712 | 1.5423 | 0.0672 | 3948 |
| 3632 | | | | | | | | | | | | | | | | | | |
| Phage- | S | H | A | — | — | R | — | A | — | — | — | M | — | L | 0.0619 | 1.5389 | 0.0783 | 3949 |
| 3633 | | | | | | | | | | | | | | | | | | |
| Phage- | G | T | T | W | — | V | — | T | — | — | — | E | — | L | 0.0551 | 1.5385 | 0.077 | 3950 |
| 3634 | | | | | | | | | | | | | | | | | | |
| Phage- | E | V | A | M | — | K | — | L | — | — | — | S | — | — | 0.0643 | 1.5333 | 0.069 | 3951 |
| 3635 | | | | | | | | | | | | | | | | | | |
| Phage- | V | E | P | — | — | R | — | T | — | — | — | M | S | L | 0.0672 | 1.5321 | 0.0766 | 3952 |
| 3636 | | | | | | | | | | | | | | | | | | |
| Phage- | A | E | — | K | — | I | — | T | — | — | — | T | — | — | 0.0628 | 1.53 | 0.079 | 3953 |
| 3637 | | | | | | | | | | | | | | | | | | |
| Phage- | V | H | Q | — | — | I | — | V | — | — | — | S | — | — | 0.076 | 1.5299 | 0.0996 | 3954 |
| 3638 | | | | | | | | | | | | | | | | | | |
| Phage- | Y | P | P | — | — | V | — | M | — | — | — | N | — | — | 0.0766 | 1.5298 | 0.1071 | 3955 |
| 3639 | | | | | | | | | | | | | | | | | | |
| Phage- | F | P | A | M | — | K | — | S | — | — | — | T | — | — | 0.0643 | 1.528 | 0.082 | 3956 |
| 3640 | | | | | | | | | | | | | | | | | | |
| Phage- | S | N | M | V | — | I | — | S | — | — | — | N | — | — | 0.061 | 1.5275 | 0.0754 | 3957 |
| 3641 | | | | | | | | | | | | | | | | | | |
| Phage- | V | T | M | K | — | — | — | M | — | — | — | Q | — | L | 0.0622 | 1.5235 | 0.0738 | 3958 |
| 3642 | | | | | | | | | | | | | | | | | | |
| Phage- | A | D | R | V | — | K | — | A | — | — | — | D | L | — | 0.0529 | 1.5191 | 0.0527 | 3959 |
| 3643 | | | | | | | | | | | | | | | | | | |
| Phage- | P | N | I | — | — | K | — | Q | — | — | — | — | — | — | 0.0646 | 1.5188 | 0.0825 | 3960 |
| 3644 | | | | | | | | | | | | | | | | | | |
| Phage- | V | D | — | R | — | M | — | L | — | — | — | S | — | — | 0.055 | 1.5157 | 0.0628 | 3961 |
| 3645 | | | | | | | | | | | | | | | | | | |
| Phage- | T | — | — | M | — | I | — | Y | — | — | — | G | — | M | 0.0719 | 1.5152 | 0.104 | 3962 |
| 3646 | | | | | | | | | | | | | | | | | | |
| Phage- | M | V | P | V | — | I | — | L | — | — | — | S | — | — | 0.0844 | 1.5093 | 0.0744 | 3963 |
| 3647 | | | | | | | | | | | | | | | | | | |
| Phage- | P | L | — | — | — | I | — | Q | — | — | — | Q | A | M | 0.0728 | 1.5051 | 0.0663 | 3964 |
| 3648 | | | | | | | | | | | | | | | | | | |
| Phage- | L | Q | T | — | — | I | — | P | — | — | — | M | — | — | 0.0621 | 1.5046 | 0.0685 | 3965 |
| 3649 | | | | | | | | | | | | | | | | | | |
| Phage- | E | Q | — | — | — | R | — | A | — | — | — | L | — | — | 0.0685 | 1.5036 | 0.0974 | 3966 |
| 3650 | | | | | | | | | | | | | | | | | | |
| Phage- | S | V | — | M | — | V | — | E | — | — | — | Q | — | — | 0.0618 | 1.5034 | 0.0734 | 3967 |
| 3651 | | | | | | | | | | | | | | | | | | |
| Phage- | P | S | A | V | — | V | — | T | — | — | — | S | — | — | 0.058 | 1.5027 | 0.0815 | 3968 |
| 3652 | | | | | | | | | | | | | | | | | | |
| Phage- | V | Q | I | — | — | R | — | Q | — | — | — | E | — | L | 0.055 | 1.5003 | 0.0669 | 3969 |
| 3653 | | | | | | | | | | | | | | | | | | |
| Phage- | L | — | L | A | — | V | — | P | — | — | — | Q | — | L | 0.069 | 1.5002 | 0.0897 | 3970 |
| 3654 | | | | | | | | | | | | | | | | | | |
| Phage- | V | E | R | M | — | E | — | L | — | — | — | — | — | L | 0.0721 | 1.4985 | 0.0832 | 3971 |
| 3655 | | | | | | | | | | | | | | | | | | |
| Phage- | E | T | S | W | — | T | — | S | — | — | — | T | — | — | 0.0721 | 1.4978 | 0.0752 | 3972 |
| 3656 | | | | | | | | | | | | | | | | | | |
| Phage- | S | M | I | Y | — | I | — | S | — | — | — | E | — | M | 0.0855 | 1.4974 | 0.0844 | 3973 |
| 3657 | | | | | | | | | | | | | | | | | | |
| Phage- | — | D | R | H | — | T | — | S | — | — | — | Y | — | — | 0.0557 | 1.4962 | 0.0562 | 3974 |
| 3658 | | | | | | | | | | | | | | | | | | |
| Phage- | D | H | K | — | — | K | — | E | — | — | — | — | — | L | 0.0514 | 1.4951 | 0.0672 | 3975 |
| 3659 | | | | | | | | | | | | | | | | | | |
| Phage- | M | V | S | H | — | V | — | L | — | — | — | — | — | — | 0.0692 | 1.4941 | 0.0616 | 3976 |
| 3660 | | | | | | | | | | | | | | | | | | |
| Phage- | V | — | I | A | — | T | — | M | — | — | — | T | — | — | 0.072 | 1.4938 | 0.113 | 3977 |
| 3661 | | | | | | | | | | | | | | | | | | |
| Phage- | H | V | A | A | — | V | — | S | — | — | — | T | — | — | 0.0655 | 1.4911 | 0.0842 | 3978 |
| 3662 | | | | | | | | | | | | | | | | | | |
| Phage- | E | G | P | — | — | K | — | A | — | — | — | R | — | — | 0.0538 | 1.4904 | 0.0963 | 3979 |
| 3663 | | | | | | | | | | | | | | | | | | |
| Phage- | P | V | P | Y | — | R | — | I | — | — | — | Q | — | L | 0.069 | 1.4891 | 0.0736 | 3980 |
| 3664 | | | | | | | | | | | | | | | | | | |
| Phage- | E | S | R | F | — | V | — | A | — | — | — | — | A | V | 0.0622 | 1.4882 | 0.089 | 3981 |
| 3665 | | | | | | | | | | | | | | | | | | |
| Phage- | E | V | R | R | — | E | — | S | — | — | — | S | V | V | 0.0583 | 1.4877 | 0.0749 | 3982 |
| 3666 | | | | | | | | | | | | | | | | | | |
| Phage- | Y | I | Q | A | — | K | — | R | — | — | — | N | — | — | 0.0737 | 1.4861 | 0.0874 | 3983 |
| 3667 | | | | | | | | | | | | | | | | | | |
| Phage- | L | Q | — | — | — | V | — | D | — | — | — | — | — | — | 0.085 | 1.4859 | 0.091 | 3984 |
| 3668 | | | | | | | | | | | | | | | | | | |
| Phage- | G | H | — | Y | — | E | — | Q | — | — | — | Y | — | — | 0.0582 | 1.4858 | 0.0791 | 3985 |
| 3669 | | | | | | | | | | | | | | | | | | |
| Phage- | P | E | L | R | — | H | — | P | — | — | — | T | — | — | 0.0762 | 1.4858 | 0.0997 | 3986 |
| 3670 | | | | | | | | | | | | | | | | | | |
| Phage- | — | S | I | K | — | E | — | Q | — | — | — | D | — | L | 0.0747 | 1.4839 | 0.3069 | 3987 |
| 3671 | | | | | | | | | | | | | | | | | | |
| Phage- | A | — | K | Y | — | E | — | S | — | — | — | Y | — | — | 0.0791 | 1.4834 | 0.1047 | 3988 |
| 3672 | | | | | | | | | | | | | | | | | | |
| Phage- | L | G | K | I | — | S | — | A | — | — | — | S | — | — | 0.0603 | 1.4806 | 0.0608 | 3989 |
| 3673 | | | | | | | | | | | | | | | | | | |
| Phage- | G | T | M | K | — | E | — | A | — | — | — | E | — | — | 0.0762 | 1.4805 | 0.1056 | 3990 |
| 3674 | | | | | | | | | | | | | | | | | | |
| Phage- | D | — | M | M | — | K | — | T | — | — | — | D | — | — | 0.0567 | 1.4782 | 0.0662 | 3991 |
| 3675 | | | | | | | | | | | | | | | | | | |
| Phage- | P | E | A | R | — | I | — | M | — | — | — | E | R | L | 0.056 | 1.4779 | 0.0885 | 3992 |
| 3676 | | | | | | | | | | | | | | | | | | |
| Phage- | L | V | K | H | — | V | — | D | — | — | — | S | — | — | 0.0846 | 1.4763 | 0.1006 | 3993 |
| 3677 | | | | | | | | | | | | | | | | | | |
| Phage- | V | I | P | K | — | E | — | K | — | — | — | H | S | L | 0.0563 | 1.4752 | 0.0649 | 3994 |
| 3678 | | | | | | | | | | | | | | | | | | |
| Phage- | M | I | K | M | — | K | — | Y | — | — | — | E | A | M | 0.0635 | 1.475 | 0.0794 | 3995 |
| 3679 | | | | | | | | | | | | | | | | | | |
| Phage- | Y | T | — | Q | — | V | — | T | — | — | — | — | — | L | 0.0529 | 1.4723 | 0.0609 | 3996 |
| 3680 | | | | | | | | | | | | | | | | | | |
| Phage- | P | H | — | V | — | I | — | E | — | — | — | — | — | — | 0.0647 | 1.472 | 0.068 | 3997 |
| 3681 | | | | | | | | | | | | | | | | | | |
| Phage- | D | E | R | M | — | R | — | A | — | — | — | E | — | L | 0.0624 | 1.47 | 0.1024 | 3998 |
| 3682 | | | | | | | | | | | | | | | | | | |
| Phage- | S | T | S | W | — | S | — | P | — | — | — | E | — | — | 0.0617 | 1.4692 | 0.0631 | 3999 |
| 3683 | | | | | | | | | | | | | | | | | | |
| Phage- | E | R | P | A | — | V | — | M | — | — | — | T | — | L | 0.0711 | 1.4683 | 0.0929 | 4000 |
| 3684 | | | | | | | | | | | | | | | | | | |
| Phage- | E | M | S | — | — | I | — | A | — | — | — | S | V | — | 0.0786 | 1.468 | 0.0677 | 4001 |
| 3685 | | | | | | | | | | | | | | | | | | |
| Phage- | A | D | M | R | — | V | — | D | — | — | — | H | — | — | 0.0751 | 1.468 | 0.0874 | 4002 |
| 3686 | | | | | | | | | | | | | | | | | | |
| Phage- | M | I | A | M | — | K | — | P | — | — | — | I | — | L | 0.0648 | 1.4678 | 0.0954 | 4003 |
| 3687 | | | | | | | | | | | | | | | | | | |
| Phage- | V | V | — | Q | — | V | — | K | — | — | — | Y | — | — | 0.0573 | 1.4668 | 0.0764 | 4004 |
| 3688 | | | | | | | | | | | | | | | | | | |
| Phage- | L | P | H | A | — | K | — | S | — | — | — | Q | — | — | 0.0553 | 1.4665 | 0.0636 | 4005 |
| 3689 | | | | | | | | | | | | | | | | | | |
| Phage- | A | L | S | M | — | V | — | Q | — | — | — | Q | — | — | 0.0532 | 1.4638 | 0.0597 | 4006 |
| 3690 | | | | | | | | | | | | | | | | | | |
| Phage- | V | N | E | R | — | I | — | D | — | — | — | V | — | — | 0.0903 | 1.4627 | 0.0649 | 4007 |
| 3691 | | | | | | | | | | | | | | | | | | |
| Phage- | D | H | M | M | — | V | — | P | — | — | — | D | — | I | 0.0892 | 1.4595 | 0.1191 | 4008 |
| 3692 | | | | | | | | | | | | | | | | | | |
| Phage- | S | E | H | Y | — | V | — | — | — | — | — | — | — | L | 0.0931 | 1.4576 | 0.0995 | 4009 |
| 3693 | | | | | | | | | | | | | | | | | | |
| Phage- | T | V | P | R | — | V | — | A | — | — | — | N | W | S | 0.0668 | 1.4549 | 0.0917 | 4010 |
| 3694 | | | | | | | | | | | | | | | | | | |
| Phage- | S | Q | H | S | — | I | — | S | — | — | — | N | — | — | 0.0574 | 1.4533 | 0.154 | 4011 |
| 3695 | | | | | | | | | | | | | | | | | | |
| Phage- | T | M | Q | M | — | K | — | A | — | — | — | S | Q | L | 0.0782 | 1.4485 | 0.0765 | 4012 |
| 3696 | | | | | | | | | | | | | | | | | | |
| Phage- | N | E | K | — | — | V | — | T | — | — | — | D | — | L | 0.0514 | 1.4473 | 0.0612 | 4013 |
| 3697 | | | | | | | | | | | | | | | | | | |
| Phage- | D | V | P | Y | — | I | — | S | — | — | — | H | — | — | 0.064 | 1.4468 | 0.2487 | 4014 |
| 3698 | | | | | | | | | | | | | | | | | | |
| Phage- | Y | L | H | E | — | R | — | E | — | — | — | V | — | — | 0.077 | 1.446 | 0.1533 | 4015 |
| 3699 | | | | | | | | | | | | | | | | | | |
| Phage- | G | L | P | Y | — | V | — | S | — | — | — | D | — | — | 0.0683 | 1.4408 | 0.0858 | 4016 |
| 3700 | | | | | | | | | | | | | | | | | | |
| Phage- | E | V | L | — | — | K | — | S | — | — | — | D | — | — | 0.0731 | 1.44 | 0.1855 | 4017 |
| 3701 | | | | | | | | | | | | | | | | | | |
| Phage- | P | G | A | — | — | V | — | F | — | — | — | Q | — | — | 0.0871 | 1.4354 | 0.1084 | 4018 |
| 3702 | | | | | | | | | | | | | | | | | | |
| Phage- | V | P | R | M | — | I | — | H | — | — | — | E | — | I | 0.0623 | 1.4331 | 0.0731 | 4019 |
| 3703 | | | | | | | | | | | | | | | | | | |
| Phage- | G | D | — | R | — | V | — | S | — | — | — | E | — | Y | 0.0804 | 1.4313 | 0.0816 | 4020 |
| 3704 | | | | | | | | | | | | | | | | | | |
| Phage- | M | S | M | S | — | V | — | L | — | — | — | S | — | — | 0.0667 | 1.4308 | 0.0698 | 4021 |
| 3705 | | | | | | | | | | | | | | | | | | |
| Phage- | E | I | P | — | — | R | — | E | — | — | — | T | — | L | 0.0754 | 1.4297 | 0.1264 | 4022 |
| 3706 | | | | | | | | | | | | | | | | | | |
| Phage- | V | D | H | M | — | K | — | M | — | — | — | N | — | — | 0.0584 | 1.4295 | 0.0673 | 4023 |
| 3707 | | | | | | | | | | | | | | | | | | |
| Phage- | T | D | R | V | — | T | — | A | — | — | — | Q | — | L | 0.066 | 1.4286 | 0.0772 | 4024 |
| 3708 | | | | | | | | | | | | | | | | | | |
| Phage- | E | H | I | R | — | — | — | S | — | — | — | W | — | — | 0.086 | 1.4284 | 0.1283 | 4025 |
| 3709 | | | | | | | | | | | | | | | | | | |
| Phage- | E | G | R | Y | — | V | — | D | — | — | — | L | — | L | 0.0654 | 1.426 | 0.0705 | 4026 |
| 3710 | | | | | | | | | | | | | | | | | | |
| Phage- | — | — | F | R | — | V | — | E | — | — | — | S | A | — | 0.0632 | 1.426 | 0.07 | 4027 |
| 3711 | | | | | | | | | | | | | | | | | | |
| Phage- | E | V | W | M | — | R | — | S | — | — | — | D | — | — | 0.0565 | 1.4255 | 0.0717 | 4028 |
| 3712 | | | | | | | | | | | | | | | | | | |
| Phage- | L | E | G | M | — | K | — | A | — | — | — | T | — | — | 0.0655 | 1.4246 | 0.0862 | 4029 |
| 3713 | | | | | | | | | | | | | | | | | | |
| Phage- | G | Q | A | A | — | V | — | S | — | — | — | S | — | — | 0.068 | 1.4243 | 0.0867 | 4030 |
| 3714 | | | | | | | | | | | | | | | | | | |
| Phage- | Y | G | — | Q | — | V | — | S | — | — | — | Q | — | — | 0.0734 | 1.4214 | 0.0735 | 4031 |
| 3715 | | | | | | | | | | | | | | | | | | |
| Phage- | — | E | R | M | — | M | — | P | — | — | — | S | A | L | 0.0641 | 1.4203 | 0.0662 | 4032 |
| 3716 | | | | | | | | | | | | | | | | | | |
| Phage- | M | E | W | H | — | K | — | K | — | — | — | E | — | — | 0.0909 | 1.419 | 0.0772 | 4033 |
| 3717 | | | | | | | | | | | | | | | | | | |
| Phage- | E | S | P | I | — | K | — | S | — | — | — | S | — | L | 0.0536 | 1.4183 | 0.0616 | 4034 |
| 3718 | | | | | | | | | | | | | | | | | | |
| Phage- | S | E | A | — | — | V | — | K | — | — | — | H | — | — | 0.0672 | 1.4159 | 0.0994 | 4035 |
| 3719 | | | | | | | | | | | | | | | | | | |
| Phage- | E | P | R | A | — | V | — | F | — | — | — | L | — | — | 0.0564 | 1.4151 | 0.0796 | 4036 |
| 3720 | | | | | | | | | | | | | | | | | | |
| Phage- | G | E | N | W | — | V | — | Q | — | — | — | D | — | — | 0.0643 | 1.4148 | 0.1388 | 4037 |
| 3721 | | | | | | | | | | | | | | | | | | |
| Phage- | A | Y | M | K | — | S | — | A | — | — | — | E | — | — | 0.0876 | 1.4148 | 0.0811 | 4038 |
| 3722 | | | | | | | | | | | | | | | | | | |
| Phage- | P | Y | A | V | — | V | — | K | — | — | — | S | — | — | 0.0662 | 1.4137 | 0.0805 | 4039 |
| 3723 | | | | | | | | | | | | | | | | | | |
| Phage- | E | E | K | — | — | K | — | A | — | — | — | E | W | — | 0.0593 | 1.4135 | 0.096 | 4040 |
| 3724 | | | | | | | | | | | | | | | | | | |
| Phage- | D | V | L | M | — | T | — | R | — | — | — | D | — | — | 0.0766 | 1.4097 | 0.0822 | 4041 |
| 3725 | | | | | | | | | | | | | | | | | | |
| Phage- | T | L | F | A | — | V | — | A | — | — | — | D | — | — | 0.067 | 1.408 | 0.0757 | 4042 |
| 3726 | | | | | | | | | | | | | | | | | | |
| Phage- | — | D | R | H | — | V | — | R | — | — | — | D | — | L | 0.0637 | 1.4076 | 0.0751 | 4043 |
| 3727 | | | | | | | | | | | | | | | | | | |
| Phage- | E | S | R | — | — | V | — | T | — | — | — | T | W | L | 0.0777 | 1.407 | 0.0946 | 4044 |
| 3728 | | | | | | | | | | | | | | | | | | |
| Phage- | D | Y | S | V | — | R | — | A | — | — | — | Q | — | — | 0.0673 | 1.405 | 0.0699 | 4045 |
| 3729 | | | | | | | | | | | | | | | | | | |
| Phage- | F | T | A | R | — | E | — | S | — | — | — | V | — | — | 0.0587 | 1.405 | 0.0626 | 4046 |
| 3730 | | | | | | | | | | | | | | | | | | |
| Phage- | L | Y | K | E | — | V | — | Q | — | — | — | S | — | — | 0.0716 | 1.4038 | 0.0798 | 4047 |
| 3731 | | | | | | | | | | | | | | | | | | |
| Phage- | T | G | M | A | — | V | — | W | — | — | — | E | — | — | 0.0643 | 1.4025 | 0.0812 | 4048 |
| 3732 | | | | | | | | | | | | | | | | | | |
| Phage- | A | D | — | R | — | V | — | D | — | — | — | W | — | M | 0.0578 | 1.4004 | 0.0602 | 4049 |
| 3733 | | | | | | | | | | | | | | | | | | |
| Phage- | E | L | P | V | — | R | — | S | — | — | — | G | — | L | 0.0592 | 1.3992 | 0.0603 | 4050 |
| 3734 | | | | | | | | | | | | | | | | | | |
| Phage- | Q | V | K | Y | — | I | — | E | — | — | — | S | S | — | 0.074 | 1.3989 | 0.0671 | 4051 |
| 3735 | | | | | | | | | | | | | | | | | | |
| Phage- | F | T | A | Q | — | V | — | L | — | — | — | L | — | — | 0.0749 | 1.3943 | 0.0789 | 4052 |
| 3736 | | | | | | | | | | | | | | | | | | |
| Phage- | V | S | H | H | — | V | — | A | — | — | — | F | — | L | 0.0802 | 1.3941 | 0.0946 | 4053 |
| 3737 | | | | | | | | | | | | | | | | | | |
| Phage- | H | S | — | H | — | V | — | M | — | — | — | T | — | — | 0.0596 | 1.3913 | 0.0781 | 4054 |
| 3738 | | | | | | | | | | | | | | | | | | |
| Phage- | V | N | R | M | — | V | — | V | — | — | — | Q | — | — | 0.063 | 1.3913 | 0.0729 | 4055 |
| 3739 | | | | | | | | | | | | | | | | | | |
| Phage- | L | L | L | R | — | E | — | T | — | — | — | T | — | — | 0.0809 | 1.3901 | 0.0711 | 4056 |
| 3740 | | | | | | | | | | | | | | | | | | |
| Phage- | W | T | P | — | — | K | — | L | — | — | — | Q | — | L | 0.1544 | 1.3874 | 0.1596 | 4057 |
| 3741 | | | | | | | | | | | | | | | | | | |
| Phage- | Y | S | I | S | — | V | — | Q | — | — | — | E | — | — | 0.0544 | 1.3874 | 0.0591 | 4058 |
| 3742 | | | | | | | | | | | | | | | | | | |
| Phage- | V | Q | P | W | — | V | — | S | — | — | — | E | R | — | 0.0593 | 1.3872 | 0.3118 | 4059 |
| 3743 | | | | | | | | | | | | | | | | | | |
| Phage- | P | G | K | V | — | E | — | I | — | — | — | L | — | — | 0.1081 | 1.3845 | 0.1237 | 4060 |
| 3744 | | | | | | | | | | | | | | | | | | |
| Phage- | W | I | K | E | — | V | — | D | — | — | — | S | — | — | 0.0707 | 1.3834 | 0.0803 | 4061 |
| 3745 | | | | | | | | | | | | | | | | | | |
| Phage- | V | M | P | R | — | E | — | I | — | — | — | R | — | — | 0.0568 | 1.3833 | 0.0701 | 4062 |
| 3746 | | | | | | | | | | | | | | | | | | |
| Phage- | M | E | P | V | — | L | — | E | — | — | — | W | — | — | 0.0596 | 1.3804 | 0.0902 | 4063 |
| 3747 | | | | | | | | | | | | | | | | | | |
| Phage- | Q | S | — | M | — | R | — | A | — | — | — | E | — | — | 0.0578 | 1.3795 | 0.0813 | 4064 |
| 3748 | | | | | | | | | | | | | | | | | | |
| Phage- | V | P | A | — | — | V | — | — | — | — | — | S | — | L | 0.0602 | 1.3792 | 0.0565 | 4065 |
| 3749 | | | | | | | | | | | | | | | | | | |
| Phage- | E | Q | P | A | — | V | — | K | — | — | — | I | — | — | 0.0758 | 1.3773 | 0.0893 | 4066 |
| 3750 | | | | | | | | | | | | | | | | | | |
| Phage- | E | T | P | R | — | V | — | W | — | — | — | I | D | I | 0.0595 | 1.3745 | 0.0731 | 4067 |
| 3751 | | | | | | | | | | | | | | | | | | |
| Phage- | N | S | S | — | — | I | — | S | — | — | — | S | — | — | 0.064 | 1.3737 | 0.0738 | 4068 |
| 3752 | | | | | | | | | | | | | | | | | | |
| Phage- | F | E | L | Y | — | V | — | A | — | — | — | S | — | — | 0.0638 | 1.3707 | 0.1596 | 4069 |
| 3753 | | | | | | | | | | | | | | | | | | |
| Phage- | E | — | K | V | — | K | — | A | — | — | — | D | — | L | 0.077 | 1.3707 | 0.077 | 4070 |
| 3754 | | | | | | | | | | | | | | | | | | |
| Phage- | E | E | M | M | — | K | — | D | — | — | — | Y | — | — | 0.0963 | 1.3698 | 0.1086 | 4071 |
| 3755 | | | | | | | | | | | | | | | | | | |
| Phage- | Y | H | K | E | — | I | — | P | — | — | — | M | — | — | 0.0939 | 1.3693 | 0.0922 | 4072 |
| 3756 | | | | | | | | | | | | | | | | | | |
| Phage- | T | I | M | Y | — | K | — | T | — | — | — | T | — | — | 0.0626 | 1.368 | 0.0727 | 4073 |
| 3757 | | | | | | | | | | | | | | | | | | |
| Phage- | Y | E | T | V | — | K | — | L | — | — | — | T | — | — | 0.0546 | 1.3636 | 0.0806 | 4074 |
| 3758 | | | | | | | | | | | | | | | | | | |
| Phage- | D | V | G | M | — | R | — | K | — | — | — | D | — | — | 0.0647 | 1.3621 | 0.0713 | 4075 |
| 3759 | | | | | | | | | | | | | | | | | | |
| Phage- | V | L | P | S | — | I | — | S | — | — | — | N | — | — | 0.067 | 1.362 | 0.0834 | 4076 |
| 3760 | | | | | | | | | | | | | | | | | | |
| Phage- | E | V | — | A | — | V | — | E | — | — | — | V | — | — | 0.0681 | 1.3607 | 0.102 | 4077 |
| 3761 | | | | | | | | | | | | | | | | | | |
| Phage- | L | V | H | — | — | K | — | P | — | — | — | H | — | — | 0.0903 | 1.3605 | 0.1227 | 4078 |
| 3762 | | | | | | | | | | | | | | | | | | |
| Phage- | D | Q | R | E | — | I | — | H | — | — | — | — | — | — | 0.0744 | 1.3591 | 0.064 | 4079 |
| 3763 | | | | | | | | | | | | | | | | | | |
| Phage- | E | N | R | M | — | I | — | E | — | — | — | Q | A | S | 0.0813 | 1.3557 | 0.0737 | 4080 |
| 3764 | | | | | | | | | | | | | | | | | | |
| Phage- | M | V | I | E | — | R | — | E | — | — | — | V | — | — | 0.0596 | 1.3546 | 0.0624 | 4081 |
| 3765 | | | | | | | | | | | | | | | | | | |
| Phage- | L | Y | G | V | — | V | — | E | — | — | — | H | — | — | 0.0797 | 1.3541 | 0.0728 | 4082 |
| 3766 | | | | | | | | | | | | | | | | | | |
| Phage- | V | V | R | V | — | E | — | S | — | — | — | — | — | — | 0.0665 | 1.3535 | 0.0848 | 4083 |
| 3767 | | | | | | | | | | | | | | | | | | |
| Phage- | S | E | P | V | — | T | — | M | — | — | — | T | — | I | 0.0561 | 1.3519 | 0.0611 | 4084 |
| 3768 | | | | | | | | | | | | | | | | | | |
| Phage- | G | S | P | F | — | V | — | L | — | — | — | E | — | — | 0.0708 | 1.3513 | 0.0805 | 4085 |
| 3769 | | | | | | | | | | | | | | | | | | |
| Phage- | T | T | P | M | — | V | — | A | — | — | — | S | — | M | 0.0569 | 1.3439 | 0.0706 | 4086 |
| 3770 | | | | | | | | | | | | | | | | | | |
| Phage- | G | N | I | W | — | E | — | A | — | — | — | V | — | — | 0.0555 | 1.3411 | 0.0639 | 4087 |
| 3771 | | | | | | | | | | | | | | | | | | |
| Phage- | G | L | R | M | — | E | — | M | — | — | — | Q | — | — | 0.0783 | 1.3369 | 0.0749 | 4088 |
| 3772 | | | | | | | | | | | | | | | | | | |
| Phage- | V | H | R | M | — | E | — | K | — | — | — | E | — | — | 0.0752 | 1.3344 | 0.0695 | 4089 |
| 3773 | | | | | | | | | | | | | | | | | | |
| Phage- | W | E | G | F | — | V | — | T | — | — | — | D | — | — | 0.0642 | 1.3342 | 0.0864 | 4090 |
| 3774 | | | | | | | | | | | | | | | | | | |
| Phage- | V | Q | L | M | — | M | — | E | — | — | — | S | — | L | 0.0534 | 1.3339 | 0.0599 | 4091 |
| 3775 | | | | | | | | | | | | | | | | | | |
| Phage- | P | V | Q | — | — | I | — | S | — | — | — | D | — | — | 0.0574 | 1.3335 | 0.0876 | 4092 |
| 3776 | | | | | | | | | | | | | | | | | | |
| Phage- | — | S | P | I | — | R | — | A | — | — | — | H | — | — | 0.0616 | 1.332 | 0.0873 | 4093 |
| 3777 | | | | | | | | | | | | | | | | | | |
| Phage- | V | T | T | — | — | R | — | A | — | — | — | H | — | — | 0.0662 | 1.3307 | 0.0881 | 4094 |
| 3778 | | | | | | | | | | | | | | | | | | |
| Phage- | E | Y | Q | R | — | I | — | E | — | — | — | S | — | — | 0.0989 | 1.3304 | 0.0735 | 4095 |
| 3779 | | | | | | | | | | | | | | | | | | |
| Phage- | H | M | p | — | — | I | — | V | — | — | — | — | — | — | 0.1195 | 1.3299 | 0.1129 | 4096 |
| 3780 | | | | | | | | | | | | | | | | | | |
| Phage- | L | V | A | R | — | V | — | E | — | — | — | — | — | — | 0.0616 | 1.3278 | 0.1015 | 4097 |
| 3781 | | | | | | | | | | | | | | | | | | |
| Phage- | Y | S | N | F | — | V | — | H | — | — | — | S | — | — | 0.0649 | 1.3272 | 0.0679 | 4098 |
| 3782 | | | | | | | | | | | | | | | | | | |
| Phage- | V | E | — | R | — | V | — | A | — | — | — | E | — | L | 0.0676 | 1.3269 | 0.0698 | 4099 |
| 3783 | | | | | | | | | | | | | | | | | | |
| Phage- | P | G | A | S | — | V | — | A | — | — | — | D | — | — | 0.0616 | 1.3268 | 0.0653 | 4100 |
| 3784 | | | | | | | | | | | | | | | | | | |
| Phage- | S | V | — | K | — | V | — | S | — | — | — | T | — | — | 0.0564 | 1.3238 | 0.0729 | 4101 |
| 3785 | | | | | | | | | | | | | | | | | | |
| Phage- | E | M | I | R | — | E | — | R | — | — | — | S | — | — | 0.0722 | 1.3225 | 0.0656 | 4102 |
| 3786 | | | | | | | | | | | | | | | | | | |
| Phage- | L | K | — | E | — | R | — | L | — | — | — | E | — | — | 0.0651 | 1.3217 | 0.075 | 4103 |
| 3787 | | | | | | | | | | | | | | | | | | |
| Phage- | M | L | R | S | — | V | — | P | — | — | — | — | — | — | 0.0681 | 1.3206 | 0.0821 | 4104 |
| 3788 | | | | | | | | | | | | | | | | | | |
| Phage- | E | Q | — | — | — | R | — | E | — | — | — | I | — | — | 0.0715 | 1.32 | 0.0856 | 4105 |
| 3789 | | | | | | | | | | | | | | | | | | |
| Phage- | V | S | Q | — | — | I | — | E | — | — | — | I | — | I | 0.0588 | 1.3191 | 0.08 | 4106 |
| 3790 | | | | | | | | | | | | | | | | | | |
| Phage- | V | H | M | A | — | T | — | W | — | — | — | V | — | — | 0.077 | 1.3166 | 0.0962 | 4107 |
| 3791 | | | | | | | | | | | | | | | | | | |
| Phage- | H | M | Q | Y | — | I | — | Q | — | — | — | H | — | — | 0.0747 | 1.316 | 0.0825 | 4108 |
| 3792 | | | | | | | | | | | | | | | | | | |
| Phage- | F | — | F | D | — | V | — | K | — | — | — | T | — | — | 0.0574 | 1.3143 | 0.0829 | 4109 |
| 3793 | | | | | | | | | | | | | | | | | | |
| Phage- | T | V | K | Q | — | V | — | E | — | — | — | V | M | L | 0.0756 | 1.313 | 0.1041 | 4110 |
| 3794 | | | | | | | | | | | | | | | | | | |
| Phage- | D | L | I | Y | — | R | — | A | — | — | — | D | — | — | 0.0597 | 1.3116 | 0.0679 | 4111 |
| 3795 | | | | | | | | | | | | | | | | | | |
| Phage- | M | E | R | S | — | I | — | D | — | — | — | L | — | — | 0.0566 | 1.3105 | 0.0541 | 4112 |
| 3796 | | | | | | | | | | | | | | | | | | |
| Phage- | V | E | Y | — | — | K | — | V | — | — | — | Q | — | — | 0.0541 | 1.3102 | 0.0609 | 4113 |
| 3797 | | | | | | | | | | | | | | | | | | |
| Phage- | M | S | M | Y | — | V | — | T | — | — | — | N | — | — | 0.0527 | 1.3099 | 0.0602 | 4114 |
| 3798 | | | | | | | | | | | | | | | | | | |
| Phage- | L | E | L | A | — | R | — | M | — | — | — | Q | — | — | 0.0657 | 1.3059 | 0.0709 | 4115 |
| 3799 | | | | | | | | | | | | | | | | | | |
| Phage- | L | E | L | — | — | R | — | S | — | — | — | E | — | — | 0.057 | 1.3036 | 0.0665 | 4116 |
| 3800 | | | | | | | | | | | | | | | | | | |
| Phage- | E | W | L | V | — | K | — | A | — | — | — | L | — | L | 0.0606 | 1.3032 | 0.0609 | 4117 |
| 3801 | | | | | | | | | | | | | | | | | | |
| Phage- | E | V | L | R | — | V | — | R | — | — | — | E | K | — | 0.0551 | 1.3027 | 0.0658 | 4118 |
| 3802 | | | | | | | | | | | | | | | | | | |
| Phage- | G | G | P | M | — | K | — | V | — | — | — | Q | — | — | 0.0654 | 1.2987 | 0.0735 | 4119 |
| 3803 | | | | | | | | | | | | | | | | | | |
| Phage- | — | R | A | — | — | E | — | S | — | — | — | T | — | — | 0.0641 | 1.2987 | 0.0766 | 4120 |
| 3804 | | | | | | | | | | | | | | | | | | |
| Phage- | H | M | I | H | — | V | — | D | — | — | — | E | — | — | 0.0698 | 1.2956 | 0.0847 | 4121 |
| 3805 | | | | | | | | | | | | | | | | | | |
| Phage- | D | Q | A | A | — | V | — | A | — | — | — | T | — | — | 0.0552 | 1.2954 | 0.1175 | 4122 |
| 3806 | | | | | | | | | | | | | | | | | | |
| Phage- | V | V | S | H | — | K | — | I | — | — | — | S | — | — | 0.0754 | 1.2947 | 0.083 | 4123 |
| 3807 | | | | | | | | | | | | | | | | | | |
| Phage- | L | G | — | Q | — | K | — | T | — | — | — | Q | — | — | 0.0613 | 1.2945 | 0.0911 | 4124 |
| 3808 | | | | | | | | | | | | | | | | | | |
| Phage- | D | — | R | — | — | K | — | T | — | — | — | M | — | L | 0.0651 | 1.2934 | 0.0886 | 4125 |
| 3809 | | | | | | | | | | | | | | | | | | |
| Phage- | G | D | Q | M | — | I | — | L | — | — | — | E | — | — | 0.0845 | 1.2922 | 0.1064 | 4126 |
| 3810 | | | | | | | | | | | | | | | | | | |
| Phage- | S | F | Q | R | — | E | — | E | — | — | — | Y | — | — | 0.0726 | 1.2906 | 0.0768 | 4127 |
| 3811 | | | | | | | | | | | | | | | | | | |
| Phage- | A | Y | P | M | — | S | — | A | — | — | — | — | — | — | 0.0655 | 1.2904 | 0.0779 | 4128 |
| 3812 | | | | | | | | | | | | | | | | | | |
| Phage- | Y | T | A | M | — | V | — | V | — | — | — | N | V | L | 0.0663 | 1.289 | 0.0695 | 4129 |
| 3813 | | | | | | | | | | | | | | | | | | |
| Phage- | E | F | K | W | — | V | — | D | — | — | — | Y | — | V | 0.0551 | 1.2856 | 0.0589 | 4130 |
| 3814 | | | | | | | | | | | | | | | | | | |
| Phage- | A | S | I | S | — | I | — | D | — | — | — | H | — | — | 0.0751 | 1.2813 | 0.0872 | 4131 |
| 3815 | | | | | | | | | | | | | | | | | | |
| Phage- | L | D | L | S | — | V | — | A | — | — | — | T | — | — | 0.0906 | 1.2806 | 0.0963 | 4132 |
| 3816 | | | | | | | | | | | | | | | | | | |
| Phage- | A | T | — | V | — | S | — | P | — | — | — | T | — | — | 0.0756 | 1.2733 | 0.0662 | 4133 |
| 3817 | | | | | | | | | | | | | | | | | | |
| Phage- | E | N | A | K | — | S | — | A | — | — | — | V | — | — | 0.0531 | 1.268 | 0.0752 | 4134 |
| 3818 | | | | | | | | | | | | | | | | | | |
| Phage- | V | H | I | M | — | V | — | P | — | — | — | E | R | — | 0.0567 | 1.2655 | 0.0664 | 4135 |
| 3819 | | | | | | | | | | | | | | | | | | |
| Phage- | L | L | E | R | — | V | — | S | — | — | — | L | — | — | 0.0892 | 1.2637 | 0.1022 | 4136 |
| 3820 | | | | | | | | | | | | | | | | | | |
| Phage- | P | Q | I | W | — | V | — | T | — | — | — | — | A | S | 0.0621 | 1.2601 | 0.0743 | 4137 |
| 3821 | | | | | | | | | | | | | | | | | | |
| Phage- | Q | E | P | V | — | R | — | A | — | — | — | S | — | — | 0.0743 | 1.2583 | 0.0764 | 4138 |
| 3822 | | | | | | | | | | | | | | | | | | |
| Phage- | Q | V | K | Y | — | E | — | Q | — | — | — | — | — | L | 0.0679 | 1.2537 | 0.0788 | 4139 |
| 3823 | | | | | | | | | | | | | | | | | | |
| Phage- | E | — | P | Y | — | R | — | S | — | — | — | — | — | — | 0.0512 | 1.2488 | 0.0837 | 4140 |
| 3824 | | | | | | | | | | | | | | | | | | |
| Phage- | P | G | M | V | — | K | — | S | — | — | — | S | H | L | 0.0542 | 1.2467 | 0.1068 | 4141 |
| 3825 | | | | | | | | | | | | | | | | | | |
| Phage- | V | Q | H | W | — | V | — | A | — | — | — | Y | — | L | 0.0887 | 1.2448 | 0.1069 | 4142 |
| 3826 | | | | | | | | | | | | | | | | | | |
| Phage- | V | D | R | M | — | V | — | P | — | — | — | D | V | L | 0.0624 | 1.2446 | 0.0859 | 4143 |
| 3827 | | | | | | | | | | | | | | | | | | |
| Phage- | — | D | R | H | — | I | — | A | — | — | — | E | Q | — | 0.0524 | 1.244 | 0.0571 | 4144 |
| 3828 | | | | | | | | | | | | | | | | | | |
| Phage- | P | E | I | R | — | A | — | L | — | — | — | V | — | — | 0.0567 | 1.2433 | 0.0725 | 4145 |
| 3829 | | | | | | | | | | | | | | | | | | |
| Phage- | P | P | R | A | — | V | — | S | — | — | — | T | — | — | 0.06 | 1.2433 | 0.0854 | 4146 |
| 3830 | | | | | | | | | | | | | | | | | | |
| Phage- | G | M | K | T | — | V | — | A | — | — | — | V | — | — | 0.0653 | 1.2409 | 0.08 | 4147 |
| 3831 | | | | | | | | | | | | | | | | | | |
| Phage- | T | S | — | — | — | — | — | L | — | — | — | S | — | — | 0.0877 | 1.2395 | 0.0785 | 4148 |
| 3832 | | | | | | | | | | | | | | | | | | |
| Phage- | V | G | I | R | — | E | — | A | — | — | — | E | — | — | 0.0647 | 1.235 | 0.0645 | 4149 |
| 3833 | | | | | | | | | | | | | | | | | | |
| Phage- | V | D | R | V | — | I | — | T | — | — | — | E | — | I | 0.0641 | 1.2324 | 0.0575 | 4150 |
| 3834 | | | | | | | | | | | | | | | | | | |
| Phage- | F | W | Q | R | — | V | — | T | — | — | — | E | — | — | 0.0927 | 1.2283 | 0.1482 | 4151 |
| 3835 | | | | | | | | | | | | | | | | | | |
| Phage- | V | L | H | K | — | L | — | T | — | — | — | N | — | — | 0.0842 | 1.2275 | 0.0967 | 4152 |
| 3836 | | | | | | | | | | | | | | | | | | |
| Phage- | D | P | — | W | — | I | — | Q | — | — | — | E | — | — | 0.0944 | 1.227 | 0.0652 | 4153 |
| 3837 | | | | | | | | | | | | | | | | | | |
| Phage- | S | D | Y | M | — | R | — | S | — | — | — | E | — | L | 0.1382 | 1.2251 | 0.0632 | 4154 |
| 3838 | | | | | | | | | | | | | | | | | | |
| Phage- | N | Y | N | A | — | V | — | I | — | — | — | E | — | — | 0.0825 | 1.2234 | 0.1044 | 4155 |
| 3839 | | | | | | | | | | | | | | | | | | |
| Phage- | P | Y | S | I | — | K | — | D | — | — | — | T | — | L | 0.0728 | 1.2219 | 0.0653 | 4156 |
| 3840 | | | | | | | | | | | | | | | | | | |
| Phage- | S | E | M | R | — | S | — | P | — | — | — | Y | — | — | 0.0745 | 1.2218 | 0.0822 | 4157 |
| 3841 | | | | | | | | | | | | | | | | | | |
| Phage- | L | Y | E | M | — | K | — | L | — | — | — | — | — | — | 0.0531 | 1.2203 | 0.0737 | 4158 |
| 3842 | | | | | | | | | | | | | | | | | | |
| Phage- | P | F | L | A | — | I | — | Q | — | — | — | E | V | V | 0.0579 | 1.2201 | 0.2475 | 4159 |
| 3843 | | | | | | | | | | | | | | | | | | |
| Phage- | S | D | M | V | — | K | — | P | — | — | — | Y | — | — | 0.0675 | 1.2183 | 0.0897 | 4160 |
| 3844 | | | | | | | | | | | | | | | | | | |
| Phage- | S | G | L | S | — | V | — | F | — | — | — | T | — | — | 0.0527 | 1.2134 | 0.0662 | 4161 |
| 3845 | | | | | | | | | | | | | | | | | | |
| Phage- | Y | N | Q | — | — | I | — | — | — | — | — | T | — | — | 0.0532 | 1.2131 | 0.0729 | 4162 |
| 3846 | | | | | | | | | | | | | | | | | | |
| Phage- | E | L | R | — | — | V | — | H | — | — | — | D | — | — | 0.076 | 1.2127 | 0.0777 | 4163 |
| 3847 | | | | | | | | | | | | | | | | | | |
| Phage- | V | P | R | — | — | M | — | P | — | — | — | V | — | — | 0.0773 | 1.2078 | 0.0968 | 4164 |
| 3848 | | | | | | | | | | | | | | | | | | |
| Phage- | W | D | I | R | — | S | — | E | — | — | — | I | — | L | 0.0551 | 1.2058 | 0.0603 | 4165 |
| 3849 | | | | | | | | | | | | | | | | | | |
| Phage- | E | P | K | E | — | V | — | P | — | — | — | I | — | M | 0.0704 | 1.2052 | 0.079 | 4166 |
| 3850 | | | | | | | | | | | | | | | | | | |
| Phage- | W | Q | A | W | — | E | — | P | — | — | — | L | — | — | 0.086 | 1.1992 | 0.0962 | 4167 |
| 3851 | | | | | | | | | | | | | | | | | | |
| Phage- | D | Q | R | M | — | E | — | S | — | — | — | L | — | L | 0.0549 | 1.1943 | 0.0704 | 4168 |
| 3852 | | | | | | | | | | | | | | | | | | |
| Phage- | V | V | H | — | — | T | — | — | — | — | — | N | — | — | 0.0541 | 1.1874 | 0.0576 | 4169 |
| 3853 | | | | | | | | | | | | | | | | | | |
| Phage- | V | S | K | M | — | S | — | V | — | — | — | E | — | — | 0.0662 | 1.1852 | 0.0928 | 4170 |
| 3854 | | | | | | | | | | | | | | | | | | |
| Phage- | N | Y | — | K | — | R | — | A | — | — | — | D | — | — | 0.0567 | 1.1782 | 0.0759 | 4171 |
| 3855 | | | | | | | | | | | | | | | | | | |
| Phage- | Y | E | L | A | — | V | — | R | — | — | — | Y | — | L | 0.0818 | 1.1775 | 0.0797 | 4172 |
| 3856 | | | | | | | | | | | | | | | | | | |
| Phage- | S | D | H | — | — | I | — | R | — | — | — | V | — | — | 0.0549 | 1.1751 | 0.0875 | 4173 |
| 3857 | | | | | | | | | | | | | | | | | | |
| Phage- | G | S | H | Y | — | V | — | Q | — | — | — | G | — | — | 0.0573 | 1.1721 | 0.0618 | 4174 |
| 3858 | | | | | | | | | | | | | | | | | | |
| Phage- | E | H | A | R | — | T | — | A | — | — | — | — | — | — | 0.0612 | 1.1721 | 0.0767 | 4175 |
| 3859 | | | | | | | | | | | | | | | | | | |
| Phage- | — | E | S | I | — | R | — | A | — | — | — | E | R | L | 0.0864 | 1.1717 | 0.0726 | 4176 |
| 3860 | | | | | | | | | | | | | | | | | | |
| Phage- | V | Q | I | V | — | S | — | T | — | — | — | T | — | — | 0.0531 | 1.1711 | 0.0524 | 4177 |
| 3861 | | | | | | | | | | | | | | | | | | |
| Phage- | P | S | Q | M | — | V | — | T | — | — | — | V | — | — | 0.0667 | 1.1703 | 0.0743 | 4178 |
| 3862 | | | | | | | | | | | | | | | | | | |
| Phage- | Q | L | G | A | — | I | — | W | — | — | — | N | — | — | 0.0672 | 1.1657 | 0.0752 | 4179 |
| 3863 | | | | | | | | | | | | | | | | | | |
| Phage- | N | — | G | Y | — | V | — | T | — | — | — | D | — | — | 0.0622 | 1.1652 | 0.1059 | 4180 |
| 3864 | | | | | | | | | | | | | | | | | | |
| Phage- | M | G | — | E | — | K | — | L | — | — | — | T | — | L | 0.0591 | 1.1624 | 0.0596 | 4181 |
| 3865 | | | | | | | | | | | | | | | | | | |
| Phage- | G | Q | G | S | — | V | — | M | — | — | — | E | — | — | 0.0601 | 1.1615 | 0.0673 | 4182 |
| 3866 | | | | | | | | | | | | | | | | | | |
| Phage- | L | S | W | E | — | V | — | Q | — | — | — | T | — | L | 0.0711 | 1.1605 | 0.0592 | 4183 |
| 3867 | | | | | | | | | | | | | | | | | | |
| Phage- | P | Y | T | — | — | R | — | A | — | — | — | Q | A | M | 0.0867 | 1.1586 | 0.2205 | 4184 |
| 3868 | | | | | | | | | | | | | | | | | | |
| Phage- | L | E | I | F | — | V | — | M | — | — | — | E | — | L | 0.0511 | 1.1559 | 0.0623 | 4185 |
| 3869 | | | | | | | | | | | | | | | | | | |
| Phage- | A | V | I | M | — | E | — | S | — | — | — | E | — | — | 0.0773 | 1.1555 | 0.1018 | 4186 |
| 3870 | | | | | | | | | | | | | | | | | | |
| Phage- | E | G | I | — | — | K | — | S | — | — | — | N | D | — | 0.086 | 1.1504 | 0.0803 | 4187 |
| 3871 | | | | | | | | | | | | | | | | | | |
| Phage- | S | T | M | — | — | S | — | S | — | — | — | F | — | — | 0.0623 | 1.1479 | 0.073 | 4188 |
| 3872 | | | | | | | | | | | | | | | | | | |
| Phage- | L | Q | Q | — | — | V | — | — | — | — | — | V | — | — | 0.0615 | 1.1456 | 0.0902 | 4189 |
| 3873 | | | | | | | | | | | | | | | | | | |
| Phage- | Y | D | R | — | — | S | — | P | — | — | — | S | — | — | 0.0573 | 1.1453 | 0.0629 | 4190 |
| 3874 | | | | | | | | | | | | | | | | | | |
| Phage- | P | Q | I | K | — | I | — | A | — | — | — | D | — | — | 0.0645 | 1.1425 | 0.0851 | 4191 |
| 3875 | | | | | | | | | | | | | | | | | | |
| Phage- | S | N | — | M | — | R | — | S | — | — | — | — | — | — | 0.0548 | 1.1416 | 0.1319 | 4192 |
| 3876 | | | | | | | | | | | | | | | | | | |
| Phage- | E | D | A | — | — | V | — | Q | — | — | — | S | — | — | 0.0577 | 1.1405 | 0.0648 | 4193 |
| 3877 | | | | | | | | | | | | | | | | | | |
| Phage- | T | — | P | M | — | N | — | P | — | — | — | Y | — | L | 0.0589 | 1.1344 | 0.0701 | 4194 |
| 3878 | | | | | | | | | | | | | | | | | | |
| Phage- | V | T | P | A | — | M | — | R | — | — | — | E | — | — | 0.0626 | 1.133 | 0.0611 | 4195 |
| 3879 | | | | | | | | | | | | | | | | | | |
| Phage- | — | E | K | Q | — | V | — | Q | — | — | — | D | — | — | 0.059 | 1.1292 | 0.0745 | 4196 |
| 3880 | | | | | | | | | | | | | | | | | | |
| Phage- | E | S | K | — | — | I | — | D | — | — | — | I | L | L | 0.0589 | 1.1275 | 0.0662 | 4197 |
| 3881 | | | | | | | | | | | | | | | | | | |
| Phage- | M | D | — | W | — | V | — | Q | — | — | — | D | — | — | 0.0612 | 1.1209 | 0.0645 | 4198 |
| 3882 | | | | | | | | | | | | | | | | | | |
| Phage- | V | D | Y | V | — | R | — | S | — | — | — | S | — | I | 0.0603 | 1.1206 | 0.0618 | 4199 |
| 3883 | | | | | | | | | | | | | | | | | | |
| Phage- | T | E | P | A | — | V | — | P | — | — | — | V | — | W | 0.0782 | 1.1152 | 0.1275 | 4200 |
| 3884 | | | | | | | | | | | | | | | | | | |
| Phage- | G | M | — | R | — | V | — | D | — | — | — | Y | — | — | 0.0636 | 1.1146 | 0.0613 | 4201 |
| 3885 | | | | | | | | | | | | | | | | | | |
| Phage- | T | E | H | Q | — | V | — | A | — | — | — | G | — | — | 0.0535 | 1.1106 | 0.0809 | 4202 |
| 3886 | | | | | | | | | | | | | | | | | | |
| Phage- | — | L | R | H | — | T | — | E | — | — | — | I | — | L | 0.055 | 1.1054 | 0.0689 | 4203 |
| 3887 | | | | | | | | | | | | | | | | | | |
| Phage- | L | Q | I | R | — | — | — | V | — | — | — | H | — | — | 0.0889 | 1.1045 | 0.0801 | 4204 |
| 3888 | | | | | | | | | | | | | | | | | | |
| Phage- | — | D | T | — | — | V | — | W | — | — | — | S | — | — | 0.0798 | 1.1045 | 0.0774 | 4205 |
| 3889 | | | | | | | | | | | | | | | | | | |
| Phage- | D | Q | H | A | — | R | — | E | — | — | — | V | — | — | 0.0566 | 1.1015 | 0.0789 | 4206 |
| 3890 | | | | | | | | | | | | | | | | | | |
| Phage- | V | V | Y | T | — | V | — | S | — | — | — | T | — | L | 0.0742 | 1.1003 | 0.062 | 4207 |
| 3891 | | | | | | | | | | | | | | | | | | |
| Phage- | D | Y | W | Y | — | — | — | A | — | — | — | T | — | L | 0.0551 | 1.0956 | 0.0585 | 4208 |
| 3892 | | | | | | | | | | | | | | | | | | |
| Phage- | L | V | P | R | — | L | — | E | — | — | — | T | — | — | 0.0754 | 1.0922 | 0.0848 | 4209 |
| 3893 | | | | | | | | | | | | | | | | | | |
| Phage- | D | L | Q | M | — | L | — | K | — | — | — | V | — | — | 0.0536 | 1.0895 | 0.0639 | 4210 |
| 3894 | | | | | | | | | | | | | | | | | | |
| Phage- | E | W | I | M | — | T | — | P | — | — | — | H | — | — | 0.0635 | 1.0867 | 0.0638 | 4211 |
| 3895 | | | | | | | | | | | | | | | | | | |
| Phage- | P | D | — | H | — | V | — | R | — | — | — | D | — | L | 0.0834 | 1.0842 | 0.0633 | 4212 |
| 3896 | | | | | | | | | | | | | | | | | | |
| Phage- | L | T | L | K | — | A | — | A | — | — | — | N | — | — | 0.0768 | 1.0796 | 0.0619 | 4213 |
| 3897 | | | | | | | | | | | | | | | | | | |
| Phage- | E | S | Q | Y | — | V | — | R | — | — | — | D | — | — | 0.0708 | 1.0777 | 0.0633 | 4214 |
| 3898 | | | | | | | | | | | | | | | | | | |
| Phage- | F | E | R | V | — | V | — | A | — | — | — | — | — | — | 0.059 | 1.068 | 0.0668 | 4215 |
| 3899 | | | | | | | | | | | | | | | | | | |
| Phage- | G | N | D | Y | — | V | — | A | — | — | — | — | — | — | 0.0555 | 1.0648 | 0.0974 | 4216 |
| 3900 | | | | | | | | | | | | | | | | | | |
| Phage- | Y | V | M | M | — | V | — | Q | — | — | — | T | A | K | 0.0583 | 1.0635 | 0.069 | 4217 |
| 3901 | | | | | | | | | | | | | | | | | | |
| Phage- | G | H | M | Y | — | S | — | L | — | — | — | E | — | — | 0.0628 | 1.0624 | 0.1309 | 4218 |
| 3902 | | | | | | | | | | | | | | | | | | |
| Phage- | L | G | P | M | — | R | — | — | — | — | — | V | — | L | 0.0674 | 1.0604 | 0.0797 | 4219 |
| 3903 | | | | | | | | | | | | | | | | | | |
| Phage- | G | Y | H | — | — | I | — | E | — | — | — | S | — | L | 0.0635 | 1.0595 | 0.0923 | 4220 |
| 3904 | | | | | | | | | | | | | | | | | | |
| Phage- | A | D | — | — | — | R | — | H | — | — | — | — | — | — | 0.052 | 1.0586 | 0.0581 | 4221 |
| 3905 | | | | | | | | | | | | | | | | | | |
| Phage- | S | D | A | — | — | R | — | H | — | — | — | — | — | — | 0.0531 | 1.0584 | 0.061 | 4222 |
| 3906 | | | | | | | | | | | | | | | | | | |
| Phage- | S | L | E | R | — | E | — | P | — | — | — | F | — | — | 0.0696 | 1.0583 | 0.0697 | 4223 |
| 3907 | | | | | | | | | | | | | | | | | | |
| Phage- | T | E | R | M | — | S | — | S | — | — | — | D | — | — | 0.0595 | 1.0517 | 0.0693 | 4224 |
| 3908 | | | | | | | | | | | | | | | | | | |
| Phage- | V | M | A | V | — | — | — | E | — | — | — | S | — | M | 0.0734 | 1.0506 | 0.0731 | 4225 |
| 3909 | | | | | | | | | | | | | | | | | | |
| Phage- | V | I | P | — | — | R | — | S | — | — | — | — | A | Y | 0.0822 | 1.0393 | 0.1161 | 4226 |
| 3910 | | | | | | | | | | | | | | | | | | |
| Phage- | E | S | R | A | — | V | — | — | — | — | — | H | — | L | 0.0573 | 1.0316 | 0.06 | 4227 |
| 3911 | | | | | | | | | | | | | | | | | | |
| Phage- | A | T | — | R | — | M | — | M | — | — | — | H | — | — | 0.0635 | 1.0314 | 0.0901 | 4228 |
| 3912 | | | | | | | | | | | | | | | | | | |
| Phage- | Q | N | F | H | — | V | — | Q | — | — | — | D | W | L | 0.0644 | 1.0188 | 0.0986 | 4229 |
| 3913 | | | | | | | | | | | | | | | | | | |
| Phage- | F | G | — | K | — | E | — | R | — | — | — | N | — | — | 0.0807 | 1.0174 | 0.0693 | 4230 |
| 3914 | | | | | | | | | | | | | | | | | | |
| Phage- | Y | Q | M | — | — | V | — | E | — | — | — | S | — | L | 0.0517 | 1.0163 | 0.0874 | 4231 |
| 3915 | | | | | | | | | | | | | | | | | | |
| Phage- | S | T | T | — | — | R | — | A | — | — | — | T | — | M | 0.0588 | 1.0155 | 0.0655 | 4232 |
| 3916 | | | | | | | | | | | | | | | | | | |
| Phage- | D | R | — | — | — | T | — | T | — | — | — | E | — | — | 0.0623 | 1.0145 | 0.0642 | 4233 |
| 3917 | | | | | | | | | | | | | | | | | | |
| Phage- | A | Q | K | — | — | E | — | M | — | — | — | V | — | — | 0.0661 | 1.0045 | 0.064 | 4234 |
| 3918 | | | | | | | | | | | | | | | | | | |
| Phage- | M | S | P | M | — | S | — | S | — | — | — | M | — | — | 0.0566 | 0.9934 | 0.0911 | 4235 |
| 3919 | | | | | | | | | | | | | | | | | | |
| Phage- | A | Q | — | — | — | V | — | — | — | — | — | T | — | — | 0.061 | 0.9851 | 0.0618 | 4236 |
| 3920 | | | | | | | | | | | | | | | | | | |
| Phage- | G | Q | I | M | — | I | — | T | — | — | — | S | — | — | 0.0695 | 0.9803 | 0.0809 | 4237 |
| 3921 | | | | | | | | | | | | | | | | | | |
| Phage- | G | — | Y | T | — | V | — | A | — | — | — | Q | — | — | 0.0645 | 0.979 | 0.0665 | 4238 |
| 3922 | | | | | | | | | | | | | | | | | | |
| Phage- | — | E | A | Q | — | R | — | V | — | — | — | H | — | — | 0.0677 | 0.9779 | 0.0779 | 4239 |
| 3923 | | | | | | | | | | | | | | | | | | |
| Phage- | Y | N | Q | Y | — | T | — | L | — | — | — | E | — | — | 0.0713 | 0.9769 | 0.0796 | 4240 |
| 3924 | | | | | | | | | | | | | | | | | | |
| Phage- | N | E | R | F | — | E | — | A | — | — | — | H | — | — | 0.064 | 0.9765 | 0.0675 | 4241 |
| 3925 | | | | | | | | | | | | | | | | | | |
| Phage- | V | E | R | F | — | E | — | A | — | — | — | I | — | L | 0.0737 | 0.9721 | 0.1621 | 4242 |
| 3926 | | | | | | | | | | | | | | | | | | |
| Phage- | S | M | Q | K | — | V | — | S | — | — | — | — | — | — | 0.0615 | 0.9719 | 0.0588 | 4243 |
| 3927 | | | | | | | | | | | | | | | | | | |
| Phage- | W | D | L | — | — | S | — | P | — | — | — | — | — | M | 0.0928 | 0.9706 | 0.0733 | 4244 |
| 3928 | | | | | | | | | | | | | | | | | | |
| Phage- | T | W | S | I | — | E | — | R | — | — | — | E | — | — | 0.0547 | 0.9704 | 0.0777 | 4245 |
| 3929 | | | | | | | | | | | | | | | | | | |
| Phage- | Q | L | L | R | — | V | — | P | — | — | — | E | — | — | 0.0852 | 0.9696 | 0.1055 | 4246 |
| 3930 | | | | | | | | | | | | | | | | | | |
| Phage- | P | T | I | M | — | I | — | V | — | — | — | Q | A | — | 0.0734 | 0.9655 | 0.0695 | 4247 |
| 3931 | | | | | | | | | | | | | | | | | | |
| Phage- | L | N | — | E | — | V | — | Q | — | — | — | D | — | — | 0.0596 | 0.9654 | 0.1093 | 4248 |
| 3932 | | | | | | | | | | | | | | | | | | |
| Phage- | Y | T | Q | A | — | T | — | S | — | — | — | H | — | — | 0.0591 | 0.9595 | 0.0861 | 4249 |
| 3933 | | | | | | | | | | | | | | | | | | |
| Phage- | D | I | — | E | — | R | — | R | — | — | — | D | — | — | 0.058 | 0.9363 | 0.0593 | 4250 |
| 3934 | | | | | | | | | | | | | | | | | | |
| Phage- | P | G | F | W | — | — | — | K | — | — | — | E | — | — | 0.0582 | 0.9301 | 0.0652 | 4251 |
| 3935 | | | | | | | | | | | | | | | | | | |
| Phage- | W | V | R | — | — | — | — | A | — | — | — | T | — | L | 0.0728 | 0.926 | 0.1415 | 4252 |
| 3936 | | | | | | | | | | | | | | | | | | |
| Phage- | L | T | P | V | — | R | — | A | — | — | — | L | E | L | 0.0613 | 0.9223 | 0.0566 | 4253 |
| 3937 | | | | | | | | | | | | | | | | | | |
| Phage- | M | Q | D | F | — | K | — | L | — | — | — | S | — | — | 0.0552 | 0.9211 | 0.0585 | 4254 |
| 3938 | | | | | | | | | | | | | | | | | | |
| Phage- | Y | — | R | E | — | I | — | Q | — | — | — | G | — | — | 0.0529 | 0.9178 | 0.0765 | 4255 |
| 3939 | | | | | | | | | | | | | | | | | | |
| Phage- | P | L | T | S | — | V | — | Q | — | — | — | Y | — | — | 0.0555 | 0.9131 | 0.0873 | 4256 |
| 3940 | | | | | | | | | | | | | | | | | | |
| Phage- | L | V | R | M | — | — | — | P | — | — | — | D | — | — | 0.0569 | 0.9125 | 0.0699 | 4257 |
| 3941 | | | | | | | | | | | | | | | | | | |
| Phage- | G | D | I | Q | — | R | — | L | — | — | — | D | — | L | 0.0876 | 0.9069 | 0.1277 | 4258 |
| 3942 | | | | | | | | | | | | | | | | | | |
| Phage- | D | Q | — | M | — | I | — | R | — | — | — | E | — | — | 0.0559 | 0.9043 | 0.0676 | 4259 |
| 3943 | | | | | | | | | | | | | | | | | | |
| Phage- | E | F | R | E | — | R | — | P | — | — | — | Q | — | L | 0.0773 | 0.8986 | 0.0789 | 4260 |
| 3944 | | | | | | | | | | | | | | | | | | |
| Phage- | D | S | L | V | — | — | — | R | — | — | — | V | — | — | 0.0588 | 0.895 | 0.063 | 4261 |
| 3945 | | | | | | | | | | | | | | | | | | |
| Phage- | S | T | M | S | — | V | — | S | — | — | — | L | — | — | 0.0663 | 0.8851 | 0.0614 | 4262 |
| 3946 | | | | | | | | | | | | | | | | | | |
| Phage- | T | Q | — | K | — | I | — | L | — | — | — | D | — | — | 0.0734 | 0.8849 | 0.0544 | 4263 |
| 3947 | | | | | | | | | | | | | | | | | | |
| Phage- | L | T | — | Y | — | T | — | E | — | — | — | S | — | W | 0.0734 | 0.882 | 0.0749 | 4264 |
| 3948 | | | | | | | | | | | | | | | | | | |
| Phage- | G | W | — | G | — | V | — | L | — | — | — | E | — | — | 0.066 | 0.8779 | 0.0698 | 4265 |
| 3949 | | | | | | | | | | | | | | | | | | |
| Phage- | — | L | P | R | — | E | — | Y | — | — | — | T | — | L | 0.077 | 0.8723 | 0.0634 | 4266 |
| 3950 | | | | | | | | | | | | | | | | | | |
| Phage- | T | V | R | S | — | T | — | P | — | — | — | E | — | — | 0.0752 | 0.8713 | 0.0856 | 4267 |
| 3951 | | | | | | | | | | | | | | | | | | |
| Phage- | A | V | H | M | — | A | — | S | — | — | — | S | — | — | 0.0893 | 0.87 | 0.0825 | 4268 |
| 3952 | | | | | | | | | | | | | | | | | | |
| Phage- | G | L | P | Q | — | V | — | P | — | — | — | D | — | — | 0.0554 | 0.8636 | 0.056 | 4269 |
| 3953 | | | | | | | | | | | | | | | | | | |
| Phage- | F | F | Q | V | — | E | — | Q | — | — | — | L | — | — | 0.0578 | 0.8539 | 0.0548 | 4270 |
| 3954 | | | | | | | | | | | | | | | | | | |
| Phage- | Y | E | R | V | — | V | — | S | — | — | — | N | S | L | 0.0645 | 0.8377 | 0.0845 | 4271 |
| 3955 | | | | | | | | | | | | | | | | | | |
| Phage- | D | V | L | M | — | K | — | E | — | — | — | L | — | Y | 0.0539 | 0.8367 | 0.0628 | 4272 |
| 3956 | | | | | | | | | | | | | | | | | | |
| Phage- | T | I | Q | W | — | E | — | L | — | — | — | R | — | — | 0.0575 | 0.8353 | 0.0643 | 4273 |
| 3957 | | | | | | | | | | | | | | | | | | |
| Phage- | T | — | P | W | — | — | — | R | — | — | — | D | — | — | 0.0686 | 0.8276 | 0.0701 | 4274 |
| 3958 | | | | | | | | | | | | | | | | | | |
| Phage- | A | E | M | — | — | V | — | P | — | — | — | K | A | — | 0.0545 | 0.8225 | 0.0615 | 4275 |
| 3959 | | | | | | | | | | | | | | | | | | |
| Phage- | E | G | G | Y | — | V | — | T | — | — | — | S | — | — | 0.0806 | 0.8217 | 0.0786 | 4276 |
| 3960 | | | | | | | | | | | | | | | | | | |
| Phage- | L | V | S | W | — | T | — | P | — | — | — | E | — | V | 0.0655 | 0.8184 | 0.1314 | 4277 |
| 3961 | | | | | | | | | | | | | | | | | | |
| Phage- | F | D | G | W | — | V | — | A | — | — | — | Y | P | — | 0.0758 | 0.8158 | 0.0796 | 4278 |
| 3962 | | | | | | | | | | | | | | | | | | |
| Phage- | — | S | A | E | — | R | — | P | — | — | — | I | — | — | 0.0543 | 0.8147 | 0.0626 | 4279 |
| 3963 | | | | | | | | | | | | | | | | | | |
| Phage- | — | T | R | M | — | T | — | S | — | — | — | M | S | — | 0.0616 | 0.8144 | 0.078 | 4280 |
| 3964 | | | | | | | | | | | | | | | | | | |
| Phage- | M | — | T | M | — | V | — | Q | — | — | — | — | — | — | 0.0675 | 0.8135 | 0.1412 | 4281 |
| 3965 | | | | | | | | | | | | | | | | | | |
| Phage- | G | S | — | F | — | I | — | T | — | — | — | E | Q | S | 0.0547 | 0.8128 | 0.07 | 4282 |
| 3966 | | | | | | | | | | | | | | | | | | |
| Phage- | G | G | — | M | — | R | — | M | — | — | — | — | — | — | 0.0794 | 0.8105 | 0.0836 | 4283 |
| 3967 | | | | | | | | | | | | | | | | | | |
| Phage- | E | I | G | A | — | V | — | D | — | — | — | R | — | — | 0.072 | 0.8095 | 0.0694 | 4284 |
| 3968 | | | | | | | | | | | | | | | | | | |
| Phage- | S | T | L | — | — | T | — | E | — | — | — | F | — | M | 0.0573 | 0.7893 | 0.0553 | 4285 |
| 3969 | | | | | | | | | | | | | | | | | | |
| Phage- | S | Y | M | R | — | E | — | E | — | — | — | S | — | L | 0.086 | 0.7858 | 0.1215 | 4286 |
| 3970 | | | | | | | | | | | | | | | | | | |
| Phage- | V | Q | T | R | — | V | — | A | — | — | — | D | — | — | 0.0633 | 0.7819 | 0.0744 | 4287 |
| 3971 | | | | | | | | | | | | | | | | | | |
| Phage- | D | — | R | — | — | T | — | L | — | — | — | T | — | — | 0.0621 | 0.7808 | 0.0622 | 4288 |
| 3972 | | | | | | | | | | | | | | | | | | |
| Phage- | Q | M | S | M | — | K | — | A | — | — | — | T | — | L | 0.0629 | 0.7672 | 0.0909 | 4289 |
| 3973 | | | | | | | | | | | | | | | | | | |
| Phage- | Q | Q | — | R | — | V | — | D | — | — | — | S | — | — | 0.0617 | 0.762 | 0.0755 | 4290 |
| 3974 | | | | | | | | | | | | | | | | | | |
| Phage- | E | — | K | W | — | T | — | Q | — | — | — | G | — | — | 0.0656 | 0.7558 | 0.0703 | 4291 |
| 3975 | | | | | | | | | | | | | | | | | | |
| Phage- | P | — | Q | I | — | M | — | A | — | — | — | H | — | — | 0.0915 | 0.7538 | 0.0646 | 4292 |
| 3976 | | | | | | | | | | | | | | | | | | |
| Phage- | D | — | R | M | — | I | — | M | — | — | — | Q | A | — | 0.067 | 0.7423 | 0.0713 | 4293 |
| 3977 | | | | | | | | | | | | | | | | | | |
| Phage- | G | F | R | T | — | V | — | S | — | — | — | T | — | — | 0.0797 | 0.7336 | 0.0906 | 4294 |
| 3978 | | | | | | | | | | | | | | | | | | |
| Phage- | L | S | — | E | — | I | — | V | — | — | — | N | — | — | 0.0672 | 0.7322 | 0.0832 | 4295 |
| 3979 | | | | | | | | | | | | | | | | | | |
| Phage- | E | Y | R | E | — | V | — | D | — | — | — | S | — | — | 0.0519 | 0.7311 | 0.0722 | 4296 |
| 3980 | | | | | | | | | | | | | | | | | | |
| Phage- | V | V | — | Q | — | I | — | E | — | — | — | N | — | — | 0.0852 | 0.7286 | 0.0683 | 4297 |
| 3981 | | | | | | | | | | | | | | | | | | |
| Phage- | S | Y | Q | — | — | K | — | E | — | — | — | H | — | W | 0.0643 | 0.7147 | 0.0692 | 4298 |
| 3982 | | | | | | | | | | | | | | | | | | |
| Phage- | Y | E | R | M | — | S | — | E | — | — | — | W | D | — | 0.0628 | 0.7044 | 0.0861 | 4299 |
| 3983 | | | | | | | | | | | | | | | | | | |
| Phage- | K | H | I | — | — | E | — | V | — | — | — | V | — | — | 0.065 | 0.6972 | 0.0708 | 4300 |
| 3984 | | | | | | | | | | | | | | | | | | |
| Phage- | V | E | N | — | — | R | — | S | — | — | — | E | — | — | 0.0687 | 0.696 | 0.112 | 4301 |
| 3985 | | | | | | | | | | | | | | | | | | |
| Phage- | N | G | P | M | — | V | — | Q | — | — | — | D | — | T | 0.0593 | 0.6914 | 0.0558 | 4302 |
| 3986 | | | | | | | | | | | | | | | | | | |
| Phage- | H | V | Q | R | — | V | — | Q | — | — | — | V | — | — | 0.0832 | 0.6856 | 0.1194 | 4303 |
| 3987 | | | | | | | | | | | | | | | | | | |
| Phage- | S | T | P | Y | — | V | — | D | — | — | — | — | — | — | 0.0965 | 0.683 | 0.0729 | 4304 |
| 3988 | | | | | | | | | | | | | | | | | | |
| Phage- | M | T | D | R | — | K | — | E | — | — | — | F | — | — | 0.0772 | 0.6766 | 0.1418 | 4305 |
| 3989 | | | | | | | | | | | | | | | | | | |
| Phage- | L | L | I | V | — | K | — | P | — | — | — | — | — | S | 0.0912 | 0.6665 | 0.0992 | 4306 |
| 3990 | | | | | | | | | | | | | | | | | | |
| Phage- | S | V | M | N | — | V | — | L | R | — | — | E | — | — | 0.069 | 0.6444 | 0.0968 | 4307 |
| 3991 | | | | | | | | | | | | | | | | | | |
| Phage- | V | F | K | M | — | V | — | M | — | — | — | Q | V | S | 0.0673 | 0.6352 | 0.0687 | 4308 |
| 3992 | | | | | | | | | | | | | | | | | | |
| Phage- | — | — | — | K | — | M | — | A | — | — | — | V | — | I | 0.0646 | 0.6318 | 0.0897 | 4309 |
| 3993 | | | | | | | | | | | | | | | | | | |
| Phage- | E | W | P | I | — | R | — | S | — | — | — | D | D | — | 0.0719 | 0.6303 | 0.0604 | 4310 |
| 3994 | | | | | | | | | | | | | | | | | | |
| Phage- | M | D | — | W | — | V | — | T | — | — | — | Q | H | L | 0.0597 | 0.6271 | 0.1193 | 4311 |
| 3995 | | | | | | | | | | | | | | | | | | |
| Phage- | G | T | Q | A | — | V | — | L | — | — | — | E | — | I | 0.0656 | 0.6201 | 0.0787 | 4312 |
| 3996 | | | | | | | | | | | | | | | | | | |
| Phage- | V | Q | P | W | — | V | — | A | — | — | — | D | — | L | 0.088 | 0.6068 | 0.0983 | 4313 |
| 3997 | | | | | | | | | | | | | | | | | | |
| Phage- | Q | M | I | T | — | — | — | K | — | — | — | E | — | — | 0.0544 | 0.5887 | 0.0852 | 4314 |
| 3998 | | | | | | | | | | | | | | | | | | |
| Phage- | V | Q | R | V | — | V | — | T | — | — | — | D | V | — | 0.0789 | 0.5844 | 0.07 | 4315 |
| 3999 | | | | | | | | | | | | | | | | | | |
| Phage- | Q | E | M | T | — | V | — | R | — | — | — | S | — | — | 0.0827 | 0.5655 | 0.0832 | 4316 |
| 4000 | | | | | | | | | | | | | | | | | | |
| Phage- | D | G | L | I | — | — | — | S | — | — | — | T | — | L | 0.0664 | 0.5543 | 0.0705 | 4317 |
| 4001 | | | | | | | | | | | | | | | | | | |
| Phage- | M | T | N | M | — | R | — | S | — | — | — | — | V | — | 0.076 | 0.5512 | 0.1081 | 4318 |
| 4002 | | | | | | | | | | | | | | | | | | |
| Phage- | D | I | E | V | — | R | — | H | — | — | — | E | — | — | 0.0707 | 0.5421 | 0.0801 | 4319 |
| 4003 | | | | | | | | | | | | | | | | | | |
| Phage- | E | V | K | K | — | V | — | A | — | — | — | E | S | Q | 0.0496 | 0.542 | 0.0538 | 4320 |
| 4004 | | | | | | | | | | | | | | | | | | |
| Phage- | L | T | S | W | — | V | — | Q | — | — | — | D | — | L | 0.0612 | 0.523 | 0.0623 | 4321 |
| 4005 | | | | | | | | | | | | | | | | | | |
| Phage- | H | V | T | M | — | S | — | — | — | — | — | W | — | L | 0.0626 | 0.4847 | 0.0703 | 4322 |
| 4006 | | | | | | | | | | | | | | | | | | |
| Phage- | P | T | S | F | — | R | — | P | — | — | — | E | — | L | 0.0543 | 0.4749 | 0.0592 | 4323 |
| 4007 | | | | | | | | | | | | | | | | | | |
| Phage- | — | M | Q | R | — | E | — | L | — | — | — | S | — | L | 0.0574 | 0.4712 | 0.0777 | 4324 |
| 4008 | | | | | | | | | | | | | | | | | | |
| Phage- | G | P | K | — | — | L | — | M | — | — | — | Q | — | — | 0.0711 | 0.4591 | 0.0611 | 4325 |
| 4009 | | | | | | | | | | | | | | | | | | |
| Phage- | F | E | — | R | — | V | — | T | — | — | — | — | — | Q | 0.1306 | 0.4273 | 0.069 | 4326 |
| 4010 | | | | | | | | | | | | | | | | | | |
| Phage- | G | W | R | Q | — | E | — | V | — | — | — | S | — | — | 0.0669 | 0.4231 | 0.0672 | 4327 |
| 4011 | | | | | | | | | | | | | | | | | | |
| Phage- | M | R | S | V | — | V | — | S | — | — | — | S | — | — | 0.075 | 0.4217 | 0.1764 | 4328 |
| 4012 | | | | | | | | | | | | | | | | | | |
| Phage- | A | P | R | M | — | S | — | P | — | — | — | Y | E | L | 0.0714 | 0.4163 | 0.0661 | 4329 |
| 4013 | | | | | | | | | | | | | | | | | | |

(—) indicates the same amino acid position as in Peptide-180 corresponding position

L₁ and L₂

In some embodiments, L₁ is bound to N-terminus of A₁. In some embodiments, L₁ is bound to C-terminus of A₁. In some embodiments, L₂ is bound to N-terminus of A₂. In some embodiments, L₂ is bound to C-terminus of A₂. In some embodiments, L₁ or L₂ is a peptide sequence having at least 5 to no more than 50 amino acids. In some embodiments, L₁ or L₂ is a peptide sequence having at least 10 to no more than 30 amino acids. In some embodiments, L₁ or L₂ is a peptide sequence having at least 10 amino acids. In some embodiments, L₁ or L₂ is a peptide sequence having at least 18 amino acids. In some embodiments, L₁ or L₂ is a peptide sequence having at least 26 amino acids. In some embodiments. L₁ or L₂ has a formula comprising (G₂S)ₙ, wherein n is an integer from 1 to 3 (SEQ ID NO: 605). In some embodiments, L₁ has a formula selected from the group consisting of (G₂S)ₙ, (GS)ₙ, (GSGGS),(SEQ ID NO: 606), (GGGS),(SEQ ID NO: 607).(GGGGS)ₙ(SEQ ID NO: 608), and (GSSGGS),(SEQ ID NO: 609), wherein n is an integer of at least 1.

In some embodiments, P, becomes unbound from A, when L₁ is cleaved by the tumor specific protease thereby exposing A₁ to CD28. In some embodiments, P₂ becomes unbound from A₂ when L₂ is cleaved by the tumor specific protease thereby exposing A₂ to PSMA. In some embodiments, the tumor specific protease is selected from the group consisting of a matrix metalloprotease (MMP), serine protease, cysteine protease, threonine protease, and aspartic protease. In some embodiments, the matrix metalloprotease comprises MMP2, MMP7, MMP9, MMP13, or MMP14. In some embodiments, the serine protease comprises matriptase (MTSP1), urokinase, or hepsin. In some embodiments, L₁ or L₂ comprises a urokinase cleavable amino acid sequence, a matriptase cleavable amino acid sequence, matrix metalloprotease cleavable amino acid sequence, or a legumain cleavable amino acid sequence.

In some embodiments, L₁ or L₂ comprises an amino acid sequence according to SEQ ID NO: 110. In some embodiments, L₁ or L₂ comprises an amino acid sequence according to any one of SEQ ID NOs: 107-137 and 604. In some embodiments. L₁ or L₂ comprises an amino acid sequence of Linker 25 (ISSGLLSGRSDAG) (SEQ ID NO: 132), Linker 26 (AAGLLAPPGGLSGRSDAG) (SEQ ID NO: 133), Linker 27 (SPLGLSGRSDAG) (SEQ ID NO: 134), or Linker 28 (LSGRSDAGSPLGLAG) (SEQ ID NO: 135), or an amino acid sequence that has 1, 2, or 3 amino acid substitutions, additions, or deletions relative to the amino acid sequence of Linker 25, Linker 26, Linker 27, or Linker 28.

TABLE 11
L1, L2, L3, and Lia Sequences

| Construct | Amino Acid Sequence | SEQ ID |
|---|---|---|
| Description | (N to C) | NO: |
| Linker 1 | GGGGSGGGGSGGGGS | 107 |
| Linker 2 | GGGGS | 108 |
| Linker 3 | GGGGSGGGS | 109 |
| Cleavable | GGGGSGGGLSGRSDAGSPLGLAGSGGGS | 110 |
| linker | | |
| Linker 4 | GGGGSLSGRSDNHGSSGT | 111 |
| Linker 5 | GGGGSSGGSGGSGLSGRSDNHGSSGT | 112 |
| Linker 6 | ASGRSDNH | 113 |
| Linker 7 | LAGRSDNH | 114 |
| Linker 8 | ISSGLASGRSDNH | 115 |
| Linker 9 | ISSGLLAGRSDNH | 116 |
| Linker 10 | LSGRSDNH | 117 |
| Linker 11 | ISSGLLSGRSDNP | 118 |
| Linker 12 | ISSGLLSGRSDNH | 119 |
| Linker 13 | LSGRSDNHSPLGLAGS | 120 |
| Linker 14 | SPLGLAGSLSGRSDNH | 121 |
| Linker 15 | SPLGLSGRSDNH | 122 |
| Linker 16 | LAGRSDNHSPLGLAGS | 123 |
| Linker 17 | LSGRSDNHVPLSLKMG | 124 |
| Linker 18 | LSGRSDNHVPLSLSMG | 125 |
| Linker 19 | GSSGGSGGSGGSGISSGLLSGRSDNHGSSGT | 126 |
| Linker 20 | GSSGGSGGSGGISSGLLSGRSDNHGGGS | 127 |
| Linker 21 | ASGRSDNH | 128 |
| Linker 22 | LAGRSDNH | 129 |
| Linker 23 | ISSGLASGRSDNH | 130 |
| Linker 24 | LSGRSDAG | 131 |
| Linker 25 | ISSGLLSGRSDAG | 132 |
| Linker 26 | AAGLLAPPGGLSGRSDAG | 133 |
| Linker 27 | SPLGLSGRSDAG | 134 |
| Linker 28 | LSGRSDAGSPLGLAG | 135 |
| Linker 29 | LSGRSDAGSPLGLAGS | 136 |
| Non- | GGGGSGGGSGGGGSGGASSGAGGSGGGS | 137 |
| cleavable | | |
| linker | | |
| Non- | SGGGGSGGASSGAGGS | 604 |
| cleavable | | |
| linker | | |

### H₁

In some embodiments, H₁ comprises a polymer. In some embodiments, the polymer is polyethylene glycol (PEG). In some embodiments, H₁ comprises albumin. In some embodiments, H₁ comprises an Fc domain. In some embodiments, the albumin is serum albumin. In some embodiments, the albumin is human serum albumin. In some embodiments, H₁ comprises a polypeptide, a ligand, or a small molecule. In some embodiments, the polypeptide, the ligand or the small molecule binds serum protein or a fragment thereof, a circulating immunoglobulin or a fragment thereof, or CD/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, sIgA, IgM or IgD. In some embodiments, the serum protein is albumin. In some embodiments, the polypeptide is an antibody. In some embodiments, the antibody comprises a single domain antibody, a single chain variable fragment, or a Fab. In some embodiments, the single domain antibody comprises a single domain antibody that binds to albumin. In some embodiments, the single domain antibody is a human or humanized antibody. In some embodiments, the single domain antibody is 645gH1gL1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7 h-11-15. In some embodiments, the single domain antibody is DOM7 h-11-15. In some embodiments, the single domain antibody is Alb-1, Alb-8, or Alb-23. In some embodiments, the single domain antibody is 10E.

In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 142, HC-CDR2: SEQ ID NO: 143, and HC-CDR3: SEQ ID NO: 144. In some embodiments, the single domain antibody comprises an amino acid sequence with at least 80%, 85%, 90%, 95%, 99%, or 100% identity to SEQ ID NO: 141. In some embodiments, the single domain antibody comprises the amino acid sequence of SEQ ID NO: 141. In some embodiments, the single domain antibody comprises an amino acid sequence with at least 80%, 85%, 90%, 95%, 99%, or 100% identity to SEQ ID NO: 146. In some embodiments, the single domain antibody comprises the amino acid sequence of SEQ ID NO: 146. In some embodiments, the single domain antibody comprises an amino acid sequence with at least 80%, 85%, 90%, 95%, 99%, or 100% identity to SEQ ID NO: 145. In some embodiments, wherein the single domain antibody comprises an amino acid sequence with at least 80%, 85%, 90%, 95%, 99%, or 100% identity to SEQ ID NO: 4448.

TABLE 12
H₁ or H₁ₐ Sequences

| Construct Description | Amino Acid Sequence | SEQ ID NO: |
|---|---|---|
| Anti-Albumin: CDR-H1 | GSTFYTAV | 138 |
| Anti-Albumin: CDR-H2 | IRWTALTT | 139 |
| Anti-Albumin: CDR-H3 | AARGTLGLFTTADSYDY | 140 |
| Anti-albumin (HE-1) | EVQLVESGGGLVQPGGSLRLSCAASGSTFYTAV | 141 |
| | MGWVRQAPGKGLEWVAAIRWTALTTSYADSVKG | |
| | RFTISRDGAKTTLYLQMNSLRPEDTAVYYCAAR | |
| | GTLGLFTTADSYDYWGQGTLVTVSS | |
| Anti-Albumin: CDR-H1 | GFTFSKFG | 142 |
| Anti-Albumin: CDR-H2 | ISGSGRDT | 143 |
| Anti-Albumin: CDR-H3 | TIGGSLSV | 144 |
| Anti-albumin (HE-3) | EVQLVESGGGLVQPGNSLRLSCAASGFTFSKFG | 145 |
| | MSWVRQAPGKGLEWVSSISGSGRDTLYADSVKG | |
| | RFTISRDNAKTTLYLQMNSLRPEDTAVYYCTIG | |
| | GSLSVSSQGTLVTVSS | |
| Anti-albumin (HE-5) | EVQLVESGGGLVQPGGSLRLSCAASGSTFYTAV | 146 |
| | MGWVRQAPGKGLEWVAAIRWTALTTSYADSVKG | |
| | RFTISRDNAKTTLYLQMNSLRPEDTAVYYCAAR | |
| | GTLGLFTTADAYDYWGQGTLVTVSS | |
| Anti-Albumin (HE-4) | EVQLVESGGGLVQPGNSLRLSCAASGFTFSSFG | 4448 |
| | MSWVRQAPGKGLEWVSSISGSGSDTLYADSVKG | |
| | RFTISRDNAKTTLYLQMNSLRPEDTAVYYCTIG | |
| | GSLSRSSQGTLVTVSS | |

In some embodiments, the single domain antibody is SA21. In some embodiments, the polypeptide or polypeptide complex comprises a modified amino acid, a non-natural amino acid, a modified non-natural amino acid, or a combination thereof. In some embodiments, the modified amino acid or modified non-natural amino acid comprises a post-translational modification.

### L₃

In some embodiments, H₁ comprises a linking moiety (L₃) that connects H₁ to P₁. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS)ₙ(SEQ ID NO: 606), (GGGS)ₙ(SEQ ID NO: 607).(GGGGS)ₙ(SEQ ID NO: 608), and (GSSGGS)ₙ(SEQ ID NO: 609). wherein n is an integer of at least 1. In some embodiments. L₃ comprises an amino acid sequence according to GGGGSGGGSGG (SEQ ID NO: 610).

Tumor Activated Antibodies that Bind to CD28 and PSMA

In some embodiments, the polypeptide or polypeptide complex comprises amino acid sequences with at least 85% sequence identity to SEQ ID NOs: 147 and 148. In some embodiments, the polypeptide or polypeptide complex comprises amino acid sequences with at least 90% sequence identity to SEQ ID NOs: 147 and 148. In some embodiments, the polypeptide or polypeptide complex comprises amino acid sequences with at least 95% sequence identity to SEQ ID NOs: 147 and 148. In some embodiments, the polypeptide or polypeptide complex comprises amino acid sequences with at least 98% sequence identity to SEQ ID NOs: 147 and 148. In some embodiments, the polypeptide or polypeptide complex comprises the amino acid sequences of SEQ ID NOs: 147 and 148.

In some embodiments, the polypeptide or polypeptide complex comprises amino acid sequences with at least 85% sequence identity to SEQ ID NOs: 149 and 150. In some embodiments, the polypeptide or polypeptide complex comprises amino acid sequences with at least 90% sequence identity to SEQ ID NOs: 149 and 150. In some embodiments, the polypeptide or polypeptide complex comprises amino acid sequences with at least 95% sequence identity to SEQ ID NOs: 149 and 150. In some embodiments, the polypeptide or polypeptide complex comprises amino acid sequences with at least 98% sequence identity to SEQ ID NOs: 149 and 150. In some embodiments, the polypeptide or polypeptide complex comprises the amino acid sequences of SEQ ID NOs: 149 and 150.

In some embodiments, the polypeptide or polypeptide complex comprises amino acid sequences with at least 85% sequence identity to SEQ ID NOs: 151 and 152. In some embodiments, the polypeptide or polypeptide complex comprises amino acid sequences with at least 90% sequence identity to SEQ ID NOs: 151 and 152. In some embodiments, the polypeptide or polypeptide complex comprises amino acid sequences with at least 95% sequence identity to SEQ ID NOs: 151 and 152. In some embodiments, the polypeptide or polypeptide complex comprises amino acid sequences with at least 98% sequence identity to SEQ ID NOs: 151 and 152. In some embodiments, the polypeptide or polypeptide complex comprises the amino acid sequences of SEQ ID NOs: 151 and 152.

In some embodiments, the polypeptide or polypeptide complex comprises amino acid sequences with at least 85% sequence identity to SEQ ID NOs: 153 and 154. In some embodiments, the polypeptide or polypeptide complex comprises amino acid sequences with at least 90% sequence identity to SEQ ID NOs: 153 and 154. In some embodiments, the polypeptide or polypeptide complex comprises amino acid sequences with at least 95% sequence identity to SEQ ID NOs: 153 and 154. In some embodiments, the polypeptide or polypeptide complex comprises amino acid sequences with at least 98% sequence identity to SEQ ID NOs: 153 and 154. In some embodiments, the polypeptide or polypeptide complex comprises the amino acid sequences of SEQ ID NOs: 153 and 154.

In some embodiments, the polypeptide or polypeptide complex comprises amino acid sequences with at least 85% sequence identity to SEQ ID NOs: 155 and 156. In some embodiments, the polypeptide or polypeptide complex comprises amino acid sequences with at least 90% sequence identity to SEQ ID NOs: 155 and 156. In some embodiments, the polypeptide or polypeptide complex comprises amino acid sequences with at least 95% sequence identity to SEQ ID NOs: 155 and 156. In some embodiments, the polypeptide or polypeptide complex comprises amino acid sequences with at least 98% sequence identity to SEQ ID NOs: 155 and 156. In some embodiments, the polypeptide or polypeptide complex comprises the amino acid sequences of SEQ ID NOs: 155 and 156.

In some embodiments, the polypeptide or polypeptide complex comprises amino acid sequences with at least 85% sequence identity to SEQ ID NOs: 157 and 158. In some embodiments, the polypeptide or polypeptide complex comprises amino acid sequences with at least 90% sequence identity to SEQ ID NOs: 157 and 158. In some embodiments, the polypeptide or polypeptide complex comprises amino acid sequences with at least 95% sequence identity to SEQ ID NOs: 157 and 158. In some embodiments, the polypeptide or polypeptide complex comprises amino acid sequences with at least 98% sequence identity to SEQ ID NOs: 157 and 158. In some embodiments, the polypeptide or polypeptide complex comprises the amino acid sequences of SEQ ID NOs: 157 and 158.

In some embodiments, the polypeptide or polypeptide complex comprises amino acid sequences with at least 85% sequence identity to SEQ ID NOs: 159 and 160. In some embodiments, the polypeptide or polypeptide complex comprises amino acid sequences with at least 90% sequence identity to SEQ ID NOs: 159 and 160. In some embodiments, the polypeptide or polypeptide complex comprises amino acid sequences with at least 95% sequence identity to SEQ ID NOs: 159 and 160. In some embodiments, the polypeptide or polypeptide complex comprises amino acid sequences with at least 98% sequence identity to SEQ ID NOs: 159 and 160. In some embodiments, the polypeptide or polypeptide complex comprises the amino acid sequences of SEQ ID NOs: 159 and 160.

In some embodiments, the polypeptide or polypeptide complex comprises amino acid sequences with at least 85% sequence identity to SEQ ID NOs: 161 and 162. In some embodiments, the polypeptide or polypeptide complex comprises amino acid sequences with at least 90% sequence identity to SEQ ID NOs: 161 and 162. In some embodiments, the polypeptide or polypeptide complex comprises amino acid sequences with at least 95% sequence identity to SEQ ID NOs: 161 and 162. In some embodiments, the polypeptide or polypeptide complex comprises amino acid sequences with at least 98% sequence identity to SEQ ID NOs: 161 and 162. In some embodiments, the polypeptide or polypeptide complex comprises the amino acid sequences of SEQ ID NOs: 161 and 162.

In some embodiments, the polypeptide or polypeptide complex comprises amino acid sequences with at least 85% sequence identity to SEQ ID NOs: 163 and 164. In some embodiments, the polypeptide or polypeptide complex comprises amino acid sequences with at least 90% sequence identity to SEQ ID NOs: 163 and 164. In some embodiments, the polypeptide or polypeptide complex comprises amino acid sequences with at least 95% sequence identity to SEQ ID NOs: 163 and 164. In some embodiments, the polypeptide or polypeptide complex comprises amino acid sequences with at least 98% sequence identity to SEQ ID NOs: 163 and 164. In some embodiments, the polypeptide or polypeptide complex comprises the amino acid sequences of SEQ ID NOs: 163 and 164.

In some embodiments, the polypeptide or polypeptide complex comprises amino acid sequences with at least 85% sequence identity to SEQ ID NOs: 165 and 166. In some embodiments, the polypeptide or polypeptide complex comprises amino acid sequences with at least 90% sequence identity to SEQ ID NOs: 165 and 166. In some embodiments, the polypeptide or polypeptide complex comprises amino acid sequences with at least 95% sequence identity to SEQ ID NOs: 165 and 166. In some embodiments, the polypeptide or polypeptide complex comprises amino acid sequences with at least 98% sequence identity to SEQ ID NOs: 165 and 166. In some embodiments, the polypeptide or polypeptide complex comprises the amino acid sequences of SEQ ID NOs: 165 and 166.

In some embodiments, the polypeptide or polypeptide complex comprises amino acid sequences with at least 85% sequence identity to SEQ ID NOs: 167 and 168. In some embodiments, the polypeptide or polypeptide complex comprises amino acid sequences with at least 90% sequence identity to SEQ ID NOs: 167 and 168. In some embodiments, the polypeptide or polypeptide complex comprises amino acid sequences with at least 95% sequence identity to SEQ ID NOs: 167 and 168. In some embodiments, the polypeptide or polypeptide complex comprises amino acid sequences with at least 98% sequence identity to SEQ ID NOs: 167 and 168. In some embodiments, the polypeptide or polypeptide complex comprises the amino acid sequences of SEQ ID NOs: 167 and 168.

In some embodiments, the polypeptide or polypeptide complex comprises amino acid sequences with at least 85% sequence identity to SEQ ID NOs: 169 and 170. In some embodiments, the polypeptide or polypeptide complex comprises amino acid sequences with at least 90% sequence identity to SEQ ID NOs: 169 and 170. In some embodiments, the polypeptide or polypeptide complex comprises amino acid sequences with at least 95% sequence identity to SEQ ID NOs: 169 and 170. In some embodiments, the polypeptide or polypeptide complex comprises amino acid sequences with at least 98% sequence identity to SEQ ID NOs: 169 and 170. In some embodiments, the polypeptide or polypeptide complex comprises the amino acid sequences of SEQ ID NOs: 169 and 170.

In some embodiments, the polypeptide or polypeptide complex comprises amino acid sequences with at least 85% sequence identity to SEQ ID NOs: 171 and 172. In some embodiments, the polypeptide or polypeptide complex comprises amino acid sequences with at least 90% sequence identity to SEQ ID NOs: 171 and 172. In some embodiments, the polypeptide or polypeptide complex comprises amino acid sequences with at least 95% sequence identity to SEQ ID NOs: 171 and 172. In some embodiments, the polypeptide or polypeptide complex comprises amino acid sequences with at least 98% sequence identity to SEQ ID NOs: 171 and 172. In some embodiments, the polypeptide or polypeptide complex comprises the amino acid sequences of SEQ ID NOs: 171 and 172.

In some embodiments, the polypeptide or polypeptide complex comprises amino acid sequences with at least 85% sequence identity to SEQ ID NOs: 173 and 174. In some embodiments, the polypeptide or polypeptide complex comprises amino acid sequences with at least 90% sequence identity to SEQ ID NOs: 173 and 174. In some embodiments, the polypeptide or polypeptide complex comprises amino acid sequences with at least 95% sequence identity to SEQ ID NOs: 173 and 174. In some embodiments, the polypeptide or polypeptide complex comprises amino acid sequences with at least 98% sequence identity to SEQ ID NOs: 173 and 174. In some embodiments, the polypeptide or polypeptide complex comprises the amino acid sequences of SEQ ID NOs: 173 and 174.

In some embodiments, the polypeptide or polypeptide complex comprises amino acid sequences with at least 85% sequence identity to SEQ ID NOs: 175 and 176. In some embodiments, the polypeptide or polypeptide complex comprises amino acid sequences with at least 90% sequence identity to SEQ ID NOs: 175 and 176. In some embodiments, the polypeptide or polypeptide complex comprises amino acid sequences with at least 95% sequence identity to SEQ ID NOs: 175 and 176. In some embodiments, the polypeptide or polypeptide complex comprises amino acid sequences with at least 98% sequence identity to SEQ ID NOs: 175 and 176. In some embodiments, the polypeptide or polypeptide complex comprises the amino acid sequences of SEQ ID NOs: 175 and 176.

In some embodiments, the polypeptide or polypeptide complex comprises amino acid sequences with at least 85% sequence identity to SEQ ID NOs: 177 and 178. In some embodiments, the polypeptide or polypeptide complex comprises amino acid sequences with at least 90% sequence identity to SEQ ID NOs: 177 and 178. In some embodiments, the polypeptide or polypeptide complex comprises amino acid sequences with at least 95% sequence identity to SEQ ID NOs: 177 and 178. In some embodiments, the polypeptide or polypeptide complex comprises amino acid sequences with at least 98% sequence identity to SEQ ID NOs: 177 and 178. In some embodiments, the polypeptide or polypeptide complex comprises the amino acid sequences of SEQ ID NOs: 177 and 178.

In some embodiments, the polypeptide or polypeptide complex comprises amino acid sequences with at least 80%, 85%, 90%, 95%, 99%, or 100% sequence identity to SEQ ID NO: 4330 and 4331. In some embodiments, the polypeptide or polypeptide complex comprises amino acid sequences with at least 80%, 85/, 90%, 95%, 99%, or 100% sequence identity to SEQ ID NO: 4332 and 4333. In some embodiments, the polypeptide or polypeptide complex comprises amino acid sequences with at least 80%, 85%, 90%, 95%, 99%, or 100% sequence identity to SEQ ID NO: 4334 and 4335. In some embodiments, the polypeptide or polypeptide complex comprises amino acid sequences with at least 80%, 85%, 90%, 95%, 99%, or 1000/a sequence identity to SEQ ID NO: 4336 and 4337.

In some embodiments, the polypeptide or polypeptide complex comprises amino acid sequences with at least 80%, 85%, 90%, 95%, 99%, or 100% sequence identity to SEQ ID NO: 4338 and 4339. In some embodiments, the polypeptide or polypeptide complex comprises amino acid sequences with at least 80%, 85%, 90%, 95%, 99%, or 100% sequence identity to SEQ ID NO: 4340 and 4341. In some embodiments, the polypeptide or polypeptide complex comprises amino acid sequences with at least 80%, 85%, 90%, 95%, 99%, or 100% sequence identity to SEQ ID NO: 4342 and 4343. In some embodiments, the polypeptide or polypeptide complex comprises amino acid sequences with at least 80%, 85%, 90%, 95%, 99%, or 100% sequence identity to SEQ ID NO: 4344 and 4345. In some embodiments, the polypeptide or polypeptide complex comprises amino acid sequences with at least 80%, 85%, 90%, 95%, 99%, or 100% sequence identity to SEQ ID NO: 4346 and 4347. In some embodiments, the polypeptide or polypeptide complex comprises amino acid sequences with at least 80%, 85%, 90%, 95%, 99%, or 100% sequence identity to SEQ ID NO: 4348 and 4349. In some embodiments, the polypeptide or polypeptide complex comprises amino acid sequences with at least 80%, 85%, 90%, 95%, 99%, or 100% sequence identity to SEQ ID NO: 4350 and 4351. In some embodiments, the polypeptide or polypeptide complex comprises amino acid sequences with at least 80%, 85%, 90%, 95%, 99%, or 100% sequence identity to SEQ ID NO: 4352 and 4353. In some embodiments, the polypeptide or polypeptide complex comprises amino acid sequences with at least 80%, 85%, 90%, 95%, 99%, or 100% sequence identity to SEQ ID NO: 4352 and 4353. In some embodiments, the polypeptide or polypeptide complex comprises amino acid sequences with at least 80%, 85%, 90%, 95%, 99%, or 100% sequence identity to SEQ ID NO: 4354 and 4355. In some embodiments, the polypeptide or polypeptide complex comprises amino acid sequences with at least 80%, 85%, 90%, 95%, 99%, or 100% sequence identity to SEQ ID NO: 4356 and 4357. In some embodiments, the polypeptide or polypeptide complex comprises amino acid sequences with at least 80%, 85%, 90%, 95%, 99%, or 100% sequence identity to SEQ ID NO: 4358 and 4359. In some embodiments, the polypeptide or polypeptide complex comprises amino acid sequences with at least 80%, 85%, 90%, 95%, 99%, or 100% sequence identity to SEQ ID NO: 4360 and 4361. In some embodiments, the polypeptide or polypeptide complex comprises amino acid sequences with at least 80%, 85%, 90%, 95%, 99%, or 100% sequence identity to SEQ ID NO: 4362 and 4363. In some embodiments, the polypeptide or polypeptide complex comprises amino acid sequences with at least 80%, 85%, 90%, 95%, 99%, or 100% sequence identity to SEQ ID NO: 4364 and 4365. In some embodiments, the polypeptide or polypeptide complex comprises amino acid sequences with at least 80%, 85%, 90%, 95%, 99%, or 100% sequence identity to SEQ ID NO: 4366 and 4367. In some embodiments, the polypeptide or polypeptide complex comprises amino acid sequences with at least 80%, 85%, 90%, 95%, 99%, or 100% sequence identity to SEQ ID NO: 4368 and 4369. In some embodiments, the polypeptide or polypeptide complex comprises amino acid sequences with at least 80%, 85%, 90%, 95%, 99%, or 100% sequence identity to SEQ ID NO: 4370 and 4371. In some embodiments, the polypeptide or polypeptide complex comprises amino acid sequences with at least 80%, 85%, 90%, 95%, 99%, or 100% sequence identity to SEQ ID NO: 4372 and 4373. In some embodiments, the polypeptide or polypeptide complex comprises amino acid sequences with at least 80%, 85%, 90%, 95%, 99%, or 100% sequence identity to SEQ ID NO: 4374 and 4375. In some embodiments, the polypeptide or polypeptide complex comprises amino acid sequences with at least 80%, 85%, 90%, 95%, 99%, or 100% sequence identity to SEQ ID NO: 4376 and 4377. In some embodiments, the polypeptide or polypeptide complex comprises amino acid sequences with at least 80%, 85%, 90%, 95%, 99%, or 100% sequence identity to SEQ ID NO: 4378 and 4379. In some embodiments, the polypeptide or polypeptide complex comprises amino acid sequences with at least 80%, 85%, 90%, 95%, 99%, or 100% sequence identity to SEQ ID NO: 4380 and 4381. In some embodiments, the polypeptide or polypeptide complex comprises amino acid sequences with at least 80%, 85%, 90%, 95%, 99%, or 100% sequence identity to SEQ ID NO: 4382 and 4383. In some embodiments, the polypeptide or polypeptide complex comprises amino acid sequences with at least 80%, 85%, 90%, 95%, 99%, or 100% sequence identity to SEQ ID NO: 4384 and 4385. In some embodiments, the polypeptide or polypeptide complex comprises amino acid sequences with at least 80%, 85%, 90%, 95%, 99%, or 100% sequence identity to SEQ ID NO: 4386 and 4387.

In some embodiments, the polypeptide or polypeptide complex comprises amino acid sequences with at least 80%, 85%, 90%, 95%, 99%, or 100% sequence identity to SEQ ID NO: 4386 and 4387. In some embodiments, the polypeptide or polypeptide complex comprises amino acid sequences with at least 80%, 85%, 90%, 95%, 99%, or 100% sequence identity to SEQ ID NO: 4388 and 4389. In some embodiments, the polypeptide or polypeptide complex comprises amino acid sequences with at least 80%, 85%, 90%, 95%, 99%, or 100% sequence identity to SEQ ID NO: 4390 and 4391. In some embodiments, the polypeptide or polypeptide complex comprises amino acid sequences with at least 80%, 85%, 90%, 95%, 99%, or 100% sequence identity to SEQ ID NO: 4392 and 4393. In some embodiments, the polypeptide or polypeptide complex comprises amino acid sequences with at least 80%, 85%, 90%, 95%, 99%, or 100% sequence identity to SEQ ID NO: 4394 and 4395. In some embodiments, the polypeptide or polypeptide complex comprises amino acid sequences with at least 80%, 85%, 90%, 95%, 99%, or 100% sequence identity to SEQ ID NO: 4394 and 4395. In some embodiments, the polypeptide or polypeptide complex comprises amino acid sequences with at least 80%, 85%, 90%, 95%, 99%, or 100% sequence identity to SEQ ID NO: 4396 and 4397. In some embodiments, the polypeptide or polypeptide complex comprises amino acid sequences with at least 80%, 85%, 90%, 95%, 99%, or 100% sequence identity to SEQ ID NO: 4398 and 4399. In some embodiments, the polypeptide or polypeptide complex comprises amino acid sequences with at least 80%, 85%, 90%, 95%, 99%, or 100% sequence identity to SEQ ID NO: 4400 and 4401. In some embodiments, the polypeptide or polypeptide complex comprises amino acid sequences with at least 80%, 85%, 90%, 95%, 99%, or 100% sequence identity to SEQ ID NO: 4402 and 4403. In some embodiments, the polypeptide or polypeptide complex comprises amino acid sequences with at least 80%, 85%, 90/, 95%, 99%, or 100% sequence identity to SEQ ID NO: 4404 and 4405. In some embodiments, the polypeptide or polypeptide complex comprises amino acid sequences with at least 80%, 85%, 90%, 95%, 99%, or 100% sequence identity to SEQ ID NO: 4406 and 4407. In some embodiments, the polypeptide or polypeptide complex comprises amino acid sequences with at least 80%, 85%, 90%, 95%, 99%, or 100% sequence identity to SEQ ID NO: 4408 and 4409. In some embodiments, the polypeptide or polypeptide complex comprises amino acid sequences with at least 80%, 85%, 90%, 95%, 99%, or 100% sequence identity to SEQ ID NO: 4410 and 4411. In some embodiments, the polypeptide or polypeptide complex comprises amino acid sequences with at least 80%, 85%, 90%, 95%, 99%, or 100% sequence identity to SEQ ID NO: 4412 and 4413. In some embodiments, the polypeptide or polypeptide complex comprises amino acid sequences with at least 80%, 85%, 90%, 95%, 99%, or 100% sequence identity to SEQ ID NO: 4414 and 4415. In some embodiments, the polypeptide or polypeptide complex comprises amino acid sequences with at least 80%, 85%, 90%, 95%, 99%, or 100% sequence identity to SEQ ID NO: 4416 and 4417. In some embodiments, the polypeptide or polypeptide complex comprises amino acid sequences with at least 80%, 85%, 90%, 95%, 99%, or 100% sequence identity to SEQ ID NO: 4418 and 4419. In some embodiments, the polypeptide or polypeptide complex comprises amino acid sequences with at least 80%, 85%, 90%, 95%, 99%, or 100% sequence identity to SEQ ID NO: 4420 and 4421. In some embodiments, the polypeptide or polypeptide complex comprises amino acid sequences with at least 80%, 85%, 90%, 95%, 99%, or 100% sequence identity to SEQ ID NO: 4422 and 4423. In some embodiments, the polypeptide or polypeptide complex comprises amino acid sequences with at least 80%, 85%, 90%, 95%, 99%, or 100% sequence identity to SEQ ID NO: 4424 and 4425. In some embodiments, the polypeptide or polypeptide complex comprises amino acid sequences with at least 80%, 85%, 90%, 95%, 99%, or 100% sequence identity to SEQ ID NO: 4426 and 4427. In some embodiments, the polypeptide or polypeptide complex comprises amino acid sequences with at least 80%, 85%, 90%, 95%, 99%, or 100% sequence identity to SEQ ID NO: 4428 and 4429. In some embodiments, the polypeptide or polypeptide complex comprises amino acid sequences with at least 80%, 85%, 90%, 95%, 99%, or 100% sequence identity to SEQ ID NO: 4430 and 4431. In some embodiments, the polypeptide or polypeptide complex comprises amino acid sequences with at least 80%, 85%, 90%, 95%, 99%, or 100% sequence identity to SEQ ID NO: 4432 and 4433. In some embodiments, the polypeptide or polypeptide complex comprises amino acid sequences with at least 80%, 85%, 90%, 95%, 99%, or 100% sequence identity to SEQ ID NO: 4434 and 4435. In some embodiments, the polypeptide or polypeptide complex comprises amino acid sequences with at least 80%, 85%, 90%, 95%, 99%, or 100% sequence identity to SEQ ID NO: 4436 and 4437. In some embodiments, the polypeptide or polypeptide complex comprises amino acid sequences with at least 80%, 85%, 90%, 95%, 99%, or 100% sequence identity to SEQ ID NO: 4438 and 4439. In some embodiments, the polypeptide or polypeptide complex comprises amino acid sequences with at least 80%, 85%, 90%, 95%, 99%, or 100% sequence identity to SEQ ID NO: 4440 and 4441. In some embodiments, the polypeptide or polypeptide complex comprises amino acid sequences with at least 80%, 85%, 90%, 95%, 99%, or 100% sequence identity to SEQ ID NO: 4442 and 4443. In some embodiments, the polypeptide or polypeptide complex comprises amino acid sequences with at least 80%, 85%, 90%, 95%, 99%, or 100% sequence identity to SEQ ID NO: 4444 and 4445. In some embodiments, the polypeptide or polypeptide complex comprises amino acid sequences with at least 80%, 85%, 90%, 95%, 99%, or 100% sequence identity to SEQ ID NO: 4446 and 4447.

TABLE 13
Tumor Activated Antibodies that Bind to CD28 and PSMA

| | Amino Acid Sequence | SEQ ID |
|---|---|---|
| Construct Description | (N to C) | NO: |
| PC-1 LC | DIQMTQSPSSLSASVGDRVTITCRASQGISNYLAW | 147 |
| albumin binder: HE-5 | YQQKTGKVPKFLIYEASTLQSGVPSRFSGGGSGTD | |
| CD28 mask: Peptide-41 | FTLTISSLQPEDVATYYCQNYNSAPFTFGPGTKVD | |
| linker: Linker 29 | IKRTVAAPSVFIFPPSDEQLKSGTASVVCLLNNFYP | |
| CD28 scFv: wt Vh-liker-Vl | REAKVQWKVDNALQSGNSQESVTEQDSKDSTYS | |
| | LSSTLTLSKADYEKHKVYACEVTHQGLSSPVTKS | |
| | FNRGEC | |
| PC-1 HC | EVQLVESGGGLVQPGGSLRLSCAASGSTFYTAVM | 148 |
| albumin binder: HE-5 | GWVRQAPGKGLEWVAAIRWTALTTSYADSVKG | |
| CD28 mask: Peptide-41 | RFTISRDNAKTTLYLQMNSLRPEDTAVYYCAARG | |
| linker: Linker 29 | TLGLFTTADAYDYWGQGTLVTVSSGGGGSGGGS | |
| CD28 scFv: wt Vh-liker-Vl | GGMDWCPRDLWVHCFAGSSGGSGGLSGRSDAGS | |
| | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSQVQLVESGGGVV | |
| | QPGRSLRLSCAASGFAFSRYGMHWVRQAPGKGL | |
| | EWVAVIWYDGSNKYYADSVKGRFTISRDNSKNT | |
| | QYLQMNSLRAEDTAVYYCARGGDFLYYYYYGM | |
| | DVWGQGTTVTVSSASTKGPSVFPLAPSSKSTSGGT | |
| | AALGCLVKDYFPEPVTVSWNSGALTSGVHTFPAV | |
| | LQSSGLYSLSSVVTVPSSSLGTQTYICNVNHKPSN | |
| | TKVDKKVEPKSC | |
| PC-2 LC | EVQLVESGGGLVQPGGSLRLSCAASGSTFYTAVM | 149 |
| albumin binder: HE-5 | GWVRQAPGKGLEWVAAIRWTALTTSYADSVKG | |
| CD28 mask: Peptide-41 | RFTISRDNAKTTLYLQMNSLRPEDTAVYYCAARG | |
| linker: Linker 29 | TLGLFTTADAYDYWGQGTLVTVSSGGGGSGGGS | |
| CD28 scFv: wt Vh-linker-Vl | GGMDWCPRDLWVHCFAGSSGGSGGLSGRSDAGS | |
| | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-2 HC | QVQLVESGGGVVQPGRSLRLSCAASGFAFSRYGM | 150 |
| albumin binder: HE-5 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: Linker 29 | DFLYYYYYGMDVWGQGTTVTVSSASTKGPSVFP | |
| CD28 scFv: wt Vh-linker-Vl | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-3 LC | EVQLVESGGGLVQPGGSLRLSCAASGSTFYTAVM | 151 |
| albumin binder: HE-5 | GWVRQAPGKGLEWVAAIRWTALTTSYADSVKG | |
| CD28 mask: Peptide-41 | RFTISRDNAKTTLYLQMNSLRPEDTAVYYCAARG | |
| linker: Linker 29 | TLGLFTTADAYDYWGQGTLVTVSSGGGGSGGGS | |
| CD28 scFv: disulfide Vh-linker- | GGMDWCPRDLWVHCFAGSSGGSGGLSGRSDAGS | |
| Vl | PLGLAGSGGGSQVQLVQSGAEVKKPGASVKVSC | |
| | KASGYTFTSYYIHWVRQAPGQCLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGCGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-3 HC | QVQLVESGGGVVQPGRSLRLSCAASGFAFSRYGM | 152 |
| albumin binder: HE-5 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: Linker 29 | DFLYYYYYGMDVWGQGTTVTVSSASTKGPSVFP | |
| CD28 scFv: disulfide Vh-linker- | LAPSSKSTSGGTAALGCLVKDYFPEPVTVSWNSG | |
| Vl | ALTSGVHTFPAVLQSSGLYSLSSVVTVPSSSLGTQ | |
| | TYICNVNHKPSNTKVDKKVEPKSC | |
| PC-4 LC | EVQLVESGGGLVQPGGSLRLSCAASGSTFYTAVM | 153 |
| albumin binder: HE-5 | GWVRQAPGKGLEWVAAIRWTALTTSYADSVKG | |
| CD28 mask: Peptide-41 | RFTISRDNAKTTLYLQMNSLRPEDTAVYYCAARG | |
| linker: non-cleavable | TLGLFTTADAYDYWGQGTLVTVSSGGGGSGGGS | |
| CD28 scFv: disulfide Vh-linker- | GGMDWCPRDLWVHCFAGGGGSGGGSGGGGSGG | |
| Vl | ASSGAGGSGGGSQVQLVQSGAEVKKPGASVKVS | |
| | CKASGYTFTSYYIHWVRQAPGQCLEWIGSIYPGN | |
| | VNTNYNEKFKDRATLTVDTSISTAYMELSRLRSD | |
| | DTAVYFCTRSHYGLDWNFDVWGQGTTVTVSSGG | |
| | GGSGGGGSGGGGSDIQMTQSPSSLSASVGDRVTIT | |
| | CHASQNIYVWLNWYQQKPGKAPKLLIYKASNLH | |
| | TGVPSRFSGSGSGTDFTLTISSLQPEDFATYYCQQ | |
| | GQTYPYTFGCGTKVEIKGGGGSDIQMTQSPSSLSA | |
| | SVGDRVTITCRASQGISNYLAWYQQKTGKVPKFL | |
| | IYEASTLQSGVPSRFSGGGSGTDFTLTISSLQPEDV | |
| | ATYYCQNYNSAPFTFGPGTKVDIKRTVAAPSVFIF | |
| | PPSDEQLKSGTASVVCLLNNFYPREAKVQWKVD | |
| | NALQSGNSQESVTEQDSKDSTYSLSSTLTLSKADY | |
| | EKHKVYACEVTHQGLSSPVTKSFNRGEC | |
| PC-4 HC | QVQLVESGGGVVQPGRSLRLSCAASGFAFSRYGM | 154 |
| albumin binder: HE-5 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: non-cleavable | DFLYYYYYGMDVWGQGTTVTVSSASTKGPSVFP | |
| CD28 scFv: disulfide Vh-linker- | LAPSSKSTSGGTAALGCLVKDYFPEPVTVSWNSG | |
| Vl | ALTSGVHTFPAVLQSSGLYSLSSVVTVPSSSLGTQ | |
| | TYICNVNHKPSNTKVDKKVEPKSC | |
| PC-5 LC | EVQLVESGGGLVQPGGSLRLSCAASGSTFYTAVM | 155 |
| albumin binder: HE-1 | GWVRQAPGKGLEWVAAIRWTALTTSYADSVKG | |
| CD28 mask: Peptide-41 | RFTISRDGAKTTLYLQMNSLRPEDTAVYYCAARG | |
| linker: Linker 29 | TLGLFTTADSYDYWGQGTLVTVSSGGGGSGGGS | |
| CD28 scFv: disulfide Vh-linker- | GGMDWCPRDLWVHCFAGSSGGSGGLSGRSDAGS | |
| Vl | PLGLAGSGGGSQVQLVQSGAEVKKPGASVKVSC | |
| | KASGYTFTSYYIHWVRQAPGQCLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGCGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-5 HC | QVQLVESGGGVVQPGRSLRLSCAASGFAFSRYGM | 156 |
| albumin binder: HE-1 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: Linker 29 | DFLYYYYYGMDVWGQGTTVTVSSASTKGPSVFP | |
| CD28 scFv: disulfide Vh-linker- | LAPSSKSTSGGTAALGCLVKDYFPEPVTVSWNSG | |
| Vl | ALTSGVHTFPAVLQSSGLYSLSSVVTVPSSSLGTQ | |
| | TYICNVNHKPSNTKVDKKVEPKSC | |
| PC-6 LC | EVQLVESGGGLVQPGGSLRLSCAASGSTFYTAVM | 157 |
| albumin binder: HE-1 | GWVRQAPGKGLEWVAAIRWTALTTSYADSVKG | |
| CD28 mask: Peptide-41 | RFTISRDGAKTTLYLQMNSLRPEDTAVYYCAARG | |
| linker: non-cleavable | TLGLFTTADSYDYWGQGTLVTVSSGGGGSGGGS | |
| CD28 scFv: disulfide Vh-linker- | GGMDWCPRDLWVHCFAGGGGSGGGSGGGGSGG | |
| Vl | ASSGAGGSGGGSQVQLVQSGAEVKKPGASVKVS | |
| | CKASGYTFTSYYIHWVRQAPGQCLEWIGSIYPGN | |
| | VNTNYNEKFKDRATLTVDTSISTAYMELSRLRSD | |
| | DTAVYFCTRSHYGLDWNFDVWGQGTTVTVSSGG | |
| | GGSGGGGSGGGGSDIQMTQSPSSLSASVGDRVTIT | |
| | CHASQNIYVWLNWYQQKPGKAPKLLIYKASNLH | |
| | TGVPSRFSGSGSGTDFTLTISSLQPEDFATYYCQQ | |
| | GQTYPYTFGCGTKVEIKGGGGSDIQMTQSPSSLSA | |
| | SVGDRVTITCRASQGISNYLAWYQQKTGKVPKFL | |
| | IYEASTLQSGVPSRFSGGGSGTDFTLTISSLQPEDV | |
| | ATYYCQNYNSAPFTFGPGTKVDIKRTVAAPSVFIF | |
| | PPSDEQLKSGTASVVCLLNNFYPREAKVQWKVD | |
| | NALQSGNSQESVTEQDSKDSTYSLSSTLTLSKADY | |
| | EKHKVYACEVTHQGLSSPVTKSFNRGEC | |
| PC-6 HC | QVQLVESGGGVVQPGRSLRLSCAASGFAFSRYGM | 158 |
| albumin binder: HE-1 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: non-cleavable | DFLYYYYYGMDVWGQGTTVTVSSASTKGPSVFP | |
| CD28 scFv: disulfide Vh-linker- | LAPSSKSTSGGTAALGCLVKDYFPEPVTVSWNSG | |
| Vl | ALTSGVHTFPAVLQSSGLYSLSSVVTVPSSSLGTQ | |
| | TYICNVNHKPSNTKVDKKVEPKSC | |
| PC-7 LC | EVQLVESGGGLVQPGGSLRLSCAASGSTFYTAVM | 159 |
| albumin binder: HE-1 | GWVRQAPGKGLEWVAAIRWTALTTSYADSVKG | |
| CD28 mask: Peptide-41 | RFTISRDGAKTTLYLQMNSLRPEDTAVYYCAARG | |
| linker: Linker 29 | TLGLFTTADSYDYWGQGTLVTVSSGGGGSGGGS | |
| CD28 scFv: wt Vh-linker-Vl | GGMDWCPRDLWVHCFAGSSGGSGGLSGRSDAGS | |
| | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-7 HC | QVQLVESGGGVVQPGRSLRLSCAASGFAFSRYGM | 160 |
| albumin binder: HE-1 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: Linker 29 | DFLYYYYYGMDVWGQGTTVTVSSASTKGPSVFP | |
| CD28 scFv: wt Vh-linker-Vl | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-8 LC | EVQLVESGGGLVQPGGSLRLSCAASGSTFYTAVM | 161 |
| albumin binder: HE-1 | GWVRQAPGKGLEWVAAIRWTALTTSYADSVKG | |
| CD28 mask: Peptide-41 | RFTISRDGAKTTLYLQMNSLRPEDTAVYYCAARG | |
| linker: non-cleavable | TLGLFTTADSYDYWGQGTLVTVSSGGGGSGGGS | |
| CD28 scFv: wt Vh-linker-Vl | GGMDWCPRDLWVHCFAGGGGSGGGSGGGGSGG | |
| | ASSGAGGSGGGSQVQLVQSGAEVKKPGASVKVS | |
| | CKASGYTFTSYYIHWVRQAPGQGLEWIGSIYPGN | |
| | VNTNYNEKFKDRATLTVDTSISTAYMELSRLRSD | |
| | DTAVYFCTRSHYGLDWNFDVWGQGTTVTVSSGG | |
| | GGSGGGGSGGGGSDIQMTQSPSSLSASVGDRVTIT | |
| | CHASQNIYVWLNWYQQKPGKAPKLLIYKASNLH | |
| | TGVPSRFSGSGSGTDFTLTISSLQPEDFATYYCQQ | |
| | GQTYPYTFGGGTKVEIKGGGGSDIQMTQSPSSLSA | |
| | SVGDRVTITCRASQGISNYLAWYQQKTGKVPKFL | |
| | IYEASTLQSGVPSRFSGGGSGTDFTLTISSLQPEDV | |
| | ATYYCQNYNSAPFTFGPGTKVDIKRTVAAPSVFIF | |
| | PPSDEQLKSGTASVVCLLNNFYPREAKVQWKVD | |
| | NALQSGNSQESVTEQDSKDSTYSLSSTLTLSKADY | |
| | EKHKVYACEVTHQGLSSPVTKSFNRGEC | |
| PC-8 HC | QVQLVESGGGVVQPGRSLRLSCAASGFAFSRYGM | 162 |
| albumin binder: HE-1 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: non-cleavable | DFLYYYYYGMDVWGQGTTVTVSSASTKGPSVFP | |
| CD28 scFv: wt Vh-linker-Vl | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-9 LC | EVQLVESGGGLVQPGGSLRLSCAASGSTFYTAVM | 163 |
| albumin binder: HE-1 | GWVRQAPGKGLEWVAAIRWTALTTSYADSVKG | |
| CD28 mask: Peptide-41 | RFTISRDGAKTTLYLQMNSLRPEDTAVYYCAARG | |
| linker: Linker 29 | TLGLFTTADSYDYWGQGTLVTVSSGGGGSGGGS | |
| CD28 scFv: wt Vl-linker-Vh | GGMDWCPRDLWVHCFAGSSGGSGGLSGRSDAGS | |
| | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-9 HC | QVQLVESGGGVVQPGRSLRLSCAASGFAFSRYGM | 164 |
| albumin binder: HE-1 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: Linker 29 | DFLYYYYYGMDVWGQGTTVTVSSASTKGPSVFP | |
| CD28 scFv: wt Vl-linker-Vh | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-10 LC | EVQLVESGGGLVQPGGSLRLSCAASGSTFYTAVM | 165 |
| albumin binder: HE-1 | GWVRQAPGKGLEWVAAIRWTALTTSYADSVKG | |
| CD28 mask: Peptide-41 | RFTISRDGAKTTLYLQMNSLRPEDTAVYYCAARG | |
| linker: Linker 29 | TLGLFTTADSYDYWGQGTLVTVSSGGGGSGGGS | |
| CD28 scFv: disulfide Vl-linker- | GGMDWCPRDLWVHCFAGSSGGSGGLSGRSDAGS | |
| Vh | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGCGTKVEIKGGGGSGGGGSGGGGSQVQL | |
| | VQSGAEVKKPGASVKVSCKASGYTFTSYYIHWV | |
| | RQAPGQCLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-10 HC | QVQLVESGGGVVQPGRSLRLSCAASGFAFSRYGM | 166 |
| albumin binder: HE-1 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: Linker 29 | DFLYYYYYGMDVWGQGTTVTVSSASTKGPSVFP | |
| CD28 scFv: disulfide Vl-linker- | LAPSSKSTSGGTAALGCLVKDYFPEPVTVSWNSG | |
| Vh | ALTSGVHTFPAVLQSSGLYSLSSVVTVPSSSLGTQ | |
| | TYICNVNHKPSNTKVDKKVEPKSC | |
| PC-11 LC | EVQLVESGGGLVQPGGSLRLSCAASGSTFYTAVM | 167 |
| albumin binder: HE-1 | GWVRQAPGKGLEWVAAIRWTALTTSYADSVKG | |
| CD28 mask: Peptide-41 | RFTISRDGAKTTLYLQMNSLRPEDTAVYYCAARG | |
| linker: non-cleavable | TLGLFTTADSYDYWGQGTLVTVSSGGGGSGGGS | |
| CD28 scFv: wt Vl-linker-Vh | GGMDWCPRDLWVHCFAGGGGSGGGSGGGGSGG | |
| | ASSGAGGSGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSGGGGSGGGGSQV | |
| | QLVQSGAEVKKPGASVKVSCKASGYTFTSYYIHW | |
| | VRQAPGQGLEWIGSIYPGNVNTNYNEKFKDRATL | |
| | TVDTSISTAYMELSRLRSDDTAVYFCTRSHYGLD | |
| | WNFDVWGQGTTVTVSSGGGGSDIQMTQSPSSLSA | |
| | SVGDRVTITCRASQGISNYLAWYQQKTGKVPKFL | |
| | IYEASTLQSGVPSRFSGGGSGTDFTLTISSLQPEDV | |
| | ATYYCQNYNSAPFTFGPGTKVDIKRTVAAPSVFIF | |
| | PPSDEQLKSGTASVVCLLNNFYPREAKVQWKVD | |
| | NALQSGNSQESVTEQDSKDSTYSLSSTLTLSKADY | |
| | EKHKVYACEVTHQGLSSPVTKSFNRGEC | |
| PC-11 HC | QVQLVESGGGVVQPGRSLRLSCAASGFAFSRYGM | 168 |
| albumin binder: HE-1 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: non-cleavable | DFLYYYYYGMDVWGQGTTVTVSSASTKGPSVFP | |
| CD28 scFv: wt Vl-linker-Vh | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-12 LC | EVQLVESGGGLVQPGGSLRLSCAASGSTFYTAVM | 169 |
| albumin binder: HE-1 | GWVRQAPGKGLEWVAAIRWTALTTSYADSVKG | |
| CD28 mask: Peptide-41 | RFTISRDGAKTTLYLQMNSLRPEDTAVYYCAARG | |
| linker: non-cleavable | TLGLFTTADSYDYWGQGTLVTVSSGGGGSGGGS | |
| CD28 scFv: disulfide Vl-linker- | GGMDWCPRDLWVHCFAGGGGSGGGSGGGGSGG | |
| Vh | ASSGAGGSGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGCGTKVEIKGGGGSGGGGSGGGGSQV | |
| | QLVQSGAEVKKPGASVKVSCKASGYTFTSYYIHW | |
| | VRQAPGQCLEWIGSIYPGNVNTNYNEKFKDRATL | |
| | TVDTSISTAYMELSRLRSDDTAVYFCTRSHYGLD | |
| | WNFDVWGQGTTVTVSSGGGGSDIQMTQSPSSLSA | |
| | SVGDRVTITCRASQGISNYLAWYQQKTGKVPKFL | |
| | IYEASTLQSGVPSRFSGGGSGTDFTLTISSLQPEDV | |
| | ATYYCQNYNSAPFTFGPGTKVDIKRTVAAPSVFIF | |
| | PPSDEQLKSGTASVVCLLNNFYPREAKVQWKVD | |
| | NALQSGNSQESVTEQDSKDSTYSLSSTLTLSKADY | |
| | EKHKVYACEVTHQGLSSPVTKSFNRGEC | |
| PC-12 HC | QVQLVESGGGVVQPGRSLRLSCAASGFAFSRYGM | 170 |
| albumin binder: HE-1 | HWVRQAPGKGLEWVAVIWYDGSNKYYADSVKG | |
| CD28 mask: Peptide-41 | RFTISRDNSKNTQYLQMNSLRAEDTAVYYCARGG | |
| linker: non-cleavable | DFLYYYYYGMDVWGQGTTVTVSSASTKGPSVFP | |
| CD28 scFv: disulfide Vl-linker- | LAPSSKSTSGGTAALGCLVKDYFPEPVTVSWNSG | |
| Vh | ALTSGVHTFPAVLQSSGLYSLSSVVTVPSSSLGTQ | |
| | TYICNVNHKPSNTKVDKKVEPKSC | |
| PC-13 LC | EVQLVESGGGLVQPGGSLRLSCAASGSTFYTAVM | 171 |
| albumin binder: HE-1 | GWVRQAPGKGLEWVAAIRWTALTTSYADSVKG | |
| CD28 mask: Peptide-71 | RFTISRDGAKTTLYLQMNSLRPEDTAVYYCAARG | |
| linker: Linker 29 | TLGLFTTADSYDYWGQGTLVTVSSGGGGSGGGS | |
| CD28 scFv: wt Vh-linker-Vl | GGIDWCPRYLWDECYNAGSGGSGGLSGRSDAGS | |
| | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-13 HC | QVQLVESGGGVVQPGRSLRLSCAASGFAFSRYGM | 172 |
| albumin binder: HE-1 | HWVRQAPGKGLEWVAVIWYDGSNKYYADSVKG | |
| CD28 mask: Peptide-71 | RFTISRDNSKNTQYLQMNSLRAEDTAVYYCARGG | |
| linker: Linker 29 | DFLYYYYYGMDVWGQGTTVTVSSASTKGPSVFP | |
| CD28 scFv: wt Vh-linker-Vl | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-14 LC | EVQLVESGGGLVQPGGSLRLSCAASGSTFYTAVM | 173 |
| albumin binder: HE-1 | GWVRQAPGKGLEWVAAIRWTALTTSYADSVKG | |
| CD28 mask: Peptide-71 | RFTISRDGAKTTLYLQMNSLRPEDTAVYYCAARG | |
| linker: non-cleavable | TLGLFTTADSYDYWGQGTLVTVSSGGGGSGGGS | |
| CD28 scFv: wt Vh-linker-Vl | GGIDWCPRYLWDECYNAGGGSGGGSGGGGSGG | |
| | ASSGAGGSGGGSQVQLVQSGAEVKKPGASVKVS | |
| | CKASGYTFTSYYIHWVRQAPGQGLEWIGSIYPGN | |
| | VNTNYNEKFKDRATLTVDTSISTAYMELSRLRSD | |
| | DTAVYFCTRSHYGLDWNFDVWGQGTTVTVSSGG | |
| | GGSGGGGSGGGGSDIQMTQSPSSLSASVGDRVTIT | |
| | CHASQNIYVWLNWYQQKPGKAPKLLIYKASNLH | |
| | TGVPSRFSGSGSGTDFTLTISSLQPEDFATYYCQQ | |
| | GQTYPYTFGGGTKVEIKGGGGSDIQMTQSPSSLSA | |
| | SVGDRVTITCRASQGISNYLAWYQQKTGKVPKFL | |
| | IYEASTLQSGVPSRFSGGGSGTDFTLTISSLQPEDV | |
| | ATYYCQNYNSAPFTFGPGTKVDIKRTVAAPSVFIF | |
| | PPSDEQLKSGTASVVCLLNNFYPREAKVQWKVD | |
| | NALQSGNSQESVTEQDSKDSTYSLSSTLTLSKADY | |
| | EKHKVYACEVTHQGLSSPVTKSFNRGEC | |
| PC-14 HC | QVQLVESGGGVVQPGRSLRLSCAASGFAFSRYGM | 174 |
| albumin binder: HE-1 | HWVRQAPGKGLEWVAVIWYDGSNKYYADSVKG | |
| CD28 mask: Peptide-71 | RFTISRDNSKNTQYLQMNSLRAEDTAVYYCARGG | |
| linker: non-cleavable | DFLYYYYYGMDVWGQGTTVTVSSASTKGPSVFP | |
| CD28 scFv: wt Vh-linker-Vl | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-15 LC | EVQLVESGGGLVQPGGSLRLSCAASGSTFYTAVM | 175 |
| albumin binder: HE-1 | GWVRQAPGKGLEWVAAIRWTALTTSYADSVKG | |
| CD28 mask: Peptide-71 | RFTISRDGAKTTLYLQMNSLRPEDTAVYYCAARG | |
| linker: Linker 29 | TLGLFTTADSYDYWGQGTLVTVSSGGGGSGGGS | |
| CD28 scFv: disulfide Vh-linker- | GGIDWCPRYLWDECYNAGSGGSGGLSGRSDAGS | |
| Vl | PLGLAGSGGGSQVQLVQSGAEVKKPGASVKVSC | |
| | KASGYTFTSYYIHWVRQAPGQCLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGCGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-15 HC | QVQLVESGGGVVQPGRSLRLSCAASGFAFSRYGM | 176 |
| albumin binder: HE-1 | HWVRQAPGKGLEWVAVIWYDGSNKYYADSVKG | |
| CD28 mask: Peptide-71 | RFTISRDNSKNTQYLQMNSLRAEDTAVYYCARGG | |
| linker: Linker 29 | DFLYYYYYGMDVWGQGTTVTVSSASTKGPSVFP | |
| CD28 scFv: disulfide Vh-linker- | LAPSSKSTSGGTAALGCLVKDYFPEPVTVSWNSG | |
| Vl | ALTSGVHTFPAVLQSSGLYSLSSVVTVPSSSLGTQ | |
| | TYICNVNHKPSNTKVDKKVEPKSC | |
| PC-16 LC | EVQLVESGGGLVQPGGSLRLSCAASGSTFYTAVM | 177 |
| albumin binder: HE-1 | GWVRQAPGKGLEWVAAIRWTALTTSYADSVKG | |
| CD28 mask: Peptide-71 | RFTISRDGAKTTLYLQMNSLRPEDTAVYYCAARG | |
| linker: non-cleavable | TLGLFTTADSYDYWGQGTLVTVSSGGGGSGGGS | |
| CD28 scFv: disulfide Vh-linker- | GGIDWCPRYLWDECYNAGGGSGGGSGGGGSGG | |
| Vl | ASSGAGGSGGGSQVQLVQSGAEVKKPGASVKVS | |
| | CKASGYTFTSYYIHWVRQAPGQCLEWIGSIYPGN | |
| | VNTNYNEKFKDRATLTVDTSISTAYMELSRLRSD | |
| | DTAVYFCTRSHYGLDWNFDVWGQGTTVTVSSGG | |
| | GGSGGGGSGGGGSDIQMTQSPSSLSASVGDRVTIT | |
| | CHASQNIYVWLNWYQQKPGKAPKLLIYKASNLH | |
| | TGVPSRFSGSGSGTDFTLTISSLQPEDFATYYCQQ | |
| | GQTYPYTFGCGTKVEIKGGGGSDIQMTQSPSSLSA | |
| | SVGDRVTITCRASQGISNYLAWYQQKTGKVPKFL | |
| | IYEASTLQSGVPSRFSGGGSGTDFTLTISSLQPEDV | |
| | ATYYCQNYNSAPFTFGPGTKVDIKRTVAAPSVFIF | |
| | PPSDEQLKSGTASVVCLLNNFYPREAKVQWKVD | |
| | NALQSGNSQESVTEQDSKDSTYSLSSTLTLSKADY | |
| | EKHKVYACEVTHQGLSSPVTKSFNRGEC | |
| PC-16 HC | QVQLVESGGGVVQPGRSLRLSCAASGFAFSRYGM | 178 |
| albumin binder: HE-1 | HWVRQAPGKGLEWVAVIWYDGSNKYYADSVKG | |
| CD28 mask: Peptide-71 | RFTISRDNSKNTQYLQMNSLRAEDTAVYYCARGG | |
| linker: non-cleavable | DFLYYYYYGMDVWGQGTTVTVSSASTKGPSVFP | |
| CD28 scFv: disulfide Vh-linker- | LAPSSKSTSGGTAALGCLVKDYFPEPVTVSWNSG | |
| Vl | | |
| | ALTSGVHTFPAVLQSSGLYSLSSVVTVPSSSLGTQ | |
| | TYICNVNHKPSNTKVDKKVEPKSC | |
| PC-17 LC | EVQLVESGGGLVQPGGSLRLSCAASGSTFYTAVM | 4330 |
| Albumin binder: HE-1 | GWVRQAPGKGLEWVAAIRWTALTTSYADSVKG | |
| CD28 mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| | GGMDWCPRDLWVHCFAGSSGGSGGLSGRSDAGS | |
| | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-17 HC | QVQLVESGGGVVQPGRSLRLSCAASGFAFSRYGM | 4331 |
| Albumin binder: HE-1 | HWVRQAPGKGLEWVAVIWYDGSNKYYADSVKG | |
| CD28 mask: Peptide-41 | RFTISRDNSKNTQYLQMNSLRAEDTAVYYCARGG | |
| CD28 binder: scFv Vh-linker-Vl | DFLYYYYYGMDVWGQGTTVTVSSASTKGPSVFP | |
| | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-18 LC | EVQLVESGGGLVQPGNSLRLSCAASGFTFSSFGMS | 4332 |
| Albumin binder: HE-4 | WVRQAPGKGLEWVSSISGSGSDTLYADSVKGRFT | |
| CD28 Mask: Peptide-71 | ISRDNAKTTLYLQMNSLRPEDTAVYYCTIGGSLSR | |
| CD28 binder: scFv Vh-linker-Vl | SSQGTLVTVSSGGGGSGGGSGGIDWCPRYLWDEC | |
| | YNAGSGGSGGLSGRSDAGSPLGLAGSGGGSQVQL | |
| | VQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-18 HC | QVQLVESGGGVVQPGRSLRLSCAASGFAFSRYGM | 4333 |
| Albumin binder: HE-4 | HWVRQAPGKGLEWVAVIWYDGSNKYYADSVKG | |
| CD28 Mask: Peptide-71 | RFTISRDNSKNTQYLQMNSLRAEDTAVYYCARGG | |
| CD28 binder: scFv Vh-linker-Vl | DFLYYYYYGMDVWGQGTTVTVSSASTKGPSVFP | |
| | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-19 LC | EVQLVESGGGLVQPGNSLRLSCAASGFTFSSFGMS | 4334 |
| Albumin binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vh-linker-Vl | SSQGTLVTVSSGGGGSGGGSGGMDWCPRDLWVH | |
| | CFAGSSGGSGGLSGRSDAGSPLGLAGSGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-19 HC | QVQLVESGGGVVQPGRSLRLSCAASGFAFSRYGM | 4335 |
| Albumin binder: HE-4 | HWVRQAPGKGLEWVAVIWYDGSNKYYADSVKG | |
| CD28 Mask: Peptide-41 | RFTISRDNSKNTQYLQMNSLRAEDTAVYYCARGG | |
| CD28 binder: scFv Vh-linker-Vl | DFLYYYYYGMDVWGQGTTVTVSSASTKGPSVFP | |
| | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-20 LC | EVQLVESGGGLVQPGGSLRLSCAASGSTFYTAVM | 4336 |
| Albumin Binder: HE-1 | GWVRQAPGKGLEWVAAIRWTALTTSYADSVKG | |
| CD28 Mask: Peptide-71 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| | GGIDWCPRYLWDECYNAGSGGSGGLSGRSDAGS | |
| | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-20 HC | QVQLVESGGGVVQPGRSLRLSCAASGFAFSRYGM | 4337 |
| Albumin Binder: HE-1 | HWVRQAPGKGLEWVAVIWYDGSNKYYADSVKG | |
| CD28 Mask: Peptide-71 | RFTISRDNSKNTQYLQMNSLRAEDTAVYYCARGG | |
| CD28 binder: scFv Vl-linker-Vh | DFLYYYYYGMDVWGQGTTVTVSSASTKGPSVFP | |
| | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKKVEPKSC | |
| PC-21 LC | EVQLVESGGGLVQPGGSLRLSCAASGSTFYTAVM | 4338 |
| Albumin Binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-219 | GGMDWCPRDLWVHCFAGSSGGSGGLSGRSDAGS | |
| PSMA binder: wildtype | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-21 HC | GGHVKLCKPAWMCQGLGGGSGGLSGRSDAGSPL | 4339 |
| Albumin Binder: HE-1 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-219 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-22 LC | EVQLVESGGGLVQPGGSLRLSCAASGSTFYTAVM | 4340 |
| Albumin Binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-219 | GGMDWCPRDLWVHCFAGSSGGSGGLSGRSDAGS | |
| PSMA binder: R19S mutant | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-22 HC | GGHVKLCKPAWMCQGLGGGSGGLSGRSDAGSPL | 4341 |
| Albumin Binder: HE-1 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-219 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-23 LC | EVQLVESGGGLVQPGNSLRLSCAASGFTFSSFGMS | 4342 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vh-linker-Vl | SSQGTLVTVSSGGGGSGGGSGGMDWCPRDLWVH | |
| PSMA Mask: Peptide-219 | CFAGSSGGSGGLSGRSDAGSPLGLAGSGGGSQVQ | |
| PSMA binder: wildtype | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-23 HC | GGHVKLCKPAWMCQGLGGGSGGLSGRSDAGSPL | 4343 |
| Albumin Binder: HE-4 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-219 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-24 LC | EVQLVESGGGLVQPGNSLRLSCAASGFTFSSFGMS | 4344 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vh-linker-Vl | SSQGTLVTVSSGGGGSGGGSGGMDWCPRDLWVH | |
| PSMA Mask: Peptide-219 | CFAGSSGGSGGLSGRSDAGSPLGLAGSGGGSQVQ | |
| PSMA binder: R19S mutant | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-24 HC | GGHVKLCKPAWMCQGLGGGSGGLSGRSDAGSPL | 4345 |
| Albumin Binder: HE-4 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-219 | VYYCARGGDFLYYYYYGMDVWGQGTTVTVSSA | |
| PSMA binder; R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-25 LC | LVQPGGSLRLSCAASGSTFYTAVMGWVRQAPGK | 4346 |
| Albumin Binder: HE-1 | GLEWVAAIRWTALTTSYADSVKGRFTISRDGAKT | |
| CD28 Mask: Peptide-41 | TLYLQMNSLRPEDTAVYYCAARGTLGLFTTADSY | |
| CD28 binder: scFv Vl-linker-Vh | DYWGQGTLVTVSSGGGGSGGGSGGMDWCPRDL | |
| PSMA Mask: Peptide-219 | WVHCFAGSSGGSGGLSGRSDAGSPLGLAGSGGGS | |
| PSMA binder: wildtype | DIQMTQSPSSLSASVGDRVTITCHASQNIYVWLN | |
| | WYQQKPGKAPKLLIYKASNLHTGVPSRFSGSGSG | |
| | TDFTLTISSLQPEDFATYYCQQGQTYPYTFGGGTK | |
| | VEIKGGGGSGGGGSGGGGSQVQLVQSGAEVKKP | |
| | GASVKVSCKASGYTFTSYYIHWVRQAPGQGLEWI | |
| | GSIYPGNVNTNYNEKFKDRATLTVDTSISTAYME | |
| | LSRLRSDDTAVYFCTRSHYGLDWNFDVWGQGTT | |
| | VTVSSGGGGSDIQMTQSPSSLSASVGDRVTITCRA | |
| | SQGISNYLAWYQQKTGKVPKFLIYEASTLQSGVPS | |
| | RFSGGGSGTDFTLTISSLQPEDVATYYCQNYNSAP | |
| | FTFGPGTKVDIKRTVAAPSVFIFPPSDEQLKSGTAS | |
| | VVCLLNNFYPREAKVQWKVDNALQSGNSQESVT | |
| | EQDSKDSTYSLSSTLTLSKADYEKHKVYACEVTH | |
| | QGLSSPVTKSFNRGEC | |
| PC-25 HC | GGHVKLCKPAWMCQGLGGGSGGLSGRSDAGSPL | 4347 |
| Albumin Binder: HE-1 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-219 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-26 LC | EVQLVESGGGLVQPGGSLRLSCAASGSTFYTAVM | 4348 |
| Albumin Binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA Mask: Peptide-219 | GGMDWCPRDLWVHCFAGSSGGSGGLSGRSDAGS | |
| PSMA binder: R19S mutant | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-26 HC | GGHVKLCKPAWMCQGLGGGSGGLSGRSDAGSPL | 4349 |
| Albumin Binder: HE-1 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-219 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-27 LC | EVQLVESGGGLVQPGNSLRLSCAASGFTFSSFGMS | 4350 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-219 | CFAGSSGGSGGLSGRSDAGSPLGLAGSGGGSDIQ | |
| PSMA binder: wildtype | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-27 HC | GGHVKLCKPAWMCQGLGGGSGGLSGRSDAGSPL | 4351 |
| Albumin Binder: HE-4 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-219 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-28 LC | EVQLVESGGGLVQPGNSLRLSCAASGFTFSSFGMS | 4352 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-219 | CFAGSSGGSGGLSGRSDAGSPLGLAGSGGGSDIQ | |
| PSMA binder: R19S mutant | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-28 HC | GGHVKLCKPAWMCQGLGGGSGGLSGRSDAGSPL | 4353 |
| Albumin Binder: HE-4 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-219 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-29 LC | EVQLVESGGGLVQPGGSLRLSCAASGSTFYTAVM | 4354 |
| Albumin Binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-230 | GGMDWCPRDLWVHCFAGSSGGSGGLSGRSDAGS | |
| PSMA binder: wildtype | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-29 HC | GGVTKWCIPPWMCEVTGGGSGGLSGRSDAGSPL | 4355 |
| Albumin Binder: HE-1 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-230 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-30 LC | EVQLVESGGGLVQPGGSLRLSCAASGSTFYTAVM | 4356 |
| Albumin Binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-230 | GGMDWCPRDLWVHCFAGSSGGSGGLSGRSDAGS | |
| PSMA binder: R19S mutant | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-30 HC | GGVTKWCIPPWMCEVTGGGSGGLSGRSDAGSPL | 4357 |
| Albumin Binder: HE-1 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-230 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-31 LC | EVQLVESGGGLVQPGNSLRLSCAASGFTFSSFGMS | 4358 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vh-linker-Vl | SSQGTLVTVSSGGGGSGGGSGGMDWCPRDLWVH | |
| PSMA Mask: Peptide-230 | CFAGSSGGSGGLSGRSDAGSPLGLAGSGGGSQVQ | |
| PSMA binder: wildtype | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-31 HC | GGVTKWCIPPWMCEVTGGGSGGLSGRSDAGSPL | 4359 |
| Albumin Binder: HE-4 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-230 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-32 LC | EVQLVESGGGLVQPGNSLRLSCAASGFTFSSFGMS | 4360 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vh-linker-Vl | SSQGTLVTVSSGGGGSGGGSGGMDWCPRDLWVH | |
| PSMA Mask: Peptide-230 | CFAGSSGGSGGLSGRSDAGSPLGLAGSGGGSQVQ | |
| PSMA binder: R19S mutant | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-32 HC | GGVTKWCIPPWMCEVTGGGSGGLSGRSDAGSPL | 4361 |
| Albumin Binder: HE-4 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-230 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-33 LC | EVQLVESGGGLVQPGGSLRLSCAASGSTFYTAVM | 4362 |
| Albumin Binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA Mask: Peptide-230 | GGMDWCPRDLWVHCFAGSSGGSGGLSGRSDAGS | |
| PSMA binder: wildtype | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-33 HC | GGVTKWCIPPWMCEVTGGGSGGLSGRSDAGSPL | 4363 |
| Albumin Binder: HE-1 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-230 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-34 LC | EVQLVESGGGLVQPGGSLRLSCAASGSTFYTAVM | 4364 |
| Albumin Binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA Mask: Peptide-230 | GGMDWCPRDLWVHCFAGSSGGSGGLSGRSDAGS | |
| PSMA binder: R19S mutant | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-34 HC | GGVTKWCIPPWMCEVTGGGSGGLSGRSDAGSPL | 4365 |
| Albumin Binder: HE-1 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-230 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-35 LC | EVQLVESGGGLVQPGNSLRLSCAASGFTFSSFGMS | 4366 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-230 | CFAGSSGGSGGLSGRSDAGSPLGLAGSGGGSDIQ | |
| PSMA binder: wildtype | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-35 HC | GGVTKWCIPPWMCEVTGGGSGGLSGRSDAGSPL | 4367 |
| Albumin Binder: HE-4 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-230 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-36 LC | EVQLVESGGGLVQPGNSLRLSCAASGFTFSSFGMS | 4368 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-230 | CFAGSSGGSGGLSGRSDAGSPLGLAGSGGGSDIQ | |
| PSMA binder: wildtype | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-36 HC | GGVTKWCIPPWMCEVTGGGSGGLSGRSDAGSPL | 4369 |
| Albumin Binder: HE-4 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask:2815 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-37 LC | EVQLVESGGGLVQPGNSLRLSCAASGFTFSSFGMS | 4370 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-230 | CFAGSSGGSGGLSGRSDAGSPLGLAGSGGGSDIQ | |
| PSMA binder: R19S mutant | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-37 HC | GGVTKWCIPPWMCEVTGGGSGGLSGRSDAGSPL | 4371 |
| Albumin Binder: HE-4 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-230 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-38 LCAlbumin Binder: HE-1 | EVQLVESGGGLVQPGGSLRLSCAASGSTFYTAVM | 4372 |
| CD28 Mask: Peptide-41 | GWVRQAPGKGLEWVAAIRWTALTTSYADSVKG | |
| CD28 binder: scFv Vh-linker-Vl | RFTISRDGAKTTLYLQMNSLRPEDTAVYYCAARG | |
| PSMA Mask: Peptide-233 | TLGLFTTADSYDYWGQGTLVTVSSGGGGSGGGS | |
| PSMA binder: wildtype | GGMDWCPRDLWVHCFAGSSGGSGGLSGRSDAGS | |
| | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-38 HCAlbumin Binder: HE-1 | GGGEKWCIPDWMCSGFGGGSGGLSGRSDAGSPL | 4373 |
| CD28 Mask: Peptide-41 | GLAGSGGSQVQLVESGGGVVQPGRSLRLSCAASG | |
| CD28 binder: scFv Vh-linker-Vl | FAFSRYGMHWVRQAPGKGLEWVAVIWYDGSNK | |
| PSMA Mask: Peptide-233 | YYADSVKGRFTISRDNSKNTQYLQMNSLRAEDTA | |
| PSMA binder: wildtype | VYYCARGGDFLYYYYYGMDVWGQGTTVTVSSA | |
| | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-39 LCAlbumin Binder: HE-1 | EVQLVESGGGLVQPGGSLRLSCAASGSTFYTAVM | 4374 |
| CD28 Mask: Peptide-41 | GWVRQAPGKGLEWVAAIRWTALTTSYADSVKG | |
| CD28 binder: scFv Vh-linker-Vl | RFTISRDGAKTTLYLQMNSLRPEDTAVYYCAARG | |
| PSMA Mask: Peptide-233 | TLGLFTTADSYDYWGQGTLVTVSSGGGGSGGGS | |
| PSMA binder: R19S mutant | GGMDWCPRDLWVHCFAGSSGGSGGLSGRSDAGS | |
| | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-39 HCAlbumin Binder: HE-1 | GGGEKWCIPDWMCSGFGGGSGGLSGRSDAGSPL | 4375 |
| CD28 Mask: Peptide-41 | GLAGSGGSQVQLVESGGGVVQPGRSLSLSCAASG | |
| CD28 binder: scFv Vh-linker-Vl | FAFSRYGMHWVRQAPGKGLEWVAVIWYDGSNK | |
| PSMA Mask: Peptide-233 | YYADSVKGRFTISRDNSKNTQYLQMNSLRAEDTA | |
| PSMA binder: R19S mutant | VYYCARGGDFLYYYYYGMDVWGQGTTVTVSSA | |
| | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-40 LC | EVQLVESGGGLVQPGNSLRLSCAASGFTFSSFGMS | 4376 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vh-linker-Vl | SSQGTLVTVSSGGGGSGGGSGGMDWCPRDLWVH | |
| PSMA Mask: Peptide-233 | CFAGSSGGSGGLSGRSDAGSPLGLAGSGGGSQVQ | |
| PSMA binder: wildtype | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-40 HC | GGGEKWCIPDWMCSGFGGGSGGLSGRSDAGSPL | 4377 |
| Albumin Binder: HE-4 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-233 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-41 LC | EVQLVESGGGLVQPGNSLRLSCAASGFTFSSFGMS | 4378 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vh-linker-Vl | SSQGTLVTVSSGGGGSGGGSGGMDWCPRDLWVH | |
| PSMA Mask: Peptide-233 | CFAGSSGGSGGLSGRSDAGSPLGLAGSGGGSQVQ | |
| PSMA binder: R19S mutant | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-41 HCAlbumin Binder: HE-4 | GGGEKWCIPDWMCSGFGGGSGGLSGRSDAGSPL | 4379 |
| CD28 Mask: Peptide-41 | GLAGSGGSQVQLVESGGGVVQPGRSLSLSCAASG | |
| CD28 binder: scFv Vh-linker-Vl | FAFSRYGMHWVRQAPGKGLEWVAVIWYDGSNK | |
| PSMA Mask: Peptide-233 | YYADSVKGRFTISRDNSKNTQYLQMNSLRAEDTA | |
| PSMA binder: R19S mutant | VYYCARGGDFLYYYYYGMDVWGQGTTVTVSSA | |
| | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-42 LCAlbumin Binder: HE-1 | EVQLVESGGGLVQPGGSLRLSCAASGSTFYTAVM | 4380 |
| CD28 Mask: Peptide-41 | GWVRQAPGKGLEWVAAIRWTALTTSYADSVKG | |
| CD28 binder: scFv Vl-linker-Vh | RFTISRDGAKTTLYLQMNSLRPEDTAVYYCAARG | |
| PSMA Mask: Peptide-233 | TLGLFTTADSYDYWGQGTLVTVSSGGGGSGGGS | |
| PSMA binder: wildtype | GGMDWCPRDLWVHCFAGSSGGSGGLSGRSDAGS | |
| | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-42 HC | GGGEKWCIPDWMCSGFGGGSGGLSGRSDAGSPL | 4381 |
| Albumin Binder: HE-1 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-233 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-43 LC | EVQLVESGGGLVQPGGSLRLSCAASGSTFYTAVM | 4382 |
| Albumin Binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA Mask: Peptide-233 | GGMDWCPRDLWVHCFAGSSGGSGGLSGRSDAGS | |
| PSMA binder: R19S mutant | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-43 HC | GGGEKWCIPDWMCSGFGGGSGGLSGRSDAGSPL | 4383 |
| Albumin Binder: HE-1 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-233 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-44 LC | EVQLVESGGGLVQPGNSLRLSCAASGFTFSSFGMS | 4384 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-233 | CFAGSSGGSGGLSGRSDAGSPLGLAGSGGGSDIQ | |
| PSMA binder: wildtype | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-44 HC | GGGEKWCIPDWMCSGFGGGSGGLSGRSDAGSPL | 4385 |
| Albumin Binder: HE-4 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-233 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-45 LC | EVQLVESGGGLVQPGNSLRLSCAASGFTFSSFGMS | 4386 |
| Albumin Binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-233 | CFAGSSGGSGGLSGRSDAGSPLGLAGSGGGSDIQ | |
| PSMA binder: R.19S mutant | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-45 HC | GGGEKWCIPDWMCSGFGGGSGGLSGRSDAGSPL | 4387 |
| Albumin Binder: HE-4 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-233 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-46 LC | EVQLVESGGGLVQPGGSLRLSCAASGSTFYTAVM | 4388 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-268 | GGMDWCPRDLWVHCFAGSSGGSGGLSGRSDAGS | |
| PSMA binder: wildtype | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-46 HC | GGVEKVCKPVWMCVGFGGGSGGLSGRSDAGSPL | 4389 |
| Album binder: HB-1 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-268 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-47 LC | EVQLVESGGGLVQPGGSLRLSCAASGSTFYTAVM | 4390 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-268 | GGMDWCPRDLWVHCFAGSSGGSGGLSGRSDAGS | |
| PSMA binder: R19S mutant | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-47 HC | GGVEKVCKPVWMCVGFGGGSGGLSGRSDAGSPL | 4391 |
| Album binder: HE-1 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-268 | VYYCARGGDFLYYYYYGMDVWGQGTTVTVSSA | |
| PSMA binder: R19S mutant | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-48 LCAlbum binder: HE-4 | EVQLVESGGGLVQPGNSLRLSCAASGFTFSSFGMS | 4392 |
| CD28 Mask: Peptide-41 | WVRQAPGKGLEWVSSISGSGSDTLYADSVKGRFT | |
| CD28 binder: scFv Vh-linker-Vl | ISRDNAKTTLYLQMNSLRPEDTAVYYCTIGGSLSR | |
| PSMA Mask: Peptide-268 | SSQGTLVTVSSGGGGSGGGSGGMDWCPRDLWVH | |
| PSMA binder: wildtype | CFAGSSGGSGGLSGRSDAGSPLGLAGSGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-48 HC | GGVEKVCKPVWMCVGFGGGSGGLSGRSDAGSPL | 4393 |
| Album binder: HE-4 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vh-linker-Vl | YYADSVKGRFTISRDNSKNTQYLQMNSLRAEDTA | |
| PSMA Mask: Peptide-268 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-49 LCAlbum binder: HE-4 | EVQLVESGGGLVQPGNSLRLSCAASGFTFSSFGMS | 4394 |
| CD28 Mask: Peptide-41 | WVRQAPGKGLEWVSSISGSGSDTLYADSVKGRFT | |
| CD28 binder: scFv Vh-linker-Vl | ISRDNAKTTLYLQMNSLRPEDTAVYYCTIGGSLSR | |
| PSMA Mask: Peptide-268 | SSQGTLVTVSSGGGGSGGGSGGMDWCPRDLWVH | |
| PSMA binder: R19S mutant | CFAGSSGGSGGLSGRSDAGSPLGLAGSGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-49 HCAlbum binder: HE-4 | GGVEKVCKPVWMCVGFGGGSGGLSGRSDAGSPL | 4395 |
| CD28 Mask: Peptide-41 | GLAGSGGSQVQLVESGGGVVQPGRSLSLSCAASG | |
| CD28 binder: scFv Vh-linker-Vl | FAFSRYGMHWVRQAPGKGLEWVAVIWYDGSNK | |
| PSMA Mask: Peptide-268 | YYADSVKGRFTISRDNSKNTQYLQMNSLRAEDTA | |
| PSMA binder: R19S mutant | VYYCARGGDFLYYYYYGMDVWGQGTTVTVSSA | |
| | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-50 LCAlbum binder: HE-1 | EVQLVESGGGLVQPGGSLRLSCAASGSTFYTAVM | 4396 |
| CD28 Mask: Peptide-41 | GWVRQAPGKGLEWVAAIRWTALTTSYADSVKG | |
| CD28 binder: scFv Vl-linker-Vh | RFTISRDGAKTTLYLQMNSLRPEDTAVYYCAARG | |
| PSMA Mask: Peptide-268 | TLGLFTTADSYDYWGQGTLVTVSSGGGGSGGGS | |
| PSMA binder: wildtype | GGMDWCPRDLWVHCFAGSSGGSGGLSGRSDAGS | |
| | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-50 HCAlbum binder: HE-1 | GGVEKVCKPVWMCVGFGGGSGGLSGRSDAGSPL | 4397 |
| CD28 Mask: Peptide-41 | GLAGSGGSQVQLVESGGGVVQPGRSLRLSCAASG | |
| CD28 binder: scFv Vl-linker-Vh | FAFSRYGMHWVRQAPGKGLEWVAVIWYDGSNK | |
| PSMA Mask: Peptide-268 | YYADSVKGRFTISRDNSKNTQYLQMNSLRAEDTA | |
| PSMA binder: wildtype | VYYCARGGDFLYYYYYGMDVWGQGTTVTVSSA | |
| | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-51 LCAlbum binder: HE-1 | EVQLVESGGGLVQPGGSLRLSCAASGSTFYTAVM | 4398 |
| CD28 Mask: Peptide-41 | GWVRQAPGKGLEWVAAIRWTALTTSYADSVKG | |
| CD28 binder: scFv Vl-linker-Vh | RFTISRDGAKTTLYLQMNSLRPEDTAVYYCAARG | |
| PSMA Mask: Peptide-268 | TLGLFTTADSYDYWGQGTLVTVSSGGGGSGGGS | |
| PSMA binder: R19S mutant | GGMDWCPRDLWVHCFAGSSGGSGGLSGRSDAGS | |
| | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-51 HCAlbum binder: HE-1 | GGVEKVCKPVWMCVGFGGGSGGLSGRSDAGSPL | 4399 |
| CD28 Mask: Peptide-41 | GLAGSGGSQVQLVESGGGVVQPGRSLSLSCAASG | |
| CD28 binder: scFv Vl-linker-Vh | FAFSRYGMHWVRQAPGKGLEWVAVIWYDGSNK | |
| PSMA Mask: Peptide-268 | YYADSVKGRFTISRDNSKNTQYLQMNSLRAEDTA | |
| PSMA binder: R19S mutant | VYYCARGGDFLYYYYYGMDVWGQGTTVTVSSA | |
| | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-52 LCAlbum binder: HE-4 | EVQLVESGGGLVQPGNSLRLSCAASGFTFSSFGMS | 4400 |
| CD28 Mask: Peptide-41 | WVRQAPGKGLEWVSSISGSGSDTLYADSVKGRFT | |
| CD28 binder: scFv Vl-linker-Vh | ISRDNAKTTLYLQMNSLRPEDTAVYYCTIGGSLSR | |
| PSMA Mask: Peptide-268 | SSQGTLVTVSSGGGGSGGGSGGMDWCPRDLWVH | |
| PSMA binder: wildtype | CFAGSSGGSGGLSGRSDAGSPLGLAGSGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-52 HC | GGVEKVCKPVWMCVGFGGGSGGLSGRSDAGSPL | 4401 |
| Album binder: HE-4 | GLAGSGGSQVQLVESGGGVVQPGRSLRLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-268 | VYYCARGGDFLYYYYYGMDVWGQGTTVTVSSA | |
| PSMA binder: wildtype | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-53 LC | EVQLVESGGGLVQPGNSLRLSCAASGFTFSSFGMS | 4402 |
| Album binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-268 | CFAGSSGGSGGLSGRSDAGSPLGLAGSGGGSDIQ | |
| PSMA binder: R19S mutant | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-53 HC | GGVEKVCKPVWMCVGFGGGSGGLSGRSDAGSPL | 4403 |
| Album binder: HE-4 | GLAGSGGSQVQLVESGGGVVQPGRSLSLSCAASG | |
| CD28 Mask: Peptide-41 | FAFSRYGMHWVRQAPGKGLEWVAVIWYDGSNK | |
| CD28 binder: scFv Vl-linker-Vh | YYADSVKGRFTISRDNSKNTQYLQMNSLRAEDTA | |
| PSMA Mask: Peptide-268 | VYYCARGGDFLYYYYYGMDVWGQGTTVTVSSA | |
| PSMA binder: R19S mutant : | STKGPSVFPLAPSSKSTSGGTAALGCLVKDYFPEP | |
| | VTVSWNSGALTSGVHTFPAVLQSSGLYSLSSVVT | |
| | VPSSSLGTQTYICNVNHKPSNTKVDKKVEPKSC | |
| PC-54 LC | EVQLVESGGGLVQPGGSLRLSCAASGSTFYTAVM | 4404 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-271 | GGMDWCPRDLWVHCFAGSSGGSGGLSGRSDAGS | |
| PSMA binder: wildtype | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-54 HCAlbum binder: HE-1 | GGEDILCKPKWMCEGFGGGSGGLSGRSDAGSPLG | 4405 |
| CD28 Mask: Peptide-41 | LAGSGGSQVQLVESGGGVVQPGRSLRLSCAASGF | |
| CD28 binder: scFv Vh-linker-Vl | AFSRYGMHWVRQAPGKGLEWVAVIWYDGSNKY | |
| PSMA Mask: Peptide-271 | YADSVKGRFTISRDNSKNTQYLQMNSLRAEDTAV | |
| PSMA binder: wildtype | YYCARGGDFLYYYYYGMDVWGQGTTVTVSSAS | |
| | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-55 LC | EVQLVESGGGLVQPGGSLRLSCAASGSTFYTAVM | 4406 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-271 | GGMDWCPRDLWVHCFAGSSGGSGGLSGRSDAGS | |
| PSMA binder: R19S mutant | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-55 HC | GGEDILCKPKWMCEGFGGGSGGLSGRSDAGSPLG | 4407 |
| Album binder: HE-1 | LAGSGGSQVQLVESGGGVVQPGRSLSLSCAASGF | |
| CD28 Mask: Peptide-41 | AFSRYGMHWVRQAPGKGLEWVAVIWYDGSNKY | |
| CD28 binder: scFv Vh-linker-Vl | YADSVKGRFTISRDNSKNTQYLQMNSLRAEDTAV | |
| PSMA Mask: Peptide-271 | YYCARGGDFLYYYYYGMDVWGQGTTVTVSSAS | |
| PSMA binder: R19S mutant | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-56 LCAlbum binder: HE-4 | EVQLVESGGGLVQPGNSLRLSCAASGFTFSSFGMS | 4408 |
| CD28 Mask: Peptide-41 | WVRQAPGKGLEWVSSISGSGSDTLYADSVKGRFT | |
| CD28 binder: scFv Vh-linker-Vl | ISRDNAKTTLYLQMNSLRPEDTAVYYCTIGGSLSR | |
| PSMA Mask: Peptide-271 | SSQGTLVTVSSGGGGSGGGSGGMDWCPRDLWVH | |
| PSMA binder: wildtype | CFAGSSGGSGGLSGRSDAGSPLGLAGSGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-56 HC | GGEDILCKPKWMCEGFGGGSGGLSGRSDAGSPLG | 4409 |
| Album binder: HE-4 | LAGSGGSQVQLVESGGGVVQPGRSLRLSCAASGF | |
| CD28 Mask: Peptide-41 | AFSRYGMHWVRQAPGKGLEWVAVIWYDGSNKY | |
| CD28 binder: scFv Vh-linker-Vl | YADSVKGRFTISRDNSKNTQYLQMNSLRAEDTAV | |
| PSMA Mask: Peptide-271 | YYCARGGDFLYYYYYGMDVWGQGTTVTVSSAS | |
| PSMA binder: wildtype | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-57 LCAlbum binder: HE-4 | EVQLVESGGGLVQPGNSLRLSCAASGFTFSSFGMS | 4410 |
| CD28 Mask: Peptide-41 | WVRQAPGKGLEWVSSISGSGSDTLYADSVKGRFT | |
| CD28 binder: scFv Vh-linker-Vl | ISRDNAKTTLYLQMNSLRPEDTAVYYCTIGGSLSR | |
| PSMA Mask: Peptide-271 | SSQGTLVTVSSGGGGSGGGSGGMDWCPRDLWVH | |
| PSMA binder: R19S mutant | CFAGSSGGSGGLSGRSDAGSPLGLAGSGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-57 HC | GGEDILCKPKWMCEGFGGGSGGLSGRSDAGSPLG | 4411 |
| Album binder: HE-4 | LAGSGGSQVQLVESGGGVVQPGRSLSLSCAASGF | |
| CD28 Mask: Peptide-41 | AFSRYGMHWVRQAPGKGLEWVAVIWYDGSNKY | |
| CD28 binder: scFv Vh-linker-Vl | YADSVKGRFTISRDNSKNTQYLQMNSLRAEDTAV | |
| PSMA Mask: Peptide-271 | YYCARGGDFLYYYYYGMDVWGQGTTVTVSSAS | |
| PSMA binder: R.19S mutant | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-58 LC | EVQLVESGGGLVQPGGSLRLSCAASGSTFYTAVM | 4412 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA Mask: Peptide-271 | GGMDWCPRDLWVHCFAGSSGGSGGLSGRSDAGS | |
| PSMA binder: wildtype | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-58 HC | GGEDILCKPKWMCEGFGGGSGGLSGRSDAGSPLG | 4413 |
| Album binder: HE-1 | LAGSGGSQVQLVESGGGVVQPGRSLRLSCAASGF | |
| CD28 Mask: Peptide-41 | AFSRYGMHWVRQAPGKGLEWVAVIWYDGSNKY | |
| CD28 binder: scFv Vl-linker-Vh | YADSVKGRFTISRDNSKNTQYLQMNSLRAEDTAV | |
| PSMA Mask: Peptide-271 | YYCARGGDFLYYYYYGMDVWGQGTTVTVSSAS | |
| PSMA binder: wildtype | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-59 LC | EVQLVESGGGLVQPGGSLRLSCAASGSTFYTAVM | 4414 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA Mask: Peptide-271 | GGMDWCPRDLWVHCFAGSSGGSGGLSGRSDAGS | |
| PSMA binder: R19S mutant | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-59 HC | GGEDILCKPKWMCEGFGGGSGGLSGRSDAGSPLG | 4415 |
| Album binder: HE-1 | LAGSGGSQVQLVESGGGVVQPGRSLSLSCAASGF | |
| CD28 Mask: Peptide-41 | AFSRYGMHWVRQAPGKGLEWVAVIWYDGSNKY | |
| CD28 binder: scFv Vl-linker-Vh | YADSVKGRFTISRDNSKNTQYLQMNSLRAEDTAV | |
| PSMA Mask: Peptide-271 | YYCARGGDFLYYYYYGMDVWGQGTTVTVSSAS | |
| PSMA binder: R19S mutant | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-60 LC | EVQLVESGGGLVQPGNSLRLSCAASGFTFSSFGMS | 4416 |
| Album binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-271 | CFAGSSGGSGGLSGRSDAGSPLGLAGSGGGSDIQ | |
| PSMA binder: wildtype | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-60 HC | GGEDILCKPKWMCEGFGGGSGGLSGRSDAGSPLG | 4417 |
| Album binder: HE-4 | LAGSGGSQVQLVESGGGVVQPGRSLRLSCAASGF | |
| CD28 Mask: Peptide-41 | AFSRYGMHWVRQAPGKGLEWVAVIWYDGSNKY | |
| CD28 binder: scFv Vl-linker-Vh | YADSVKGRFTISRDNSKNTQYLQMNSLRAEDTAV | |
| PSMA Mask: Peptide-271 | YYCARGGDFLYYYYYGMDVWGQGTTVTVSSAS | |
| PSMA binder: wildtype | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-61 LC | EVQLVESGGGLVQPGNSLRLSCAASGFTFSSFGMS | 4418 |
| Album binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-271 | CFAGSSGGSGGLSGRSDAGSPLGLAGSGGGSDIQ | |
| PSMA binder: R19S mutant | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-61 HC | GGEDILCKPKWMCEGFGGGSGGLSGRSDAGSPLG | 4419 |
| Album binder: HE-4 | LAGSGGSQVQLVESGGGVVQPGRSLSLSCAASGF | |
| CD28 Mask: Peptide-41 | AFSRYGMHWVRQAPGKGLEWVAVIWYDGSNKY | |
| CD28 binder: scFv Vl-linker-Vh | YADSVKGRFTISRDNSKNTQYLQMNSLRAEDTAV | |
| PSMA Mask: Peptide-271 | YYCARGGDFLYYYYYGMDVWGQGTTVTVSSAS | |
| PSMA binder: R19S mutant | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-62 LC | EVQLVESGGGLVQPGGSLRLSCAASGSTFYTAVM | 4420 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-274 | GGMDWCPRDLWVHCFAGSSGGSGGLSGRSDAGS | |
| PSMA binder: wildtype | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-62 HC | GGITPVCKPLWMCSGLGGGSGGLSGRSDAGSPLG | 4421 |
| Album binder: HE-1 | LAGSGGSQVQLVESGGGVVQPGRSLRLSCAASGF | |
| CD28 Mask: Peptide-41 | AFSRYGMHWVRQAPGKGLEWVAVIWYDGSNKY | |
| CD28 binder: scFv Vh-linker-Vl | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder: wildtype | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-63 LC | EVQLVESGGGLVQPGGSLRLSCAASGSTFYTAVM | 4422 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-274 | GGMDWCPRDLWVHCFAGSSGGSGGLSGRSDAGS | |
| PSMA binder: R19S mutant | PLGLAGSGGGSQVQLVQSGAEVKKPGASVKVSC | |
| | KASGYTFTSYYIHWVRQAPGQGLEWIGSIYPGNV | |
| | NTNYNEKFKDRATLTVDTSISTAYMELSRLRSDD | |
| | TAVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-63 HC | GGITPVCKPLWMCSGLGGGSGGLSGRSDAGSPLG | 4423 |
| Album binder: HE-1 | LAGSGGSQVQLVESGGGVVQPGRSLSLSCAASGF | |
| CD28 Mask: Peptide-41 | AFSRYGMHWVRQAPGKGLEWVAVIWYDGSNKY | |
| CD28 binder: scFv Vh-linker-Vl | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder: R19S mutant | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-64 LC | EVQLVESGGGLVQPGNSLRLSCAASGFTFSSFGMS | 4424 |
| Album binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vh-linker-Vl | SSQGTLVTVSSGGGGSGGGSGGMDWCPRDLWVH | |
| PSMA Mask: Peptide-274 | CFAGSSGGSGGLSGRSDAGSPLGLAGSGGGSQVQ | |
| PSMA binder: wildtype | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-64 HC | GGITPVCKPLWMCSGLGGGSGGLSGRSDAGSPLG | 4425 |
| Album binder: HE-4 | LAGSGGSQVQLVESGGGVVQPGRSLRLSCAASGF | |
| CD28 Mask: Peptide-41 | AFSRYGMHWVRQAPGKGLEWVAVIWYDGSNKY | |
| CD28 binder: scFv Vh-linker-Vl | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder: wildtype | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-65 LC | EVQLVESGGGLVQPGNSLRLSCAASGFTFSSFGMS | 4426 |
| Album binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vh-linker-Vl | SSQGTLVTVSSGGGGSGGGSGGMDWCPRDLWVH | |
| PSMA Mask: Peptide-274 | CFAGSSGGSGGLSGRSDAGSPLGLAGSGGGSQVQ | |
| PSMA binder: R19S mutant | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSGGGGSGGGGSDIQ | |
| | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-65 HC | GGITPVCKPLWMCSGLGGGSGGLSGRSDAGSPLG | 4427 |
| Album binder: HE-4 | LAGSGGSQVQLVESGGGVVQPGRSLSLSCAASGF | |
| CD28 Mask: Peptide-41 | AFSRYGMHWVRQAPGKGLEWVAVIWYDGSNKY | |
| CD28 binder: scFv Vh-linker-Vl | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder; R19S mutant | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-66 LC | EVQLVESGGGLVQPGGSLRLSCAASGSTFYTAVM | 4428 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA Mask: Peptide-274 | GGMDWCPRDLWVHCFAGSSGGSGGLSGRSDAGS | |
| PSMA binder: wildtype | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-66 HC | GGITPVCKPLWMCSGLGGGSGGLSGRSDAGSPLG | 4429 |
| Album binder: HE-1 | LAGSGGSQVQLVESGGGVVQPGRSLRLSCAASGF | |
| CD28 Mask: Peptide-41 | AFSRYGMHWVRQAPGKGLEWVAVIWYDGSNKY | |
| CD28 binder: scFv Vl-linker-Vh | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder: wildtype | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-67 LC | EVQLVESGGGLVQPGGSLRLSCAASGSTFYTAVM | 4430 |
| Album binder: HE-1 | GWVRQAPGKGLEWVAAIRWTALTTSYADSVKG | |
| CD28 Mask: Peptide-41 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA Mask: Peptide-274 | GGMDWCPRDLWVHCFAGSSGGSGGLSGRSDAGS | |
| PSMA binder: R19S mutant | PLGLAGSGGGSDIQMTQSPSSLSASVGDRVTITCH | |
| | ASQNIYVWLNWYQQKPGKAPKLLIYKASNLHTG | |
| | VPSRFSGSGSGTDFTLTISSLQPEDFATYYCQQGQ | |
| | TYPYTFGGGTKVEIKGGGGSGGGGSGGGGSQVQ | |
| | LVQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-67 HC | GGITPVCKPLWMCSGLGGGSGGLSGRSDAGSPLG | 4431 |
| Album binder: HE-1 | LAGSGGSQVQLVESGGGVVQPGRSLSLSCAASGF | |
| CD28 Mask: Peptide-41 | AFSRYGMHWVRQAPGKGLEWVAVIWYDGSNKY | |
| CD28 binder: scFv Vl-linker-Vh | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder: R19S mutant | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-68 LC | EVQLVESGGGLVQPGNSLRLSCAASGFTFSSFGMS | 4432 |
| Album binder: HE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-274 | CFAGSSGGSGGLSGRSDAGSPLGLAGSGGGSDIQ | |
| PSMA binder: wildtype | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-68 HC | GGITPVCKPLWMCSGLGGGSGGLSGRSDAGSPLG | 4433 |
| Album binder: HE-4 | LAGSGGSQVQLVESGGGVVQPGRSLRLSCAASGF | |
| CD28 Mask: Peptide-41 | AFSRYGMHWVRQAPGKGLEWVAVIWYDGSNKY | |
| CD28 binder: scFv Vl-linker-Vh | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder: wildtype | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-69 LC | EVQLVESGGGLVQPGNSLRLSCAASGFTFSSFGMS | 4434 |
| Album binder: HIE-4 | WVRQAPGKGLEWVSSISGSGSDTLYADSVKGRFT | |
| CD28 Mask: Peptide-41 | ISRDNAKTTLYLQMNSLRPEDTAVYYCTIGGSLSR | |
| CD28 binder: scFv Vl-linker-Vh | SSQGTLVTVSSGGGGSGGGSGGMDWCPRDLWVH | |
| PSMA Mask: Peptide-274 | CFAGSSGGSGGLSGRSDAGSPLGLAGSGGGSDIQ | |
| PSMA binder: R19S mutant | MTQSPSSLSASVGDRVTITCHASQNIYVWLNWYQ | |
| | QKPGKAPKLLIYKASNLHTGVPSRFSGSGSGTDFT | |
| | LTISSLQPEDFATYYCQQGQTYPYTFGGGTKVEIK | |
| | GGGGSGGGGSGGGGSQVQLVQSGAEVKKPGASV | |
| | KVSCKASGYTFTSYYIHWVRQAPGQGLEWIGSIY | |
| | PGNVNTNYNEKFKDRATLTVDTSISTAYMELSRL | |
| | RSDDTAVYFCTRSHYGLDWNFDVWGQGTTVTVS | |
| | SGGGGSDIQMTQSPSSLSASVGDRVTITCRASQGIS | |
| | NYLAWYQQKTGKVPKFLIYEASTLQSGVPSRFSG | |
| | GGSGTDFTLTISSLQPEDVATYYCQNYNSAPFTFG | |
| | PGTKVDIKRTVAAPSVFIFPPSDEQLKSGTASVVC | |
| | LLNNFYPREAKVQWKVDNALQSGNSQESVTEQD | |
| | SKDSTYSLSSTLTLSKADYEKHKVYACEVTHQGL | |
| | SSPVTKSFNRGEC | |
| PC-69 HC | GGITPVCKPLWMCSGLGGGSGGLSGRSDAGSPLG | 4435 |
| Album binder: HE-4 | LAGSGGSQVQLVESGGGVVQPGRSLSLSCAASGF | |
| CD28 Mask: Peptide-41 | AFSRYGMHWVRQAPGKGLEWVAVIWYDGSNKY | |
| CD28 binder: scFv Vl-linker-Vh | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder: R19S mutant | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-70 LCAlbum binder: HE-1 | EVQLVESGGGLVQPGGSLRLSCAASGSTFYTAVM | 4436 |
| CD28 Mask: Peptide-127 | GWVRQAPGKGLEWVAAIRWTALTTSYADSVKG | |
| CD28 binder: scFv Vh-linker-Vl | RFTISRDGAKTTLYLQMNSLRPEDTAVYYCAARG | |
| PSMA mask: none | TLGLFTTADSYDYWGQGTLVTVSSGGGGSGGGS | |
| PSMA binder: wildtype | GGYCEGHLPEYLAKCLGSSGGSGGLSGRSDAGSP | |
| | LGLAGSGGGSQVQLVQSGAEVKKPGASVKVSCK | |
| | ASGYTFTSYYIHWVRQAPGQGLEWIGSIYPGNVN | |
| | TNYNEKFKDRATLTVDTSISTAYMELSRLRSDDT | |
| | AVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-70 HC | QVQLVESGGGVVQPGRSLRLSCAASGFAFSRYGM | 4437 |
| Album binder: HE-1 | HWVRQAPGKGLEWVAVIWYDGSNKYYADSVKG | |
| CD28 Mask: Peptide-127 | RFTISRDNSKNTQYLQMNSLRAEDTAVYYCARGG | |
| CD28 binder: scFv Vh-linker-Vl | DFLYYYYYGMDVWGQGTTVTVSSASTKGPSVFP | |
| PSMA mask: none | LAPSSKSTSGGTAALGCLVKDYFPEPVTVSWNSG | |
| PSMA binder: wildtype | ALTSGVHTFPAVLQSSGLYSLSSVVTVPSSSLGTQ | |
| | TYICNVNHKPSNTKVDKKVEPKSC | |
| PC-71 LC | EVQLVESGGGLVQPGGSLRLSCAASGSTFYTAVM | 4438 |
| Album binder: HE-1 | GWVRQAPGKGLEWVAAIRWTALTTSYADSVKG | |
| CD28 Mask: Peptide-127 | RFTISRDGAKTTLYLQMNSLRPEDTAVYYCAARG | |
| CD28 binder: scFv Vh-linker-Vl | TLGLFTTADSYDYWGQGTLVTVSSGGGGSGGGS | |
| PSMA Mask: Peptide-274 | GGYCEGHLPEYLAKCLGSSGGSGGLSGRSDAGSP | |
| PSMA binder: wildtype | LGLAGSGGGSQVQLVQSGAEVKKPGASVKVSCK | |
| | ASGYTFTSYYIHWVRQAPGQGLEWIGSIYPGNVN | |
| | TNYNEKFKDRATLTVDTSISTAYMELSRLRSDDT | |
| | AVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGGGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-71 HC | GGITPVCKPLWMCSGLGGGSGGLSGRSDAGSPLG | 4439 |
| Album binder: HE-1 | LAGSGGSQVQLVESGGGVVQPGRSLRLSCAASGF | |
| CD28 Mask: Peptide-127 | AFSRYGMHWVRQAPGKGLEWVAVIWYDGSNKY | |
| CD28 binder: scFv Vh-linker-Vl | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder: wildtype | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-72 LC | EVQLVESGGGLVQPGGSLRLSCAASGSTFYTAVM | 4440 |
| Album binder: HE-1 | GWVRQAPGKGLEWVAAIRWTALTTSYADSVKG | |
| CD28 Mask: Peptide-127 | RFTISRDGAKTTLYLQMNSLRPEDTAVYYCAARG | |
| CD28 binder: scFv disulfide Vh- | TLGLFTTADSYDYWGQGTLVTVSSGGGGSGGGS | |
| linker-Vl | GGYCEGHLPEYLAKCLGSSGGSGGLSGRSDAGSP | |
| PSMA Mask: Peptide-274 | LGLAGSGGGSQVQLVQSGAEVKKPGASVKVSCK | |
| PSMA binder: wildtype | ASGYTFTSYYIHWVRQAPGQCLEWIGSIYPGNVN | |
| | TNYNEKFKDRATLTVDTSISTAYMELSRLRSDDT | |
| | AVYFCTRSHYGLDWNFDVWGQGTTVTVSSGGG | |
| | GSGGGGSGGGGSDIQMTQSPSSLSASVGDRVTITC | |
| | HASQNIYVWLNWYQQKPGKAPKLLIYKASNLHT | |
| | GVPSRFSGSGSGTDFTLTISSLQPEDFATYYCQQG | |
| | QTYPYTFGCGTKVEIKGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-72 HC | GGITPVCKPLWMCSGLGGGSGGLSGRSDAGSPLG | 4441 |
| Album binder: HE-1 | LAGSGGSQVQLVESGGGVVQPGRSLRLSCAASGF | |
| CD28 Mask: Peptide-127 | AFSRYGMHWVRQAPGKGLEWVAVIWYDGSNKY | |
| CD28 binder: scFv disulfide Vh- | YADSVKGRFTISRDNSKNTQYLQMNSLRAEDTAV | |
| linker-Vl | YYCARGGDFLYYYYYGMDVWGQGTTVTVSSAS | |
| PSMA Mask: Peptide-274 | TKGPSVFPLAPSSKSTSGGTAALGCLVKDYFPEPV | |
| PSMA binder: wildtype | TVSWNSGALTSGVHTFPAVLQSSGLYSLSSVVTV | |
| | PSSSLGTQTYICNVNHKPSNTKVDKKVEPKSC | |
| PC-73 LC | EVQLVESGGGLVQPGGSLRLSCAASGSTFYTAVM | 4442 |
| Album binder: HE-1 | GWVRQAPGKGLEWVAAIRWTALTTSYADSVKG | |
| CD28 Mask: Peptide-127 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA mask: none | GGYCEGHLPEYLAKCLGSSGGSGGLSGRSDAGSP | |
| PSMA binder: wildtype | LGLAGSGGGSDIQMTQSPSSLSASVGDRVTITCHA | |
| | SQNIYVWLNWYQQKPGKAPKLLIYKASNLHTGV | |
| | PSRFSGSGSGTDFTLTISSLQPEDFATYYCQQGQT | |
| | YPYTFGGGTKVEIKGGGGSGGGGGGGGSQVQL | |
| | VQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-73 HC | QVQLVESGGGVVQPGRSLRLSCAASGFAFSRYGM | 4443 |
| Album binder: HE-1 | HWVRQAPGKGLEWVAVIWYDGSNKYYADSVKG | |
| CD28 Mask: Peptide-127 | RFTISRDNSKNTQYLQMNSLRAEDTAVYYCARGG | |
| CD28 binder: scFv Vl-linker-Vh | DFLYYYYYGMDVWGQGTTVTVSSASTKGPSVFP | |
| PSMA mask: none | LAPSSKSTSGGTAALGCLVKDYFPEPVTVSWNSG | |
| PSMA binder: wildtype | ALTSGVHTFPAVLQSSGLYSLSSVVTVPSSSLGTQ | |
| | TYICNVNHKPSNTKVDKKVEPKSC | |
| PC-74 LC | EVQLVESGGGLVQPGGSLRLSCAASGSTFYTAVM | 4444 |
| Album binder: HE-1 | GWVRQAPGKGLEWVAAIRWTALTTSYADSVKG | |
| CD28 Mask: Peptide-127 | RFTISRDGAKTTLYLQMNSLRPEDTAVYYCAARG | |
| CD28 binder: scFv Vl-linker-Vh | TLGLFTTADSYDYWGQGTLVTVSSGGGGSGGGS | |
| PSMA Mask: Peptide-274 | GGYCEGHLPEYLAKCLGSSGGSGGLSGRSDAGSP | |
| PSMA binder: wildtype | LGLAGSGGGSDIQMTQSPSSLSASVGDRVTITCHA | |
| | SQNIYVWLNWYQQKPGKAPKLLIYKASNLHTGV | |
| | PSRFSGSGSGTDFTLTISSLQPEDFATYYCQQGQT | |
| | YPYTFGGGTKVEIKGGGGSGGGGSGGGGSQVQL | |
| | VQSGAEVKKPGASVKVSCKASGYTFTSYYIHWV | |
| | RQAPGQGLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-74 HC | GGITPVCKPLWMCSGLGGGSGGLSGRSDAGSPLG | 4445 |
| Album binder: HE-1 | LAGSGGSQVQLVESGGGVVQPGRSLRLSCAASGF | |
| CD28 Mask: Peptide-127 | AFSRYGMHWVRQAPGKGLEWVAVIWYDGSNKY | |
| CD28 binder: scFv Vl-linker-Vh | YADSVKGRFTISRDNSKNTQYLQMNSLRAEDTAV | |
| PSMA Mask: Peptide-274 | YYCARGGDFLYYYYYGMDVWGQGTTVTVSSAS | |
| PSMA binder: wildtype | TKGPSVFPLAPSSKSTSGGTAALGCLVKDYFPEPV | |
| | TVSWNSGALTSGVHTFPAVLQSSGLYSLSSVVTV | |
| | PSSSLGTQTYICNVNHKPSNTKVDKKVEPKSC | |
| PC-75 LC | EVQLVESGGGLVQPGGSLRLSCAASGSTFYTAVM | 4446 |
| Album binder: HE-1 | GWVRQAPGKGLEWVAAIRWTALTTSYADSVKG | |
| CD28 Mask: Peptide-127 | RFTISRDGAKTTLYLQMNSLRPEDTAVYYCAARG | |
| CD28 binder: scFv disulfide Vl- | TLGLFTTADSYDYWGQGTLVTVSSGGGGSGGGS | |
| linker-Vh | GGYCEGHLPEYLAKCLGSSGGSGGLSGRSDAGSP | |
| PSMA Mask: Peptide-274 | LGLAGSGGGSDIQMTQSPSSLSASVGDRVTITCHA | |
| PSMA binder: wildtype | SQNIYVWLNWYQQKPGKAPKLLIYKASNLHTGV | |
| | PSRFSGSGSGTDFTLTISSLQPEDFATYYCQQGQT | |
| | YPYTFGCGTKVEIKGGGGSGGGGSGGGGSQVQL | |
| | VQSGAEVKKPGASVKVSCKASGYTFTSYYIHWV | |
| | RQAPGQCLEWIGSIYPGNVNTNYNEKFKDRATLT | |
| | VDTSISTAYMELSRLRSDDTAVYFCTRSHYGLDW | |
| | NFDVWGQGTTVTVSSGGGGSDIQMTQSPSSLSAS | |
| | VGDRVTITCRASQGISNYLAWYQQKTGKVPKFLI | |
| | YEASTLQSGVPSRFSGGGSGTDFTLTISSLQPEDVA | |
| | TYYCQNYNSAPFTFGPGTKVDIKRTVAAPSVFIFP | |
| | PSDEQLKSGTASVVCLLNNFYPREAKVQWKVDN | |
| | ALQSGNSQESVTEQDSKDSTYSLSSTLTLSKADYE | |
| | KHKVYACEVTHQGLSSPVTKSFNRGEC | |
| PC-75 HC | GGITPVCKPLWMCSGLGGGSGGLSGRSDAGSPLG | 4447 |
| Album binder: HE-1 | LAGSGGSQVQLVESGGGVVQPGRSLRLSCAASGF | |
| CD28 Mask: Peptide-127 | AFSRYGMHWVRQAPGKGLEWVAVIWYDGSNKY | |
| CD28 binder: scFv disulfide Vl- | YADSVKGRFTISRDNSKNTQYLQMNSLRAEDTAV | |
| linker-Vh | YYCARGGDFLYYYYYGMDVWGQGTTVTVSSAS | |
| PSMA Mask: Peptide-274 | TKGPSVFPLAPSSKSTSGGTAALGCLVKDYFPEPV | |
| PSMA binder: wildtype | TVSWNSGALTSGVHTFPAVLQSSGLYSLSSVVTV | |
| | PSSSLGTQTYICNVNHKPSNTKVDKKVEPKSC | |

L₁ₐ-P₁ₐ-H₁ₐ

Disclosed herein, in some embodiments, are isolated polypeptide or polypeptide complexes according to Formula II: L₁ₐ-P₁ₐ-H₁ₐ wherein U. comprises a tumor specific protease-cleaved linking moiety that when uncleaved connects P₁ₐ to an anti-CD28 antibody and the anti-CD28 antibody is connected to an anti-PSMA antibody, and P₁ₐ comprises a peptide that impairs binding of the anti-CD28 antibody to CD28, and H₁ₐ comprises a half-life extending molecule. In some embodiments. P₁ₐ when L₁ₐ is uncleaved impairs binding of the anti-CD28 antibody to CD28. In some embodiments, the anti-CD28 antibody comprises a human or humanized antibody.

In some embodiments, H₁ₐ comprises a polymer. In some embodiments, the polymer is polyethylene glycol (PEG). In some embodiments, H₁ₐ comprises albumin. In some embodiments. H₁ₐ comprises an Fc domain. In some embodiments, the albumin is serum albumin. In some embodiments, the albumin is human scrum albumin. In some embodiments, H₁ₐ comprises a polypeptide, a ligand, or a small molecule. In some embodiments, the polypeptide, the ligand or the small molecule binds a serum protein or a fragment thereof, a circulating immunoglobulin or a fragment thereof, or CD35/CR 1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, sIgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the antibody comprises a single domain antibody that binds to albumin. In some embodiments, the antibody is a human or humanized antibody. In some embodiments, the single domain antibody is 645gH1gL 1. In some embodiments, the single domain antibody is 645dsgH5gL4. In some embodiments, the single domain antibody is 23-13-A01-sc02. In some embodiments, the single domain antibody is A10m3 or a fragment thereof. In some embodiments, the single domain antibody is DOM7r-31. In some embodiments, the single domain antibody is DOM7 h-11-15. In some embodiments, the single domain antibody is Alb-1, Alb-8, or Alb-23. In some embodiments, the single domain antibody is 10E.

In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 142, HC-CDR2: SEQ ID NO: 143, and HC-CDR3: SEQ ID NO: 144. In some embodiments, the single domain antibody comprises an amino acid sequence with at least 80%, 85%, 900, 95%, 99%, or 100% identity to SEQ ID NO: 141. In some embodiments, the single domain antibody comprises an amino acid sequence with at least 80%, 85%, 90%, 95%, 99%, or 100% identity to SEQ ID NO: 146. In some embodiments, the single domain antibody comprises an amino acid sequence with at least 80%, 85%, 90%, 95%, 99%, or 100% identity to SEQ ID NO: 145. In some embodiments, the single domain antibody comprises an amino acid sequence with at least 80%, 85%, 90%, 95%, 99%, or 100% identity to SEQ ID NO: 4448. In some embodiments, the single domain antibody is SA21.

In some embodiments, H₁, comprises a linking moiety (L₂ₐ) that connects H₁ₐ to P₁ₐ. In some embodiments, L₂ₐ is a peptide sequence having at least 5 to no more than 50 amino acids. In some embodiments, L₂ₐ is a peptide sequence having at least 10 to no more than 30 amino acids. In some embodiments, L₂ₐ is a peptide sequence having at least 10 amino acids. In some embodiments, L₂ₐ is a peptide sequence having at least 18 amino acids. In some embodiments, L₂ₐ is a peptide sequence having at least 26 amino acids. In some embodiments, L₂ₐ has a formula selected from the group consisting of (G₂S)ₙ, (GS)ₙ, (GSGGS)ₙ(SEQ ID NO: 606), (GGGS)ₙ(SEQ ID NO: 607).(GGGGS)ₙ(SEQ ID NO: 608), and (GSSGGS), (SEQ ID NO: 609), wherein n is an integer of at least 1. In some embodiments, L₂. comprises an amino acid sequence according to SEQ ID NO: 110.

In some embodiments, P₁ₐ has less than 70% sequence homology to CD28. In some embodiments, P₁ₐ comprises a peptide sequence of at least 10 amino acids in length. In some embodiments, P₁ₐ comprises a peptide sequence of at least 10 amino acids in length and no more than 20 amino acids in length. In some embodiments, P₁ₐ comprises a peptide sequence of at least 16 amino acids in length. In some embodiments, P₁ₐ comprises a peptide sequence of no more than 40 amino acids in length. In some embodiments, P₁, comprises at least two cysteine amino acid residues. In some embodiments, P₁ₐ comprises a cyclic peptide or a linear peptide. In some embodiments, P₁ₐ comprises a cyclic peptide. In some embodiments, P₁, comprises a linear peptide. In some embodiments, P₁ₐ comprises an amino acid sequence according to any one of SEQ ID NOs: 33 to 106, or 732-813. In some embodiments, P₁ₐ comprises the amino acid sequence of SEQ ID NO: 73. In some embodiments. P₁ₐ comprises the amino acid sequence of SEQ ID NO: 784. In some embodiments, P₁ₐ comprises the amino acid sequence of SEQ ID NO: 73 or a peptide sequence that has 1, 2, or 3 amino acid substitutions, additions, or deletions relative to the amino acid sequence of SEQ ID NO: 73. In some embodiments, P₁ₐ comprises the amino acid sequence of SEQ ID NO: 73. In some embodiments, P₁ₐ comprises the amino acid sequence of SEQ ID NO: 103 or a peptide sequence that has 1, 2, or 3 amino acid substitutions, additions, or deletions relative to the amino acid sequence of SEQ ID NO: 103. In some embodiments, P₁ₐ comprises the amino acid sequence of SEQ ID NO: 103.

In some embodiments, P₁ₐ comprises an amino acid sequence according to X₁-X₂-X₃-C-X₄-X₅-X₆ X₇-X₈-X₉-X₁₀-C-X₁I-X₁₂ wherein X₁ is selected from M, I, L, and V; X₂ is selected from D, H, N, A, F, S, T, Y, and V: X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V: X₇ is selected from L, M, R, S, Q, and H; X₈ is selected from W and Q; X₀ is selected from H, N, D, A, S, Y, T, F, V, L, and I; X₁o is selected from E. V. L, D, Y, R, Q, H, F, K, A, M, and N; X₁₁ is selected from F, Y, L, W, and V; and X₁₂ is selected from N, A, F, S, Y, H, D, T, and L. In some embodiments, X₁ is selected from M, I, and L; X₂ is selected from D, H, N, and A; X₃ is W: X₄ is P; X₅ is selected from R, T, I, M, S, and K; X₆ is selected from E, D, Y, H, S, and F: X₁ is selected from L, M, and R; X₈ is W; X₉ is selected from H, N, D, A, S, and V; X₁₀ is selected from E, V, L, D, and H: X₁₁ is selected from F, Y, and L; and X₁₂ is selected from N, A, F, S, and Y. In some embodiments, X₁ is M; X₂ is selected from D and H; X₃ is W: X₄ is P; X₅ is selected from R, T, and I; X₆ is selected from E, D, and Y: X₇ is selected from L, M, and R. X₈ is W: X₈ is selected from H, N, D, and V; X₁₀ is selected from E, V, L, D, and H: X₁₁ is F; and X₁₂ is selected from N, A, and F. In some embodiments, P₁ₐ comprises an amino acid sequence according to SEQ ID NO: 41 or an amino acid sequence that has 1, 2, or 3 amino acid mutations, substitutions, or deletions relative to SEQ ID NO: 41. In some embodiments, P₁ₐ comprises an amino acid sequence according to SEQ ID NO: 41. In some embodiments, P₁ₐ comprises an amino acid sequence selected from any one of SEQ ID NOs: 41, 73, 103, and 179-603.

In some embodiments. P₁ₐ comprises an amino acid sequence according to: J₁-J₂-W-C-J₃-J₄-J₅-J₆-J₇-J₈-J₉-C-J₁₀-J₁₁, wherein J₁ is selected from A, R, N, D, Q, E, G, H, I, L, K, F, P, S, T, W, Y, and V; J₂ is selected from D, P, and Y: J₃ is selected from P and Q; J₄ is selected from R, L, and I; J₄ is selected from R, L, and I; J₅ is selected from D, H, Y, and I; J₅ is selected from D, H, Y, and I: J₆ is selected from L, S, and R; J₁ is selected from W and G; J₅ is selected from V, A, D, H, L, and N; J₉ is selected from H, L, E, and D; J₁₀ is selected from F, Y, and L; and J₁₁ is selected from A, N, H, and T. In some embodiments, J₁ is selected from A, R, N, D, Q, E, G, H, K, F, P, S, T, W, and Y. In some embodiments. J₁ is selected from R, Q, E, H, I, L, K, F, T, W, Y, and V; J₂ is D; J₃ is P; J₃ is P; J₄ is R: J₅ is D; J is L; J₇ is W; J is V; J₈ is H; J₁₁ is F; and J₁₁ is A. In some embodiments, J₁ is selected from R, Q, E, L, K, T, and W; J₂ is D; J₃ is P; J₄ is R; J₅ is D; J₆ is L; J₇ is W: J₅ is V; J₉ is H; J₁₀ is F; and J₁₁ is A. In some embodiments, J₁ is L or I; J₂ is D or Y: J₃ is P: J₄ is R, L₁ or I: J₅ H, Y, or D: J₁₀ is L or R; J is W: J_{R} is A, D, H, or N: J is L, E, or D; J₁₀ is F or Y; and J₁₁ is N or H. In some embodiments, J is L or I; J₂ is D or Y; J₃ is P; J₄ is R or I; J₅ is H, Y, or D; J₆ is L or R; J₇ is W: J₅ is A, D, or N; 19 is L, E, or D: J₁₀ is For Y; and J₁₁ is N.

In some embodiments, P₁ₐ comprises an amino acid sequence according to: Z₁-C-Z₂-Z₃-Z₄-Z₈-Z₆-Z₇-Z₈-Z₉-Z₁₀-Z₁₁-C-Z₂, wherein Z₁ is selected from Y, H, and A; Z₂ is selected from D, E, A, Q, S, V, M, H, L, F, T, W, and N; Z₃ is selected from G. E, S, and A; Z₄ is selected from H, L, W, and A; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₁ is selected from E, Q, I, M, V, A, L, R, W, P, D, T, K, S, G, H, and N; Z₈ is selected from Y, H, F, W, N, Q, and A; Z₉ is selected from M, I, L, R, V, K, F, Q, Y, W, and A; Z₁₀ is selected from A, Q, S, W, E, L, G, D, T, M, N, V, H, R, Y, K, P, F, and I; Z₁₁ is selected from K, Q, N, H, E, P, and A; and Z₂ is selected from L, M, I, F, V, Y, Q, D, T, and A. In some embodiments, Z₁ is selected from Y and H: Z₂ is selected from D, E, A, Q, S, V, M, H, L, F, T, W, and N; Z₃ is selected from G, E, and S; Z₄ is selected from H, L, and W; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y; Z₇ is selected from E, Q, I, M, V, A, L, R, W, P, D, T, K, S, G, H, and N; Z₈ is selected from Y, H, F, W, N, and Q; Z₉ is selected from M, I, L, R, V, K, F, Q, Y, and W: Z₁₀ is selected from A, Q, S, W, E, L, G, D, T, M, N, V, H, R, Y, K, P, F, and I; Z₁₁ is selected from K, Q, N, H, E, and P; and Z₂ is selected from L, M, I, F, V, Y, Q, D, and T. In some embodiments. Z₁ is selected from Y and H; Z₂ is selected from D, E, A, Q, S, V, M, H, L, F, T, W, and N; Z₃ is selected from G, E, and S: Z₄ is selected from H, L, and W; Z₅ is selected from L, S, A, T, I, V, M, R, E, D, F, H, K, Q, N, Y, W, and G; Z₆ is selected from P, A, E, Q, S, L, W, G, V, D, K, I, M, R, T, F, H, N, and Y: Z₇ is selected from E, Q, I, M, V, A, L, R, W, P, D, T, K, S, G, H, and N: Z₈ is selected from Y, H, F, W, N, and Q; Z₉ is selected from M, I, L, R, V, K, F, Q, Y, and W: Z₁₀ is selected from A, Q, S, W, E, L, G, D, T, M, N, V, H, R, Y, K, P, F, and I; Z₁₁ is selected from K, Q, N, H, E, and P; and Z₁₂ is selected from L, M, I, F, V, Y, Q, D, and T. In some embodiments, Z₁ is Y; Z₂ is selected from D. E, A, and Q; Z₃ is G; Z₄ is H; Z₈ is selected from L, S, A, T, I, V, and M: Z₆ is selected from P, A, E, Q, S, L, W, G, V, and D; Z₁ is selected from E, Q, I, M, V, A, and L; Z₈ is selected from Y and H; Z₉ is selected from M, I, and L: Z₁o is selected from A, Q, S, W, E, L, G, D, T, M, and N; Z₁₁ is K; and Z₁₂ is selected from L and M. In some embodiments, Z₁ is Y; Z₂ is selected from D, E, and A; Z₃ is G; Z₁, is H; Z₄ is H; Z₅ is selected from L, S, A, and T; Z₆ is selected from P, A, E, Q, S, L, and W: Z₁ is selected from E, Q, I, and M; Z₈ is Y; Z₉ is selected from M, I, and L: Z₁₀ is selected from A, Q, S, W, E, L, G, and D; Z₁ is K; and Z₁ is L. In some embodiments, Z₁ is Y; Z₂ is selected from D and E; Z₃ is G; Z₄ is H; Z₅ is selected from L, S, and A; Z₆ is selected from P, A, E, Q, S, and L; Z₁ is selected from E, Q, and I; Z₈ is Y; Z₁₁ is selected from M, I, and L; Z₁₀ is selected from A, Q, S, W, E, L, G, and D; Z₁₁ is K; and Z₁₂ is L.

In some embodiments, P₁ₐ comprises the amino acid sequence of SEQ ID NO: 784. In some embodiments, P₁ₐ consists of the amino acid sequence of SEQ ID NO: 784. In some embodiments, P₁ₐ comprises the amino acid sequence of SEQ ID NO: 744. In some embodiments, P₁ₐ consists of the amino acid sequence of SEQ ID NO: 744. In some embodiments, P₁ₐ comprises an amino acid sequence according to any one of SEQ ID NOs: 774-793. In some embodiments. P₁₈ comprises an amino acid sequence according to any one of SEQ ID NOs: 764-773.

In some embodiments, P₁ₐ comprises an amino acid sequence according to: U₁-C-U₂-U₃-U₄-U₅-U₆-U₇-U₈-U₉-U₁₀-U₁₁-C-U₁₂, wherein: U₁ is selected from T, S, L, M, W, Q, P, R, G, N, I, Y, D, E, V, H, and A; U₂ is selected from A. S, T, K, E, R, G, N, W, and D; U₃ is selected from A, E, P, D, G, W, Q, V, and F; U₄ is selected from G, V, F, D, E, R, and A; U₅ is selected from A, F, V, S, L, P, and M: U₆ is selected from T, S, H, M, A, N, P, G, Q, D, Y, V, L, I, E, K, F, and R U₇ is selected from Y. W, F, L, N, T, Q, I, and A; U₈ is selected from P, S, T, A, E, K, Q, N, V, G, H, L, D, Y, I, M, and R; U₉ is selected from W, L, and A; U₁₀ is selected from P, S, R, L, E, and A; U₁₁ is selected from L, A, T, M, V, W, Y, E, Q, I, F, S, P, K, R, D, H, N, and G; and U₁₂ is selected from L, F, V, M, I, Y, E, W, and A. In some embodiments, U₁ is selected from T, S, L, M, W, Q, P, R, G, N, I, Y, D, E, V, and H; U₂ is selected from A, S, T, K, E, R, G, N, W, and D; U₃ is selected from A, E, P, D, G, W, Q, V, and F: U₄ is selected from G, V, F, D, E, and R: U₅ is selected from A, F, V, S, L, P, and M; U₆ is selected from T, S, H, M, A, N, P, G, Q, D, Y, V, L, I, E, K, F, and R: U₇ is selected from Y, W, F, L, N, T, Q, and I; U₈ is selected from P, S, T, A, E, K, Q, N, V, G, H, L, D, Y, I, M, and R; U₉ is selected from W, L, and A: U₁₀ is selected from P, S, R, L, and E; U₁ is selected from L, A, T, M, V, W, Y, E, Q, I, F, S, P, K, R, D, H, N, and G; and U₁ is selected from L, F, V, M, I, Y, E, and W. In some embodiments, U₁ is selected from T, S, L, M, and W: U₂ is selected from A, S, and T: U₃ is selected from A and E: U₄ is selected from G, V, and F: U₈ is selected from A. F, and V; U₆ is selected from T, S, H, M, A, and N; U₇ is selected from Y, W, and F; U₈ is selected from P, S, T, A, E, K, Q, N, and V; U₉ is selected from W and L; U₁₀ is selected from P and S: U₁₁ is selected from L, A, T, M, V, W, Y, E, Q, I, F, and S; and U₁₂ is selected from L, F, V, and M. In some embodiments, U₁ is selected from T and S; U₂ is selected from A and S; U₃ is A; U₄ is selected from G and V; U₈ is selected from A and F; U₆ is selected from T, S, and H; U₇ is selected from Y and W; U₈ is selected from P, S, T, and A; U₉ is W; U₁₀ is P; U₁₁ is selected from L, A, T, M, V, W, and Y; and U₁₂ is selected from L and F.

In some embodiments, P₁ₐ comprises the amino acid sequence of SEQ ID NO: 749. In some embodiments, P₁ₐ comprises the amino acid sequence of SEQ ID NO: 749. In some embodiments, P₁ₐ comprises an amino acid sequence according to any one of SEQ ID NOs: 756-763. In some embodiments, P₁ₐ comprises an amino acid sequence according to any one of SEQ ID NOs: 794-813. In some embodiments, P₁ₐ comprises an amino acid sequence according to any one of SEQ ID NOs: 732-2002.

### Modified Amino Acids

In some embodiments, the isolated polypeptide or polypeptide complex comprises a modified amino acid, a non-natural amino acid, a modified non-natural amino acid, or a combination thereof. In some embodiments, the non-natural amino acid comprises a D-amino acid. In some embodiments, the modified amino acid or modified non-natural amino acid comprises a post-translational modification.

### Pharmaceutical Compositions

Disclosed herein, in some embodiments, are pharmaceutical compositions comprising an isolated polypeptide or polypeptide complex of any one of the embodiments disclosed herein.

Disclosed herein, in some embodiments, are pharmaceutical compositions comprising an isolated polypeptide or polypeptide complex that comprises a first antigen recognizing molecule that binds to CD28 and a second antigen recognizing molecule that binds to prostate-specific membrane antigen. In some embodiments, the first antigen recognizing molecule that binds to CD28 comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6. In some embodiments, the anti-CD28 heavy chain variable domain comprises an amino acid sequence with at least 80%, 85%, 90%, 95%, 98%, 99%, or 100% sequence identity to SEQ ID NO: 7, and wherein the anti-CD28 light chain variable domain comprises an amino acid sequence with at least 80%, 85%, 90%, 95%, 98%, 99%, or 100% sequence identity to SEQ ID NO: 8. In some embodiments, the anti-CD28 antibody comprises a single chain variable fragment (scFv), a single domain antibody, a Fab, a Fab, or a CrossFab. In some embodiments, the anti-CD28 antibody comprises the scFv. In some embodiments, the anti-CD28 antibody comprises a single chain variable fragment (scFv) with an engineered disulfide. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 9. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 10. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 11. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 12. In some embodiments, the anti-CD28 antibody comprises the Fab. In some embodiments, the Fab comprises an amino acid sequence with at least 80%, 85%, 90%, 95%, 98%, 99%, or 100% sequence identity to SEQ ID NO: 696 and an amino acid sequence with at least 80%, 85%, 90%, 95%, 98%, 99%, or 100% sequence identity to SEQ ID NO: 697. In some embodiments, the anti-CD28 antibody comprises the CrossFab. In some embodiments, the CrossFab comprises an amino acid sequence with at least 80%, 85%, 90%, 95%, 98%, 99%, or 100% sequence identity to SEQ ID NO: 698 and an amino acid sequence with at least 80%, 85%, 90%, 95%, 98%, 99%, or 100% sequence identity to SEQ ID NO: 699. In some embodiments, the CrossFab comprises an amino acid sequence with at least 80%, 85%, 90%, 95%, 98%, 99%, or 100% sequence identity to SEQ ID NO: 700 and an amino acid sequence with at least 80%, 85%, 90%, 95%, 98%, 99%, or 100% sequence identity to SEQ ID NO: 701.

In some embodiments, the second antigen recognizing molecule that binds to PSMA comprises an anti-PSMA antibody. In some embodiments, the anti-PSMA antibody comprises an anti-PSMA heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 13, HC-CDR2: SEQ ID NO: 14, and HC-CDR3: SEQ ID NO: 15 and the anti-PSMA antibody comprises an anti-PSMA light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprise an amino acid sequence of LC-CDR1: SEQ ID NO: 16, LC-CDR2: (EA), and LC-CDR3: SEQ ID NO: 18. In some embodiments, the anti-PSMA heavy chain variable domain comprises an amino acid sequence with at least 80%, 85%, 90%, 95%, 98%, 99%, or 100% sequence identity to SEQ ID NO: 20, and the anti-PSMA light chain variable domain comprises an amino acid sequence with at least 80%, 85%, 90%, 95%, 98%, 99%, or 100% sequence identity to SEQ ID NO: 19. In some embodiments, the anti-PSMA antibody comprises a single chain variable fragment (scFv), a single domain antibody, a Fab, a Fab′, or a CrossFab. In some embodiments, the anti-PSMA antibody comprises the Fab or Fab′. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 19 and an amino acid sequence with at least at least 80%, 85%, 90%, 95%, 98%, 99%, or 100% sequence identity to SEQ ID NO: 20. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 702 and an amino acid sequence with at least at least 80%, 85%, 90%, 95%, 98%, 99%, or 100% sequence identity to SEQ ID NO: 703. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 704 and an amino acid sequence with at least at least 80%, 85%, 90%, 95%, 98%, 99%, or 100/a sequence identity to SEQ ID NO: 705.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein. may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservatives, emulsifying agents and dispersing agents. Prevention of the action of microorganisms may be ensured by the inclusion of various antibacterial and antifungal agents.

The pharmaceutical composition may be in any suitable form, depending upon the desired method of administration. It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc, and a physician will ultimately determine appropriate dosages to be used.

### Isolated Recombinant Nucleic Acid Molecules

Disclosed herein are isolated recombinant nucleic acid molecules encoding an isolated polypeptide or polypeptide complex of any one of the embodiments disclosed herein.

Disclosed herein, in some embodiments, are isolated recombinant nucleic acid molecules encoding a polypeptide or polypeptide complex that comprises a first antigen recognizing molecule that binds to CD28 and a second antigen recognizing molecule that binds to prostate-specific membrane antigen. In some embodiments, the first antigen recognizing molecule that binds to CD28 comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6. In some embodiments, the anti-CD28 heavy chain variable domain comprises an amino acid sequence with at least 80%, 85%, 90%, 95%, 98%, 99%, or 100% sequence identity to SEQ ID NO: 7, and wherein the anti-CD28 light chain variable domain comprises an amino acid sequence with at least 80%, 85%, 90%, 95%, 98%, 99%, or 100% sequence identity to SEQ ID NO: 8. In some embodiments, the anti-CD28 antibody comprises a single chain variable fragment (scFv), a single domain antibody, a Fab, or a Fab′. In some embodiments, the anti-CD28 antibody comprises the scFv. In some embodiments, the anti-CD28 antibody comprises a single chain variable fragment (scFv) with an engineered disulfide. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 9. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 10. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 11. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 12. In some embodiments, the anti-CD28 antibody comprises the Fab. In some embodiments, the Fab comprises an amino acid sequence with at least 80%, 85%, 90%, 95%, 98%, 99%, or 100% sequence identity to SEQ ID NO: 696 and an amino acid sequence with at least 80%, 85%, 90%, 95%, 98%, 99%, or 100% sequence identity to SEQ ID NO: 697. In some embodiments, the anti-CD28 antibody comprises the CrossFab. In some embodiments, the CrossFab comprises an amino acid sequence with at least 80%, 85%, 90%, 95%, 98%, 99%, or 100% sequence identity to SEQ ID NO: 698 and an amino acid sequence with at least 80%, 85%, 90%, 95%, 98%, 99%, or 100% sequence identity to SEQ ID NO: 699. In some embodiments, the CrossFab comprises an amino acid sequence with at least 80%, 85%, 90%, 95%, 98%, 99%, or 100% sequence identity to SEQ ID NO: 700 and an amino acid sequence with at least 80%, 85%, 90%, 95%, 98%, 99%, or 100% sequence identity to SEQ ID NO: 701.

In some embodiments, the second antigen recognizing molecule that binds to PSMA comprises an anti-PSMA antibody. In some embodiments, the anti-PSMA antibody comprises an anti-PSMA heavy chain variable domain that comprises complementarity determining regions (CDRs): HC-CDR 1, HC-CDR2, and HC-CDR3, wherein the HC-CDR1, the HC-CDR2, and the HC-CDR3 comprise amino acid sequences of HC-CDR1: SEQ ID NO: 13, HC-CDR2: SEQ ID NO: 14, and HC-CDR3: SEQ ID NO: 15 and the anti-PSMA antibody comprises an anti-PSMA light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprise an amino acid sequence of LC-CDR1: SEQ ID NO: 16, LC-CDR2: (EA), and LC-CDR3: SEQ ID NO: 18. In some embodiments, the anti-PSMA heavy chain variable domain comprises an amino acid sequence with at least 80%, 85%, 90%, 95%, 98%, 99%, or 100% sequence identity to SEQ ID NO: 20, and the anti-PSMA light chain variable domain comprises an amino acid sequence with at least 80%, 85%, 90%, 95%, 98%, 99%, or 100% sequence identity to SEQ ID NO: 19. In some embodiments, the anti-PSMA antibody comprises a single chain variable fragment (scFv), a single domain antibody, a Fab, a Fab′, or a CrossFab. In some embodiments, the anti-PSMA antibody comprises the Fab or Fab′. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 19 and an amino acid sequence with at least at least 80%, 85/, 90%, 95%, 98%, 99%, or 100% sequence identity to SEQ ID NO: 20. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 702 and an amino acid sequence with at least at least 80%, 85%, 90%, 95%, 98%, 99%, or 100% sequence identity to SEQ ID NO: 703. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 704 and an amino acid sequence with at least at least 80%, 85%, 90%, 95%, 98%, 99%, or 100% sequence identity to SEQ ID NO: 705.

### Methods of Treatment

Disclosed herein, in some embodiments, are methods of treating cancer in a subject in need thereof comprising administering to the subject an isolated polypeptide or polypeptide complex of any one of the embodiments disclosed herein. In some embodiments, the cancer has cells that express PSMA. In some instances, the cancer is a solid tumor cancer. In some embodiments, the cancer is lung, breast (e.g. HER2+; ER/PR+; TNBC), cervical, ovarian, colorectal, pancreatic or gastric.

In some embodiments, are methods of treating prostate cancer in a subject need in need thereof comprising administering to the subject an isolated polypeptide or polypeptide complex as described herein. In some embodiments, are methods of treating metastatic castrate-resistant prostate cancer (mCRPC) in a subject need in need thereof comprising administering to the subject an isolated polypeptide or polypeptide complex as described herein.

Disclosed herein, in some embodiments, are methods of treating cancer in a subject in need thereof comprising administering to the subject a polypeptide or polypeptide complex that comprises a first antigen recognizing molecule that binds to CD28 and a second antigen recognizing molecule that binds to prostate-specific membrane antigen. In some embodiments, the first antigen recognizing molecule that binds to CD28 comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDRL: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6. In some embodiments, the anti-CD28 heavy chain variable domain comprises an amino acid sequence with at least 80%, 85%, 90%, 95%, 98%, 99%, or 100% sequence identity to SEQ ID NO: 7, and wherein the anti-CD28 light chain variable domain comprises an amino acid sequence with at least 80%, 85%, 90%, 95%, 98%, 99%, or 100% sequence identity to SEQ ID NO: 8. In some embodiments, the anti-CD28 antibody comprises a single chain variable fragment (scFv), a single domain antibody, a Fab, a Fab′, or a CrossFab. In some embodiments, the anti-CD28 antibody comprises the scFv. In some embodiments, the anti-CD28 antibody comprises a single chain variable fragment (scFv) with an engineered disulfide. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 9. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 10. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 11. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 12. In some embodiments, the anti-CD28 antibody comprises the Fab. In some embodiments, the Fab comprises an amino acid sequence with at least 80%, 85%, 90%, 95%, 98%, 99%, or 100% sequence identity to SEQ ID NO: 696 and an amino acid sequence with at least 80%, 85%, 90%, 95%, 98%, 99%, or 100% sequence identity to SEQ ID NO: 697. In some embodiments, the anti-CD28 antibody comprises the CrossFab. In some embodiments, the CrossFab comprises an amino acid sequence with at least 80%, 85%, 90%, 95%, 98%, 99%, or 100% sequence identity to SEQ ID NO: 698 and an amino acid sequence with at least 80%, 85%, 90%, 95%, 98%, 99%, or 100% sequence identity to SEQ ID NO: 699. In some embodiments, the CrossFab comprises an amino acid sequence with at least 80%, 85%, 90,/a 95%, 98%, 99%, or 100% sequence identity to SEQ ID NO: 700 and an amino acid sequence with at least 80%, 85%, 90%, 95%, 98%, 99%, or 100% sequence identity to SEQ ID NO: 701.

In some embodiments, the second antigen recognizing molecule that binds to PSMA comprises an anti-PSMA antibody. In some embodiments, the anti-PSMA antibody comprises an anti-PSMA heavy chain variable domain that comprises complementarity determining regions (CDRs): HC-CDR1, HC-CDR2, and HC-CDR3, wherein the HC-CDR 1, the HC-CDR2, and the HC-CDR3 comprise amino acid sequences of HC-CDRL: SEQ ID NO: 13, HC-CDR2: SEQ ID NO: 14, and HC-CDR3: SEQ ID NO: 15 and the anti-PSMA antibody comprises an anti-PSMA light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprise an amino acid sequence of LC-CDRL: SEQ ID NO: 16, LC-CDR2: (EA), and LC-CDR3: SEQ ID NO: 18. In some embodiments, the anti-PSMA heavy chain variable domain comprises an amino acid sequence with at least 80%, 85%, 90%, 95%, 98%, 99%, or 100% sequence identity to SEQ ID NO: 20, and the anti-PSMA light chain variable domain comprises an amino acid sequence with at least 80%, 85%, 90%, 95%, 98%, 99%, or 100% sequence identity to SEQ ID NO: 19. In some embodiments, the anti-PSMA antibody comprises a single chain variable fragment (scFv), a single domain antibody, a Fab, a Fab′, or a CrossFab. In some embodiments, the anti-PSMA antibody comprises the Fab or Fab′. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 19 and an amino acid sequence with at least at least 80%, 85%, 90%, 95%, 98%, 99%, or 100% sequence identity to SEQ ID NO: 20. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 702 and an amino acid sequence with at least at least 80%, 85%, 90%, 95%, 98%, 99%, or 100% sequence identity to SEQ ID NO: 703. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 704 and an amino acid sequence with at least at least 80%, 85%, 90%, 95%, 98%, 99%, or 100% sequence identity to SEQ ID NO: 705.

### Combination Treatment

In some embodiments, the polypeptide or polypeptide complex induces T cell mediated cytotoxicity of tumor cells. In some embodiments, the cancer has cells that express PSMA. In some embodiments, the cancer is a solid tumor cancer. In some embodiments, the cancer is lung, breast, cervical, ovarian, colorectal, pancreatic or gastric cancer.

In some embodiments, the polypeptide or polypeptide complex is administered in combination with an anti-cancer therapy. In some embodiments, the polypeptide or polypeptide complex and the anti-cancer therapy are administered in the same pharmaceutical composition. In some embodiments, the polypeptide or polypeptide complex and the anti-cancer therapy are administered as separate pharmaceutical compositions. In some embodiments, the anti-cancer therapy comprises a small molecule, a cell-based therapy, or an antibody-based therapy.

In some embodiments, the antibody-based therapy is a T cell engager. In some embodiments, the T cell engager comprises a formula according to: D-L₀-E (Formula II), wherein D comprises an effector cell binding domain that binds to an effector cell antigen, E comprises a tumor antigen binding domain that binds to a tumor antigen, and L>comprises a linker that connects D to E. In some embodiments, D comprises a single chain variable fragment, a single domain antibody, or a Fab fragment. In some embodiments, D comprises the single chain variable fragment. In some embodiments, E comprises a single chain variable fragment, a single domain antibody, or a Fab fragment. In some embodiments, E comprises the Fab fragment.

In some embodiments, the effector cell binding domain comprises complementary determining regions (CDRs) selected from the group consisting of muromonab-CD3 (OKT3), otelixizumab (TRX4), teplizumab (MGA031). visilizumab (Nuvion). SP34, X35, VIT3, BMA030 (BW264/56), CLB-T3/3, CRIS7, YTH12.5, F111-409, CLB-T3.4.2, TR-66,WT32, SPv-T3b, 11ID8, XIII-141, XIII-46, XIII-87, 121F6, T3/RW2-8C8, T3/RW2-4B6, OKT3D, M-T301, SMC2, F101.01, UCHT-1, WT-31, 15865, 15865v12, 15865v 16, and 15865v 19. In some embodiments, the effector cell binding domain comprises an amino acid sequence according to SEQ ID NOs: 611-624.

TABLE 14
Effector cell binding domain amino acid sequences

| | Amino Acid Sequence | SEQ ID |
|---|---|---|
| Construct Description | (N to C) | NO: |
| SP34.185 CD3: HC: CDR1 | GFTFNKYA | 611 |
| SP34.185 CD3: HC: CDR2 | IRSKYNNYAT | 612 |
| SP34.185 CD3: HC: CDR3 | VRHGNFGNSYISYWAY | 613 |
| SP34.185 CD3: LC: CDR1 | TGAVTSGNY | 614 |
| SP34.185 CD3: LC: CDR2 | GTK | |
| SP34.185 CD3: LC: CDR3 | VLWYSNRWV | 616 |
| SP34.194 CD3: HC: CDR1 | GFTFNTYA | 617 |
| SP34.194 CD3: HC: CDR2 | IRSKYNNYAT | 618 |
| SP34.194 CD3: HC: CDR3 | VRHGNFGNSYVSWFAY | 619 |
| SP34.194 CD3: LC: CDR1 | TGAVTTSNY | 620 |
| SP34.194 CD3: LC: CDR2 | GT | |
| SP34.194 CD3: LC: CDR3 | ALWYSNLWV | 622 |
| SP34.185 scFv | EVQLVESGGGLVQPGGSLKLS | 623 |
| (VH - linker 1 - VL) | CAASGFTFNKYAMNWVRQA | |
| | PGKGLEWVARIRSKYNNYAT | |
| | YYADSVKDRFTISRDDSKNTA | |
| | YLQMNNLKTEDTAVYYCVRH | |
| | GNFGNSYISYWAYWGQGTLV | |
| | TVSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKL | |
| | TVL | |
| SP34.194 scFv | QTVVTQEPSLTVSPGGTVTLT | 624 |
| (VL - linker 1 - VH) | CRSSTGAVTTSNYANWVQQK | |
| | PGQAPRGLIGGTNKRAPGTPA | |
| | RFSGSLLGGKAALTLSGVQPE | |
| | DEAEYYCALWYSNLWVFGG | |
| | GTKLTVLGGGGSGGGGSGGG | |
| | GSEVQLVESGGGLVQPGGSLK | |
| | LSCAASGFTFNTYAMNWVRQ | |
| | APGKGLEWVARIRSKYNNYA | |
| | TYYADSVKDRFTISRDDSKNT | |
| | AYLQMNNLKTEDTAVYYCVR | |
| | HGNFGNSYVSWFAYWGQGT | |
| | LVTVSS | |
| SP34.185 Peptide mask (P3) | GSQCLGPEWEVCPY | 625 |
| SP34.185 Peptide mask (P3) | VYCGPEFDESVGCM | 626 |
| SP34.185 Peptide mask (P3) | VYCGPEFDESVGCA | 627 |
| SP34.185 Peptide mask (P3) | YLCGPDGDETLACY | 628 |

In some embodiments, the tumor antigen comprises epidermal growth factor receptor (EGFR), prostate-specific membrane antigen (PSMA), or tumor-associated calcium signal transducer 2 (referred to herein after as TROP2). In some embodiments, the tumor antigen comprises EGFR. In some embodiments, the tumor antigen binding domain comprises an amino acid sequence according to SEQ ID NOs: 629-638. In some embodiments, the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 632; HC-CDR2: SEQ ID NO: 633; HC-CDR3: SEQ ID NO: 634; and wherein the LC-CDR1, the LC-CDR2, and the LC-CDR3 comprise: LC-CDR1: SEQ ID NO: 629; LC-CDR2: (YAS); and LC-CDR3: SEQ ID NO: 631. In some embodiments, the tumor antigen comprises EGFR, and the T cell engager comprises amino acid sequences with at least 95% sequence identity according to SEQ ID NOs: 639 and 640. In some embodiments, the tumor antigen comprises EGFR, and the T cell engager comprises amino acid sequences according to SEQ ID NOs: 641 and 642.

In some embodiments, the tumor antigen comprises TROP2. In some embodiments, the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 659; HC-CDR2: SEQ ID NO: 660; HC-CDR3: SEQ ID NO: 661, 662, or 663; and wherein the LC-CDR1, the LC-CDR2, and the LC-CDR3 comprise: LC-CDR1: SEQ ID NO: 664; LC-CDR2: (SAS); and LC-CDR3: SEQ ID NO: 666. In some embodiments, the tumor antigen comprises TROP2, and the T cell engager comprises amino acid sequences with at least 95% sequence identity according to SEQ ID NOs: 673 and 674. In some embodiments, the tumor antigen comprises TROP2, and the T cell engager comprises amino acid sequences according to SEQ ID NOs: 675 and 676, or SEQ ID NOs: 677 and 678. In some embodiments, the tumor antigen binding domain comprises an amino acid sequence according to SEQ ID NOs: 659-668.

In some embodiments, the tumor antigen comprises PSMA. In some embodiments, the tumor antigen binding domain comprises an amino acid sequence according to SEQ ID NOs: 647-654. In some embodiments, the tumor antigen comprises PSMA, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 647; HC-CDR2: SEQ ID NO: 648; HC-CDR3: SEQ ID NO: 649; and wherein the LC-CDR1, the LC-CDR2, and the LC-CDR3 comprise:LC-CDR1: SEQ ID NO: 650; LC-CDR2: SEQ ID NO: 651; and LC-CDR3: SEQ ID NO: 652. In some embodiments, the tumor antigen comprises PSMA, and the T cell engager comprises amino acid sequences with at least 95% sequence identity according to SEQ ID NOs: 655 and 656.

TABLE 15
Tumor antigen binding domain amino acid sequences - anti-EGFR

| Construct | Amino Acid Sequence | |
|---|---|---|
| Description | (N to C) | SEQ ID NO: |
| EGFR: LC: CDR1 | QSIGTN | 629 |
| EGFR: LC: CDR2 | YAS | |
| EGFR: LC: CDR3 | QQNNNWPTT | 631 |
| EGFR: HC: CDR1 | GFSLTNYG | 632 |
| EGFR: HC: CDR2 | IWSGGNT | 633 |
| EGFR: HC: CDR3 | ARALTYYDYEFAY | 634 |
| EGFR Fab LC v1 | QILLTQSPVILSVSPGERVSFSCRASQ | 635 |
| | SIGTNIHWYQQRTNGSPRLLIKYAS | |
| | ESISGIPSRFSGSGSGTDFTLSINSVES | |
| | EDIADYYCQQNNNWPTTFGAGTKL | |
| | ELKRTVAAPSVFIFPPSDEQLKSGTA | |
| | SVVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR Fab LC v2 | DILLTQSPVILSVSPGERVSFSCRASQ | 636 |
| | SIGTNIHWYQQRINGSPRLLIKYAS | |
| | ESISGIPSRFSGSGSGTDFTLSINSVES | |
| | EDIADYYCQQNNNWPTTFGAGTKL | |
| | ELKRTVAAPSVFIFPPSDEQLKSGTA | |
| | SVVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR Fab HC | QVQLKQSGPGLVQPSQSLSITCTVS | 637 |
| | GFSLTNYGVHWVRQSPGKGLEWL | |
| | GVIWSGGNTDYNTPFTSRLSINKDN | |
| | SKSQVFFKMNSLQSNDTAIYYCARA | |
| | LTYYDYEFAYWGQGTLVTVSAAST | |
| | KGPSVFPLAPSSKSTSGGTAALGCL | |
| | VKDYFPEPVTVSWNSGALTSGVHTF | |
| | PAVLQSSGLYSLSSVVTVPSSSLGTQ | |
| | TYICNVNHKPSNTKVDKKVEPKSC | |
| EGFR Fab HC | QVQLKQSGPGLVQPSQSLSITCTVS | 638 |
| (N88Q) | GFSLTNYGVHWVRQSPGKGLEWL | |
| | GVIWSGGNTDYNTPFTSRLSINKDN | |
| | SKSQVFFKMNSLQSQDTAIYYCARA | |
| | LTYYDYEFAYWGQGTLVTVSAAST | |
| | KGPSVFPLAPSSKSTSGGTAALGCL | |
| | VKDYFPEPVTVSWNSGALTSGVHTF | |
| | PAVLQSSGLYSLSSVVTVPSSSLGTQ | |
| | TYICNVNHKPSNTKVDKKVEPKSC | |
| EGFR TCE-1 LC | QILLTQSPVILSVSPGERVSFSCRASQ | 639 |
| | SIGTNIHWYQQRINGSPRLLIKYASE | |
| | SISGIPSRFSGSGSGTDFTLSINSVESE | |
| | DIADYYCQQNNNWPTTFGAGTKLE | |
| | LKRTVAAPSVFIFPPSDEQLKSGTAS | |
| | VVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR TCE-1 HC | EVQLVESGGGLVQPGGSLKLSCAAS | 640 |
| | GFTFNKYAMNWVRQAPGKGLEWV | |
| | ARIRSKYNNYATYYADSVKDRFTIS | |
| | RDDSKNTAYLQMNNLKTEDTAVY | |
| | YCVRHGNFGNSYISYWAYWGQGTL | |
| | VTVSSGGGGSGGGGSGGGGSQTVV | |
| | TQEPSLTVSPGGTVTLTCGSSTGAV | |
| | TSGNYPNWVQQKPGQAPRGLIGGT | |
| | KFLAPGTPARFSGSLLGGKAALTLS | |
| | GVQPEDEAEYYCVLWYSNRWVFG | |
| | GGTKLTVLGGGGSQVQLKQSGPGL | |
| | VQPSQSLSITCTVSGFSLTNYGVHW | |
| | VRQSPGKGLEWLGVIWSGGNTDYN | |
| | TPFTSRLSINKDNSKSQVFFKMNSLQ | |
| | SQDTAIYYCARALTYYDYEFAYWG | |
| | QGTLVTVSAASTKGPSVFPLAPSSKS | |
| | TSGGTAALGCLVKDYFPEPVTVSW | |
| | NSGALTSGVHTFPAVLQSSGLYSLS | |
| | SVVTVPSSSLGTQTYICNVNHKPSN | |
| | TKVDKKVEPKSC | |
| EGFR TCE-2 LC | QILLTQSPVILSVSPGERVSFSCRASQ | 641 |
| | SIGTNIHWYQQRTNGSPRLLIKYASE | |
| | SISGIPSRFSGSGSGTDFTLSINSVESE | |
| | DIADYYCQQNNNWPTTFGAGTKLE | |
| | LKRTVAAPSVFIFPPSDEQLKSGTAS | |
| | VVCLLNNFYPREAKVQWKVDNAL | |
| | QSGNSQESVTEQDSKDSTYSLSSTLT | |
| | LSKADYEKHKVYACEVTHQGLSSP | |
| | VTKSFNRGEC | |
| EGFR TCE-2 HC | EVQLVESGGGLVQPGGSLKLSCAAS | 642 |
| | GFTFNKYAMNWVRQAPGKGLEWV | |
| | ARIRSKYNNYATYYADSVKDRFTIS | |
| | RDDSKNTAYLQMNNLKTEDTAVY | |
| | YCVRHGNFGNSYISYWAYWGQGTL | |
| | VTVSSGGGGSGGGGSGGGGSQTVV | |
| | TQEPSLTVSPGGTVTLTCGSSTGAV | |
| | TSGNYPNWVQQKPGQAPRGLIGGT | |
| | KFLAPGTPARFSGSLLGGKAALTLS | |
| | GVQPEDEAEYYCVLWYSNRWVFG | |
| | GGTKLTVLGGGGSQVQLKQSGPGL | |
| | VQPSQSLSITCTVSGFSLTNYGVHW | |
| | VRQSPGKGLEWLGVIWSGGNTDYN | |
| | TPFTSRLSINKDNSKSQVFFKMNSLQ | |
| | SQDTAIYYCARALTYYDYEFAYWG | |
| | QGTLVTVSAASTKGPSVFPLAPSSKS | |
| | TSGGTAALGCLVKDYFPEPVTVSW | |
| | NSGALTSGVHTFPAVLQSSGLYSLS | |
| | SVVTVPSSSLGTQTYICNVNHKPSN | |
| | TKVDKKVEPKSC | |
| EGFR TRACTr-1 LC | GGPCRSHIDVAKPICVGGGGSGGLS | 643 |
| | GRSDAGSPLGLAGSGGSDILLTQSP | |
| | VILSVSPGERVSFSCRASQSIGTNIH | |
| | WYQQRINGSPRLLIKYASESISGIPS | |
| | RFSGSGSGTDFTLSINSVESEDIADY | |
| | YCQQNNNWPTTFGAGTKLELKRTV | |
| | AAPSVFIFPPSDEQLKSGTASVVCLL | |
| | NNFYPREAKVQWKVDNALQSGNS | |
| | QESVTEQDSKDSTYSLSSTLTLSKA | |
| | DYEKHKVYACEVTHQGLSSPVTKS | |
| | FNRGEC | |
| EGFR TRACTr-1 HC | EVQLVESGGGLVQPGGSLRLSCAAS | 644 |
| | GSTFYTAVMGWVRQAPGKGLEWV | |
| | AAIRWTALTTSYADSVKGRFTISRD | |
| | GAKTTLYLQMNSLRPEDTAVYYCA | |
| | ARGTLGLFTTADSYDYWGQGTLVT | |
| | VSSGGGGSGGGSGGVYCGPEFDES | |
| | VGCMGGGGSGGGLSGRSDAGSPLG | |
| | LAGSGGGSEVQLVESGGGLVQPGG | |
| | SLKLSCAASGFTFNKYAMNWVRQA | |
| | PGKGLEWVARIRSKYNNYATYYAD | |
| | SVKDRFTISRDDSKNTAYLQMNNL | |
| | KTEDTAVYYCVRHGNFGNSYISYW | |
| | AYWGQGTLVTVSSGGGGSGGGGSG | |
| | GGGSQTVVTQEPSLTVSPGGTVTLT | |
| | CGSSTGAVTSGNYPNWVQQKPGQA | |
| | PRGLIGGTKFLAPGTPARFSGSLLGG | |
| | KAALTLSGVQPEDEAEYYCVLWYS | |
| | NRWVFGGGTKLTVLGGGGSQVQL | |
| | KQSGPGLVQPSQSLSITCTVSGFSLT | |
| | NYGVHWVRQSPGKGLEWLGVIWS | |
| | GGNTDYNTPFTSRLSINKDNSKSQV | |
| | FFKMNSLQSNDTAIYYCARALTYY | |
| | DYEFAYWGQGTLVTVSAASTKGPS | |
| | VFPLAPSSKSTSGGTAALGCLVKDY | |
| | FPEPVTVSWNSGALTSGVHTFPAVL | |
| | QSSGLYSLSSVVTVPSSSLGTQTYIC | |
| | NVNHKPSNTKVDKKVEPKSC | |
| EGFR TRACTr-1 | PCRSHIDVAKPICV | 645 |
| Peptide Mask (P₄) | | |
| EGFR TRACTr-2 | PCLFHFDPAKPICS | 646 |
| Peptide Mask (P₄) | | |

TABLE 16
Tumor antigen binding domain amino acid sequences - anti-PSMA

| | Amino Acid Sequence | SEQ ID |
|---|---|---|
| Construct Description | (N to C) | NO: |
| PSMA: HC: CDR1 | GFAFSRYG | 647 |
| PSMA: HC: CDR2 | IWYDGSNK | 648 |
| PSMA: HC: CDR3 | ARGGDFLYYYYYGMDV | 649 |
| PSMA: LC: CDR1 | QGISNY | 650 |
| PSMA: LC: CDR2 | EA | |
| PSMA: LC: CDR3 | QNYNSAPFT | 652 |
| 006 PSMA Fab LC | DIQMTQSPSSLSASVGDRVTIT | 653 |
| | CRASQGISNYLAWYQQKTGK | |
| | VPKFLIYEASTLQSGVPSRFSG | |
| | GGSGTDFTLTISSLQPEDVATY | |
| | YCQNYNSAPFTFGPGTKVDIK | |
| | RTVAAPSVFIFPPSDEQLKSGT | |
| | ASVVCLLNNFYPREAKVQWK | |
| | VDNALQSGNSQESVTEQDSKD | |
| | STYSLSSTLTLSKADYEKHKV | |
| | YACEVTHQGLSSPVTKSFNRG | |
| | EC | |
| 006 PSMA Fab HC | QVQLVESGGGVVQPGRSLRLS | 654 |
| | CAASGFAFSRYGMHWVRQAP | |
| | GKGLEWVAVIWYDGSNKYY | |
| | ADSVKGRFTISRDNSKNTQYL | |
| | QMNSLRAEDTAVYYCARGGD | |
| | FLYYYYYGMDVWGQGTTVT | |
| | VSSASTKGPSVFPLAPSSKSTS | |
| | GGTAALGCLVKDYFPEPVTVS | |
| | WNSGALTSGVHTFPAVLQSSG | |
| | LYSLSSVVTVPSSSLGTQTYIC | |
| | NVNHKPSNTKVDKKVEPKSC | |
| PSMA TCE-1 LC | EVQLVESGGGLVQPGGSLKLS | 655 |
| | CAASGFTFNKYAMNWVRQAP | |
| | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSDIQMTQSPSSLSAS | |
| | VGDRVTITCRASQGISNYLAW | |
| | YQQKTGKVPKFLIYEASTLQS | |
| | GVPSRFSGGGSGTDFTLTISSL | |
| | QPEDVATYYCQNYNSAPFTFG | |
| | PGTKVDIKRTVAAPSVFIFPPS | |
| | DEQLKSGTASVVCLLNNFYPR | |
| | EAKVQWKVDNALQSGNSQES | |
| | VTEQDSKDSTYSLSSTLTLSKA | |
| | DYEKHKVYACEVTHQGLSSP | |
| | VTKSFNRGEC | |
| PSMA TCE-1 HC | QVQLVESGGGVVQPGRSLRLS | 656 |
| | CAASGFAFSRYGMHWVRQAP | |
| | GKGLEWVAVIWYDGSNKYYA | |
| | DSVKGRFTISRDNSKNTQYLQ | |
| | MNSLRAEDTAVYYCARGGDF | |
| | LYYYYYGMDVWGQGTTVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |
| PSMA TRACTr-1 LC | EVQLVESGGGLVQPGGSLRLS | 657 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSDIQMTQSPSSLSASVGDRVT | |
| | ITCRASQGISNYLAWYQQKTG | |
| | KVPKFLIYEASTLQSGVPSRFS | |
| | GGGSGTDFTLTISSLQPEDVAT | |
| | YYCQNYNSAPFTFGPGTKVDI | |
| | KRTVAAPSVFIFPPSDEQLKSG | |
| | TASVVCLLNNFYPREAKVQW | |
| | KVDNALQSGNSQESVTEQDSK | |
| | DSTYSLSSTLTLSKADYEKHK | |
| | VYACEVTHQGLSSPVTKSFNR | |
| | GEC | |
| PSMA TRACTr-1 HC | QVQLVESGGGVVQPGRSLRLS | 658 |
| | CAASGFAFSRYGMHWVRQAP | |
| | GKGLEWVAVIWYDGSNKYYA | |
| | DSVKGRFTISRDNSKNTQYLQ | |
| | MNSLRAEDTAVYYCARGGDF | |
| | LYYYYYGMDVWGQGTTVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |

TABLE 17
Tumor antigen binding domain amino acid
sequences - anti-TROP2

| | Amino Acid Sequence | SEQ ID |
|---|---|---|
| Construct Description | (N to C) | NO: |
| TROP2: HC: CDR1 | GYTFTNYG | 659 |
| TROP2: HC: CDR2 | INTYTGEP | 660 |
| TROP2: HC: CDR3 | ARGGFGSSYWYFDV | 661 |
| TROP2: HC: CDR3 F108A | ARGGFGSSYWYADV | 662 |
| TROP2: HC: CDR3 D109A | ARGGFGSSYWYFAV | 663 |
| TROP2: LC: CDR1 | QDVSIA | 664 |
| TROP2: LC: CDR2 | SAS | |
| TROP2: LC: CDR3 | QQHYITPLT | 666 |
| TROP2 Fab LC | DIQLTQSPSSLSASVGDRVSITC | 667 |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 Fab HC | QVQLQQSGSELKKPGASVKVS | 668 |
| | CKASGYTFTNYGMNWVKQA | |
| | PGQGLKWMGWINTYTGEPTY | |
| | TDDFKGRFAFSLDTSVSTAYL | |
| | QISSLKADDTAVYFCARGGFG | |
| | SSYWYFDVWGQGSLVTVSSA | |
| | STKGPSVFPLAPSSKSTSGGTA | |
| | ALGCLVKDYFPEPVTVSWNSG | |
| | ALTSGVHTFPAVLQSSGLYSLS | |
| | SVVTVPSSSLGTQTYICNVNH | |
| | KPSNTKVDKKVEPKSC | |
| TROP2 Fab LC | DIQLTQSPSSLSASVGDRVSITC | 669 |
| F108A | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 Fab HC | QVQLQQSGSELKKPGASVKVS | 670 |
| F108A | CKASGYTFTNYGMNWVKQA | |
| | PGQGLKWMGWINTYTGEPTY | |
| | TDDFKGRFAFSLDTSVSTAYL | |
| | QISSLKADDTAVYFCARGGFG | |
| | SSYWYADVWGQGSLVTVSSA | |
| | STKGPSVFPLAPSSKSTSGGTA | |
| | ALGCLVKDYFPEPVTVSWNSG | |
| | ALTSGVHTFPAVLQSSGLYSLS | |
| | SVVTVPSSSLGTQTYICNVNH | |
| | KPSNTKVDKKVEPKSC | |
| TROP2 Fab LC | DIQLTQSPSSLSASVGDRVSITC | 671 |
| D109A | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 Fab HC | QVQLQQSGSELKKPGASVKVS | 672 |
| D109A | CKASGYTFTNYGMNWVKQA | |
| | PGQGLKWMGWINTYTGEPTY | |
| | TDDFKGRFAFSLDTSVSTAYL | |
| | QISSLKADDTAVYFCARGGFG | |
| | SSYWYFAVWGQGSLVTVSSA | |
| | STKGPSVFPLAPSSKSTSGGTA | |
| | ALGCLVKDYFPEPVTVSWNSG | |
| | ALTSGVHTFPAVLQSSGLYSLS | |
| | SVVTVPSSSLGTQTYICNVNH | |
| | KPSNTKVDKKVEPKSC | |
| TROP2 TCE-1 LC | DIQLTQSPSSLSASVGDRVSITC | 673 |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| TROP2 TCE-1 HC | EVQLVESGGGLVQPGGSLKLS | 674 |
| | CAASGFTFNKYAMNWVRQAP | |
| | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSQVQLQQSGSELKKP | |
| | GASVKVSCKASGYTFTNYGM | |
| | NWVKQAPGQGLKWMGWINT | |
| | YTGEPTYTDDFKGRFAFSLDT | |
| | SVSTAYLQISSLKADDTAVYF | |
| | CARGGFGSSYWYFDVWGQGS | |
| | LVTVSSASTKGPSVFPLAPSSK | |
| | STSGGTAALGCLVKDYFPEPV | |
| | TVSWNSGALTSGVHTFPAVLQ | |
| | SSGLYSLSSVVTVPSSSLGTQT | |
| | YICNVNHKPSNTKVDKKVEPK | |
| | SC | |
| TROP2 TCE-2 LC | DIQLTQSPSSLSASVGDRVSITC | 675 |
| F108A | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| TROP2 TCE-2 HC | EVQLVESGGGLVQPGGSLKLS | 676 |
| F108A | CAASGFTFNKYAMNWVRQAP | |
| | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSQVQLQQSGSELKKP | |
| | GASVKVSCKASGYTFTNYGM | |
| | NWVKQAPGQGLKWMGWINT | |
| | YTGEPTYTDDFKGRFAFSLDT | |
| | SVSTAYLQISSLKADDTAVYF | |
| | CARGGFGSSYWYADVWGQGS | |
| | LVTVSSASTKGPSVFPLAPSSK | |
| | STSGGTAALGCLVKDYFPEPV | |
| | TVSWNSGALTSGVHTFPAVLQ | |
| | SSGLYSLSSVVTVPSSSLGTQT | |
| | YICNVNHKPSNTKVDKKVEPK | |
| | SC | |
| TROP2 TCE-3 LC | DIQLTQSPSSLSASVGDRVSITC | 677 |
| D109A | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKRT | |
| | VAAPSVFIFPPSDEQLKSGTAS | |
| | VVCLLNNFYPREAKVQWKVD | |
| | NALQSGNSQESVTEQDSKDST | |
| | YSLSSTLTLSKADYEKHKVYA | |
| | CEVTHQGLSSPVTKSFNRGEC | |
| TROP2 TCE-3 HC | EVQLVESGGGLVQPGGSLKLS | 678 |
| D109A | CAASGFTFNKYAMNWVRQAP | |
| | GKGLEWVARIRSKYNNYATY | |
| | YADSVKDRFTISRDDSKNTAY | |
| | LQMNNLKTEDTAVYYCVRHG | |
| | NFGNSYISYWAYWGQGTLVT | |
| | VSSGGGGSGGGGSGGGGSQT | |
| | VVTQEPSLTVSPGGTVTLTCGS | |
| | STGAVTSGNYPNWVQQKPGQ | |
| | APRGLIGGTKFLAPGTPARFSG | |
| | SLLGGKAALTLSGVQPEDEAE | |
| | YYCVLWYSNRWVFGGGTKLT | |
| | VLGGGGSQVQLQQSGSELKKP | |
| | GASVKVSCKASGYTFTNYGM | |
| | NWVKQAPGQGLKWMGWINT | |
| | YTGEPTYTDDFKGRFAFSLDT | |
| | SVSTAYLQISSLKADDTAVYF | |
| | CARGGFGSSYWYFAVWGQGS | |
| | LVTVSSASTKGPSVFPLAPSSK | |
| | STSGGTAALGCLVKDYFPEPV | |
| | TVSWNSGALTSGVHTFPAVLQ | |
| | SSGLYSLSSVVTVPSSSLGTQT | |
| | YICNVNHKPSNTKVDKKVEPK | |
| | SC | |
| TROP2 TRACTr-1 LC | GGVDFCGLYHWPICYQGGGG | 679 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTr-1 HC | EVQLVESGGGLVQPGGSLRLS | 680 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYFAVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-2 LC | GGVDFCALYHWPICYQGGGG | 681 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTI-2 HC | EVQLVESGGGLVQPGGSLRLS | 682 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYADVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-3 LC | GGVDFCGLYHWPICYQGGGG | 683 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTr-3 HC | EVQLVESGGGLVQPGGSLRLS | 684 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCAGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNAGNSY | |
| | ISYWAYWGQGTLVTVSSGGG | |
| | GSGGGGSGGGGSQTVVTQEPS | |
| | LTVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYFAVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-4 LC | GGVDFCGLYHWPICYQGGGG | 685 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTr-4 HC | EVQLVESGGGLVQPGGSLRLS | 686 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCAGGGGSGGGLSGRSD | |
| | AGSPLGLAGSGGGSEVQLVES | |
| | GGGLVQPGGSLKLSCAASGFT | |
| | FQKYAMNWVRQAPGKGLEW | |
| | VARIRSKYNNYATYYADSVK | |
| | DRFTISRDDSKNTAYLQMNNL | |
| | KTEDTAVYYCVRHGNFGNSY | |
| | VSYWAYWGQGTLVTVSSGGG | |
| | GSGGGGSGGGGSQTVVTQEPS | |
| | LTVSPGGTVTLTCGSSTGAVTS | |
| | ANYPNWVQQKPGQAPRGLIG | |
| | GTKFLAPGTPARFSGSLLGGK | |
| | AALTLSGVQPEDEAEYYCAL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYFAVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-5 LC | GGVDFCALYHWPICYQGGGG | 687 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTr-5 HC | EVQLVESGGGLVQPGGSLRLS | 688 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCAGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNAGNSY | |
| | ISYWAYWGQGTLVTVSSGGG | |
| | GSGGGGSGGGGSQTVVTQEPS | |
| | LTVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYADVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-6 LC | GGVDFCALYHWPICYQGGGG | 689 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTr-6 HC | EVQLVESGGGLVQPGGSLRLS | 690 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCAGGGGSGGGLSGRSD | |
| | AGSPLGLAGSGGGSEVQLVES | |
| | GGGLVQPGGSLKLSCAASGFT | |
| | FQKYAMNWVRQAPGKGLEW | |
| | VARIRSKYNNYATYYADSVK | |
| | DRFTISRDDSKNTAYLQMNNL | |
| | KTEDTAVYYCVRHGNFGNSY | |
| | VSYWAYWGQGTLVTVSSGGG | |
| | GSGGGGSGGGGSQTVVTQEPS | |
| | LTVSPGGTVTLTCGSSTGAVTS | |
| | ANYPNWVQQKPGQAPRGLIG | |
| | GTKFLAPGTPARFSGSLLGGK | |
| | AALTLSGVQPEDEAEYYCAL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYADVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-7 LC | GGVDFCALYHWPICYQGGGG | 691 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTr-7 HC | EVQLVESGGGLVQPGGSLRLS | 692 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCAGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRAGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGGG | |
| | GSQVQLQQSGSELKKPGASVK | |
| | VSCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYADVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-8 LC wt | GGVDFCKIYSWPVCHQGGGG | 4452 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIK | |
| | RTVAAPSVFIFPPSDEQLKSGT | |
| | ASVVCLLNNFYPREAKVQWK | |
| | VDNALQSGNSQESVTEQDSKD | |
| | STYSLSSTLTLSKADYEKHKV | |
| | YACEVTHQGLSSPVTKSFNRG | |
| | EC | |
| TROP2 TRACTr-8 HC wt | EVQLVESGGGLVQPGGSLRLS | 4453 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSY | |
| | ISYWAYWGQGTLVTVSSGGG | |
| | GSGGGGSGGGGSQTVVTQEPS | |
| | LTVSPGGTVTLTCGSSTGAVT | |
| | SGNYPNWVQQKPGQAPRGLI | |
| | GGTKFLAPGTPARFSGSLLGG | |
| | KAALTLSGVQPEDEAEYYCVL | |
| | WYSNRWVFGGGTKLTVLGG | |
| | GGSQVQLQQSGSELKKPGASV | |
| | KVSCKASGYTFTNYGMNWV | |
| | KQAPGQGLKWMGWINTYTG | |
| | EPTYTDDFKGRFAFSLDTSVST | |
| | AYLQISSLKADDTAVYFCARG | |
| | GFGSSYWYFDVWGQGSLVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |
| TROP2 TRACTr-9 LC wt | GGIDFCMLYNWPICAGGGGGS | 4454 |
| | SGGSAAGLLAPPGGLSGRSDA | |
| | GGGGSDIQLTQSPSSLSASVGD | |
| | RVSITCKASQDVSIAVAWYQQ | |
| | KPGKAPKLLIYSASYRYTGVP | |
| | DRFSGSGSGTDFTLTISSLQPE | |
| | DFAVYYCQQHYITPLTFGAG | |
| | TKVEIKRTVAAPSVFIFPPSDE | |
| | QLKSGTASVVCLLNNFYPREA | |
| | KVQWKVDNALQSGNSQESVT | |
| | EQDSKDSTYSLSSTLTLSKADY | |
| | EKHKVYACEVTHQGLSSPVTK | |
| | SFNRGEC | |
| TROP2 TRACTr-9 HC wt | EVQLVESGGGLVQPGGSLRLS | 4455 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGSSGGSAAGLLAPPG | |
| | GLSGRSDAGGGGSEVQLVESG | |
| | GGLVQPGGSLKLSCAASGFTF | |
| | NKYAMNWVRQAPGKGLEWV | |
| | ARIRSKYNNYATYYADSVKD | |
| | RFTISRDDSKNTAYLQMNNLK | |
| | TEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGG | |
| | GGSQVQLQQSGSELKKPGASV | |
| | KVSCKASGYTFTNYGMNWV | |
| | KQAPGQGLKWMGWINTYTG | |
| | EPTYTDDFKGRFAFSLDTSVST | |
| | AYLQISSLKADDTAVYFCARG | |
| | GFGSSYWYFDVWGQGSLVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |
| TROP2 TRACTr-10 LC wt | GGVDFCKIYSWPVCHQGGGG | 4456 |
| | SSGGSAAGLLAPPGGLSGRSD | |
| | AGGGGSDIQLTQSPSSLSASVG | |
| | DRVSITCKASQDVSIAVAWYQ | |
| | QKPGKAPKLLIYSASYRYTGV | |
| | PDRFSGSGSGTDFTLTISSLQPE | |
| | DFAVYYCQQHYITPLTFGAG | |
| | TKVEIKRTVAAPSVFIFPPSDE | |
| | QLKSGTASVVCLLNNFYPREA | |
| | KVQWKVDNALQSGNSQESVT | |
| | EQDSKDSTYSLSSTLTLSKADY | |
| | EKHKVYACEVTHQGLSSPVTK | |
| | SFNRGEC | |
| TROP2 TRACTr-10 HC wt | EVQLVESGGGLVQPGGSLRLS | 4457 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGSSGGSAAGLLAPPG | |
| | GLSGRSDAGGGGSEVQLVESG | |
| | GGLVQPGGSLKLSCAASGFTF | |
| | NKYAMNWVRQAPGKGLEWV | |
| | ARIRSKYNNYATYYADSVKD | |
| | RFTISRDDSKNTAYLQMNNLK | |
| | TEDTAVYYCVRHGNEGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGG | |
| | GGSQVQLQQSGSELKKPGASV | |
| | KVSCKASGYTFTNYGMNWV | |
| | KQAPGQGLKWMGWINTYTG | |
| | EPTYTDDFKGRFAFSLDTSVST | |
| | AYLQISSLKADDTAVYFCARG | |
| | GFGSSYWYFDVWGQGSLVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |
| TROP2 TRACTr-11 LC wt | EVQLVESGGGLVQPGGSLRLS | 4458 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGSSGGSAAGLLAPPG | |
| | GLSGRSDAGGGGSEVQLVESG | |
| | GGLVQPGGSLKLSCAASGFTF | |
| | NKYAMNWVRQAPGKGLEWV | |
| | ARIRSKYNNYATYYADSVKD | |
| | RFTISRDDSKNTAYLQMNNLK | |
| | TEDTAVYYCVRHGNFGNSYI | |
| | SYWAYWGQGTLVTVSSGGGG | |
| | SGGGGSGGGGSQTVVTQEPSL | |
| | TVSPGGTVTLTCGSSTGAVTS | |
| | GNYPNWVQQKPGQAPRGLIG | |
| | GTKFLAPGTPARFSGSLLGGK | |
| | AALTLSGVQPEDEAEYYCVL | |
| | WYSNRWVFGGGTKLTVLGG | |
| | GGSDIQLTQSPSSLSASVGDRV | |
| | SITCKASQDVSIAVAWYQQKP | |
| | GKAPKLLIYSASYRYTGVPDR | |
| | FSGSGSGTDFTLTISSLQPEDFA | |
| | VYYCQQHYITPLTFGAGTKV | |
| | EIKRTVAAPSVFIFPPSDEQLKS | |
| | GTASVVCLLNNFYPREAKVQ | |
| | WKVDNALQSGNSQESVTEQD | |
| | SKDSTYSLSSTLTLSKADYEKH | |
| | KVYACEVTHQGLSSPVTKSFN | |
| | RGEC | |
| TROP2 TRACTr-11 HC wt | GGIDFCMLYNWPICAGGGGGS | 4459 |
| | SGGSAAGLLAPPGGLSGRSDA | |
| | GGGGSQVQLQQSGSELKKPG | |
| | ASVKVSCKASGYTFTNYGMN | |
| | WVKQAPGQGLKWMGWINTY | |
| | TGEPTYTDDFKGRFAFSLDTS | |
| | VSTAYLQISSLKADDTAVYFC | |
| | ARGGFGSSYWYFDVWGQGS | |
| | LVTVSSASTKGPSVFPLAPSSK | |
| | STSGGTAALGCLVKDYFPEPV | |
| | TVSWNSGALTSGVHTFPAVLQ | |
| | SSGLYSLSSVVTVPSSSLGTQT | |
| | YICNVNHKPSNTKVDKKVEPK | |
| | SC | |
| TROP2 TRACTr-12 LC wt | GGIDFCMLYNWPICAGGGGGS | 4460 |
| | GGLSGRSDAGSPLGLAGSGGS | |
| | DIQLTQSPSSLSASVGDRVSITC | |
| | KASQDVSIAVAWYQQKPGKA | |
| | PKLLIYSASYRYTGVPDRFSGS | |
| | GSGTDFTLTISSLQPEDFAVYY | |
| | CQQHYITPLTFGAGTKVEIKR | |
| | TVAAPSVFIFPPSDEQLKSGTA | |
| | SVVCLLNNFYPREAKVQWKV | |
| | DNALQSGNSQESVTEQDSKDS | |
| | TYSLSSTLTLSKADYEKHKVY | |
| | ACEVTHQGLSSPVTKSFNRGE | |
| | C | |
| TROP2 TRACTr-12 HC wt | EVQLVESGGGLVQPGGSLRLS | 4461 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSY | |
| | ISYWAYWGQGTLVTVSSGGG | |
| | GSGGGGSGGGGSQTVVTQEPS | |
| | LTVSPGGTVTLTCGSSTGAVT | |
| | SGNYPNWVQQKPGQAPRGLI | |
| | GGTKFLAPGTPARFSGSLLGG | |
| | KAALTLSGVQPEDEAEYYCVL | |
| | WYSNRWVFGGGTKLTVLGG | |
| | GGSQVQLQQSGSELKKPGASV | |
| | KVSCKASGYTFTNYGMNWV | |
| | KQAPGQGLKWMGWINTYTG | |
| | EPTYTDDFKGRFAFSLDTSVST | |
| | AYLQISSLKADDTAVYFCARG | |
| | GFGSSYWYFDVWGQGSLVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |
| TROP2 TRACTr-13 LC wt | GGVDFCGLYHWPICYQGGGG | 4462 |
| | SGGLSGRSDAGSPLGLAGSGG | |
| | SDIQLTQSPSSLSASVGDRVSIT | |
| | CKASQDVSIAVAWYQQKPGK | |
| | APKLLIYSASYRYTGVPDRFSG | |
| | SGSGTDFTLTISSLQPEDFAVY | |
| | YCQQHYITPLTFGAGTKVEIK | |
| | RTVAAPSVFIFPPSDEQLKSGT | |
| | ASVVCLLNNFYPREAKVQWK | |
| | VDNALQSGNSQESVTEQDSKD | |
| | STYSLSSTLTLSKADYEKHKV | |
| | YACEVTHQGLSSPVTKSFNRG | |
| | EC | |
| TROP2 TRACTr-13 HC wt | EVQLVESGGGLVQPGGSLRLS | 4463 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSY | |
| | ISYWAYWGQGTLVTVSSGGG | |
| | GSGGGGSGGGGSQTVVTQEPS | |
| | LTVSPGGTVTLTCGSSTGAVT | |
| | SGNYPNWVQQKPGQAPRGLI | |
| | GGTKFLAPGTPARFSGSLLGG | |
| | KAALTLSGVQPEDEAEYYCVL | |
| | WYSNRWVFGGGTKLTVLGG | |
| | GGSQVQLQQSGSELKKPGASV | |
| | KVSCKASGYTFTNYGMNWV | |
| | KQAPGQGLKWMGWINTYTG | |
| | EPTYTDDFKGRFAFSLDTSVST | |
| | AYLQISSLKADDTAVYFCARG | |
| | GFGSSYWYFDVWGQGSLVTV | |
| | SSASTKGPSVFPLAPSSKSTSG | |
| | GTAALGCLVKDYFPEPVTVSW | |
| | NSGALTSGVHTFPAVLQSSGL | |
| | YSLSSVVTVPSSSLGTQTYICN | |
| | VNHKPSNTKVDKKVEPKSC | |
| TROP2 TRACTr-14 LC wt | EVQLVESGGGLVQPGGSLRLS | 4464 |
| | CAASGSTFYTAVMGWVRQAP | |
| | GKGLEWVAAIRWTALTTSYA | |
| | DSVKGRFTISRDGAKTTLYLQ | |
| | MNSLRPEDTAVYYCAARGTL | |
| | GLFTTADSYDYWGQGTLVTV | |
| | SSGGGGSGGGSGGVYCGPEFD | |
| | ESVGCMGGGGSGGGLSGRSD | |
| | AGSPLGLAGSGGGSEVQLVES | |
| | GGGLVQPGGSLKLSCAASGFT | |
| | FNKYAMNWVRQAPGKGLEW | |
| | VARIRSKYNNYATYYADSVK | |
| | DRFTISRDDSKNTAYLQMNNL | |
| | KTEDTAVYYCVRHGNFGNSY | |
| | ISYWAYWGQGTLVTVSSGGG | |
| | GSGGGGSGGGGSQTVVTQEPS | |
| | LTVSPGGTVTLTCGSSTGAVT | |
| | SGNYPNWVQQKPGQAPRGLI | |
| | GGTKFLAPGTPARFSGSLLGG | |
| | KAALTLSGVQPEDEAEYYCVL | |
| | WYSNRWVFGGGTKLTVLGG | |
| | GGSDIQLTQSPSSLSASVGDRV | |
| | SITCKASQDVSIAVAWYQQKP | |
| | GKAPKLLIYSASYRYTGVPDR | |
| | FSGSGSGTDFTLTISSLQPEDFA | |
| | VYYCQQHYITPLTFGAGTKV | |
| | EIKRTVAAPSVFIFPPSDEQLKS | |
| | GTASVVCLLNNFYPREAKVQ | |
| | WKVDNALQSGNSQESVTEQD | |
| | SKDSTYSLSSTLTLSKADYEKH | |
| | KVYACEVTHQGLSSPVTKSFN | |
| | RGEC | |
| TROP2 TRACTr-14 HC wt | GGVDFCGLYHWPICYQGGGG | 4465 |
| | SGGLSGRSDAGSPLGLAGSGG | |
| | SQVQLQQSGSELKKPGASVKV | |
| | SCKASGYTFTNYGMNWVKQ | |
| | APGQGLKWMGWINTYTGEPT | |
| | YTDDFKGRFAFSLDTSVSTAY | |
| | LQISSLKADDTAVYFCARGGF | |
| | GSSYWYFDVWGQGSLVTVSS | |
| | ASTKGPSVFPLAPSSKSTSGGT | |
| | AALGCLVKDYFPEPVTVSWNS | |
| | GALTSGVHTFPAVLQSSGLYS | |
| | LSSVVTVPSSSLGTQTYICNVN | |
| | HKPSNTKVDKKVEPKSC | |
| TROP2 TRACTr-1 Peptide Mask | IDFCMLYNWPICA | 693 |
| (P₄) | | |
| TROP2 TRACTr-2 Peptide Mask | VDFCGLYHWPICYQ | 694 |
| (P₄) | | |
| TROP2 TRACTr-2 Peptide Mask | VDFCALYHWPICYQ | 695 |
| (P₄) | | |

In some embodiments, the T cell engager molecule is selectively activated in tumor microenvironments. In some embodiments, the T cell engager is according to the following subformula: P₃-L₃-D-L₀-E (Formula IIa) wherein D comprises a CD3 binding domain; E comprises the tumor antigen binding domain; L₀ comprises the linker that connects D to E; P₃ comprises a peptide that binds to D and L₃ comprises a linking moiety that connects D to P₃ and is a substrate for a tumor specific protease. In some embodiments, the T cell engager is according to the following subformula: D-L₀-E-L₄-P₄ (Formula IIb) wherein D comprises a CD3 binding domain; E comprises the tumor antigen binding domain; L₀ comprises the linker that connects D to E; P₄ comprises a peptide that binds to E and L₄ comprises a linking moiety that connects E to P₄ and is a substrate for a tumor specific protease. In some embodiments, the T cell engager is according to the following subformula: P₃-L₃-D-L₀-E-L₄-P₄ (Formula IIc) wherein D comprises a CD3 binding domain; E comprises the tumor antigen binding domain; L₀ comprises the linker that connects D to E; P₃ comprises a peptide that binds to D and L₃ comprises a linking moiety that connects D to P₃ and is a substrate for a tumor specific protease; P₄ comprises a peptide that binds to E and L₄ comprises a linking moiety that connects E to P₄ and is a substrate for a tumor specific protease.

In some embodiments, the T cell engager comprises Hi. In some embodiments, H₁ comprises a sequence according to SEQ ID NO: 141 or SEQ ID NO: 146. In some embodiments, H₁ comprises a single domain antibody. In some embodiments, the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140.

In some embodiments, L₃ or L₄ is a peptide sequence having at least 5 to no more than 50 amino acids. In some embodiments, L₃ or Ia is a peptide sequence having at least 10 to no more than 30 amino acids. In some embodiments, L₃ or Ia is a peptide sequence having at least 10 amino acids. In some embodiments, L₃ or L₄ is a peptide sequence having at least 18 amino acids. In some embodiments, L₃ or L₄ is a peptide sequence having at least 26 amino acids. In some embodiments, L₃ or La has a formula comprising (G₂S)ₙ, wherein n is an integer from 1 to 3 (SEQ ID NO: 605). In some embodiments, L₃ or L₄ has a formula comprising (G₂S)ₙ. wherein n is an integer of at least 1. In some embodiments, L₃ or L₄has a formula selected from the group consisting of (G₂S)ₙ, (GS)ₙ, (GSGGS). (SEQ ID NO: 606), (GGGS), (SEQ ID NO: 607).(GGGGS), (SEQ ID NO: 608), and (GSSGGS), (SEQ ID NO: 609). wherein n is an integer of at least 1.

In some embodiments, the tumor specific protease is selected from the group consisting of metalloprotease, serine protease, cysteine protease, threonine protease, and aspartic protease. In some embodiments, L₃ or L₄ comprises a urokinase cleavable amino acid sequence, a matriptase cleavable amino acid sequence, or a matrix metalloprotease cleavable amino acid sequence. In some embodiments, L₃ or L₄ comprises a sequence according to any one of SEQ ID NOs: 21-22 and 107-136. In some embodiments. L₃ is bound to N-terminus of D. In some embodiments, L₃ is bound to C-terminus of D. In some embodiments, L₄ is bound to N-terminus of E. In some embodiments, L₄ is bound to C-terminus of E.

In some embodiments, P₃ becomes unbound from D when L₃ is cleaved by the tumor specific protease thereby exposing D to CD3. In some embodiments, P₄ becomes unbound from E when L₄ is cleaved by the tumor specific protease thereby exposing E to the tumor antigen. In some embodiments, P; impairs binding of D to CD3. In some embodiments, P₃ is bound to D through ionic interactions, electrostatic interactions, hydrophobic interactions, Pi-stacking interactions, and H-bonding interactions, or a combination thereof. In some embodiments, P₃ is bound to D at or near an antigen binding site. In some embodiments, P₃ becomes unbound from D when L₃ is cleaved by the tumor specific protease thereby exposing D to CD3.

In some embodiments, P₃ has less than 70% sequence identity to CD3. In some embodiments, P₃ has less than 70% sequence identity to CD3. In some embodiments, P₃ has less than 85% sequence identity to CD3. In some embodiments, P₃ has less than 90% sequence identity to CD3. In some embodiments, P₃ has less than 95% sequence identity to CD3. In some embodiments. P₃ has less than 98% sequence identity to CD3. In some embodiments, P₃ has less than 99% sequence identity to CD3. In some embodiments, P₃ comprises the amino acid sequence according to SEQ ID NO: 625 or SEQ ID NO: 626. In some embodiments, P₃ comprises a de novo amino acid sequence that shares less than 10% sequence identity to CD3.

In some embodiments, P₄ impairs binding of E to the tumor antigen. In some embodiments, P₄ is bound to E through ionic interactions, electrostatic interactions, hydrophobic interactions, Pi-stacking interactions, and H-bonding interactions, or a combination thereof. In some embodiments, P₄ is bound to E at or near an antigen binding site. In some embodiments, P₄ becomes unbound from E when L₄ is cleaved by the tumor specific protease thereby exposing E to the tumor antigen. In some embodiments, P₄ has less than 70% sequence identity to the tumor antigen. In some embodiments, P₄ has less than 80% sequence identity to the tumor antigen. In some embodiments, P₄ has less than 85% sequence identity to the tumor antigen. In some embodiments, P₄ has less than 90% sequence identity to the tumor antigen. In some embodiments, P₄ has less than 95% sequence identity to the tumor antigen. In some embodiments, P₄ comprises a de novo amino acid sequence that shares less than 10% sequence identity to the tumor antigen.

In some embodiments, P₃ or P₄ comprises a peptide sequence of at least 5 amino acids in length. In some embodiments, P₃ or P₄ comprises a peptide sequence of at least 6 amino acids in length. In some embodiments, P₃ or P₄ comprises a peptide sequence of at least 10 amino acids in length. In some embodiments, P₃ or P₄ comprises a peptide sequence of at least 10 amino acids in length and no more than 20 amino acids in length. In some embodiments, P₃ or P₄ comprises a peptide sequence of at least 16 amino acids in length. In some embodiments, P₃ or P₄ comprises a peptide sequence of no more than 40 amino acids in length. In some embodiments, P₃ or P₄ comprises at least two cysteine amino acid residues. In some embodiments, P₃ or P₄ comprises a cyclic peptide or a linear peptide. In some embodiments, P₃ or P₄ comprises a cyclic peptide. In some embodiments, P₃ or P₄ comprises a linear peptide.

In some embodiments, P₄ comprises the amino acid sequence according to SEQ ID NO: 645. In some embodiments, the tumor antigen comprises EGFR, and the T cell engager comprises the amino acid sequences of SEQ ID NOs: 643 and 644.

In some embodiments, P₄ comprises the amino acid sequence according to any one of SEQ ID NOs: 693-695. In some embodiments, the tumor antigen comprises TROP2, and the T cell engager comprises the amino acid sequences of SEQ ID NOs: 679 and 680, SEQ ID NOs: 681 and 682, SEQ ID NOs: 683 and 684, SEQ ID NOs: 685 and 686, SEQ ID NOs: 687 and 688, SEQ ID NOs: 689 and 690, and SEQ ID NOs: 691 and 692. In some embodiments, the tumor antigen comprises TROP2, and the T cell engager comprises the amino acid sequences of SEQ ID NOs: 4452 and 4453, SEQ ID NOs: 4454 and 4455, SEQ ID NOs: 4456 and 4457, SEQ ID NOs: 4458 and 4459, SEQ ID NOs: 4460 and 4461, SEQ ID NOs: 4462 and 4463, or SEQ ID NOs: 4464 and 4465.

In some embodiments, the tumor antigen comprises PSMA, and the T cell engager comprises the amino acid sequences of SEQ ID NOs: 657 and 658.

### Production of Antibodies

In some embodiments, the antibodies or antigen binding fragments thereof as described herein are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding fragment is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988. Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody. or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For longterm, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. NatI. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson. 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression. A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1). In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

Disclosed herein, in some embodiments, are vectors comprising an isolated recombinant nucleic acid molecule encoding an isolated polypeptide or polypeptide complex of any one of the embodiments disclosed herein.

Disclosed herein, in some embodiments, are vectors comprising an isolated recombinant nucleic acid molecule encoding a polypeptide or polypeptide complex that comprises a first antigen recognizing molecule that binds to CD28 and a second antigen recognizing molecule that binds to prostate-specific membrane antigen. In some embodiments, the first antigen recognizing molecule that binds to CD28 comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6. In some embodiments, the anti-CD28 heavy chain variable domain comprises an amino acid sequence with at least 80%, 85%, 90%, 95%, 98/, 99%, or 100% sequence identity to SEQ ID NO: 7, and wherein the anti-CD28 light chain variable domain comprises an amino acid sequence with at least 80%, 85%, 90%, 95%, 98%, 99%, or 100% sequence identity to SEQ ID NO: 8. In some embodiments, the anti-CD28 antibody comprises a single chain variable fragment (scFv), a single domain antibody, a Fab, or a Fab′. In some embodiments, the anti-CD28 antibody comprises the scFv. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 9. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 10. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 11. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 12.

In some embodiments, the second antigen recognizing molecule that binds to PSMA comprises an anti-PSMA antibody. In some embodiments, the anti-PSMA antibody comprises an anti-PSMA heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 13, HC-CDR2: SEQ ID NO: 14, and HC-CDR3: SEQ ID NO: 15 and the anti-PSMA antibody comprises an anti-PSMA light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprise an amino acid sequence of LC-CDR1: SEQ ID NO: 16, LC-CDR2: (EA), and LC-CDR3: SEQ ID NO: 18. In some embodiments, the anti-PSMA heavy chain variable domain comprises an amino acid sequence with at least 80%, 85%, 90%, 95%, 980, 99%, or 100% sequence identity to SEQ ID NO: 20, and the anti-PSMA light chain variable domain comprises an amino acid sequence with at least 80%, 85%, 90%, 95%, 98%, 99%, or 100% sequence identity to SEQ ID NO: 19. In some embodiments, the anti-PSMA antibody comprises a single chain variable fragment (scFv), a single domain antibody, a Fab, or a Fab′. In some embodiments, the anti-PSMA antibody comprises the Fab or Fab′. In some embodiments, the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 19 and an amino acid sequence with at least at least 80%, 85%, 90%, 95%, 98%, 99%, or 100% sequence identity to SEQ ID NO: 20.

In some embodiments, vectors include any suitable vectors derived from either eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pE™ vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBacl, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 Ml1. pVL1393 M12, FLAG vectors such as pPolh-FLAG1 or pPolh-MAT 2, or MAT vectors such as pPolh-MAT1, or pPolh-MAT2.

In some cases, yeast vectors include Gatewav® pDEST™ 14 vector, Gateway®, pDEST™ 15 vector, Gateway® pDEST™ 17 vector, Gatewayt pDEST™ 24 vector, Gateway® pYES-DEST52 vector, pBAD-DEST49 Gateway® destination vector, pA0815 Pichia vector, pFLDI Pichi pastoris vector, pGAPZA,B, & C Pichia pastoris vector, pPIC3.5K Pichia vector, pPIC6 A, B, & C Pichia vector, pPIC9K Pichia vector, pTEFI/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress® R.

### Host Cells

Disclosed herein, in some embodiments, are host cells comprising an isolated polypeptide or polypeptide complex of any one of the embodiments disclosed herein.

Disclosed herein, in some embodiments, are host cells comprising an isolated polypeptide or polypeptide complex that comprises a first antigen recognizing molecule that binds to CD28 and a second antigen recognizing molecule that binds to prostate-specific membrane antigen. In some embodiments, the first antigen recognizing molecule that binds to CD28 comprises an anti-CD28 antibody. In some embodiments, the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6. In some embodiments, the anti-CD28 heavy chain variable domain comprises an amino acid sequence with at least 80%, 85%, 90%, 95%, 98%, 99%, or 100% sequence identity to SEQ ID NO: 7, and wherein the anti-CD28 light chain variable domain comprises an amino acid sequence with at least 80%, 85%, 90%, 95%, 98%, 99%, or 100% sequence identity to SEQ ID NO: 8. In some embodiments, the anti-CD28 antibody comprises a single chain variable fragment (scFv), a single domain antibody, a Fab, or a Fab′. In some embodiments, the anti-CD28 antibody comprises the scFv. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 9. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 10. In some embodiments, the anti-CD28 antibody comprises the scFv, and the scFv comprises an amino acid sequence with at least 80%, 85%, 90%, 95%, 98%, 99%, or 100% sequence identity to SEQ ID NO: 11. In some embodiments, the anti-CD28 antibody comprises the scFv, and the seFv comprises an amino acid sequence with at least 80%, 85%, 90%, 95%, 98%, 99%, or 100% sequence identity to SEQ ID NO: 12.

In some embodiments, the second antigen recognizing molecule that binds to PSMA comprises an anti-PSMA antibody. In some embodiments, the anti-PSMA antibody comprises an anti-PSMA heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 13, HC-CDR2: SEQ ID NO: 14, and HC-CDR3: SEQ ID NO: 15 and the anti-PSMA antibody comprises an anti-PSMA light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprise an amino acid sequence of LC-CDR1: SEQ ID NO: 16, LC-CDR2: (EA), and LC-CDR3: SEQ ID NO: 18. In some embodiments, the anti-PSMA heavy chain variable domain comprises an amino acid sequence with at least 80%, 85%, 90%, 95%, 98%, 99%, or 100% sequence identity to SEQ ID NO: 20, and the anti-PSMA light chain variable domain comprises an amino acid sequence with at least 80%, 85%, 90%, 95%, 98%, 99%, or 100% sequence identity to SEQ ID NO: 19. In some embodiments, the anti-PSMA antibody comprises a single chain variable fragment (scFv), a single domain antibody, a Fab, or a Fab′. In some embodiments, the anti-PSMA antibody comprises the Fab or Fab′. In some embodiments, the anti-PSMA antibody comprises the Fab or Fab′, and the Fab or Fab′ comprises an amino acid sequence with at least 80%, 85%, 90/, 95%, 98%, 99%, or 100% sequence identity to SEQ ID NO: 19 and an amino acid sequence with at least at least 80%, 85%, 90%, 95%, 98%, 99%, or 100% sequence identity to SEQ ID NO: 20.

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™, TOP10. DH5α, DHIOBac™, OmniMax™, MegaX™, DHI2ST™, INV110, TOPIOF™, INVaF, TOPIO/P₃, ccdB Survival, PIR1, PIR2, Stbl2T %. Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast. Fungi include ascomycetes such as yeast, mold, filamentous fungi, basidiomvcetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesci, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVScl.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™-REx™ 293 cell line. Flp-In™-293 cell line. Flp-In™-L₃T3 cell line, Flp-In™-BHK cell line, Flp-In™-CHO cell line, Flp-In™-CV-1 cell line, Flp-In™-J11rkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™ ?1-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division. Exemplary insect host cells include, but are not limited to, Drosophila S2 cells, Sf9 cells, Sf21 cells, High Five™ cells, and expresSF+® cells. In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an isolate polypeptide or polypeptide complex of any embodiment disclosed herein. The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Embodiments

Embodiment 1 comprises an isolated polypeptide or polypeptide complex that comprises a first antigen recognizing molecule that binds to CD28 and a second antigen recognizing molecule that binds to prostate-specific membrane antigen (PSMA).

Embodiment 2 comprises the isolated polypeptide or polypeptide complex of embodiment 1, wherein the first antigen recognizing molecule that binds to CD28 comprises an anti-CD28 antibody.

Embodiment 3 comprises the isolated polypeptide or polypeptide complex of embodiment 2, wherein the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6.

Embodiment 4 comprises the isolated polypeptide or polypeptide complex of embodiment 3, wherein the anti-CD28 heavy chain variable domain comprises an amino acid sequence with at least 80%, 85%, 90%, 95%, 98%, 99%, or 100% sequence identity to SEQ ID NO: 7, and wherein the anti-CD28 light chain variable domain comprises an amino acid sequence with at least 80%, 85%, 90%, 95%, 98%, 99%, or 100% sequence identity to SEQ ID NO: 8.

Embodiment 5 comprises the isolated polypeptide or polypeptide complex of any one of embodiments 2-4, wherein the anti-CD28 antibody comprises a single chain variable fragment (scFv), a single domain antibody, a Fab, a Fab, or a CrossFab.

Embodiment 6 comprises the isolated polypeptide or polypeptide complex of embodiment 5, wherein the anti-CD28 antibody comprises the scFv.

Embodiment 7 comprises the isolated polypeptide of embodiment 5, wherein the anti-CD28 antibody comprises a single chain variable fragment (scFv) with an engineered disulfide.

Embodiment 8 comprises the isolated polypeptide or polypeptide complex of embodiment 5, wherein the anti-CD28 antibody comprises the scFv, and the scFv comprises an amino acid sequence with at least 80%, 85%, 90%, 95%, 98%, 99%, or 100% sequence identity to SEQ ID NO: 9.

Embodiment 9 comprises the isolated polypeptide or polypeptide complex of embodiment 5, wherein the anti-CD28 antibody comprises the scFv, and the scFv comprises an amino acid sequence with at least 80%, 85%, 90%, 95%, 98%, 99%, or 100% sequence identity to SEQ ID NO: 10.

Embodiment 10 comprises the isolated polypeptide or polypeptide complex of embodiment 5, wherein the anti-CD28 antibody comprises the scFv, and the scFv comprises an amino acid sequence with at least 80%, 85%, 90%, 95%, 98%, 99%, or 100% sequence identity to SEQ ID NO: 11.

Embodiment 11 comprises the isolated polypeptide or polypeptide complex of embodiment 5, wherein the anti-CD28 antibody comprises the scFv, and the scFv comprises an amino acid sequence with at least 80%, 85%, 90%, 95%, 98%, 99%, or 100% sequence identity to SEQ ID NO: 12.

Embodiment 12 comprises the isolated polypeptide or polypeptide complex of embodiment 5, wherein the anti-CD28 antibody comprises the Fab.

Embodiment 13 comprises the isolated polypeptide or polypeptide complex of embodiment 12, wherein the Fab comprises an amino acid sequence with at least 80%, 85%, 90%, 95%, 98%, 99%, or 100% sequence identity to SEQ ID NO: 696 and an amino acid sequence with at least 80%, 85%, 90%, 95%, 98%, 99%, or 100% sequence identity to SEQ ID NO: 697.

Embodiment 14 comprises the isolated polypeptide or polypeptide complex of embodiment 5, wherein the anti-CD28 antibody comprises the CrossFab.

Embodiment 15 comprises the isolated polypeptide or polypeptide complex of embodiment 14, wherein the CrossFab comprises an amino acid sequence with at least 80%, 85%, 90%, 95%, 98%, 99%, or 100% sequence identity to SEQ ID NO: 698 and an amino acid sequence with at least 80%, 85%, 90%, 95%, 98%, 99%, or 100% sequence identity to SEQ ID NO: 699.

Embodiment 16 comprises the isolated polypeptide or polypeptide complex of embodiment 14. wherein the CrossFab comprises an amino acid sequence with at least 80%, 85%, 90%, 95%, 98%, 99%, or 100% sequence identity to SEQ ID NO: 700 and an amino acid sequence with at least 80%, 85%, 90%, 95%, 98%, 99%, or 100% sequence identity to SEQ ID NO: 701.

Embodiment 17 comprises the isolated polypeptide or polypeptide complex of any one of the preceding embodiments, wherein the second antigen recognizing molecule that binds to PSMA comprises an anti-PSMA antibody.

Embodiment 18 comprises the isolated polypeptide or polypeptide complex of embodiment 17 wherein the anti-PSMA antibody comprises an anti-PSMA heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 13, HC-CDR2: SEQ ID NO: 14, and HC-CDR3: SEQ ID NO: 15 and the anti-PSMA antibody comprises an anti-PSMA light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprise an amino acid sequence of LC-CDR1: SEQ ID NO: 16, LC-CDR2: (EA), and LC-CDR3: SEQ ID NO: 18.

Embodiment 19 comprises the isolated polypeptide or polypeptide complex of embodiment 18, wherein the anti-PSMA heavy chain variable domain comprises an amino acid sequence with at least 80%, 85%, 90%, 95%, 98%, 99%, or 100% sequence identity to SEQ ID NO: 20, and wherein the anti-PSMA light chain variable domain comprises an amino acid sequence with at least 80%, 85%, 90%, 95%, 98%, 99%, or 100% sequence identity to SEQ ID NO: 19.

Embodiment 20 comprises the isolated polypeptide or polypeptide complex of any one of embodiments 17-19, wherein the anti-PSMA antibody comprises a single chain variable fragment (scFv), a single domain antibody, a Fab, a Fab, or a CrossFab.

Embodiment 21 comprises the isolated polypeptide or polypeptide complex of embodiment 20, wherein the anti-PSMA antibody comprises the Fab or Fab′.

Embodiment 22 comprises the isolated polypeptide or polypeptide complex of embodiment 20, wherein the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 19 and an amino acid sequence with at least at least 80%, 85%, 90%, 95%, 98%, 99%, or 100% sequence identity to SEQ ID NO: 20.

Embodiment 23 comprises the isolated polypeptide or polypeptide complex of embodiment 20, wherein the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 702 and an amino acid sequence with at least at least 80%, 85%, 90%, 95%, 98%, 99%, or 100% sequence identity to SEQ ID NO: 703.

Embodiment 24 comprises the isolated polypeptide or polypeptide complex of embodiment 20, wherein the anti-PSMA antibody comprises the Fab or Fab, and the Fab or Fab′ comprises an amino acid sequence with at least 80%, 85%, 90%, 95%, 98%, 99%, or 100% sequence identity to SEQ ID NO: 704 and an amino acid sequence with at least at least 80%, 85%, 90%, 95%, 98%, 99%, or 100% sequence identity to SEQ ID NO: 705.

Embodiment 25 comprises the isolated polypeptide or polypeptide complex of any one of embodiments 17-24, wherein the anti-CD28 antibody comprises a scFv and the anti-PSMA antibody comprises a Fab or Fab′.

Embodiment 26 comprises the isolated polypeptide or polypeptide complex of embodiment 25, wherein the scFv and the Fab or Fab′ are connected through a linker and the Fab or Fab′ comprise a Fab heavy chain polypeptide and a Fab light chain polypeptide.

Embodiment 27 comprises the isolated polypeptide or polypeptide complex of embodiment 26, wherein the linker connects the scFv to the Fab heavy chain polypeptide.

Embodiment 28 comprises the isolated polypeptide or polypeptide complex of embodiment 26, wherein the linker connects the scFv to the Fab light chain polypeptide.

Embodiment 29 comprises the isolated polypeptide or polypeptide complex of embodiment 26, wherein the linker connects the scFv to the N-term of the Fab heavy chain polypeptide.

Embodiment 30 comprises the isolated polypeptide or polypeptide complex of embodiment 26, wherein the linker connects the scFv to the C-term of the Fab heavy chain polypeptide.

Embodiment 31 comprises the isolated polypeptide or polypeptide complex of embodiment 26, wherein the linker connects the scFv to the N-term of the Fab light chain polypeptide.

Embodiment 32 comprises the isolated polypeptide or polypeptide complex of embodiment 26, wherein the linker connects the scFv to the C-term of the Fab light chain polypeptide.

Embodiment 33 comprises the isolated polypeptide or polypeptide complex of embodiment 26, wherein the linker connects the anti-CD28 heavy chain variable domain of the scFv to the Fab heavy chain polypeptide.

Embodiment 34 comprises the isolated polypeptide or polypeptide complex of embodiment 26, wherein the linker connects the anti-CD28 heavy chain variable domain of the scFv to the Fab light chain polypeptide.

Embodiment 35 comprises the isolated polypeptide or polypeptide complex of embodiment 26, wherein the linker connects the anti-CD28 heavy chain variable domain of the scFv to the N-term of the Fab heavy chain polypeptide.

Embodiment 36 comprises the isolated polypeptide or polypeptide complex of embodiment 26, wherein the linker connects the anti-CD28 heavy chain variable domain of the scFv to the C-term of the Fab heavy chain polypeptide.

Embodiment 37 comprises the isolated polypeptide or polypeptide complex of embodiment 26, wherein the linker connects the anti-CD28 heavy chain variable domain of the scFv to the N-term of the Fab light chain polypeptide.

Embodiment 38 comprises the isolated polypeptide or polypeptide complex of embodiment 26, wherein the linker connects the anti-CD28 heavy chain variable domain of the scFv to the C-term of the Fab light chain polypeptide.

Embodiment 39 comprises the isolated polypeptide or polypeptide complex of embodiment 26, wherein the linker connects the anti-CD28 light chain variable domain of the scFv to the Fab heavy chain polypeptide.

Embodiment 40 comprises the isolated polypeptide or polypeptide complex of embodiment 26, wherein the linker connects the anti-CD28 light chain variable domain of the scFv to the Fab light chain polypeptide.

Embodiment 41 comprises the isolated polypeptide or polypeptide complex of embodiment 26, wherein the linker connects the anti-CD28 light chain variable domain of the scFv to the N-term of the Fab heavy chain polypeptide.

Embodiment 42 comprises the isolated polypeptide or polypeptide complex of embodiment 26, wherein the linker connects the anti-CD28 light chain variable domain of the scFv to the C-term of the Fab heavy chain polypeptide.

Embodiment 43 comprises the isolated polypeptide or polypeptide complex of embodiment 26, wherein the linker connects the anti-CD28 light chain variable domain of the scFv to the N-term of the Fab light chain polypeptide.

Embodiment 44 comprises the isolated polypeptide or polypeptide complex of embodiment 26, wherein the linker connects the anti-CD28 light chain variable domain of the scFv to the C-term of the Fab light chain polypeptide.

Embodiment 45 comprises the isolated polypeptide or polypeptide complex of any one of embodiments 17-24, wherein the anti-CD28 antibody comprises a Fab or Fab′ and the anti-PSMA antibody comprises a Fab or Fab′.

Embodiment 46 comprises the isolated polypeptide or polypeptide complex of embodiment 45, wherein the Fab or Fab′ of the anti-CD28 antibody and the Fab or Fab′ of the anti-PSMA antibody are connected through a linker and the Fab or Fab′ of the anti-PSMA antibody comprise a Fab heavy chain polypeptide and a Fab light chain polypeptide.

Embodiment 47 comprises the isolated polypeptide or polypeptide complex of embodiment 46, wherein the linker connects the Fab or Fab′ of the anti-CD28 antibody to the Fab heavy chain polypeptide of the anti-PSMA antibody.

Embodiment 48 comprises the isolated polypeptide or polypeptide complex of embodiment 46, wherein the linker connects the Fab or Fab′ of the anti-CD28 antibody to the Fab light chain polypeptide of the anti-PSMA antibody.

Embodiment 49 comprises the isolated polypeptide or polypeptide complex of embodiment 46, wherein the linker connects the Fab or Fab′ of the anti-CD28 antibody to the N-term of the Fab heavy chain polypeptide of the anti-PSMA antibody.

Embodiment 50 comprises the isolated polypeptide or polypeptide complex of embodiment 46, wherein the linker connects the Fab or Fab′ of the anti-CD28 antibody to the C-term of the Fab heavy chain polypeptide of the anti-PSMA antibody.

Embodiment 51 comprises the isolated polypeptide or polypeptide complex of embodiment 46, wherein the linker connects the Fab or Fab′ of the anti-CD28 antibody to the N-term of the Fab light chain polypeptide of the anti-PSMA antibody.

Embodiment 52 comprises the isolated polypeptide or polypeptide complex of embodiment 46, wherein the linker connects the Fab or Fab′ of the anti-CD28 antibody to the C-term of the Fab light chain polypeptide of the anti-PSMA antibody.

Embodiment 53 comprises the isolated polypeptide or polypeptide complex of embodiment 46, wherein the linker connects a Fab heavy chain polypeptide of the anti-CD28 antibody to the Fab heavy chain polypeptide of the anti-PSMA antibody.

Embodiment 54 comprises the isolated polypeptide or polypeptide complex of embodiment 46, wherein the linker connects a Fab heavy chain polypeptide of the anti-CD28 antibody to the Fab light chain polypeptide of the anti-PSMA antibody.

Embodiment 55 comprises the isolated polypeptide or polypeptide complex of embodiment 46, wherein the linker connects a Fab heavy chain polypeptide of the anti-CD28 antibody to the N-term of the Fab heavy chain polypeptide of the anti-PSMA antibody.

Embodiment 56 comprises the isolated polypeptide or polypeptide complex of embodiment 46, wherein the linker connects a Fab heavy chain polypeptide of the anti-CD28 antibody to the C-term of the Fab heavy chain polypeptide of the anti-PSMA antibody.

Embodiment 57 comprises the isolated polypeptide or polypeptide complex of embodiment 46, wherein the linker connects a Fab heavy chain polypeptide of the anti-CD28 antibody to the N-term of the Fab light chain polypeptide of the anti-PSMA antibody.

Embodiment 58 comprises the isolated polypeptide or polypeptide complex of embodiment 46, wherein the linker connects a Fab heavy chain polypeptide of the anti-CD28 antibody to the C-term of the Fab light chain polypeptide of the anti-PSMA antibody.

Embodiment 59 comprises the isolated polypeptide or polypeptide complex of embodiment 46, wherein the linker connects a Fab light chain polypeptide of the anti-CD28 antibody to the Fab heavy chain polypeptide of the anti-PSMA antibody.

Embodiment 60 comprises the isolated polypeptide or polypeptide complex of embodiment 46, wherein the linker connects a Fab light chain polypeptide of the anti-CD28 antibody to the Fab light chain polypeptide of the anti-PSMA antibody.

Embodiment 61 comprises the isolated polypeptide or polypeptide complex of embodiment 46, wherein the linker connects a Fab light chain polypeptide of the anti-CD28 antibody to the N-term of the Fab heavy chain polypeptide of the anti-PSMA antibody.

Embodiment 62 comprises the isolated polypeptide or polypeptide complex of embodiment 46, wherein the linker connects a Fab light chain polypeptide of the anti-CD28 antibody to the N-term of the Fab heavy chain polypeptide of the anti-PSMA antibody.

Embodiment 63 comprises the isolated polypeptide or polypeptide complex of embodiment 46, wherein the linker connects a Fab light chain polypeptide of the anti-CD28 antibody to the N-term of the Fab light chain polypeptide of the anti-PSMA antibody.

Embodiment 64 comprises the isolated polypeptide or polypeptide complex of embodiment 46, wherein the linker connects a Fab light chain polypeptide of the anti-CD28 antibody to the C-term of the Fab light chain polypeptide of the anti-PSMA antibody.

Embodiment 65 comprises the isolated polypeptide or polypeptide complex of any one of embodiments 17-24, wherein the anti-CD28 antibody comprises a CrossFab and the anti-PSMA antibody comprises a Fab or Fab′.

Embodiment 66 comprises the isolated polypeptide or polypeptide complex of embodiment 65, wherein the CrossFab and the Fab or Fab′ are connected through a linker and the Fab or Fab′ of the anti-PSMA antibody comprise a Fab heavy chain polypeptide and a Fab light chain polypeptide.

Embodiment 67 comprises the isolated polypeptide or polypeptide complex of embodiment 66, wherein the linker connects the CrossFab to the Fab heavy chain polypeptide of the anti-PSMA antibody.

Embodiment 68 comprises the isolated polypeptide or polypeptide complex of embodiment 66, wherein the linker connects the CrossFab to the Fab light chain polypeptide of the anti-PSMA antibody.

Embodiment 69 comprises the isolated polypeptide or polypeptide complex of embodiment 66, wherein the linker connects the CrossFab to the N-term of the Fab heavy chain polypeptide of the anti-PSMA antibody.

Embodiment 70 comprises the isolated polypeptide or polypeptide complex of embodiment 66, wherein the linker connects the CrossFab to the C-term of the Fab heavy chain polypeptide of the anti-PSMA antibody.

Embodiment 71 comprises the isolated polypeptide or polypeptide complex of embodiment 66, wherein the linker connects the CrossFab to the N-term of the Fab light chain polypeptide of the anti-PSMA antibody.

Embodiment 72 comprises the isolated polypeptide or polypeptide complex of embodiment 66, wherein the linker connects the CrossFab to the C-term of the Fab light chain polypeptide of the anti-PSMA antibody.

Embodiment 73 comprises the isolated polypeptide or polypeptide complex of embodiment 66, wherein the linker connects a Fab heavy chain polypeptide of the CrossFab to the Fab heavy chain polypeptide of the anti-PSMA antibody.

Embodiment 74 comprises the isolated polypeptide or polypeptide complex of embodiment 66, wherein the linker connects a Fab heavy chain polypeptide of the CrossFab to the Fab light chain polypeptide of the anti-PSMA antibody.

Embodiment 75 comprises the isolated polypeptide or polypeptide complex of embodiment 66, wherein the linker connects a Fab heavy chain polypeptide of the CrossFab to the N-term of the Fab heavy chain polypeptide of the anti-PSMA antibody.

Embodiment 76 comprises the isolated polypeptide or polypeptide complex of embodiment 66, wherein the linker connects a Fab heavy chain polypeptide of the CrossFab to the C-term of the Fab heavy chain polypeptide of the anti-PSMA antibody.

Embodiment 77 comprises the isolated polypeptide or polypeptide complex of embodiment 66, wherein the linker connects a Fab heavy chain polypeptide of the Cross-Fab to the N-term of the Fab light chain polypeptide of the anti-PSMA antibody.

Embodiment 78 comprises the isolated polypeptide or polypeptide complex of embodiment 66, wherein the linker connects a Fab heavy chain polypeptide of the CrossFab to the C-term of the Fab light chain polypeptide of the anti-PSMA antibody.

Embodiment 79 comprises the isolated polypeptide or polypeptide complex of embodiment 66, wherein the linker connects a Fab light chain polypeptide of the CrossFab to the Fab heavy chain polypeptide of the anti-PSMA antibody.

Embodiment 80 comprises the isolated polypeptide or polypeptide complex of embodiment 66, wherein the linker connects a Fab light chain polypeptide of the CrossFab to the Fab light chain polypeptide of the anti-PSMA antibody.

Embodiment 81 comprises the isolated polypeptide or polypeptide complex of embodiment 66, wherein the linker connects a Fab light chain polypeptide of the CrossFab to the N-term of the Fab heavy chain polypeptide of the anti-PSMA antibody.

Embodiment 82 comprises the isolated polypeptide or polypeptide complex of embodiment 66, wherein the linker connects a Fab light chain polypeptide of the CrossFab to the C-term of the Fab heavy chain polypeptide of the anti-PSMA antibody.

Embodiment 83 comprises the isolated polypeptide or polypeptide complex of embodiment 66, wherein the linker connects a Fab light chain polypeptide of the CrossFab to the N-term of the Fab light chain polypeptide of the anti-PSMA antibody.

Embodiment 84 comprises the isolated polypeptide or polypeptide complex of embodiment 66, wherein the linker connects a Fab light chain polypeptide of the CrossFab to the C-term of the Fab light chain polypeptide of the anti-PSMA antibody.

Embodiment 85 comprises the isolated polypeptide or polypeptide complex of any one of embodiments 17-20, wherein the anti-CD28 antibody comprises a Fab or Fab′ and the anti-PSMA antibody comprises a CrossFab.

Embodiment 86 comprises the isolated polypeptide or polypeptide complex of any one of embodiments 26-44, 46-64, and 66-84, wherein the linker is at least 5 amino acids in length.

Embodiment 87 comprises the isolated polypeptide or polypeptide complex of any one of embodiments 26-44, 46-64, and 66-84, wherein the linker is no more than 30 amino acids in length.

Embodiment 88 comprises the isolated polypeptide or polypeptide complex of any one of embodiments 26-44,46-64, and 66-84, wherein the linker is at least 5 amino acids and no more than 30 amino acids in length.

Embodiment 89 comprises the isolated polypeptide or polypeptide complex of any one of embodiments 26-44,46-64, and 66-84, wherein the linker is 5 amino acids in length.

Embodiment 90 comprises the isolated polypeptide or polypeptide complex of any one of embodiments 2644, 46-64, and 66-84, wherein the linker is 15 amino acids in length.

Embodiment 91 comprises the isolated polypeptide or polypeptide complex of any one of embodiments 26-44, 46-64, and 66-84, wherein the linker comprises an amino acid sequence of SEQ ID NO: 21 (GGGGSGGGGSGGGGS) or SEQ ID NO: 22 (GGGGS).

Embodiment 92 comprises the isolated polypeptide or polypeptide complex of embodiment 1, wherein the isolated polypeptide or polypeptide complex comprises amino acid sequences with at least 80%, 85%, 90%, 95%, 980/, 99%, or 100% sequence identity to SEQ ID NO: 23 and SEQ ID NO: 24.

Embodiment 93 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 25 and SEQ ID NO: 26.

Embodiment 94 comprises the isolated polypeptide or polypeptide complex of embodiment 1, wherein the isolated polypeptide or poly peptide complex comprises amino acid sequences with at least 80%, 85%, 90%, 95%, 98%, 99%, or 100% sequence identity to SEQ ID NO: 27 and SEQ ID NO: 28.

Embodiment 95 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 29 and SEQ ID NO: 30.

Embodiment 96 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 31 and SEQ ID NO: 32.

Embodiment 97 comprises the isolated polypeptide or polypeptide complex of embodiment 1, wherein the isolated polypeptide or polypeptide complex comprises the amino acid sequences of SEQ ID NO: 25 and SEQ ID NO: 26.

Embodiment 98 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 706, and SEQ ID NO: 707.

Embodiment 99 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 708, SEQ ID NO: 709, and SEQ ID NO: 710.

Embodiment 100 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 711, SEQ ID NO: 712, and SEQ ID NO: 713.

Embodiment 101 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 714, SEQ ID NO: 715, and SEQ ID NO: 716.

Embodiment 102 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 717, SEQ ID NO: 718, and SEQ ID NO: 719.

Embodiment 103 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 720, SEQ ID NO: 721, and SEQ ID NO: 722.

Embodiment 104 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 723, SEQ ID NO: 724, and SEQ ID NO: 725.

Embodiment 105 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 726, SEQ ID NO: 727, and SEQ ID NO: 728.

Embodiment 106 comprises the isolated polypeptide or polypeptide complex of embodiment 1, wherein the isolated polypeptide or polypeptide complex comprises amino acid sequences with at least 80%, 85%, 90%, 95%, 98%, 99%, or 100% sequence identity to SEQ ID NO: 729, SEQ ID NO: 730, and SEQ ID NO: 731.

Embodiment 107 comprises the isolated polypeptide or polypeptide complex of any one of the preceding embodiments, wherein the isolated polypeptide or polypeptide complex is human or humanized.

Embodiment 108 comprises the isolated polypeptide or polypeptide complex of any one of embodiments 2-107, wherein the isolated polypeptide or polypeptide complex comprises a peptide that is linked to the anti-CD28 antibody, wherein the peptide impairs binding of the anti-CD28 antibody to CD28.

Embodiment 109 comprises the isolated polypeptide or polypeptide complex of embodiment 108, wherein the isolated polypeptide or polypeptide complex comprises a configuration according to Formula I: A₂-A₁-L₁-P₁-H₁. wherein A₂ comprises the anti-PSMA antibody, A₁ comprises the anti-CD28 antibody, L, comprises a linking moiety that connects A₁ to P, and is a substrate for a tumor specific protease, P₁ comprises the peptide that impairs binding of the anti-CD28 antibody to CD28, and H₁ comprises a half-life extending molecule.

Embodiment 110 comprises the isolated polypeptide or polypeptide complex of embodiment 109, wherein A₂ further comprises P₂ and L₂, wherein P: comprises a peptide that binds to A₂; and L₂ comprises a linking moiety that connects A₂ to P₂ and is a substrate for a tumor specific protease.

Embodiment 111 comprises the isolated polypeptide or polypeptide complex of embodiment 110, wherein the polypeptide or polypeptide complex is according to Formula Ia: P₂-A₂-A₁-L₁-P₁-H₁

Embodiment 112 comprises the isolated polypeptide or polypeptide complex of any one of embodiments 109-111, wherein the anti-CD28 antibody comprises a scFv and the scFv comprises a scFv heavy chain polypeptide and a scFv light chain polypeptide, and the anti-PSMA antibody comprises a Fab or Fab′ and the Fab or Fab′ comprise a Fab heavy chain polypeptide and a Fab light chain polypeptide.

Embodiment 113 comprises the isolated polypeptide or polypeptide complex of embodiment 112, wherein the Fab heavy chain polypeptide of A₂ is bound to the scFv heavy chain polypeptide of A₁ and L₂ is bound to the Fab light chain polypeptide of A₂.

Embodiment 114 comprises the isolated polypeptide or polypeptide complex of embodiment 112, wherein the Fab light chain polypeptide of A₂ is bound to the scFv heavy chain polypeptide of A₁ and L₂ is bound to the Fab heavy chain polypeptide of A₂.

Embodiment 115 comprises the isolated polypeptide or polypeptide complex of embodiment 112, wherein the Fab heavy chain polypeptide of A₂ is bound to the scFv light chain polypeptide of A₁ and L₂ is bound to the Fab light chain polypeptide of A₂.

Embodiment 116 comprises the isolated polypeptide or polypeptide complex of embodiment 112, wherein the Fab light chain polypeptide of A₂ is bound to the scFv light chain polypeptide of A₁ and L₂ is bound to the Fab heavy chain polypeptide of A₂.

Embodiment 117 comprises the isolated polypeptide or polypeptide complex of any one of embodiments 109-116, wherein P₁ is bound to A₁ through ionic interactions, electrostatic interactions, hydrophobic interactions, Pi-stacking interactions, or H-bonding interactions, or a combination thereof.

Embodiment 118 comprises the isolated polypeptide or polypeptide complex of any one of embodiments 109-117, wherein P, has less than 70% sequence homology to CD28.

Embodiment 119 comprises the isolated polypeptide or polypeptide complex of any one of embodiments 111-118, wherein P₂ impairs binding of A₂ to PSMA.

Embodiment 120 comprises the isolated polypeptide or polypeptide complex of any one of embodiments 111-119, wherein P₂ is bound to A₂ through ionic interactions, electrostatic interactions, hydrophobic interactions, Pi-stacking interactions, or H-bonding interactions, or a combination thereof.

Embodiment 121 comprises the isolated polypeptide or polypeptide complex of any one of embodiments 111-120, wherein P₂ is bound to A₂ at or near an antigen binding site.

Embodiment 122 comprises the isolated polypeptide or polypeptide complex of any one of embodiments 111-121, wherein P₂ has less than 70% sequence homology to PSMA.

Embodiment 123 comprises the isolated polypeptide or polypeptide complex of any one of embodiments 109-122, wherein P₁ or P₂ comprises a peptide sequence of at least 10 amino acids in length.

Embodiment 124 comprises the isolated polypeptide or polypeptide complex of any one of embodiments 109-122, wherein P₁ or P₂ comprises a peptide sequence of at least 10 amino acids in length and no more than 20 amino acids in length.

Embodiment 125 comprises the isolated polypeptide or polypeptide complex of any one of embodiments 109-122, wherein P₁ or P₂ comprises a peptide sequence of at least 16 amino acids in length acids.

Embodiment 126 comprises the isolated polypeptide or polypeptide complex of any one of embodiments 109-122, wherein P₁ or P₂ comprises a peptide sequence of no more than 40 amino in length.

Embodiment 127 comprises the isolated polypeptide or polypeptide complex of any one of embodiments 109-126, wherein P₁ or P₂ comprises at least two cysteine amino acid residues.

Embodiment 128 comprises the isolated polypeptide or polypeptide complex of any one of embodiments 109-127, wherein P₁ or P₂ comprises a cyclic peptide or a linear peptide.

Embodiment 129 comprises the isolated polypeptide or polypeptide complex of any one of embodiments 109-128, wherein P₁ or P₂ comprises a cyclic peptide.

Embodiment 130 comprises the isolated polypeptide or polypeptide complex of any one of embodiments 109-129, wherein P₁ or P₂ comprises a linear peptide.

Embodiment 131 comprises the isolated polypeptide or polypeptide complex of any one of embodiments 109-130, wherein P₁ comprises at least two cysteine amino acid residues.

Embodiment 132 comprises the isolated polypeptide or polypeptide complex of any one of embodiments 109-122, wherein P₁ comprises an amino acid sequence according to any one of SEQ ID NOs: 33 to 106, or 732-813.

Embodiment 133 comprises the isolated polypeptide or polypeptide complex of any one of embodiments 109-122, wherein P₁ comprises the amino acid sequence of SEQ ID NO: 73 or a peptide sequence that has 1, 2, or 3 amino acid substitutions, additions, or deletions relative to the amino acid sequence of SEQ ID NO: 73.

Embodiment 134 comprises the isolated polypeptide or polypeptide complex of any one of embodiments 109-122, wherein P₁ comprises the amino acid sequence of SEQ ID NO: 73.

Embodiment 135 comprises the isolated polypeptide or polypeptide complex of any one of embodiments 109-122, wherein P₁ comprises the amino acid sequence of SEQ ID NO: 103 or a peptide sequence that has 1, 2, or 3 amino acid substitutions, additions, or deletions relative to the amino acid sequence of SEQ ID NO: 103.

Embodiment 136 comprises the isolated polypeptide or polypeptide complex of any one of embodiment 109-122, wherein P₁ comprises the amino acid sequence of SEQ ID NO: 103.

Embodiment 137 comprises the isolated polypeptide or polypeptide complex of any one of embodiments 109-122, wherein P₁ comprises an amino acid sequence according to X₁-X₂-X₃-C-X₄-X₅-X₆ X₇-X₈-X₉-X₁₁)-C-X₁₁-X₂ wherein X₁ is selected from M, I, L, and V; X₂ is selected from D, H, N, A, F, S, T, Y, and V: X₃ is selected from W, L, and F; X₄ is selected from P, A, and L; X₅ is selected from R, T, I, M, S, K, L, V, W, F, A, P, and D; X₆ is selected from E, D, Y, H, S, F, A, N, T, I, P, and V: X₇ is selected from L, M, R, S, Q, and H; X₈ is selected from W and Q; X₀ is selected from H, N, D, A, S, Y, T, F, V, L, and I; X₁₀ is selected from E. V. L, D, Y, R, Q, H, F, K, A, M, and N; X₁₁ is selected from F, Y, L, W, and V; and X₂ is selected from N, A, F, S, Y, H, D, T, and L.

Embodiment 138 comprises the isolated polypeptide or polypeptide complex of embodiment 137, wherein X₁ is selected from M, I, and L; X₂ is selected from D, H, N, and A; X₃ is W; X₄ is P; X₅ is selected from R, T, I, M, S, and K; X₆ is selected from E, D, Y, H, S, and F; X₇ is selected from L, M, and R X₈ is W; X₉ is selected from H, N, D, A, S, and V; X₁₁ is selected from E, V, L, D, and H: X₁₁ is selected from F. Y, and L; and X₁₂ is selected from N, A, F, S, and Y.

Embodiment 139 comprises the isolated polypeptide or polypeptide complex of embodiment 137, wherein X₁ is M; X₂ is selected from D and H: X₃ is W: X₄ is P; X₅ is selected from R, T, and I; X₆ is selected from E, D, and Y; X₇ is selected from L, M, and R: X₈ is W; X₈ is selected from H, N, D, and V: X₁₀ is selected from E, V, L, D, and H: X₁₁ is F; and X₁₂ is selected from N, A, and F.

Embodiment 140 comprises the isolated polypeptide or polypeptide complex of any one of embodiments 109-122, wherein P₁ₐ comprises an amino acid sequence according to SEQ ID NO: 41 or an amino acid sequence that has 1, 2, or 3 amino acid mutations, substitutions, or deletions relative to SEQ ID NO: 41.

Embodiment 141 comprises the isolated polypeptide or polypeptide complex of any one of embodiments 109-122, wherein P₁ comprises an amino acid sequence according to SEQ ID NO: 41.

Embodiment 142 comprises the isolated polypeptide or polypeptide complex of any one of embodiments 109-122, wherein P₁ comprises an amino acid sequence selected from any one of SEQ ID NOs: 41, 73, 103, and 179-603.

Embodiment 143 comprises the isolated polypeptide or polypeptide complex of any one of embodiments 109-122, wherein P₁ comprises an amino acid sequence according to: J₁-J₂-W-C-J₃-J₄-J₅-J₆-J₇-J₈-J₉-C-J₁₀-J₁₁, wherein
J₁ is selected from A, R, N, D, Q, E, G, H, I, L, K, F, P, S, T, W, Y, and V;
J₂ is selected from D, P, and Y;
J₃ is selected from P and Q:
J₄ is selected from R, L, and I:
J₅ is selected from D, H, Y, and I:
J₆ is selected from L, S, and R;
J₇ is selected from W and G;
J₈ is selected from V, A, D, H, L, and N:
J₉ is selected from H, L, E, and D;
J₁₀ is selected from F, Y, and L; and
J₁₁ is selected from A, N, H, and T.

Embodiment 144 comprises the isolated polypeptide or polypeptide complex of embodiment 143, wherein: J₁ is selected from A, R, N, D, Q, E, G, H, K, F, P, S, T, W, and Y.

Embodiment 145 comprises the isolated polypeptide or polypeptide complex of embodiment 143, wherein:
J₁ is selected from R, Q, E, H, I, L, K, F, T, W, Y, and V;
J₂ is D;
J₃ is P:
J₄ is R;
J₅ is D;
J₁ is L;
J₇ is W;
J₈ is V;
J₉ is H;
J₁₀ is F; and
J₁₁ is A.

Embodiment 146 comprises the isolated polypeptide or polypeptide complex of embodiment 143, wherein:
J₁ is selected from R, Q, E, L, K, T, and W;
J₂ is D;
J₃ is P;
J₄ is R;
J₅ is D;
J₆ is L;
J₇ is W;
J₈ is V;
J₈ is H;
J₁₀ is F; and
J₁₁ is A.

Embodiment 147 comprises the isolated polypeptide or polypeptide complex of embodiment 143, wherein:
J₁ is L or I;
J₂ is D or Y;
J₃ is P;
J₄ is R, L₁ or I;
J₅ is H, Y, or D;
J₆ is L or R
J₇ is W:
J₈ is A, D, H, or N;
J₈ is L, E, or D;
J₁₀ is F or Y; and
J₁₁ is N or H.

Embodiment 148 comprises the isolated polypeptide or polypeptide complex of embodiment 143, wherein:
J₁ is L or I;
J₂ is D or Y;
J₃ is P;
J₄ is R or I;
J₅ is H, Y, or D;
J₆ is L or R:
J₇ is W;
J₈ is A, D, or N;
J₉ is L, E, or D;
J₁₀ is F or Y; and
J₁₁ is N.

Embodiment 149 comprises the isolated polypeptide or polypeptide complex of any one of embodiments 109-122, wherein P₁ comprises an amino acid sequence according to: Z₁-C-Z₂-Z₃-Z₄-Z₈-Z₆-Z₇-Z₈-Z₉-Z₁₀-Z₁₁-C-Z₁₂, wherein:
Z₁ is selected from Y, H, and A;
Z₂ is selected from D, E, A, Q, S, V, M, H, L, F, T, W, and N;
Z₃ is selected from G, E, S, and A;
Z₄ is selected from H, L, W, and A;
Z₅ is selected from L, S, A, T, I, V, M, R, E, D, F, H, K, Q, N, Y, W, and G:
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, Q, and A;
Z₉ is selected from M, I, L, R, V, K, F, Q, Y, W, and A;
Z₁₀ is selected from A, Q, S, W, E, L, G, D, T, M, N, V, H, R, Y, K, P, F, and I;
Z₁₁ is selected from K, Q, N, H, E, P, and A; and
Z₁₂ is selected from L, M, I, F, V, Y, Q, D, T, and A.

Embodiment 150 comprises the isolated polypeptide or polypeptide complex of embodiment 149, wherein:
Z₁ is selected from Y and H;
Z₂ is selected from D, E, A, Q, S, V, M, H, L, F, T, W, and N;
Z₃ is selected from G, E, and S;
Z₄ is selected from H, L, and W;
Z₅ is selected from L, S, A, T, I, V, M, R, E, D, F, H, K, Q, N, Y, W, and G;
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, and Q;
Z₉ is selected from M, I, L, R, V, K, F, Q, Y, and W;
Z₁₀ is selected from A, Q, S, W, E, L, G, D, T, M, N, V, H, R, Y, K, P, F, and I;
Z₁₁ is selected from K, Q, N, H, E, and P; and
Z₁₂ is selected from L, M, I, F, V, Y, Q, D, and T.

Embodiment 151 comprises the isolated polypeptide or polypeptide complex of embodiment 149 or 150, wherein:
Z₁ is selected from Y and H:
Z₂ is selected from D, E, A, Q, S, V, M, H, L, F, T, W, and N;
Z₃ is selected from G, E, and S;
Z₄ is selected from H, L, and W;
Z₅ is selected from L, S, A, T, I, V, M, R, E, D, F, H, K, Q, N, Y, W, and G:
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, and Q.
Z₉ is selected from M, I, L, R, V, K, F, Q, Y, and W;
Z₁₀ is selected from A, Q, S, W, E, L, G, D, T, M, N, V, H, R, Y, K, P, F, and I;
Z₁₁ is selected from K, Q, N, H, E, and P; and
Z₁₂ is selected from L, M, I, F, V, Y, Q, D, and T.

Embodiment 152 comprises the isolated polypeptide or polypeptide complex of any one of embodiments 149-151, wherein:
Z₁ is Y;
Z₂ is selected from D, E, A, and Q:
Z₃ is G;
Z₄ is H;
Z₅ is selected from L, S, A, T, I, V, and M:
Z₆ is selected from P, A, E, Q, S, L, W, G, V, and D:
Z₇ is selected from E, Q, I, M, V, A, and L;
Z₈ is selected from Y and H:
Z₉ is selected from M, I, and L;
Z₁₀ is selected from A, Q, S, W, E, L, G, D, T, M, and N;
Z₁₁ is K; and
Z₁₂ is selected from L and M.

Embodiment 153 comprises the isolated polypeptide or polypeptide complex of any one of embodiments 149-152, wherein:
Z₁ is Y;
Z₂ is selected from D, E, and A:
Z₃ is G;
Z₄ is H;
Z₅ is selected from L, S, A, and T;
Z₆ is selected from P, A, E, Q, S, L, and W;
Z₇ is selected from E, Q, I, and M;
Z₈ is Y;
Z₉ is selected from M, I, and L;
Z₁₀ is selected from A, Q, S, W, E, L, G, and D;
Z₁₁ is K; and
Z₁₂ is L.

Embodiment 154 comprises the isolated polypeptide or polypeptide complex of any one of embodiments 149-153, wherein:
Z₁ is Y;
Z₂ is selected from D and E;
Z₃ is G;
Z₄ is H;
Z₅ is selected from L, S, and A:
Z₆ is selected from P, A, E, Q, S, and L;
Z₇ is selected from E, Q, and I;
Z₈ is Y:
Z₉ is selected from M, I, and L;
Z₁₀ is selected from A, Q, S, W, E, L, G, and D;
Z₁₁ is K; and
Z₁₂ is L.

Embodiment 155 comprises the isolated polypeptide or polypeptide complex of embodiment 149, wherein P₁ comprises the amino acid sequence of SEQ ID NO: 784.

Embodiment 156 comprises the isolated polypeptide or polypeptide complex of embodiment 149, wherein P₁ comprises the amino acid sequence of SEQ ID NO: 744.

Embodiment 157 comprises the isolated polypeptide or polypeptide complex of embodiment 149, wherein P, consists of the amino acid sequence of SEQ ID NO: 744.

Embodiment 158 comprises the isolated polypeptide or polypeptide complex of embodiment 149, wherein P₁ comprises an amino acid sequence according to any one of SEQ ID NOs: 774-793.

Embodiment 159 comprises the isolated polypeptide or polypeptide complex of embodiment 149, wherein the peptide comprises an amino acid sequence according to any one of SEQ ID NOs: 764-773.

Embodiment 160 comprises the isolated polypeptide or polypeptide complex of any one of embodiments 109-122, wherein P₁ comprises an amino acid sequence according to: U₁-C-U-Ur-U₄-U₅-U₆-U₇-U₈-U₉-U₁₀-U₁₁-C-U₁₂, wherein:
U₁ is selected from T, S, L, M, W, Q, P, R G, N, I, Y, D, E, V, H, and A;
U₂ is selected from A, S, T, K, E, R, G, N, W, and D;
U₃ is selected from A, E, P, D, G, W, Q, V, and F;
U₄ is selected from G, V, F, D, E, R, and A;
U₅ is selected from A, F, V, S, L, P, and M;
U₆ is selected from T, S, H, M, A, N, P, G, Q, D, Y, V, L, I, E, K, F, and R:
U₇ is selected from Y, W, F, L, N, T, Q, I, and A:
U₈ is selected from P, S, T, A, E, K, Q, N, V, G, H, L, D, Y, I, M, and R;
U₉ is selected from W, L, and A;
U₁₀ is selected from P, S, R, L, E, and A;
U₁₁ is selected from L, A, T, M, V, W, Y, E, Q, I, F, S, P, K, R, D, H, N, and G; and
U₁₂ is selected from L, F, V, M, I, Y, E, W, and A.

Embodiment 161 comprises the isolated polypeptide or polypeptide complex of embodiment 160, wherein:
U₁ is selected from T, S, L, M, W, Q, P, R, G, N, I, Y, D, E, V, and H;
U₂ is selected from A, S, T, K, E, R, G, N, W, and D;
U₃ is selected from A, E, P, D, G, W, Q, V, and F;
U₄ is selected from G, V, F, D, E, and R;
U₅ is selected from A, F, V, S, L, P, and M;
U₆ is selected from T, S, H, M, A, N, P, G, Q, D, Y, V, L, I, E, K, F, and R;
U₇ is selected from Y, W, F, L, N, T, Q, and I;
U₈ is selected from P, S, T, A, E, K, Q, N, V, G, H, L, D, Y, I, M, and R:
U₉ is selected from W, L, and A;
U₁₀ is selected from P, S, R, L, and E;
U₁₁ is selected from L, A, T, M, V, W, Y, E, Q, I, F, S, P, K, R, D, H, N, and G; and
U₁₂ is selected from L, F, V, M, I, Y, E, and W.

Embodiment 162 comprises the isolated polypeptide or polypeptide complex of embodiment 160 or 161, wherein:
U₁ is selected from T, S, L, M, and W;
U₂ is selected from A, S, and T;
U₃ is selected from A and E;
U₄ is selected from G, V, and F;
U₅ is selected from A, F, and V;
U₆ is selected from T, S, H, M, A, and N;
U₇ is selected from Y, W, and F;
U₈ is selected from P, S, T, A, E, K, Q, N, and V:
U₉ is selected from W and L;
U₁₀ is selected from P and S;
U₁₁ is selected from L, A, T, M, V, W, Y, E, Q, I, F, and S; and
U₁₂ is selected from L, F, V, and M.

Embodiment 163 comprises the isolated polypeptide or polypeptide complex of any one of embodiments 160 to 162, wherein:
U₁ is selected from T and S;
U₂ is selected from A and S;
U₃ is A:
U₄ is selected from G and V;
U₅ is selected from A and F;
U₆ is selected from T, S, and H:
U₇ is selected from Y and W;
U₈ is selected from P, S, T, and A;
U₉ is W;
U₁₀ is P;
U₁₁ is selected from L, A, T, M, V, W, and Y; and
U₁₂ is selected from L and F.

Embodiment 164 comprises the isolated polypeptide or polypeptide complex of embodiment 160, wherein P₁ comprises the amino acid sequence of SEQ ID NO: 749.

Embodiment 165 comprises the isolated polypeptide or polypeptide complex of embodiment 160, wherein P₁ comprises the amino acid sequence of SEQ ID NO: 749.

Embodiment 166 comprises the isolated polypeptide or polypeptide complex of embodiment 160, wherein P₁ comprises an amino acid sequence according to any one of SEQ ID NOs: 756-763.

Embodiment 167 comprises the isolated polypeptide or polypeptide complex of embodiment 160, wherein P₁ comprises an amino acid sequence according to any one of SEQ ID NOs: 794-813.

Embodiment 168 comprises the isolated polypeptide or polypeptide complex of embodiment 160, wherein P₁ comprises an amino acid sequence according to any one of SEQ ID NOs: 732-2002.

Embodiment 169 comprises the isolated polypeptide or polypeptide complex of any one of embodiments 110-168, wherein P₂ comprises the amino acid sequence of any one of SEQ ID NOs: 2003-4329.

Embodiment 170 comprises the isolated polypeptide or polypeptide complex of any one of embodiments 110-168, wherein P₂ comprises the amino acid sequence of SEQ ID NO: 2025.

Embodiment 171 comprises the isolated polypeptide or polypeptide complex of any one of embodiments 110-168, wherein P₂ consists of the amino acid sequence of SEQ ID NO: 2025.

Embodiment 172 comprises the isolated polypeptide or polypeptide complex of any one of embodiments 110-168, wherein P₂ comprises the amino acid sequence according to any one of SEQ ID NOs: 2087-2094.

Embodiment 173 comprises the isolated polypeptide or polypeptide complex of any one of embodiments 110-168, wherein P₂ comprises the amino acid sequence of SEQ ID NO: 2026.

Embodiment 174 comprises the isolated polypeptide or polypeptide complex of any one of embodiments 110-168, wherein P₂ consists of the amino acid sequence of SEQ ID NO: 2026.

Embodiment 175 comprises the isolated polypeptide or polypeptide complex of any one of embodiments 110-168, wherein P₂ comprises the amino acid sequence according to any one of SEQ ID NOs: 2097-2106.

Embodiment 176 comprises the isolated polypeptide or polypeptide complex of any one of embodiments 110-168, wherein P₂ comprises the amino acid sequence according to any one of SEQ ID NOs: 2107-2133.

Embodiment 177 comprises the isolated polypeptide or polypeptide complex of any one of embodiments 110-168, wherein P₂ comprises the amin acid sequence of SEQ ID NO: 2120.

Embodiment 178 comprises the isolated polypeptide or polypeptide complex of any one of embodiments 110-168, wherein P₂ comprises an amino acid sequence according to B₁-B₂-B₃-B₄-C-B₅-P-B₆-W-B₇-C-B₈-B₉-B₁₀ (SEQ ID NO: 4470), wherein:
B₁ is selected from V, E, L, D, I, G, M, S, P, T, A, F, W, Y, Q, H, N, K, and R;
B₂ is selected from E, V, D, T, S, L, G, P, A, M, I, Q, H, F, Y, N, W, R, and K;
B₃ is selected from K, P, R, I, N, H, V, M, A, L, Q, T, S, G, F, Y, E, W, and D;
B₄ is selected from W, L, M, R, V, Y, A, K, I, S, Q, F, H, E, T, N, G, and D;
B₅ is selected from I, V, T, K, R, E, S, Q, M, L, F, A, N, and H;
B₆ is selected from A, E, S, P, Q, T, L, D, M, V, R, K, N, I, H, W, Y, F, and G;
B₇ is selected from M. L, I, A, V, F, G, and K:
B₈ is selected from E, S, T, A, V, D, Q, L, N, I, M, Y, H, F, W, G, K, R, and P;
B₉ is selected from G, V, A, S, W, E, D, M, T, L, F, H, Q, N, R, I, Y, P, and K; and
B₁₀ is selected from F, L, M, S, I, V, D, Q, T, A, N, Y, W, E, R, H, P, G, and K.

Embodiment 179 comprises the isolated polypeptide or polypeptide complex of any one of embodiments 109-178, wherein L₁ is bound to N-terminus of A₁.

Embodiment 180 comprises the isolated polypeptide or polypeptide complex of any one of embodiments 109-178, wherein L₁ is bound to C-terminus of A₁.

Embodiment 181 comprises the isolated polypeptide or polypeptide complex of any one of embodiments 110-180, wherein L₂ is bound to N-terminus of A₂.

Embodiment 182 comprises the isolated polypeptide or polypeptide complex of any one of embodiments 110-180, wherein L₂ is bound to C-terminus of A₂.

Embodiment 183 comprises the isolated polypeptide or polypeptide complex of any one of embodiments 109-182, wherein L₁ or L₂ is a peptide sequence having at least 5 to no more than 50 amino acids.

Embodiment 184 comprises the isolated polypeptide or polypeptide complex of any one of embodiments 109-182, wherein L₁ or L₂ is a peptide sequence having at least 10 to no more than 30 amino acids.

Embodiment 185 comprises the isolated polypeptide or polypeptide complex of any one of embodiments 109-182, wherein L₁ or L₂ is a peptide sequence having at least 10 amino acids.

Embodiment 186 comprises the isolated polypeptide or polypeptide complex of any one of embodiments 109-182, wherein L₁ or L₂ is a peptide sequence having at least 18 amino acids.

Embodiment 187 comprises the isolated polypeptide or polypeptide complex of any one of embodiments 109-182, wherein L₁ or L₂ is a peptide sequence having at least 26 amino acids.

Embodiment 188 comprises the isolated polypeptide or polypeptide complex of any one of embodiments 109-182, wherein L₁ or L₂has a formula comprising (G₂S)ₙ, wherein n is an integer from 1 to 3 (SEQ ID NO: 605).

Embodiment 189 comprises the isolated polypeptide or polypeptide complex of any one of embodiments 109-182, wherein L, has a formula selected from the group consisting of (G₂S)ₙ, (GS)ₙ, (GSGGS),(SEQ ID NO: 606), (GGGS)ₙ (SEQ ID NO: 607).(GGGGS)D(SEQ ID NO: 608), and (GSSGGS)ₙ (SEQ ID NO: 609), wherein n is an integer of at least 1.

Embodiment 190 comprises the isolated polypeptide or polypeptide complex of any one of embodiments 109-189, wherein P, becomes unbound from A, when L₁ is cleaved by the tumor specific protease thereby exposing A₁ to CD28.

Embodiment 191 comprises the isolated polypeptide or polypeptide complex of any one of embodiments 110-190, wherein P₂ becomes unbound from A: when L₂ is cleaved by the tumor specific protease thereby exposing A₂ to PSMA.

Embodiment 192 comprises the isolated polypeptide or polypeptide complex of any one of embodiments 109-191, wherein the tumor specific protease is selected from the group consisting of a matrix metalloprotease (MMP), serine protease, cysteine protease, threonine protease, and aspartic protease.

Embodiment 193 comprises the isolated polypeptide or polypeptide complex of embodiment 192, wherein the matrix metalloprotease comprises MMP2, MMP7, MMP9, MMP13, or MMP14.

Embodiment 194 comprises the isolated polypeptide or polypeptide complex of embodiment 192, wherein the serine protease comprises matriptase (MTSP1), urokinase, or hepsin.

Embodiment 195 comprises the isolated polypeptide or polypeptide complex of any one of embodiments 109-194, wherein L₁ or L₂ comprises a urokinase cleavable amino acid sequence, a matriptase cleavable amino acid sequence, matrix metalloprotease cleavable amino acid sequence, or a legumain cleavable amino acid sequence.

Embodiment 196 comprises the isolated polypeptide or polypeptide complex of any one of embodiments 109-182, wherein L₁ or L₂ comprises an amino acid sequence according to SEQ ID NO: 110.

Embodiment 197 comprises the isolated polypeptide or polypeptide complex of any one of embodiments 109-182, wherein L₁ or L₂ comprises an amino acid sequence according to any one of SEQ ID NOs: 107-137 and 604.

Embodiment 198 comprises the isolated polypeptide or polypeptide complex of any one of embodiments 109-182, wherein L₁ or L₂ comprises an amino acid sequence of Linker 25 (ISSGLLSGRSDAG) (SEQ ID NO: 132), Linker 26 (AAGLLAPPGGLSGRSDAG) (SEQ ID NO: 133), Linker 27 (SPLGLSGRSDAG) (SEQ ID NO: 134), or Linker 28 (LSGRSDAGSPLGLAG) (SEQ ID NO: 135), or an amino acid sequence that has 1, 2, or 3 amino acid substitutions, additions, or deletions relative to the amino acid sequence of Linker 25, Linker 26, Linker 27, or Linker 28.

Embodiment 199 comprises the isolated polypeptide or polypeptide complex of any one of embodiments 109-198, wherein H₁ comprises a polymer.

Embodiment 200 comprises the isolated polypeptide or polypeptide complex of embodiment 199, wherein the polymer is polyethylene glycol (PEG).

Embodiment 201 comprises the isolated polypeptide or polypeptide complex of any one of embodiments 109-198, wherein H₁ comprises albumin.

Embodiment 202 comprises the isolated polypeptide or polypeptide complex of any one of embodiments 109-198, wherein H₁ comprises an Fc domain.

Embodiment 203 comprises the isolated polypeptide or polypeptide complex of embodiment 201, wherein the albumin is serum albumin.

Embodiment 204 comprises the isolated polypeptide or polypeptide complex of embodiment 201, wherein the albumin is human serum albumin.

Embodiment 205 comprises the isolated polypeptide or polypeptide complex of any one of embodiments 109-198, wherein H₁ comprises a polypeptide, a ligand, or a small molecule.

Embodiment 206 comprises the isolated polypeptide or polypeptide complex of embodiment 205, wherein the polypeptide, the ligand or the small molecule binds serum protein or a fragment thereof, a circulating immunoglobulin or a fragment thereof, or CD35/CR 1.

Embodiment 207 comprises the isolated polypeptide or polypeptide complex of embodiment 206, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 208 comprises the isolated polypeptide or polypeptide complex of embodiment 206, wherein the circulating immunoglobulin molecule comprises IgG1, IgG2, IgG3, IgG4, sigA, IgM or IgD.

Embodiment 209 comprises the isolated polypeptide or polypeptide complex of embodiment 206, wherein the serum protein is albumin.

Embodiment 210 comprises the isolated polypeptide or polypeptide complex of embodiments 206, wherein the polypeptide is an antibody.

Embodiment 211 comprises the isolated polypeptide or polypeptide complex of embodiment 210, wherein the antibody comprises a single domain antibody, a single chain variable fragment, or a Fab.

Embodiment 212 comprises the isolated polypeptide or polypeptide complex of embodiment 211, wherein the single domain antibody comprises a single domain antibody that binds to albumin.

Embodiment 213 comprises the isolated polypeptide or polypeptide complex of embodiments 211 or 212, wherein the single domain antibody is a human or humanized antibody.

Embodiment 214 comprises the isolated polypeptide or polypeptide complex of embodiment 211, wherein the single domain antibody is 645gH1gL1.

Embodiment 215 comprises the isolated polypeptide or polypeptide complex of embodiment 211, wherein the single domain antibody is 645dsgH5gL4.

Embodiment 216 comprises the isolated polypeptide or polypeptide complex of embodiment 211, wherein the single domain antibody is 23-13-A01-sc02.

Embodiment 217 comprises the isolated polypeptide or polypeptide complex of embodiment 211, wherein the single domain antibody is A 10m3 or a fragment thereof.

Embodiment 218 comprises the isolated polypeptide or polypeptide complex of embodiment 211, wherein the single domain antibody is DOM7r-31.

Embodiment 219 comprises the isolated polypeptide or polypeptide complex of embodiment 211, wherein the single domain antibody is DOM7 h-11-15.

Embodiment 220 comprises the isolated polypeptide or polypeptide complex of embodiment 211, wherein the single domain antibody is Alb-1, Alb-8, or Alb-23.

Embodiment 221 comprises the isolated polypeptide or polypeptide complex of embodiment 211, wherein the single domain antibody is 1OE.

Embodiment 222 comprises the isolated polypeptide or polypeptide complex of embodiment 211, wherein the single domain antibody comprises complementarity determining regions (CDRs): HC-CDR1, HC-CDR2, and HC-CDR3, wherein the HC-CDR1. the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140.

Embodiment 223 comprises the isolated polypeptide or polypeptide complex of embodiment 211, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 142, HC-CDR2: SEQ ID NO: 143, and HC-CDR3: SEQ ID NO: 144.

Embodiment 224 comprises the isolated polypeptide or polypeptide complex of embodiment 211, wherein the single domain antibody comprises an amino acid sequence with at least 80%, 85%, 90%, 95%, 99%, or 100% identity to SEQ ID NO: 141.

Embodiment 225 comprises the isolated polypeptide or polypeptide complex of embodiment 211, wherein the single domain antibody comprises an amino acid sequence with at least 80%, 85%, 90%, 95%, 99%, or 100% identity to SEQ ID NO: 146.

Embodiment 226 comprises the isolated polypeptide or polypeptide complex of embodiment 211, wherein the single domain antibody comprises an amino acid sequence with at least 80%, 85%, 900, 95%, 99%, or 100% identity to SEQ ID NO: 145.

Embodiment 227 comprises the isolated polypeptide or polypeptide complex of embodiment 211, wherein the single domain antibody comprises an amino acid sequence with at least 80%, 85%, 90%, 95%, 99%, or 100% identity to SEQ ID NO: 4448.

Embodiment 228 comprises the isolated polypeptide or polypeptide complex of embodiment 211, wherein the single domain antibody is SA21.

Embodiment 229 comprises the isolated polypeptide or polypeptide complex of any one of the preceding embodiments, wherein the polypeptide or polypeptide complex comprises a modified amino acid, a non-natural amino acid, a modified non-natural amino acid, or a combination thereof.

Embodiment 230 comprises the isolated polypeptide or polypeptide complex of embodiment 229, wherein the modified amino acid or modified non-natural amino acid comprises a post-translational modification.

Embodiment 231 comprises the isolated polypeptide or polypeptide complex of any one of embodiments 109-230, wherein H₁ comprises a linking moiety (L₃) that connects H₁ to P₁

Embodiment 232 comprises the isolated polypeptide or polypeptide complex of embodiment 231, wherein L₃ is a peptide sequence having at least 5 to no more than 50 amino acids.

Embodiment 233 comprises the isolated polypeptide or polypeptide complex of embodiment 231, wherein L₃ is a peptide sequence having at least 10 to no more than 30 amino acids.

Embodiment 234 comprises the isolated polypeptide or polypeptide complex of embodiment 231, wherein L₃ is a peptide sequence having at least 10 amino acids.

Embodiment 235 comprises the isolated polypeptide or polypeptide complex of embodiment 231, wherein L₃ is a peptide sequence having at least 18 amino acids.

Embodiment 236 comprises the isolated polypeptide or polypeptide complex of embodiment 231, wherein L₃ is a peptide sequence having at least 26 amino acids.

Embodiment 237 comprises the isolated polypeptide or polypeptide complex of embodiment 231, wherein L₃ has a formula selected from the group consisting of (G₂S)ₙ, (GS)ₙ, (GSGGS),(SEQ ID NO: 606), (GGGS)ₙ (SEQ ID NO: 607).(GGGGS),(SEQ ID NO: 608), and (GSSGGS),(SEQ ID NO: 609), wherein n is an integer of at least 1.

Embodiment 238 comprises the isolated polypeptide or polypeptide complex of embodiment 231, wherein L; comprises an amino acid sequence according to GGGGSGGGSGG (SEQ ID NO: 610).

Embodiment 239 comprises the isolated polypeptide or polypeptide complex of embodiment 1, wherein the polypeptide or polypeptide complex comprises amino acid sequences with at least 95% sequence identity to SEQ ID NOs: 147 and 148.

Embodiment 240 comprises the isolated polypeptide or polypeptide complex of embodiment 1, wherein the polypeptide or polypeptide complex comprises amino acid sequences with at least 95% sequence identity to SEQ ID NOs: 149 and 150.

Embodiment 241 comprises the isolated polypeptide or polypeptide complex of embodiment 1, wherein the polypeptide or polypeptide complex comprises amino acid sequences with at least 95% sequence identity to SEQ ID NOs: 151 and 152.

Embodiment 242 comprises the isolated polypeptide or polypeptide complex of embodiment 1, wherein the polypeptide or polypeptide complex comprises amino acid sequences with at least 95% sequence identity to SEQ ID NOs: 153 and 154.

Embodiment 243 comprises the isolated polypeptide or polypeptide complex of embodiment 1, wherein the polypeptide or polypeptide complex comprises amino acid sequences with at least 95% sequence identity to SEQ ID NOs: 155 and 156.

Embodiment 244 comprises the isolated polypeptide or polypeptide complex of embodiment 1, wherein the polypeptide or polypeptide complex comprises amino acid sequences with at least 95% sequence identity to SEQ ID NOs: 157 and 158.

Embodiment 245 comprises the isolated polypeptide or polypeptide complex of embodiment 1, wherein the polypeptide or polypeptide complex comprises amino acid sequences with at least 95% sequence identity to SEQ ID NOs: 159 and 160.

Embodiment 246 comprises the isolated polypeptide or polypeptide complex of embodiment 1, wherein the polypeptide or polypeptide complex comprises the amino acid sequences of SEQ ID NO: 159 and SEQ ID NO: 160.

Embodiment 247 comprises the isolated polypeptide or polypeptide complex of embodiment 1, wherein the polypeptide or polypeptide complex comprises amino acid sequences with at least 95% sequence identity to SEQ ID NOs: 161 and 162.

Embodiment 248 comprises the isolated polypeptide or polypeptide complex of embodiment 1, wherein the polypeptide or polypeptide complex comprises amino acid sequences with at least 95% sequence identity to SEQ ID NOs: 163 and 164.

Embodiment 249 comprises the isolated polypeptide or polypeptide complex of embodiment 1, wherein the polypeptide or polypeptide complex comprises amino acid sequences with at least 95% sequence identity to SEQ ID NOs: 165 and 166.

Embodiment 250 comprises the isolated polypeptide or polypeptide complex of embodiment 1, wherein the polypeptide or polypeptide complex comprises amino acid sequences with at least 95% sequence identity to SEQ ID NOs: 167 and 168.

Embodiment 251 comprises the isolated polypeptide or polypeptide complex of embodiment 1, wherein the polypeptide or polypeptide complex comprises amino acid sequences with at least 95% sequence identity to SEQ ID NOs: 169 and 170.

Embodiment 252 comprises the isolated polypeptide or polypeptide complex of embodiment 1, wherein the polypeptide or polypeptide complex comprises amino acid sequences with at least 95% sequence identity to SEQ ID NOs: 171 and 172.

Embodiment 253 comprises the isolated polypeptide or polypeptide complex of embodiment 1, wherein the polypeptide or polypeptide complex comprises the amino acid sequences of SEQ ID NO: 171 and SEQ ID NO: 172.

Embodiment 254 comprises the isolated polypeptide or polypeptide complex of embodiment 1, wherein the polypeptide or polypeptide complex comprises amino acid sequences with at least 95% sequence identity to SEQ ID NOs: 173 and 174.

Embodiment 255 comprises the isolated polypeptide or polypeptide complex of embodiment 1, wherein the polypeptide or polypeptide complex comprises amino acid sequences with at least 95% sequence identity to SEQ ID NOs: 175 and 176.

Embodiment 256 comprises the isolated polypeptide or polypeptide complex of embodiment 1, wherein the polypeptide or polypeptide complex comprises amino acid sequences with at least 95% sequence identity to SEQ ID NOs: 177 and 178.

Embodiment 257 comprises the isolated polypeptide or polypeptide complex of embodiment 108, wherein the polypeptide or polypeptide complex comprises amino acid sequences with at least 80%, 85%, 90%, 95%, 99%, or 100% sequence identity to SEQ ID NO: 4330 and 4331.

Embodiment 258 comprises the isolated polypeptide or polypeptide complex of embodiment 108, wherein the polypeptide or polypeptide complex comprises amino acid sequences with at least 80%, 85%, 90%, 95%, 99%, or 100% sequence identity to SEQ ID NO: 4332 and 4333.

Embodiment 259 comprises the isolated polypeptide or polypeptide complex of embodiment 108, wherein the polypeptide or polypeptide complex comprises amino acid sequences with at least 80%, 85%, 90%, 95%, 99%, or 100% sequence identity to SEQ ID NO: 4334 and 4335.

Embodiment 260 comprises the isolated polypeptide or polypeptide complex of embodiment 108, wherein the polypeptide or polypeptide complex comprises amino acid sequences with at least 80%, 85%, 90%, 95%, 99%, or 100% sequence identity to SEQ ID NO: 4336 and 4337.

Embodiment 261 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4338 and 4339.

Embodiment 262 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4340 and 4341.

Embodiment 263 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4342 and 4343.

Embodiment 264 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4344 and 4345.

Embodiment 265 comprises the isolated polypeptide or polypeptide complex of embodiment 110, wherein the polypeptide or poly peptide complex comprises amino acid sequences with at least 80%, 85%, 90%, 95%, 99%, or 100% sequence identity to SEQ ID NO: 4346 and 4347.

Embodiment 266 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4348 and 4349.

Embodiment 267 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4350 and 4351.

Embodiment 268 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4352 and 4353.

Embodiment 269 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4354 and 4355.

Embodiment 270 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4356 and 4357.

Embodiment 271 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4358 and 4359.

Embodiment 272 comprises the isolated polypeptide or polypeptide complex of embodiment 110, wherein the polypeptide or polypeptide complex comprises amino acid sequences with at least 80%, 85%, 90% 95%, 99%, or 100% sequence identity to SEQ ID NO: 4360 and 4361.

Embodiment 273 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4362 and 4363.

Embodiment 274 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4364 and 4365.

Embodiment 275 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4366 and 4367.

Embodiment 276 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4368 and 4369.

Embodiment 277 comprises the isolated polypeptide or polypeptide complex of embodiment 110, wherein the polypeptide or poly peptide complex comprises amino acid sequences with at least 80%, 85%, 90%, 95%, 99%, or 100% sequence identity to SEQ ID NO: 4370 and 4371.

Embodiment 278 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4372 and 4373.

Embodiment 279 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4374 and 4375.

Embodiment 280 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4376 and 4377.

Embodiment 281 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4378 and 4379.

Embodiment 282 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4380 and 4381.

Embodiment 283 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4382 and 4383.

Embodiment 284 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4384 and 4385.

Embodiment 285 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4386 and 4387.

Embodiment 286 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4388 and 4389.

Embodiment 287 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4390 and 4391.

Embodiment 288 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4392 and 4393.

Embodiment 289 comprises the isolated polypeptide or polypeptide complex of embodiment 110, wherein the polypeptide or poly peptide complex comprises amino acid sequences with at least 80%, 85%, 90%, 95%, 99%, or 100% sequence identity to SEQ ID NO: 4394 and 4395.

Embodiment 290 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4396 and 4397.

Embodiment 291 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4398 and 4399.

Embodiment 292 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4400 and 4401.

Embodiment 293 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4402 and 4403.

Embodiment 294 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4404 and 4405.

Embodiment 295 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4406 and 4407.

Embodiment 296 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4408 and 4409.

Embodiment 297 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4410 and 4411.

Embodiment 298 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4412 and 4413.

Embodiment 299 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4414 and 4415.

Embodiment 300 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4416 and 4417.

Embodiment 301 comprises the isolated polypeptide or polypeptide complex of embodiment 110, wherein the polypeptide or poly peptide complex comprises amino acid sequences with at least 80%, 85%, 90%, 95%, 99%, or 100% sequence identity to SEQ ID NO: 4418 and 4419.

Embodiment 302 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4420 and 4421.

Embodiment 303 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4422 and 4423.

Embodiment 304 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4424 and 4425.

Embodiment 305 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4426 and 4427.

Embodiment 306 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4428 and 4429.

Embodiment 307 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4430 and 4431.

Embodiment 308 comprises the isolated polypeptide or polypeptide complex of embodiment 110, wherein the polypeptide or polypeptide complex comprises amino acid sequences with at least 80%, 85%, 90% 95%, 99%, or 100% sequence identity to SEQ ID NO: 4432 and 4433.

Embodiment 309 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4434 and 4435.

Embodiment 310 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4436 and 4437.

Embodiment 311 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4438 and 4439.

Embodiment 312 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4440 and 4441.

Embodiment 313 comprises the isolated polypeptide or polypeptide complex of embodiment 110, wherein the polypeptide or poly peptide complex comprises amino acid sequences with at least 80%, 85%, 90%, 95%, 99%, or 100% sequence identity to SEQ ID NO: 4442 and 4443.

Embodiment 314 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4444 and 4445.

Embodiment 315 comprises the isolated polypeptide or polypeptide complex of embodiment 110, wherein the polypeptide or polypeptide complex comprises amino acid sequences with at least 80%, 85%, 90%, 95%, 99%, or 100% sequence identity to SEQ ID NO: 4446 and 4447.

Embodiment 316 comprises an isolated polypeptide or polypeptide complex according to Formula II: L₁ₐ-P₁ₐ-H. wherein: L₁ₐ comprises a tumor specific protease-cleaved linking moiety that when uncleaved connects P₁. to an anti-CD28 antibody and the anti-CD28 antibody is connected to an anti-PSMA antibody; P₁ₐ comprises a peptide that impairs binding of the anti-CD28 antibody to CD28, and H₁ₐ comprises a half-life extending molecule.

Embodiment 317 comprises the isolated polypeptide or polypeptide complex of embodiment 316, wherein P₁ₐ when L. is uncleaved impairs binding of the anti-CD28 antibody to CD28.

Embodiment 318 comprises the isolated polypeptide or polypeptide complex of embodiments 316 or 317, wherein the anti-CD28 antibody comprises a human or humanized antibody.

Embodiment 319 comprises the isolated polypeptide or polypeptide complex of any one of embodiments 316-318, wherein H₁ₐ comprises a polymer.

Embodiment 320 comprises the isolated polypeptide or polypeptide complex of embodiment 319, wherein the polymer is polyethylene glycol (PEG).

Embodiment 321 comprises the isolated polypeptide or polypeptide complex of any one of embodiments 316-318, wherein H₁ₐ comprises albumin.

Embodiment 322 comprises the isolated polypeptide or polypeptide complex of any one of embodiments 316-318, wherein H₁ₐ comprises an Fc domain.

Embodiment 323 comprises the isolated polypeptide or polypeptide complex of embodiment 321, wherein the albumin is serum albumin.

Embodiment 324 comprises the isolated polypeptide or polypeptide complex of embodiment 321, wherein the albumin is human serum albumin.

Embodiment 325 comprises the isolated polypeptide or polypeptide complex of any one of embodiments 316-318, wherein H₁ comprises a polypeptide, a ligand, or a small molecule.

Embodiment 326 comprises the isolated polypeptide or polypeptide complex of embodiment 325, wherein the polypeptide, the ligand or the small molecule binds a serum protein or a fragment thereof, a circulating immunoglobulin or a fragment thereof, or CD35/CR1.

Embodiment 327 comprises the isolated polypeptide or polypeptide complex of embodiment 326, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 328 comprises the isolated polypeptide or polypeptide complex of embodiment 326, wherein the circulating immunoglobulin molecule comprises IgG1, IgG2, IgG3, IgG4, s1gA, IgM or IgD.

Embodiment 329 comprises the isolated polypeptide or polypeptide complex of embodiment 326, wherein the serum protein is albumin.

Embodiment 330 comprises the isolated polypeptide or polypeptide complex of embodiment 325, wherein the polypeptide is an antibody.

Embodiment 331 comprises the isolated polypeptide or polypeptide complex of embodiment 330, wherein the antibody comprises a single domain antibody, a single chain variable fragment or a Fab.

Embodiment 332 comprises the isolated polypeptide or polypeptide complex of embodiment 330, wherein the antibody comprises a single domain antibody that binds to albumin.

Embodiment 333 comprises the isolated polypeptide or polypeptide complex of any one of embodiments 330-332, wherein the antibody is a human or humanized antibody.

Embodiment 334 comprises the isolated polypeptide or polypeptide complex of embodiment 331, wherein the single domain antibody is 645gH1gL1.

Embodiment 335 comprises the isolated polypeptide or polypeptide complex of embodiment 331, wherein the single domain antibody is 645dsgH5gL4.

Embodiment 336 comprises the isolated polypeptide or polypeptide complex of embodiment 331, wherein the single domain antibody is 23-13-A01-sc02.

Embodiment 337 comprises the isolated polypeptide or polypeptide complex of embodiment 331, wherein the single domain antibody is A10m3 or a fragment thereof.

Embodiment 338 comprises the isolated polypeptide or polypeptide complex of embodiment 331, wherein the single domain antibody is DOM7r-31.

Embodiment 339 comprises the isolated polypeptide or polypeptide complex of embodiment 331, wherein the single domain antibody is DOM7 h-11-15.

Embodiment 340 comprises the isolated polypeptide or polypeptide complex of embodiment 331, wherein the single domain antibody is Alb-1, Alb-8, or Alb-23.

Embodiment 341 comprises the isolated polypeptide or polypeptide complex of embodiment 331, wherein the single domain antibody is 10E.

Embodiment 342 comprises the isolated polypeptide or polypeptide complex of embodiment 331, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140.

Embodiment 343 comprises the isolated polypeptide or polypeptide complex of embodiment 331, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 142, HC-CDR2: SEQ ID NO: 143, and HC-CDR3: SEQ ID NO: 144.

Embodiment 344 comprises the isolated polypeptide or polypeptide complex of embodiment 331, wherein the single domain antibody comprises an amino acid sequence with at least 80%, 850, 90%, 95%, 99%, or 100% identity to SEQ ID NO: 141.

Embodiment 345 comprises the isolated polypeptide or polypeptide complex of embodiment 331, wherein the single domain antibody comprises an amino acid sequence with at least 80%, 85%, 90%, 95%, 99%, or 100% identity to SEQ ID NO: 146.

Embodiment 346 comprises the isolated polypeptide or polypeptide complex of embodiment 331, wherein the single domain antibody comprises an amino acid sequence with at least 80%, 85%, 90%, 95%, 99%, or 100% identity to SEQ ID NO: 145.

Embodiment 347 comprises the isolated polypeptide or polypeptide complex of embodiment 331, wherein the single domain antibody comprises an amino acid sequence with at least 80%, 85%, 90%, 95%, 99%, or 100% identity to SEQ ID NO: 4448.

Embodiment 348 comprises the isolated polypeptide or polypeptide complex of embodiment 331, wherein the single domain antibody is SA21.

Embodiment 349 comprises the isolated polypeptide or polypeptide complex of any one of any one of embodiments 316-348, wherein H₁ₐ comprises a linking moiety (L₂,) that connects H₁ to P₁.

Embodiment 350 comprises the isolated polypeptide or polypeptide complex of embodiment 349, wherein L₂ₐ is a peptide sequence having at least 5 to no more than 50 amino acids.

Embodiment 351 comprises the isolated polypeptide or polypeptide complex of embodiment 349, wherein L₂, is a peptide sequence having at least 10 to no more than 30 amino acids.

Embodiment 352 comprises the isolated polypeptide or polypeptide complex of embodiment 349, wherein L₂ₐ is a peptide sequence having at least 10 amino acids.

Embodiment 353 comprises the isolated polypeptide or polypeptide complex of embodiment 349, wherein L₂ₐ is a peptide sequence having at least 18 amino acids.

Embodiment 354 comprises the isolated polypeptide or polypeptide complex of embodiment 349, wherein L₂ₐ is a peptide sequence having at least 26 amino acids.

Embodiment 355 comprises the isolated polypeptide or polypeptide complex of embodiment 349, wherein L₂ₐ has a formula selected from the group consisting of (G₂S)ₙ, (GS)ₙ, (GSGGS).(SEQ ID NO: 606), (GGGS)ₙ(SEQ ID NO: 607).(GGGGS)ₙ (SEQ ID NO: 608), and (GSSGGS), (SEQ ID NO: 609). wherein n is an integer of at least 1.

Embodiment 356 comprises the isolated polypeptide or polypeptide complex of embodiment 349, wherein L₂. comprises an amino acid sequence according to SEQ ID NO: 110.

Embodiment 357 comprises the isolated polypeptide or polypeptide complex of any one of embodiments 316-356, wherein P₁. has less than 70% sequence homology to CD28.

Embodiment 358 comprises the isolated polypeptide or polypeptide complex of any one of embodiments 316-357, wherein P₁ₐ comprises a peptide sequence of at least 10 amino acids in length.

Embodiment 359 comprises the isolated polypeptide or polypeptide complex of any one of embodiments 316-358, wherein P₁ₐ comprises a peptide sequence of at least 10 amino acids in length and no more than 20 amino acids in length.

Embodiment 360 comprises the isolated polypeptide or polypeptide complex of any one of embodiments 316-358, wherein P₁ₐ comprises a peptide sequence of at least 16 amino acids in length.

Embodiment 361 comprises the isolated polypeptide or polypeptide complex of any one of embodiments 316-358, wherein P₁ₐ comprises a peptide sequence of no more than 40 amino acids in length.

Embodiment 362 comprises the isolated polypeptide or polypeptide complex of any one of embodiments 316-361, wherein P₁ₐ comprises at least two cysteine amino acid residues.

Embodiment 363 comprises the isolated polypeptide or polypeptide complex of any one of embodiments 316-362, wherein P₁ₐ comprises a cyclic peptide or a linear peptide.

Embodiment 364 comprises the isolated polypeptide or polypeptide complex of any one of embodiments 316-363, wherein P₁ₐ comprises a cyclic peptide.

Embodiment 365 comprises the isolated polypeptide or polypeptide complex of any one of embodiments 316-363, wherein P₁ₐ comprises a linear peptide.

Embodiment 366 comprises the isolated polypeptide or polypeptide complex of any one of embodiments 316-356, wherein P₁ₐ comprises an amino acid sequence according to any one of SEQ ID NOs: 33 to 106, or 732-813.

Embodiment 367 comprises the isolated polypeptide or polypeptide complex of any one of embodiments 316-356, wherein P₁ₐ comprises the amino acid sequence of SEQ ID NO: 73.

Embodiment 368 comprises the isolated polypeptide or polypeptide complex of any one of embodiments 316-356, wherein P₁ₐ comprises the amino acid sequence of SEQ ID NO: 784.

Embodiment 369 comprises the isolated polypeptide or polypeptide complex of any one of embodiments 316-356, wherein P₁ₐ comprises the amino acid sequence of SEQ ID NO: 73 or a peptide sequence that has 1, 2, or 3 amino acid substitutions, additions, or deletions relative to the amino acid sequence of SEQ ID NO: 73.

Embodiment 370 comprises the isolated polypeptide or polypeptide complex of any one of embodiments 316-356, wherein P₁ₐ comprises the amino acid sequence of SEQ ID NO: 73.

Embodiment 371 comprises the isolated polypeptide or polypeptide complex of any one of embodiments 316-356, wherein P₁ₐ comprises the amino acid sequence of SEQ ID NO: 103 or a peptide sequence that has 1, 2, or 3 amino acid substitutions, additions, or deletions relative to the amino acid sequence of SEQ ID NO: 103.

Embodiment 372 comprises the isolated polypeptide or polypeptide complex of any one of embodiments 316-356, wherein P₁ₐ comprises the amino acid sequence of SEQ ID NO: 103.

Embodiment 373 comprises the isolated polypeptide or polypeptide complex of any one of embodiments 316-356, wherein P₁ₐ comprises an amino acid sequence according to X₁-X₂-X₃-C-X₄-X₈-X₆-X₇-X₈-X₉-X₁₀-C-X₁₁-X₁₂ wherein X₁ is selected from M, I, L, and V; X₂ is selected from D, H, N, A, F, S, T, Y, and V; X₃ is selected from W, L, and F; X₄ is selected from P, A, and L; X₃ is selected from R, T, I, M, S, K, L, V, W, F, A, P, and D; X₆ is selected from E, D, Y, H, S, F, A, N, T, I, P, and V; X₁ is selected from L, M, R, S, Q, and H; X₈ is selected from W and Q; X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L.

Embodiment 374 comprises the isolated polypeptide or polypeptide complex of any one of embodiments 373, wherein X₁ is selected from M, I, and L; X₂ is selected from D, H, N, and A; X₃ is W; X₄ is P; X₈ is selected from R, T, I, M, S, and K; X₆ is selected from E, D, Y, H, S, and F; X₁ is selected from L, M, and R; X₉ is W; X₉ is selected from H, N, D, A, S, and V; X₁₀ is selected from E, V, L, D, and H; X₁₁ is selected from F, Y, and L; and X₁₂ is selected from N, A, F, S, and Y.

Embodiment 375 comprises the isolated polypeptide or polypeptide complex of any one of embodiments 373, wherein X₁ is M; X₂ is selected from D and H; X₃ is W; X₄ is P; X₈ is selected from R, T, and I; X₆ is selected from E, D, and Y; X₁ is selected from L, M, and R; X₈ is W; X₉ is selected from H, N, D, and V; X₁₀ is selected from E, V, L, D, and H; X₁₁ is F; and X₁₂ is selected from N. A, and F.

Embodiment 376 comprises the isolated polypeptide or polypeptide complex of any one of embodiments 316-356, wherein P₁ₐ comprises an amino acid sequence according to SEQ ID NO: 41 or an amino acid sequence that has 1, 2, or 3 amino acid mutations, substitutions, or deletions relative to SEQ ID NO: 41.

Embodiment 377 comprises the isolated polypeptide or polypeptide complex of any one of embodiments 316-356, wherein P₁ₐ comprises an amino acid sequence according to SEQ ID NO: 41.

Embodiment 378 comprises the isolated polypeptide or polypeptide complex of any one of embodiments 316-356, wherein P₁ₐ comprises an amino acid sequence according to: X₁-X₂-W-C-X₃-X₄-X₅-X₆-X₇-X₈-X₉-C-X₁₀-X₁₁, wherein
X₁ is selected from A, R, N, D, Q, E, G, H, I, L, K, F, P, S, T, W, Y, and V;
X₂ is selected from D, P, and Y;
X₃ is selected from P and Q;
X₄ is selected from R, L, and I;
X₅ is selected from D, H, Y, and I;
X₆ is selected from L, S, and R;
X₁ is selected from W and G;
X₈ is selected from V, A, D, H, L, and N;
X₉ is selected from H, L, E, and D:
Xin is selected from F, Y, and L; and
X₁₁ is selected from A, N, H, and T.

Embodiment 379 comprises the isolated polypeptide or polypeptide complex of embodiment 378, wherein:
X₁ is selected from A, R, N, D, Q, E, G, H, K, F, P, S, T, W, and Y.

Embodiment 380 comprises the isolated polypeptide or polypeptide complex of embodiment 378, wherein:
X₁ is selected from R, Q, E, H, I, L, K, F, T, W, Y, and V:
X₂ is D:
X₃ is P:
X₄ is R;
X₅ is D;
X₆ is L:
X₇ is W;
X₈ is V;
X₉ is H;
X₁₀ is F; and
X₁₁ is A.

Embodiment 381 comprises the isolated polypeptide or polypeptide complex of embodiment 378, wherein:
X₁ is selected from R, Q, E, L, K, T, and W;
X₂ is D;
X₃ is P;
X₄ is R;
X₅ is D:
X₆ is L;
X₇ is W;
X₈ is V;
X₉ is H;
X₁₀ is F; and
X₁₁ is A.

Embodiment 382 comprises the isolated polypeptide or polypeptide complex of embodiment 378, wherein:
X₁ is L or I;
X₂ is D or Y;
X₃ is P;
X₄ is R, L₁ or I:
X₅ is H. Y. or D;
X₆ is L or R;
X₇ is W;
X₈ is A, D, H, or N;
X₉ is L, E, or D;
X₁₀ is F or Y; and
X₁₁ is Nor H.

Embodiment 383 comprises the isolated polypeptide or polypeptide complex of embodiment 378, wherein:
X₁ is L or I:
X₂ is D or Y;
X₃ is P;
X₄ is R or 1;
X₅ is H, Y, or D;
X₆ is L or R;
X₇ is W;
X₈ is A. D. or N;
X₉ is L, E, or D;
X₁₀ is F or Y; and
X₁₁ is N.

Embodiment 384 comprises the isolated polypeptide or polypeptide complex of any one of embodiments 316-356, wherein P₁ₐ comprises an amino acid sequence according to: Z₁-C-Z₂-Z₃-Z₄-Z₅-Z₆-Z₇-Z₈-Z₉-Z₁₀-Z₁₁-C-Z₁₂, wherein:
Z₁ is selected from Y, H, and A;
Z₂ is selected from D, E, A, Q, S, V, M, H, L, F, T, W, and N:
Z₃ is selected from G, E, S, and A:
Z₄ is selected from H, L, W, and A;
Z₅ is selected from L, S, A, T, I, V, M, R, E, D, F, H, K, Q, N, Y, W, and G:
Z₆ is selected from P, A, E, Q, S, L, W, G, V, D, K, I, M, R, T, F, H, N, and Y:
Z₇ is selected from E, Q, I, M, V, A, L, R, W, P, D, T, K, S, G, H, and N;
Z₈ is selected from Y, H, F, W, N, Q, and A;
Z₉ is selected from M, I, L, R, V, K, F, Q, Y, W, and A;
Z₁₀ is selected from A, Q, S, W, E, L, G, D, T, M, N, V, H, R, Y, K, P, F, and I;
Z₁₁ is selected from K, Q, N, H, E, P, and A; and
Z₁₂ is selected from L, M, I, F, V, Y, Q, D, T, and A.

Embodiment 385 comprises the isolated polypeptide or polypeptide complex of embodiment 384, wherein:
Z₁ is selected from Y and H:
Z₂ is selected from D, E, A, Q, S, V, M, H, L, F, T, W, and N;
Z₃ is selected from G, E, and S;
Z₄ is selected from H, L, and W;
Z₅ is selected from L, S, A, T, I, V, M, R, E, D, F, H, K, Q, N, Y, W, and G:
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, and Q;
Z₉ is selected from M, I, L, R, V, K, F, Q, Y, and W;
Z₁₀ is selected from A, Q, S, W, E, L, G, D, T, M, N, V, H, R, Y, K, P, F, and I;
Z₁₁ is selected from K, Q, N, H, E, and P; and
Z₁₂ is selected from L, M, I, F, V, Y, Q, D, and T.

Embodiment 386 comprises the isolated polypeptide or polypeptide complex of embodiment 384 or 385, wherein:
Z₁ is selected from Y and H;
Z₂ is selected from D. E, A, Q, S, V, M, H, L, F, T, W, and N;
Z₃ is selected from G, E, and S:
Z₄ is selected from H, L, and W;
Z₅ is selected from L, S, A, T, I, V, M, R, E, D, F, H K, Q, N, Y, W, and G;
Z₆ is selected from P, A, E, Q, S, L, W, G, V, D, K, I, M, R, T, F, H, N, and Y:
Z₇ is selected from E, Q, I, M, V, A, L, R, W, P, D, T, K, S, G, H, and N:
Z₈ is selected from Y, H, F, W, N, and Q;
Z₉ is selected from M, I, L, R, V, K, F, Q, Y, and W;
Z₁₀ is selected from A, Q, S, W, E, L, G, D, T, M, N, V, H, R, Y, K, P, F, and I;
Z₁₁ is selected from K, Q, N, H, E, and P; and
Z₁₂ is selected from L, M, I, F, V, Y, Q, D, and T.

Embodiment 387 comprises the isolated polypeptide or polypeptide complex of any one of embodiments 384-386, wherein:
Z₁ is Y:
Z₂ is selected from D, E, A, and Q:
Z₃ is G;
Z₄ is H;
Z₅ is selected from L, S, A, T, I, V, and M;
Z₆ is selected from P, A, E, Q, S, L, W, G, V, and D:
Z₇ is selected from E, Q, I, M, V, A, and L;
Z₈ is selected from Y and H;
Z₉ is selected from M, I, and L;
Z₁₀ is selected from A, Q, S, W, E, L, G, D, T, M, and N;
Z₁₁ is K; and
Z₁₂ is selected from L and M.

Embodiment 388 comprises the isolated polypeptide or polypeptide complex of any one of embodiments 384-387, wherein:
Z₁ is Y;
Z₂ is selected from D. E, and A;
Z₃ is G;
Z₄ is H;
Z₅ is selected from L, S, A, and T;
Z₆ is selected from P, A, E, Q, S, L, and W;
Z₇ is selected from E, Q, I, and M;
Z₈ is Y:
Z₉ is selected from M, I, and L;
Z₁₀ is selected from A, Q, S, W, E, L, G, and D;
Z₁₁ is K; and
Z₁₂ is L.

Embodiment 389 comprises the isolated polypeptide or polypeptide complex of any one of embodiments 384-388, wherein:
Z₁ is Y:
Z₂ is selected from D and E:
Z₃ is G;
Z₄ is H;
Z₅ is selected from L, S, and A:
Z₆ is selected from P, A, E, Q, S, and L;
Z₇ is selected from E, Q, and I;
Z₈ is Y;
Z₉ is selected from M, I, and L;
Z₁₀ is selected from A, Q, S, W, E, L, G, and D:
Z₁₁ is K; and
Z₁₂ is L.

Embodiment 390 comprises the isolated polypeptide or polypeptide complex of any one of embodiments 316-356, wherein P₁ₐ comprises the amino acid sequence of SEQ ID NO: 744.

Embodiment 391 comprises the isolated polypeptide or polypeptide complex of any one of embodiments 316-356, wherein P₁ₐ consists of the amino acid sequence of SEQ ID NO: 744.

Embodiment 392 comprises the isolated polypeptide or polypeptide complex of any one of embodiments 316-356, wherein P₁ₐ comprises an amino acid sequence according to any one of SEQ ID NOs: 774-793.

Embodiment 393 comprises the isolated polypeptide or polypeptide complex of any one of embodiments 316-356, wherein the peptide comprises an amino acid sequence according to any one of SEQ ID NOs: 764-773.

Embodiment 394 comprises the isolated polypeptide or polypeptide complex of any one of embodiments 316-356, wherein P₁ₐ comprises an amino acid sequence according to: U₁-C-U₂-U₃-U₄-U₃-U₆-U₇-U₈-U₉-U₁₀-U₁₁-C-U₁₂, wherein:
U₁ is selected from T, S, L, M, W, Q, P, R, G, N, I, Y, D, E, V, H, and A:
U₂ is selected from A, S, T, K, E, R, G, N, W, and D;
U₃ is selected from A, E, P, D, G, W, Q, V, and F;
U₄ is selected from G, V, F, D, E, R, and A;
U₅ is selected from A, F, V, S, L, P, and M;
U₆ is selected from T, S, H, M, A, N, P, G, Q, D, Y, V, L, I, E, K, F, and R;
U₇ is selected from Y, W, F, L, N, T, Q, I, and A;
U₈ is selected from P, S, T, A, E, K, Q, N, V, G, H, L, D, Y, I, M, and R:
U₉ is selected from W, L, and A;
U₁₀ is selected from P, S, R, L, E, and A;
U₁₁ is selected from L, A, T, M, V, W, Y, E, Q, I, F, S, P, K, R, D, H, N, and G; and
U₁₂ is selected from L, F, V, M, I, Y, E, W, and A.

Embodiment 395 comprises the isolated polypeptide or polypeptide complex of embodiment 394, wherein:
U₁ is selected from T, S, L, M, W, Q, P, R, G, N, I, Y, D, E, V, and H;
U₂ is selected from A, S, T, K, E, R, G, N, W, and D;
U₃ is selected from A, E, P, D, G, W, Q, V, and F;
U₄ is selected from G, V, F, D, E, and R;
U₅ is selected from A, F, V, S, L, P, and M;
U₆ is selected from T, S, H, M, A, N, P, G, Q, D, Y, V, L, I, E, K, F, and R;
U₇ is selected from Y, W, F, L, N, T, Q, and I;
U₈ is selected from P, S, T, A, E, K, Q, N, V, G, H, L, D, Y, I, M, and R;
U₉ is selected from W, L, and A;
U₁₀ is selected from P, S, R, L, and E;
U₁₁ is selected from L, A, T, M, V, W, Y, E, Q, I, F, S, P, K, R, D, H, N, and G; and
U₁₂ is selected from L, F, V, M, I, Y, E, and W.

Embodiment 396 comprises the isolated polypeptide or polypeptide complex of embodiment 394 or 395, wherein:
U₁ is selected from T, S, L, M, and W;
U₂ is selected from A, S, and T;
U₃ is selected from A and E:
U₄ is selected from G, V, and F;
U₅ is selected from A, F, and V:
U₆ is selected from T, S, H, M, A, and N:
U₇ is selected from Y, W, and F:
U₈ is selected from P, S, T, A, E, K, Q, N, and V;
U₉ is selected from W and L;
U₁₀ is selected from P and S;
U₁₁ is selected from L, A, T, M, V, W, Y, E, Q, I, F, and S; and
U₁₂ is selected from L, F, V, and M.

Embodiment 397 comprises the isolated polypeptide or polypeptide complex of any one of embodiments 394-396, wherein:
U₁ is selected from T and S;
U₂ is selected from A and S;
U₃ is A;
U₄ is selected from G and V:
U₅ is selected from A and F;
U₆ is selected from T, S, and H;
U₇ is selected from Y and W;
U₈ is selected from P, S, T, and A;
U₉ is W;
U₁₀ is P;
U₁₁ is selected from L, A, T, M, V, W, and Y; and
U₁₂ is selected from L and F.

Embodiment 398 comprises the isolated polypeptide or polypeptide complex of any one of embodiments 316-356, wherein P₁ₐ comprises the amino acid sequence of SEQ ID NO: 749.

Embodiment 399 comprises the isolated polypeptide or polypeptide complex of any one of embodiments 316-356, wherein P₁ₐ comprises the amino acid sequence of SEQ ID NO: 749.

Embodiment 400 comprises the isolated polypeptide or polypeptide complex of any one of embodiments 316-356, wherein P₁ₐ comprises an amino acid sequence according to any one of SEQ ID NOs: 756-763.

Embodiment 401 comprises the isolated polypeptide or polypeptide complex of any one of embodiments 316-356, wherein P₁ₐ comprises an amino acid sequence according to any one of SEQ ID NOs: 794-813.

Embodiment 402 comprises the isolated polypeptide or polypeptide complex of any one of embodiments 316-356, wherein P₁ₐ comprises an amino acid sequence according to any one of SEQ ID NOs: 732-2002.

Embodiment 403 comprises an isolated polypeptide or polypeptide complex according to Formula III: L_{1b}-P_{1b} wherein: L_{1b} comprises a tumor specific protease-cleaved linking moiety that when uncleaved connects P₁. to an anti-PSMA antibody and the anti-PSMA antibody is connected to an anti-CD3 antibody, and P_{1b} comprises a peptide that impairs binding of the anti-PSMA antibody to PSMA.

Embodiment 404 comprises the isolated polypeptide or polypeptide complex of embodiments 403, wherein P₁ₐ when L₁ₐ is uncleaved impairs binding of the anti-PSMA antibody to PSMA.

Embodiment 405 comprises the isolated polypeptide or polypeptide complex of embodiment 403 or 404, wherein the anti-PSMA antibody comprises a human or humanized antibody.

Embodiment 406 comprises the isolated polypeptide or polypeptide complex of any one of embodiments 403 to 405, wherein P_{1b} has less than 70% sequence homology to PSMA.

Embodiment 407 comprises the isolated polypeptide or polypeptide complex of any one of embodiments 403 to 406, wherein P_{1b} comprises a peptide sequence of at least 10 amino acids in length.

Embodiment 408 comprises the isolated polypeptide or polypeptide complex of any one of embodiments 403 to 406, wherein P_{1b} comprises a peptide sequence of at least 10 amino acids in length and no more than 20 amino acids in length.

Embodiment 409 comprises the isolated polypeptide or polypeptide complex of any one of embodiments 403 to 406, wherein P_{1b} comprises a peptide sequence of at least 16 amino acids in length.

Embodiment 410 comprises the isolated polypeptide or polypeptide complex of any one of embodiments 403 to 409, wherein P_{1b} comprises a peptide sequence of no more than 40 amino acids in length.

Embodiment 411 comprises the isolated polypeptide or polypeptide complex of any one of embodiments 403 to 410, wherein P_{ib} comprises a peptide sequence of no more than 40 amino acids in length.

Embodiment 412 comprises the isolated polypeptide or polypeptide complex of any one of embodiments 403 to 411, wherein P_{1b} comprises at least two cysteine amino acid residues.

Embodiment 413 comprises the isolated polypeptide or polypeptide complex of any one of embodiments 403 to 412, wherein P_{1b} comprises a cyclic peptide or a linear peptide.

Embodiment 414 comprises the isolated polypeptide or polypeptide complex of any one of embodiments 403 to 413, wherein P_{1b} comprises a linear peptide

Embodiment 415 comprises the isolated polypeptide or polypeptide complex of any one of embodiments 403 to 413, wherein:
X₁ is selected from V, E, L, D, I, G, M, S, P, T, A, F, W, Y, Q, H, N, K, and R;
X₂ is selected from E, V, D, T, S, L, G, P, A, M, I, Q, H, F, Y, N, W, R, and K;
X₃ is selected from K, P, R, I, N, V, M, A, L, Q, T, S, G, F, Y, E, W, and D;
X₄ is selected from W, L, M, R, V, Y, A, K, I, S, Q, F, H, E, T, N, G, and D:
X₅ is selected from I. V. T, K, R, E, S, Q, M, L, F, A, N, and H;
X₆ is selected from A, E, S, P, Q, T, L, D, M, V, R, K, N, I, H, W, Y, F, and G;
X₇ is selected from M, L, I, A, V, F, G, and K;
X₈ is selected from E, S, T, A, V, D, Q, L, N, I, M, Y, H, F, W, G, K, R, and P;
X₉ is selected from G, V, A, S, W, E, D, M, T, L, F, H, Q, N, R, I, Y, P, and K; and
X₁₀ is selected from F, L, M, S, 1, V, D, Q, T, A, N, Y, W, E, R, H, P, G, and K.

Embodiment 416 comprises the isolated polypeptide or polypeptide complex of embodiment 403, wherein P_{1b} comprises an amino acid sequence according to SEQ ID NO: 2120.

Embodiment 417 comprises the isolated polypeptide or polypeptide complex of embodiment 403, wherein P_{1b} comprises an amino acid sequence according to SEQ ID NO: 2117.

Embodiment 418 comprises the isolated polypeptide or polypeptide complex of embodiment 403, wherein P₁ₐ comprises an amino acid sequence according to SEQ ID NO: 2025.

Embodiment 419 comprises the isolated polypeptide or polypeptide complex of embodiment 403, wherein P_{1b} comprises an amino acid sequence according to SEQ ID NO: 2026.

Embodiment 420 comprises the isolated polypeptide or polypeptide complex of embodiment 403, wherein P_{1b} comprises an amino acid sequence according to any one of SEQ ID NOs: 2097-2106.

Embodiment 421 comprises the isolated polypeptide or polypeptide complex of embodiment 403, wherein P_{1b} comprises an amino acid sequence according to any one of SEQ ID NOs: 2107-2133.

Embodiment 422 comprises the isolated polypeptide or polypeptide complex of embodiment 403, wherein P₁ₐ comprises an amino acid sequence according to any one of SEQ ID NOs: 2033-4329.

Embodiment 423 comprises the isolated polypeptide or polypeptide complex of any one of embodiments 403 to 422, wherein L_{1b} comprises an amino acid sequence according to any one of SEQ ID NOs: 107-137 and 604.

Embodiment 424 comprises an isolated recombinant nucleic acid molecule encoding the polypeptide or polypeptide complex of any one of embodiments 1-423.

Embodiment 425 comprises a pharmaceutical composition comprising:
(a) the polypeptide or polypeptide complex of any one of embodiments 1-423; and
(b) a pharmaceutically acceptable excipient.

Embodiment 426 comprises a method of treating cancer in a subject in need thereof comprising administering to the subject the polypeptide or polypeptide complex of any one of embodiments 1423.

Embodiment 427 comprises the method of embodiment 426, wherein the polypeptide or polypeptide complex induces T cell mediated cytotoxicity of tumor cells.

Embodiment 428 comprises the method of embodiments 426 or 427, wherein the cancer has cells that express PSMA.

Embodiment 429 comprises the method of embodiments 426 or 427, wherein the cancer is a solid tumor cancer.

Embodiment 430 comprises the method of embodiments 426 or 427, wherein the cancer is lung, breast, cervical, ovarian, colorectal, pancreatic or gastric cancer.

Embodiment 431 comprises the method of any one of embodiments 426-430, wherein the polypeptide or polypeptide complex is administered in combination with an anti-cancer therapy.

Embodiment 432 comprises the method of embodiment 431, wherein the polypeptide or polypeptide complex and the anti-cancer therapy are administered in the same pharmaceutical composition.

Embodiment 433 comprises the method of embodiment 431, wherein the polypeptide or polypeptide complex and the anti-cancer therapy are administered as separate pharmaceutical compositions.

Embodiment 434 comprises the method of any one of embodiments 431-433, wherein the anti-cancer therapy comprises a small molecule, a cell-based therapy, or an antibody-based therapy.

Embodiment 435 comprises the method of embodiment 434, wherein the antibody-based therapy is a T cell engager.

Embodiment 436 comprises the method of embodiment 435, wherein the T cell engager comprises a formula according to: D-L₀-E (Formula 11), wherein D comprises an effector cell binding domain that binds to an effector cell antigen, E comprises a tumor antigen binding domain that binds to a tumor antigen, and L₀ comprises a linker that connects D to E.

Embodiment 437 comprises the method of embodiment 436, wherein D comprises a single chain variable fragment, a single domain antibody, or a Fab fragment.

Embodiment 438 comprises the method of embodiment 437, wherein D comprises the single chain variable fragment.

Embodiment 439 comprises the method of embodiment any one of embodiments 436-438, wherein E comprises a single chain variable fragment, a single domain antibody, or a Fab fragment.

Embodiment 440 comprises the method of embodiment 439, wherein E comprises the Fab fragment.

Embodiment 441 comprises the method of any one of embodiments 436 to 440, wherein the effector cell antigen comprises CD3.

Embodiment 442 comprises the method any one of embodiments 436 to 441, wherein the effector cell binding domain comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F 1-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 443 comprises the method any one of embodiments 436 to 441, wherein the effector cell binding domain comprises an amino acid sequence according to SEQ ID NOs: 611-624.

Embodiment 444 comprises the method of any one of embodiments 436 to 443, wherein the tumor antigen comprises epidermal growth factor receptor (EGFR), prostate-specific membrane antigen (PSMA), or tumor-associated calcium signal transducer 2 (referred to herein after as TROP2).

Embodiment 445 comprises the method of any one of embodiments 436 to 443, wherein the tumor antigen comprises EGFR.

Embodiment 446 comprises the method of embodiment 436, wherein the tumor antigen binding domain comprises an amino acid sequence according to SEQ ID NOs: 629-638.

Embodiment 447 comprises the method of any one of embodiments 436-446, wherein the tumor antigen comprises EGFR, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 632; HC-CDR2: SEQ ID NO: 633; HC-CDR3: SEQ ID NO: 634; and wherein the LC-CDR1, the LC-CDR2, and the LC-CDR3 comprise: LC-CDR1: SEQ ID NO: 629: LC-CDR2: (YAS); and LC-CDR3: SEQ ID NO: 631.

Embodiment 448 comprises the method of any one of embodiments 436 to 444, wherein the tumor antigen comprises EGFR, and the T cell engager comprises amino acid sequences with at least 95% sequence identity according to SEQ ID NOs: 639 and 640.

Embodiment 449 comprises the method of any one of embodiments 436 to 444, wherein the tumor antigen comprises EGFR, and the T cell engager comprises amino acid sequences according to SEQ ID NOs: 641 and 642.

Embodiment 450 comprises the method of any one of embodiments 436 to 444, wherein the tumor antigen comprises TROP2.

Embodiment 451 comprises the method of embodiment 436-444, wherein the tumor antigen comprises TROP2, and the tumor binding domain comprises complementarity determining regions (CDRs): HC-CDR1, HC-CDR2, and HC-CDR3, and LC-CDR1, LC-CDR2, and LC-CDR3, wherein the HC-CDR1, the HC-CDR2, and the HC-CDR3 comprise HC-CDR1: SEQ ID NO: 659; HC-CDR2: SEQ ID NO: 660; HC-CDR3: SEQ ID NO: 661, 662, or 663; and wherein the LC-CDR1, the LC-CDR2, and the LC-CDR3 comprise:LC-CDR1: SEQ ID NO: 664; LC-CDR2: (SAS); and LC-CDR3: SEQ ID NO: 666.

Embodiment 452 comprises the method of any one of embodiments 436-444, wherein the tumor antigen comprises TROP2, and the T cell engager comprises amino acid sequences with at least 95% sequence identity according to SEQ ID NOs: 673 and 674.

Embodiment 453 comprises the method of any one of embodiments 436-444, wherein the tumor antigen comprises TROP2, and the T cell engager comprises amino acid sequences according to SEQ ID NOs: 675 and 676, or SEQ ID NOs: 677 and 678.

Embodiment 454 comprises the method of any one of embodiments 436-444, wherein the tumor antigen binding domain comprises an amino acid sequence according to SEQ ID NOs: 659-668.

Embodiment 455 comprises the method of any one of embodiments 436-444, wherein the tumor antigen comprises PSMA.

Embodiment 456 comprises the method of any one of embodiments 436-444, wherein the tumor antigen binding domain comprises an amino acid sequence according to SEQ ID NOs: 647-654.

Embodiment 457 comprises the method of any one of embodiments 436-444, wherein the tumor antigen comprises PSMA, and the tumor binding domain comprises complementarity determining regions (CDRs): HC-CDR1, HC-CDR2, and HC-CDR3, and LC-CDR1. LC-CDR2, and LC-CDR3, wherein the HC-CDR1, the HC-CDR2, and the HC-CDR3 comprise HC-CDR1: SEQ ID NO: 647; HC-CDR2: SEQ ID NO: 648; HC-CDR3: SEQ ID NO: 649; and wherein the LC-CDR1, the LC-CDR2, and the LC-CDR3 comprise:LC-CDR1: SEQ ID NO: 650; LC-CDR2: SEQ ID NO: 651; and LC-CDR3: SEQ ID NO: 652.

Embodiment 458 comprises the method of any one of embodiments 436-444, wherein the tumor antigen comprises PSMA, and the T cell engager comprises amino acid sequences with at least 95% sequence identity according to SEQ ID NOs: 655 and 656.

Embodiment 459 comprises the method of any one of embodiments 436-458, wherein the T cell engager molecule is selectively activated in tumor microenvironments.

Embodiment 460 comprises the method of embodiment 435, wherein the T cell engager is according to the following subformula: P₃-L₃-D-L₀-E (Formula IIa) wherein D comprises a CD3 binding domain; E comprises the tumor antigen binding domain; L₀ comprises the linker that connects D to E; P₃ comprises a peptide that binds to D and L₃ comprises a linking moiety that connects D to P₃ and is a substrate for a tumor specific protease.

Embodiment 461 comprises the method of embodiment 435, wherein the T cell engager is according to the following subformula: D-L₀-E-L₁,-P₄ (Formula fib) wherein D comprises a CD3 binding domain; E comprises the tumor antigen binding domain: L-comprises the linker that connects D to E: P₄ comprises a peptide that binds to E and L₄ comprises a linking moiety that connects E to P₄ and is a substrate for a tumor specific protease.

Embodiment 462 comprises the method of embodiment 435, wherein the T cell engager is according to the following subformula: P₃-L₃-D-L₀-E-L₄-P₄ (Formula IIc) wherein D comprises a CD3 binding domain; E comprises the tumor antigen binding domain; L₀ comprises the linker that connects D to E; P₃ comprises a peptide that binds to D and L₃ comprises a linking moiety that connects D to P₃ and is a substrate for a tumor specific protease: P₄ comprises a peptide that binds to E and La comprises a linking moiety that connects E to P₄ and is a substrate for a tumor specific protease.

Embodiment 463 comprises the method of any one of embodiments 435-462, wherein the T cell engager comprises H₁.

Embodiment 464 comprises the method of embodiment 463, wherein H₁ comprises a sequence according to SEQ ID NO: 141, SEQ ID NO: 145, SEQ ID NO: 146, or SEQ ID NO: 4448.

Embodiment 465 comprises the method of embodiments 463 or 464, H₁ comprises a single domain antibody.

Embodiment 466 comprises the method of embodiment 465, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140.

Embodiment 467 comprises the method of any one of embodiments 460466, wherein L₃ or L₄ is a peptide sequence having at least 5 to no more than 50 amino acids.

Embodiment 468 comprises the method of any one of embodiments 460466, wherein L₃ or L₄ is a peptide sequence having at least 10 to no more than 30 amino acids.

Embodiment 469 comprises the method of any one of embodiments 460466. wherein L₃ or L₄ is a peptide sequence having at least 10 amino acids.

Embodiment 470 comprises the method of any one of embodiments 460-466, wherein L₃ or L₄ is a peptide sequence having at least 18 amino acids.

Embodiment 471 comprises the method of any one of embodiments 460466, wherein L₃ or L₄ is a peptide sequence having at least 26 amino acids.

Embodiment 472 comprises the method of any one of embodiments 460466, wherein L₃ or L₄has a formula comprising (G₂S)ₙ, wherein n is an integer from 1 to 3 (SEQ ID NO: 605).

Embodiment 473 comprises the method of any one of embodiments 460466. wherein L₃ or L₄ has a formula comprising (G₂S)ₙ, wherein n is an integer of at least 1.

Embodiment 474 comprises the method of any one of embodiments 460466, wherein L₃ or L₄ has a formula selected from the group consisting of (G₂S)ₙ, (GS)ₙ, (GSGGS). (SEQ ID NO: 606), (GGGS)ₙ (SEQ ID NO: 607),(GGGGS)ₙ (SEQ ID NO: 608), and (GSSGGS)ₙ (SEQ ID NO: 609), wherein n is an integer of at least 1.

Embodiment 475 comprises the method of any one of embodiments 460-474, wherein the tumor specific protease is selected from the group consisting of metalloprotease, serine protease, cysteine protease, threonine protease, and aspartic protease.

Embodiment 476 comprises the method of any one of embodiments 460474, wherein L₃ or L₄ comprises a urokinase cleavable amino acid sequence, a matriptase cleavable amino acid sequence, or a matrix metalloprotease cleavable amino acid sequence.

Embodiment 477 comprises the method of any one of embodiments 460474, wherein L₃ or L₄ comprises a sequence according to any one of SEQ ID NOs: 21-22 and 107-136.

Embodiment 478 comprises the method of embodiments 461 or 462, wherein L₃ is bound to N-terminus of D.

Embodiment 479 comprises the method of embodiments 461 or 462, wherein L₃ is bound to C-terminus of D.

Embodiment 480 comprises the method of embodiments 461 or 462, wherein L₄ is bound to N-terminus of E.

Embodiment 481 comprises the method of embodiments 461 or 462, wherein La is bound to C-terminus of E.

Embodiment 482 comprises the method of any one of embodiments 460 and 462-479, wherein P₃ becomes unbound from D when L₃ is cleaved by the tumor specific protease thereby exposing D to CD3.

Embodiment 483 comprises the method of any one of embodiments 460479, wherein P₄ becomes unbound from E when L₄ is cleaved by the tumor specific protease thereby exposing E to the tumor antigen.

Embodiment 484 comprises the method of any one of embodiments 439, 441-458, and 461, wherein P₃ impairs binding of D to CD3.

Embodiment 485 comprises the method of any one of embodiments 460, 462479, 482, and 484, wherein P₃ is bound to D through ionic interactions, electrostatic interactions, hydrophobic interactions, Pi-stacking interactions, and H-bonding interactions, or a combination thereof.

Embodiment 486 comprises the method of any one of embodiments 460, 462-479, 482, and 484-485, wherein P₃ is bound to D at or near an antigen binding site.

Embodiment 487 comprises the method of any one of embodiments 460, 462-479, 482, and 485-486, wherein P₃ becomes unbound from D when L₃ is cleaved by the tumor specific protease thereby exposing D to CD3.

Embodiment 488 comprises the method of any one of embodiments 460, 462479, 482, and 485-487, wherein P₃ has less than 70% sequence identity to CD3.

Embodiment 489 comprises the method of any one of embodiments 460, 462-479, 482, and 485-487, wherein P₃ has less than 70% sequence identity to CD3.

Embodiment 490 comprises the method of any one of embodiments 460, 462-479, 482, and 485-487, wherein P₃has less than 85% sequence identity to CD3.

Embodiment 491 comprises the method of any one of embodiments 460, 462-479, 482, and 484-486, wherein P₃ has less than 90% sequence identity to CD3.

Embodiment 492 comprises the method of any one of embodiments 460, 462479, 482, and 484-486, wherein P₃ has less than 95% sequence identity to CD3.

Embodiment 493 comprises the method of any one of embodiments 460, 462-479, 482, and 484-486, wherein P₃ has less than 98% sequence identity to CD3.

Embodiment 494 comprises the method of any one of embodiments 460, 462-479, 482, and 484-486, wherein P₃ has less than 99% sequence identity to CD3.

Embodiment 495 comprises the method of any one of embodiments 460, 462-479, 482, and 484-486, wherein P₃ comprises the amino acid sequence according to SEQ ID NO: 625 or SEQ ID NO: 626.

Embodiment 496 comprises the method of any one of embodiments 460, 462479, 482, and 484-495, wherein P₃ comprises a de novo amino acid sequence that shares less than 10% sequence identity to CD3.

Embodiment 497 comprises the method of any one of embodiments 461-496, wherein P₄ impairs binding of E to the tumor antigen.

Embodiment 498 comprises the method of any one of embodiments 461-497. wherein P₄ is bound to E through ionic interactions, electrostatic interactions, hydrophobic interactions, Pi-stacking interactions, and H-bonding interactions, or a combination thereof.

Embodiment 499 comprises the method of any one of embodiments 461-498, wherein P₄ is bound to E at or near an antigen binding site.

Embodiment 500 comprises the method of any one of embodiments 461-499, wherein P₄ becomes unbound from E when L. is cleaved by the tumor specific protease thereby exposing E to the tumor antigen.

Embodiment 501 comprises the method of any one of embodiments 461-500 wherein P₄ has less than 70% sequence identity to the tumor antigen.

Embodiment 502 comprises the method of any one of embodiments 461-500, wherein P₄ has less than 80% sequence identity to the tumor antigen.

Embodiment 503 comprises the method of any one of embodiments 461-500, wherein P₄ has less than 85% sequence identity to the tumor antigen.

Embodiment 504 comprises the method of any one of embodiments 461-500 wherein P₄ has less than 90% sequence identity to the tumor antigen.

Embodiment 505 comprises the method of any one of embodiments 461-500, wherein P₄ has less than 95% sequence identity to the tumor antigen.

Embodiment 506 comprises the method of any one of embodiment 461-505, wherein P₄ comprises a de novo amino acid sequence that shares less than 10% sequence identity to the tumor antigen.

Embodiment 507 comprises the method of any one of embodiments 460-500, wherein P₃ or P₄ comprises a peptide sequence of at least 5 amino acids in length.

Embodiment 508 comprises the method of any one of embodiments 460-500, wherein P₃ or P₄ comprises a peptide sequence of at least 6 amino acids in length.

Embodiment 509 comprises the method of any one of embodiments 460-500, wherein P₃ or P₄ comprises a peptide sequence of at least 10 amino acids in length.

Embodiment 510 comprises the method ofany one of embodiments 460-500, wherein P₃ or P comprises a peptide sequence of at least 10 amino acids in length and no more than 20 amino acids in length.

Embodiment 511 comprises the method of any one of embodiments 460-500, wherein P₃ or P₄ comprises a peptide sequence of at least 16 amino acids in length.

Embodiment 512 comprises the method of any one of embodiments 460-500, wherein P₃ or P₄ comprises a peptide sequence of no more than 40 amino acids in length.

Embodiment 513 comprises the method of any one of embodiments 460-500, wherein P₃ or P₄ comprises at least two cysteine amino acid residues.

Embodiment 514 comprises the method of any one of embodiments 460-513, wherein P₃ or P comprises a cyclic peptide or a linear peptide.

Embodiment 515 comprises the method of any one of embodiments 460-514, wherein P₃ or P₄ comprises a cyclic peptide.

Embodiment 516 comprises the method of any one of embodiments 460-514. wherein P₃ or P₄ comprises a linear peptide.

Embodiment 517 comprises the method of any one of embodiments 460-500, wherein P₄ comprises the amino acid sequence according to SEQ ID NO: 645.

Embodiment 518 comprises the method of embodiment 436, wherein the tumor antigen comprises EGFR, and the T cell engager comprises the amino acid sequences of SEQ ID NOs: 643 and 644.

Embodiment 519 comprises the method of embodiment any one of embodiments 461-499, wherein P₄ comprises the amino acid sequence according to any one of SEQ ID NOs: 693-695.

Embodiment 520 comprises the method of embodiment 436, wherein the tumor antigen comprises TROP2, and the T cell engager comprises the amino acid sequences of SEQ ID NOs: 679 and 680, SEQ ID NOs: 681 and 682, SEQ ID NOs: 683 and 684, SEQ ID NOs: 685 and 686, SEQ ID NOs: 687 and 688. SEQ ID NOs: 689 and 690, and SEQ ID NOs: 691 and 692.

Embodiment 521 comprises the method of embodiment 436, wherein the tumor antigen comprises TROP2, and the T cell engager comprises the amino acid sequences of SEQ ID NOs: 4452 and 4453, SEQ ID NOs: 4454 and 4455, SEQ ID NOs: 4456 and 4457, SEQ ID NOs: 4458 and 4459, SEQ ID NOs: 4460 and 4461, SEQ ID NOs: 4462 and 4463, or SEQ ID NOs: 4464 and 4465.

Embodiment 522 comprises the method of embodiment 436, wherein the tumor antigen comprises PSMA, and the T cell engager comprises the amino acid sequences of SEQ ID NOs: 657 and 658.

Embodiment 523 comprises a vector comprising the isolated recombinant nucleic acid molecule of embodiment 424.

### EXAMPLES

Example 1: Tumor or Cell Killing with Polypeptide Complexes that Bind PSMA and CD28 and T Cell Engagers (TCEs)

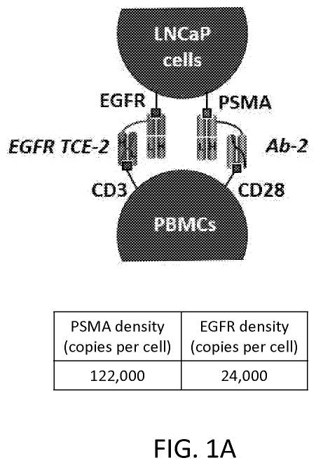

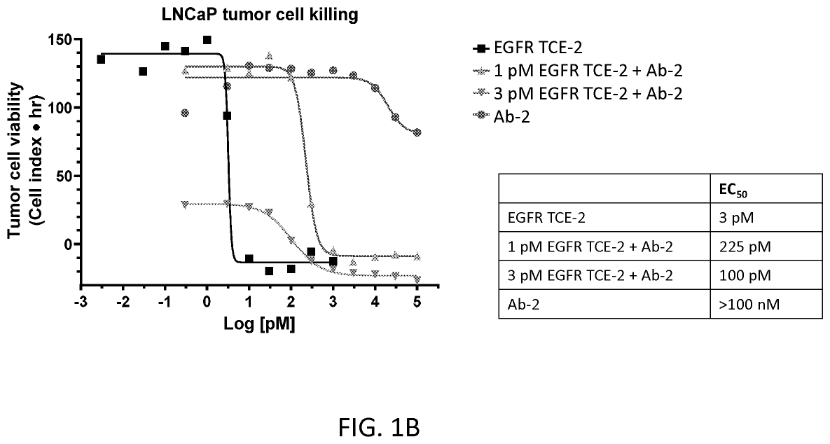

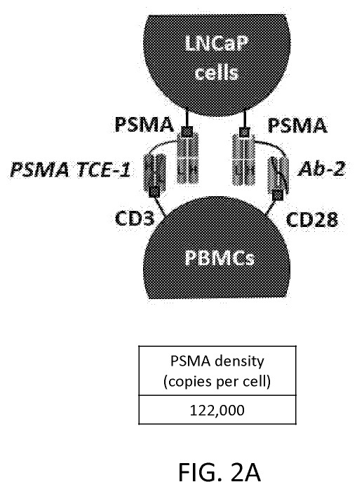

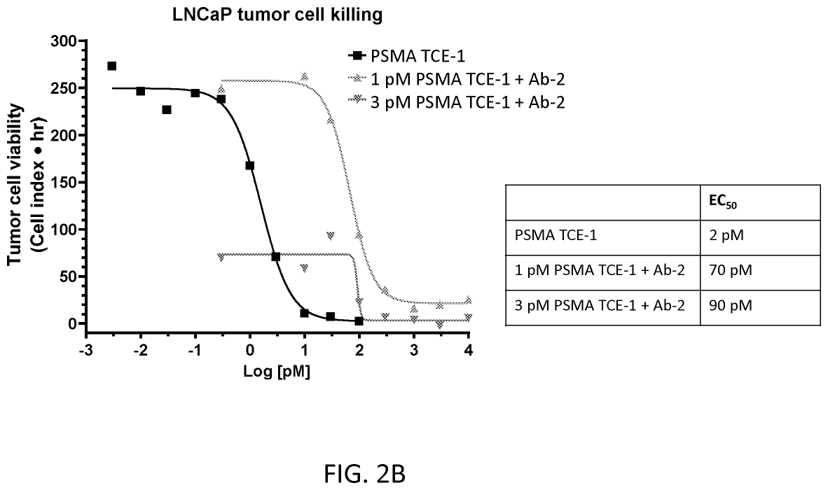

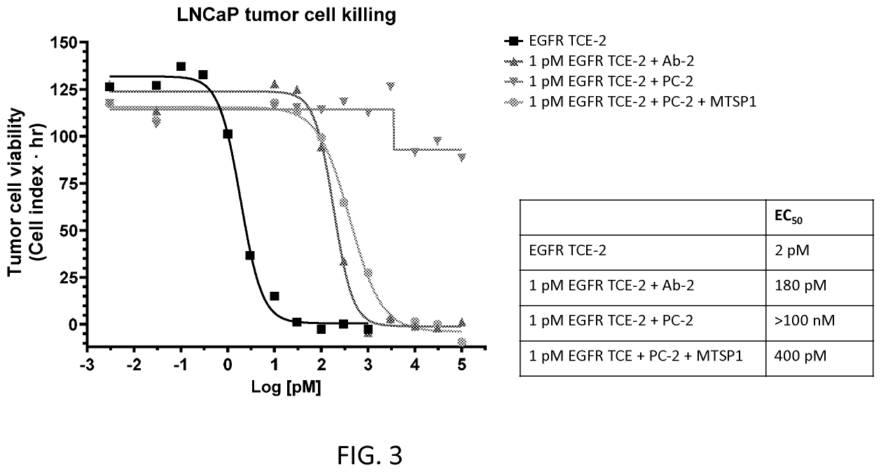

Costimulatory activity from CD28 targeted agents requires TCR signaling (signal 1) driven by TCR-MHC interactions or CD3 targeted TCEs. In the absence of signal 1, CD28 costimulators are inactive. CD3 targeted TCEs create signal I through TCE signaling without the need for TCR/MHC interactions. Accordingly, the multispecific antibodies of the present disclosure that bind PSMA and CD28 were evaluated for tumor cell killing in combination with TCEs that bind EGFR and CD3 (FIG. 1A) or PSMA and CD3 (FIG. 2A). Tumor cell killing was evaluated using an in vitro MABEL assay utilizing human peripheral blood mononuclear cell (hPBMC) co-cultured with the PSMA positive tumor cell line, LNCaP. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight on a 96 well E-Plate. The following day, polypeptide complexes titrated in human serum supplemented medium along with 50,000 human PBMCs were added to the wells. Cell index measurements were taken every 10 minutes for an additional 96 hours. The cell index times number of hours (tumor cell growth kinetics) was then plotted versus concentration of polypeptide complex where the concentration required to reduce the tumor growth 50% (EC₅₀) was calculated using Graphpad Prism software. FIG. 1B shows tumor cell killing with EGFR TCE-2 alone, Ab-2 alone, and a combination of Ab-2 with different concentrations of EGFR TCE-2. FIG. 2B demonstrates tumor cell killing with PSMA TCE-1 alone and a combination of Ab-2 with different concentrations of PSMA TCE-1. FIG. 3 shows tumor cell killing by EGFR TCE-2 alone, EGFR TCE-2 combined with Ab-2, and EGFR TCE-2 combined with PC-2 which binds PSMA and CD28 and includes peptide-41 as a mask for CD28. As shown in FIG. 3, PC-2 regains activity similar to non-masked Ab-2 with cleavage of the peptide mask by MTSP1.

### Example 2: Cytokine Production with Polypeptide Complexes that Bind PSMA and CD28 and TROP2 TCEs

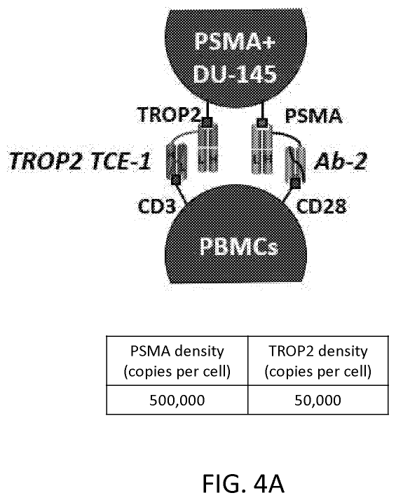

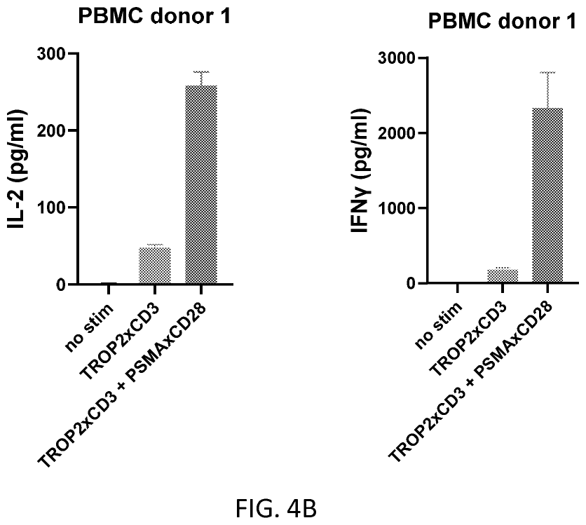

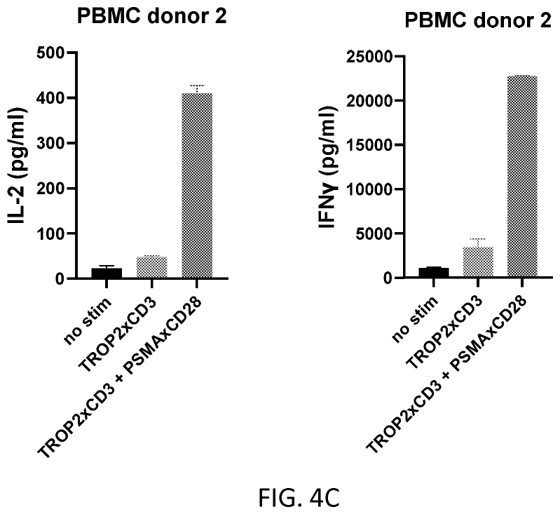

Multispecific antibodies of the present disclosure that bind PSMA and CD28 were evaluated for their ability to enhance TROP2 TCE induced production of interleukin 2 (IL-2) and interferon gamma (IFNγ). Specifically, polypeptide complexes were evaluated in a functional in vitro T cell activation assay using the recombinant PSMA expressing tumor cell line, DU-145. T cell activation was measured via cytokine release. 10,000 tumor cells were added per well and allowed to adhere overnight in a 96 well plate. The following day polypeptide complexes titrated in human serum supplemented medium along with 50,000 human PBMCs were added to the wells. Supernatants were harvested 72 hrs later. Cytokine levels were measured from the harvested supernatants using a Meso Scale Discovery kit following manufacturer's instructions. FIG. 4B illustrates IL-2 and IFNγ cytokine production with no stimulation (“no stim”), stimulation with TROP2 TCE-1 (“TROP2xCD3”), and stimulation with a combination of TROP2 TCE-1 and Ab-2 (“TROP2xCD3+PSMAxCD28”) with PBMC donor 1. FIG. 4C illustrates IL-2 and IFNγ cytokine production with no stimulation (“no stim”), stimulation with TROP2 TCE-1 (“TROP2xCD3”), and stimulation with a combination of TROP2 TCE-1 and Ab-2 (“TROP2xCD3+PSMAxCD28”) with PBMC donor 2. As can be seen, Ab-2 enhances TROP2 TCE induced production of cytokines IL-2 and IFNγ.

### Example 3: Costimulatory Activity of Polypeptide Complexes that Bind PSMA and CD28 in a Tumor Cell/PBMC Mixed Lymphocyte Reaction (MLR) Co-Culture System

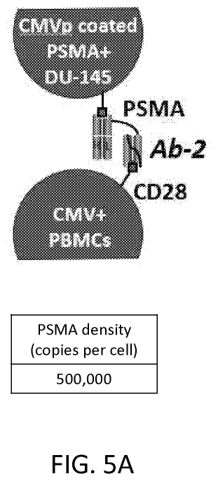

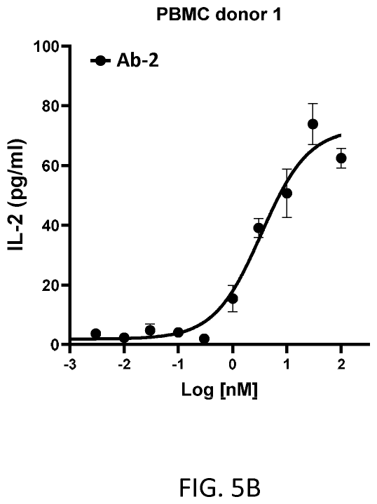

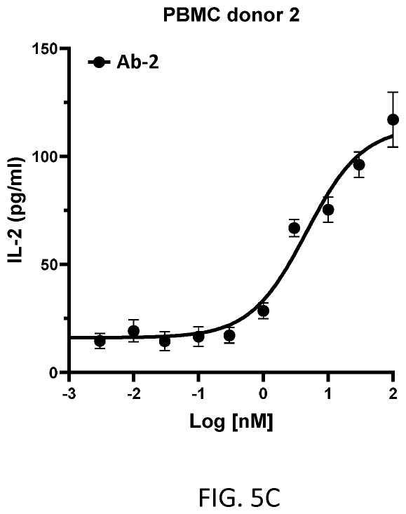

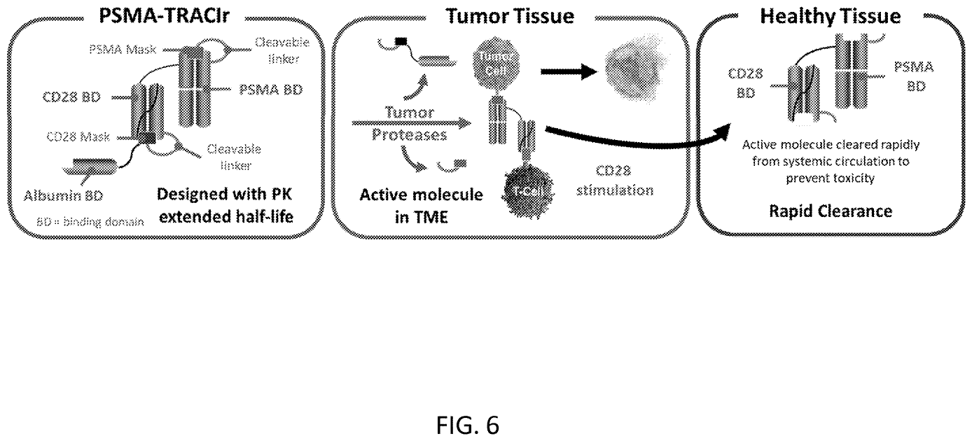

The costimulatory activity of polypeptide complexes of the present disclosure was assessed using a cell-based assay wherein signal 1 is provided by cytomegalovirus peptide (CMVp) coated tumor cells and PBMCs from CMV positive donors that carry T cells with TCRs that recognize CMVp-MHC complexes (see FIG. 5A). To evaluate the costimulatory activity of the polypeptide complexes, a MLR system was established based on coculture of healthy human donor PBMCs and recombinant PSMA expressing tumor cell line, DU-145. To ensure presence of active antigen presentation, DU-145 cells were loaded with a CMV peptide pool. To ensure T cell recognition of this peptide, PBMCs derived from CMV positive donors were used in the MLR reaction. In brief, 50,000 DU-145 cells were seeded per well of 96-well plates in presence of 1 ug/mL of CMV peptide pool, and incubated overnight in a 37° C., 5% CO₂ humidified incubator. The following day, appropriate serial dilutions of the polypeptide complexes were prepared in a complete cell culture medium and added to wells containing peptide-coated DU-145 tumor cells. Healthy donor PBMCs (150,000 cells) were resuspended in cell culture medium and added in appropriate wells. PBMCs were incubated with the polypeptide complexes and DU-145 cells for 72 h in a 37° C., 5% CO₂ humidified incubator. Cell culture supematants were harvested and stored at −20° C. until cytokine analysis. Soluble IL-2 was measured in cell culture supematants using Meso scale discovery kit following manufacturer's instructions. IL-2 induction was plotted against logarithmic concentration of the polypeptide complexes. As shown in FIGS. 5B-5C, Ab-2 enhances IL-2 production in the MLR system in a dose-dependent fashion.

### Example 4: Tumor Cell Killing with Polypeptide Complexes (PCs) that Bind PSMA and CD28 and TCEs

Polypeptide complexes of the present disclosure that bind PSMA and CD28 were evaluated for tumor cell killing in combination with TCEs that bind EGFR and CD3 (EGFR TCEs) or TCEs that bind PSMA and CD3 (PSMA TCEs). Polypeptide complexes were evaluated in a functional in vitro tumor cell killing assay using the PSMA positive tumor cell lines, LNCaP, 22Rv1, or VCaP. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight on a 96 well E-Plate. The following day polypeptide complexes titrated in human serum supplemented medium along with 30,000 human PBMCs or pan T cells were added to the wells. Cell index measurements were taken every 10 minutes for an additional 120 hours. The cell index times number of hours (tumor cell growth kinetics) was then plotted versus concentration of polypeptide complex where the concentration required to reduce the tumor growth 50% (1C50) was calculated using Graphpad Prism software.

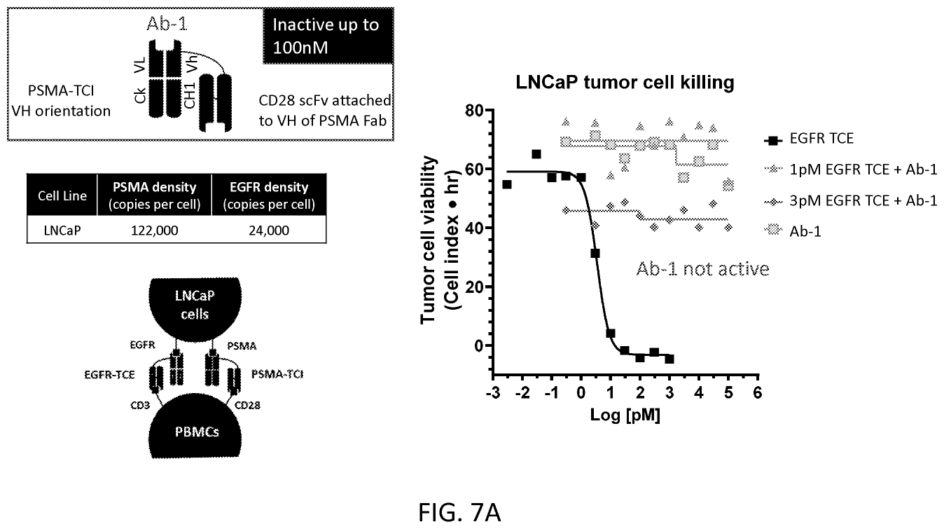

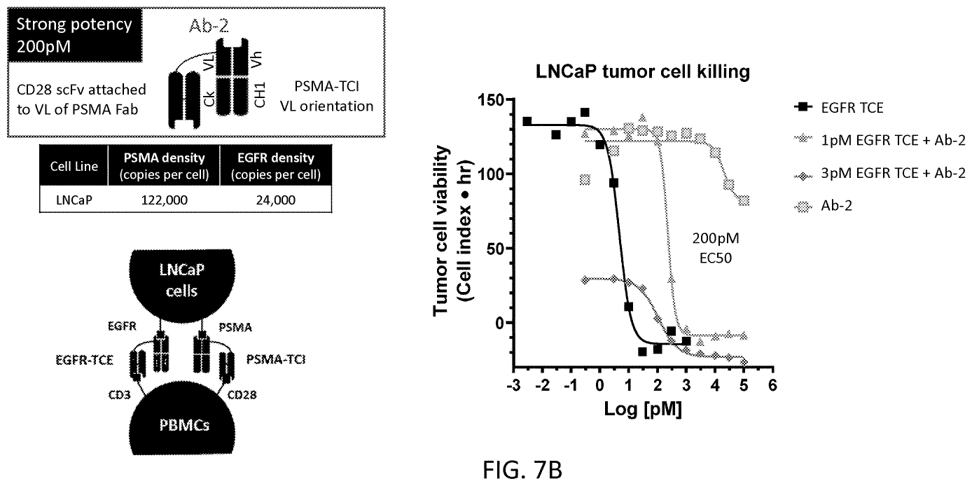

FIG. 7A shows tumor cell killing of LNCaP tumor cells by EGFR TCE-1 alone, different concentrations of EGFR TCE-1 in combination with Ab-1, and Ab-1 alone. FIG. 7B shows tumor cell killing of LNCaP tumor cells by EGFR TCE-1 alone, different concentrations of EGFR TCE−1 in combination with Ab-2, and Ab-2 alone. In Ab-1 the CD28 binding domain is attached to the variable heavy chain (Vh) of the PSMA binding domain, whereas in Ab-2 the CD28 binding domain is connected to the variable light chain (VI) of the PSMA binding domain. As can be seen, Ab-2 exhibits strong tumor cell killing potency in combination with EGFR TCE-1 with an EC₅oof 200 pM, whereas Ab−1 is inactive up to 100 nM. The data indicates higher activity when the CD28 binding domain is connected to the VI of the PSMA binding domain.

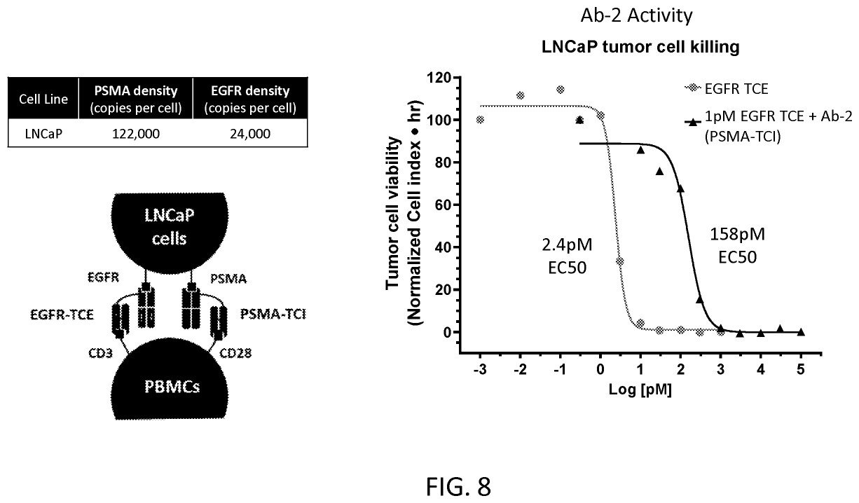

FIG. 8 shows tumor cell killing of LNCaP tumor cells by EGFR TCE-1 alone and EGFR TCE-1 in combination with Ab-2 along with EC₅₀ₛ, demonstrating that Ab-2 exhibits costimulatory activity in the presence of the EGFR TCE-1 signal 1.

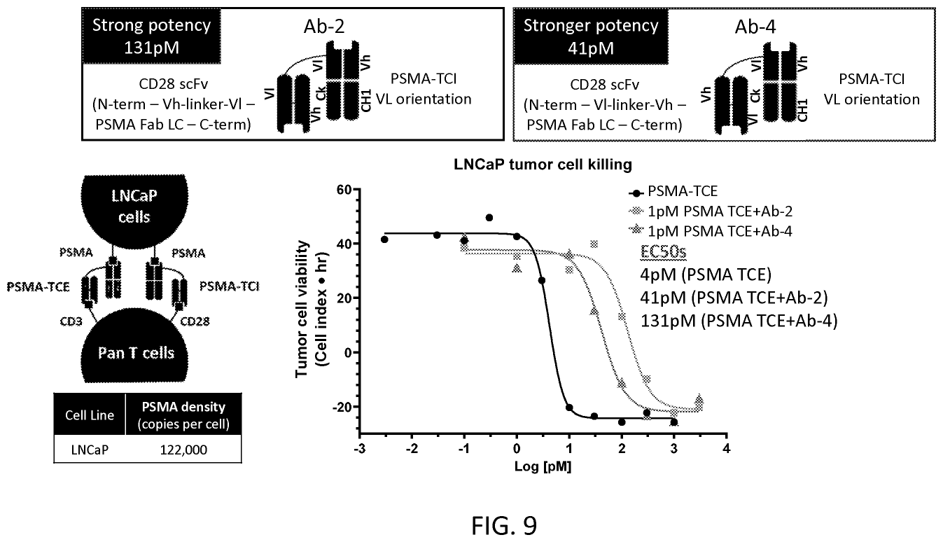

FIG. 9 shows tumor cell killing of LNCaP tumor cells by PSMA TCE-1 alone and in combination with Ab-2 or Ab-4. Ab-2 has a CD28 scFv binding domain with a Vh-linker-Vl geometry, and Ab-4 has a CD28 scFv binding domain with an Vl-linker-Vh geometry. As can be seen Ab-4 showed a stronger tumor cell killing potency, indicating that the VI-linker-Vh geometry is the preferred configuration.

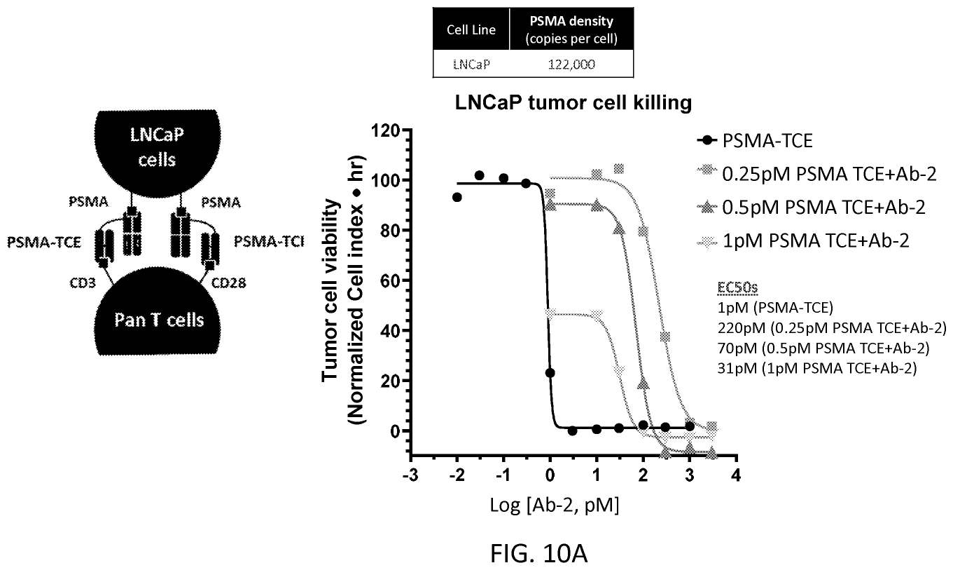

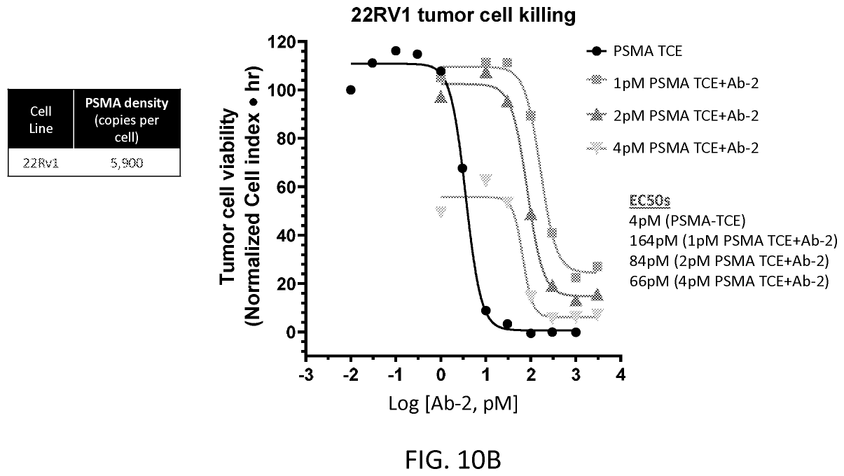

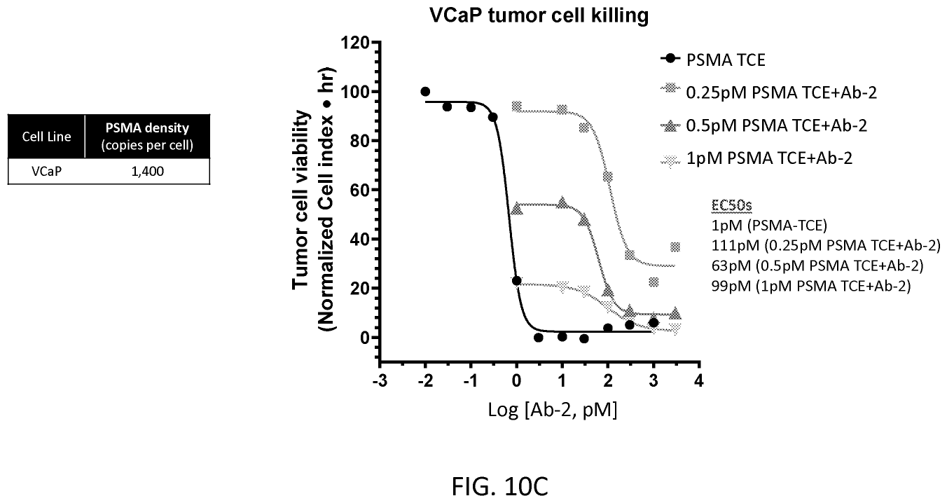

FIGS. 10A-10C show tumor cell killing of different cancer cell lines with different PSMA expression in the presence of PSMA TCE-1 or PSMA TCE-1 in the presence of Ab-2. Specifically, FIG. 10A shows killing of LNCaP tumor cells with a PSMA density of 122,000 copies per cell, FIG. 10B shows killing of 22Rv1 tumor cells with a PSMA density of 5,900 copies per cell, and FIG. 10C shows killing of VcaP tumor cells with a PSMA density of 1,400 copies per cell. As shown, Ab-2 maintains full cytolytic activity against prostate cancer lines with low PSMA expression.

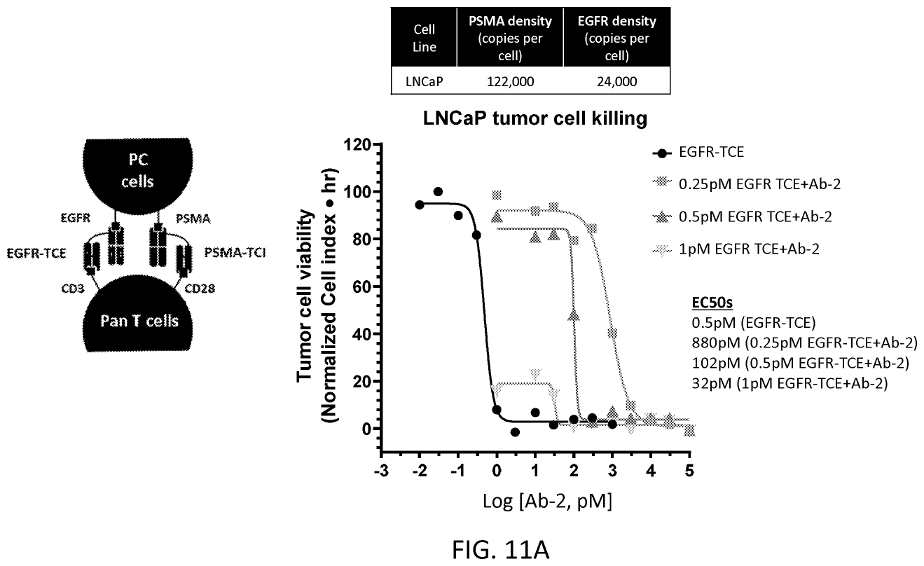

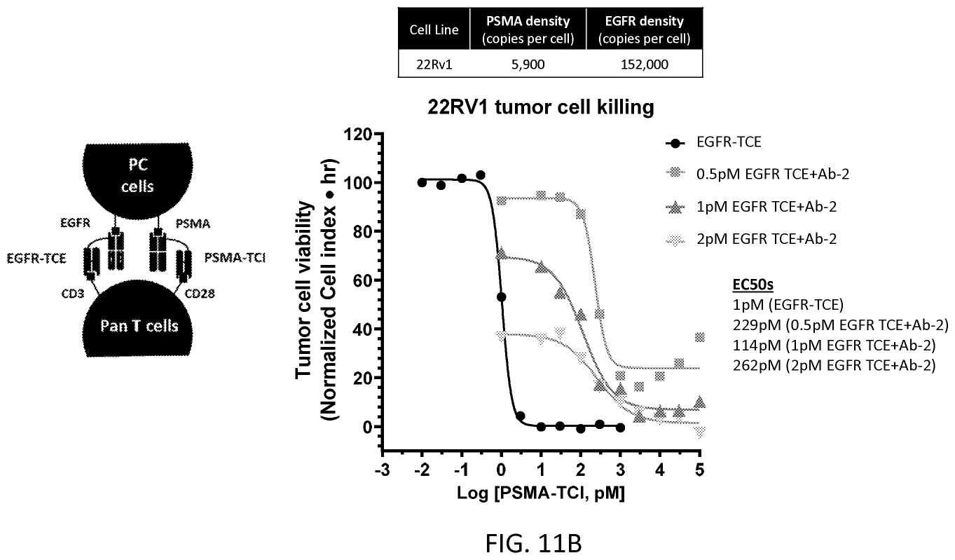

FIG. 11A-11B show tumor cell killing of different cancer cell lines with different PSMA and EGFR expression with EGFR TCE-1 alone or EGFR TCE-1 in the presence of Ab-2. Specifically, FIG. 11A shows killing of LNCaP tumor cells (PSMA expression density of 122,000 copies per cell; EGFR expression density of 24,000 copies per cell), and FIG. 11B shows killing of 22Rv1 tumor cells (PSMA expression density of 5,900 copies per cell; EGFR expression density of 152.000 copies per cell). As shown, Ab-2 maintains full cytolytic activity against prostate cell lines with low PSMA expression.

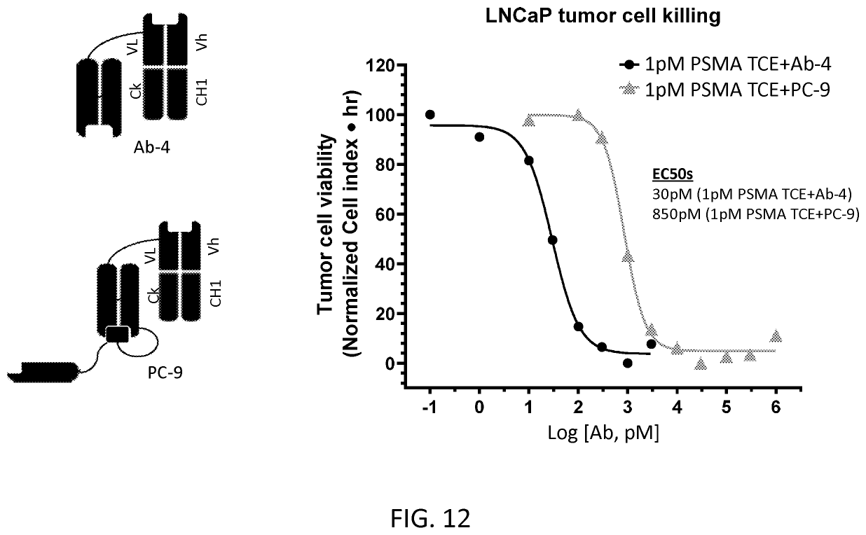

FIG. 12 shows tumor cell killing of LNCaP cancer cells with PSMA TCE-1 in combination with Ab-4 without peptide masks, and PSMA TCE-1 in combination with PC-9 having a single CD28 binding domain mask (peptide-41). As shown, masking of the CD28 binding domain in PC-9 reduces tumor cell killing potency by about 30-fold relative to non-masked Ab-4.

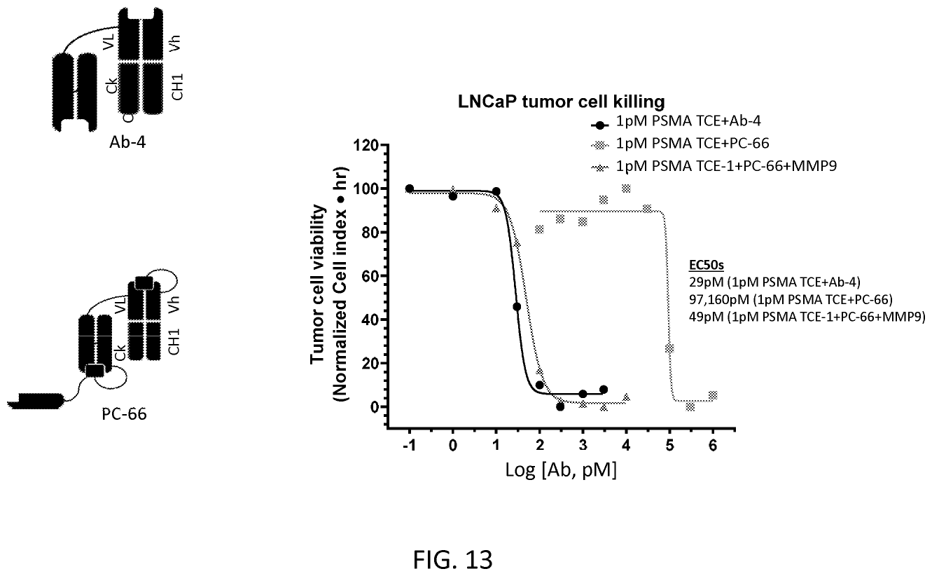

FIG. 13 shows tumor cell killing of LNCaP cancer cells with PSMA TCE-1 in combination with Ab-4 (no masks), PSMA TCE-1 in combination with double-masked PC-66 having a CD28 binding domain mask (peptide-41) and a PSMA binding domain mask (peptide-274), and PSMA TCE-1 in combination with PC-66 after MMP9 protease treatment (mask cleavage). As can be seen, double masking in PC-66 reduces tumor cell killing potency by more than 3,000-fold relative to the non-masked Ab-4 construct. Additionally. mask cleavage by treatment of PC-66 with matrix metalloproteinase 9 (MMP-9) restores the tumor cell killing potency to an EC₅₀ similar to that of non-masked Ab-4.

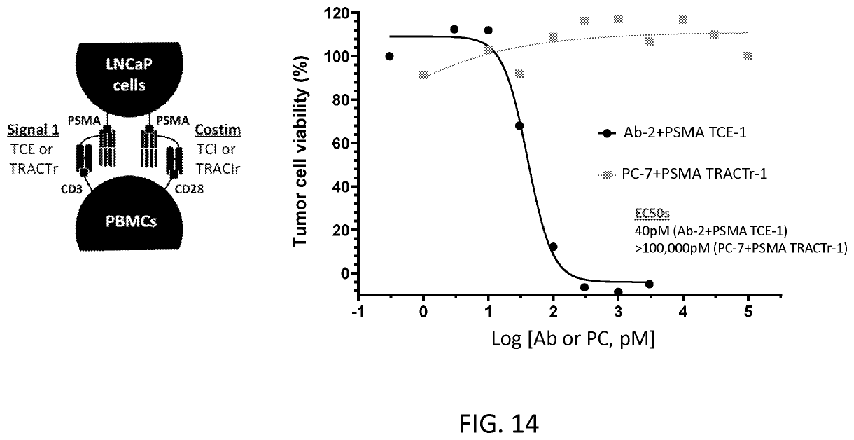

FIG. 14 shows tumor cell killing of LNCaP cancer cells over 4 days with a combination of non-masked Ab-2 and PSMA TCE-1, and with a combination of single-masked PC-7 having peptide-41 mask and masked PSMA TRACTr-1. As shown in the figure, T cell activation was shown to be peptide mask dependent as tumor cell killing was observed with the combination of non-masked Ab-2 and PSMA TCE-1 but not with the combination of masked PC-7 and masked PSMA TRACTr-1. Cytotoxicity of Double-Masked Polypeptides that Bind PSMA and CD28

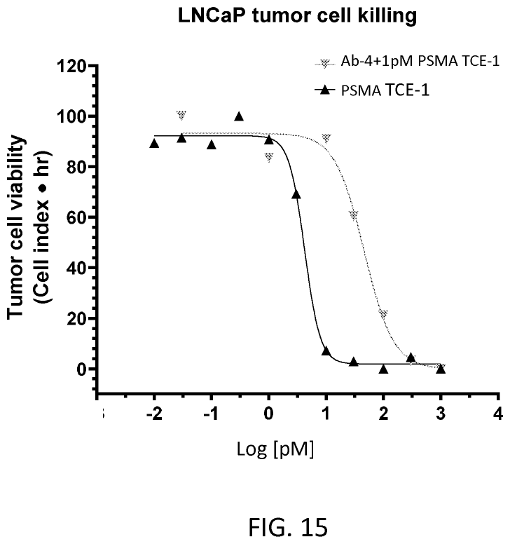

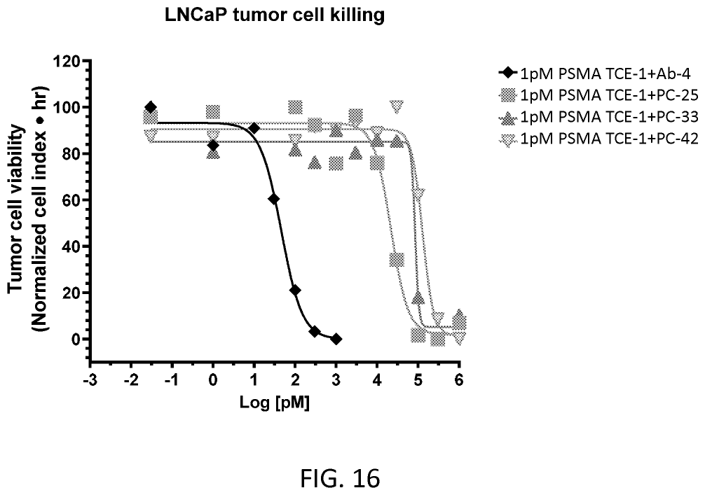

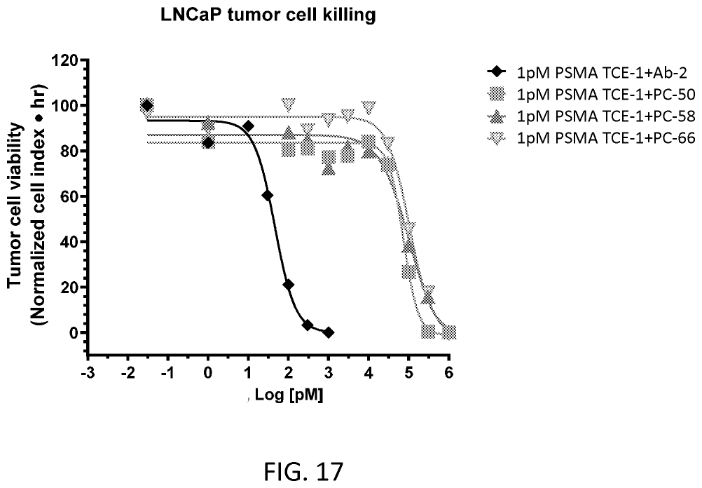

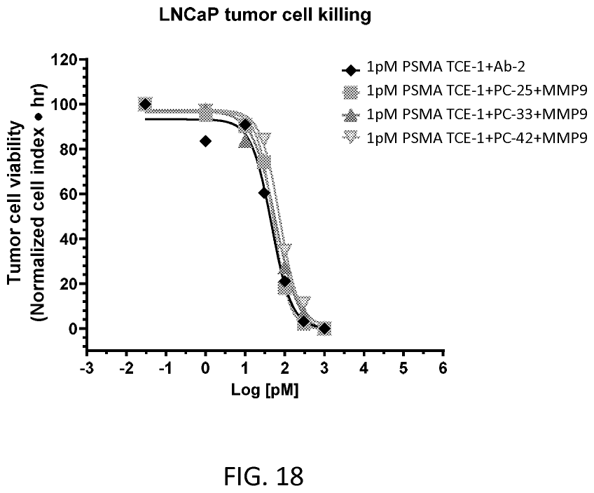

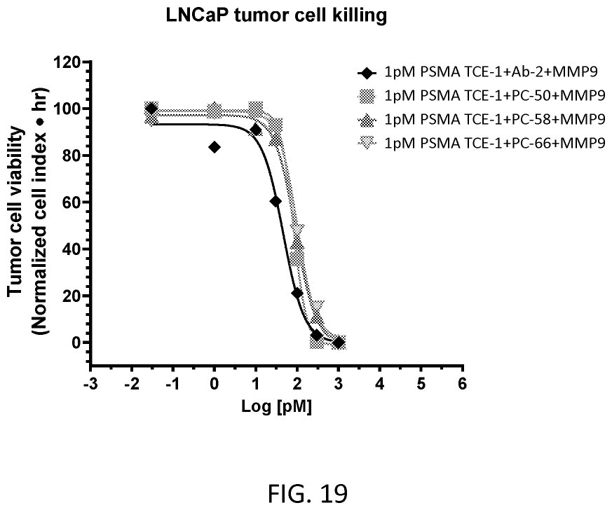

FIG. 15 shows tumor cell killing of LNCaP cells with PSMA TCE-1 alone and with a combination of PSMA TCE-1 and Ab-4. FIG. 16 shows tumor cell killing of LNCaP cells with PSMA TCE-1 and Ab-4, PSMA TCE-1 and PC-25, PSMA TCE-1 and PC-33, and PSMA TCE-1 and PC-42. FIG. 18 shows tumor cell killing of LNCaP cells with PSMA TCE-1 and Ab-4, PSMA TCE-1 and PC-25 with MMP9 protease treatment, PSMA TCE-1 and PC-33 with MMP9 protease treatment, and PSMA TCE-1 and PC-42 with MMP9 protease treatment. FIG. 17 shows tumor cell killing of LNCaP cancer cells with PSMA TCE-1 and Ab-2. PSMA TCE-1 and PC-50, PSMA TCE-1 and PC-58, and PSMA TCE-1 and PC-66. FIG. 19 shows tumor cell killing of LNCaP cancer cells with PSMA TCE-1 and Ab-4, PSMA TCE-1 and PC-50 with MMP9 protease treatment, PSMA TCE-1 and PC-58 with MMP9 protease treatment, and PSMA TCE-1 and PC-66 with MMP9 protease treatment. The experiments were carried out in the presence of 1 pM PSMA TCE-1 which provides signal 1. As can be seen from FIG. 18 and FIG. 19. cleavage of the peptide masks by protease treatment shifts the tumor cell killing potencies of the PCs to that of non-masked Ab-2. Corresponding EC₅₀ values for the plots of FIGS. 15-19 are shown in Table 18. Polypeptide complexes with PSMA masks Peptide-233 (PC-42), Peptide-271 (PC-58), and Peptide-274 (PC-66) had the greatest potency shift relative to the unmasked Ab-4.

TABLE 18
LNCaP Tumor Cell Killing, Cytolytic Potencies

| | | | EC₅₀ shift relative |
|---|---|---|---|
| Peptide | PSMA Mask | EC₅₀ (pM) | to unmasked (pM) |
| Ab-4 | — | 41 | — |
| PC-25 | Peptide-219 | 21618 | 527x |
| PC-33 | Peptide-230 | 84490 | 2060x |
| PC-42 | Peptide-233 | 127624 | 3112x |
| PC-50 | Peptide-268 | 73282 | 1787x |
| PC-58 | Peptide-271 | 101381 | 2472x |
| PC-66 | Peptide-274 | 101535 | 2476x |

### Example 5. Cytokine Induction of Masked PSMAxCD28 Multispecific Antibodies

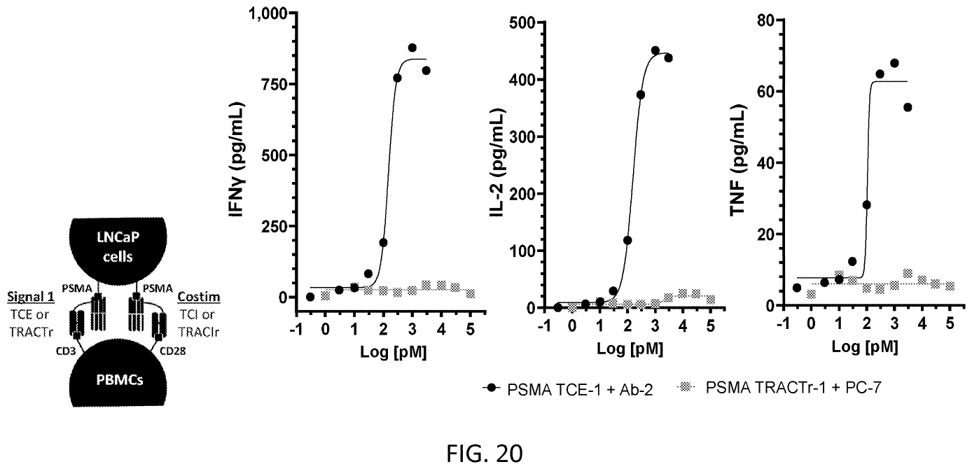

Multispecific antibodies of the present disclosure that bind PSMA and CD28 were evaluated for their ability to induce production of interleukin 2 (IL-2), interferon gamma (IFNγ), and tumor necrosis factor (TNF). Specifically, polypeptide complexes were evaluated in a functional in vitro T cell activation assay using the recombinant PSMA expressing tumor cell line, LNCaP, expressing 122,000 copies/cell. T cell activation was measured via cytokine release. 10,000 tumor cells were added per well and allowed to adhere overnight in a 96 well plate. The following day polypeptide complexes titrated in human serum supplemented medium along with 50,000 human PBMCs were added to the wells. Supematants were harvested 72 hrs later. Cytokine levels were measured from the harvested supernatants using a Meso Scale Discovery kit following manufacturer's instructions. FIG. 20 illustrates IL-2, IFNγ, and TNF cytokine production with PSMA TCE+Ab-2 and PSMA-TRACTr-1+PC-7. As can be seen, masking of the polypeptide complex prevents cytokine induction.

### Example 6. Binding of PSMA and CD28 by Polypeptide Complexes

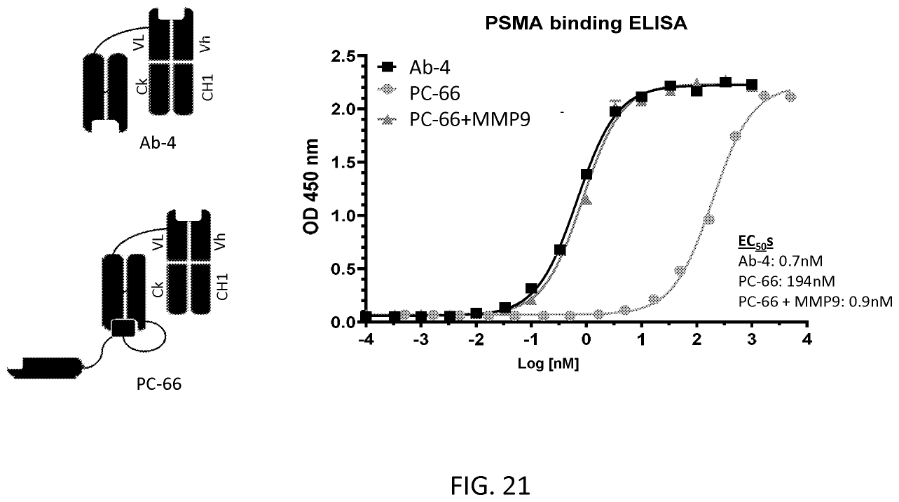

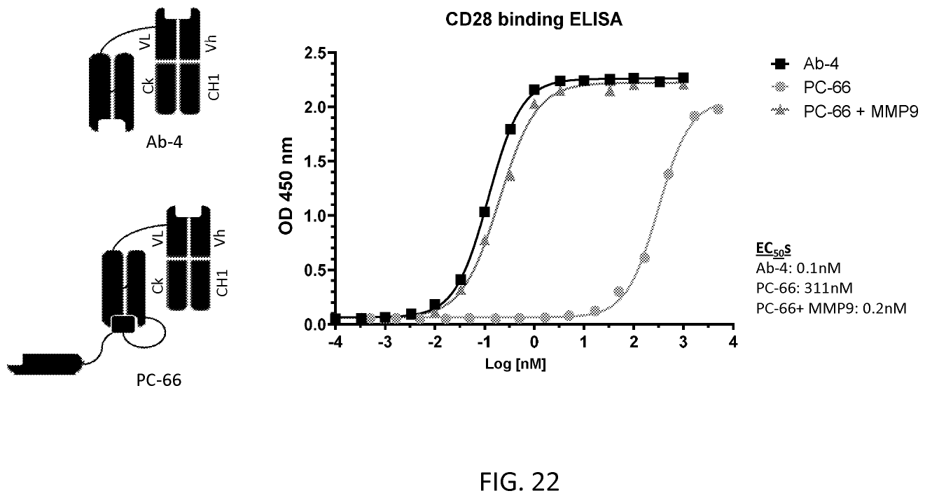

The polypeptide complex molecules of Table 13 were evaluated for their ability to bind PSMA as well as CD28 in a standard enzyme linked immunosorbent assay (ELISA) format. Binding of PSMA and CD28 was measured for polypeptide complexes Ab-4, and PC-66 in the presence or absence of matrix metalloproteinase 9 (MMP9). Briefly, biotinylated antigen was captured on neutravidin coated plates. Polypeptide complex molecules diluted in buffer were then added to the antigen coated plates. Bound polypeptide complex was detected using a standard horse radish peroxidase conjugate secondary antibody. The concentration of polypeptide complex required to achieve 50% maximal signal (EC50) was calculated in Graphpad Prism. FIGS. 21-22 illustrate polypeptide complex binding to PSMA and CD28. As can be seen, masking of the polypeptide complex inhibits target engagement. Target engagement is restored with cleavage of the masking peptide.

### Example 7: Anti-CD28 scFV Binding to Peptide Masks Equilibrium Binding via ELMS

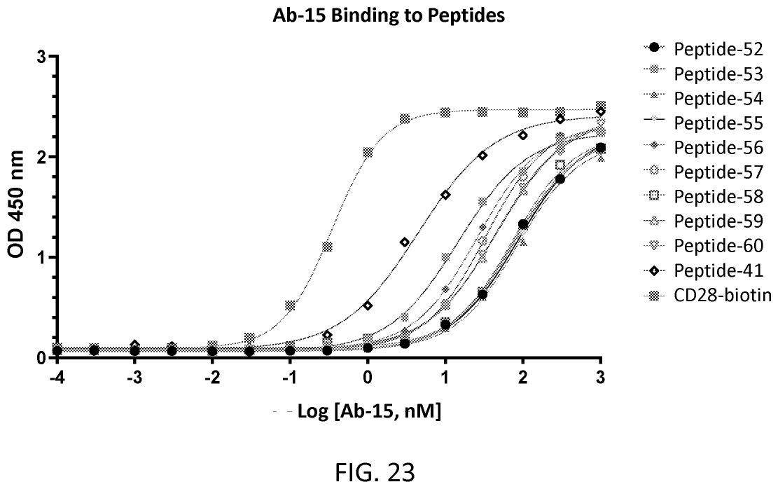

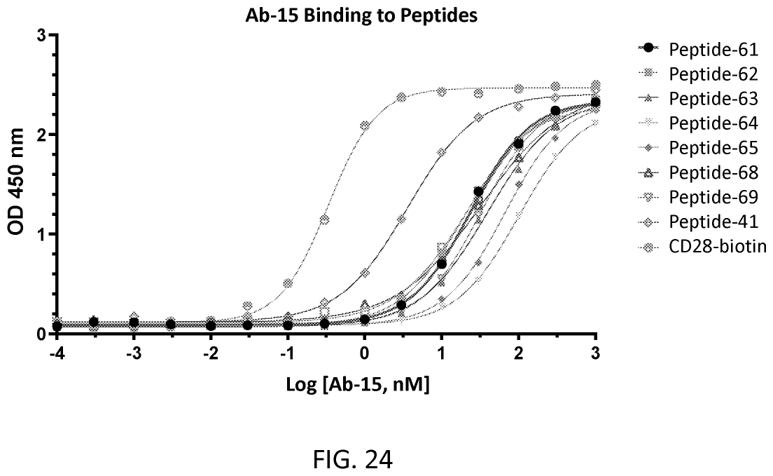

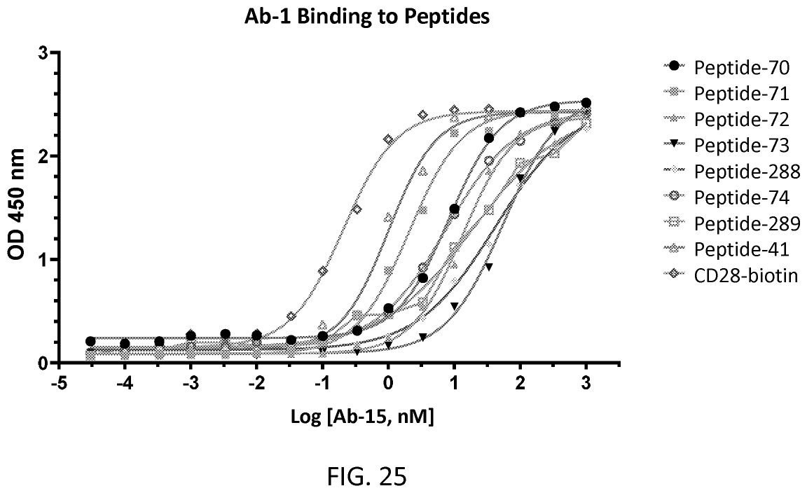

Peptides of Table 5 were evaluated for their ability to bind to human anti-CD28 scFv in a standard enzyme linked immunosorbent assay (ELISA) format. Specifically, peptides were evaluated for their ability to bind polypeptide sequence Ab-15 which is a bispecific antibody comprising the anti-CD28 scFv sequence of SEQ ID NO: 9 connected to a second antibody Fab by a GGGGS linker (see Table 11). Briefly, biotinylated peptides or CD28-biotin were captured on neutravidin coated plates. Ab-15 diluted in buffer was then added to the peptide captured plates. Bound antibody was detected using a standard horse radish peroxidase conjugate secondary antibody. The concentration of Ab-15 required to achieve 50% maximal signal (EC₅₀) was calculated using Graphpad Prism software. Binding curves for Ab-15 binding to peptides are shown in FIGS. 23-25, and the corresponding ECs for Ab-TS binding to the peptides are shown in Tables 20-21.

TABLE 19
Exemplary antibody sequences with a CD28 binding domain and
a second target binding domain.

| | | SEQ |
|---|---|---|
| Construct | Amino Acid Sequence | ID |
| Description | (N to C) | NO: |
| Ab-15 HC | QVQLVQSGAEVKKPGASVKVSCKASGYTFTSYYIHWVRQAPGQGL | 4466 |
| | EWIGSIYPGNVNTNYNEKFKDRATLTVDTSISTAYMELSRLRSDDT | |
| | AVYFCTRSHYGLDWNFDVWGQGTTVTVSSGGGGSGGGGSGGGGSDI | |
| | QMTQSPSSLSASVGDRVTITCHASQNIYVWLNWYQQKPGKAPKLLI | |
| | YKASNLHTGVPSRFSGSGSGTDFTLTISSLQPEDFATYYCQQGQTYP | |
| | YTFGGGTKVEIKGGGGSQVQLVQSGAEVKKPGSSVKVSCKTSGDTFS | |
| | TYAISWVRQAPGQGLEWMGGIIPIFGKAHYAQKFQGRVTITADESTS | |
| | TAYMELSSLRSEDTAVYFCARKFHFVSGSPFGMDVWGQGTTVTVSS | |
| | ASTKGPSVFPLAPSSKSTSGGTAALGCLVKDYFPEPVTVSWNSGALT | |
| | SGVHTFPAVLQSSGLYSLSSVVTVPSSSLGTQTYICNVNHKPSNTKVD | |
| | KKVEPKSC | |
| Ab-15 LC | EIVLTQSPATLSLSPGERATLSCRASQSVSSYLAWYQQKPGQAPRLLI | 4467 |
| | YDASNRATGIPARFSGSGSGTDFTLTISSLEPEDFAVYYCQQRSNWPT | |
| | FGQGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| Ab-16 LC | EIVLTQSPATLSLSPGERATLSCRASQSVSSYLAWYQQKPGQAPRLLI | 4468 |
| G44C, G235C | YDASNRATGIPARFSGSGSGTDFTLTISSLEPEDFAVYYCQQRSNWPT | |
| | FGQGTKVEIKRTVAAPSVFIFPPSDEQLKSGTASVVCLLNNFYPREAK | |
| | VQWKVDNALQSGNSQESVTEQDSKDSTYSLSSTLTLSKADYEKHKV | |
| | YACEVTHQGLSSPVTKSFNRGEC | |
| Ab-16 HC | QVQLVQSGAEVKKPGASVKVSCKASGYTFTSYYIHWVRQAPGQCLE | 4469 |
| G44C, | WIGSIYPGNVNTNYNEKFKDRATLTVDTSISTAYMELSRLRSDDTAV | |
| G235C | YFCTRSHYGLDWNFDVWGQGTTVTVSSGGGGSGGGGSGGGGSDIQ | |
| | MTQSPSSLSASVGDRVTITCHASQNIYVWLNWYQQKPGKAPKLLIY | |
| | KASNLHTGVPSRFSGSGSGTDFTLTISSLOPEDFATYYCQQGQTYPYT | |
| | FGCGTKVEIKGGGGSQVQLVQSGAEVKKPGSSVKVSCKTSGDTFST | |
| | YAISWVRQAPGQGLEWMGGIIPIFGKAHYAQKFQGRVTITADESTST | |
| | AYMELSSLRSEDTAVYFCARKFHFVSGSPFGMDVWGQGTTVTVSSA | |
| | STKGPSVFPLAPSSKSTSGGTAALGCLVKDYFPEPVTVSWNSGALTS | |
| | GVHTFPAVLQSSGLYSLSSVVTVPSSSLGTQTYICNVNHKPSNTKVD | |
| | KKVEPKSC | |

### Kinetic Binding via BLI

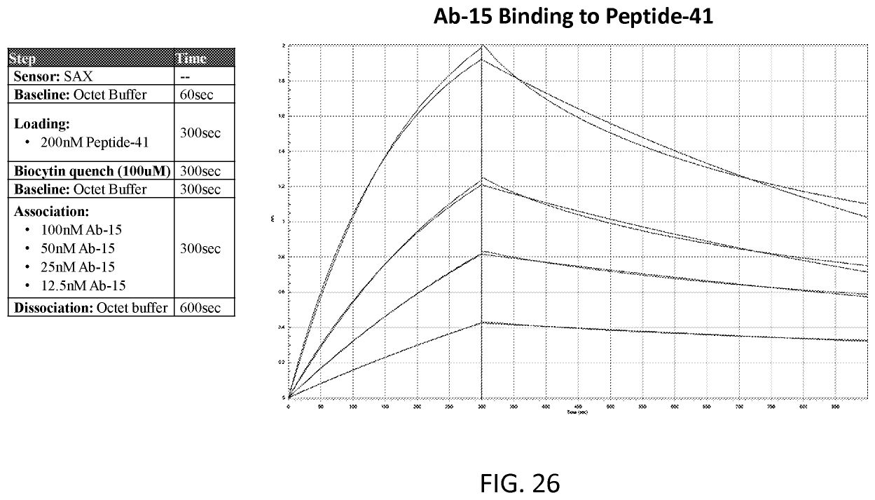

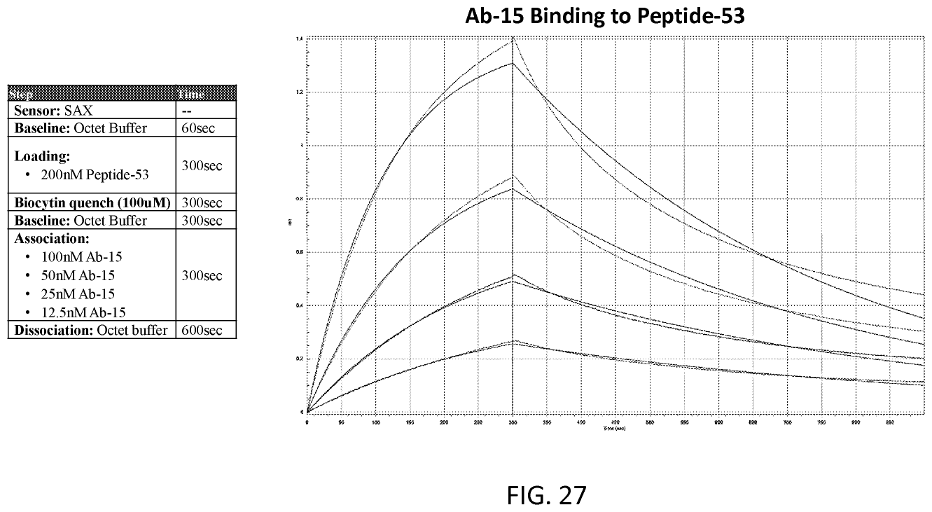

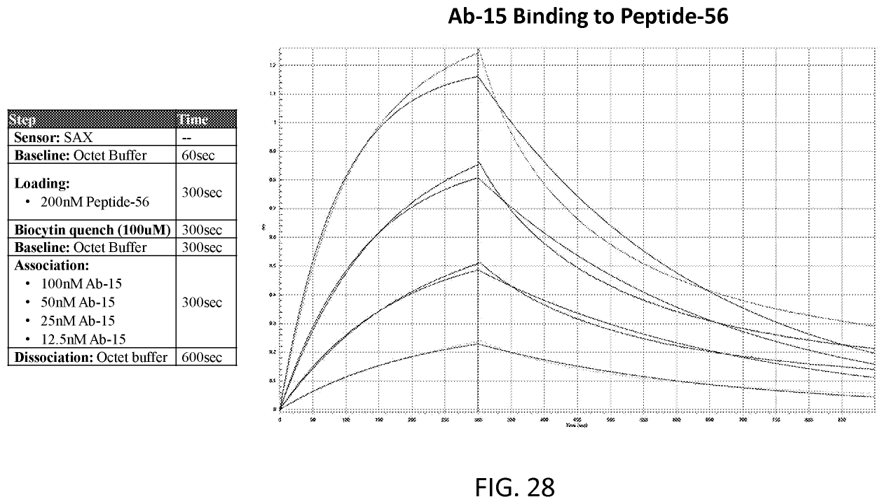

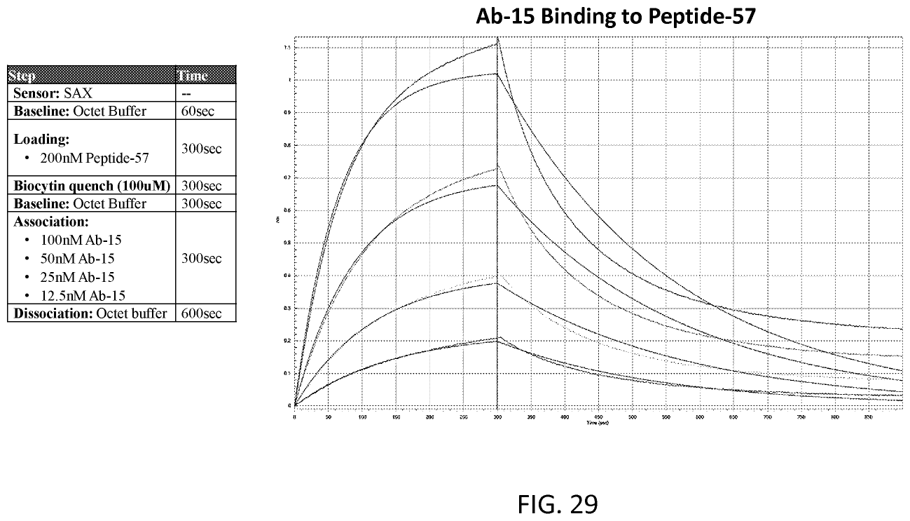

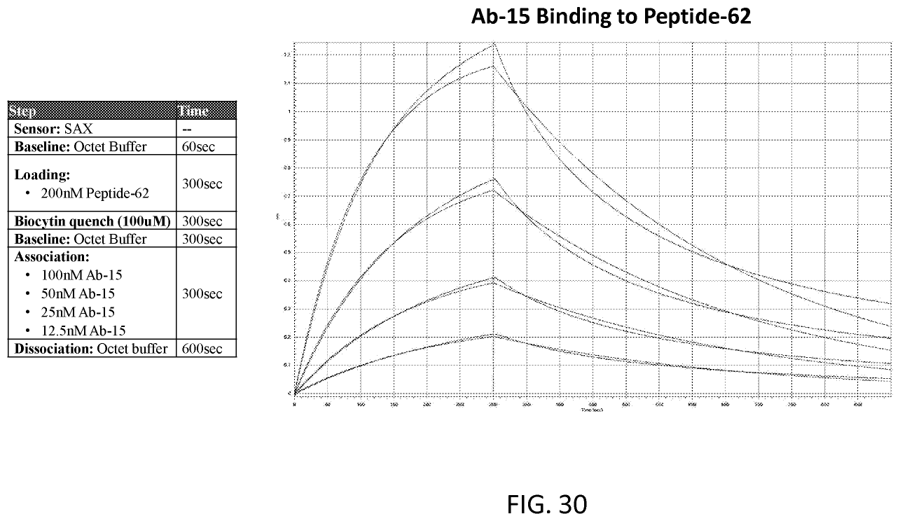

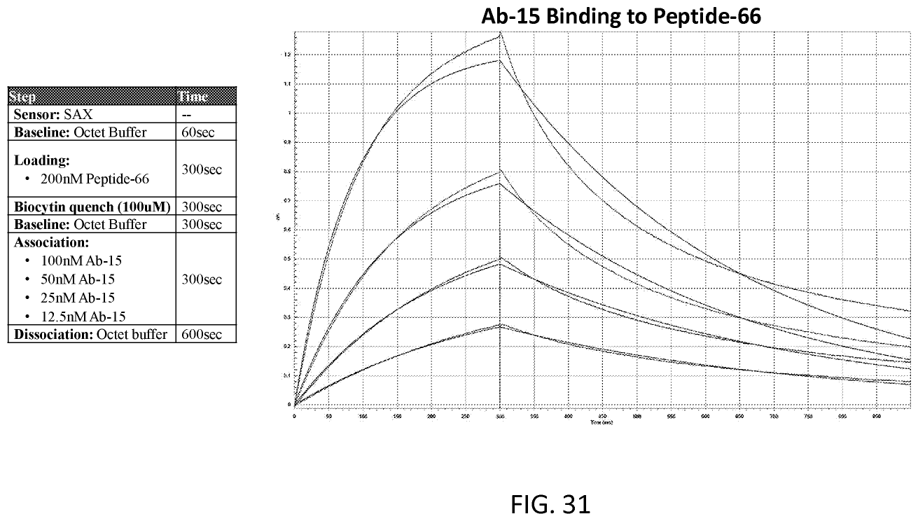

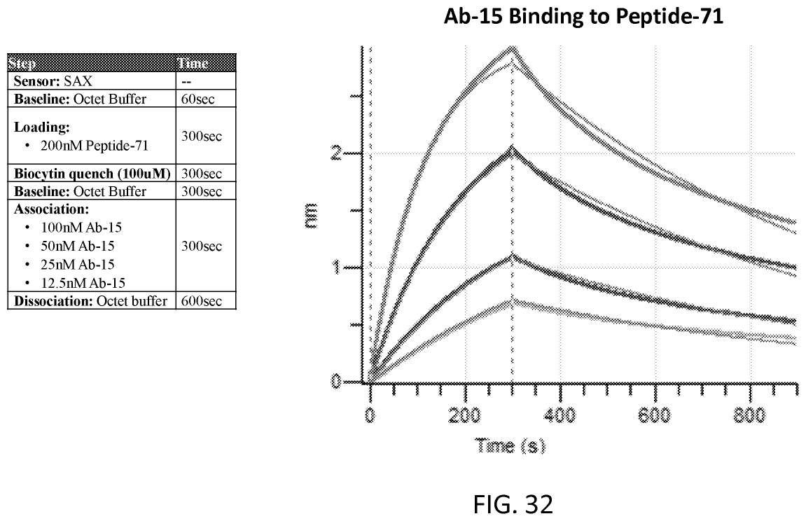

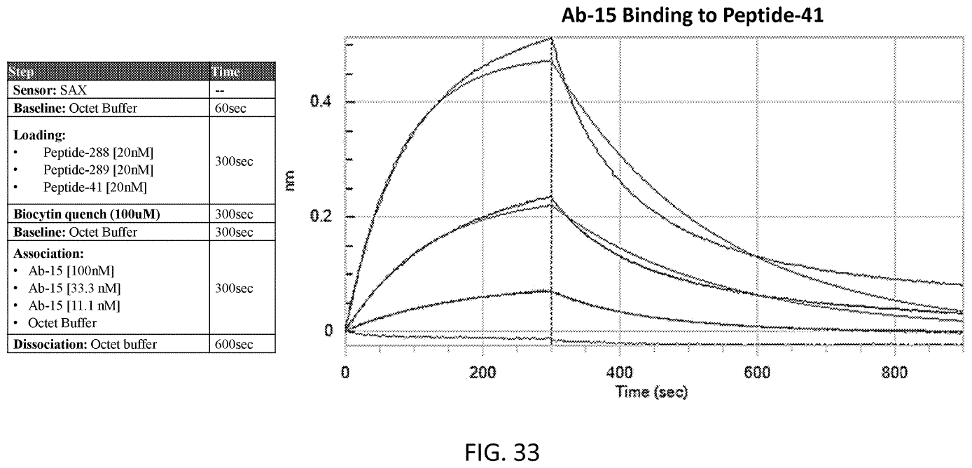

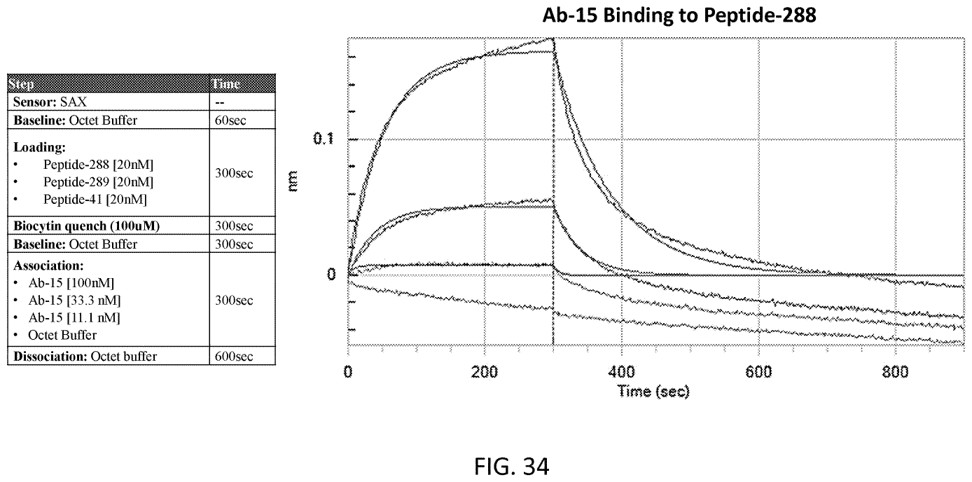

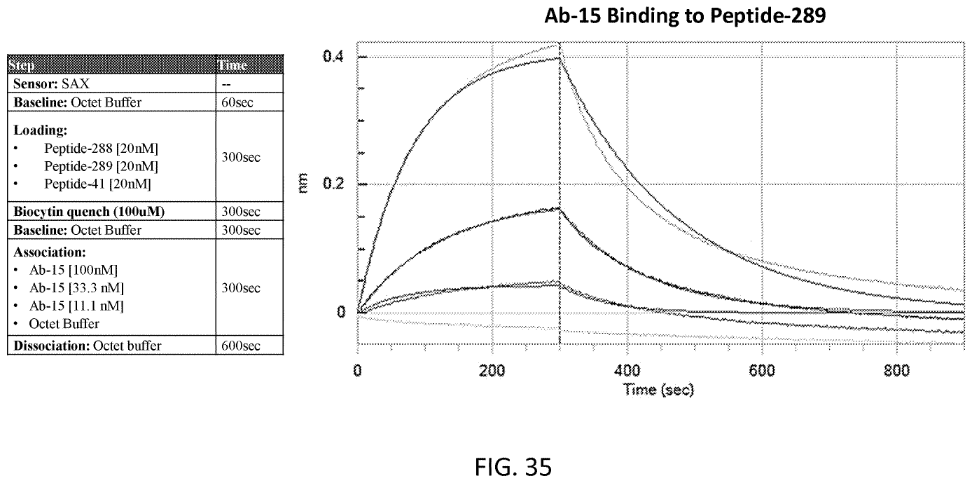

Kinetic binding of the peptides of Table 5 to Ab-15 was evaluated using biolayer interferometry (BLI). Briefly, biotinylated peptides were loaded onto a streptavidin coated Octett SAX biosensor, quenched in biocytin, and baselined in buffer. Ab-15 was titrated in solution and associated onto the peptide loaded sensor. After a short association period, sensors were transferred into buffer and the dissociation of bound Ab-15 was measured. Association and dissociation signals were recorded in real time and analyzed using a 1:1 binding model within the instrument software. Analysis using a 1:1 binding model enabled the calculation of the on and off rate constants as well as affinity, K_{D}. Binding sensorgrams as well as the timing and steps used for the experiments are shown in FIGS. 26-35. K_{D} and half-life (t_{1/2}) values obtained from the kinetic binding measurements are provided in Tables 20-21. Octet signals and t_{1/2} values when using 200 or 20 nanomolar (nM) peptide and 100 nM Ab-15 are provided in Tables 22-23.

### Binding Inhibition by Peptide Masks

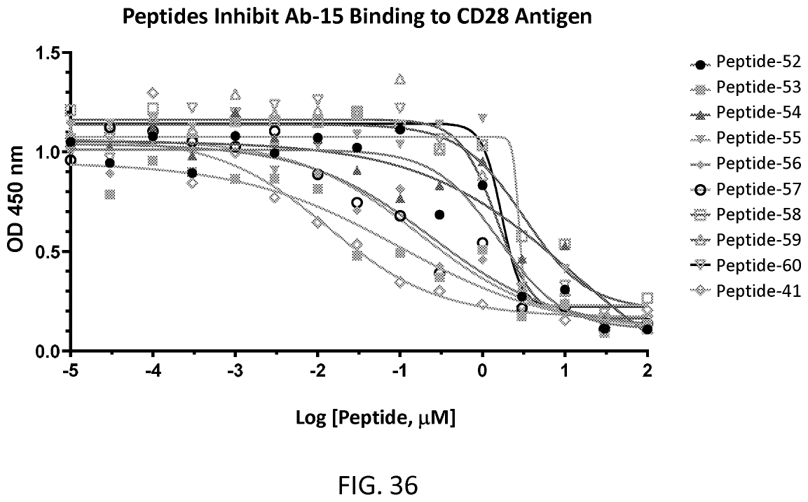

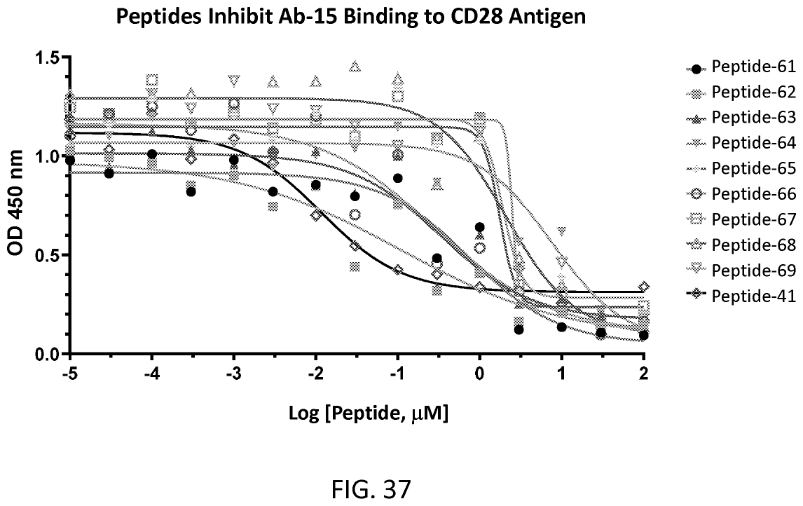

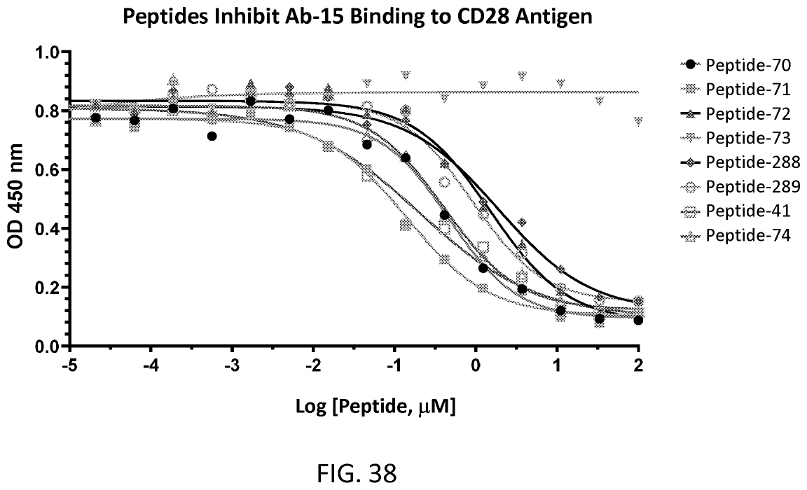

The peptides of Table 5 were further evaluated for their ability to inhibit anti-CD28 scFv harbored in Ab-15 from binding to the CD28 antigen in a standard ELISA format. Briefly. biotinylated CD28 antigen was captured on neutravidin coated plates. Ab-15 fixed at I nanomolar (nM) concentration was pre-incubated with 0-100 micromolar (μM) titrated peptides. After a short pre-incubation period, the mixture of titrated peptide with fixed Ab-15 were added to the CD28 antigen coated plates. After a short incubation on the plates, bound Ab-15 was detected with a standard horse radish peroxidase conjugated secondary antibody. The concentration of peptide required to reduce the maximum signal by 50% (IC₅₀) was calculated in Graphpad Prism software. FIGS. 36-38 illustrate inhibition of Ab-15 binding to CD28 by the peptide masks as measured by ELISA. IC₅₀ₛ are provided in Tables 20-21.

TABLE 20

| | Binding | Competition | | |
|---|---|---|---|---|
| | ELISA | ELISA | K_{D}, | t_{1/2}, |
| Peptide | EC₅₀, nM | IC₅₀, μM | nM | min |
| CD28-biotin | 0.3 | | | |
| Peptide-41 | 4.0 | 0.06 | 15.1 | 15.5 |
| Peptide-52 | 75.9 | 2.00 | | |
| Peptide-53 | 14.6 | 0.17 | 20.3 | 6.2 |
| Peptide-54 | 84.3 | 5.03 | | |
| Peptide-55 | 86.8 | 4.46 | | |
| Peptide-56 | 25.7 | 0.33 | 24.6 | 4.2 |
| Peptide-57 | 32.8 | 0.3 | 23.2 | 3.1 |
| Peptide-58 | 73.9 | 7.06 | | |
| Peptide-59 | 45.4 | 2.18 | | |
| Peptide-60 | 44.9 | 2.66 | | |
| Peptide-61 | 23.4 | 0.63 | | |
| Peptide-62 | 19.7 | 0.19 | 26.1 | 4.5 |
| Peptide-63 | 39.3 | 0.78 | | |
| Peptide-64 | 101.6 | 8.34 | | |
| Peptide-65 | 66.5 | 2.63 | | |
| Peptide-66 | 24.0 | 0.59 | 25.5 | 4.7 |
| Peptide-67 | 21.7 | 2.80 | | |
| Peptide-18 | 32.2 | 3.25 | | |
| Peptide-19 | 32.5 | 4.42 | | |

TABLE 21

| | Binding | Competition | | |
|---|---|---|---|---|
| | ELISA | ELISA | K_{D}, | t_{1/2}, |
| Peptide | EC₅₀, nM | IC₅₀, μM | nM | min |
| CD28-biotin | 0.2 | | | |
| Peptide-41 | 1.0 | 0.2 | | |
| Peptide-288 | 41.7 | 1.89 | | |
| Peptide-289 | 19.6 | 0.91 | | |
| Peptide-70 | 8.2 | 0.47 | | |
| Peptide-71 | 2.1 | 0.14 | 16.0 | 9.1 |
| Peptide-72 | 13.3 | 1.52 | | |
| Peptide-73 | 54.9 | >100 | | |
| Peptide-74 | 7.6 | 0.41 | | |

TABLE 22
Kinetic Binding Data (200 nM peptide; 100 nM Ab-15)

| Peptide | Octet signal | t_{1/2} (min) |
|---|---|---|
| Peptide-41 | 2.0349 | 9.8 |
| Peptide-52 | 0.7003 | 3.1 |
| Peptide-53 | 1.2187 | 4.9 |
| Peptide-54 | 0.4345 | 4.0 |
| Peptide-55 | 0.4608 | 4.2 |
| Peptide-56 | 1.1165 | 3.6 |
| Peptide-57 | 1.0208 | 3.0 |
| Peptide-58 | 0.4787 | 3.9 |
| Peptide-59 | 0.7365 | 3.0 |
| Peptide-60 | 0.8453 | 3.1 |
| Peptide-61 | 1.3654 | 4.5 |
| Peptide-62 | 1.1326 | 4.0 |
| Peptide-63 | 1.0086 | 3.3 |
| Peptide-64 | 0.3275 | 3.4 |
| Peptide-65 | 0.5506 | 2.9 |
| Peptide-66 | 1.3362 | 4.2 |
| Peptide-67 | 1.1498 | 4.1 |
| Peptide-68 | 0.8489 | 2.9 |
| Peptide-69 | 0.9193 | 2.9 |

TABLE 23
Kinetic Binding Data (100 nM Ab-15)

| | Octet signal | Octet signal |
|---|---|---|
| Peptide | (200 nM peptide) | (20 nM peptide) |
| Peptide-41 | 1.8035 | 0.5088 |
| Peptide-288 | | 0.1733 |
| Peptide-289 | | 0.4179 |
| Peptide-70 | 0.6966 | |
| Peptide-71 | 1.524 | |
| Peptide-72 | 0.8384 | |
| Peptide-73 | 0.1661 | |
| Peptide-74 | 0.7053 | |

### Example 8: Anti-CD28 Fab and Anti-CD28 scFv (disulfide) Binding to Peptide Masks

### Equilibrium Binding Via ELISA

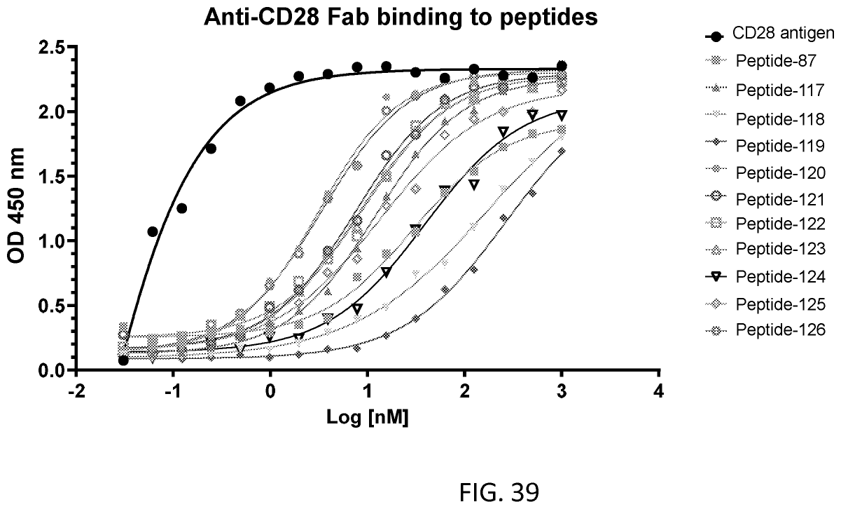

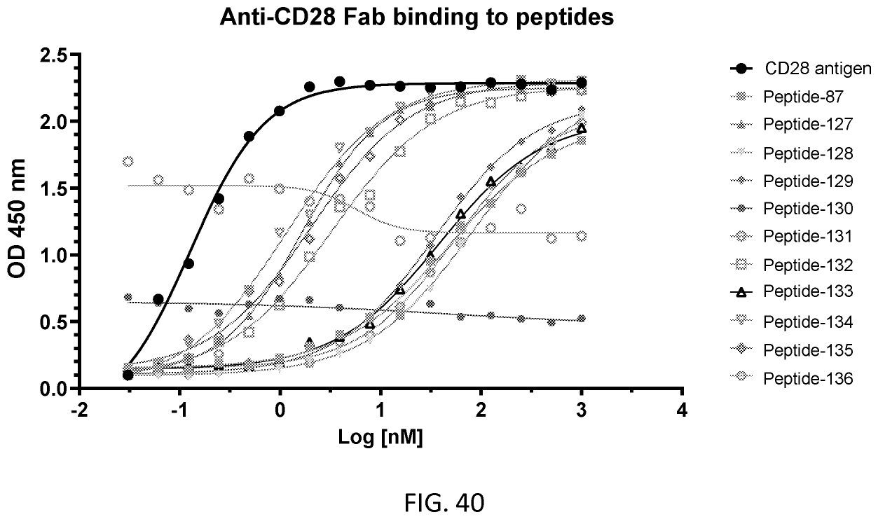

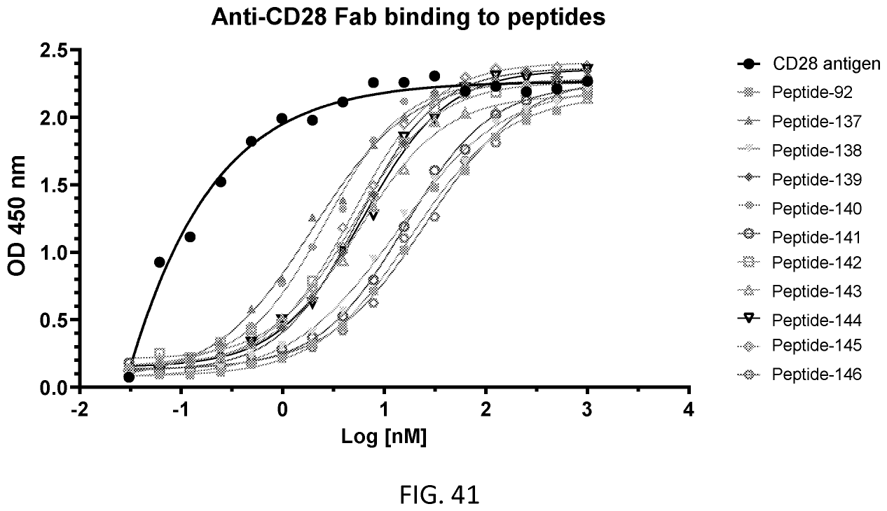

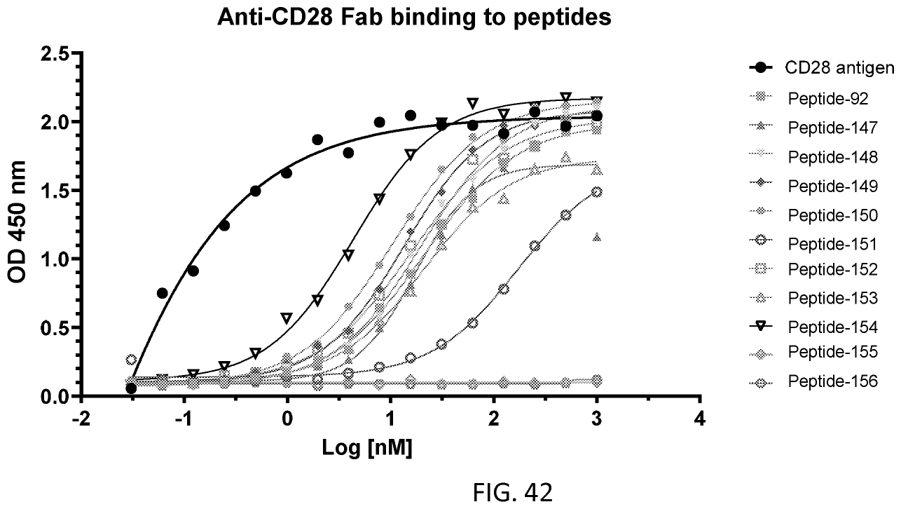

Peptides were evaluated for their ability to bind to ant-CD28 Fab (SEQ ID NOs: 696.697) or anti-CD28 scFv (disulfide) (SEQ ID NO: 10) in a standard ELISA format as described above. Exemplary binding curves for anti-CD28 binding to peptides are shown in FIGS. 39-42. EC₅₀ₛ are shown in Tables 24-27.

### Binding Inhibition by Peptide Masks

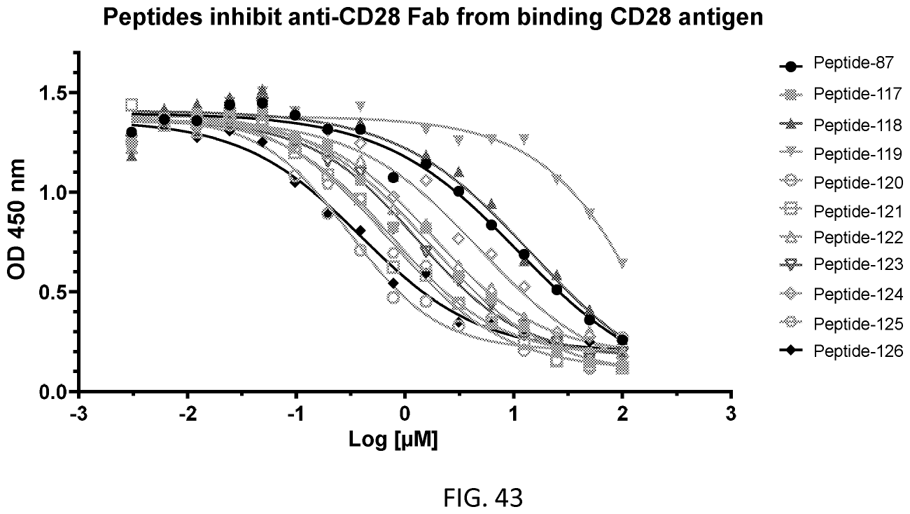

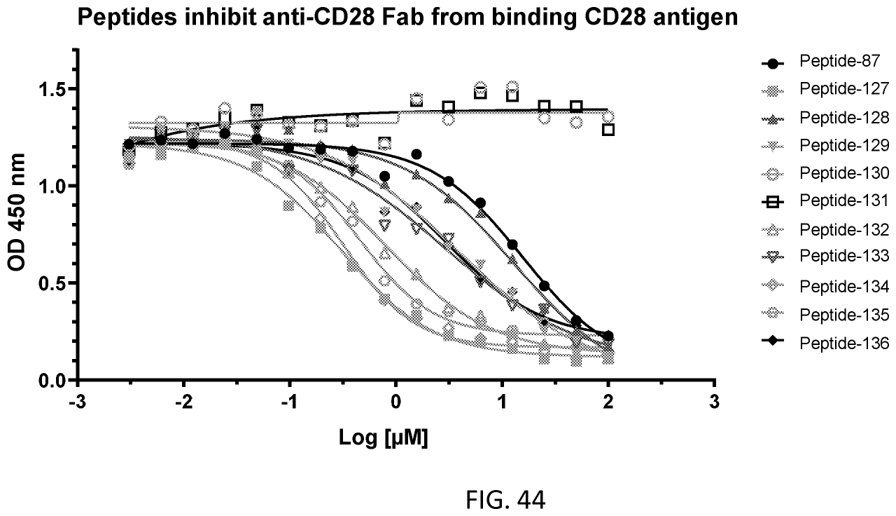

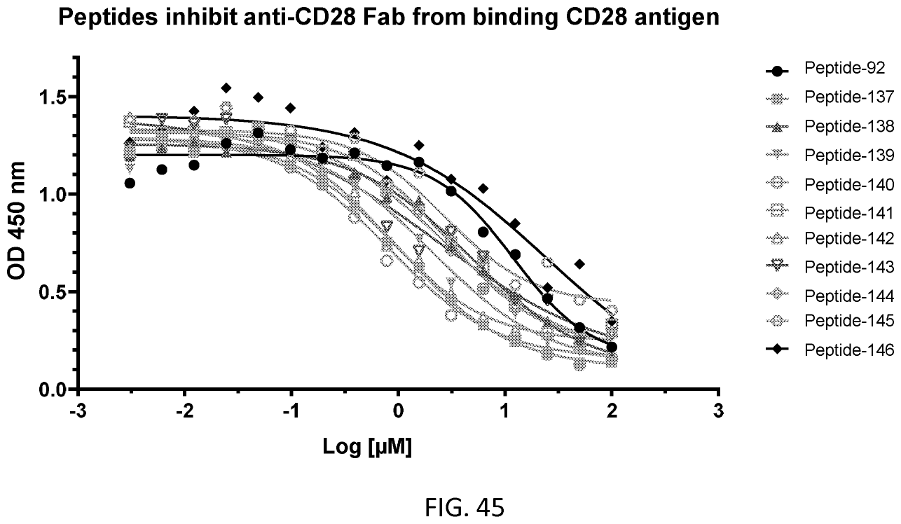

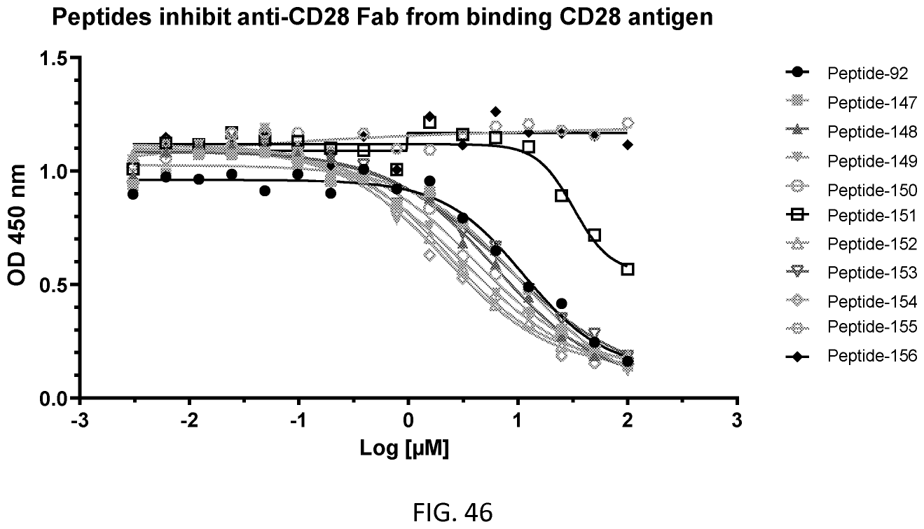

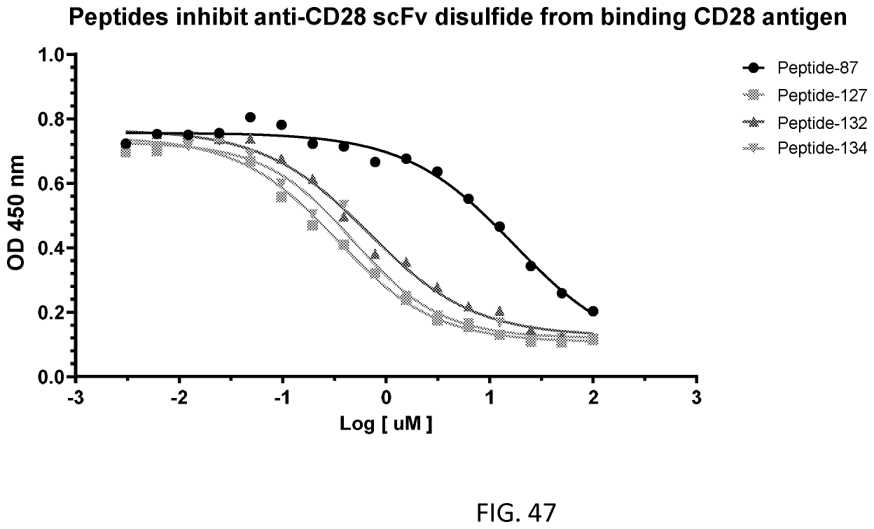

Peptides were further evaluated for their ability to inhibit the an-CD28 Fab (SEQ ID NOs: 696, 697) or to inhibit anti-CD28 scFv (disulfide) (SEQ ID NO: 10) from binding to plate-bound CD28 antigen by competition ELISA experiments as described above. The anti-CD28 Fab was fixed at a concentration of 1 nM and incubated with titrated 0-100 μM peptides. The anti-CD28 scFv was fixed at a concentration of 0.3 nM and incubated with titrated 0-100 μM peptides. FIGS. 43-46 illustrate inhibition of anti-CD28 Fab binding to CD28 by the peptide masks. FIG. 47 illustrates inhibition of anti-CD28 scFv (disulfide) (SEQ ID NO: 10) binding to CD28 by peptide masks. IC-7s are provided in Tables 24-29.

### Kinetic Binding via BLI

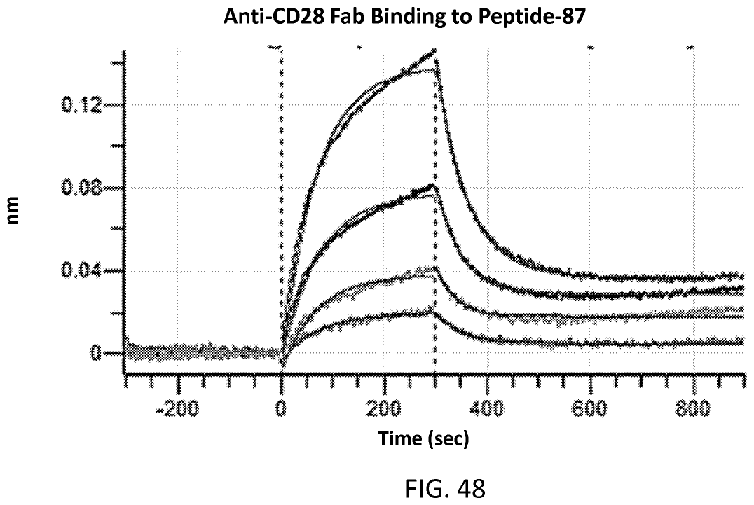

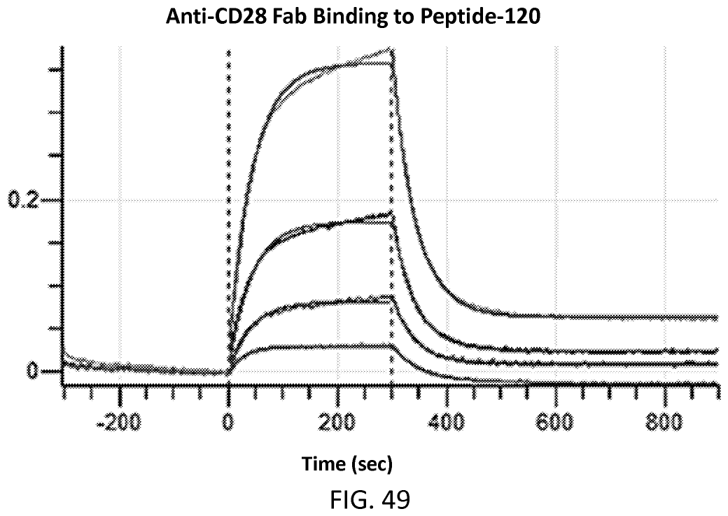

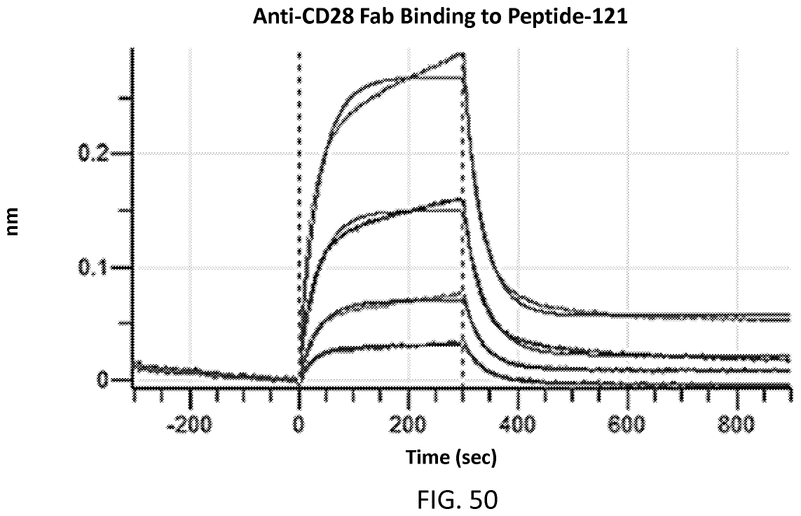

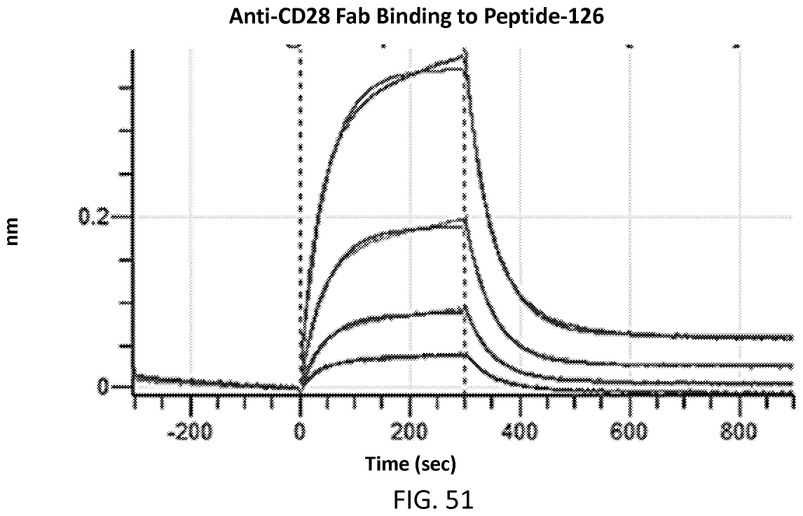

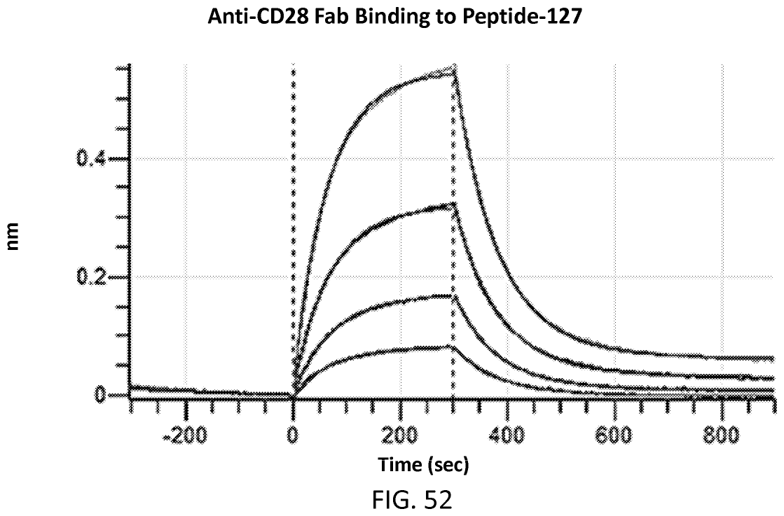

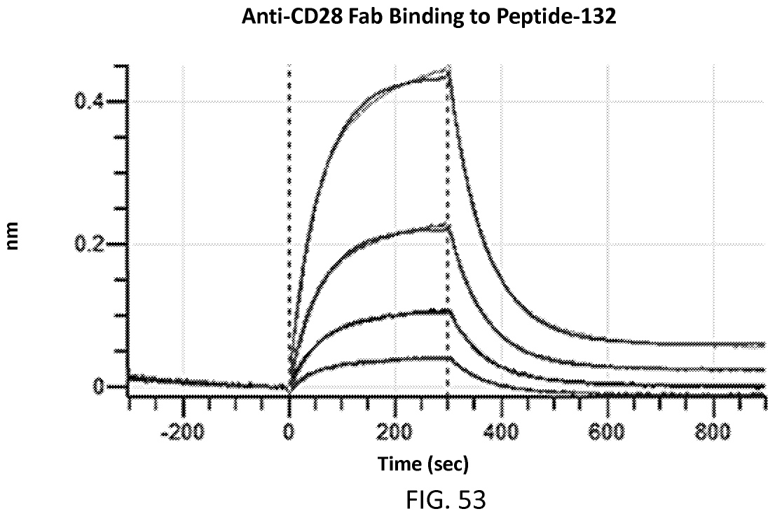

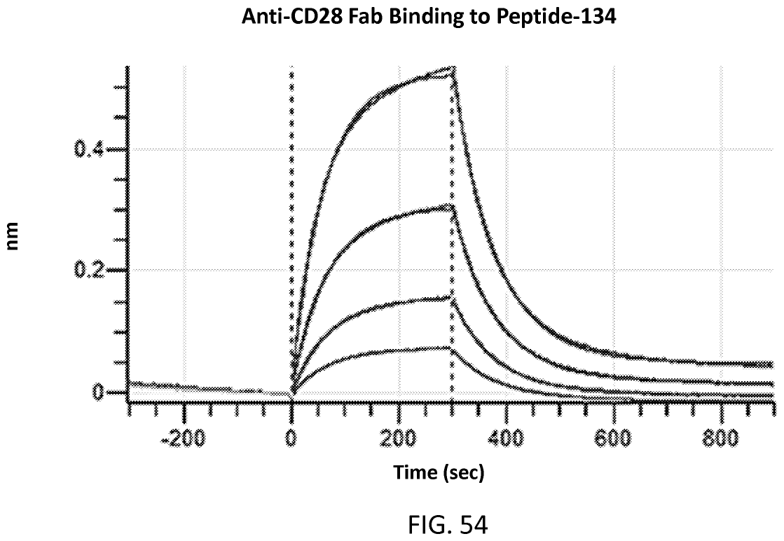

Kinetic binding of the peptides to anti-CD28 Fab (SEQ ID NOs: 696, 697) was evaluated using BLI as described above. FIGS. 48-54 show BLI titration data for binding of various peptides to the anti-CD28 Fab, k_{D}, kₒₙ k_{dis}and t_{1/2} values obtained from the BLI data are provided in Table 30.

TABLE 24

anti-CD28 Fab

| | Binding ELISA | Competition |
|---|---|---|
| Peptide | EC₅₀ nM | ELISA IC₅₀ μM |
| Peptide-41 | 490.0 | >100 |
| Peptide-75 | >1000 | |
| Peptide-76 | >1000 | |
| Peptide-77 | >1000 | |
| Peptide-78 | >1000 | |
| Peptide-79 | 97 | >100 |
| Peptide-80 | >1000 | |
| Peptide-81 | >1000 | |
| Peptide-82 | >1000 | |
| Peptide-83 | >1000 | |
| Peptide-84 | 337 | 26.82 |
| Peptide-85 | >1000 | >100 |
| Peptide-86 | >1000 | |
| Peptide-87 | 49 | 13.77 |
| Peptide-88 | >1000 | |
| Peptide-89 | >1000 | |
| Peptide-90 | >1000 | |
| Peptide-91 | >1000 | |
| Peptide-92 | 54 | 2.39 |
| Peptide-93 | >1000 | |
| Peptide-94 | >1000 | |
| Peptide-95 | | 43.59 |
| Peptide-96 | | 7.59 |
| Peptide-97 | | 69.10 |
| Peptide-98 | | 7.04 |

TABLE 25

anti-CD28 Fab

| | Binding ELISA | Competition |
|---|---|---|
| Peptide | EC₅₀ nM | ELISA IC₅₀ μM |
| Peptide-92 | 18.9 | 11.78 |
| Peptide-137 | 2.1 | 0.89 |
| Peptide-138 | 13.1 | 3.61 |
| Peptide-139 | 5.3 | 2.19 |
| Peptide-140 | 2.6 | 0.76 |
| Peptide-141 | 15.7 | 2.22 |
| Peptide-142 | 4.5 | 0.62 |
| Peptide-143 | 5.6 | 2.56 |
| Peptide-144 | 5.6 | 3.04 |
| Peptide-145 | 4.4 | 7.70 |
| Peptide-146 | 24.3 | 16.12 |
| Peptide-147 | 25.8 | 8.15 |
| Peptide-148 | 19.0 | 5.67 |
| Peptide-149 | 12.8 | 2.66 |
| Peptide-150 | 10.6 | 4.76 |
| Peptide-151 | 112.4 | 80.52 |
| Peptide-152 | 15.6 | 2.22 |
| Peptide-153 | 20.5 | 8.42 |
| Peptide-154 | 4.2 | 2.20 |
| Peptide-155 | >1000 | >100 |
| Peptide-156 | >1000 | >100 |

TABLE 26

anti-CD28 Fab

| | Binding ELISA | Competition | Off rate |
|---|---|---|---|
| Peptide | EC₅₀ nM | ELISA IC₅₀ μM | t½ (min) |
| Peptide-87 | 30.6 | 9.76 | 0.7 |
| Peptide-117 | 11.0 | 1.53 | |
| Peptide-118 | 65.8 | 13.33 | |
| Peptide-119 | 127.7 | 77.71 | |
| Peptide-120 | 3.1 | 0.36 | 0.5 |
| Peptide-121 | 7.1 | 0.48 | 0.4 |
| Peptide-122 | 7.8 | 2.22 | |
| Peptide-123 | 8.1 | 1.32 | |
| Peptide-124 | 28.1 | 4.07 | |
| Peptide-125 | 12.1 | 0.83 | |
| Peptide-126 | 3.4 | 0.49 | 0.6 |
| Peptide-127 | 1.7 | 0.30 | 1.0 |
| Peptide-128 | 59.4 | 9.66 | |
| Peptide-129 | 30.5 | 3.18 | |
| Peptide-130 | >1000 | >100 | |
| Peptide-131 | >1000 | >100 | |
| Peptide-132 | 3.2 | 0.35 | 0.8 |
| Peptide-133 | 32.5 | 2.90 | |
| Peptide-134 | 1.3 | 0.37 | 0.9 |
| Peptide-135 | 2.1 | 0.63 | 0.9 |
| Peptide-136 | 44.9 | 4.12 | |

TABLE 27

| | anti-CD28 scFv (disulfide) | |
|---|---|---|
| Peptide | Binding ELISA EC₅₀ nM | Competition ELISA IC₅₀ μM |
| Peptide-41 | 98.5 | 61.53 |
| Peptide-75 | >1000 | |
| Peptide-76 | >1000 | |
| Peptide-77 | >1000 | |
| Peptide-78 | >1000 | |
| Peptide-79 | 73.17 | >100 |
| Peptide-80 | >1000 | |
| Peptide-81 | >1000 | |
| Peptide-82 | >1000 | |
| Peptide-83 | >1000 | |
| Peptide-84 | >1000 | >100 |
| Peptide-85 | >1000 | >100 |
| Peptide-86 | >1000 | |
| Peptide-87 | 155.90 | >100 |
| Peptide-88 | >1000 | |
| Peptide-89 | >1000 | |
| Peptide-90 | >1000 | |
| Peptide-91 | >1000 | |
| Peptide-92 | >1000 | >100 |
| Peptide-93 | >1000 | |
| Peptide-94 | >1000 | |

TABLE 28

| | anti-CD28 Fab |
|---|---|
| Peptide | Competition ELISA IC₅₀ μM |
| Peptide-92 | 7.89 |
| Peptide-99 | 20.98 |
| Peptide-100 | >100 |
| Peptide-101 | 7.42 |
| Peptide-102 | >100 |
| Peptide-103 | >100 |
| Peptide-104 | >100 |
| Peptide-105 | 12.95 |
| Peptide-106 | 76.43 |
| Peptide-87 | 12.56 |
| Peptide-107 | >100 |
| Peptide-108 | >100 |
| Peptide-109 | >100 |
| Peptide-110 | 3.16 |
| Peptide-111 | 12.29 |
| Peptide-112 | >100 |
| Peptide-113 | 102.00 |
| Peptide-114 | 5.49 |
| Peptide-115 | >100 |
| Peptide-116 | 98.64 |

TABLE 29

| | anti-CD28 scFv (disulfide) |
|---|---|
| Peptide | Competition ELISA IC₅₀ μM |
| Peptide-92 | 31.18 |
| Peptide-99 | 67.05 |
| Peptide-100 | >100 |
| Peptide-101 | 36.18 |
| Peptide-102 | >100 |
| Peptide-103 | >100 |
| Peptide-104 | >100 |
| Peptide-105 | 56.91 |
| Peptide-106 | >100 |
| Peptide-87 | 10.99 |
| | 14.64 |
| Peptide-107 | >100 |
| Peptide-108 | >100 |
| Peptide-109 | >100 |
| Peptide-110 | 1.91 |
| Peptide-111 | 10.48 |
| Peptide-112 | >100 |
| Peptide-113 | >100 |
| Peptide-114 | 7.09 |
| Peptide-115 | >100 |
| Peptide-116 | 76.41 |
| Peptide-127 | 0.35 |
| Peptide-132 | 0.78 |
| Peptide-134 | 0.48 |

TABLE 30

| Peptide | K_{D} (M) | kₒₙ (1/Ms) | k_{dis} (1/s) | t_{1/2} (min) |
|---|---|---|---|---|
| Peptide-87 | 2.02E−06 | 1.00E+04 | 2.02E−02 | 0.6 |
| Peptide-120 | 9.26E−07 | 2.58E+04 | 2.39E−02 | 0.5 |
| Peptide-121 | 8.31E−07 | 3.37E+04 | 2.80E−02 | 0.4 |
| Peptide-126 | 1.41E−07 | 1.35E+05 | 1.91E−02 | 0.6 |
| Peptide-127 | 4.47E−08 | 2.64E+05 | 1.18E−02 | 1.0 |
| Peptide-132 | 1.10E−07 | 1.25E+05 | 1.38E−02 | 0.8 |
| Peptide-134 | 4.02E−08 | 2.86E+05 | 1.15E−02 | 1.0 |

### Example 9: Sequence Activity Relationships—CD28 Masks

Sequence activity relationships (SAR) were established for peptide-87 and peptide-92 which were found to exhibit strong binding to the anti-CD28 Fab. The sequence activity relationships were established by mutating individual residues within each peptide to alanine and measuring the influence of each mutation on the ability of the peptide to inhibit the anti-CD28 Fab (SEQ ID NOs: 696, 697) from binding to CD28 by competition ELISA experiments as described above in Example 1. IC₅₀ₛ for binding inhibition by the alanine scanning sequences are provided in Tables 31-32.

TABLE 31
Competition Binding ELISA - Alanine Scanning Sequences of Peptide-87

| | | | Competition ELISA |
|---|---|---|---|
| Peptide | SEQ ID NO: | Sequence (N to C) | IC₅₀ (mM) |
| Peptide-87 | 744 | YCAGHAWQYINKCL | 12.56 |
| Peptide-107 | 764 | ACAGHAWQYINKCL | >100 |
| Peptide-108 | 765 | YCAAHAWQYINKCL | >100 |
| Peptide-109 | 766 | YCAGAAWQYINKCL | >100 |
| Peptide-110 | 767 | YCAGHAAQYINKCL | 3.16 |
| Peptide-111 | 768 | YCAGHAWAYINKCL | 12.29 |
| Peptide-112 | 769 | YCAGHAWQAINKCL | >100 |
| Peptide-113 | 770 | YCAGHAWQYANKCL | 102.00 |
| Peptide-114 | 771 | YCAGHAWQYIAKCL | 5.49 |
| Peptide-115 | 772 | YCAGHAWQYINACL | >100 |
| Peptide-116 | 773 | YCAGHAWQYINKCA | 98.64 |

TABLE 32
Competition Binding ELISA - Alanine Scanning Sequences of Peptide-92

| | | | Competition ELISA |
|---|---|---|---|
| Peptide | SEQ ID NO: | Sequence (N to C) | IC₅₀ (mM) |
| Peptide-92 | 749 | SCAAGASYAWPYCL | 7.89 |
| Peptide-100 | 756 | ACAAGASYAWPYCL | 20.98 |
| Peptide-101 | 757 | SCAAAASYAWPYCL | >100 |
| Peptide-101 | 758 | SCAAGAAYAWPYCL | 7.42 |
| Peptide-102 | 759 | SCAAGASAAWPYCL | >100 |
| Peptide-103 | 760 | SCAAGASYAAPYCL | >100 |
| Peptide-104 | 761 | SCAAGASYAWAYCL | >100 |
| Peptide-105 | 762 | SCAAGASYAWPACL | 12.95 |
| Peptide-106 | 763 | SCAAGASYAWPYCA | 76.43 |

### Example 10: Optimized Phage Library Construction of Peptide-87

Using the peptide sequence activity relationships of Example 9, DNA oligo libraries were constructed where codons encoding critical residues within peptide-87 were minimally mutated, and codons encoding non-critical residues that were tolerated in the alanine scanning mutation studies (e.g., Trp-7, Gln-8, and Asn-11) were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field.

### Example 11: Panning of Peptide-87 Optimized Phage Library

Once the phage optimization libraries were completed, phage libraries were bio-panned using CD28 Fab loaded beads. Multiple rounds of panning were performed where bacteriophage was allowed to bind to CD28 Fab loaded beads, washed, eluted, and amplified. After panning, phage infected bacteria were plated out and colonies picked into 96 well blocks. Clonal phage was then amplified and separated from bacterial cells via centrifugation. Phage containing supernatants were tested in binding ELISAs against CD28 Fab coated plates in the presence or absence of saturating concentrations of CD28 antigen. Phage able to bind CD28 Fab were selected for sequence analysis if the binding signal was reduced in the presence of CD28 antigen.

### Example 12: Panning ELISAs-CD28 Fab Peptides

Clonal phage were harvested as crude supematants and screened via standard ELISAs. Briefly, biotinylated CD28 Fab was captured on neutravidin coated plates. Prior to the addition of clonal phage, wells were incubated with blocking buffer and CD28 antigen or blocking buffer alone. Without washing or aspirating, clonal phage supematants were then added to the wells and incubated for a short time. Wells were then washed followed by detection of bound phage using a horse radish conjugated anti-M13 antibody. Clonal phage of interest were then sent for sequence analysis.

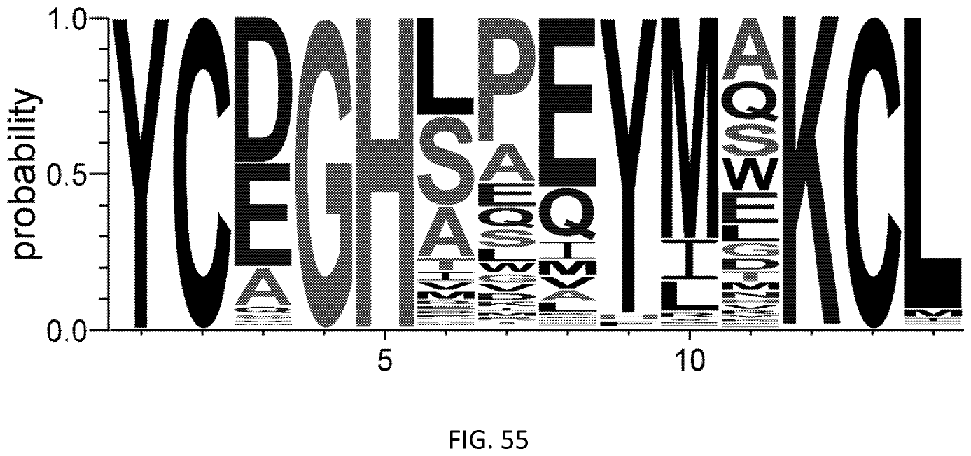

Phage panning results of peptide-87 library sequences are shown in Table 7. 886 clonal phage sequences were identified. A consensus sequence was calculated from all the sequences of Table 7 using WebLogo 3.7.12. The consensus sequence is shown in FIG. 55.

### Example 13: Optimized Phage Library Construction of Peptide-92

Using the peptide sequence activity relationships of Example 9, DNA oligo libraries were constructed where codons encoding critical residues within peptide-92 were minimally mutated, and codons encoding non-critical residues that were tolerated in the alanine scanning mutation studies (e.g., Ser-1, Gly-5, Ser-7, Tyr-8, Try-12) were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field.

### Example 14: Panning of the peptide-92 Optimized Phage Library

Once the phage optimization libraries were completed, phage libraries were bio-panned using CD28 Fab loaded beads. Multiple rounds of panning were performed where bacteriophage was allowed to bind to CD28 Fab loaded beads, washed, eluted, and amplified. After panning, phage infected bacteria were plated out and colonies picked into 96 well blocks. Clonal phage was then amplified and separated from bacterial cells via centrifugation. Phage containing supematants were tested in binding ELISAs against CD28 Fab coated plates in the presence or absence of saturating concentrations of CD28 antigen. Phage able to bind CD28 Fab were selected for sequence analysis if the binding signal was reduced in the presence of CD28.

### Example 15: Panning ELISAs—CD28 Fab Peptides

Clonal phage were harvested as crude supematants and screened via standard ELISAs. Briefly, biotinylated CD28 Fab was captured on neutravidin coated plates. Prior to the addition of clonal phage, wells were incubated with blocking buffer and CD28 antigen or blocking buffer alone. Without washing or aspirating, clonal phage supematants were then added to the wells and incubated for a short time. Wells were then washed followed by detection of bound phage using a horse radish conjugated anti-M13 antibody. Clonal phage of interest were then sent for sequence analysis.

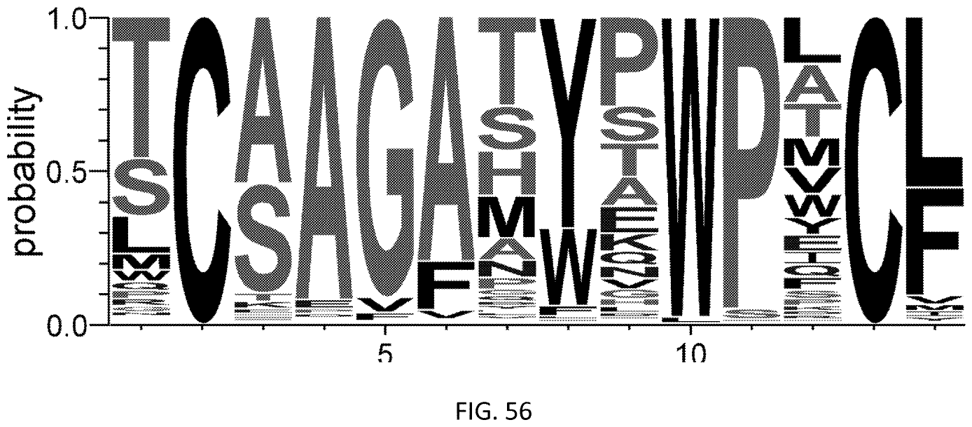

Phage panning results of peptide-92 library sequences are shown in Table 8. 397 clonal phage sequences were identified. A consensus sequence was calculated from all the sequences of Table 8 using WebLogo 3.7.12. The consensus sequence is shown in FIG. 56.

### Example 16. Discovery of Peptides that Bind to Anti-CD28 scFv

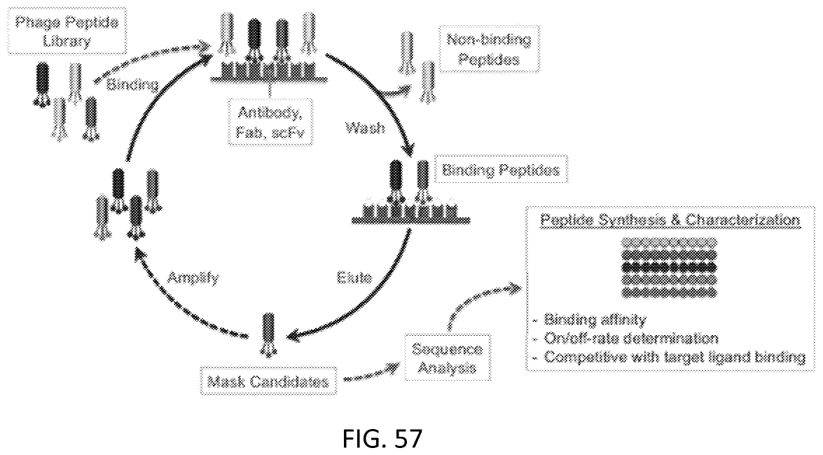

Lead peptides that mask the anti-CD28 scFv according to SEQ ID NO: 9 were identified by phage display according to the method of FIG. 57. Lead hits were then synthesized as peptides and evaluated as described below. Synthetic peptides were evaluated for their ability to bind human anti-CD28 scFv (SEQ ID NO: 9) in a standard enzyme linked immunosorbent assay (ELISA) format. Briefly, biotinylated peptides were captured on neutravidin coated plates. Anti-CD28 scFv diluted in buffer was then added to the peptide captured plates. Bound anti-CD28 scFv was detected using a standard horse radish peroxidase conjugate secondary antibody. The concentration of anti-CD28 scFv required to achieve 50% maximal signal (EC50) was calculated using Graphpad Prism software. Peptides were also evaluated for their ability to inhibit anti-CD28 scFv from binding its cognate antigen, CD28. Briefly, biotinylated CD28 antigen was captured on neutravidin coated plates. Anti-CD28 scFv at 2 nM was pre-incubated with 0-100 pM titrated peptides. After a short pre-incubation period the mixture of titrated peptides with fixed anti-CD28 scFv (2 nM) was added to the CD28 antigen captured plates. After a short incubation on the plates, bound anti-CD28 scFv was detected with a standard horse radish peroxidase conjugated secondary antibody. The concentration of peptide required to reduce the max signal by 50% (IC50) was calculated in Graphpad Prism software.

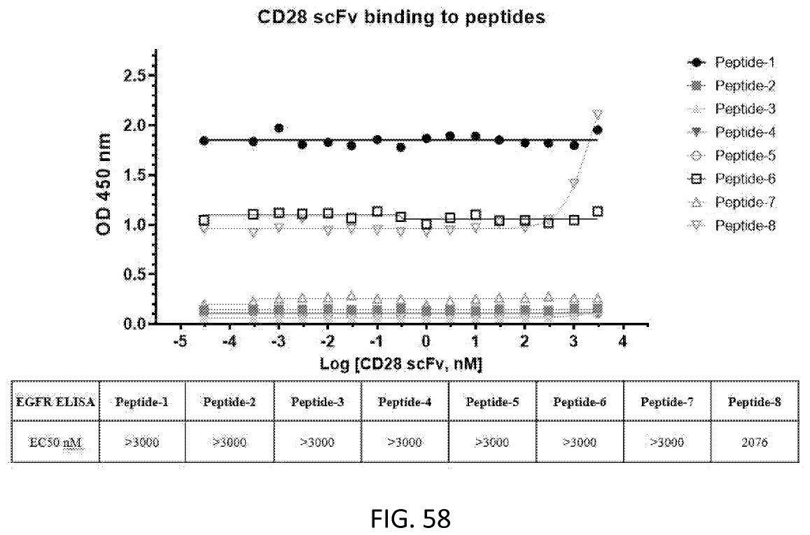

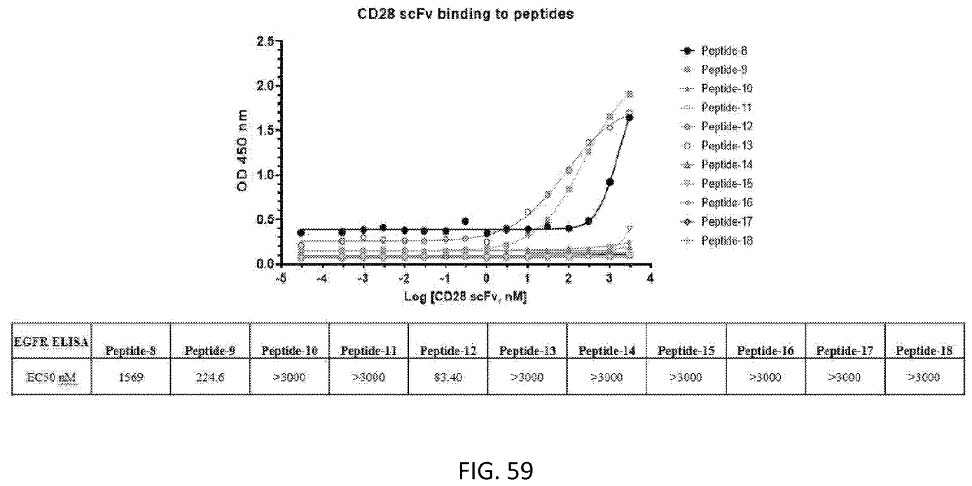

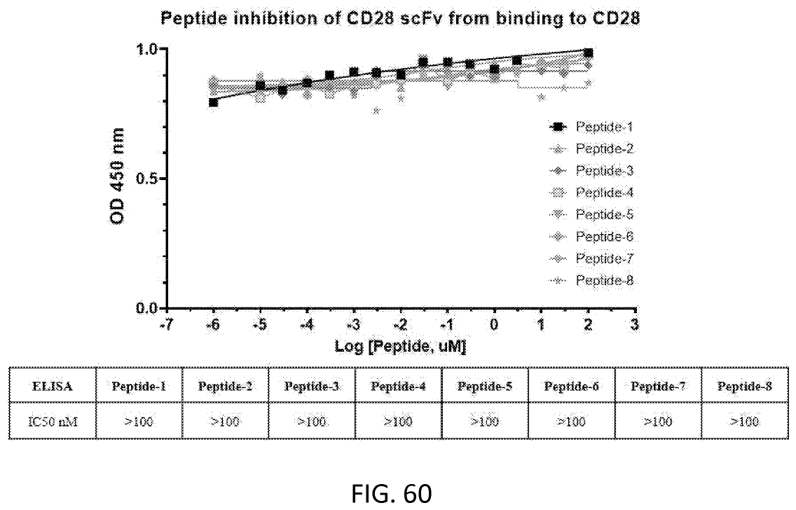

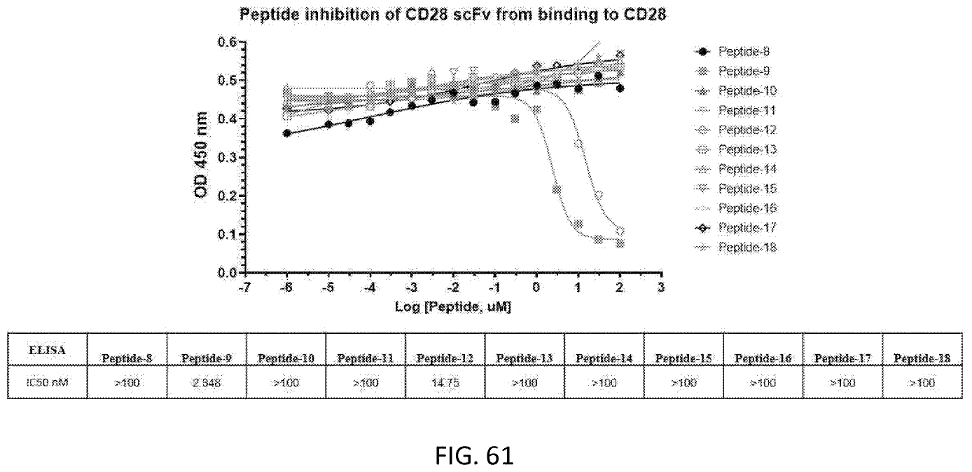

FIG. 58 illustrates anti-CD28 scFv (SEQ ID NO: 9) binding to peptides measured by ELISA. FIG. 59 illustrates anti-CD28 scFv binding to peptides measured by ELISA. FIGS. 60-61 illustrate that peptides inhibit anti-CD28 scFv from binding to CD28 antigen as measured by ELISA.

### Example 17. Kinetic Binding Assays of anti-CD28 scFv (SEO ID NO: 9) to Peptides-9 and -12

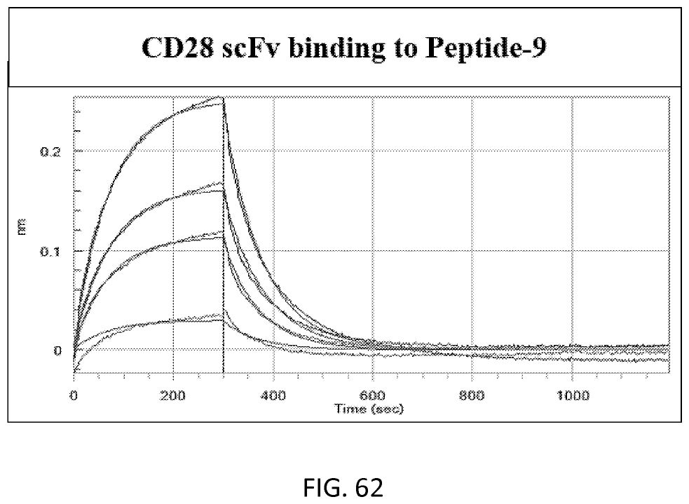

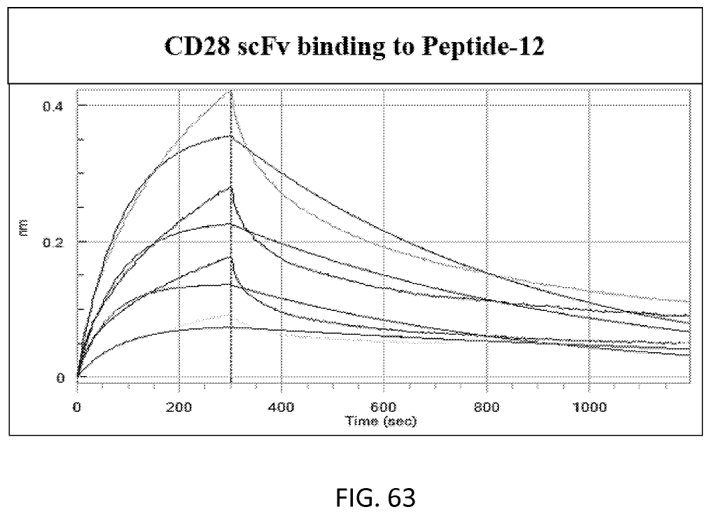

This Example assesses binding of anti-CD28 scFv to Peptide-9 and Peptide-12 in an in vitro kinetic binding assay. Kinetic binding of anti-CD28 scFv to Peptide-9 and Peptide-12 were evaluated by bio-layer interferometry using an Octet RED96 instrument. Briefly, streptavidin biosensors were loaded with biotinylated peptides and baselined in buffer. Anti-CD28 scFv were titrated in solution at 100 nM, 50 nM, 25 nM, and 12.5 nM, then associated onto the peptide loaded sensors. After a short association period, sensors were transferred into buffer and the dissociation of bound anti-CD28 scFv was measured. The timing and steps of the experiment are shown in the accompanying table. Association and dissociation signals were recorded in real time and analyzed using a 1:1 binding model within the instrument software. Analysis using a 1:1 binding model enabled the calculation of the on and off rate constants as well as affinity, K_{D}. Peptide-9 and peptide-12 kinetic binding sensorgrams are shown in FIGS. 62-63 and are summarized in Tables 33-34.

TABLE 33
Timing and Steps of Assay

| Step | Time | |
|---|---|---|
| Baseline: Buffer | 60 | sec |
| Load: | 300 | sec |
| 200 nM Peptide-9 | | |
| 200 nM Peptide-12 | | |
| Baseline: Buffer | 300 | sec |
| Association in octet buffer | 300 | sec |
| 100 nM CD28 scFv | | |
| 50 nM CD28 scFv | | |
| 25 nM CD28 scFv | | |
| 12.5 nM CD28 scFv | | |
| Dissociation: Buffer | 900 | sec |

TABLE 34
Binding Kinetics Summary of anti-CD28
scFv to Peptide-9 and Peptide-12

| Binding domain | Peptide | K_{D} (M) | kon(1/Ms) | kdis(1/s) |
|---|---|---|---|---|
| CD28 scFv | Peptide-9 | 3.77E−07 | 3.25E+04 | 1.23E−02 |
| CD28 scFv | Peptide-12 | 1.70E−08 | 9.87E+04 | 1.67E−03 |

### Example 18: Optimized Phage library construction—anti-CD28 scFv (SEO ID NO: 9) Peptide-9

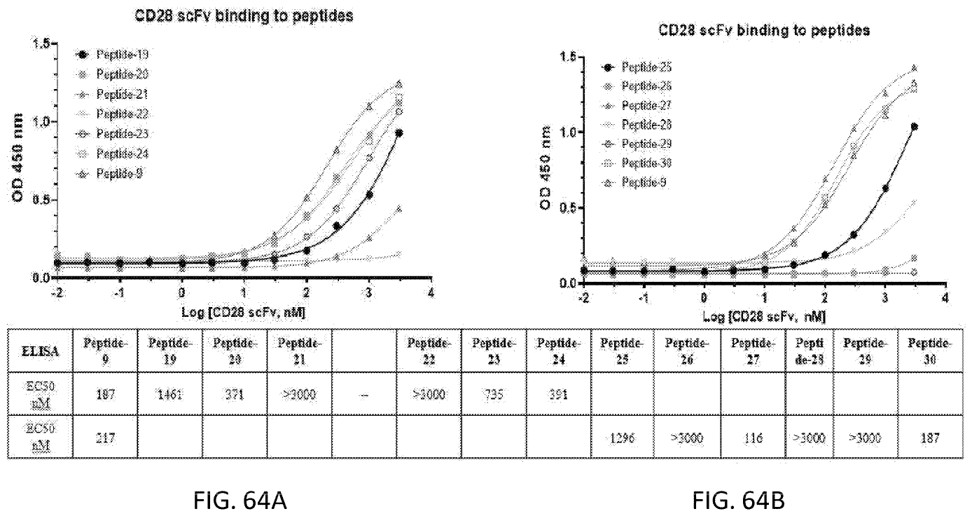

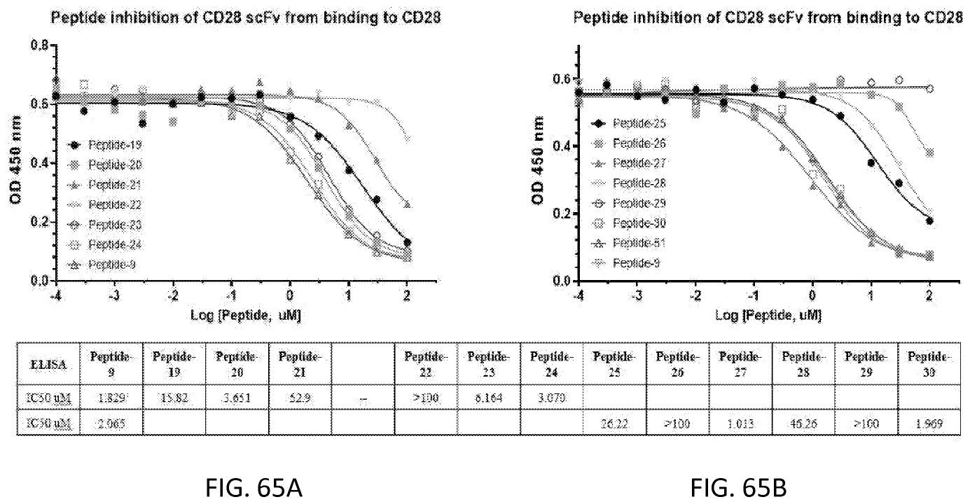

Sequence activity relationships were established for Peptide-9 by mutating each individual residue within the peptide to alanine and measuring binding and inhibition against anti-CD28 scFv. Peptide residues whose alanine mutations significantly weakened binding and inhibition were considered key residues where mutations were not tolerated. Peptide residues whose alanine mutations performed similarly to the non-mutated sequence were considered non-critical sites where mutations were indeed tolerated. Using the peptide sequence activity relationships (SAR), DNA oligo libraries were constructed where codons encoding critical residues within each peptide sequence were minimally mutated and codons encoding non-critical residues were heavily mutated. The resulting oligos were cloned into bacteriophage vectors used to display the SAR guided peptides via fusion to the pill filament of the bacteriophage. The relevant vectors were then used to produce the phage optimization libraries via amplification in bacteria using standard techniques in the field. FIG. 64A and FIG. 64B demonstrate anti-CD28 scFv binding of alanine scanning peptides of Peptide-9 according to the ELISA protocol of Example 16. FIG. 65A and FIG. 65B demonstrate anti-CD28 scFv inhibition of alanine scanning peptides of Peptide-9 according to the ELISA protocol of Example 16.

### Example 19: Panning ELISAs—Anti-CD28 scFv Peptides

Clonal phage were harvested as crude supernatants and screened via standard enzyme linked immunosorbent assays (ELISAs). Briefly, biotinylated anti-CD28 scFv was captured on neutravidin coated plates. Prior to the addition of clonal phage, wells were incubated with blocking buffer and CD28 soluble protein or blocking buffer alone. Without washing or aspirating, clonal phage supernatants were then added to the wells and incubated for a short time. Wells were then washed followed by detection of bound phage using a horse radish peroxidase conjugated anti-M13 antibody. Clonal phage of interest were then sent for sequence analysis.

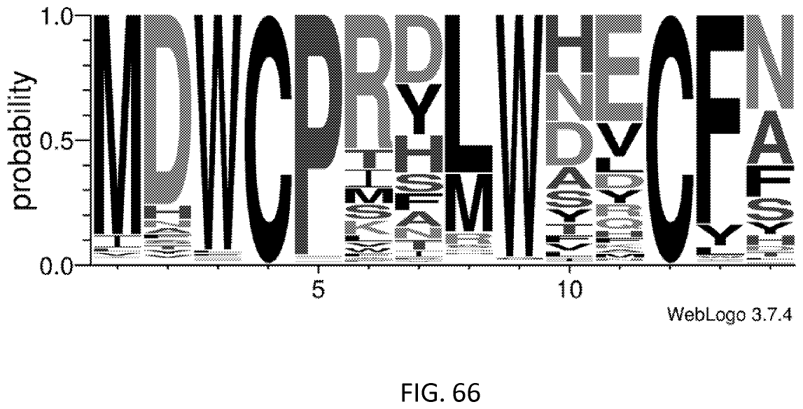

Phage panning results of anti-CD28 scFv Peptide-9 library sequences are shown in Table 6. 453 clonal phage sequences were identified. The consensus sequence calculated from all the sequences of Table 6 is shown in FIG. 66 and was generated using WebLogo 3.7.4.

### Example 20. Peptides Inhibit Anti-CD28 scFv from Binding CD28 Antigen by ELISA

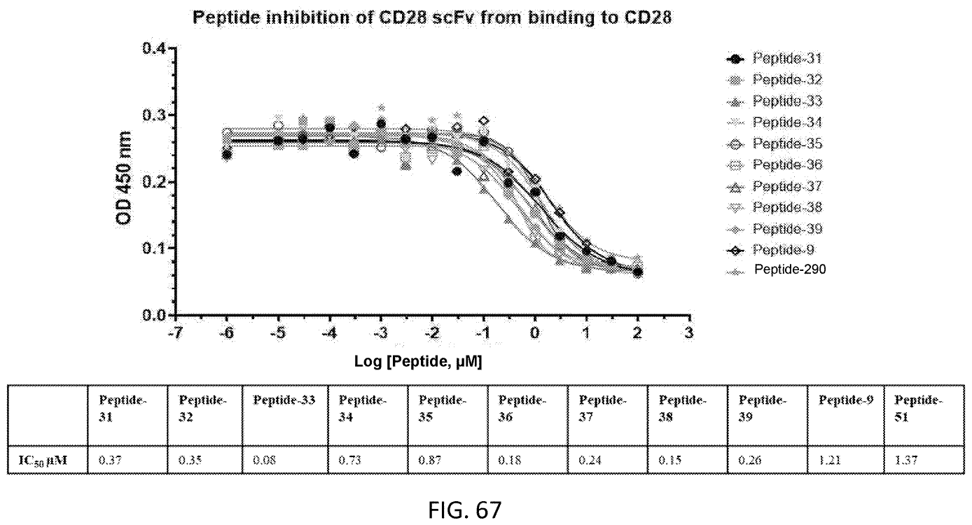

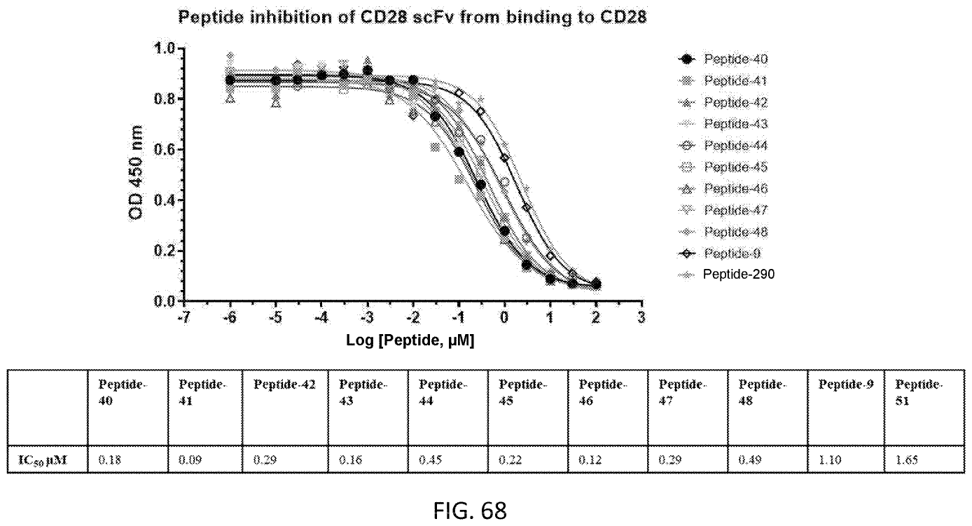

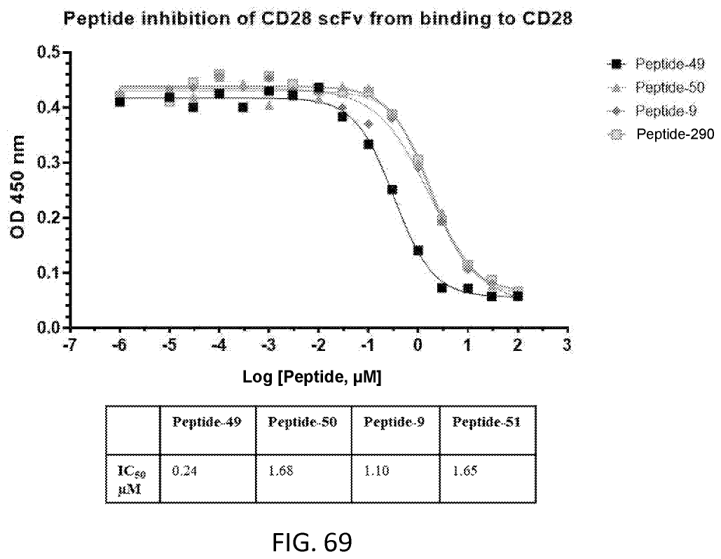

Peptides were evaluated for their ability to inhibit the anti-CD28 scFv (SEQ ID NO: 9) from binding to the CD28 antigen in a standard enzyme linked immunosorbent assay (ELISA) format. Briefly, biotinylated CD28 antigen was captured on neutravidin coated plates. Anti-CD28 scFv at 2 nM were pre-incubated with 0-100 pM titrated peptides. After a short pre-incubation period the mixture of titrated peptides with fixed anti-CD28 scFv (2 nM) were added to the CD28 antigen captured plates. After a short incubation on the plates, bound anti-CD28 scFv was detected with a standard horse radish peroxidase conjugated secondary antibody. The concentration of peptide required to reduce the max signal by 50% (IC₅₀) was calculated in Graphpad Prism software. FIGS. 67-69 illustrate peptides that inhibit the anti-CD28 scFv from binding the CD28 antigen measured by ELISA.

### Example 21. Anti-CD28 scFv Kinetic Binding to Peptides by Octet

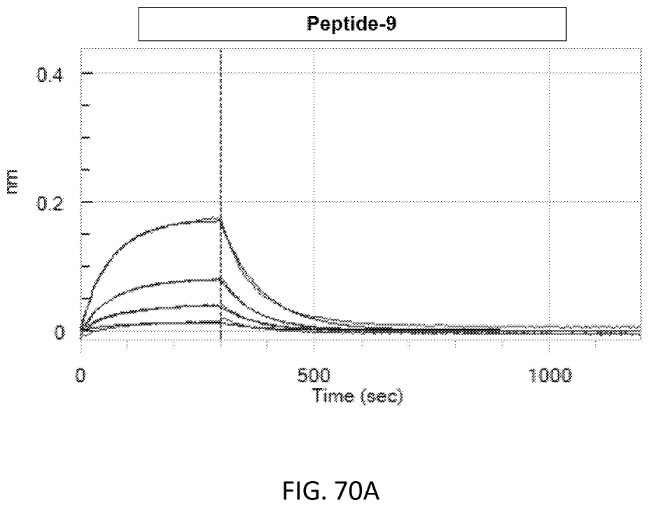

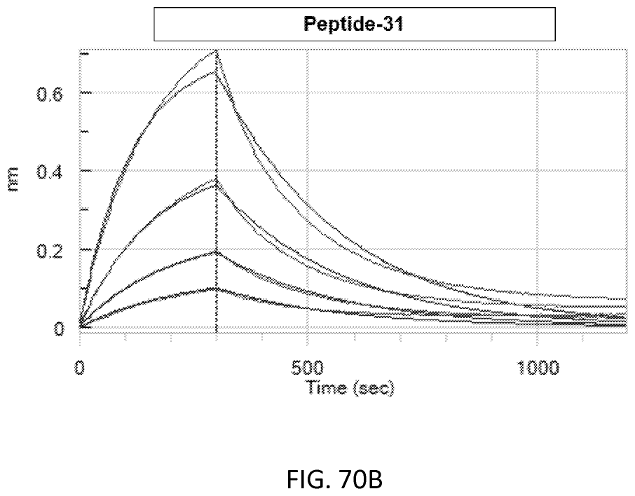

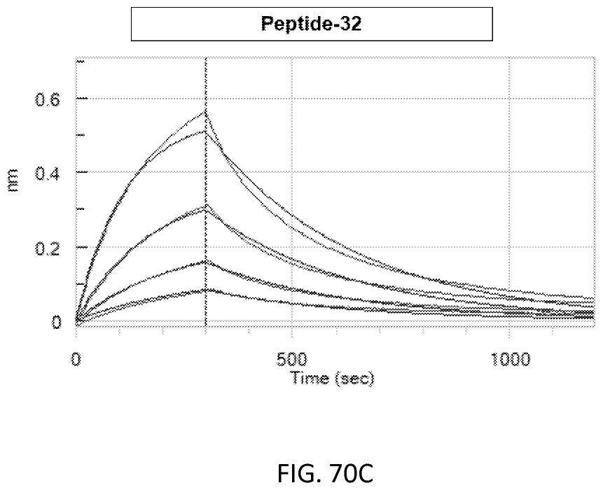

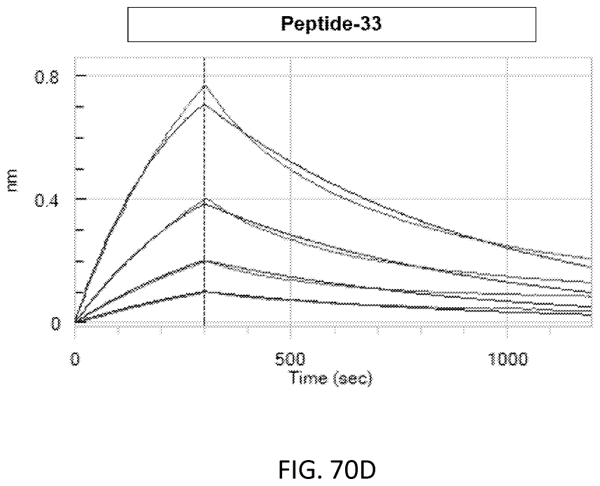

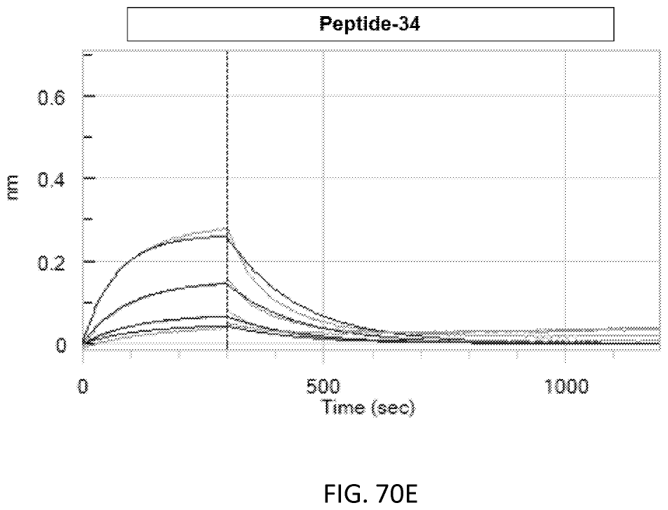

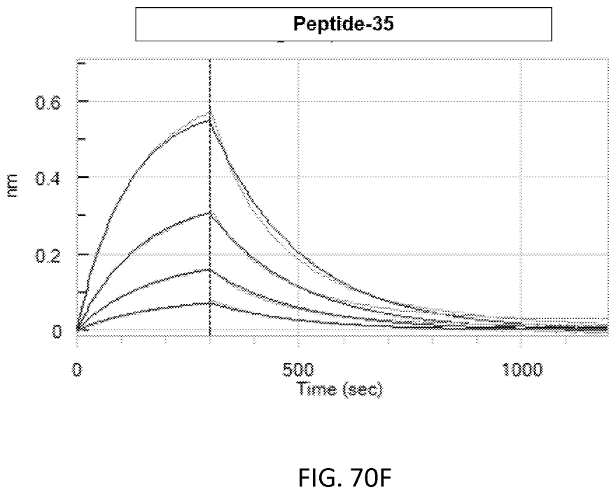

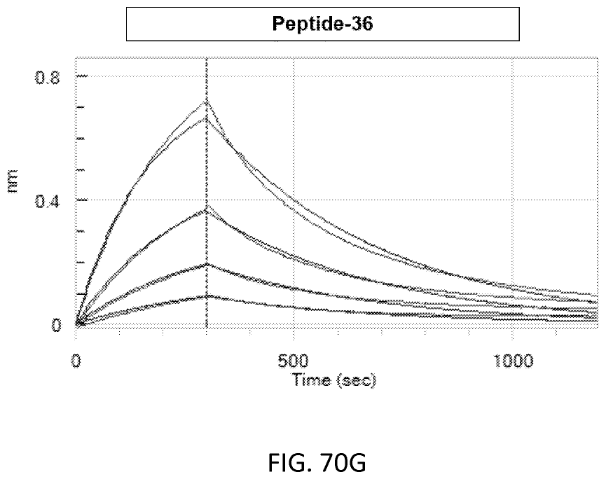

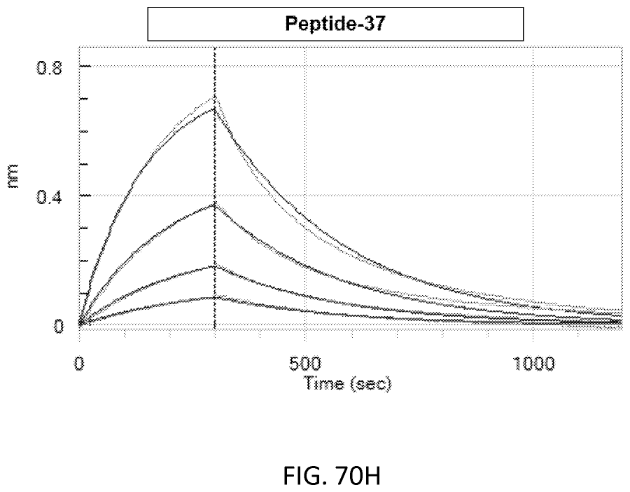

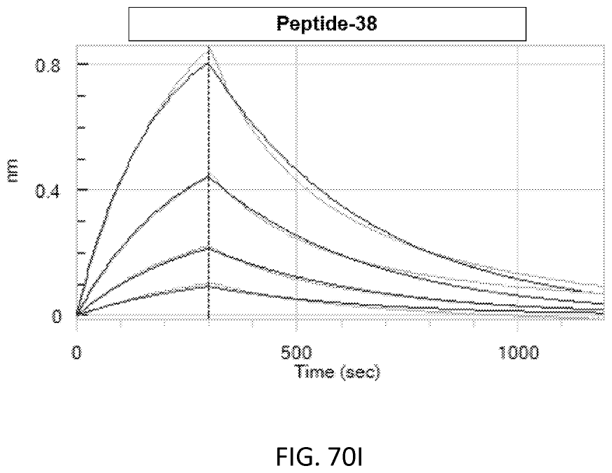

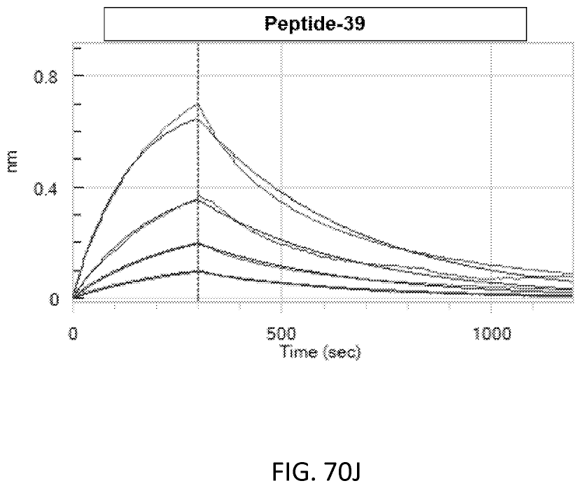

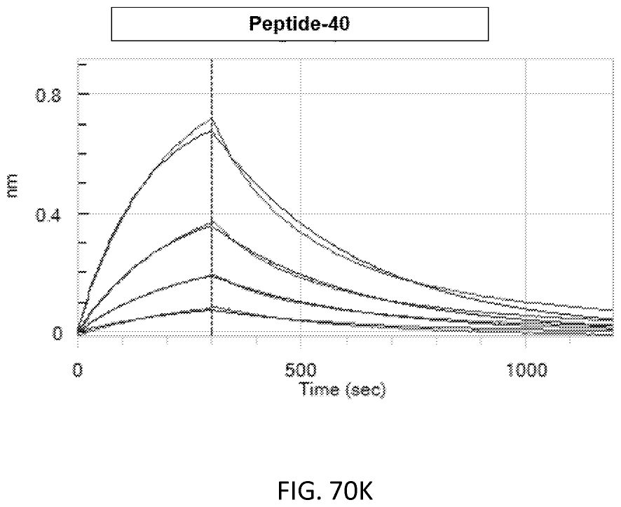

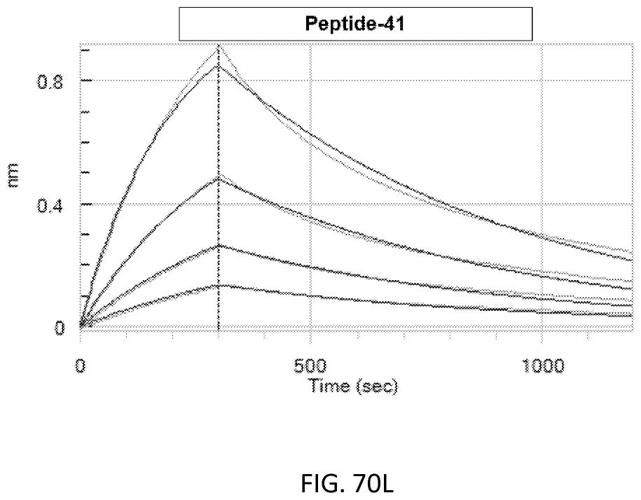

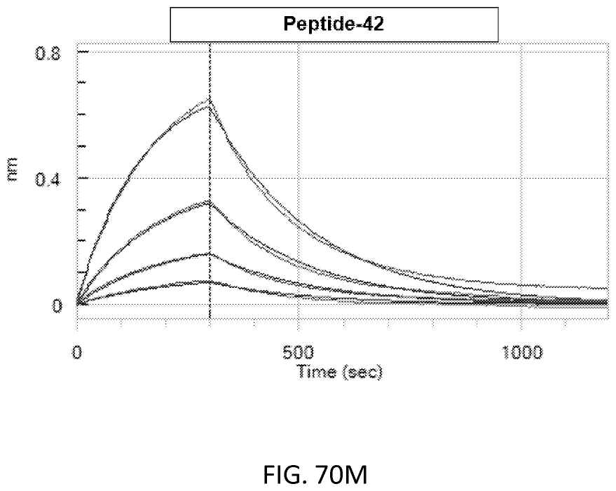

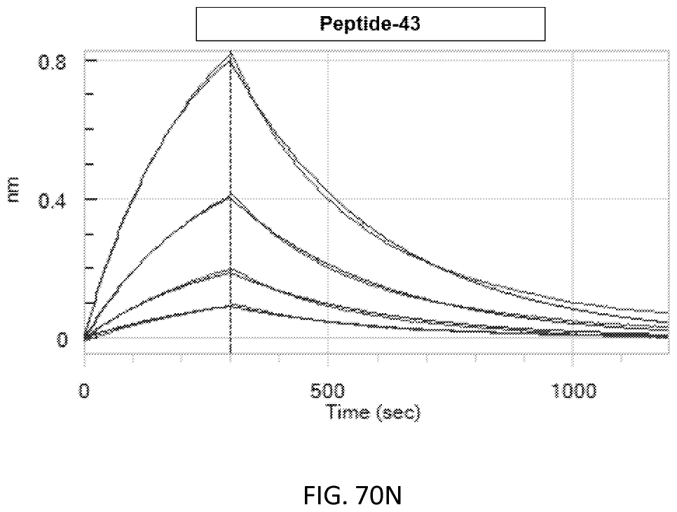

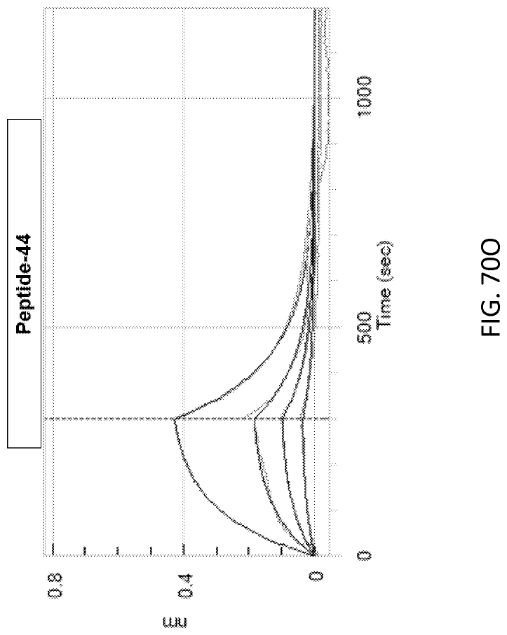

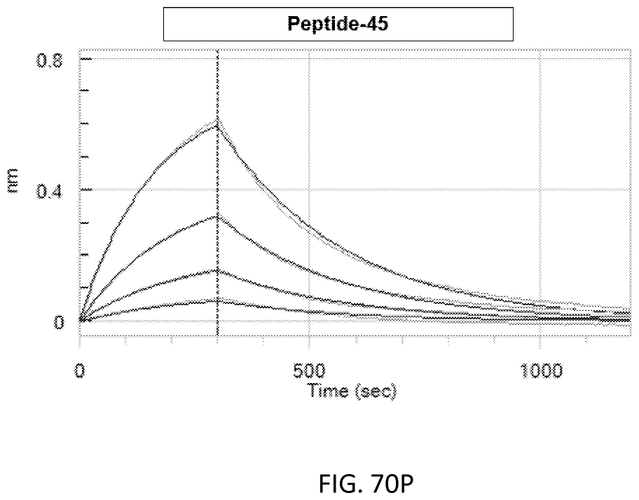

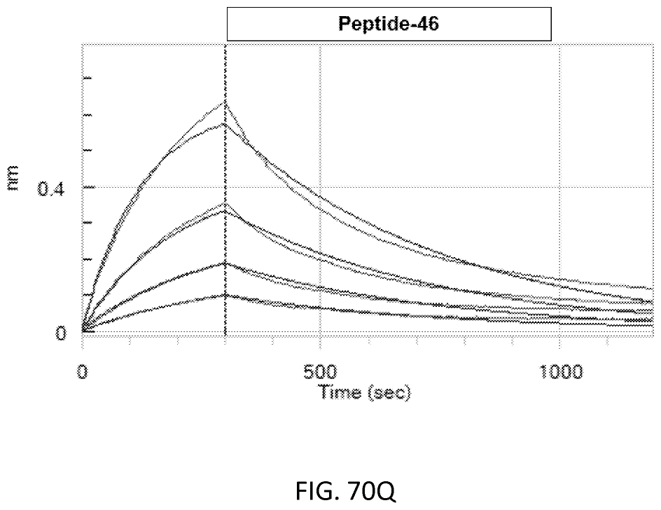

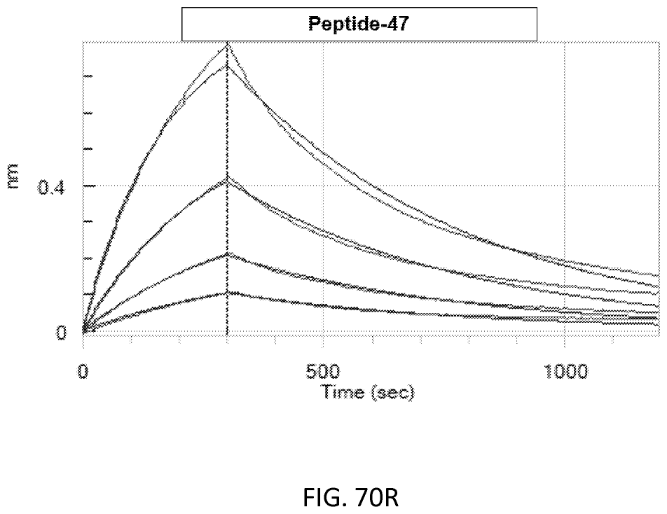

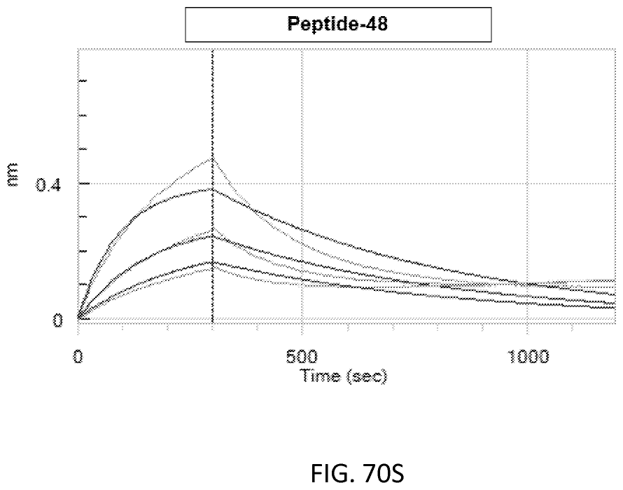

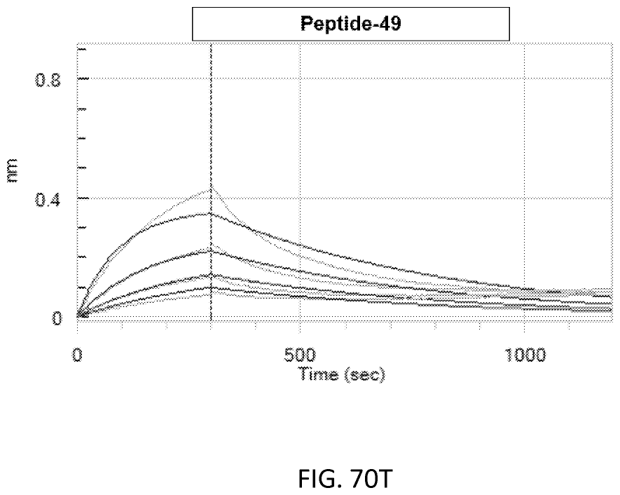

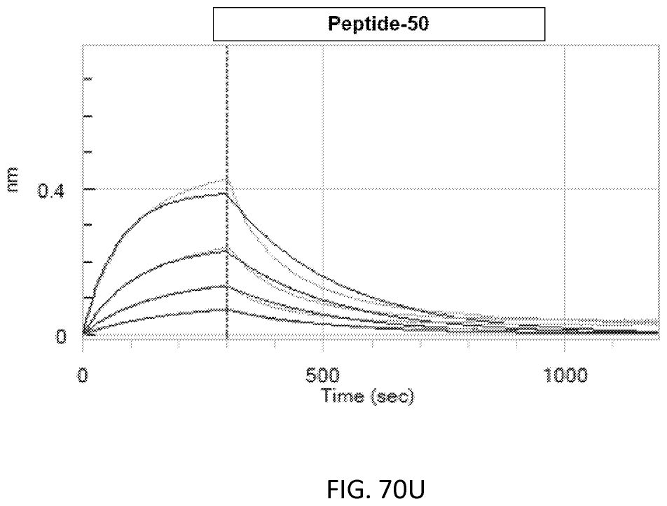

Kinetic binding of anti-CD28 scFv (SEQ ID NO: 9) to peptides were evaluated by bio-layer interferometry using an Octet RED96 instrument. Briefly, streptavidin biosensors were loaded with biotinylated peptides and baselined in buffer. Anti-CD28 scFv were titrated in solution at 100 nM, 50 nM, 25 nM, and 12.5 nM, then associated onto the peptide loaded sensors. After a short association period, sensors were transferred into buffer and the dissociation of bound anti-CD28 scFv was measured. The timing and steps of the experiment are shown in the accompanying table. Association and dissociation signals were recorded in real time and analyzed using a 1:1 binding model within the instrument software. Analysis using a 1:1 binding model enabled the calculation of the on and off rate constants as well as affinity, KD. FIGS. 70A-70F illustrate kinetic binding of anti-CD28 scFv binding to peptides as measured by Octet. FIGS. 70G-70U illustrate kinetic binding of peptides to the anti-CD28 scFv as measured by Octet. Kinetic sensorgrams are summarized in Tables 35-36.

TABLE 35
Timing and Steps of Assay

| Step | Time | |
|---|---|---|
| Baseline: Buffer | 60 | sec |
| Load: | 300 | sec |
| 200 nM Peptide | | |
| Baseline: Buffer | 300 | sec |
| Association in octet buffer | 300 | sec |
| 100 nM CD28 scFv | | |
| 50 nM CD28 scFv | | |
| 25 nM CD28 scFv | | |
| 12.5 nM CD28 scFv | | |
| Dissociation: Buffer | 900 | sec |

TABLE 36
Summary of Kinetic Data

| | Loading Sample | | | |
|---|---|---|---|---|
| Sample ID | ID | KD (M) | kon(1/Ms) | kdis(1/s) |
| Anti-CD28 | Peptide-31 | 7.18E−08 | 5.14E+04 | 3.69E−03 |
| scFv | | | | |
| Anti-CD28 | Peptide-32 | 4.99E−08 | 5.91E+04 | 2.95E−03 |
| scFv | | | | |
| Anti-CD28 | Peptide-33 | 5.28E−08 | 2.90E+04 | 1.53E−03 |
| scFv | | | | |
| Anti-CD28 | Peptide-34 | 8.88E−08 | 7.70E+04 | 6.84E−03 |
| scFv | | | | |
| Peptide-35 | Peptide-35 | 1.27E−07 | 3.90E+04 | 4.97E−03 |
| Anti-CD28 | Peptide-36 | 6.87E−08 | 3.67E+04 | 2.52E−03 |
| scFv | | | | |
| Anti-CD28 | Peptide-37 | 9.78E−08 | 3.59E+04 | 3.51E−03 |
| scFv | | | | |
| Anti-CD28 | Peptide-38 | 9.23E−08 | 3.00E+04 | 2.77E−03 |
| scFv | | | | |
| Anti-CD28 | Peptide-39 | 5.19E−08 | 5.11E+04 | 2.65E−03 |
| scFv | | | | |
| Anti-CD28 | Peptide-40 | 7.99E−08 | 3.88E+04 | 3.10E−03 |
| scFv | | | | |
| Anti-CD28 | Peptide-41 | 4.23E−08 | 3.64E+04 | 1.54E−03 |
| scFv | | | | |
| Anti-CD28 | Peptide-42 | 1.52E−07 | 2.84E+04 | 4.33E−03 |
| scFv | | | | |
| Anti-CD28 | Peptide-43 | 1.92E−07 | 1.67E+04 | 3.20E−03 |
| scFv | | | | |
| Anti-CD28 | Peptide-44 | 3.74E−07 | 2.17E+04 | 8.12E−03 |
| scFv | | | | |
| Anti-CD28 | Peptide-45 | 1.34E−07 | 2.71E+04 | 3.64E−03 |
| scFv | | | | |
| Anti-CD28 | Peptide-46 | 3.52E−08 | 6.19E+04 | 2.18E−03 |
| scFv | | | | |
| Anti-CD28 | Peptide-47 | 5.18E−08 | 3.89E+04 | 2.01E−03 |
| scFv | | | | |
| Anti-CD28 | Peptide-48 | 2.11E−08 | 8.89E+04 | 1.87E−03 |
| scFv | | | | |
| Anti-CD28 | Peptide-49 | 2.11E−08 | 8.82E+04 | 1.86E−03 |
| scFv | | | | |
| Anti-CD28 | Peptide-50 | 4.80E−08 | 9.23E+04 | 4.43E−03 |
| scFv | | | | |
| Anti-CD28 | Peptide-9 | 2.78E−07 | 4.15E+04 | 1.15E−02 |
| scFv | | | | |

### Example 22: PSMA Fab Binding to Peptides

### Equilibrium Binding via ELISA

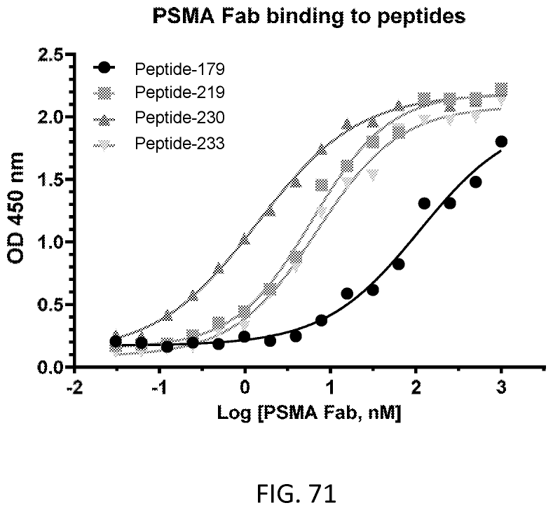

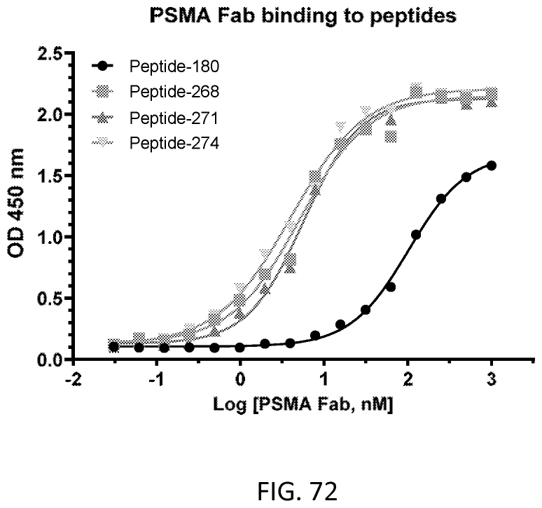

Peptides of Table 9 were evaluated for their ability to bind to PSMA Fab in a standard enzyme linked immunosorbent assay (ELISA) format. Specifically, peptides were evaluated for their ability to bind polypeptide sequence PSMA Fab (SEQ ID NOs: 19, 20). Briefly, biotinylated peptides were captured on neutravidin coated plates. PSMA Fab diluted in buffer was then added to the peptide captured plates. Bound antibody was detected using a standard horse radish peroxidase conjugate secondary antibody. The concentration of PSMA Fab required to achieve 50% maximal signal (EC₅₀) was calculated using Graphpad Prism software. Exemplary binding curves for PSMA Fab binding to peptides are shown in FIGS. 71-72. EC₅₀ₛ for PSMA Fab binding to the peptides are shown in Tables 37-39.

### Binding Inhibition by Peptide Masks

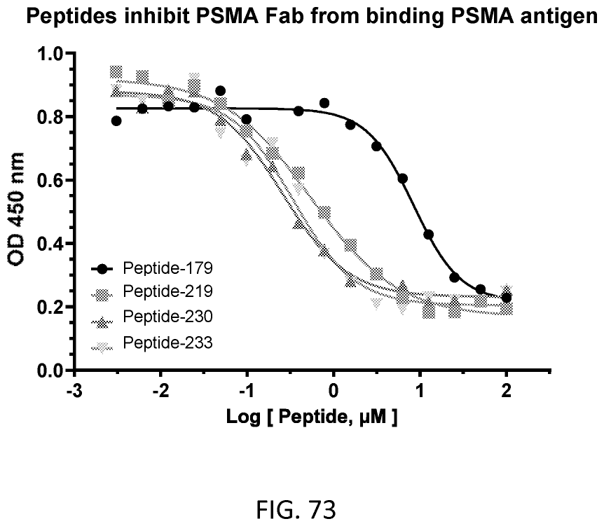

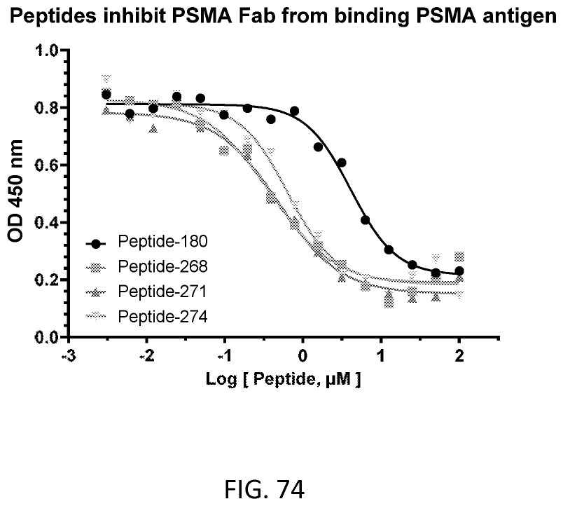

The peptides of Table 9 were further evaluated for their ability to inhibit PSMA Fab (SEQ ID NOs: 19, 20) from binding to the PSMA antigen in a standard ELISA format. Briefly, biotinylated PSMA antigen was captured on neutravidin coated plates. PSMA Fab fixed at 0.3 nanomolar (nM) concentration was pre-incubated with 0-100 micromolar (μM) titrated peptides. After a short pre-incubation period, the mixture of titrated peptide with fixed PSMA Fab were added to the PSMA antigen coated plates. After a short incubation on the plates, bound PSMA Fab was detected with a standard horse radish peroxidase conjugated secondary antibody. The concentration of peptide required to reduce the maximum signal by 50% (IC₅₀) was calculated in Graphpad Prism software. FIGS. 73-74 illustrate inhibition of PSMA Fab binding to PSMA by the peptide masks as measured by ELISA. IC₅₀ₛ are provided in Tables 37-39.

### Kinetic Binding via BLI

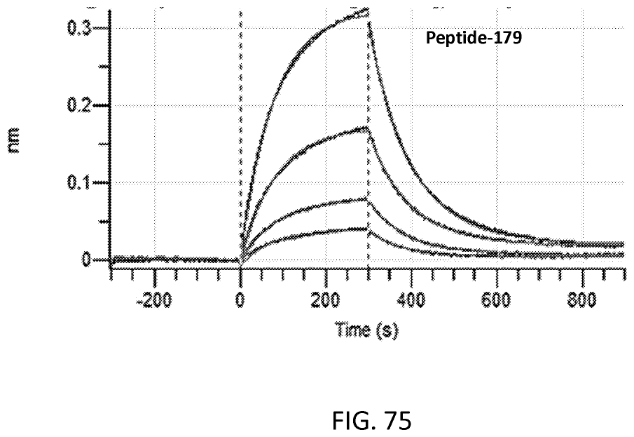

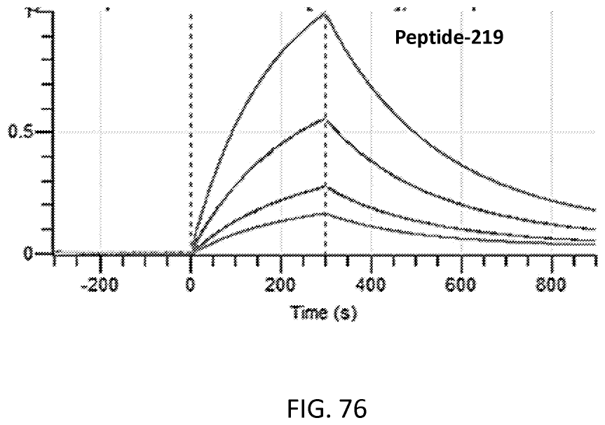

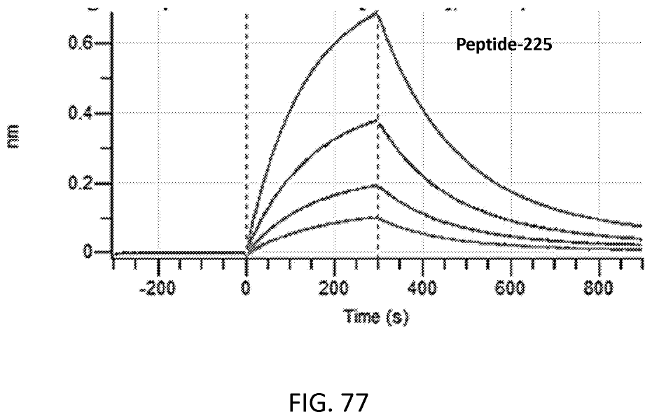

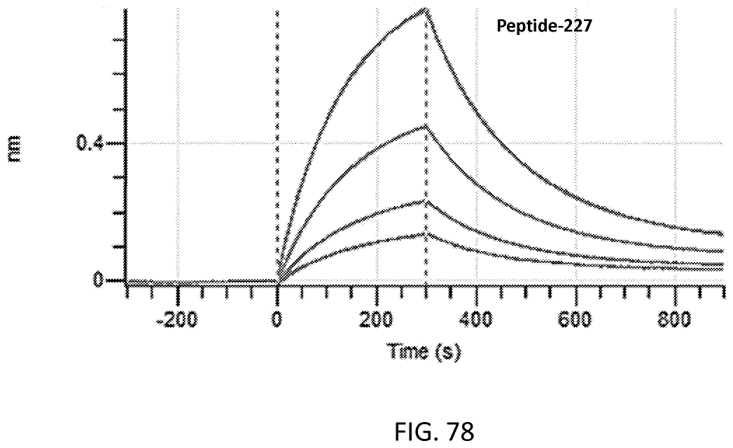

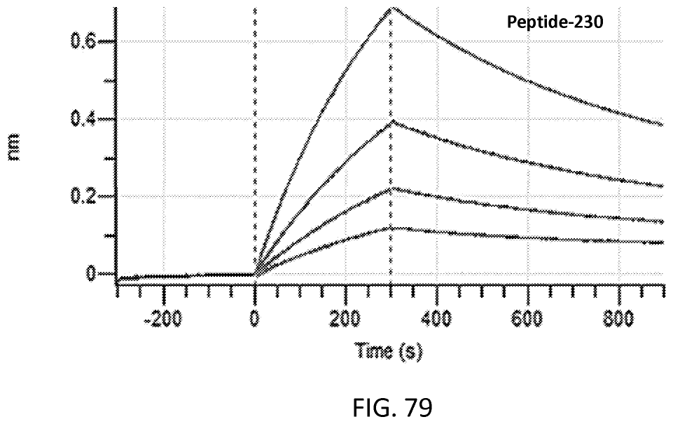

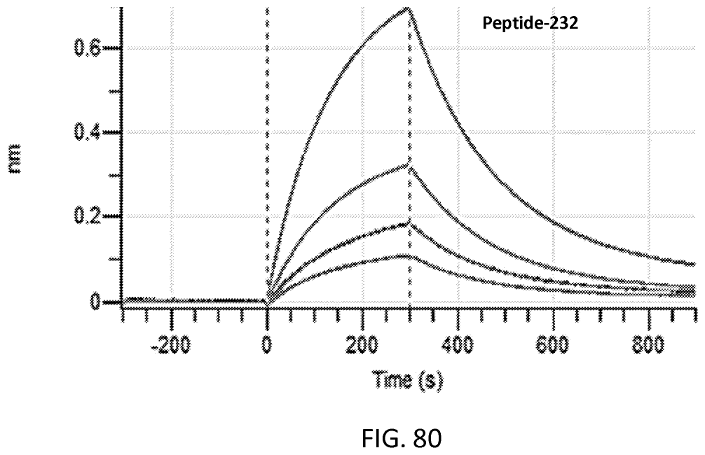

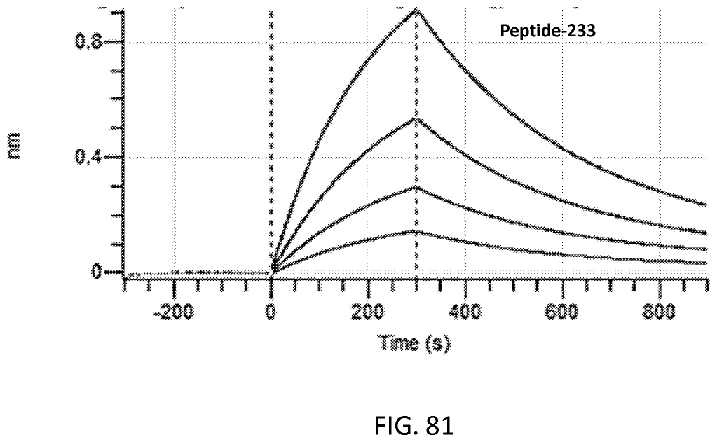

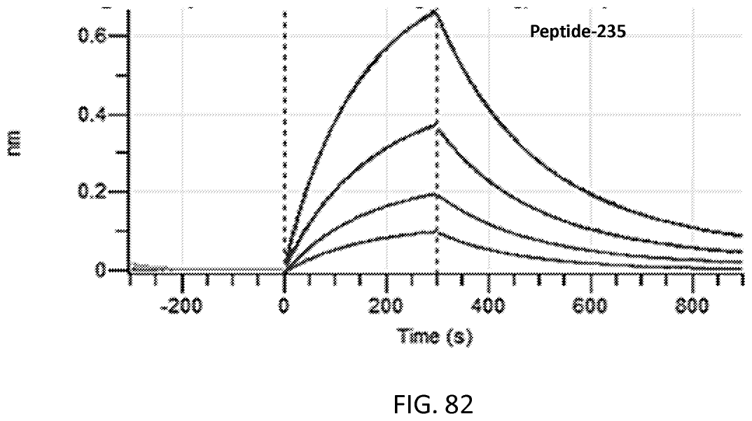

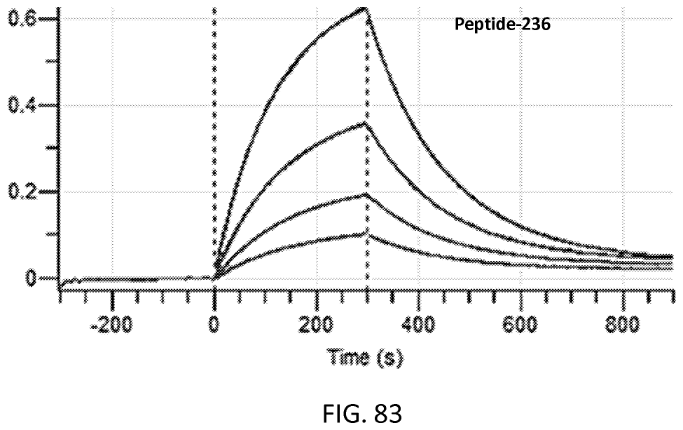

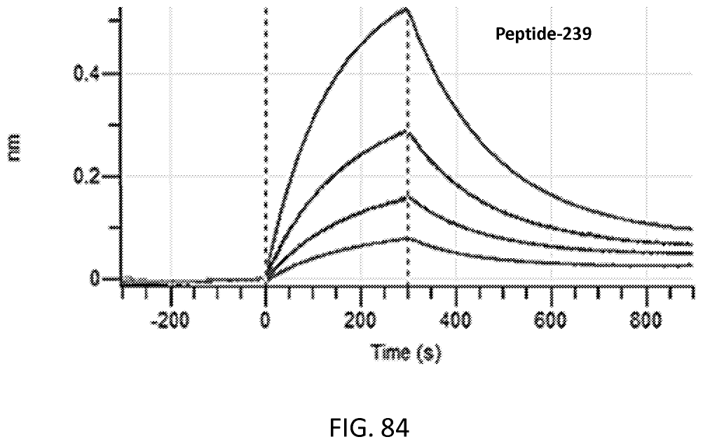

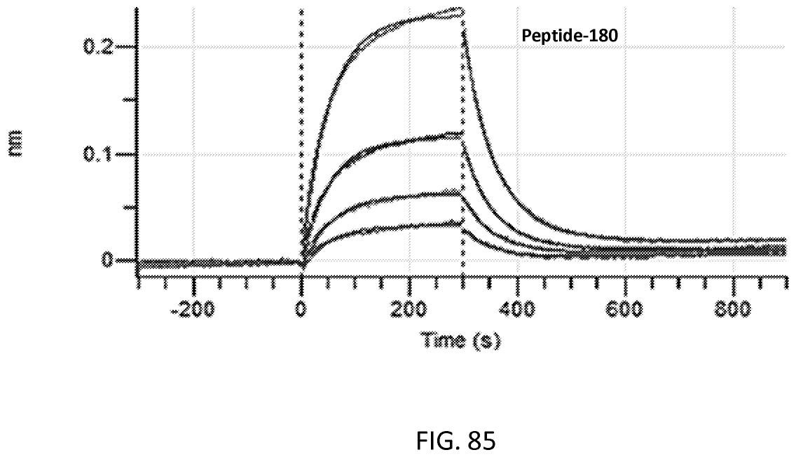

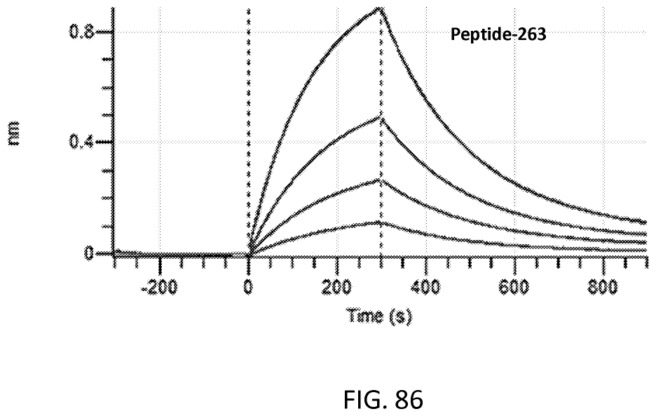

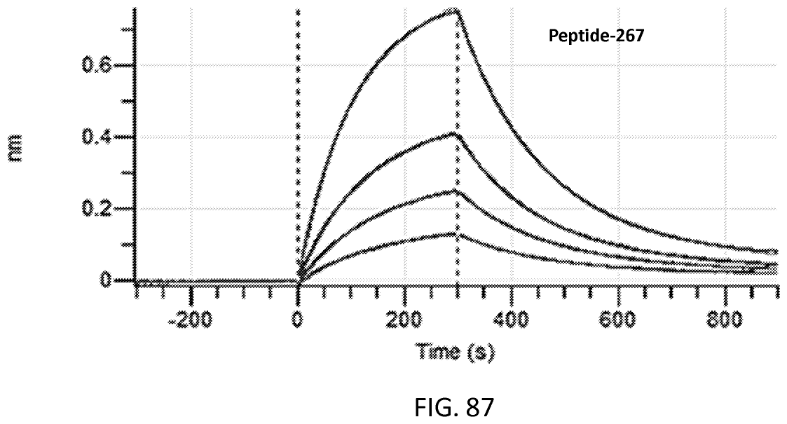

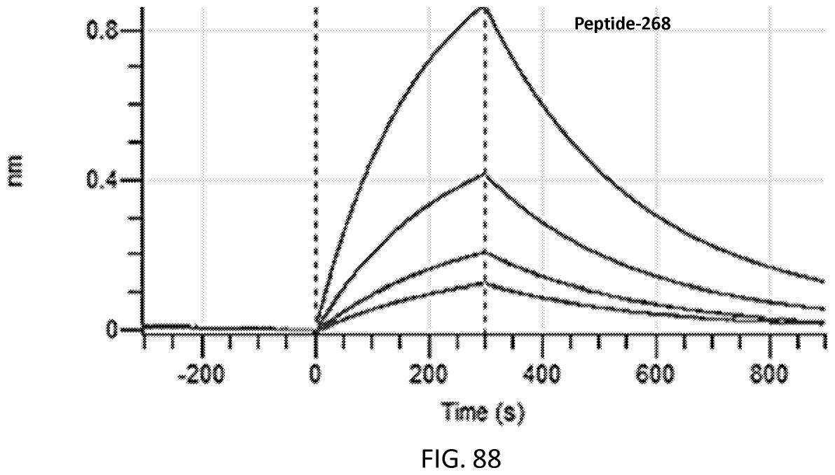

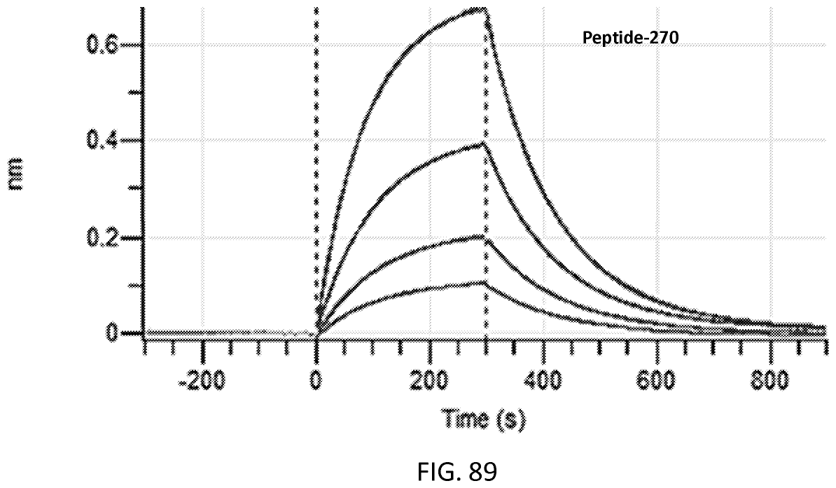

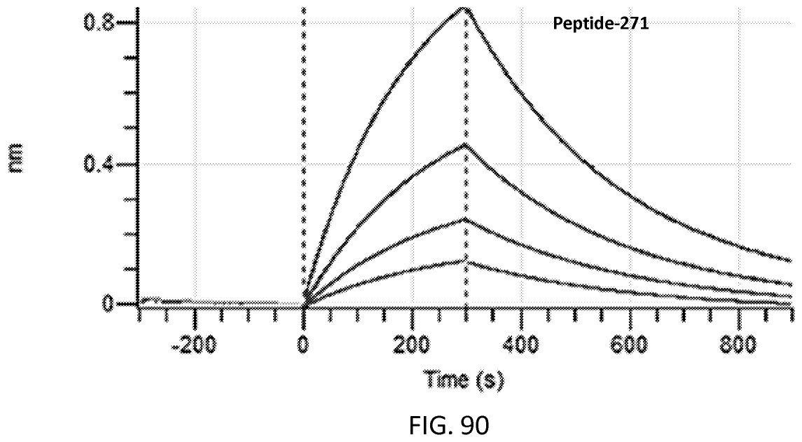

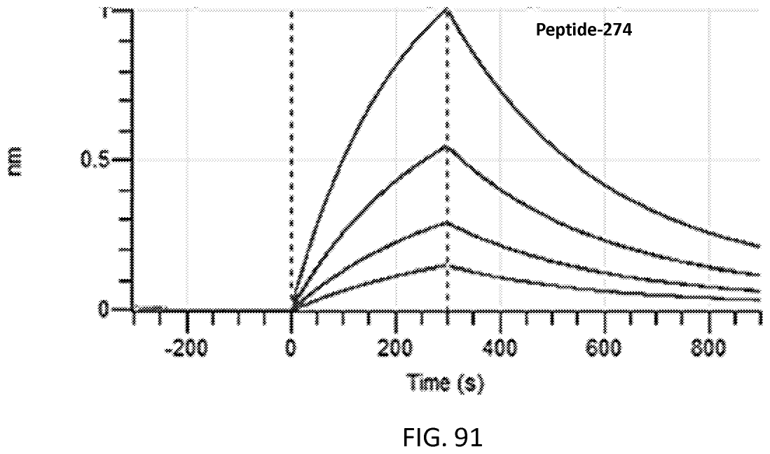

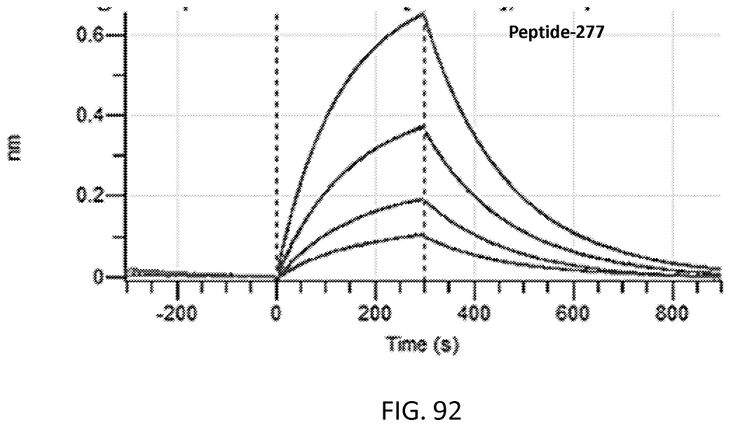

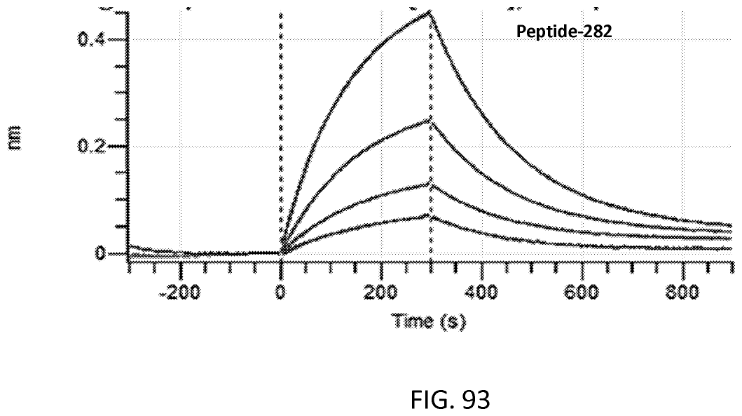

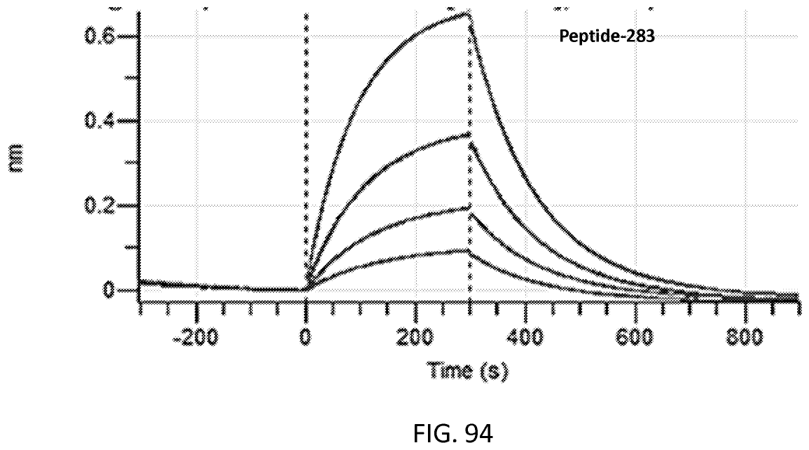

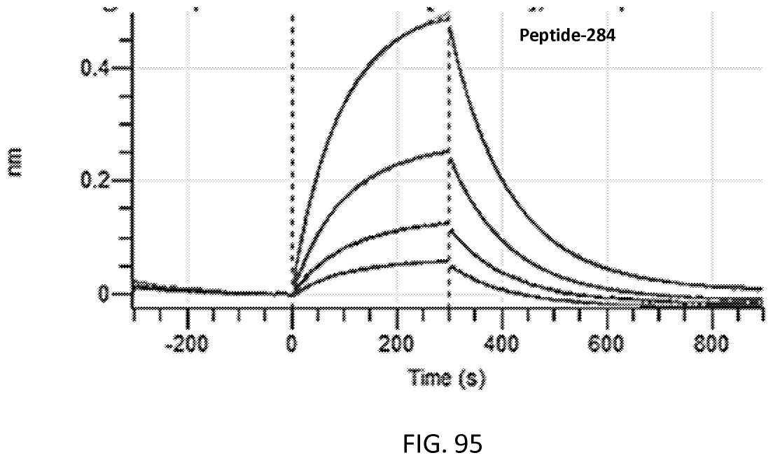

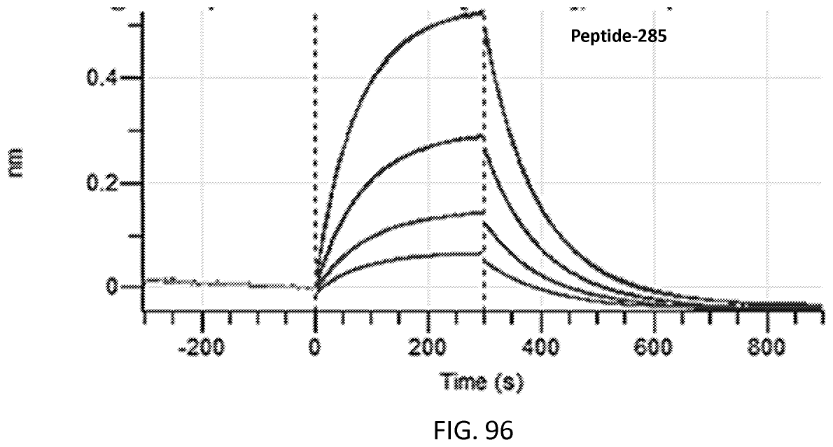

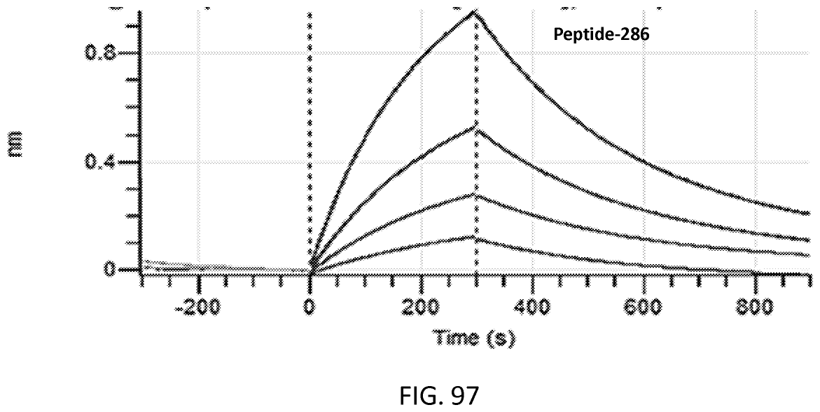

Kinetic binding of the peptides of Table 9 to PSMA Fab (SEQ ID NOs: 19, 20) was evaluated using biolayer interferometry (BLI). Briefly, biotinylated peptides were loaded onto a streptavidin coated Octet® V SAX biosensor, quenched in biocytin, and baselined in buffer. PSMA Fab was titrated in solution (100 nM, 50 nM, 25 nM, and 12.5 nM) and associated onto the peptide loaded sensor. After a short association period, sensors were transferred into buffer and the dissociation of bound Ab-1 was measured. Association and dissociation signals were recorded in real time and analyzed using a 1:1 binding model within the instrument software. Analysis using a 1:1 binding model enabled the calculation of the on and off rate constant as well as affinity, Kn. Binding sensorgrams are shown in FIGS. 75-97. Half-life (t_{1/2}) values obtained from the kinetic binding measurements are provided in Tables 38-39.

### Peptide Mask Properties

Peptide off rate, binding affinity, and inhibition strength are key design features of peptide masks as described herein. Peptides with the lowest EC₅₀ value and lowest IC₅₀ value with fast off rates, lowest t_{1/2} values, are desired. Peptides with high binding affinity and inhibition capacity imply masking strength while a fast off rate ensures that the mask comes off the antibody once the antibody is cleaved by protease.

TABLE 37

| Peptide | Binding EC₅₀ (nM) | Inhibition (IC₅₀) (μM) |
|---|---|---|
| Peptide-157 | >1,000 | |
| Peptide-158 | high background | 17.32 |
| Peptide-159 | >1,000 | |
| Peptide-160 | >1,000 | |
| Peptide-161 | >1,000 | |
| Peptide-162 | >1,000 | |
| Peptide-163 | >1,000 | |
| Peptide-164 | high background | >100 |
| Peptide-165 | >1,000 | |
| Peptide-166 | >1,000 | 19.22 |
| Peptide-167 | >1,000 | >100 |
| Peptide-168 | >1,000 | |
| Peptide-169 | >1,000 | |
| Peptide-170 | >1,000 | |
| Peptide-171 | >1,000 | |
| Peptide-172 | >1,000 | |
| Peptide-173 | >1,000 | |
| Peptide-174 | >1,000 | |
| Peptide-175 | >1,000 | 17.83 |
| Peptide-176 | >1,000 | |
| Peptide-177 | >1,000 | |
| Peptide-178 | >1,000 | 20.78 |

TABLE 38

| | | Inhibition | Off rate |
|---|---|---|---|
| Peptide | Binding EC₅₀ (nM) | IC₅₀ (μM) | t_{1/2} (min) |
| Peptide-179 | 5.9 | 4.44 | <1 |
| Peptide-180 | 33 | 4.54 | <1 |
| Peptide-181 | 3 | 11.18 | |
| Peptide-182 | >3000 | >100 | |
| Peptide-183 | | >100 | |
| Peptide-184 | >3000 | >100 | |
| Peptide-185 | >3000 | 23.01 | |
| Peptide-186 | >3000 | >100 | |
| Peptide-187 | >3000 | >100 | |
| Peptide-188 | >3000 | 7.22 | |
| Peptide-189 | >3000 | >100 | |
| Peptide-190 | >3000 | >100 | |

TABLE 39

| | | Inhibition | Off rate |
|---|---|---|---|
| Peptide | Binding EC₅₀ (nM) | IC₅₀ (μM) | t_{1/2} (min) |
| Peptide-179 | 120.0 | 4.60 | 1.09 |
| Peptide-180 | | 4.50 | 0.75 |
| Peptide-181 | | 26.09 | |
| Peptide-191 | | >100 | |
| Peptide-192 | | >100 | |
| Peptide-193 | | >100 | |
| Peptide-194 | | 10.29 | <1 |
| Peptide-195 | | 19.26 | |
| Peptide-196 | | >100 | |
| Peptide-197 | | >100 | |
| Peptide-198 | | 18.85 | |
| Peptide-199 | | >100 | |
| Peptide-200 | | 12.18 | |
| Peptide-201 | | >100 | |
| Peptide-202 | | 10.95 | <1 |
| Peptide-203 | | 36.13 | |
| Peptide-204 | | 9.07 | <1 |
| Peptide-205 | | 5.52 | <1 |
| Peptide-206 | | 28.66 | |
| Peptide-207 | | 9.86 | |
| Peptide-208 | | >100 | |
| Peptide-209 | | 10.20 | |
| Peptide-210 | | >100 | |
| Peptide-211 | | >100 | |
| Peptide-212 | | 6.39 | |
| Peptide-213 | | >100 | |

### Example 23: Sequence Activity Relationships-PSMA Mask Peptides

Sequence activity relationships (SAR) were established for peptide-179 and peptide-180 which were found to exhibit strong binding to the PSMA Fab. The sequence activity relationships were established by mutating individual residues within each peptide to alanine and measuring the influence of each mutation on the ability of the peptide to inhibit the ability of the PSMA Fab from binding to PSMA by competition ELISA experiments as described above in Example 23. IC₅₀ₛ forbidding inhibition of the anine scanning sequences are provided in Tables 40-41. The tables also designate each alanine mutation as tolerant (T), somewhat tolerant (ST), or intolerant (IT) based on changes in binding inhibition ability relative to the non-mutated peptide. Cys-5 and Cys-11 of peptide-179 and peptide-180 were not mutated.

TABLE 40
Competition Binding ELISA - Alanine Scanning Sequences of Peptide-179

| | Amino Acid Sequence | SEQ ID | Mutation | Inhibition |
|---|---|---|---|---|
| Peptide | (N to C) | NO: | Status | IC₅₀ (uM) |
| Peptide-179 | LMKWCTPRWMCAAE | 2025 | — | 6.49 |
| Peptide-241 | AMKWCTPRWMCAAE | 2087 | T | 9.16 |
| Peptide-242 | LAKWCTPRWMCAAE | 2088 | T | 13.74 |
| Peptide-2687 | LMAWCTPRWMCAAE | 2089 | ST | 40.75 |
| Peptide-244 | LMKACTPRWMCAAE | 2090 | ST | 34.51 |
| Peptide-245 | LMKWCAPRWMCAAE | 2091 | ST | 49.73 |
| Peptide-246 | LMKWCTARWMCAAE | 2092 | IT | >100 |
| Peptide-247 | LMKWCTPAWMCAAE | 2093 | T | 19.73 |
| Peptide-248 | LMKWCTPRAMCAAE | 2094 | IT | >100 |
| Peptide-249 | LMKWCTPRWACAAE | 2095 | IT | >100 |
| Peptide-250 | LMKWCTPRWMCAAA | 2096 | T | 6.64 |

TABLE 41
Competition Binding ELISA - Alanine Scanning Sequences of Peptide-180

| | Amino Acid Sequence | SEQ ID | Mutation | Inhibition |
|---|---|---|---|---|
| Peptide | (N to C) | NO: | Status | (IC₅₀) (uM) |
| Peptide-180 | IAVLCQPNWMCAGF | 2026 | — | 5.23 |
| Peptide-251 | AAVLCQPNWMCAGF | 2097 | T | 8.45 |
| Peptide-252 | IAALCQPNWMCAGF | 2098 | T | 5.696 |
| Peptide-253 | IAVACQPNWMCAGF | 2099 | ST | 28.22 |
| Peptide-254 | IAVLCAPNWMCAGF | 2100 | T | 15.66 |
| Peptide-255 | IAVLCQANWMCAGF | 2101 | IT | >100 |
| Peptide-256 | IAVLCQPAWMCAGF | 2102 | T | 2.16 |
| Peptide-257 | LAVLCQPNAMCAGF | 2103 | IT | >100 |
| Peptide-258 | IAVLCQPNWACAGF | 2104 | IT | >100 |
| Peptide-259 | IAVLCOPNWMCAAF | 2105 | ST | 67.90 |
| Peptide-260 | IAVLCQPNWMCAGA | 2106 | ST | 59.95 |

### Example 24. Optimization Phage Library Construction of Peptide-180

Using the peptide sequence activity relationships of Example 24, DNA oligo libraries were constructed where codons encoding critical residues within peptide-180 were minimally mutated, and codons encoding non-critical residues that were tolerated in the alanine scanning mutation studies (e.g., IIe-1, Ala-2, Val-3, Gln-6, Asn-8, and Ala-12) were heavily mutated. The resulting oligos were cloned into bacteriophage vectors used to display the SAR guided peptides via fusion to the pIII filament of the bacteriophage. The relevant vectors were then used to produce the phage optimization libraries via amplification in bacteria using standard techniques in the field.

### Example 25: Panning of the Optimized Phage Library of Peptide-180

Once the phage optimization libraries were completed, phage libraries were bio-panned using PSMA Fab loaded beads. Multiple rounds of panning were performed where bacteriophage was allowed to bind to PSMA Fab loaded beads. washed. eluted, and amplified. After panning, phage infected bacteria were plated out and colonies picked into 96 well blocks. Clonal phage was then amplified and separated from bacterial cells via centrifugation. Phage containing supemnatants were tested in binding ELISAs against PSMA Fab coated plates in the presence or absence of saturating concentrations of PSMA antigen. Phage able to bind PSMA Fab were selected for sequence analysis if the binding signal was reduced in the presence of PSMA. SAR guided optimization phage library-panning results of peptide-180 are shown in Table 42.

TABLE 42
SAR guided optimization phage library of peptide-180.

| | | Inhibition | Off rate |
|---|---|---|---|
| Peptide | Binding EC₅₀ (nM) | IC₅₀ (μM) | t_{1/2} (min) |
| Peptide-180 | 102.4 | 4.14 | 0.6 |
| Peptide-261 | | 1.30 | |
| Peptide-262 | | 1.30 | |
| Peptide-263 | 8.4 | 0.62 | 2.3 |
| Peptide-264 | | 1.14 | |
| Peptide-265 | | 1.78 | |
| Peptide-266 | | 1.81 | |
| Peptide-267 | 4.8 | 0.91 | 1.9 |
| Peptide-268 | 5.1 | 0.25 | 3.0 |
| Peptide-269 | | 9.14 | |
| Peptide-270 | 17.3 | 0.70 | 1.4 |
| Peptide-271 | 5.7 | 0.30 | 3.3 |
| Peptide-272 | | 1.92 | |
| Peptide-273 | | 0.90 | |
| Peptide-274 | 4.0 | 0.33 | 3.3 |
| Peptide-275 | | >100 | |
| Peptide-276 | | 3.46 | |
| Peptide-277 | 4.1 | 0.15 | 1.9 |
| Peptide-278 | | 2.40 | |
| Peptide-279 | | 5.18 | |
| Peptide-280 | | 6.65 | |
| Peptide-281 | | 1.02 | |
| Peptide-282 | 9.2 | 0.19 | 1.9 |
| Peptide-283 | 7.8 | 0.82 | 1.4 |
| Peptide-284 | 20.0 | 0.97 | 1.4 |
| Peptide-285 | 25.0 | 0.71 | 1.1 |
| Peptide-286 | 4.0 | 0.64 | 3.3 |
| Peptide-287 | | 2.22 | |

### Example 26: Panning of the Optimized Phage Library

Clonal phage were harvested as crude supernatants and screened via standard ELISAs. Briefly, biotinylated PSMA Fab was captured on neutravidin coated plates. Prior to the addition of clonal phage. wells were incubated with blocking buffer and PSMA antigen or blocking buffer alone. Without washing or aspirating, clonal phage supernatants were then added to the wells and incubated for a short time. Wells were then washed followed by detection of bound phage using a horse radish conjugated anti-M13 antibody. Clonal phage of interest were then sent for sequence analysis.

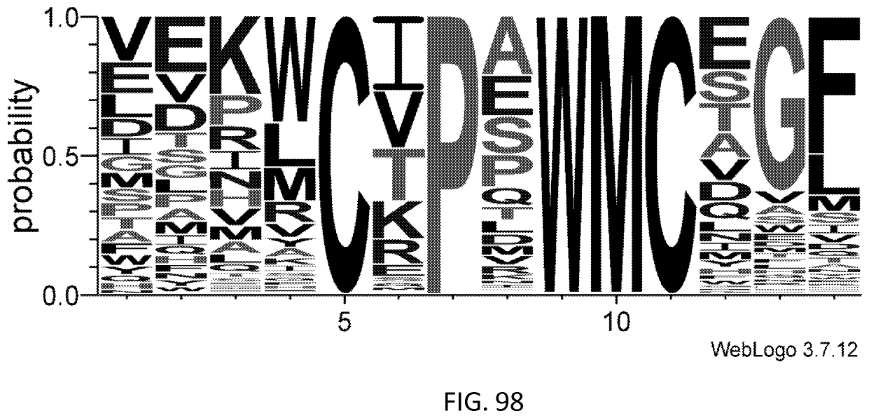

Phage panning results of peptide-180 library sequences are shown in Table 10. 2323 clonal phage sequences were identified. A consensus sequence was calculated from all the sequences of Table 10 using WebLogo 3.7.12. The consensus sequence is shown in FIG. 98.

### Example 27. Optimization Phage Library Construction of Peptide-179

Using the peptide sequence activity relationships of Example B, DNA oligo libraries were constructed where codons encoding critical residues within peptide-179 were minimally mutated, and codons encoding non-critical residues that were tolerated in the alanine scanning mutation studies (e.g., Leu-1, Met-2, Arg-8, Ala-12, Ala-13, and Glu-14) were heavily mutated. The resulting oligos were cloned into bacteriophage vectors used to display the SAR guided peptides via fusion to the pill filament of the bacteriophage. The relevant vectors were then used to produce the phage optimization libraries via amplification in bacteria using standard techniques in the field.

### Example 28: Panning of the optimized Phage library of Peptide-179

Once the phage optimization libraries were completed, phage libraries were bio-panned using PSMA Fab loaded beads. Multiple rounds of panning were performed where bacteriophage was allowed to bind to PSMA Fab loaded beads, washed, eluted, and amplified. After panning, phage infected bacteria were plated out and colonies picked into 96 well blocks. Clonal phage was then amplified and separated from bacterial cells via centrifugation. Phage containing supernatants were tested in binding ELISAs against PSMA Fab coated plates in the presence or absence of saturating concentrations of PSMA antigen. Phage able to bind PSMA Fab were selected for sequence analysis if the binding signal was reduced in the presence of PSMA. SAR guided optimization phage library panning results of peptide-179 are shown in Table 43.

TABLE 43
SAR guided optimization phage library of peptide-179.

| | | Inhibition | Off rate |
|---|---|---|---|
| Peptide | Binding EC₅₀ (nM) | IC₅₀ (μM) | t_{1/2} (min) |
| Peptide-179 | 109.6 | 7.71 | 1.2 |
| Peptide-214 | | 3.54 | |
| Peptide-215 | | 2.94 | |
| Peptide-216 | | 2.91 | |
| Peptide-217 | | 2.49 | |
| Peptide-218 | | 2.85 | |
| Peptide-219 | 6.1 | 0.49 | 2.9 |
| Peptide-220 | | 1.57 | |
| Peptide-221 | | 2.13 | |
| Peptide-222 | | 4.16 | |
| Peptide-223 | | 1.47 | |
| Peptide-224 | | 1.88 | |
| Peptide-225 | 9.7 | 0.34 | 2.1 |
| Peptide-226 | | 2.68 | |
| Peptide-227 | 7.7 | 0.76 | 2.1 |
| Peptide-228 | | 1.92 | |
| Peptide-229 | | 4.07 | |
| Peptide-230 | 1.4 | 0.26 | 6.4 |
| Peptide-231 | | 1.27 | |
| Peptide-232 | 7.2 | 0.96 | 2.1 |
| Peptide-233 | 6.9 | 0.36 | 3.9 |
| Peptide-234 | | 2.08 | |
| Peptide-235 | 10.5 | 0.32 | 2.3 |
| Peptide-236 | 19.8 | 0.38 | 1.8 |
| Peptide-237 | | 2.70 | |
| Peptide-238 | | 1.55 | |
| Peptide-239 | 9.4 | 0.62 | 2.0 |
| Peptide-240 | | >100 | |

### Example 29: Polypeptide Complex Binding to PSMA

Polypeptide complexes were evaluated for their ability to bind PSMA as in a standard enzyme linked immunosorbent assay (ELISA) format. Polypeptide complex binding of PSMA was measured before and after protease treatment where indicated. Briefly, biotinylated antigen was captured on neutravidin coated plates. Polypeptide complex molecules were treated with active-matrix metalloproteinase-9 (MMP9) where indicated. Polypeptide complex molecules diluted in buffer were then added to the antigen coated plates. Bound polypeptide complex was detected using a standard horse radish peroxidase conjugate secondary antibody. The concentration of polypeptide complex required to achieve 50% maximal signal (EC₅₀) was calculated in Graphpad Prism.

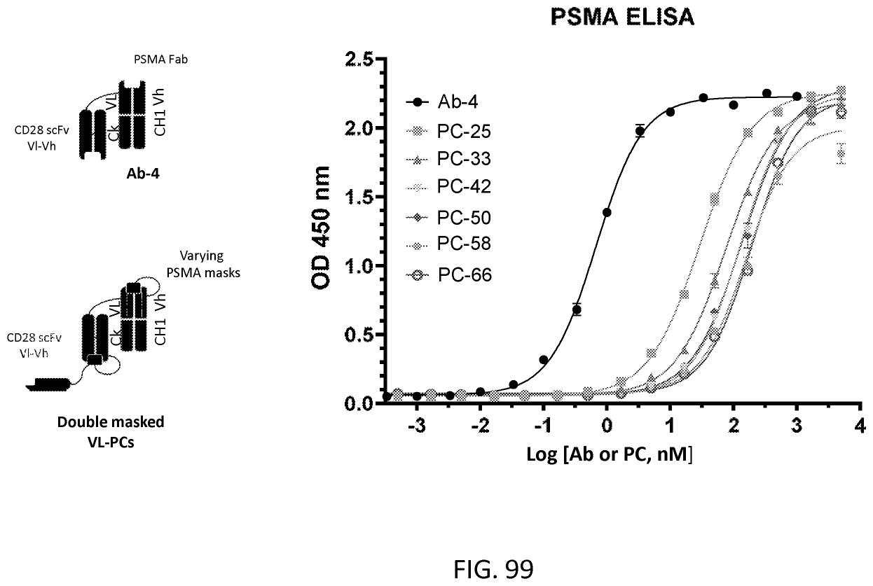

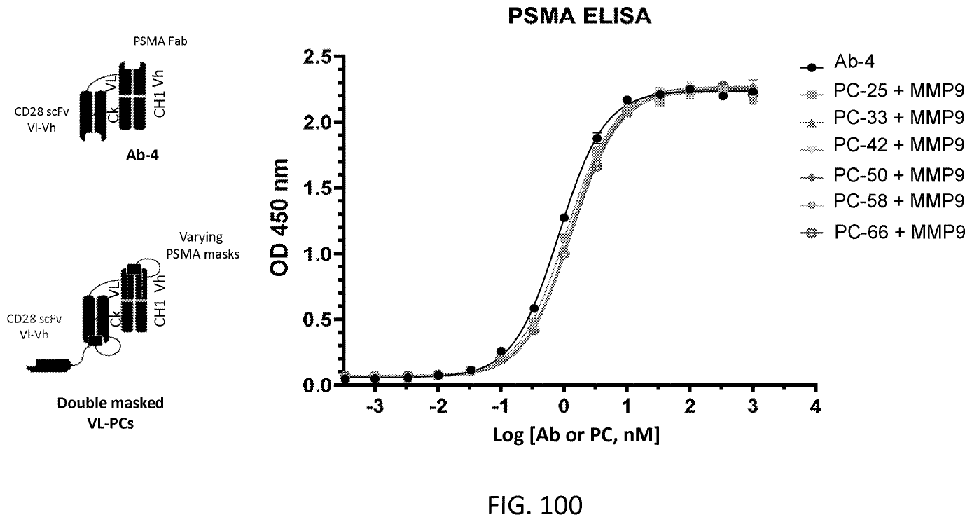

Binding of the polypeptide complexes of the disclosure to PSMA is shown in FIG. 99. Binding of polypeptide complexes of the disclosure to PSMA following treatment with matrix metalloproteinase 9 (MMP9) is shown in FIG. 100. Table 44 summarizes EC₅₀ values for the binding of polypeptide complexes to PSMA, and to PSMA following MMP9 treatment. The EC₅₀ shift was used to calculate the PSMA masking efficiency. Peptide-274 showed the strongest mask efficiency.

TABLE 44

| Polypeptide | | PSMA EC₅₀ | Cleaved PC, | EC50 |
|---|---|---|---|---|
| Complex | PSMA Mask | (nM) | PSMA EC₅₀ | shift |
| Ab-4 | — | 0.7 | 0.8 | — |
| PC-25 | Peptide-219 | 29.6 | 1.1 | 44x |
| PC-33 | Peptide-230 | 76.3 | 1.2 | 113x |
| PC-42 | Peptide-233 | 129.1 | 1.3 | 192x |
| PC-50 | Peptide-268 | 138.5 | 1.3 | 205x |
| PC-58 | Peptide-271 | 155.1 | 1.2 | 230x |
| PC-66 | Peptide-274 | 193.9 | 1.3 | 288x |

### Example 30: Polypeptide complex binding to CD28

Polypeptide complexes were evaluated for their ability to bind CD28 as in a standard enzyme linked immunosorbent assay (ELISA) format. Polypeptide complex binding of CD28 was measured before and after protease treatment where indicated. Briefly, biotinylated antigen was captured on neutravidin coated plates. Polypeptide complex molecules were treated with active-matrix metalloproteinase-9 (MMP9) where indicated. Polypeptide complex molecules diluted in buffer were then added to the antigen coated plates. Bound polypeptide complex was detected using a standard horse radish peroxidase conjugate secondary antibody. The concentration of polypeptide complex required to achieve 50% maximal signal (EC₀) was calculated in Graphpad Prism.

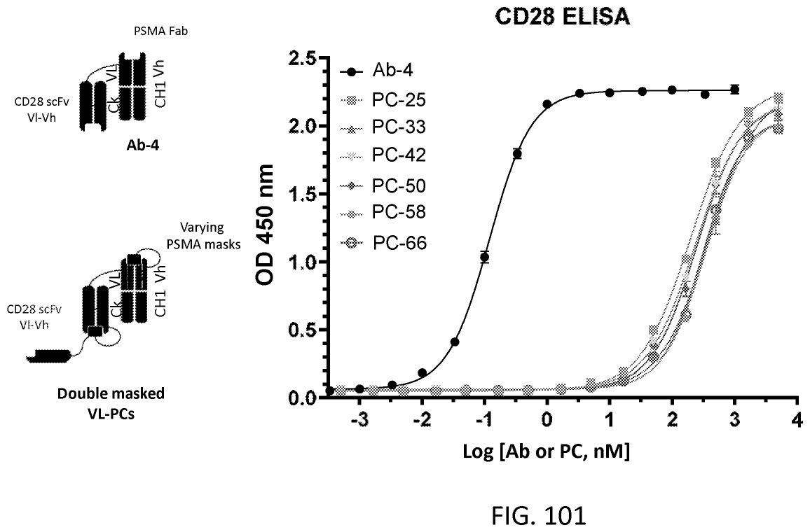

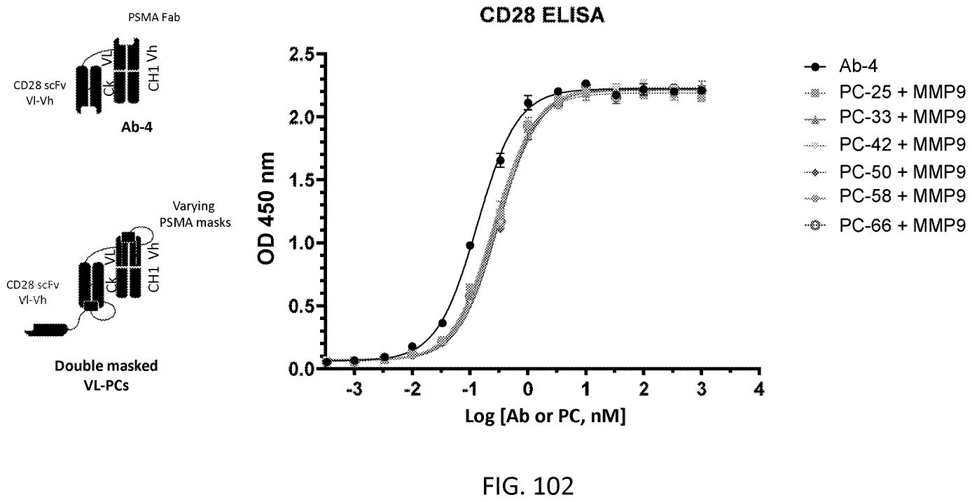

Binding of the polypeptide complexes to CD28 is shown in FIG. 101 Binding of polypeptide complexes to CD28 following treatment with matrix metalloproteinase 9 (MMP9) is shown in FIG. 102. Table 45 summarizes EC₅,) values for the binding of polypeptide complexes to CD28 and to CD28 following MMP9 treatment. The ratio of the EC₅₀ shift is used to calculate the CD28 masking efficiency. Nominal differences were observed in CD28 masking efficiency across the polypeptide complexes with the CD28 mask held constant.

TABLE 45

| | | | CD28 | Cleaved PC, | |
|---|---|---|---|---|---|
| Polypeptide | PSMA | CD28 | EC₅₀ | CD28 | EC₅₀ |
| Complex | Mask | Mask | (nM) | EC₅₀ | shift |
| Ab-4 | — | — | 0.1 | 0.1 | — |
| PC-25 | Peptide-219 | Peptide-41 | 197.2 | 0.2 | 1660x |
| PC-33 | Peptide-230 | Peptide-41 | 378.0 | 0.3 | 3182x |
| PC-42 | Peptide-233 | Peptide-41 | 236.8 | 0.3 | 1993x |
| PC-50 | Peptide-268 | Peptide-41 | 249.0 | 0.3 | 2096x |
| PC-58 | Peptide-271 | Peptide-41 | 342.1 | 0.3 | 2880x |
| PC-66 | Peptide-274 | Peptide-41 | 311.3 | 0.3 | 2620x |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alteratives to the embodiments of the disclosure described herein may be employed in practicing the disclosure. It is intended that the following claims define the scope of the disclosure and that methods and structures within the scope of these claims and their equivalents be covered thereby.

What is claimed is:

1. An isolated polypeptide or polypeptide complex that comprises a first antigen recognizing molecule that binds to CD28 and a second antigen recognizing molecule that binds to prostate-specific membrane antigen (PSMA).

2. The isolated polypeptide or polypeptide complex of claim 1, wherein the first antigen recognizing molecule that binds to CD28 comprises an anti-CD28 antibody.

3. The isolated polypeptide or polypeptide complex of claim 2, wherein the anti-CD28 antibody comprises an anti-CD28 heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 1, HC-CDR2: SEQ ID NO: 2, and HC-CDR3: SEQ ID NO: 3 and the anti-CD28 antibody comprises an anti-CD28 light chain variable domain that comprises complementarity determining regions (CDRs): LC-CDR1, LC-CDR2, and LC-CDR3, and wherein the LC-CDR1, the LC-CDR2, and the LC-CDR3 comprise amino acid sequences of LC-CDR1: SEQ ID NO: 4, LC-CDR2: (KAS), and LC-CDR3: SEQ ID NO: 6.

4. The isolated polypeptide or polypeptide complex of claim 3, wherein the anti-CD28 heavy chain variable domain comprises an amino acid sequence with at least 80%, 85%, 90%, 95%, 98%, 99%, or 100% sequence identity to SEQ ID NO: 7, and wherein the anti-CD28 light chain variable domain comprises an amino acid sequence with at least 80%, 85%, 90%, 95%, 98%, 99%, or 100% sequence identity to SEQ ID NO: 8.

5. The isolated polypeptide or polypeptide complex of claim 3, wherein the anti-CD28 antibody comprises a single chain variable fragment (scFv), a single domain antibody, a Fab, a Fab′, or a CrossFab.

6. The isolated polypeptide or polypeptide complex of claim 5, wherein the anti-CD28 antibody comprises the scFv, and the scFv comprises an amino acid sequence with at least 80%, 85%, 90%, 95%, 98%, 99%, or 100% sequence identity to SEQ ID NO: 9.

7. The isolated polypeptide or polypeptide complex of claim 5, wherein the anti-CD28 antibody comprises the scFv, and the scFv comprises an amino acid sequence with at least 80%, 85%, 90%, 95%, 98%, 99/, or 100% sequence identity to SEQ ID NO: 10.

8. The isolated polypeptide or polypeptide complex of claim 5, wherein the anti-CD28 antibody comprises the scFv, and the scFv comprises an amino acid sequence with at least 80%, 85%, 90%, 95%, 98%, 99%, or 100% sequence identity to SEQ ID NO: 11.

9. The isolated polypeptide or polypeptide complex of claim 5, wherein the anti-CD28 antibody comprises the scFv, and the scFv comprises an amino acid sequence with at least 80%, 85%, 90%, 95%, 98%, 99%, or 100% sequence identity to SEQ ID NO: 12.

10. The isolated polypeptide or polypeptide complex of claim 5, wherein the anti-CD28 antibody comprises the Fab.

11. The isolated polypeptide or polypeptide complex of claim 10, wherein the Fab comprises an amino acid sequence with at least 80%, 85%, 90%, 95%, 98%, 99%, or 100% sequence identity to SEQ ID NO: 696 and an amino acid sequence with at least 80%, 85%, 90%, 95%, 98%, 99%, or 100% sequence identity to SEQ ID NO: 697.

12. The isolated polypeptide or polypeptide complex of claim 5, wherein the anti-CD28 antibody comprises the CrossFab.

13. The isolated polypeptide or polypeptide complex of claim 12, wherein the CrossFab comprises an amino acid sequence with at least 80%, 85%, 90%, 95%, 98%, 99%, or 100% sequence identity to SEQ ID NO: 698 and an amino acid sequence with at least 80%, 85%, 90%, 95%, 98/, 99%, or 100% sequence identity to SEQ ID NO: 699.

14. The isolated polypeptide or polypeptide complex of claim 12, wherein the CrossFab comprises an amino acid sequence with at least 80%, 85%, 90%, 95%, 98%, 99%, or 100% sequence identity to SEQ ID NO: 700 and an amino acid sequence with at least 80%, 85%, 90%, 95%, 98%, 99%, or 100% sequence identity to SEQ ID NO: 701.

15. The isolated polypeptide or polypeptide complex of claim 3, wherein the second antigen recognizing molecule that binds to PSMA comprises an anti-PSMA antibody.

16. The isolated polypeptide or polypeptide complex of claim 15, wherein the anti-PSMA antibody comprises an anti-PSMA heavy chain variable domain that comprises complementarity determining regions (CDRs): HC-CDR1, HC-CDR2, and HC-CDR3, wherein the HC-CDR1, the HC-CDR2, and the HC-CDR3 comprise amino acid sequences of HC-CDR1: SEQ ID NO: 13, HC-CDR2: SEQ ID NO: 14, and HC-CDR3: SEQ ID NO: 15 and the anti-PSMA antibody comprises an anti-PSMA light chain variable domain that comprises complementarity determining regions (CDRs): LC-CDR1, LC-CDR2, and LC-CDR3, wherein the LC-CDR1, the LC-CDR2, and the LC-CDR3 of the light chain variable domain comprise an amino acid sequence of LC-CDR1: SEQ ID NO: 16, LC-CDR2: (EA), and LC-CDR3: SEQ ID NO: 18.

17. The isolated polypeptide or polypeptide complex of claim 16, wherein the anti-PSMA heavy chain variable domain comprises an amino acid sequence with at least 80%, 85%, 90%, 95%, 98%, 99%, or 100% sequence identity to SEQ ID NO: 20, and wherein the anti-PSMA light chain variable domain comprises an amino acid sequence with at least 80%, 85%, 90%, 95%, 98%, 99%, or 100% sequence identity to SEQ ID NO: 19.

18. The isolated polypeptide or polypeptide complex of claim 16, wherein the anti-PSMA antibody comprises a single chain variable fragment (scFv), a single domain antibody, a Fab, a Fab′, or a CrossFab.

19. The isolated polypeptide or polypeptide complex of claim 18, wherein the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 19 and an amino acid sequence with at least at least 80%, 85%, 90%, 95%, 98%, 99%, or 1000/sequence identity to SEQ ID NO: 20.

20. The isolated polypeptide or polypeptide complex of claim 18, wherein the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 702 and an amino acid sequence with at least at least 80%, 85%, 90%, 95%, 98%, 99%, or 100% sequence identity to SEQ ID NO: 703.

21. The isolated polypeptide or polypeptide complex of claim 18, wherein the anti-PSMA antibody comprises the Fab or Fab′, and the Fab or Fab′ comprises an amino acid sequence with at least 80%, 85%, 90%, 95%, 98%, 99%, or 100% sequence identity to SEQ ID NO: 704 and an amino acid sequence with at least at least 80%, 85%, 90%, 95%, 98%, 99%, or 100% sequence identity to SEQ ID NO: 705.

22. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 23 and SEQ ID NO: 24.

23. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 25 and SEQ ID NO: 26.

24. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 27 and SEQ ID NO: 28.

25. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 29 and SEQ ID NO: 30.

26. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 31 and SEQ ID NO: 32.

27. The isolated polypeptide or polypeptide complex of claim 1, wherein the isolated polypeptide or polypeptide complex comprises the amino acid sequences of SEQ ID NO: 25 and SEQ ID NO: 26.

28. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 706, and SEQ ID NO: 707.

29. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 708, SEQ ID NO: 709, and SEQ ID NO: 710.

30. The isolated polypeptide or polypeptide complex of claim 1, wherein the isolated polypeptide or polypeptide complex comprises amino acid sequences with at least 80%, 85%, 90%, 95%, 99%, or 100% sequence identity to SEQ ID NO: 711, SEQ ID NO: 712, and SEQ ID NO: 713.

31. The isolated polypeptide or polypeptide complex of claim 1, wherein the isolated polypeptide or polypeptide complex comprises amino acid sequences with at least 80%, 85%, 90%, 95%, 98%, 99%, or 1000/sequence identity to SEQ ID NO: 714, SEQ ID NO: 715, and SEQ ID NO: 716.

32. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 717, SEQ ID NO: 718, and SEQ ID NO: 719.

33. The isolated polypeptide or polypeptide complex of claim 1, wherein the isolated polypeptide or polypeptide complex comprises amino acid sequences with at least 80%, 85%, 90%, 95%, 98%, 99% or 100% sequence identity to SEQ ID NO: 720, SEQ ID NO: 721, and SEQ ID NO: 722.

34. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 723, SEQ ID NO: 724, and SEQ ID NO: 725.

35. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 726, SEQ ID NO: 727, and SEQ ID NO: 728.

36. The isolated polypeptide or polypeptide complex of claim 1, wherein the isolated polypeptide or polypeptide complex comprises amino acid sequences with at least 80%, 85%, 90%, 95%, 98%, 99%, or 100% sequence identity to SEQ ID NO: 729, SEQ ID NO: 730, and SEQ ID NO: 731.

37. The isolated polypeptide or polypeptide complex of claim 16, wherein the isolated polypeptide or polypeptide complex comprises a peptide that is linked to the anti-CD28 antibody, wherein the peptide impairs binding of the anti-CD28 antibody to CD28.

38. The isolated polypeptide or polypeptide complex of claim 37, wherein the isolated polypeptide or polypeptide complex comprises a configuration according to Formula I: A₂-A₁-L₁-P₁-H₁, wherein A₂ comprises the anti-PSMA antibody, A₁ comprises the anti-CD28 antibody, L₁ comprises a linking moiety that connects A₁ to P₁ and is a substrate for a tumor specific protease, P₁ comprises the peptide that impairs binding of the anti-CD28 antibody to CD28, and H₁ comprises a half-life extending molecule.

39. The isolated polypeptide or polypeptide complex of claim 38, wherein A₂ further comprises P₂ and L₂, wherein P₂ comprises a peptide that binds to A₂; and L₂ comprises a linking moiety that connects A₂ to P₂ and is a substrate for a tumor specific protease.

40. The isolated polypeptide or polypeptide complex of claim 39, wherein the polypeptide or polypeptide complex is according to Formula Ia: P₂-L₂-A₂-A₁-L₁-P₁-H₁.

41. The isolated polypeptide or polypeptide complex of claim 40, wherein P₁ comprises an amino acid sequence according to any one of SEQ ID NOs: 33 to 106, or 732-813.

42. The isolated polypeptide or polypeptide complex of claim 40, wherein P₁ comprises the amino acid sequence of SEQ ID NO: 73.

43. The isolated polypeptide or polypeptide complex of claim 40, wherein P₁ comprises the amino acid sequence of SEQ ID NO: 103.

44. The isolated polypeptide or polypeptide complex of claim 40, wherein P₁ comprises an amino acid sequence according to X₁-X₂-X₃-C-X₄-X₅-X₆-X₇-X₈-X₉-X₁₀-C-X₁₁-X₁: wherein X₁ is selected from M, I, L, and V; X₂ is selected from D, H, N, A, F, S, T, Y, and V; X₃ is selected from W, L, and F; X₄ is selected from P, A, and L: X₅ is selected from R, T, I, M, S, K, L, V, W, F, A, P, and D; X₆ is selected from E, D, Y, H, S, F, A, N, T, I, P, and V; X₇ is selected from L, M, R, S, Q, and H; X₈ is selected from W and Q: X₉ is selected from H, N, D, A, S, Y, T, F, V, L, and I; X₁₀ is selected from E, V, L, D, Y, R, Q, H, F, K, A, M, and N; X₁₁ is selected from F, Y, L, W, and V; and X₁₂ is selected from N, A, F, S, Y, H, D, T, and L.

45. The isolated polypeptide or polypeptide complex of claim 44, wherein X₁ is selected from M, I, and L: X₂ is selected from D, H, N, and A: X₃ is W: X₄ is P; X₅ is selected from R, T, I, M, S, and K; X₆ is selected from E, D, Y, H, S, and F; X₇ is selected from L, M, and R; X₈ is W; X₉ is selected from H, N, D, A, S, and V; X₁₀ is selected from E, V, L, D, and H; X₁₁ is selected from F, Y, and L; and X₁₂ is selected from N, A, F, S, and Y.

46. The isolated polypeptide or polypeptide complex of claim 40, wherein P₁ comprises an amino acid sequence according to SEQ ID NO: 41.

47. The isolated polypeptide or polypeptide complex of claim 40, wherein P₁ comprises an amino acid sequence according to: J₁-J₂-W-C-J₃-J₄-J₅-J₆-J₇-J₈-J₉-C-J₁₀-J₁₁, wherein:
J₁ is selected from A, R, N, D, Q, E, G, H, I, L, K, F, P, S, T, W, Y, and V;
J₂ is selected from D, P, and Y;
J₃ is selected from P and Q;
J₄ is selected from R, L, and I;
J₅ is selected from D, H, Y, and I;
J₆ is selected from L, S, and R;
J₇ is selected from W and G;
J₈ is selected from V, A, D, H, L, and N;
J₉ is selected from H, L, E, and D:
J₁₀ is selected from F, Y, and L; and
J₁₁ is selected from A, N, H, and T.

48. The isolated polypeptide or polypeptide complex of claim 47, wherein:
J₁ is selected from A, R, N, D, Q, E, G, H, K, F, P, S, T, W, and Y.

49. The isolated polypeptide or polypeptide complex of claim 47, wherein:
J₁ is selected from R, Q, E, H, I, L, K, F, T, W, Y, and V;
J₂ is D;
J₃ is P;
J₄ is R;
J₅ is D;
J₆ is L:
J₇ is W;
J₈ is V;
J₉ is H;
J₁₀ is F; and
J₁₁ is A.

50. The isolated polypeptide or polypeptide complex of claim 40, wherein P₁ comprises an amino acid sequence according to: Z₁-C-Z₂-Z₃-Z₄-Z₅-Z₆-Z₇-Z₈-Z₉-Z₁₀-Z₁₁-C-Z₁₂, wherein:
Z₁ is selected from Y, H, and A;
Z₂ is selected from D, E, A, Q, S, V, M, H, L, F, T, W, and N:
Z₃ is selected from G, E, S, and A:
Z₄ is selected from H, L, W, and A;
Z₅ is selected from L, S, A, T, I, V, M, R, E, D, F, H K, Q, N, Y, W, and G;
Z₆ is selected from P, A, E, Q, S, L, W, G, V, D, K, I, M, R, T, F, H, N, and Y:
Z₇ is selected from E, Q, I, M, V, A, L, R, W, P, D, T, K, S, G, H, and N:
Z₈ is selected from Y, H, F, W, N, Q, and A;
Z₉ is selected from M, I, L, R, V, K, F, Q, Y, W, and A;
Z₁₀ is selected from A, Q, S, W, E, L, G, D, T, M, N, V, H, R, Y, K, P, F, and I;
Z₁₁ is selected from K, Q, N, H, E, P, and A; and
Z₁₂ is selected from L, M, I, F, V, Y, Q, D, T, and A.

51. The isolated polypeptide or polypeptide complex of claim 50, wherein:
Z₁ is selected from Y and H;
Z₂ is selected from D, E, A, Q, S, V, M, H, L, F, T, W, and N;
Z₃ is selected from G, E, and S;
Z₄ is selected from H, L, and W;
Z₅ is selected from L, S, A, T, I, V, M, R, E, D, F, H, K, Q, N, Y, W, and G;
Z₆ is selected from P, A, E, Q, S, L, W, G, V, D, K, I, M, R, T, F, H, N, and Y;
Z₇ is selected from E, Q, I, M, V, A, L, R, W, P, D, T, K, S, G, H, and N;
Z₈ is selected from Y, H, F, W, N, and Q;
Z₉ is selected from M, I, L, R, V, K, F, Q, Y, and W:
Z₁₀ is selected from A, Q, S, W, E, L, G, D, T, M, N, V, H, R, Y, K, P, F, and I;
Z₁₁ is selected from K, Q, N, H, E, and P; and
Z₁₂ is selected from L, M, I, F, V, Y, Q, D, and T.

52. The isolated polypeptide or polypeptide complex of claim 40, wherein P₁ comprises the amino acid sequence of SEQ ID NO: 784.

53. The isolated polypeptide or polypeptide complex of claim 40, wherein P₁ comprises the amino acid sequence of SEQ ID NO: 744.

54. The isolated polypeptide or polypeptide complex of claim 40, wherein P, consists of the amino acid sequence of SEQ ID NO: 744.

55. The isolated polypeptide or polypeptide complex of claim 40, wherein P₁ comprises an amino acid sequence according to any one of SEQ ID NOs: 774-793.

56. The isolated polypeptide or polypeptide complex of claim 40, wherein the peptide comprises an amino acid sequence according to any one of SEQ ID NOs: 764-773.

57. The isolated polypeptide or polypeptide complex of claim 40, wherein P₁ comprises an amino acid sequence according to: U₁-C-U₂-U₃-U₄-U₅-U₆-U₇-U₈-U₉-U₁₀-U₁₁-C-U₁₂, wherein:
U₁ is selected from T, S, L, M, W, Q, P, R, G, N, I, Y, D, E, V, H, and A;
U₂ is selected from A, S, T, K, E, R, G, N, W, and D;
U₃ is selected from A, E, P, D, G, W, Q, V, and F;
U₄ is selected from G, V, F, D, E, R, and A;
U₅ is selected from A, F, V, S, L, P, and M;
U₆ is selected from T, S, H, M, A, N, P, G, Q, D, Y, V, L, I, E, K, F, and R;
U₇ is selected from Y, W, F, L, N, T, Q, I, and A;
U₈ is selected from P, S, T, A, E, K, Q, N, V, G, H, L, D, Y, I, M, and R;
U₉ is selected from W, L, and A;
U₁₀ is selected from P, S, R, L, E, and A;
U₁₁ is selected from L, A, T, M, V, W, Y, E, Q, I, F, S, P, K, R, D, H, N, and G; and
U₁₂ is selected from L, F, V, M, I, Y, E, W, and A.

58. The isolated polypeptide or polypeptide complex of claim 57, wherein:
U₁ is selected from T, S, L, M, W, Q, P, R, G, N, I, Y, D, E, V, and H;
U₂ is selected from A, S, T, K, E, R, G, N, W, and D;
U₃ is selected from A, E, P, D, G, W, Q, V, and F;
U₄ is selected from G, V, F, D, E, and R;
U₅ is selected from A, F, V, S, L, P, and M:
U₆ is selected from T, S, H, M, A, N, P, G, Q, D, Y, V, L, I, E, K, F, and R;
U₇ is selected from Y, W, F, L, N, T, Q, and 1;
U₈ is selected from P, S, T, A, E, K, Q, N, V, G, H, L, D, Y, I, M, and R;
U₉ is selected from W, L, and A;
U₁₀ is selected from P, S, R, L, and E:
U₁₁ is selected from L, A, T, M, V, W, Y, E, Q, I, F, S, P, K, R, D, H, N, and G; and
U₁₂ is selected from L, F, V, M, I, Y, E, and W.

59. The isolated polypeptide or polypeptide complex of claim 40, wherein P₁ comprises the amino acid sequence of SEQ ID NO: 749.

60. The isolated polypeptide or polypeptide complex of claim 40, wherein P₁ comprises an amino acid sequence according to any one of SEQ ID NOs: 756-763.

61. The isolated polypeptide or polypeptide complex of claim 40, wherein P₁ comprises an amino acid sequence according to any one of SEQ ID NOs: 794-813.

62. The isolated polypeptide or polypeptide complex of claim 40, wherein P₁ comprises an amino acid sequence according to any one of SEQ ID NOs: 732-2002.

63. The isolated polypeptide or polypeptide complex of claim 40, wherein P₂ comprises the amino acid sequence of any one of SEQ ID NOs: 20034329.

64. The isolated polypeptide or polypeptide complex of claim 40, wherein P₂ comprises the amino acid sequence of SEQ ID NO: 2025.

65. The isolated polypeptide or polypeptide complex of claim 40, wherein P₂ comprises the amino acid sequence according to any one of SEQ ID NOs: 2087-2094.

66. The isolated polypeptide or polypeptide complex of claim 40, wherein P₂ comprises the amino acid sequence of SEQ ID NO: 2026.

67. The isolated polypeptide or polypeptide complex of claim 40, wherein P₂ comprises the amino acid sequence according to any one of SEQ ID NOs: 2097-2106.

68. The isolated polypeptide or polypeptide complex of claim 40, wherein P₂ comprises the amino acid sequence according to any one of SEQ ID NOs: 2107-2133.

69. The isolated polypeptide or polypeptide complex of claim 40, wherein P₂ comprises the amino acid sequence of SEQ ID NO: 2120.

70. The isolated polypeptide or polypeptide complex of claim 40, wherein P₂ comprises an amino acid sequence according to B₁-B₂-B₃-B₄-C-B₅-P-B₆-W-B₇-C-B₈-B₉-B₁₀ (SEQ ID NO: 4470), wherein:
B₁ is selected from V, E, L, D, I, G, M, S, P, T, A, F, W, Y, Q, H, N, K, and R;
B₂ is selected from E, V, D, T, S, L, G, P, A, M, I, Q, H, F, Y, N, W, R, and K;
B₃ is selected from K, P, R, I, N, H, V, M, A, L, Q, T, S, G, F, Y, E, W, and D;
B₄ is selected from W, L, M, R, V, Y, A, K, I, S, Q, F, H, E, T, N, G, and D;
B₅ is selected from I, V, T, K, R, E, S, Q, M, L, F, A, N, and H;
B₆ is selected from A, E, S, P, Q, T, L, D, M, V, R, K, N, I, H, W, Y, F, and G;
B₇ is selected from M, L, I, A, V, F, G, and K:
B₈ is selected from E, S, T, A, V, D, Q, L, N, I, M, Y, H, F, W, G, K, R, and P;
B₉ is selected from G, V, A, S, W, E, D, M, T, L, F, H, Q, N, R, I, Y,P, and K; and
B₁₀ is selected from F, L, M, S, I, V, D, Q, T, A, N, Y, W, E, R, H, P, G, and K.

71. The isolated polypeptide or polypeptide complex of claim 40, wherein P₁ becomes unbound from A, when L₁ is cleaved by the tumor specific protease thereby exposing A₁ to CD28.

72. The isolated polypeptide or polypeptide complex of claim 40, wherein P₂ becomes unbound from A₂ when L₂ is cleaved by the tumor specific protease thereby exposing A₂ to PSMA.

73. The isolated polypeptide or polypeptide complex of claim 40, wherein L₁ or L₂ comprises a urokinase cleavable amino acid sequence, a matriptase cleavable amino acid sequence, matrix metalloprotease cleavable amino acid sequence, or a legumain cleavable amino acid sequence.

74. The isolated polypeptide or polypeptide complex of claim 40, wherein L₁ or L₂ comprises an amino acid sequence according to any one of SEQ ID NOs: 107-137 and 604.

75. The isolated polypeptide or polypeptide complex of claim 40, wherein the half-life extending molecule comprises a single domain antibody.

76. The isolated polypeptide or polypeptide complex of claim 75, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 138, HC-CDR2: SEQ ID NO: 139, and HC-CDR3: SEQ ID NO: 140.

77. The isolated polypeptide or polypeptide complex of claim 75, wherein the single domain antibody comprises complementarity determining regions (CDRs): HC-CDR1, HC-CDR2, and HC-CDR3, wherein the HC-CDR1, the HC-CDR2, and the HC-CDR3 of the single domain antibody comprise: HC-CDR1: SEQ ID NO: 142, HC-CDR2: SEQ ID NO: 143, and HC-CDR3: SEQ ID NO: 144.

78. The isolated polypeptide or polypeptide complex of claim 75, wherein the single domain antibody comprises an amino acid sequence with at least 80%, 85%, 900, 95%, 99%, or 100% identity to SEQ ID NO: 141.

79. The isolated polypeptide or polypeptide complex of claim 75, wherein the single domain antibody comprises an amino acid sequence with at least 80%, 85%, 90%, 95%, 99%, or 100% identity to SEQ ID NO: 146.

80. The isolated polypeptide or polypeptide complex of claim 75, wherein the single domain antibody comprises an amino acid sequence with at least 80%, 85%, 90%, 95%, 99%, or 100% identity to SEQ ID NO: 145.

81. The isolated polypeptide or polypeptide complex of claim 75, wherein the single domain antibody comprises an amino acid sequence with at least 80%, 85%, 90%, 95%, 99%, or 100% identity to SEQ ID NO: 4448.

82. The isolated polypeptide or polypeptide complex of claim 1, wherein the polypeptide or polypeptide complex comprises amino acid sequences with at least 95% sequence identity to SEQ ID NOs: 147 and 148.

83. The isolated polypeptide or polypeptide complex of claim 1, wherein the polypeptide or polypeptide complex comprises amino acid sequences with at least 95% sequence identity to SEQ ID NOs: 149 and 150.

84. The isolated polypeptide or polypeptide complex of claim 1, wherein the polypeptide or polypeptide complex comprises amino acid sequences with at least 95% sequence identity to SEQ ID NOs: 151 and 152.

85. The isolated polypeptide or polypeptide complex of claim 1, wherein the polypeptide or polypeptide complex comprises amino acid sequences with at least 95% sequence identity to SEQ ID NOs: 153 and 154.

86. The isolated polypeptide or polypeptide complex of claim 1, wherein the polypeptide or polypeptide complex comprises amino acid sequences with at least 95% sequence identity to SEQ ID NOs: 155 and 156.

87. The isolated polypeptide or polypeptide complex of claim 1, wherein the polypeptide or polypeptide complex comprises amino acid sequences with at least 95% sequence identity to SEQ ID NOs: 157 and 158.

88. The isolated polypeptide or polypeptide complex of claim 1, wherein the polypeptide or polypeptide complex comprises amino acid sequences with at least 95% sequence identity to SEQ ID NOs: 159 and 160.

89. The isolated polypeptide or polypeptide complex of claim 1, wherein the polypeptide or polypeptide complex comprises amino acid sequences with at least 95% sequence identity to SEQ ID NOs: 161 and 162.

90. The isolated polypeptide or polypeptide complex of claim 1, wherein the polypeptide or polypeptide complex comprises amino acid sequences with at least 95% sequence identity to SEQ ID NOs: 163 and 164.

91. The isolated polypeptide or polypeptide complex of claim 1, wherein the polypeptide or polypeptide complex comprises amino acid sequences with at least 95% sequence identity to SEQ ID NOs: 165 and 166.

92. The isolated polypeptide or polypeptide complex of claim 1, wherein the polypeptide or polypeptide complex comprises amino acid sequences with at least 95% sequence identity to SEQ ID NOs: 167 and 168.

93. The isolated polypeptide or polypeptide complex of claim 1, wherein the polypeptide or polypeptide complex comprises amino acid sequences with at least 95% sequence identity to SEQ ID NOs: 169 and 170.

94. The isolated polypeptide or polypeptide complex of claim 1, wherein the polypeptide or polypeptide complex comprises amino acid sequences with at least 95% sequence identity to SEQ ID NOs: 171 and 172.

95. The isolated polypeptide or polypeptide complex of claim 1, wherein the polypeptide or polypeptide complex comprises amino acid sequences with at least 95% sequence identity to SEQ ID NOs: 173 and 174.

96. The isolated polypeptide or polypeptide complex of claim 1, wherein the polypeptide or polypeptide complex comprises amino acid sequences with at least 95% sequence identity to SEQ ID NOs: 175 and 176.

97. The isolated polypeptide or polypeptide complex of claim 1, wherein the polypeptide or polypeptide complex comprises amino acid sequences with at least 95% sequence identity to SEQ ID NOs: 177 and 178.

98. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4330 and 4331.

99. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4332 and 4333.

100. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4334 and 4335.

101. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4336 and 4337.

102. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4338 and 4339.

103. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4340 and 4341.

104. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4342 and 4343.

105. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4344 and 4345.

106. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4346 and 4347.

107. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4348 and 4349.

108. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4350 and 4351.

109. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4352 and 4353.

110. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4354 and 4355.

111. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4356 and 4357.

112. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4358 and 4359.

113. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4360 and 4361.

114. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4362 and 4363.

115. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4364 and 4365.

116. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4366 and 4367.

117. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4368 and 4369.

118. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4370 and 4371.

119. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4372 and 4373.

120. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4374 and 4375.

121. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4376 and 4377.

122. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4378 and 4379.

123. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4380 and 4381.

124. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4382 and 4383.

125. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4384 and 4385.

126. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4386 and 4387.

127. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4388 and 4389.

128. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4390 and 4391.

129. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4392 and 4393.

130. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4394 and 4395.

131. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4396 and 4397.

132. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4398 and 4399.

133. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4400 and 4401.

134. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4402 and 4403.

135. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4404 and 4405.

136. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4406 and 4407.

137. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4408 and 4409.

138. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4410 and 4411.

139. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4412 and 4413.

140. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4414 and 4415.

141. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4416 and 4417.

142. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4418 and 4419.

143. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4420 and 4421.

144. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4422 and 4423.

145. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4424 and 4425.

146. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4426 and 4427.

147. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4428 and 4429.

148. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4430 and 4431.

149. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4432 and 4433.

150. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4434 and 4435.

151. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4436 and 4437.

152. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4438 and 4439.

153. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4440 and 4441.

154. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4442 and 4443.

155. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4444 and 4445.

156. The isolated polypeptide or polypeptide complex of claim 1, wherein the polypeptide or polypeptide complex comprises amino acid sequences with at least 80%, 85%, 90%, 95%, 99%, or 100% sequence identity to SEQ ID NO: 4446 and 4447.

157. An isolated recombinant nucleic acid molecule encoding the polypeptide or polypeptide complex of claim 1.

158. A pharmaceutical composition comprising:
(a) the polypeptide or polypeptide complex of claim 1; and
(b) a pharmaceutically acceptable excipient.

159. A method of treating cancer in a subject in need thereof comprising administering to the subject the polypeptide or polypeptide complex of claim 1.

160. The method of claim 159, wherein the polypeptide or polypeptide complex is administered in combination with an anti-cancer therapy.

161. The method of claim 160, wherein the anti-cancer therapy comprises a small molecule, a cell-based therapy, or an antibody-based therapy.

162. The method of claim 161, wherein the antibody-based therapy is a T cell engager.

163. A vector comprising the isolated recombinant nucleic acid molecule of claim 157.
